(19) United States
(12) Patent Application Publication
(10) Pub. No.: US 2020/0188496 A1
(43) Pub. Date: Jun. 18, 2020

(54) UNIVERSAL CANCER VACCINES AND METHODS OF MAKING AND USING SAME

(71) Applicant: ARIZONA BOARD OF REGENTS ON BEHALF OF ARIZONA STATE UNIVERSITY, Bcottsdale, AZ (US)

(72) Inventors: Stephen Albert JOHNSTON, Tempe, AZ (US); Luhui SHEN, Tempe, AZ (US)

(21) Appl. No.: 16/617,830

(22) PCT Filed: Jun. 1, 2018
(86) PCT No.: PCT/US2018/035743

§ 371 Date: Nov. 27, 2019

Related U.S. Application Data
(60) Provisional application No. 62/514,674, filed on Jun. 2, 2017.

(51) Int. Cl. A61K 39/00; C07K 16/30; A61K 39/39; A61P 35/00

(52) U.S. Cl. CPC A61K 39/0011; C07K 16/30; A61K 39/39; A61P 35/00; C07K 16/2818; A61K 2039/54; A61K 2039/70; A61K 2039/812; A61K 2039/505

(57) ABSTRACT

Disclosed herein are compositions comprising a universal cancer vaccine and methods of treating and preventing cancer using such compositions.

### CROSS-REFERENCE

This application claims the benefit of U.S. Provisional Application No. 62/514,674, filed Jun. 2, 2017, which is incorporated herein by reference in its entirety.

### BACKGROUND

Treating cancer poses multiple challenges. By the time the cancer is detected it has often metastasized, requiring systemic treatment, such as chemotherapy. In most cases, once detected, the cancer has become heterogeneous, allowing a subpopulation of cells to become resistant to certain treatments. In addition, the cancer can evolve resistance in response to the treatment. In spite of encouraging advances in immunotherapeutics, there is still a need for novel cancer therapies.

Cancer immunotherapy uses a patient's immune system to treat cancer. Types of immunotherapy include monoclonal antibodies, immune checkpoint inhibitors, adjuvants, CAR-T cell therapy, and cancer vaccines. Cancer vaccines most commonly available at this time involve vaccinating against viruses known to cause cancer, such as human papillomaviruses and hepatitis B. However, no vaccines for preventing cancer are available for cancers not caused by an infectious agent. For therapeutic vaccination to treat cancer it is generally assumed that such a vaccine would necessarily be unique to each individual being treated. This is based on the realizations that common self-antigens have failed as vaccines, neo-epitopes produced by the tumor are the best antigens and that almost all the DNA encoded neo-antigens developed to date are unique to each individual.

### SUMMARY

We have developed a method to compose vaccines that are broadly protective or therapeutic for cancer. Provided herein are methods of treating a cancer in an individual in need of treatment. Some such methods comprise: administering one or more peptide(s), wherein at least one peptide comprises a variant in a microsatellite (MS) coding region of an expressed gene, wherein the one or more peptide(s) are at least 8 amino acids in length and wherein administration of the peptide(s) or nucleic acid(s) encoding the one or more peptide(s) results in an immune response to the cancer. In some cases, the methods comprise administering one or more peptides selected from the group consisting of peptides in SEQ ID NO: 1-6554 or a nucleic acid encoding the peptide(s), wherein administration of the peptide(s) or the nucleic acid(s) results in an immune response to the cancer. In some cases, methods comprise administering two or more peptides selected from the group consisting of peptides in SEQ ID NO: 1-6554. In some cases, methods comprise administering three or more peptides selected from the group consisting of peptides in SEQ ID NO: 1-6554. In some cases, methods comprise administering four or more peptides selected from the group consisting of peptides in SEQ ID NO: 1-6554. In some cases, methods comprise administering five or more peptides selected from the group consisting of peptides in SEQ ID NO: 1-6554. In some cases, methods comprise administering six or more peptides selected from the group consisting of peptides in SEQ ID NO: 1-6554. In some cases, methods comprise administering seven or more peptides selected from the group consisting of peptides in SEQ ID NO: 1-6554. In some cases, methods comprise administering eight or more peptides selected from the group consisting of peptides in SEQ ID NO: 1-6554. In some cases, methods comprise administering nine or more peptides selected from the group consisting of peptides in SEQ ID NO: 1-6554. In some cases, methods comprise administering ten or more peptides selected from the group consisting of peptides in SEQ ID NO: 1-6554. In some cases, methods comprise administering 11, 12, 13, 14, 15, 16, 17, 18, 19, 20 or more peptides selected from the group consisting of peptides in SEQ ID NO: 1-6554. Often, the immune response to the cancer is an antibody response. Alternatively, or in combination, the immune response to the cancer is a T cell response. In some cases, treating the cancer comprises reducing tumor size, inhibiting tumor growth, reducing tumor burden, decreasing metastatic cancer burden, increasing survival, or increasing cancer-free survival. Often, the peptide(s) are encoded by an mRNA comprising a frameshift variant expressed by a cancer cell. In some cases, the frameshift variant is created by a transcription insertion or deletion error in a microsatellite. In some cases, the frameshift variant is downstream of a microsatellite. Often, the peptides comprise at least one T cell epitope. Alternatively or in combination, the peptides comprise at least one B cell epitope. In some cases, the peptides are administered as a nucleic acid encoding the peptides, such as a single nucleic acid encoding all of the peptides. Alternatively, peptides administered as a nucleic acid encoding the peptides are each encoded by a separate nucleic acid molecule. Often, the peptides are administered in a vaccine composition. Some such vaccine compositions often comprise an adjuvant. Such adjuvants are selected from the group consisting of ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan. Often, the method further comprises administration of a checkpoint inhibitor. Some such checkpoint inhibitors are selected from the group consisting of a PD-1 inhibitor, a PD-L1 inhibitor, and a CTLA-4 inhibitor. Alternatively, or in combination, immune checkpoint inhibitors are selected from one or more of the group consisting of Pembrolizumab, Nivolumab, and Atezolizumab. In some cases, peptides are administered via a route selected from the group consisting of subcutaneous, oral, mucosal, intradermal, intramuscular, intranasal, intravenous, and sublingual. In some cases, the individual is a mammal. Often, the individual is a human, a dog, a cat, a mouse, a rat, a rabbit, a horse, a cow, or a pig. Cancers for treatment by methods herein are often selected from the group consisting of Acute lymphoblastic leukemia, Acute monocytic leukemia, Acute myeloid leukemia, Acute promyelocytic leukemia, Adenocarcinoma, Adult T-cell leukemia, Astrocytoma, Bladder cancer, Bone Cancer, Brain Tumor, Breast Cancer, Burkitt's lymphoma, Carcinoma, Cervical Cancer, Chronic Lymphocytic Leukemia, Chronic myelogenous leukemia, Colon Cancer, Colorectal cancer, Endometrial cancer, Glioblastoma multiforme, Glioma, Hepatocellular carcinoma, Hodgkin's lymphoma, Inflammatory breast cancer, Kidney Cancer, Leukemia, Lung cancer, Lymphoma, Malignant Mesothelioma, Medulloblastoma, Melanoma, Multiple myeloma, Neuroblastoma, Non-Hodgkin Lymphoma, Non-Small Cell Lung Cancer, Ovarian Cancer, Pancreatic Cancer, Pituitary tumor, Prostate cancer, Retinoblastoma, Skin Cancer, Small Cell Lung Cancer, Squamous cell carcinoma, Stomach cancer, T-cell leukemia, T-cell lymphoma, Thyroid cancer, and Wilms' tumor.

Also provided herein are methods of preventing cancer in an individual. Some such methods often comprise administering one or more peptides selected from the group consisting of SEQ ID NO: 1-6554 or a nucleic acid encoding the peptide(s), wherein administration of the peptide(s) or the nucleic acid(s) results in an immune response to the cancer. Some such methods comprise administering one or more peptides selected from the group consisting of peptides in SEQ ID NO: 1-6554 or a nucleic acid encoding the peptide(s), wherein administration of the peptide(s) or the nucleic acid(s) results in an immune response to the cancer. In some cases, methods comprise administering two or more peptides selected from the group consisting of peptides in SEQ ID NO: 1-6554. In some cases, methods comprise administering three or more peptides selected from the group consisting of peptides in SEQ ID NO: 1-6554. In some cases, methods comprise administering four or more peptides selected from the group consisting of peptides in SEQ ID NO: 1-6554. In some cases, methods comprise administering five or more peptides selected from the group consisting of peptides in SEQ ID NO: 1-6554. In some cases, methods comprise administering six or more peptides selected from the group consisting of peptides in SEQ ID NO: 1-6554. In some cases, methods comprise administering seven or more peptides selected from the group consisting of peptides in SEQ ID NO: 1-6554. In some cases, methods comprise administering eight or more peptides selected from the group consisting of peptides in SEQ ID NO: 1-6554. In some cases, methods comprise administering nine or more peptides selected from the group consisting of peptides in SEQ ID NO: 1-6554. In some cases, methods comprise administering ten or more peptides selected from the group consisting of peptides in SEQ ID NO: 1-6554. In some cases, methods comprise administering 11, 12, 13, 14, 15, 16, 17, 18, 19, 20 or more peptides selected from the group consisting of peptides in SEQ ID NO: 1-6554. Often, the immune response to the cancer is an antibody response. Alternatively, or in combination, the immune response to the cancer is a T cell response. In some cases, treating the cancer comprises reducing tumor size, inhibiting tumor growth, reducing tumor burden, increasing survival, or increasing cancer-free survival. Often, the peptide(s) are encoded by an mRNA comprising a frameshift variant expressed by a cancer cell. In some cases, the frameshift variant is created by a transcription insertion or deletion error in a microsatellite. In some cases, the frameshift variant is downstream of a microsatellite. Often, the peptides comprise at least one T cell epitope. Alternatively or in combination, the peptides comprise at least one B cell epitope. In some cases, the peptides are administered as a nucleic acid encoding the peptides, such as a single nucleic acid encoding all of the peptides. Alternatively, peptides administered as a nucleic acid encoding the peptides are each encoded by a separate nucleic acid molecule. Often, the peptides are administered in a vaccine composition. Some such vaccine compositions often comprise an adjuvant. Such adjuvants are selected from the group consisting of ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan. Often, the method further comprises administration of a checkpoint inhibitor. Some such checkpoint inhibitors are selected from the group consisting of a PD-1 inhibitor, a PD-L1 inhibitor, and a CTLA-4 inhibitor. Alternatively, or in combination, immune checkpoint inhibitors are selected from one or more of the group consisting of Pembrolizumab, Nivolumab, and Atezolizumab. In some cases, peptides are administered via a route selected from the group consisting of subcutaneous, oral, mucosal, intradermal, intramuscular, intranasal, intravenous, and sublingual. In some cases, the individual is a mammal. Often, the individual is a human, a dog, a cat, a mouse, a rat, a rabbit, a horse, a cow, or a pig. Cancers for treatment by methods herein are often selected from the group consisting of Acute lymphoblastic leukemia, Acute monocytic leukemia, Acute myeloid leukemia, Acute promyelocytic leukemia, Adenocarcinoma, Adult T-cell leukemia, Astrocytoma, Bladder cancer, Bone Cancer, Brain Tumor, Breast Cancer, Burkitt's lymphoma, Carcinoma, Cervical Cancer, Chronic Lymphocytic Leukemia, Chronic myelogenous leukemia, Colon Cancer, Colorectal cancer, Endometrial cancer, Glioblastoma multiforme, Glioma, Hepatocellular carcinoma, Hodgkin's lymphoma, Inflammatory breast cancer, Kidney Cancer, Leukemia, Lung cancer, Lymphoma, Malignant Mesothelioma, Medulloblastoma, Melanoma, Multiple myeloma, Neuroblastoma, Non-Hodgkin Lymphoma, Non-Small Cell Lung Cancer, Ovarian Cancer, Pancreatic Cancer, Pituitary tumor, Prostate cancer, Retinoblastoma, Skin Cancer, Small Cell Lung Cancer, Squamous cell carcinoma, Stomach cancer, T-cell leukemia, T-cell lymphoma, Thyroid cancer, and Wilms' tumor.

Also provided herein are methods of eliciting an immune response in an individual. Some such methods often comprise administering one or more peptides selected from the group consisting of SEQ ID NO: 1-6554 wherein administration of the peptide(s) results in an immune response to the peptides. In some cases, methods comprise administering two or more peptides selected from the group consisting of peptides in SEQ ID NO: 1-6554. In some cases, methods comprise administering three or more peptides selected from the group consisting of peptides in SEQ ID NO: 1-6554. In some cases, methods comprise administering four or more peptides selected from the group consisting of peptides in SEQ ID NO: 1-6554. In some cases, methods comprise administering five or more peptides selected from the group consisting of peptides in SEQ ID NO: 1-6554. In some cases, methods comprise administering six or more peptides selected from the group consisting of peptides in SEQ ID NO: 1-6554. In some cases, methods comprise administering seven or more peptides selected from the group consisting of peptides in SEQ ID NO: 1-6554. In some cases, methods comprise administering eight or more peptides selected from the group consisting of peptides in SEQ ID NO: 1-6554. In some cases, methods comprise administering nine or more peptides selected from the group consisting of peptides in SEQ ID NO: 1-6554. In some cases, methods comprise administering ten or more peptides selected from the group consisting of peptides in SEQ ID NO: 1-6554. In some cases, methods comprise administering 11, 12, 13, 14, 15, 16, 17, 18, 19, 20 or more peptides selected from the group consisting of peptides in SEQ ID NO: 1-6554. Often, the immune response to the cancer is an antibody response. Alternatively, or in combination, the immune response to the cancer is a T cell response. In some cases, preventing the cancer comprises reducing cancer incidence. Often, the peptide(s) are encoded by an mRNA comprising a frameshift variant expressed by a cancer cell. In some cases, the frameshift variant is created by a transcription insertion or deletion error in a microsatellite. In some cases, the frameshift variant is downstream of a microsatellite. Often, the peptides comprise at least one T cell epitope. Alternatively or in combination, the peptides comprise at least one B cell epitope. In some cases, the peptides are administered as a nucleic acid encoding the peptides, such as a single nucleic acid encoding all of the peptides. Alternatively, peptides administered as a nucleic acid encoding the peptides are each encoded by a separate nucleic acid molecule. Often, the peptides are administered in a vaccine composition. Some such vaccine compositions often comprise an adjuvant. Such adjuvants are selected from the group consisting of ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan. Often, the method further comprises administration of a checkpoint inhibitor. Some such checkpoint inhibitors are selected from the group consisting of a PD-1 inhibitor, a PD-L1 inhibitor, and a CTLA-4 inhibitor. Alternatively, or in combination, immune checkpoint inhibitors are selected from one or more of the group consisting of Pembrolizumab, Nivolumab, and Atezolizumab. In some cases, peptides are administered via a route selected from the group consisting of subcutaneous, oral, mucosal, intradermal, intramuscular, intranasal, intravenous, and sublingual. In some cases, the individual is a mammal. Often, the individual is a human, a dog, a cat, a mouse, a rat, a rabbit, a horse, a cow, or a pig. Often the immune response is directed to a cancer. Some such cancers include those selected from the group consisting of Acute lymphoblastic leukemia, Acute monocytic leukemia, Acute myeloid leukemia, Acute promyelocytic leukemia, Adenocarcinoma, Adult T-cell leukemia, Astrocytoma, Bladder cancer, Bone Cancer, Brain Tumor, Breast Cancer, Burkitt's lymphoma, Carcinoma, Cervical Cancer, Chronic Lymphocytic Leukemia, Chronic myelogenous leukemia, Colon Cancer, Colorectal cancer, Endometrial cancer, Glioblastoma multiforme, Glioma, Hepatocellular carcinoma, Hodgkin's lymphoma, Inflammatory breast cancer, Kidney Cancer, Leukemia, Lung cancer, Lymphoma, Malignant Mesothelioma, Medulloblastoma, Melanoma, Multiple myeloma, Neuroblastoma, Non-Hodgkin Lymphoma, Non-Small Cell Lung Cancer, Ovarian Cancer, Pancreatic Cancer, Pituitary tumor, Prostate cancer, Retinoblastoma, Skin Cancer, Small Cell Lung Cancer, Squamous cell carcinoma, Stomach cancer, T-cell leukemia, T-cell lymphoma, Thyroid cancer, and Wilms' tumor.

Also provided herein are cancer vaccines. Some such cancer vaccines often comprise one or more peptides selected from the group consisting of SEQ ID NO: 1-6554 and an adjuvant. In some cases, cancer vaccines comprise two or more peptides selected from the group consisting of peptides in SEQ ID NO: 1-6554. In some cases, cancer vaccines comprise three or more peptides selected from the group consisting of peptides in SEQ ID NO: 1-6554. In some cases, cancer vaccines comprise four or more peptides selected from the group consisting of peptides in SEQ ID NO: 1-6554. In some cases, cancer vaccines comprise five or more peptides selected from the group consisting of peptides in SEQ ID NO: 1-6554. In some cases, cancer vaccines comprise six or more peptides selected from the group consisting of peptides in SEQ ID NO: 1-6554. In some cases, cancer vaccines comprise seven or more peptides selected from the group consisting of peptides in SEQ ID NO: 1-6554. In some cases, cancer vaccines comprise eight or more peptides selected from the group consisting of peptides in SEQ ID NO: 1-6554. In some cases, cancer vaccines comprise nine or more peptides selected from the group consisting of peptides in SEQ ID NO: 1-6554. In some cases, cancer vaccines comprise ten or more peptides selected from the group consisting of peptides in SEQ ID NO: 1-6554. In some cases, cancer vaccines comprise 11, 12, 13, 14, 15, 16, 17, 18, 19, 20, or more peptides selected from the group consisting of peptides in SEQ ID NO: 1-6554. Adjuvants in vaccine compositions are often selected from the group consisting of ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan. Some such vaccine compositions are in some cases formulated for intramuscular or subcutaneous administration. Often, the vaccine composition further comprises a checkpoint inhibitor.

Also provided herein are vaccine compositions comprising a nucleic acid. Some such vaccine compositions often comprise a nucleic acid comprising at least one constitutive promoter, and encoding one or more peptides selected from the group consisting of SEQ ID NO: 1-6554. In some cases, cancer vaccines comprise one or more nucleic acids encoding two or more peptides selected from the group consisting of peptides in SEQ ID NO: 1-6554. In some cases, cancer vaccines comprise one or more nucleic acids encoding three or more peptides selected from the group consisting of peptides in SEQ ID NO: 1-6554. In some cases, cancer vaccines comprise one or more nucleic acids encoding four or more peptides selected from the group consisting of peptides in SEQ ID NO: 1-6554. In some cases, cancer vaccines comprise one or more nucleic acids encoding five or more peptides selected from the group consisting of peptides in SEQ ID NO: 1-6554. In some cases, cancer vaccines comprise one or more nucleic acids encoding six or more peptides selected from the group consisting of peptides in SEQ ID NO: 1-6554. In some cases, cancer vaccines comprise one or more nucleic acids encoding seven or more peptides selected from the group consisting of peptides in SEQ ID NO: 1-6554. In some cases, cancer vaccines comprise one or more nucleic acids encoding eight or more peptides selected from the group consisting of peptides in SEQ ID NO: 1-6554. In some cases, cancer vaccines comprise one or more nucleic acids encoding nine or more peptides selected from the group consisting of peptides in SEQ ID NO: 1-6554. In some cases, cancer vaccines comprise one or more nucleic acids encoding ten or more peptides selected from the group consisting of peptides in SEQ ID NO: 1-6554. In some cases, cancer vaccines comprise one or more nucleic acids encoding 11, 12, 13, 14, 15, 16, 17, 18, 19, 20, or more peptides selected from the group consisting of peptides in SEQ ID NO: 1-6554. Often, such vaccine compositions comprise an adjuvant. Adjuvants herein, in some cases, are selected from the group consisting of ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan. Such compositions are often formulated for intramuscular or subcutaneous administration. In some cases, the vaccine composition comprises a checkpoint inhibitor.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Certain novel features of the presently claimed invention are set forth with particularity in the appended claims. A better understanding of the features and advantages of the present invention will be obtained by reference to the following detailed description that sets forth non-limiting and non-exhaustive illustrative embodiments, in which the principles of the invention are utilized, and the accompanying drawings of which:

FIG. 1 shows a model demonstrating why in conventional methods all cancer vaccines must be custom or personal. Mice vaccinated with an irradiated tumor are only protected from challenge by that particular tumor. What was missed in this analysis was that a neo-antigen common between two tumors may have been missed by diluting its immune response. If one isolated the common neo-antigens and elicited the specific immune responses, there would have been protection. Data provided herein shows that there are common neo-antigens between different types of tumors.

FIG. 2 shows how an insertion or deletion in transcription of a MS would each produce a different FS peptide with examples of frameshift peptides produced by either an insertion or deletion in the MS in the Sec62 gene.

FIG. 3 shows a model for broad, frame-shift peptide production in tumor cells. Normal Cell: Errors in DNA replication are very low and repaired. Transcription error rates are higher but also rare as are mis-splicing in intron excision. Any frameshift (FS) transcript with a premature stop codon is the degraded as a target of nonsense-mediated decay (NMD). Aberrant proteins, including those with frame-shifts are largely eliminated by the protein quality control system. The net result is that very few FS peptides are presented on MHC I/II or escape the cell to be presented to the immune system in a normal cell. Cancer Cell: In general more RNA is produced and increased error rates are observed at the DNA, RNA, and protein levels. More errors are made in DNA replication, but only in dividing cells. Most DNA mutations are single nucleotide changes and encode low or non-immunogenic peptides. Transcription is also less accurate, especially through microsatellites (MS), producing insertions and deletions (Indels). Most transcribed genes with MSs in the coding region will have more FS transcripts. RNA splicing is also far less accurate in cancer cells, creating more FS transcripts from each out of frame splicing between exons form the same gene and different genes. The large increase of the FS transcripts from Indels of MS and mis-splicing overwhelms RNA quality control systems, such as NMD. Consequently, more truncated proteins with the FS peptide will be translated. These un-folded proteins, combining with aberrant proteins from other variants, overwhelms the protein quality control system leading to more FS peptides being presented on MHC I/II and mis-secreted or released from the cancer cell. The immune system can respond to these peptides.

FIG. 4 shows use of the FSP array can identify common FSPs in different cancers to form universal therapeutic and prophylactic cancer vaccines in for both human and dog. The FS peptide array screens common FSPs for a universal therapeutic vaccine. This figure shows the top 500 common FSPs (including FSP from both MS INDELs and mis-splicing) selected based positive rate in 6 cancers (17 serum samples per cancer, except cBC, n=12) and the positive of these peptides in 64 normal control samples. A red spot indicates the serum sample (Y axis) has positive antibody reactive to the FSP (X axis). The positive cut off value of each peptide was calculated by the average plus two fold of standard deviation of the normal controls' signal of that peptide. BC: breast cancer; GC: gastric cancer; GBM: glioblastoma; LC: lung cancer; PC: pancreatic cancer; cBC: children brain cancer.

FIG. 5 shows using the frameshift peptide (FSP) array can identify common FSPs in different cancers to form universal therapeutic and prophylactic cancer vaccines in for both human and dog. Figure shows top 500 FSPs (including FSP from both MS INDELs and mis-splicing) selected based positive rate in stage I patients of two cancers (BC 51: breast cancer, n=42; PC 51: pancreatic cancer, n=12) and 64 non-cancer normal controls.

FIG. 6 shows using the FSP array can identify common FSPs in different cancers to form universal therapeutic and prophylactic cancer vaccines in for both human and dog. The FSP array screens common FSPs for universal therapeutic dog cancer vaccine. This figure shows the top 500 common FSPs (including FSP from both MS INDELs and mis-splicing) in 8 different dog cancers (BC: breast cancer, n=12; FSA: fibrosarcoma, n=5; HSA: hemangiosarcoma, n=12; LC: lung cancer, n=12; MCT: master cell tumor, n=14; MEL: melanoma, n=12; OSA: osteosarcoma, n=17, STS: soft tissue sarcoma, n=12) and 81 non-cancer normal controls.

FIG. 7 shows using the FSP array can identify common FSPs in different cancers to form universal therapeutic and prophylactic cancer vaccines in for both human and dog. The FSP array screens common FSPs for a prophylactic or early therapeutic cancer vaccine. This figure shows top 500 FSPs (including FSP from both MS INDELs and mis-splicing) selected based positive rate in stage I patients of five cancers (FSA: fibrosarcoma, n=12; HSA: hemangiosarcoma, n=16; LSA: lymphoma, n=17; MCT: mast cell tumor, n=10; OSA: osteosarcoma, n=4) and 81 non-cancer normal controls.

FIG. 8A and FIG. 8B show examples of a set of FSPs that don't have positive antibody reactive in cancers on the 400K FSP array. Example of 500 FSPs on the 400K array that don't have positive reactive in cancers. FIG. 8A shows negative peptides in human cancers. Cancer n=151, Normal n=64. FIG. 8B shows negative peptides in dog cancers. Cancer n=159, Normal n=81.

FIG. 9 shows that the FSPs with negative antibody reactive don't have protection as cancer vaccine candidates, while the reactive FSPs have protection and its protection correlated with the vaccine elicited IgG reactive to the FSPs. FSP array analysis of the dog serum. 9 types of 116 dog cancer serum and 52 non-cancer serum were analyzed by the small FSP array. Each red spot indicates the serum has positive antibody reactive with this peptide.

FIG. 10A, FIG. 10B, FIG. 10C, and FIG. 10D show that the FSPs with negative antibody reactive don't have protection as cancer vaccine candidates, while the reactive FSPs have protection and its protection correlated with the vaccine elicited IgG reactive to the FSPs. Reactive FSPs showed protection in the mouse melanoma and breast cancer models. FIG. 10A shows reactive and non-reactive FSPs from the same microsatellite frameshift antigen of CIS. FIG. 10B shows 3 groups of mice were used: the control group was immunized with control plasmid, non-reactive FSPs pool and reactive FSP. All 3 groups received 2 rounds of genetic immunization with gene gun and peptide boost via subcutaneous injection. The Bl6F10 cell line was used for the melanoma model and the 4T1 cell line was used for breast cancer model. Each group had 10 mice. FIG. 10C shows reactive FSPs slowed tumor growth significantly compared to the non-reactive FSPs pool and the control group in the melanoma model (student's t-test, p-value <0.01), error bar represented mean±SEM. FIG. 10D shows the Reactive FSPs offered tumor protection in mouse breast cancer model as well. The tumor volume was significantly lower than control group and non-reactive FSPs pool group (student's t-test, p-value <0.05).

FIG. 11 shows that tumor volume was linearly correlated to antibody response against vaccinated reactive FSPs in the 4T1 breast cancer model. Tumor volume at the end point was used for analysis. The antibody response was measured by ELISA after PB2 and at the end point. R-COMP peptide was encoded in a plamsid and used as an internal control for genetic immunization. The p-value was calculated for linear fit of antibody response against tumor volume.

FIG. 12 shows that the negative FSPs have more pathogen epitopes than the highly active FSPs. This figure compares the negative FSPs with active FSPs in cancers. Select all negative FSPs and the FSPs with >10% positive rate in cancers from FIG. 9. A BLAST search against the Immune Epitope Database (IEDB) shows differential similarity with pathogen of peptides from these two sets FSPs. Each peptide was searched using BLAST against epitopes in the IEDB and all the epitopes having an e value less than 10,000 were recorded as the matches. The number of the matched epitopes of each peptide was put into boxplot and represented the pathogen similarity of that peptide. The difference in similarity was statistically calculated with T-test P<0.05.

FIG. 13 shows the sequence of the MS region in SEC62 homolog genes in paired DNA/cDNA samples from 4 human breast cancer cell lines: HTB26, CRL2315, CRL1504, CRL2326; 5 dog mast cell tumors; and 3 dog carcinomas. The sequence traces show the MSs have an A insertion in the cDNA. Examples of the sequence traces of MS region in SEC62 homolog genes in paired DNA/cDNA samples.

FIG. 14 shows homopolymer (repeat bp>7) MS Indel rate in EST database between different cancer and normal libraries. * indicate P<0.0001. Analysis of homopolymer (repeat bp>6) MS INDEL rate in EST database between different cancer and normal libraries. * indicate P<0.0001.

FIG. 15A and FIG. 15B show protection of the prophylactic immunization of FS antigens in a mouse tumor model. FIG. 15A shows tumor growth curve by three MS FS antigens immunization with 4T1-BALB/c tumor model. Mice (n=10 per group) were genetically immunized at 8 weeks of age, boosted twice genetically 3 weeks later with two days apart and another peptide boost 2 weeks later. Mice were challenged 2 week later by subcutaneous injection of 5×10³ 4 T1 cells. FIG. 15B shows ELISPOT analysis of the three MS FS antigen immunization. 3 mice were genetically immunized with pool of the three MS FA antigens and genetically boosted 6 weeks later. Immunized mice were challenged by 5×10³ 4 T1 cells 3 weeks later. Splenocytes were collected at 19 days after tumor challenge and pool three splenocytes for the assay. Error bars represent SD.

FIG. 16 A and FIG. 16B show the protection response in mice vaccinated with two pools of different FS antigens from the short (FIG. 16A) (8 homopolymer) and long (FIG. 16B) (10 to 18 homopolymer) MS regions. Tumor growth of curve the vaccine test of the two FSPs pool from short and long MS in 4T1-BALB/c tumor model. FIG. 16A shows tumor growth curve with short MS FSPs vaccine group. FIG. 16B shows tumor growth curve with long MS FSPs vaccine group.

FIG. 17 A and FIG. 17B shows the immunization of a pool of FSPs can also against the mouse ovarian cancer. FIG. 17A shows female C57BL/6 mice (n=10) were challenged intraperitoneally with ID8 (5×10⁵ cells/mice) at day 0 and then, immunized subcutaneously 3 times with pool of FSPs (RNA-FsNeoAg)(10 FS from MS INDEL and 3 FS from mis-splicing) or neo antigens from DNA mutations (DNA-NeoAg) at days 3, 6 and 78. Control group mice were injected PBS buffer. FIG. 17B shows the vaccine group received additionally a checkpoint inhibitor treatment (CPI) composed by antibody anti-PD-L1 and CTLA-4, six times divided in two sets. Mice were weighing once a week until reach endpoint.

FIG. 18 shows an exemplary vaccination schedule for testing a 21 FSP antigen pooled dog cancer vaccine for eliciting both antibody and T cell immune response in three healthy dogs and testing whether it is safe.

FIG. 19A, FIG. 19B, and FIG. 19C show a 21 FSP antigens pooled dog cancer vaccine can elicit both antibody and T cell immune response in three healthy dogs and it is safe. Immune response of the dog vaccine. FIG. 19A shows ELISA analysis of the IgG reactive to the vaccine. FIG. 19B and FIG. 19C show IFN-r ELISPOT analysis of the T cell response to the vaccine.

### DETAILED DESCRIPTION

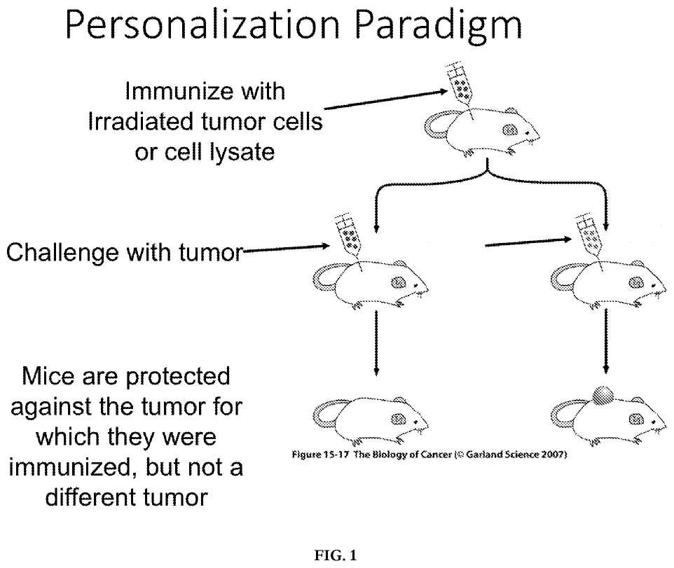

It has been thought that it is not possible to create a general therapeutic or preventative vaccine for tumors not caused by a specific infectious agent (Nature 491, 637 (29 Nov. 2012)). This was for at least the following reasons. First, in thirty years, efforts to create cancer vaccines from tumor associated self-antigens have not resulted in an effective cancer vaccine. This suggests that tumor associated self-antigens are not effective cancer vaccines. Rather, an effective cancer vaccine would comprise neo-antigens created by mutations found in tumors. Second, to date the sequencing of thousands of tumors (Vogelstein B, et al. 2013; Chang M T, et al. 2016) has revealed that almost all of the DNA encoded neo-antigens developed are unique to the tumor of each individual. This sequencing supports earlier mouse experiments that showed that cancer vaccines had to be personal (FIG. 1). Therefore, it was thought that it would not be possible to create a vaccine to anticipate a tumor (prophylactic vaccine) or to create a common, therapeutic vaccine of neo-antigens. Presented herein is data that demonstrates that this was not the case.

Described herein is the development of a feature in tumor cells that is the basis for recurrent production of neo-antigens leading to tumors of different types and from different individuals to have common neo-antigens. This finding enables the development of general therapeutic and prophylactic cancer vaccines that are a broadly protective in treating and preventing cancer.

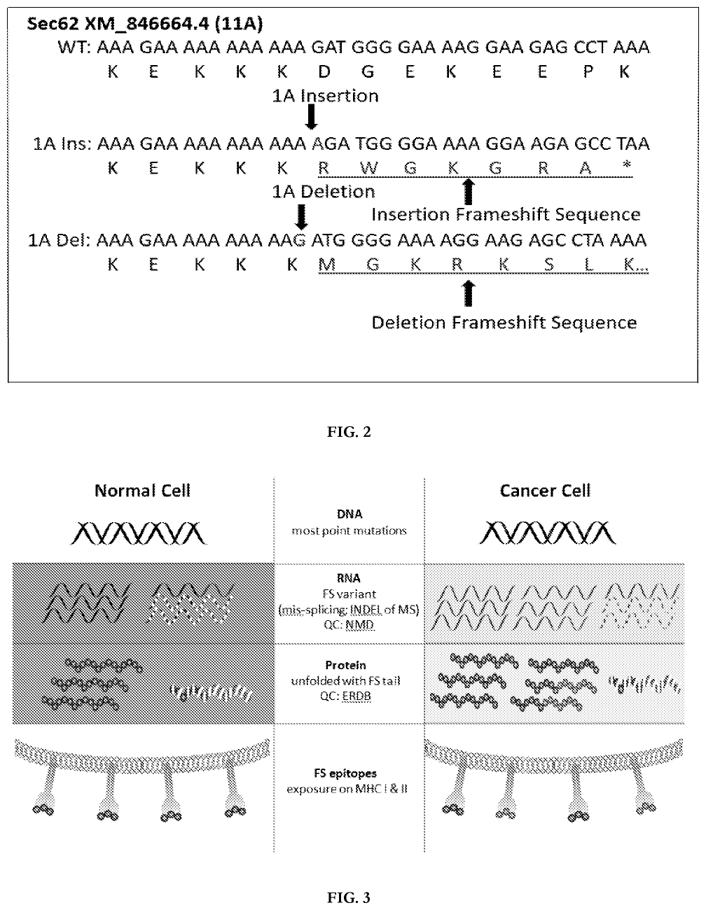

The novel development herein is that since more errors occur during RNA transcription than during DNA replication, transcription through microsatellites creates the best neo-antigens for vaccines. Tumor cells have high rates of error in transcription because the rapid growth of a tumor results in reduced quality control systems. Further, more errors in transcription are seen in tumors because tumor cells produce more RNA than non-malignant cells. Microsatellites (MS), repetitive sequences often found in coding regions, are the site of many transcriptional errors. RNA polymerase has difficulty staying in register through MS regions and, unlike for DNA replication, there is no proofreading correction in transcription. If the RNA polymerase inserts or deletes a single base while transcribing the MS, it will put the reading frame out, creating a premature stop codon and a C-terminal frameshift (FS) peptide on the protein (FIG. 2). This FS (there are two possible and predictable ones for each MS) will be a neo-antigen and highly immunogenic if the FS peptide is at least 8 amino acids long. Quality control systems in non-malignant cells would eliminate these rare events. However, quality control in tumor cells often fails, resulting in these FS peptides being translated and presented to the immune system.

The model presented herein (FIG. 3) explicitly predicts: 1) any gene containing a MS in the coding region will create FS antigens in tumors if expressed; 2) these FS antigens are predictable; 3) all will be expressed early in the tumor ontogeny; and 4) all FS antigens at least 8 aa long will be immunogenic and good vaccine components for a therapeutic or prophylactic vaccine and 5) highly expressed and essential genes will produce the most and stable amount of FSs. These predictions have been tested and found to be true—as shown in the Examples and drawings herein. The FS antigens are predictable, do not require development to distinguish useful vaccine candidates, and will be presented early in the development of a tumor. All FS of sufficient length (at least 8 aa), downstream of MS in coding regions of genes expressed in tumors can be used as vaccine components. This is a completely new concept in the area of cancer vaccines.

Frameshift Variants from Indels in Microsatellites

Methods herein, in some embodiments, comprise methods of designing a universal cancer vaccine. These methods comprise choosing peptides having FS variants. These FS variants are not “mutations” in that they are not in the DNA and therefore not heritable. Of importance, because the variants we identify are not heritable, it would be very difficult for a tumor to evolve away from the immune response to the FS variant vaccine. This is not the case for neo-antigen mutations, which are DNA mutations. FS variants, herein, are alterations in an mRNA caused by errors in transcription, causing an insertion or deletion (indel) of one or two nucleotides in the MS in the mRNA resulting in a change in the amino acids of the resulting protein that are encoded after MS variant. MS FS peptides are predicted by bioinformatics analysis of already available genome sequence data available for many species, including but not limited to humans and dogs.

Frameshifted polypeptide sequences predicted by inspection of DNA sequence resulting from indels in MS in coding sequences are assembled for further analysis.

FS variants are predicted based on MS location in the genome. As transcripts having an MS are more prone to transcription errors, FS polypeptides can be predicted to result from an insertion or a deletion of one or two basepairs. A prediction of the model is that essentially any MS of sufficient length (>6 homopolymers) would produce variant RNA at a significant level (>0.1%) and a FS from such an MS that was longer than 7 aa would be produced at a significant level and be immunogenic. Further, if the MS was in an essential gene, an oncogene and/or a highly expressed gene it would be difficult for a tumor to evolve away from the FS vaccine. We have applied these criteria and predict that every MS FS of this type would work as a cancer vaccine. This prediction was tested and met using a subset of MS FS in an animal model and statistical analysis of the results. Twenty-three MS FSs were tested in the mouse tumor models and all protected immunized mice. If the probability of a MS FS being protective was that predicted for DNA based neo-epitopes (3%) this result would be extremely unlikely (1×10⁻³⁵). Even if the probability of a FS being protective was 50%, the probability of 23/23 being protective is ˜1×10⁻⁷. Further, at 90% probability of being protective, the probability of 23/23 being protective would only be 0.09. Therefore, these data strongly support the unexpected and surprising finding that all MS FS of the type described will be effective as cancer vaccines.

Vaccines herein based on the MS FSs may also be useful in eliminating aberrant cells that are not tumors. For example, cells playing a role in diabetes, Alzheimer's disease, aging, chronic infections, or other diseases. Any cell that met the criteria depicted in FIG. 3 under “cancer cell” could be eliminated by the vaccine disclosed herein.

Peptides from Frameshift Variants

Microsatellite derived, frameshifted polypeptide sequences determined by genomic sequence inspection can be further analyzed to determine the relative suitability for vaccine composition. It is well established that the longer the MS the more probable are errors in transcription through it. Therefore, we choose MSs longer than 6 nucleotides. For example, an MS consisting of AAAAAAA or longer. In order for the FS peptides to be immunoreactive they should be long enough to bind an MHC protein or an antibody. For this reason we choose FS peptides that are at least 8 aa long. On the other hand, a very long FS peptide (e.g., >100 aa) is more likely to have a function and more likely to be used in some cell type, raising the possibility of an autoimmune reaction. These longer FS can be inspected relative to the codon usage and amino acid composition to further determine which ones might encode a functional unit. These long sequences can also be searched in BLAST against all proteomes to look for similarities. Long FS that are determined to resemble real proteins are excluded by these criteria.

In the human genome, there are 6,282 genes containing MS regions (>6 nt homopolymer) with a total of 8,617 MS regions, which could generate total 17,234 different FS transcripts by insertion or deletion during the gene transcription. These FS transcripts encode total 10,067 FS peptides longer than 7 aa which includes 2,970 FS peptides encoded by an essential gene, cancer driver gene or top 20% high expression genes (SEQ ID NO: 40-2829). Peptides having the sequences SEQ ID NO: 2829-5855 are the corresponding peptides for dogs.

### Methods of Designing a Universal Vaccine

A universal vaccine for administration prophylactically or therapeutically can be comprised of any of the MS FS antigens in SEQ ID NO: 1-6554. The number of MS FS antigens in the vaccine could be restricted by applying any of the criteria in SEQ ID NO: 1-6554. For example, one could choose all MS FS in essential genes that are greater than 30 aa long. Or one could choose all MS FS where the MS is longer than 6 nucleotides. Or one could choose a combination of criteria. One could also use the arrays of FS peptides to screen sera to determine the most often presented FS in a set of cancer types or even all cancer types. One remarkable aspect of this development is that all the MS FS in the SEQ ID NO: 1-6554 will work as vaccines. Although any FS listed will work as a vaccine, a subset could be chosen based on biological criteria, manufacturing considerations, etc.

It is also possible, but probably not necessary or as useful as the criteria on SEQ ID NO: 1-6554, to rank the MS FS antigens by various experimental methods as described below. One would need to apply these methods to a large number of people with cancer to determine the antigens with highest average reactivities. These types of measures of people with cancer may not apply to evaluating components for a prophylactic vaccine.

Binding to MHC is required for T cell activity and can be determined by binding assays. Alternatively, in silico methods of MHC binding are used to predict binding of a peptide to a MHC subtype.

Data of peptides binding to MHC subtype molecules are used to develop binding prediction algorithms. These algorithms calculate scoring matrices that quantify the contribution of each residue in a fixed-length peptide to binding to an MHC molecule. Algorithms predict binding of a peptide to class I MHC or class II MHC. Algorithms to predict class I MHC binding include but are not limited to Artificial neural network (ANN), Stabilized matrix method (SMM), SMM with a Peptide:MHC Binding Energy Covariance matrix (SMMPMBEC), Scoring Matrices derived from Combinatorial Peptide Libraries (Comblib_Sidney2008), Consensus, NetMHCpan, NetMHCcons and PickPocket. Algorithms to predict class II MHC binding, include but are not limited to Consensus method, Combinatorial library, NN-align (netMHCII-2.2), SMM-align (netMHCII-1.1), Sturniolo, and NetMHCIIpan. The entire population of FS polypeptides is then scanned using one or more of the above algorithms for peptides binding to an MHC subtype molecule with a predicted affinity of IC50 <500 nM.

### Identifying Frameshift Peptide Antibody Response

Candidate frameshift peptides (FSP) for universal cancer vaccines, in some embodiments, are screened for antibody reactivity. In some embodiments, candidate FSPs are selected for a universal cancer vaccine based on antibody reactivity to the candidate FSP. Antibody reactivity is determined using an assay for antibody binding to a peptide. In some embodiments, peptides for antibody screening are bound to a substrate, such as a plate, a glass slide, a bead, or other substrate. Assays for antibody binding include but are not limited to ELISA, radio-immuno assay, western blot, surface plasmon resonance, immunostaining, immunoprecipitation, mass spectrometry, phage display, ELISPOT, flow cytometry, cytometric bead array, immunohistochemistry, high density array, microarray, delayed-type hypersensitivity (DTH), and combinations thereof.

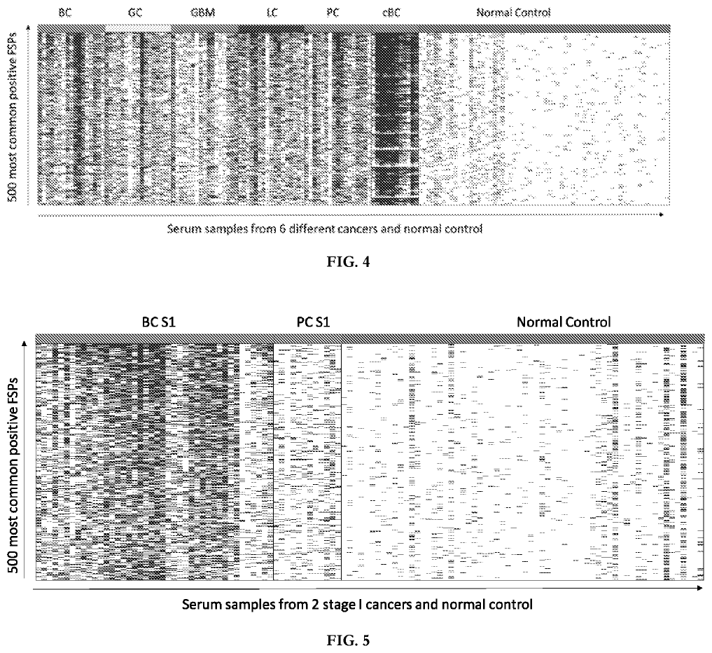

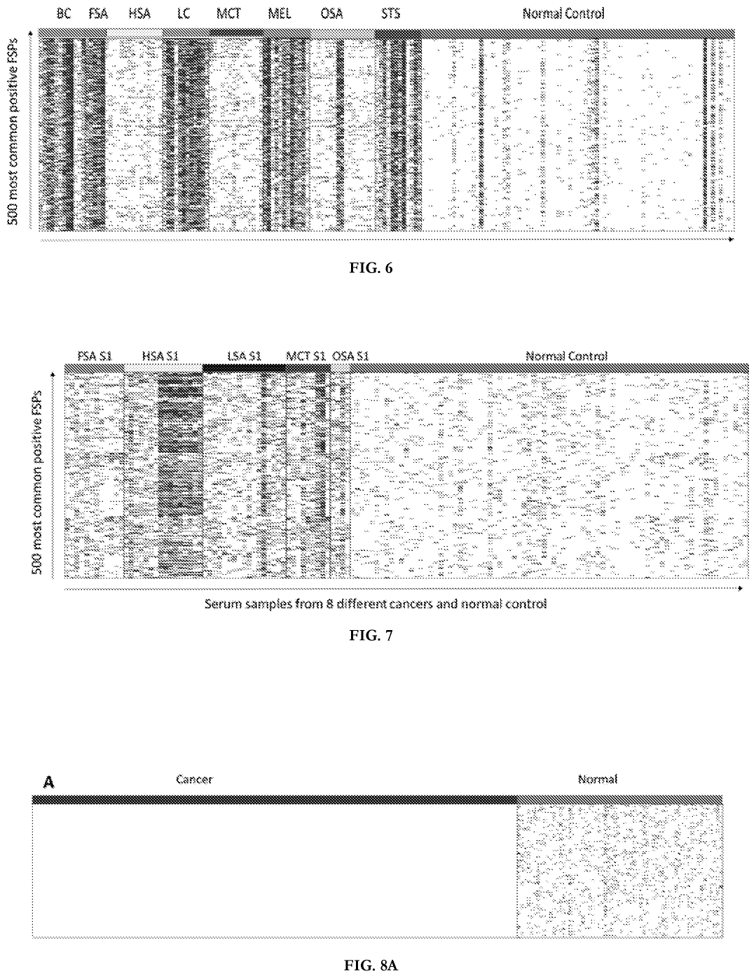

In an exemplary embodiment, a blood sample is obtained from an individual. The blood sample is diluted in an appropriate buffer and applied to a peptide microarray spotted with vaccine candidate FS peptides. The diluted blood sample is incubated with the peptide microarray overnight and the peptide microarray is then washed and exposed to a secondary antibody that binds to human IgG bound to the microarray. The secondary antibody is conjugated with Alexa Fluor 488 and the microarray is analyzed for fluorescence at each peptide spot. The peptides bound to the individual's antibodies are deemed immunoreactive and selected as a component of a universal cancer vaccine. Arrays of FS peptides meeting all the criteria above have been developed. These contain all the FS from MS as well as all from exon mis-splicing. There are 220,000 such peptides represented in 400,000 spots. FIG. 4 shows the results of screening sera of people with 6 different cancers, including children brain cancer, to identify common peptides for a universal therapeutic. FIG. 5 shows how these peptides can be grouped to select ones in common for early stages of cancer. FIG. 6 and FIG. 7 show the same comparisons for dog cancers.

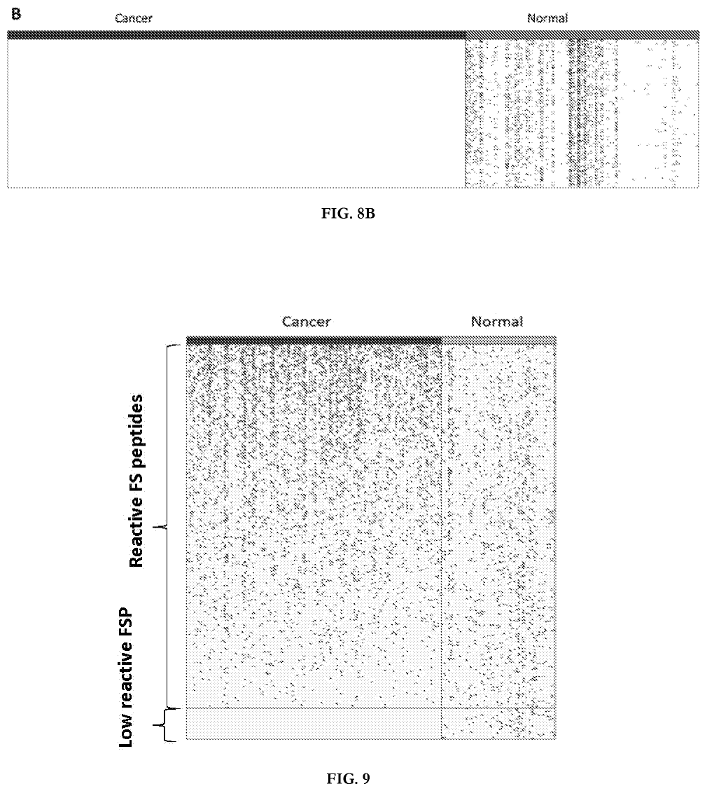

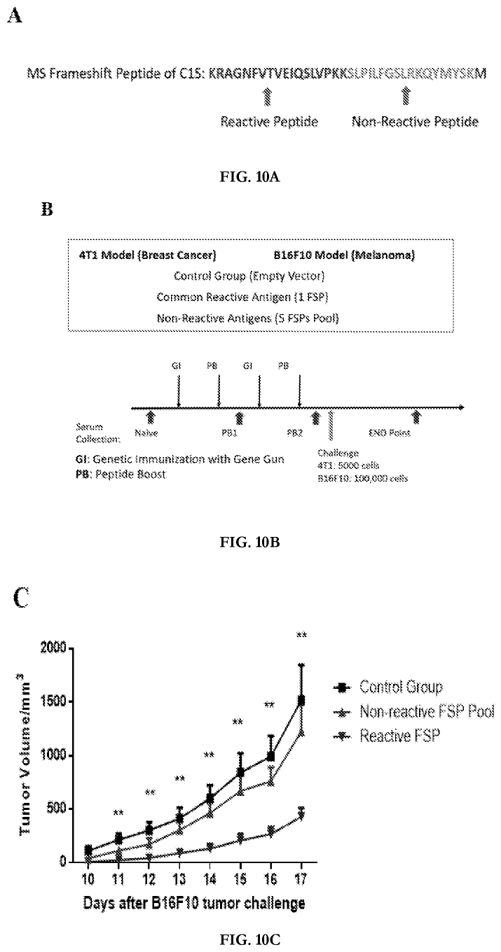

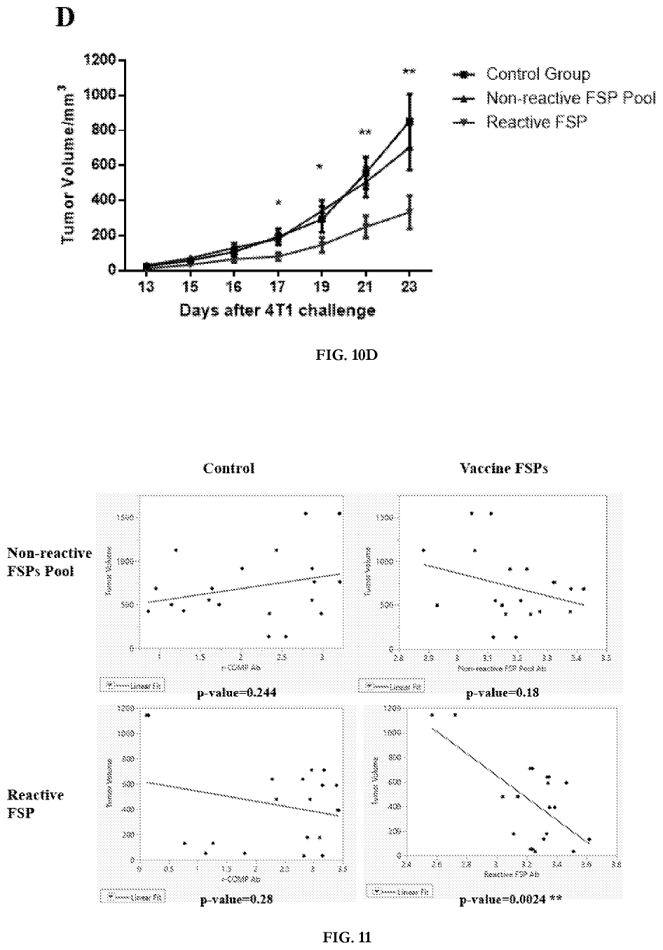

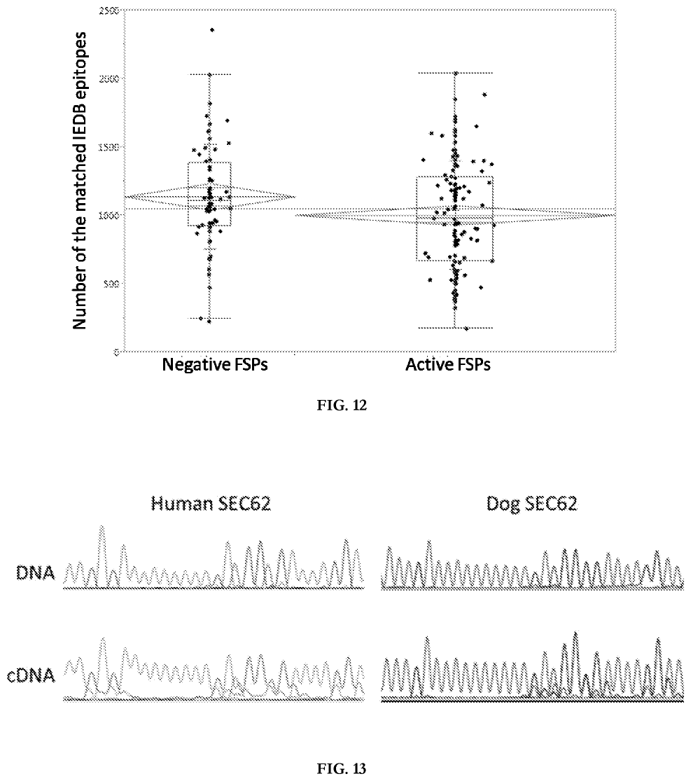

These arrays encompassing all the possible MS FS arrays can be used to screen a large number of samples for vaccine candidates. It was found that a portion of FS peptides don't have positive IgG reactive in people or dogs with cancer (FIG. 8). Mouse homologs of some negative FS peptides were tested and it was determined that they do not convey protection while FS from the cancer reactive sector always convey protection (FIG. 9 and FIG. 10). It was also found that the tumor volume of each immunized mouse was correlated with the IgG reactivity to the immunized highly reactive FSPs (FIG. 11). CD4+ T help cell is involved in IgG reactivity. This suggests these reactive FSPs elicited CD4+ T cell response, which is also playing an important role in anti-tumor immune response. Therefore, these comprehensive arrays can be used to pick protective from non-protective FS antigens. The sequence of these negative FSPs from a small FSP array was analyzed and compared with the highly reactive FSPs. The analysis showed that these negative FSPs are more homologous to epitopes from pathogen epitope (FIG. 12). It is possible that the frequently contacting different pathogen in regular life induces the tolerance to these epitopes and consequently, decreases the effective immune responses to the FSPs containing these epitopes.

Accordingly, in some embodiments, selecting candidate FSPs for a cancer vaccine herein, comprises determining the sequence similarity to known pathogen epitopes and selecting FSPs that do not show sequence similarity to known pathogen epitopes. Known pathogen epitopes comprise peptides catalogued in the Immune Epitope Database (IEDB, available on the internet at iedb.org). In some cases, candidate FSPs are searched via a BLAST algorithm against pathogen epitopes in the IEDB. In some embodiments, a candidate FSP is selected for having a BLAST e value less than 10,000.

In another exemplary embodiment, an individual with cancer is given an intradermal injection of each candidate peptide in a pre-determined pattern on the patient's back to measure delayed-type hypersensitivity (DTH) response. Erythema and induration are measured at 24, 48, and 72 hours. The peptides which elicit a DTH response are deemed immunoreactive. Peptides that are broadly reactive in many cancer patients can be selected as components of a universal cancer vaccine.

### Identifying Frameshift Peptide T Cell Response

In some embodiments, candidate frameshift (FS) peptides are screened for T cell activity. T cell activity is determined using a T cell assay measuring proliferation, cytokine secretion, cytotoxicity, or degranulation in response to a FS peptide bound to an antigen presenting cell. T cell assays include but are not limited to proliferation assay, 3H-thymidine assay, BrdU assay, CFSE assay, cytokine secretion assay, ELISA assay, ELISPOT assay, intracellular staining assay, quantitative rtPCR assay, cytometric bead array assay, MHC-tetramer binding assay, cytotoxicity assay, 51-chromium assay, degranulation assay, granulysin assay, granzyme A assay, granzyme B assay, and perforin assay.

In an exemplary embodiment, a blood sample is obtained from an individual. PBMCs are isolated from the blood sample and the PBMCs are cultured to expand T cells in the sample and the T cells are incubated in culture media containing one or more candidate peptides for a cytokine release assay. The production of IFN-γ is analyzed in ELISPOT assays. Flat-bottom 96-well nitrocellulose plates are prepared and coated with either anti-human IFN-γ. Cells were then incubated at a density of 1×10⁵/well either with peptide pools or individual peptides (10 μg/ml), PHA (10 μg/ml), or medium (containing 1% DMSO corresponding to the percentage of DMSO in the pools/peptides) as a control. After 24 hours, cells are removed, and plates are incubated with HRP-conjugated anti-human IFN-γ Ab (Clone 7-B6-1, Mabtech) at 37° C. After 2 hours, spots corresponding to the HRP-conjugated Ab (IFN-γ) are developed with 3-amino-9-ethylcarvazole solution (Sigma-Aldrich, St. Louis, Mo.). Spots are counted by computer-assisted image analysis (Zeiss, KS-ELISPOT reader, Munich, Germany). Each assay is performed in triplicate. The level of statistical significance is determined with a Student's t-test using the mean of triplicate values of the response against relevant pools or individual peptides versus the response against the DMSO control. Criteria for peptide pool positivity are 100 spot-forming cells (SFCs)/10⁶ PBMC, p≤0.05 and a stimulation index (SI) ≥2, while criteria for individual peptide positivity are ≥20 SFC/10⁶ PBMC, p≤0.05, and a SI ≥2.

The peptide arrays can include control sequences that match epitopes of well characterized monoclonal antibodies (mAbs). Binding patterns to control sequences and to library peptides can be measured to qualify the arrays and the assay process. Additionally, inter wafer signal precision can be determined by testing sample replicates e.g. plasma samples, on arrays from different wafers and calculating the coefficients of variation (CV) for all library peptides. Precision of the measurements of binding signals can be determined as an aggregate of the inter-array, inter-slide, inter-wafer and inter-day variations made on arrays synthesized on wafers of the same batch (within wafer batches). Additionally, precision of measurements can be determined for arrays on wafers of different batches (between wafer batches). In some embodiments, measurements of binding signals can be made within and/or between wafer batches with a precision varying less than 5%, less than 10%, less than 15%, less than 20%, less than 25%, or less than 30%.

The technologies disclosed herein include a photolithographic array synthesis platform that merges semiconductor manufacturing processes and combinatorial chemical synthesis to produce array-based libraries on silicon wafers. By utilizing the tremendous advancements in photolithographic feature patterning, the array synthesis platform is highly-scalable and capable of producing combinatorial peptide libraries with 40 million features on an 8-inch wafer. Photolithographic array synthesis is performed using semiconductor wafer production equipment in a class 10,000 cleanroom to achieve high reproducibility. When the wafer is diced into standard microscope slide dimensions, each slide contains more than 3 million distinct chemical entities.

In some embodiments, arrays with peptide libraries produced by photolithographic technologies disclosed herein are used for immune-based assays. Using a patient's antibody repertoire from a biological sample bound to the arrays, a fluorescence binding profile image of the bound array provides sufficient information to classify which peptides are reactive with an antibody from the patient. FIG. 4, FIG. 5, FIG. 6, and FIG. 7 display using such FS arrays to construct universal prophylactic or therapeutic vaccines.

These platforms of screening MS FS can be applied to the peptides in SEQ ID NO: 1-6554.

The following peptides include all MS FS that have been tested in a mouse tumor model and which were protective. Mus 864-P, C1S, NM_173864.2, 7_A, Del, human homology, dog homology, KKRVGSFVTMEIPSPVLKKALPILIGSPIRQNTCLKMW (SEQ ID NO: 1); Mus942, Prdm2, NM 001081355.3, 8_A, In, human homology, KKKSAGTESDPSEEQICEAEGRPEGHFRGVLTYLPLL (SEQ ID NO: 2); Mus956, Vasp, NM_009499.3, 8_C, Del, human homology, dog homology, PPHPQVCPPQGYLGQVMEQGQPHPLHPHSLQHRAPIVGVPGPQAWLLPLLEPNSGK (SEQ ID NO: 3); Mus392, 6030458C11Rik, NM_029998.3, 8_U, Del, human homology, dog homology, FFLSKTPSKKCSLRMNTKFYRSFTSLKSLIVTFLRMVWWMLLRLEPISWKI (SEQ ID NO: 4); Mus413, Rfc3, NM_027009.2, 10_A, Del, human homology, KKLKSAPLQATTTLKLIPVMRGTATEL (SEQ ID NO: 5); Mus274, Chd2, NM_001081345.2, 10_A, In, human homology, dog homology, KKVLPNLPSQSSTF (SEQ ID NO: 6); Mus694, Slc35f5, NM_028787.4, 10_U, In, human homology, dog homology, FFLLCVVFGKFVIPRSTFRHTGCHSEYFVFNFWTFYFNPCSCISE (SEQ ID NO: 7); Mus281, Ccdc112, NM_001160399.1, 11_A, In, human homology, dog homology, KKRVYSKLENQKAAKEGGNTQVKRKGGHRASAFSKQSRR (SEQ ID NO: 8); Mus263, Plcl1, NM_001114663.1, 8_A, In, KKSCPRYDPTLISLLYQCVS (SEQ ID NO: 9); Mus671, Enthd1, NM_001163189.1, 8_A, In, KKKHIQGQAEAGAVPGRALACWDLSAPVLPFTWDEGVEIYRGPNTVVLL (SEQ ID NO: 10); Mus334, Pcdh18, NM_130448.3, 8_A, In, KKRASPLLGRTPLATRIRETLAHHLCYQK (SEQ ID NO: 11); Mus051, Supt16, NM_033618.3, 8_A, In, KKIPSWGRSFYCGNVLPSYHSEWWQL (SEQ ID NO: 12); Mus587, Trio, NM_001081302.1, 8_A, In, KKKGLFHARISVQEQYQGELPLLGGKCGERSL (SEQ ID NO: 13); Mus385, Olfr1246, NM_146792.2, 8_U, Del, FLFPAFSCMPDLFITFLVTNTLLYFIQFSLPC (SEQ ID NO: 14); Mus023, O1fr139, NM_147003.1, 8_U, In, FPPPGRCGLSSLDSHGL (SEQ ID NO: 15); Mus927, Zfp91, NM_053009.3, 9_A, Del, ICMSPPLLWATLQAPETTSAACKASYRPEGLYL (SEQ ID NO: 16); Mus255, Adam24, NM_010086.4, 9 A, In, YFSCDKRCIKHYAGNKSLLTFSGY (SEQ ID NO: 17); Mus518, Il1f9, NM_153511.3, 10_A, Del, TLCMEVMLRWNTRELGYLYLQLCFLNTHFLHTSQEEKLLTLGRFLTWTSRCGSFVIRPL (SEQ ID NO: 18); Mus487, Fam71a, NM_001109759.1, 10_A, In, KKRQKKIRPPKKKRSIQGQRQKPPRDHRCECDQLFCFFWWGGNP (SEQ ID NO: 19); Mus951, Nemf, NM_025441.3, 11_A, In, KKKAKEQAAAEAAEEQAAACRCGSQPVSLCQCQKIL (SEQ ID NO: 20); Mus461, Calr4, NM_001033226.4, 13_U, In, FFFFSLSLSFLHRWMDKTVGTI (SEQ ID NO: 21); Mus528, Gm5464, NM_001034881.3, 14_U, Del, FFVTAGYQEEEMEASACGAKGPGLAPWPPSWLALQDSLLCVVVALADLRRKSCC (SEQ ID NO: 22); and Mus414, Ankrd45, NM_028664.1, 18_U, In, FFFGVNGARRNSRIGEFRKVTIFLTARV (SEQ ID NO: 23).

The following peptides include the human homologs of MS FS tested in the mouse tumor model. Mus 864-P, C1S, NM_001346850.1, 7_A, Del, KKRAGNFAIMEIQCPALRKTLPILFGSLRRQNMSLEMWCR (SEQ ID NO: 24); Mus942, Prdm2, NM_001007257.2, 8_A, In, KKSASTESNSSEKQICKAEGRLEKCL (SEQ ID NO: 25); Mus956, Vasp, NM_003370.3, 8_C, Del, PPHPQVCPLRGSQLQRTEQGEDHPLHPLSRQHRALVVGELGPQAWPQLLLEPNSGKSASRRRPQGG PQPPKLRVVEAEVGDSWKR (SEQ ID NO: 26); Mus392, 6030458C11Rik, NM_018356.2, 8_T, Del, FFQSRIPSKKCSLRKSTKSYKSCTNLRNLAPT (SEQ ID NO: 27); Mus413, Rfc3, NM_002915.3, 10_A, Del, KKLKLAPLQVTTTLKLILVMLEIVTE (SEQ ID NO: 28); Mus274, Chd2, NM_001271.3, 11_A, In, KKILLNLSCQSCTF (SEQ ID NO: 29); Mus694, Slc35f5, NM_001330314.1, 10_T, In, FFLLCVVFGKFVISRSTFRHTSCYS (SEQ ID NO: 30); and Mus281, Ccdc112, NM_152549.2, 7_A, In, KKRVNSDLEN (SEQ ID NO: 31).

The following peptides include the dog homologs of MS FS tested in the mouse tumor model. Mus 864-P, C1S, XM_005637210.2, 7_A, Del, KKRAGNFVTVEIQSLVPKKSLPILFGSLRKQYMYSKM (SEQ ID NO: 32); Mus942, Prdm2, XM_005617980.2, No MS (SEQ ID NO: 33); Mus956, Vasp, NM_001003256.1, 8_C, Del, PPHPLVCPSQGALRQGTEQGEAHPLRPLSPQHKAPVVGEQGPPVLQQPLPEPNSGKSASKRRPQGGP QSLKQRALEARAGG (SEQ ID NO: 34); Mus392, 6030458C11Rik, XM_850372.4, 8_T, Del, FFQSRIPSKKCSLRKSTKSYKSCTSLKSLVPT (SEQ ID NO: 35); Mus413, Rfc3, XM_534500.5, No MS (SEQ ID NO: 36); Mus274, Chd2, XM_005618324.2, 11_A, In, KKILLNLSSESCTF (SEQ ID NO: 37); Mus694, Slc35f5, XM_533330.4, 10_T, In, FFLLCVVSGKFVVSRSTFRHTGCYS (SEQ ID NO: 38); and Mus281, Ccdc112, XM_005626355.1, 7_A, In, KKRVYSELENQKAAKEGGNFQ (SEQ ID NO: 39).

The following peptides are all the MS FSs in humans meeting the criteria of >6 homopolymers, a FS peptide length >7 amino acids long and either a cancer driver gene, an essential gene, or a highly expressed gene. CNOT1, Del, U_13, 12, essential, high expression, FFFFCYILNTMF (SEQ ID NO: 40); CNOT1, In, U_13, 13, essential, high expression, FFFFSVIFSTRCL (SEQ ID NO: 41); RNPC3, Del, A_12, 20, essential, KKKGLMTLSKMIKKKKNLVI (SEQ ID NO: 42); MIS18BP1, In, A_11, 10, essential, KKKIAAYLPM (SEQ ID NO: 43); CHD2, In, A_11, 15, high expression, KKKILLNLSCQSCTF (SEQ ID NO: 45); RNF145, Del, A_11, 15, high expression, KKKTTWLQRRNWRQC (SEQ ID NO: 46); RNF145, Del, A_11, 19, high expression, KKKRKKKTTWLQRRNWRQC (SEQ ID NO: 47); MIS18BP1, Del, A_11, 25, essential, KKNCSIPTYVKKRKTTNHSSQMTVH (SEQ ID NO: 48); TAF1B, Del, A_11, 28, essential, KKKTILKKAGIGMCVKVSSIFFINKQKP (SEQ ID NO: 49); MARCKS, In, A_11, 31, high expression, KKKKEALFLQEVFQAERLLLQEEQEGGWRRR (SEQ ID NO: 50); RNF145, In, A_11, 46, high expression, KKKKQHGCKGETGGSVKCGPEGAKHHAVGCPVQMGCQLLFPADPKK (SEQ ID NO: 51); RNF145, In, A_11, 50, high expression, KKKKEKKKQHGCKGETGGSVKCGPEGAKHHAVGCPVQMGCQLLFPADPKK (SEQ ID NO: 52); RNASEH2B, In, A_10, 8, essential, KKKNWKGL (SEQ ID NO: 53); UVRAG, Del, A_10, 8, essential, KKKVNACS (SEQ ID NO: 54); GART, Del, A_10, 8, essential, KKRPEWLS (SEQ ID NO: 55); WNK1, Del, A_10, 12, essential, high expression, KKSRKRAVSNSR (SEQ ID NO: 56); ABCF1, In, A_10, 16, essential, high expression, KKKARYPKRQAEEGCG (SEQ ID NO: 57); TFAM, In, A_10, 17, essential, high expression, KKKRKAEDCKGKLEKSV (SEQ ID NO: 58); TRMT10C, In, A_10, 17, essential, KKKIFKIFIYEGKSEKS (SEQ ID NO: 59); TFAM, In, A_10, 18, essential, high expression, KKKRVNTAWKTKKTSFSL (SEQ ID NO: 60); PARP14, In, A_10, 22, high expression, KKKLLIHLPPSHWDRKCQTTPR (SEQ ID NO: 61); SEC63, In, A_10, 26, essential, high expression, KKKTYTCAITTVKATETKAGKWSRWE (SEQ ID NO: 62); PARP14, Del, A_10, 27, high expression, KKITHPFASQPLGQEMPNNTQIRLLKP (SEQ ID NO: 63); RFC3, Del, A_10, 27, essential, KKKLKLAPLQVTTTLKLILVMLEIVTE (SEQ ID NO: 64); GART, In, A_10, 28, essential, KKKGQSGCLNIWNRIEPASTYRQYSGTK (SEQ ID NO: 65); WNK1, In, A_10, 29, essential, high expression, KKKAGREQSQTAGRTIQCFPDRNQAAPFC (SEQ ID NO: 66); SEC63, Del, A_10, 29, essential, high expression, KKNLHLCYYHSQSNRNKSRQMESLGMKLQ (SEQ ID NO: 67); TGFBR2, Del, A_10, 36, driver, high expression, KKSLVRLSSCVPVALMSAMTTSSSQKNITPAILTCC (SEQ ID NO: 68); ABCF1, Del, A_10, 61, essential, high expression, KKSEIPEKAGGRRMWMMMEKRKSSWSVLRSSQCQPVMRRMKYPPQNPAEGRKPRVVMFLQP (SEQ ID NO: 69); PPP1R12B, Del, U_10, 8, high expression, FFSMKIQS (SEQ ID NO: 70); SLC35F5, Del, U_10, 24, high expression, FFFALCGFWQICHIKKHFQTHKLL (SEQ ID NO: 71); SLC35F5, In, U_10, 26, high expression, FFFLLCVVFGKFVISRSTFRHTSCYS (SEQ ID NO: 72); ZNF644, Del, A_9, 8, high expression, KKESEKWM (SEQ ID NO: 73); KIF21A, Del, A_9, 8, high expression, KKFQSLLL (SEQ ID NO: 74); PRDM2, In, A_9, 8, high expression, KKKSFSFI (SEQ ID NO: 75); EEA1, In, A_9, 8, high expression, KKNPETRF (SEQ ID NO: 76); SFSWAP, In, A_9, 8, essential, KKKIRSQL (SEQ ID NO: 77); EIF2B3, In, A_9, 8, essential, KKKSSGAA (SEQ ID NO: 78); ARV1, Del, A_9, 9, high expression, KKSPTSFCC (SEQ ID NO: 79); MED8, Del, A_9, 9, essential, KKMPTCSSG (SEQ ID NO: 80); TCF7L2, Del, A_9, 9, driver, KKVRSLHTR (SEQ ID NO: 81); HDAC2, In, A_9, 10, high expression, KKKSLLLLRR (SEQ ID NO: 82); MED8, In, A_9, 10, essential, KKKCPPAALV (SEQ ID NO: 83); RBBP8, In, A_9, 10, essential, KKKTSENTPF (SEQ ID NO: 85); TRMT6, Del, A_9, 10, essential, KKNMKPSLLL (SEQ ID NO: 86); TCF7L2, In, A_9, 10, driver, KKKVRSLHTR (SEQ ID NO: 87); ANO10, Del, A_9, 11, high expression, KKKMEITMMIS (SEQ ID NO: 89); ANO10, In, A_9, 11, high expression, KKKRWRCPVVV (SEQ ID NO: 90); ZMYM4, In, A_9, 11, high expression, KKKTYCYHKYK (SEQ ID NO: 91); CEP57, In, A_9, 11, essential, KKNARVGSKTP (SEQ ID NO: 92); SMG7, In, A_9, 11, essential, KKNEAFSHGAI (SEQ ID NO: 93); MIS18BP1, Del, A_9, 11, essential, KKVPSASPWGL (SEQ ID NO: 94); RBPJ, In, A_9, 12, essential, high expression, KKKRTNGTRWLF (SEQ ID NO: 95); CCDC43, In, A_9, 12, high expression, KKKDTERGAKAI (SEQ ID NO: 96); ACBD3, In, A_9, 12, high expression, KKKEGGRGAKAA (SEQ ID NO: 97); ZNF292, Del, A_9, 12, high expression, KKVLTKQKVLYK (SEQ ID NO: 98); GTF3C3, In, A_9, 12, essential, KKNFRRRHLRRE (SEQ ID NO: 99); VCP, In, A_9, 13, essential, high expression, KKKCVHHWRYQPA (SEQ ID NO: 100); CEBPZ, Del, A_9, 13, essential, high expression, KKKMLNQKCLAPF (SEQ ID NO: 101); TNKS2, In, A_9, 13, high expression, KKKQGWKYSFGSC (SEQ ID NO: 102); KMT2C, In, A_9, 13, driver, KKKGTRKQNSGSL (SEQ ID NO: 103); DYNC1I2, Del, A_9, 14, essential, high expression, KKGEKLKHCFKAWG (SEQ ID NO: 104); ZFR, In, A_9, 14, essential, high expression, KKKSKSRFASRSKA (SEQ ID NO: 105); ERCC5, In, A_9, 14, driver, high expression, KKKITDIATHPWLS (SEQ ID NO: 106); PDS5B, In, A_9, 14, high expression, KKKCPCRTLQTSSY (SEQ ID NO: 107); ANO10, Del, A_9, 14, high expression, KKKMEVPSCCLDHC (SEQ ID NO: 108); CNTRL, In, A_9, 14, driver, KKNKCSTNSTIRTA (SEQ ID NO: 109); PPP1R10, In, A_9, 15, essential, high expression, KKKSTVTYGCQAKPL (SEQ ID NO: 110); RAD50, Del, A_9, 15, essential, high expression, KKRKSGVMKCWDLCP (SEQ ID NO: 111); UPF3A, Del, A_9, 15, high expression, KKKRQINRRKLQRKK (SEQ ID NO: 112); SCAPER, In, A_9, 15, high expression, KKKSQKDKSPDELQG (SEQ ID NO: 113); KIF21A, In, A_9, 15, high expression, KKNSRAFSCNKEKGI (SEQ ID NO: 114); GINS1, Del, A_9, 15, essential, KKIASTFYLDGNVSS (SEQ ID NO: 116); ARV1, In, A_9, 16, high expression, KKKAQLHFAAESIIII (SEQ ID NO: 117); RTN4, In, A_9, 16, high expression, KKNRRKEGPNSNREEY (SEQ ID NO: 118); CHD2, Del, A_9, 16, high expression, KKNRRRKRTRRTRRNK (SEQ ID NO: 119); CIR1, In, A_9, 16, high expression, KKNTKEEKKEKQVFRA (SEQ ID NO: 120); MIS18BP1, In, A_9, 16, essential, KKKSHQQVHGDFRKYI (SEQ ID NO: 121); SEC31A, Del, A_9, 17, high expression, KKLMLLRLNLRKMCGPF (SEQ ID NO: 122); BLM, Del, A_9, 17, driver, essential, KKMKALISQEMFSQALL (SEQ ID NO: 123); HOXA11, Del, A_9, 17, driver, KKLTETVYSTTQQIHSS (SEQ ID NO: 124); BPTF, Del, A_9, 18, essential, high expression, KKKRRNRKKKKRCSKRHG (SEQ ID NO: 125); SPAG9, In, A_9, 18, high expression, KKKVKHLAVFQPTFQLLK (SEQ ID NO: 126); TCF25, Del, A_9, 18, high expression, KKNRKTRKAAREKHRKTD (SEQ ID NO: 127); CHD2, In, A_9, 19, high expression, KKKTEEEREQGEQGETNEF (SEQ ID NO: 128); ZNF644, In, A_9, 20, high expression, KKKKAKNGCKQDNSLYRGLL (SEQ ID NO: 129); CHEK1, In, A_9, 20, essential, KKNIPQPLEKNRFCSSSSAA (SEQ ID NO: 130); DYNC1I2, In, A_9, 21, essential, high expression, KKKRNRPEEGSCCSCARRIRS (SEQ ID NO: 131); LMAN1, In, A_9, 22, high expression, KKKRGIPEGPPRPPRAACGGNI (SEQ ID NO: 132); ANO10, In, A_9, 22, high expression, KKKRWSEKHHVWHRDSGVSTA (SEQ ID NO: 133); HDAC2, Del, A_9, 22, high expression, KKKSATTTTVILEIIIMDRVIP (SEQ ID NO: 134); SPAG9, In, A_9, 22, high expression, KKKVKHLAVCANSFQPTFQLLK (SEQ ID NO: 135); RBPJ, Del, A_9, 23, essential, high expression, KKKNKWNAMVVLNKSLNRVHLLG (SEQ ID NO: 136); LMAN1, Del, A_9, 24, high expression, KKKRNSRRATPTSKGSLRRKYLRV (SEQ ID NO: 137); BPTF, In, A_9, 25, essential, high expression, KKKREETGRRRNDAASDMGKIHISS (SEQ ID NO: 139); RBM26, In, A_9, 25, high expression, KKKTGGIETSAGCKEKETRNFRKAH (SEQ ID NO: 140); TCF7L2, In, A_9, 25, driver, KKKCVRYIQGEGSCLSPPSSDGSLL (SEQ ID NO: 141); GTF3C3, Del, A_9, 27, essential, KKLQKKAPQKRIKLLRMLPALYLMACQ (SEQ ID NO: 142); KMT2C, Del, A_9, 27, driver, KKRNKKTKLWFSLINIHHRKNPLLPMR (SEQ ID NO: 144); DHX36, Del, A_9, 28, essential, high expression, KKCLESGTDHILTEILSISCKKMNQMEL (SEQ ID NO: 145); ZFR, In, A_9, 29, essential, high expression, KKKRSCIESLTKYQQQQQFYSWDSKSATL (SEQ ID NO: 146); SPAG9, Del, A_9, 29, high expression, KKGQAFGSFSADFSAPQVTRLRSLNHLLI (SEQ ID NO: 147); TCF25, In, A_9, 29, high expression, KKKTEKQEKQHGRSIGKRTRRYRSHPRED (SEQ ID NO: 148); TNKS2, Del, A_9, 30, high expression, KKTGMEILLWILLKMEIQIFKICLGEMQLC (SEQ ID NO: 149); CEP57, Del, A_9, 30, essential, KKCKSWKQNSMKKNRKGNACKLRQLSCRLV (SEQ ID NO: 150); SEC63, In, A_9, 32, essential, high expression, KKKETFKKKTYTCAITTVKATETKAGKWSRWE (SEQ ID NO: 151); DYNC1I2, Del, A_9, 36, essential, high expression, KKKKQTRRRKLLLLCKKNQILKKKGEKLKHCFKAWG (SEQ ID NO: 153); SFSWAP, Del, A_9, 37, essential, KKNQESALTMKMMMMKKMGITFIPLSLPPRSVTALKS (SEQ ID NO: 154); FAM60A, In, A_9, 39, high expression, KKKLESCGRCKGWTQSKDYIETKESENSIWEQDKKQPDQ (SEQ ID NO: 155); ACBD3, Del, A_9, 44, high expression, KKGRRKRSEGGVKRKKENVCKRRKRNVGEKKRKGFDGRKRKGDG (SEQ ID NO: 157); PDS5B, In, A_9, 44, high expression, KKKIWTSSTRGGGRRRKTKWKYGTEVQKQTAPSVKESTAESRIS (SEQ ID NO: 158); ASH1L, Del, A_9, 45, high expression, KKQRKVVKNPSQPVPLRNDGITNGNDSTRSCSISLKKSLEKMPLM (SEQ ID NO: 159); CIR1, Del, A_9, 52, high expression, KKYKGRKERKTSVQGITTVILKRRTSLRRESFMKNFLAVTITGKKPRKSPGS (SEQ ID NO: 160); CDC7, Del, A_9, 52, essential, KKTSRILNLQVLKKILRSFMKLYHSLVMCLRLRTKLEKALSALFIWPQHSYK (SEQ ID NO: 161); EIF2B3, Del, A_9, 59, essential, KKKQWSSVTSLEWTAQERGCSSWLMKQTWMKSWSLRDPSYRSILEYVSTRVLWMPTSTV (SEQ ID NO: 162); VCP, Del, A_9, 64, essential, high expression, KKMCSSLALPTGLTSLILPSSDLAVLISSSTSHFLMRSPVLPSSRLTCASPQLPRMWTWSSWLK (SEQ ID NO: 164); ERCC5, Del, A_9, 66, driver, high expression, KKNYGHCNSPLAFLTQLLPRPTSNPWWMTRRDPFCGGNLISTKLENFVSGISAGTERRQMNLCFLY (SEQ ID NO: 165); PRDM2, Del, A_9, 76, high expression, KKKFLIHLRKVDTHHLQVVTKTVTATTADGQRMRRLKCKACRLRWARPEPAAQAPPKSHFPPHPS GPSRTSSLQLR (SEQ ID NO: 166); PDS5B, Del, A_9, 77, high expression, KKNLDLQHQRRRKKKKDKVEIRNRSPKANSTECQGEHSREQNLLNLVQLNPHSPHHRKDEEDHQK RHHHHNQKKMSV (SEQ ID NO: 167); PPP1R10, Del, A_9, 81, essential, high expression, KKKYCHLRLPSQAPLKGKRAQNQAQPNLLPQNQHHLLRQWTQTVQAPRFPLLKSRSSWIQPLWSQ ELWMPSQWRVLEILTN (SEQ ID NO: 168); SLC23A2, In, C_9, 20, high expression, PPPHPRNKQGNFRGRPLLCS (SEQ ID NO: 169); NDUFC2, Del, U_9, 8, essential, high expression, FFLLDIIL (SEQ ID NO: 170); ZC3H11A, In, U_9, 10, high expression, FFFLFHMYQR (SEQ ID NO: 171); MARS, In, U_9, 10, essential, FFFVIWLGAR (SEQ ID NO: 172); NDUFC2, In, U_9, 11, essential, high expression, FFFCWILSCKT (SEQ ID NO: 173); ITSN1, Del, U_9, 11, high expression, FFFNLGYLNLF (SEQ ID NO: 174); COBLL1, Del, U_9, 13, high expression, FFCRCRREYRVTM (SEQ ID NO: 175); RTN3, In, U_9, 13, high expression, FFFNLVFSPILSL (SEQ ID NO: 176); CLOCK, In, U_9, 15, high expression, FFFSNHDRWKHNICV (SEQ ID NO: 177); RYR2, Del, U_9, 16, high expression, FFLPLTFSTLLWDSRH (SEQ ID NO: 178); MS4A6A, Del, U_9, 16, high expression, FFLSSLALYQSPQRKG (SEQ ID NO: 179); ITSN1, In, U_9, 19, high expression, FFFSIWVTSTCFSTDMGTS (SEQ ID NO: 180); UGDH, Del, U_9, 19, high expression, FFLPILMMPSKKLILYLFL (SEQ ID NO: 181); ZMPSTE24, In, U_9, 20, high expression, FFFICCINWSKGAFCCIWFL (SEQ ID NO: 182); ZC3H11A, Del, U_9, 21, high expression, FFSIPHVPKVTAAHSVTVKLQ (SEQ ID NO: 183); MS4A6A, In, U_9, 23, high expression, FFFYHLWLSINRHREKVNQAFGA (SEQ ID NO: 184); COBLL1, In, U_9, 26, high expression, FFFADAEESIGSLCDICSCQECPCCP (SEQ ID NO: 185); ABCC5, Del, U_9, 27, high expression, FFCLRVRCGGWLCGWTSSASPSSPPRG (SEQ ID NO: 186); MARS, Del, U_9, 34, essential, FFCYLAGSKMTSLTSGWNGKRQSCSQLCLLPCTI (SEQ ID NO: 187); UGDH, Del, U_9, 35, high expression, FFLPILMMPSKKLILYLFLCCPTLSFWQREQPSRT (SEQ ID NO: 188); VEZT, Del, U_9, 39, essential, high expression, FFLSAIRKMTYFTSWILDSDSTHYIPSCNRKSCYKRMWS (SEQ ID NO: 189); RYR2, In, U_9, 58, high expression, FFFCRSPSRHCYGIQDIKNHLVLSNSQWQTARINRWLISCCCIPIHCGGIQFFPKILQ (SEQ ID NO: 190); ABCC5, In, U_9, 64, high expression, FFFVYVCDAVAGCAAGPHQHRPHHHHGADDRSYARADSPSLCGSRHLLCCPVNGAVPVYGQTGI (SEQ ID NO: 191); LRPPRC, Del, A_8, 8, essential, high expression, KKGHMIFS (SEQ ID NO: 192); SMC4, In, A_8, 8, essential, high expression, KKIKGSYG (SEQ ID NO: 193); NOP58, Del, A_8, 8, essential, high expression, KKRERTRI (SEQ ID NO: 194); NOL7, In, A_8, 8, essential, high expression, KKTKCQEV (SEQ ID NO: 195); FNBP1, Del, A_8, 8, driver, high expression, KKISLCPF (SEQ ID NO: 196); NCOR1, In, A_8, 8, driver, high expression, KKRRRKER (SEQ ID NO: 197); KRCC1, Del, A_8, 8, high expression, KKAARKLT (SEQ ID NO: 198); SMAP2, In, A_8, 8, high expression, KKIGTCCF (SEQ ID NO: 199); ANKRD12, In, A_8, 8, high expression, KKIKYSRR (SEQ ID NO: 200); CBX5, In, A_8, 8, high expression, KKKERAEQ (SEQ ID NO: 201); ADNP, In, A_8, 8, high expression, KKKGYHAR (SEQ ID NO: 202); RABGAP1, In, A_8, 8, high expression, KKKQFYHW (SEQ ID NO: 203); FEZ2, In, A_8, 8, high expression, KKKRTTVC (SEQ ID NO: 204); PHACTR2, In, A_8, 8, high expression, KKKYQGSW (SEQ ID NO: 205); TM9SF3, In, A_8, 8, high expression, KKKYQSLP (SEQ ID NO: 206); PPP1R1A, In, A_8, 8, high expression, KKNCRMHP (SEQ ID NO: 207); ANK3, In, A_8, 8, high expression, KKNHKSGM (SEQ ID NO: 208); TRDN, In, A_8, 8, high expression, KKRAGKSF (SEQ ID NO: 209); NCOA7, Del, A_8, 8, high expression, KKREKVIS (SEQ ID NO: 210); DLAT, In, A_8, 8, high expression, KKRGGQNQ (SEQ ID NO: 211); NUCB2, In, A_8, 8, high expression, KKRILGAR (SEQ ID NO: 212); ADD3, Del, A_8, 8, high expression, KKRRKLRP (SEQ ID NO: 213); DDX50, Del, A_8, 8, high expression, KNQSEYHL (SEQ ID NO: 214); MED23, In, A_8, 8, essential, KKKCGGGV (SEQ ID NO: 215); TWISTNB, In, A_8, 8, essential, KKKEKKTQ (SEQ ID NO: 216); RTTN, In, A_8, 8, essential, KKKGGWCY (SEQ ID NO: 217); RAE1, In, A_8, 8, essential, KKKLHFPA (SEQ ID NO: 218); HAUS3, In, A_8, 8, essential, KKNWLSQS (SEQ ID NO: 219); KDM5A, In, A_8, 8, driver, KKRIQLAS (SEQ ID NO: 220); CWF19L2, In, A_8, 9, essential, high expression, KKKEQETEI (SEQ ID NO: 221); NOP58, In, A_8, 9, essential, high expression, KKKERERGL (SEQ ID NO: 222); SKIV2L2, In, A_8, 9, essential, high expression, KKSTDCNRY (SEQ ID NO: 223); AKAP9, In, A_8, 9, driver, high expression, KKKEKNVKQ (SEQ ID NO: 224); CCDC88A, Del, A_8, 9, high expression, KKHWIALKI (SEQ ID NO: 225); USP34, In, A_8, 9, high expression, KKIKLYKNL (SEQ ID NO: 226); BBX, In, A_8, 9, high expression, KKIQQPPSI (SEQ ID NO: 227); IQGAP1, Del, A_8, 9, high expression, KKIRNSCLI (SEQ ID NO: 228); ZCCHC11, Del, A_8, 9, high expression, KKISKMIFV (SEQ ID NO: 229); SLTM, Del, A_8, 9, high expression, KKKARILRK (SEQ ID NO: 230); MTDH, In, A_8, 9, high expression, KKKEKEEAR (SEQ ID NO: 231); SYNCRIP, In, A_8, 9, high expression, KKKQRLLLS (SEQ ID NO: 232); PSD3, In, A_8, 9, high expression, KKKVSLRKY (SEQ ID NO: 233); SCN7A, In, A_8, 9, high expression, KKKYFHSIK (SEQ ID NO: 234); NRIP1, In, A_8, 9, high expression, KKRENSLKR (SEQ ID NO: 235); ZNF486, In, A_8, 9, high expression, KKTFEIYRR (SEQ ID NO: 236); DNAJC2, Del, A_8, 9, essential, KKMFLNLVI (SEQ ID NO: 237); WDR55, Del, A_8, 9, essential, KKREDHCGL (SEQ ID NO: 238); LIPT1, Del, A_8, 9, essential, KKSMIEWKI (SEQ ID NO: 239); EIF5B, In, A_8, 10, essential, high expression, KKKSEKQARS (SEQ ID NO: 240); SBNO1, Del, A_8, 10, essential, high expression, KKMEDMIWES (SEQ ID NO: 241); CEP350, Del, A_8, 10, essential, high expression, KNQLLNLIAL (SEQ ID NO: 243); COL12A1, Del, A_8, 10, high expression, KKFHIKVATQ (SEQ ID NO: 244); NUB1, In, A_8, 10, high expression, KKIKLGPEML (SEQ ID NO: 245); ZNF330, In, A_8, 10, high expression, KKKDWCEEEG (SEQ ID NO: 246); NKTR, In, A_8, 10, high expression, KKKNFERESF (SEQ ID NO: 247); SEC31A, In, A_8, 10, high expression, KKNYQETYSR (SEQ ID NO: 248); EPC2, In, A_8, 10, high expression, KKREYGHSCS (SEQ ID NO: 249); RBM6, In, A_8, 10, high expression, KKTHQSRKVK (SEQ ID NO: 250); USP34, Del, A_8, 10, high expression, KNQTLQEFVI (SEQ ID NO: 251); SMAP2, Del, A_8, 10, high expression, KNWNLLFLRR (SEQ ID NO: 252); NUP214, In, A_8, 10, driver, essential, KKSHSLSSVL (SEQ ID NO: 253); BRCA2, Del, A_8, 10, driver, essential, KKYRKTKNQN (SEQ ID NO: 254); POLA1, In, A_8, 10, essential, KKKEVRCSGC (SEQ ID NO: 255); SRPRA, In, A_8, 10, essential, KKKGGQEGRF (SEQ ID NO: 256); RTTN, Del, A_8, 10, essential, KKRRMVLLKS (SEQ ID NO: 257); KDM5A, Del, A_8, 10, driver, KKDPAGKLKK (SEQ ID NO: 259); EIF5B, Del, A_8, 11, essential, high expression, KKIRKTSQVLT (SEQ ID NO: 260); SMC4, In, A_8, 11, essential, high expression, KKKAEIARRKR (SEQ ID NO: 261); RAD50, In, A_8, 11, essential, high expression, KKKYKQQNSTG (SEQ ID NO: 262); NBAS, Del, A_8, 11, essential, high expression, KKSKIKSPFTH (SEQ ID NO: 263); EIF5B, Del, A_8, 11, essential, high expression, KKSRTLMKMIS (SEQ ID NO: 264); PSMD1, Del, A_8, 11, essential, high expression, KNQLTRDWKAS (SEQ ID NO: 265); BIRC6, Del, A_8, 11, essential, high expression, KNWVNTPRRRL (SEQ ID NO: 266); JAK1, In, A_8, 11, driver, high expression, KKKDSRCNPSP (SEQ ID NO: 267); RPL22, In, A_8, 11, driver, high expression, KKKEASSEVHS (SEQ ID NO: 268); ANKRD12, In, A_8, 11, high expression, KKIKIGKKHQR (SEQ ID NO: 269); ASH1L, In, A_8, 11, high expression, KKKKRKKTSVD (SEQ ID NO: 270); ARHGAPS, In, A_8, 11, high expression, KKKYDRKFLFV (SEQ ID NO: 271); WDR60, In, A_8, 11, high expression, KKKYQAGICSV (SEQ ID NO: 272); GOLGA4, Del, A_8, 11, high expression, KKLRLAVLVNN (SEQ ID NO: 273); DLAT, Del, A_8, 11, high expression, KKRGTKSMKVT (SEQ ID NO: 274); KIAA1109, Del, A_8, 11, high expression, KNSLITQMMKH (SEQ ID NO: 276); STIL, Del, A_8, 11, driver, essential, KKIRHAFYLVL (SEQ ID NO: 277); TTF1, In, A_8, 11, essential, KKKEKVQSPGI (SEQ ID NO: 278); ZMYND8, In, A_8, 12, essential, high expression, KKKAQAYKPSGD (SEQ ID NO: 279); NIPBL, Del, A_8, 12, essential, high expression, KKSLILSFQRVK (SEQ ID NO: 280); COPA, Del, A_8, 12, essential, high expression, KKTCPLVRWNRM (SEQ ID NO: 281); FBXO21, Del, A_8, 12, high expression, KKFFTTCGNRRS (SEQ ID NO: 282); ZCCHC11, Del, A_8, 12, high expression, KKFQAPLLLWTK (SEQ ID NO: 283); FAM98A, In, A_8, 12, high expression, KKIKGNISKSST (SEQ ID NO: 284); EAPP, In, A_8, 12, high expression, KKKEETTQDSNK (SEQ ID NO: 285); TTC3, In, A_8, 12, high expression, KKKQISRSSQSL (SEQ ID NO: 286); ROCK1, In, A_8, 12, high expression, KKMGNSFSKENP (SEQ ID NO: 287); PSME4, In, A_8, 12, high expression, KKNLIQFPKDSP (SEQ ID NO: 288); NUCB2, Del, A_8, 12, high expression, KKNSWSQIAGRH (SEQ ID NO: 289); ADNP, Del, A_8, 12, high expression, KKRLPCKVTESS (SEQ ID NO: 290); AP3B1, In, A_8, 12, high expression, KKTSYRHENACF (SEQ ID NO: 291); BZW1, Del, A_8, 12, high expression, KKTTSQSQLSSE (SEQ ID NO: 292); BRCA2, In, A_8, 12, driver, essential, KKNSVSTTTGFR (SEQ ID NO: 293); BRCA2, In, A_8, 12, driver, essential, KKNTERPKIRTN (SEQ ID NO: 294); VPS54, In, A_8, 12, essential, KKIRSHRRKETS (SEQ ID NO: 295); TTF1, In, A_8, 12, essential, KKKEEKVQSPGI (SEQ ID NO: 296); TAF1B, In, A_8, 12, essential, KKKPFKFSVQLD (SEQ ID NO: 297); HAUS1, In, A_8, 12, essential, KKNIWRSSYSTV (SEQ ID NO: 298); TOPBP1, In, A_8, 12, essential, KKNPAKTRIWHC (SEQ ID NO: 299); THOC1, Del, A_8, 12, essential, KNVPLTKLSEVF (SEQ ID NO: 300); EIF5B, In, A_8, 13, essential, high expression, KKKERTEGQKTEF (SEQ ID NO: 301); INTS8, Del, A_8, 13, essential, high expression, KKSFSKQWQNFTF (SEQ ID NO: 302); AKAP13, In, A_8, 13, high expression, KKKEEQNQPLSAR (SEQ ID NO: 303); TMPO, Del, A_8, 13, high expression, KKLINPDKKIKMI (SEQ ID NO: 304); HSPH1, Del, A_8, 13, high expression, KKLTSLQKLKSPK (SEQ ID NO: 305); BAZ1A, Del, A_8, 13, high expression, KKQDRIFRVFQNH (SEQ ID NO: 306); RERE, Del, A_8, 13, high expression, KKSLVMKGQIPVR (SEQ ID NO: 307); RAE1, Del, A_8, 13, essential, KKITFSCVMQPKS (SEQ ID NO: 309); CEP57, In, A_8, 13, essential, KKKEVKTTRKEKF (SEQ ID NO: 310); MIS18BP1, In, A_8, 13, essential, KKKSGSSATGMDD (SEQ ID NO: 311); MED23, Del, A_8, 13, essential, KKMWRRSIGSGSQ (SEQ ID NO: 312); REV1, In, A_8, 13, essential, KKNENRQVCTCCN (SEQ ID NO: 313); KNTC1, In, A_8, 13, essential, KKTEAKAPEVNTS (SEQ ID NO: 314); HAUS1, Del, A_8, 13, essential, KKYLEIILFHSMR (SEQ ID NO: 315); WRN, In, A_8, 13, driver, KKIGNNCTAAEMS (SEQ ID NO: 316); DDX27, In, A_8, 14, essential, high expression, KKKGGDDSRGKVSV (SEQ ID NO: 317); SSB, Del, A_8, 14, essential, high expression, KKMKKENKIKWKLN (SEQ ID NO: 319); UBRS, In, A_8, 14, driver, high expression, KKRRGRTARAARRN (SEQ ID NO: 320); DDX50, In, A_8, 14, high expression, KKIKASIIFRYFYS (SEQ ID NO: 321); SEC31A, Del, A_8, 14, high expression, KKLPRNLFQMSTSF (SEQ ID NO: 323); ZCRB1, In, A_8, 14, high expression, KKMETQFRSPLNIR (SEQ ID NO: 324); FBXO21, In, A_8, 14, high expression, KKNSLLPAATEDLK (SEQ ID NO: 325); ZCRB1, Del, A_8, 14, high expression, KKRKLLNQKKKLRK (SEQ ID NO: 326); COIL, Del, A_8, 14, high expression, KKRLRIPSLRKYRQ (SEQ ID NO: 327); PHF20L1, In, A_8, 14, high expression, KKSEIGRQKLNSIW (SEQ ID NO: 328); TRDN, Del, A_8, 14, high expression, KKSREKLLKPNKGL (SEQ ID NO: 329); CTSC, In, A_8, 14, high expression, KKSSGVPSEAGYSI (SEQ ID NO: 330); BRE, Del, A_8, 14, high expression, KKTTGLVNFQLVSF (SEQ ID NO: 331); REV3L, In, A_8, 14, essential, KKISEETSGNSQTE (SEQ ID NO: 332); NUFIP1, In, A_8, 14, essential, KKKEKKGTSFSLFL (SEQ ID NO: 333); CEP57, In, A_8, 14, essential, KKKEVKTTRKVHKP (SEQ ID NO: 334); UBE4B, Del, A_8, 15, essential, high expression, KKHQRCAASQQSASF (SEQ ID NO: 335); ZMYND8, Del, A_8, 15, essential, high expression, KKSPSLQTQWRLKRS (SEQ ID NO: 336); PAFAH1B1, Del, A_8, 15, essential, high expression, KNGHLLLDYKRRLWN (SEQ ID NO: 337); SMC4, Del, A_8, 15, essential, high expression, KNLAGFQEYMEDWGT (SEQ ID NO: 338); QKI, Del, A_8, 15, driver, high expression, KKRRSKIEASPIGSI (SEQ ID NO: 339); FXR1, Del, A_8, 15, high expression, KKIPSLNAQWMFLRI (SEQ ID NO: 340); ZCCHC11, In, A_8, 15, high expression, KKNSKLLYFCGQSEY (SEQ ID NO: 342); TTC3, In, A_8, 15, high expression, KKRNPRKTKITEEEN (SEQ ID NO: 343); R3HDM2, In, A_8, 15, high expression, KKTGGRICKQKQVYI (SEQ ID NO: 344); ITGA6, Del, A_8, 15, high expression, KNSGSQSGTKMKATH (SEQ ID NO: 345); NUCB2, Del, A_8, 15, high expression, KNYNKEFLHQGQLEN (SEQ ID NO: 346); WRN, Del, A_8, 15, driver, KNWKQLHSSGNVLNG (SEQ ID NO: 347); TUBGCP2, In, A_8, 16, essential, high expression, KKKFRPAPPHLPSMGV (SEQ ID NO: 348); DIDO1, In, A_8, 16, essential, high expression, KKRDHSEEGSGGPCAE (SEQ ID NO: 349); NOL7, Del, A_8, 16, essential, high expression, KNKMPRGLKDGGWSER (SEQ ID NO: 350); JAK1, Del, A_8, 16, driver, high expression, KNQQLKWTPHILKSAS (SEQ ID NO: 351); LRRK2, Del, A_8, 16, high expression, KKGNFQRTTCHSILSS (SEQ ID NO: 352); CSPP1, In, A_8, 16, high expression, KKKCNYGIIRNEKTAS (SEQ ID NO: 353); MPP6, In, A_8, 16, high expression, KKKEKDDVSHNQKCRI (SEQ ID NO: 354); ZMYM2, In, A_8, 16, high expression, KKKGSQEKGCIRIPVS (SEQ ID NO: 355); RHOQ, In, A_8, 16, high expression, KKKNRIKMYKLLFNYV (SEQ ID NO: 356); OSBPL1A, Del, A_8, 16, high expression, KKMIRKIQKRRRTANR (SEQ ID NO: 357); SUCLG2, In, A_8, 16, high expression, KKRKLYAHKTRNRKFK (SEQ ID NO: 358); FGF7, Del, A_8, 16, high expression, KKRRKNKKQPTFFLWQ (SEQ ID NO: 359); CCDC66, In, A_8, 16, high expression, KKSKEADGIASFGRKK (SEQ ID NO: 360); FMR1, In, A_8, 16, high expression, KKTFRYKGKQHPFFST (SEQ ID NO: 361); RABGAP1, Del, A_8, 16, high expression, KKTVLSLVTISRFVLS (SEQ ID NO: 362); COG2, In, A_8, 16, essential, KKKDVCIEAYTSYSVS (SEQ ID NO: 363); TCOF1, In, A_8, 16, essential, KKRKEEESKKGLNQRF (SEQ ID NO: 364); TWISTNB, Del, A_8, 16, essential, KKRKENTVKRPNLPHL (SEQ ID NO: 365); BOD1L1, In, A_8, 16, essential, KKSSLKQKEKKRFKEC (SEQ ID NO: 366); SPECC1, In, A_8, 16, driver, KKKHTWKCITDIRLLK (SEQ ID NO: 367); SLC3A2, In, A_8, 17, essential, high expression, KKKEHPCHSGPYSQLPG (SEQ ID NO: 368); SF3B2, In, A_8, 17, essential, high expression, KKTESSAPGQPWGQQEI (SEQ ID NO: 369); SWAP70, In, A_8, 17, high expression, KKKPHKESPAHYRRRCI (SEQ ID NO: 370); FILIP1L, In, A_8, 17, high expression, KKTKSTRKQEQRFRKRD (SEQ ID NO: 371); STIL, In, A_8, 17, driver, essential, KKKFVMLFTWFSDSRRR (SEQ ID NO: 372); POLA1, In, A_8, 17, essential, KKIKEGAPRSPRKTEKG (SEQ ID NO: 373); REV3L, Del, A_8, 17, essential, KNFRRDFRKFSNRMISL (SEQ ID NO: 374); TCERG1, Del, A_8, 18, essential, high expression, KKRESTLGNFWMKLLQLP (SEQ ID NO: 375); AKAP9, Del, A_8, 18, driver, high expression, KKREKRQAVNMMCQHTMI (SEQ ID NO: 376); NUCB2, In, A_8, 18, high expression, KKITTRNSSIRASWRIEV (SEQ ID NO: 377); GPATCH4, In, A_8, 18, high expression, KKKEKEKEAERGGRSYSI (SEQ ID NO: 378); PLEKHA5, Del, A_8, 18, high expression, KKLKTFHMKCFLNLSQME (SEQ ID NO: 379); ZNF43, Del, A_8, 18, high expression, KNFSNAKNVANHFACFHI (SEQ ID NO: 380); RAB14, Del, A_8, 18, high expression, KNLWLIVLTQLVLNLVQE (SEQ ID NO: 381); NAA15, In, A_8, 18, essential, KKKCRKRKAAEKSEKEEG (SEQ ID NO: 382); WDR55, In, A_8, 18, essential, KKKGRTTAGSEQQDLEHR (SEQ ID NO: 383); CEP57, Del, A_8, 18, essential, KKRSQNHQKSPQALAMPW (SEQ ID NO: 385); SMC4, In, A_8, 19, essential, high expression, KKIWQDSRNIWKIGGLRSH (SEQ ID NO: 386); USP1, In, A_8, 19, essential, high expression, KKKCRSYWTSWRTKEQSRL (SEQ ID NO: 387); EXOC1, Del, A_8, 19, essential, high expression, KKKPNKNTQITFSLMSFTL (SEQ ID NO: 388); COPA, In, A_8, 19, essential, high expression, KKKPVPWCGGIGCERNNWG (SEQ ID NO: 389); RIF1, Del, A_8, 19, essential, high expression, KKRQTLRTLSLRGMVPRTL (SEQ ID NO: 390); ESF1, Del, A_8, 19, essential, high expression, KKTLFNILQTLLSKKNKGH (SEQ ID NO: 391); CUL5, Del, A_8, 19, high expression, KKIIVVENYIGIISCQMEL (SEQ ID NO: 392); FGF7, In, A_8, 19, high expression, KKNEERTKNSPLSSYGNNL (SEQ ID NO: 393); ZNF330, Del, A_8, 19, high expression, KKRLVRGRRLRTAENVKNN (SEQ ID NO: 394); MAP1A, In, A_8, 19, high expression, KKRQGEKRDQKGEERAQEG (SEQ ID NO: 395); DST, In, A_8, 19, high expression, KKRSCNFLSRTVGCFSPSC (SEQ ID NO: 396); TTC3, In, A_8, 19, high expression, KKRSKKVSTRKNGGGLKRK (SEQ ID NO: 397); CHD2, In, A_8, 19, high expression, KKSKSQKTCPQKNSAQTSC (SEQ ID NO: 398); TUBGCP2, Del, A_8, 20, essential, high expression, KKIQASTSPSSQHGCMRDLP (SEQ ID NO: 399); CYP51A1, In, A_8, 20, high expression, KKMLFRTENSLWRESASFNL (SEQ ID NO: 401); MTDH, Del, A_8, 20, high expression, KKRKRRSKVKITLLHRTQKN (SEQ ID NO: 402); ZCRB1, Del, A_8, 20, high expression, KNGNPVQESPQHQMIQDAQG (SEQ ID NO: 403); SECISBP2L, Del, A_8, 20, high expression, KNYRKLYQKQLEKRIKHLCS (SEQ ID NO: 404); BRCA1, In, A_8, 20, driver, essential, KKKVQPNASQAQQKPTTHGR (SEQ ID NO: 405); CAMTA1, In, A_8, 20, driver, KKIPAEPTGCCAHPKVLPKL (SEQ ID NO: 407); CUL2, Del, A_8, 21, essential, high expression, KKMTWQICTSYSVLCPLVYLI (SEQ ID NO: 408); SON, Del, A_8, 21, essential, high expression, KNIKDSQKNLSQRRNLMMMGT (SEQ ID NO: 409); TFDP2, Del, A_8, 21, high expression, KKKSSGLACLPILLRNVRIWR (SEQ ID NO: 410); HIBCH, In, A_8, 21, high expression, KKRLHGSHNTKQTKVPQCTDS (SEQ ID NO: 411); ZC3H11A, Del, A_8, 21, high expression, KNIGSRKQRDKKAKRIQLASS (SEQ ID NO: 412); CEP152, Del, A_8, 21, essential, KKLNVTCFVIFRRVRNELQKW (SEQ ID NO: 413); C11orf57, Del, A_8, 21, essential, KKSRKKGHTKNRRKAKRKPQI (SEQ ID NO: 414); SF3B2, Del, A_8, 22, essential, high expression, KNGKLSPRTAVGAARNIRSSSF (SEQ ID NO: 415); AKAP7, Del, A_8, 22, high expression, KKREKIINPTISCPFQSPTKRL (SEQ ID NO: 416); ZNF43, In, A_8, 22, high expression, KKTFQMQRMWQIILHASTSSST (SEQ ID NO: 417); COPB2, Del, A_8, 23, essential, high expression, KNHLNQILEQKVSTAASYWESDL (SEQ ID NO: 418); SP100, In, A_8, 23, high expression, KKKEVQGSQCTQEASFGLFPVLL (SEQ ID NO: 419); ARFGAP3, Del, A_8, 23, high expression, KKMLTQTDSAWDLEIAEVLFHIQ (SEQ ID NO: 420); TMOD3, In, A_8, 23, high expression, KKRENIYPQTETCTDFYRRKSVS (SEQ ID NO: 421); SYNCRIP, Del, A_8, 23, high expression, KKTEAFAFLNMKITKQLPRQGVG (SEQ ID NO: 422); ZNF638, Del, A_8, 23, high expression, KKVTWMKRRRRNLILRKPEWIFK (SEQ ID NO: 423); FNIP1, Del, A_8, 23, high expression, KNLQIRLCLLHFLVRLPRQRLLS (SEQ ID NO: 424); NAA15, Del, A_8, 23, essential, KKMQKKKSSREIRKRRRMMMMRR (SEQ ID NO: 425); PPP1R12A, Del, A_8, 24, essential, high expression, KKIAQLMKDQRIIKVAPLVEDKMI (SEQ ID NO: 426); EXOC1, In, A_8, 24, essential, high expression, KKRSQTKIHRSPSVLCHLLFRTTS (SEQ ID NO: 427); CEP350, Del, A_8, 24, essential, high expression, KNIYETKERMIKCPRSGRNSVVCF (SEQ ID NO: 428); ASH1L, Del, A_8, 24, high expression, KKEKEENLGGLKWWQEAHAGLQKG (SEQ ID NO: 429); WDR60, Del, A_8, 24, high expression, KKIPSRHMFSVTKIMLKETFKRRK (SEQ ID NO: 430); P4HB, In, A_8, 24, high expression, KKKRLCGVLCPMVWSLQTVGSHLG (SEQ ID NO: 431); ZMYM2, In, A_8, 24, high expression, KKKSFNDGKQSVSSIPSVFLVWNR (SEQ ID NO: 432); AHI1, Del, A_8, 24, high expression, KKQKRRLKQLQIIMKMLMVMVFMK (SEQ ID NO: 433); ZC3H11A, In, A_8, 24, high expression, KKTSAAGSRETKKQKGYNLHQAKD (SEQ ID NO: 434); NSRP1, Del, A_8, 24, essential, KKRRKIIPNCFWGKTESPSIFTTC (SEQ ID NO: 435); TET1, Del, A_8, 24, driver, KKRKTANYERQPREPTKMWHQSRL (SEQ ID NO: 436); ESF1, In, A_8, 25, essential, high expression, KKKHCSTYYRLFSRRKTKDIRLRHL (SEQ ID NO: 437); CPVL, Del, A_8, 25, high expression, KKFGRSLNLTVKWLVTSGKRVTSIR (SEQ ID NO: 438); VPS13C, Del, A_8, 25, high expression, KKLVCSALISLTLKGNLFTLLTLLM (SEQ ID NO: 440); ARID4A, In, A_8, 25, high expression, KKRLRRINGRGSQIRSRNAFNRSEE (SEQ ID NO: 441); BCL10, Del, A_8, 25, driver, KKYSVEKTLKKFLVEHQVEKGLENC (SEQ ID NO: 442); SBNO1, In, A_8, 26, essential, high expression, KKKEEKYRSRFYSKCLISIRSWIKTT (SEQ ID NO: 443); TMOD3, Del, A_8, 26, high expression, KKGKYLSPNRNLYRLLQKKKCLLIQN (SEQ ID NO: 445); AKAP7, In, A_8, 26, high expression, KKKEKRLSTQLFPVHSNHQQRDYKRN (SEQ ID NO: 446); VEGFB, In, A_8, 26, high expression, KKKGQCCEARQPQAPLPTLHPAPPAP (SEQ ID NO: 447); LARP1B, Del, A_8, 26, high expression, KNSGEKFFRISKKKPKKTTNLVSCMD (SEQ ID NO: 448); SLC39A10, Del, A_8, 26, high expression, KNTILKNFLSVMVKMEDYPFLVWRNF (SEQ ID NO: 449); YY1, Del, A_8, 26, essential, KKILTMRQWLKNRSLERTHLLIIQNI (SEQ ID NO: 450); NAA35, In, A_8, 26, essential, KKKESSPIEPRDHNEPSISEHVCWNV (SEQ ID NO: 451); POLA1, Del, A_8, 26, essential, KKRSTLLWLLSQRRMGIMSPNRSSKD (SEQ ID NO: 452); NCOR1, Del, A_8, 27, driver, high expression, KKKKKRKMKRKKMKKKTPKKIPRKRTR (SEQ ID NO: 453); JAK1, Del, A_8, 27, driver, high expression, KKRFQMQPLSLMPAHWSICLLRDSMIW (SEQ ID NO: 454); PRDM2, In, A_8, 27, high expression, KKKSASTESNSSEKQICKAEGRLEKCL (SEQ ID NO: 455); HERC1, Del, A_8, 27, high expression, KKLTPVEKLRRKISELLLGNNMQPNSI (SEQ ID NO: 456); LARP7, In, A_8, 27, high expression, KKNNIPNKIRVRNGNRQWSTSKHWNEK (SEQ ID NO: 457); TAF1D, Del, A_8, 27, high expression, KKRKRGGTSQQEDHGEDQKEGEILYTH (SEQ ID NO: 458); RBM6, Del, A_8, 27, high expression, KNPPVKESQVARRKCLKEMARRKKTEE (SEQ ID NO: 459); C11orf57, In, A_8, 28, essential, KKKAEKKVTQKTEEKQKGSHRYNSRFLE (SEQ ID NO: 460); HAUS3, Del, A_8, 28, essential, KKLVIPKLIILMEKTLTGCLRALKMNRF (SEQ ID NO: 461); RPL9, Del, A_8, 29, essential, high expression, KKRGSGLTNGGVTERNWLPFGLFVVMYRT (SEQ ID NO: 462); UBRS, Del, A_8, 29, driver, high expression, KKKGKNSPCCQKKLRVQNQGHLLMILLHN (SEQ ID NO: 463); RTN3, Del, A_8, 29, high expression, KKGRISTWKPEMQQLLKHHLIVITSLLVL (SEQ ID NO: 464); ZNF43, Del, A_8, 29, high expression, KKIILDTNSTNVKNVAKLLTSPQSLLPIR (SEQ ID NO: 465); CCDC149, In, A_8, 29, high expression, KKIKNSGSFWFVESSRGQGHHTVQRPHSS (SEQ ID NO: 466); PHF20L1, Del, A_8, 29, high expression, KKRKRNLSNMTIQTMKTVPSNFWKGALLH (SEQ ID NO: 467); AKAP13, Del, A_8, 29, high expression, KKRRTKPAALSPVMVPRQKYQQRVKRSSA (SEQ ID NO: 468); TAF1B, Del, A_8, 29, essential, KKALQVFSSTGLKRTLIERVSMDTAFRES (SEQ ID NO: 469); MAPKAP1, In, A_8, 29, essential, KKISQREASNFWEAVDIICTPRTVPSAAE (SEQ ID NO: 470); TAF1D, In, A_8, 30, high expression, KKKEKEEVPANRKTTGKTRRKEKSYILTNR (SEQ ID NO: 471); SNX25, In, A_8, 30, high expression, KKKFFFIILIFGKTSSRLLLPPGGGDRGGQ (SEQ ID NO: 472); RTN3, In, A_8, 30, high expression, KKRQNKYMETRNATVTKTPFNSYNVVTCTM (SEQ ID NO: 473); BRCA2, Del, A_8, 30, driver, essential, KKLSINNYRFQMKFYFRFTSHGSPFTSANF (SEQ ID NO: 474); NUP214, Del, A_8, 30, driver, essential, KKSFLVLRFMSQIILSEFWMCCGLVPTSSP (SEQ ID NO: 475); MDN1, In, A_8, 30, essential, KKKGHCCIQGCVWFKFQPIHGNQTISYHSL (SEQ ID NO: 476); VPS54, Del, A_8, 30, essential, KNQEPQKKGNQLKFLLSRDNSMQLLEPYCC (SEQ ID NO: 477); NKTR, Del, A_8, 31, high expression, KKIAFHIKSIAAALKRHFTVNMSKVETGLHV (SEQ ID NO: 479); BAZ1A, In, A_8, 31, high expression, KKSKTESSEFSRTTNYSSFILDQPYPSFALT (SEQ ID NO: 480); SLC3A2, Del, A_8, 32, essential, high expression, KKRASVSFWTLLPTTGVRTRGSPLRLTLWPPR (SEQ ID NO: 481); CBX5, Del, A_8, 32, high expression, KKRESRAMISLGALREDWNQKRSLGQQIPVVI (SEQ ID NO: 482); SWAP70, Del, A_8, 32, high expression, KKTSQRIPCSLQKKMHLKYGLFSTFYLRTSIH (SEQ ID NO: 483); TM9SF3, Del, A_8, 32, high expression, KKVSVITMKLWEKHFKGLNWNLVVWILNLKMM (SEQ ID NO: 484); VEGFA, Del, A_8, 32, high expression, KNQFEERERGKNESARNPGISPGACTLVPAAV (SEQ ID NO: 485); LIAS, Del, A_8, 32, essential, KKRNSYRMDQTFKILYLVILQTGAPGMNIKET (SEQ ID NO: 486); CEP57, Del, A_8, 32, essential, KKRSQNHQKRKVLGTILVHNHIIDYAWVICHL (SEQ ID NO: 487); FBXO38, Del, A_8, 33, high expression, KKTRMFIPAAAAPPPAQWETPAHTTLLLKAPTL (SEQ ID NO: 488); TAF1, In, A_8, 33, essential, KKKGQDERTREASFRWWRDVFYAHTSGPHRQRW (SEQ ID NO: 489); CAMTA1, Del, A_8, 33, driver, KNSSRADGLLCSSKSTTEVIRNVAKDGRLAGRL (SEQ ID NO: 490); ATP8B1, Del, A_8, 34, high expression, KKMILFQLTFSCCLALSLTASAMWKQQNWMEKPI (SEQ ID NO: 491); ZMYM2, Del, A_8, 34, high expression, KKREPRERLYQDTSLMMIVLTIQNAAFLSNIRMA (SEQ ID NO: 492); SRPRA, Del, A_8, 34, essential, KKRGPRRKVLMVLWLPANQSLQKSQVFQWVLRTE (SEQ ID NO: 493); TOPORS, Del, A_8, 35, high expression, KKIITVRGSITTMKGTDQGACLVTDQGLHLPGLTG (SEQ ID NO: 494); LNPEP, In, A_8, 35, high expression, KKKPVNWTNSAICNKWEIVSMGTDQASHCRCATTL (SEQ ID NO: 495); VDAC1, Del, A_8, 35, high expression, KKMLKSRQGTSGSTLTWAATWISTLLGLPSGVLWC (SEQ ID NO: 496); TCOF1, Del, A_8, 35, essential, KKKRRRKQKRPQPKILSHRPRRKRRKRRRQQSRLY (SEQ ID NO: 497); PMS2, Del, A_8, 35, driver, KKTCPFPDCERPFLFVTQQRTSLTAQRLQNQEGAL (SEQ ID NO: 498); PPP1R12A, Del, A_8, 36, essential, high expression, KKIAQLMKDQRIIKVPQIVCGLRIVLRKKRTVFLRQ (SEQ ID NO: 499); SLTM, Del, A_8, 36, high expression, KKIRVIEVARHKPLSKKKRKDRLRNLKKKKARILRK (SEQ ID NO: 500); EIF3J, In, A_8, 37, essential, high expression, KKRGSRSKTRGKNFRKEKNSREDKRERTATEEKARRN (SEQ ID NO: 501); VEGFB, In, A_8, 37, high expression, KKKGQCCEARQGCHSPPPSPAPFCSGLGLCPRSTLPS (SEQ ID NO: 502); PPP1R1A, Del, A_8, 38, high expression, KKLQNASLKLTREAVRNPAQKNPQPIYHHWIPREPTRS (SEQ ID NO: 503); VEGFA, In, A_8, 39, high expression, KKISSRKGKGAKTKAQEIPSLWALLRAEKAFVCTRSADV (SEQ ID NO: 505); NUFIP1, Del, A_8, 39, essential, KKRKERNQFFTFFVIPVIVVLKIKKSMTNTCLNIQNALN (SEQ ID NO: 506); TET1, In, A_8, 39, driver, KKKEKQPTTKDNQGSQQKCGISQDFKPWKIKAINSRKRC (SEQ ID NO: 507); CCDC88A, Del, A_8, 41, high expression, KKIMKLSNRDKMRKGWYRALLQYLVKTTNGSEKVKKRLENF (SEQ ID NO: 509); PSME4, Del, A_8, 41, high expression, KKPYPISEGLTMTTGRNINSNSLMTNCLFSPIFLCHHAIMH (SEQ ID NO: 510); CWF19L2, Del, A_8, 42, essential, high expression, KKRARNRNMKKTMSHLHHQALKMSGLRLFHPRLLTRKKPGK (SEQ ID NO: 512); TCERG1, Del, A_8, 42, essential, high expression, KKSLRKILDVLSSPPVTGKNKENLKNISETNISQPKLTSGRF (SEQ ID NO: 513); PHF20L1, Del, A_8, 43, high expression, KKMKLTLAVLPTLRNLHCYPQLCLQGRLAARNANMNLEILLGV (SEQ ID NO: 514); EML4, Del, A_8, 44, driver, KKRNLILMIKVHKFEHHLLPSPLHNLSKYTDKLQKARMLLPPKA (SEQ ID NO: 515); FBXO38, In, A_8, 45, high expression, KKKQGCLSQLQQHHRQHSGKLQLTQHCFSKPRLCKDGEQRRLFRA (SEQ ID NO: 516); ANKRD12, Del, A_8, 45, high expression, KNQNWKKTSKMINQPRKSMCQKRGTLKRNETRLKRKARNLLGRKK (SEQ ID NO: 517); PMS2, In, A_8, 45, driver, KKRRVHFQTARGLFSSSHNREQASQPKDSRTKKEPSRTEKGYAVF (SEQ ID NO: 518); PTPN13, Del, A_8, 45, driver, KNPSLALMCFLRRRSGLHPWTCFVQLTETSLQERLPHIVVVTLRQ (SEQ ID NO: 519); TTF1, Del, A_8, 46, essential, KKRKSPVTGNMRHWPCLKDRKQAERPGLICRNPSLLWAWMMKLHNY (SEQ ID NO: 521); PHACTR2, Del, A_8, 48, high expression, KKQLALKHQLRHPLHPPHLVPKLQRRQFLAKQGQWGPPRAREKLTSSQ (SEQ ID NO: 522); BRCA1, Del, A_8, 48, driver, essential, KKSTTKCQSGTAETYNSWKVKNLQLEPRRVTSQMNRQVKDMTAILSQS (SEQ ID NO: 524); MAP1A, Del, A_8, 49, high expression, KKTRRKKRSKRRGKSSRRMKEGRRRRRMPRRRRRGKIPNLSSRRFSPQT (SEQ ID NO: 525); RNPC3, In, A_8, 50, essential, KKRTWLFNSRKWNCTKPWADFSFKFMPQVYVPTTFQHNPSKHCKCLGKRA (SEQ ID NO: 526); SNX25, Del, A_8, 52, high expression, KKILFHYPHFWKDFLATSSPTRRRRQRRTVTCQIMVMMWMGGKTPWLNHVSC (SEQ ID NO: 527); UBE4B, In, A_8, 53, essential, high expression, KKSTKDVQPASSQPASEQHPLTVHIPYCFSTTRLPNTAQVLAAAVLPSAVYAV (SEQ ID NO: 528); BZW1, In, A_8, 55, high expression, KKKQHPRASCHRNSLVKCNEHCGMEQKRGACSRASHQALEAIQPSTCCLYYSRSV (SEQ ID NO: 529); EAPP, Del, A_8, 56, high expression, KKRRNNTRFQQMTNYCMILKKITEIRPGLMHREGVTMVWDHRDHVNNSLFQIVMLS (SEQ ID NO: 530); VEGFA, Del, A_8, 56, high expression, KNQFEERERGKNESARNPVPVGLAQSGESICLYKIRRRVNVPAKTQTRVARRGSLS (SEQ ID NO: 531); OSBPL1A, Del, A_8, 58, high expression, KNDLRKNKEQPAKTGPSQKRTGRRGGSIKVLIPTMEHRTGFTLAATGTEITSICLTFI (SEQ ID NO: 533); PRPF4B, Del, A_8, 59, essential, high expression, KKRSINTEVNIRNINIPQKKTRIKNINISINIRNTKEKRLLMLLIKRVCLQQKELNLMI (SEQ ID NO: 534); CD3EAP, In, A_8, 59, essential, KKKSAAERTRGSRACGDRAHSGDTGASGSAVPVHHQEEEEAQRERNLRARRQDSEAGTD (SEQ ID NO: 535); DIS3, Del, A_8, 60, essential, KKFMRNFLNMLCFENILLHLHQIMKFLLRQPGQGIWKLRLIQPSLWLSLWIRPNLLLFHI (SEQ ID NO: 536); VEGFA, Del, A_8, 62, high expression, KNQFEERERGKNESARNPGISPGAFPVGLAQSGESICLYKIRRRVNVPAKTQTRVARRGSLS (SEQ ID NO: 537); TOPBP1, Del, A_8, 62, essential, KKSSKDKNLALLREHLVGGRLFYMWISLEKQASNAFFSQEEQRCYLVILYLYLKRPHIFFLT (SEQ ID NO: 538); SPECC1, Del, A_8, 64, driver, KKTYMEMHYGHQAPQVAMLPKLLCRQMLPTLSTLQQRHPQGPCPPPVTPLRVQSVLLLGVPQTA (SEQ ID NO: 539); SORBS2, Del, A_8, 66, high expression, KKIRLLRNRLKRQNVKHLCLRLWTLPLKTSVTKLKLKRKGGACRTTASCTASSVSCCQMFPRGTHP (SEQ ID NO: 540); MDN1, Del, A_8, 66, essential, KKRSLLYSRMCLVQIPTHTWEPDYFVSLPMMFSWATQSFPVGAVFLTRPAIPCCSCTSHSNPWSQS (SEQ ID NO: 541); GPATCH4, Del, A_8, 74, high expression, KKRKRKGGRKRRKKLQHLKGMMQMRSTQNMLSRTSEKARRRKGDIKKERSQMKERVQLKGMR RRTLQEQVGLGN (SEQ ID NO: 542); PSD3, Del, A_8, 74, high expression, KKSLPQKVLRRKLTEHIQRPSVVLEVLLTHFWTFLMIQMLLCTKVDSWLGKFMQIWMERRLQEEN EDGKPFMLY (SEQ ID NO: 543); SRFBP1, Del, A_8, 76, essential, KKTKDLHHSLVTVMAEKNFVKRRRNILMIAQKKGFTSSLPCLKIVIAVTTSSLGKSDGHERRKVVV ILQLRNKNH (SEQ ID NO: 544); PHACTR2, Del, A_8, 81, high expression, KKIPRLLVSRPPSLHPSQQAETRPERLLAPLIQKKQLALKHQLRHPLHPPHLVPKLQRRQFLAKQGQ WGPPRAREKLTSSQ (SEQ ID NO: 545); EIF5B, Del, A_8, 84, essential, high expression, KKRKDRRAKNRVLMIMIAKNWKIKIQNQKRLQNRKWKCTLGVMMMMILTNFLKKLKGKLKNQI RSGMGQRRMRITVKKLKSVQE (SEQ ID NO: 546); TTF1, Del, A_8, 88, essential, KKRKSPITRNLRQWPCLRASRVHTLKDHRWAVRLGLWKAVQLLKGSRNPTVQRRSLRKGSLRLSK GHECLVMIFQCPVRTLRAHSLIQ (SEQ ID NO: 547); RAPGEF2, Del, A_8, 99, high expression, KKFFLCLKKEVWNVTRNRLKIQYQMHLRSFLLLLLLHRVLQGKAILWLPVVLWIIFQILVTVKFLH DPVLLAIRLLTQCQSHCTMRGARGILSASWKQT (SEQ ID NO: 548); MYH11, Del, C_8, 8, driver, high expression, PPHRKLRS (SEQ ID NO: 549); MYH11, In, C_8, 9, driver, high expression, PPPTGNFAV (SEQ ID NO: 550); BCL9, In, C_8, 9, driver, PPPSIPDDP (SEQ ID NO: 551); LRP1, In, C_8, 11, high expression, PPPRCSPAWNL (SEQ ID NO: 552); SAFB2, Del, C_8, 11, high expression, PPRTPTSPAAT (SEQ ID NO: 553); SCAF1, In, C_8, 11, essential, PPHPHRAGCHV (SEQ ID NO: 554); SAFB2, In, C_8, 12, high expression, PPPVPPLHPPLL (SEQ ID NO: 555); PRCC, In, C_8, 13, driver, essential, PPPGNCPRCLLHR (SEQ ID NO: 556); LRP2, In, C_8, 14, high expression, PPPMQVHARRKLLF (SEQ ID NO: 557); ZC3H18, In, C_8, 15, essential, PPQEADAKRQRQWQW (SEQ ID NO: 558); MAZ, Del, C_8, 16, high expression, PPSPCWAWTPGGWAAS (SEQ ID NO: 559); ISG20L2, Del, C_8, 16, essential, PPSTGRLTARRMPPCL (SEQ ID NO: 560); NKTR, In, C_8, 17, high expression, PPPFKMEAWTETLEALL (SEQ ID NO: 561); FNDC4, Del, C_8, 18, high expression, PPADSVGTWLRWCPFPHI (SEQ ID NO: 562); ZSWIM8, Del, C_8, 18, essential, PPTLMMSNGCWGWQQSWE (SEQ ID NO: 563); SMARCC2, Del, C_8, 19, high expression, PPLDPMAPHRSPTNKLLPQ (SEQ ID NO: 564); RAPH1, Del, C_8, 20, high expression, PPHLLLSLHPSLPKLPQNPL (SEQ ID NO: 565); LARP1, In, C_8, 20, high expression, PPHPSLATRDQTGACLARPG (SEQ ID NO: 566); TAF1C, In, C_8, 20, essential, PPPVSSFQDRLPPCISSAGA (SEQ ID NO: 567); NKTR, Del, C_8, 22, high expression, PPLQDGSLDRNLGSPLMSEFRK (SEQ ID NO: 568); SRCAP, In, C_8, 23, essential, PPPTTSFPFLSGLPGGKAEAAAV (SEQ ID NO: 569); LARP1, Del, C_8, 24, high expression, PPPQPGNQRSNRSLPGTTRMRHRV (SEQ ID NO: 570); BRD3, Del, C_8, 25, driver, PPRRSPTPASPAARPTSCSTCRMWW (SEQ ID NO: 571); HNF1A, In, C_8, 27, driver, PPPRARPGTCAARSQLPWPASTCPLPQ (SEQ ID NO: 572); FLCN, In, C_8, 28, driver, PPPRALLRVCCHRGGPRSRTFHPPPCGV (SEQ ID NO: 573); FNDC4, In, C_8, 29, high expression, PPQRTPWGHGFAGAPFPISKPHGPPAGQL (SEQ ID NO: 574); SCAF1, Del, C_8, 29, essential, PPPPPGWLPRLTRERAAAALRAVGTQISI (SEQ ID NO: 575); TAF1C, Del, C_8, 29, essential, PPSLFLPGSTPSLHFLCWSLRSSGGCRSA (SEQ ID NO: 576); BAG6, In, C_8, 31, essential, PPPNSQCRGTSPWSWAGLIRGSVFYQCRVLS (SEQ ID NO: 577); MAZ, In, C_8, 35, high expression, PPLPRAGPGLPGGGRPHELLPATSGSRPEPPAGRG (SEQ ID NO: 578); ATP2A1, In, C_8, 40, high expression, PPPEPQGAPHQWLALLPLHGNRGLCGCSHRGSSCLVVPVR (SEQ ID NO: 580); STXS, In, C_8, 40, essential, PPPSDPRPSPSRHHVLPGSDPGVSVCLQVAADPSEWNPDK (SEQ ID NO: 581); FLCN, Del, C_8, 40, driver, PPTCSPQSLLSSWRSTQPHVPPSTLWGVRMTSLSASTSLW (SEQ ID NO: 582); BPTF, In, C_8, 44, essential, high expression, PPQHQRPGPGGARRRGRQDGGRGRRRPPGPDHRGPEGRQQSGVR (SEQ ID NO: 583); LRP2, Del, C_8, 47, high expression, PPHAGACTEEIAILMRLTSPNASVLAATPENIVKWRFQKASLQEQPQ (SEQ ID NO: 584); PRCC, Del, C_8, 48, driver, essential, PPRKLPQMPPSSMTKHLSGCRARGTEGEKKSTLWRSKVMTSSVGPSNG (SEQ ID NO: 585); YBX1, Del, C_8, 49, essential, high expression, PPPPPPSAPPTPSPALRAAAQGAVARAASHRRRLPAGTRRSSQRRFWEQ (SEQ ID NO: 586); ATP2A1, Del, C_8, 50, high expression, PPGAPRSPSSVAGSSSATWQSGAMWVQPPWEQLPGGSCTLRMGLMSTTAS (SEQ ID NO: 587); HNF1A, Del, C_8, 52, driver, PPQGQARDLRCPLTAPLACLHLPSPPVRSTVCAMDSLRPVRLQKYPQAAAVP (SEQ ID NO: 588); BRD3, In, C_8, 53, driver, PPPGGLQPQQARPQDQPAAVHAECGGEDALETPVRLALLPARGRNQIEPAGLS (SEQ ID NO: 589); HNRNPL, In, C_8, 58, essential, high expression, PPPTSLRREKDGSTSGGSPSGPKSLRPPVWAPPTPSPTTRVWPSRRQPCAHGLWLGSI (SEQ ID NO: 590); HNRNPL, Del, C_8, 59, essential, high expression, PPHLTTKGEGWVHQWGVTVGAQVATAPSMGTPHPLPHHPSMALTPTALCSWSMAWINLR (SEQ ID NO: 591); SMARCC2, In, C_8, 60, high expression, PPPWTPWPLTVPQPTNSSLNDARGSARQRAPRRGGPRHPPASRPHSPEPRHGHPCATSTV (SEQ ID NO: 592); BAG6, Del, C_8, 62, essential, PPQLPMQRHLPLVLGRPHPWLRLLPMSSPQLRGLPRQVQLPRQPPATRGSSGFPTRVWNPWS (SEQ ID NO: 593); YBX1, In, C_8, 65, essential, high expression, PPRRPRPQRRRHQARHYGQRRRERWPGRPHIGGACRRGQEGHRNEGFGNSKMVQCKERIWFHQQ E (SEQ ID NO: 594); DIAPH1, In, C_8, 73, high expression, PPPTSSLAWRSRNATSSSPSSWWSWNPSTSSISRRPWHSSTSTRNGYASTSPIWIWSSCSPSSAIWINP QKAL (SEQ ID NO: 596); PPRC1, In, C_8, 77, essential, PPPTNGAPSVWYSWCLCRASHLQCALGTPSCPSLTLQFHMYLWALGMGPRASTCSILVYCSPTSFA SSLHWESCSPT (SEQ ID NO: 597); SMARCC2, In, C_8, 82, high expression, PPPWTPWPLTVPQPTNSSLNDARGSARQRAPRRGGPKPCHCGSCSGQPPAQCQPTARPRHPPASRP HSPEPRHGHPCATSTV (SEQ ID NO: 598); SRCAP, Del, C_8, 90, essential, PPHHLVPLFIWTPWRKSGSGSGLNAWNGFSNLVRLMGPWHLCMGLKSWISVPCPNLLPAPSALVL LAPATPPFGLIPRLPTGLYCFSPSD (SEQ ID NO: 599); MEPCE, In, G_8, 9, essential, GGGRGQEEK (SEQ ID NO: 600); MEPCE, Del, G_8, 12, essential, GGAGARGEIAVM (SEQ ID NO: 601); ASXL1, In, G_8, 14, driver, GGGWPGWRWRRGHR (SEQ ID NO: 602); GLYR1, In, G_8, 16, high expression, GGALSGSPRLRESAAV (SEQ ID NO: 603); SRRT, In, G_8, 17, essential, high expression, GGGWGPNLWPPSALGPP (SEQ ID NO: 604); MAZ, Del, G_8, 18, high expression, GGPPPLSRETPPSHSPPA (SEQ ID NO: 605); MAZ, In, G_8, 19, high expression, GGGRPPCPGRRPPATAHLL (SEQ ID NO: 606); GLYR1, Del, G_8, 21, high expression, GGAFWKPPSQGISSCLMTGCW (SEQ ID NO: 607); IGF2R, Del, G_8, 28, high expression, GGTLAIRFISAPQPSSSTVTAAPSGQYF (SEQ ID NO: 608); BPTF, In, G_8, 35, essential, high expression, GGARRRGRQDGGRGRRRPPGPDHRGPEGRQQSGVR (SEQ ID NO: 609); TFE3, Del, G_8, 45, driver, GGDLPRMLPISSPLHRPQMPFWTCTFPATTWGTWETPSTWGWRTF (SEQ ID NO: 610); SRRT, Del, G_8, 46, essential, high expression, GGVGAQLMAPLSPGATLTSTSCSTMSCLSRPGWAALQRLTWVCRRP (SEQ ID NO: 611); ASXL1, Del, G_8, 59, driver, GGVARVEVAAGPPMREVAEAAAVVMVVRPVATLSPGEARAPLESVRQIYSEHNYCRLIL (SEQ ID NO: 612); SHC1, In, G_8, 62, high expression, GGGGRHEASGRSRSRGCSTHCTQCPDPQPLGSYIACRTACWGRSRSPQTDATSTTLSRQRAF (SEQ ID NO: 613); SHC1, In, G_8, 63, high expression, GGGGRHEASGRSRSRGCSTHCTQCPDPQPLGSYIACRTACWGRSRSPQTDATSTTLSSRQRAF (SEQ ID NO: 614); TFE3, In, G_8, 97, driver, GGGTCPECSPSAAPCTALRCPSGPALSQRPPGGPGRPLPPGAGGHSDGGGGGGGGRTVGGCPVPTA GCLRSPALFSVPCCLQGQQPPQQLQHGRGVL (SEQ ID NO: 615); PDCD6IP, In, U_8, 8, high expression, FFFKSHKR (SEQ ID NO: 616); SPG20, In, U_8, 9, high expression, FFCKSCRGG (SEQ ID NO: 617); MYO9A, Del, U_8, 9, high expression, FFISPNKTH (SEQ ID NO: 618); FBXO21, Del, U_8, 9, high expression, FLRMNWCVS (SEQ ID NO: 619); BDP1, In, U_8, 10, essential, high expression, FFFSHQHGRN (SEQ ID NO: 620); GOLGA2, Del, U_8, 10, high expression, FFTGLTRMMR (SEQ ID NO: 623); TRIO, In, U_8, 10, essential, FFFRRVREVP (SEQ ID NO: 624); DDX39B, In, U_8, 10, essential, FFWWSVYQEG (SEQ ID NO: 625); KIAA1429, Del, U_8, 11, essential, high expression, FLCRNMKQQIY (SEQ ID NO: 626); PREX2, Del, U_8, 11, driver, FFRMRKWRDQI (SEQ ID NO: 627); HNRNPH1, In, U_8, 12, essential, high expression, FFFTAQPCESTH (SEQ ID NO: 628); PDLIM5, Del, U_8, 12, high expression, FLQQFTLRSFSI (SEQ ID NO: 629); CANX, Del, U_8, 12, high expression, FLTTLSFVLIEE (SEQ ID NO: 630); PLK2, In, U_8, 13, high expression, FFFAGLHSGQTVF (SEQ ID NO: 631); DSTN, In, U_8, 13, high expression, FFFVGTRTSTSEK (SEQ ID NO: 632); PAXIP1, In, U_8, 13, essential, FFWNHCLPFSGVI (SEQ ID NO: 633); DDX39B, Del, U_8, 13, essential, FLVVCLSRRMKRC (SEQ ID NO: 634); VPS33A, In, U_8, 14, essential, FFCQTQARVDGYNR (SEQ ID NO: 635); DMXL2, In, U_8, 16, high expression, FFFEFCDIQLSMGSSR (SEQ ID NO: 636); ZCCHC6, Del, U_8, 17, high expression, FFMNSVNLSSPKASLLR (SEQ ID NO: 637); TMEM41B, In, U_8, 17, essential, FFYWYFSRCRTSFFCSH (SEQ ID NO: 638); XRCC2, Del, U_8, 18, essential, FFWCTAVVAPTYFLHFTH (SEQ ID NO: 639); TRAPPC8, Del, U_8, 18, essential, FLAMTDDQRMVKNKQLLM (SEQ ID NO: 640); OGDH, Del, U_8, 20, essential, high expression, FFATRMPEPHRALPTRVPFP (SEQ ID NO: 641); SPATA5L1, Del, U_8, 23, essential, FFLELILETSAQKLLCWLCKKMD (SEQ ID NO: 642); IPO11, Del, U_8, 23, essential, FFLHYLMRWPINLIRRWTSFWEI (SEQ ID NO: 643); EXOSC9, Del, U_8, 23, essential, FLTLNSLRWPLQLSNLAGSQISW (SEQ ID NO: 644); ZCCHC6, Del, U_8, 24, high expression, FFMNSVNLSSPKARDWTVSELLKN (SEQ ID NO: 645); HLA-F, In, U_8, 25, high expression, FFFVAVDIFQHCLLGLAKPSLWLRL (SEQ ID NO: 646); AGT, Del, U_8, 25, high expression, FLSLKRMRESPQSLPNSLTSLRSWR (SEQ ID NO: 647); MYO9A, In, U_8, 28, high expression, FFLYPPTRPTENKFPTRHKYPKKQTIGK (SEQ ID NO: 648); DPAGT1, Del, U_8, 29, essential, FFSPLWDCSTTTGTHHGCLWEIPSVTLLA (SEQ ID NO: 650); XRN1, In, U_8, 31, essential, high expression, FFFEEKWCSSIPAKQSWRKHDVGNLSGCRIR (SEQ ID NO: 651); PAXIP1, Del, U_8, 31, essential, FLESLPAFLRCHLKTEVPCGLWLRSMGEIAS (SEQ ID NO: 652); ATP13A3, Del, U_8, 32, high expression, FFGSNSNLGMKCGIQNQMLVIQQEAGFGILHT (SEQ ID NO: 653); INTS2, Del, U_8, 34, essential, FFSSLLNFLRVQYIWRKLQMYFVFYKQSSLPCSL (SEQ ID NO: 654); HLA-F, Del, U_8, 39, high expression, FFCGCGHLSTLPSWPCKAFALASALGGAGASFFVLGTIL (SEQ ID NO: 655); TMEM57, Del, U_8, 47, high expression, FLLLAHMYGFSTYGTQKGECVCLQCLSGSSLFILKQPLDLKISKTFM (SEQ ID NO: 656); PJA1, Del, U_8, 59, high expression, FLILMMMTICHTVLPGGGIPPMTMRATRMAWQEEGEARVQVAIPSQSTLKTNGKRGVTK (SEQ ID NO: 657); HDGF, In, U_8, 65, high expression, FFFRDPRDGIPGPQRPLPLRGIQGEVWQAQQEERVQRGAVGDREQPYCQGFRLSVLPEKELCGRA (SEQ ID NO: 658); PLK2, Del, U_8, 69, high expression, FFCRASLRTDCLLAVVIQFQISTYQAQLRISLRKQLLLFLVAKKTKQDILTHIIECLKKMKTSTSLGMI (SEQ ID NO: 660); OGDH, In, U_8, 77, essential, high expression, FFSQHECRSPTGHCLPESPSPEPRLPGCCGPCTVPGRSTAQRGQARGGPPGSAVAHQGISDTRAPCST AGPPGDFGC (SEQ ID NO: 661); ATRX, In, A_7, 8, driver, essential, high expression, KKKNWKTK (SEQ ID NO: 662); SPEN, In, A_7, 8, driver, essential, high expression, KKNQTGQT (SEQ ID NO: 663); DDX10, In, A_7, 8, driver, essential, high expression, KKSNEEKF (SEQ ID NO: 664); CDC5L, In, A_7, 8, essential, high expression, KKACPWFL (SEQ ID NO: 665); IWS1, Del, A_7, 8, essential, high expression, KKADLHTQ (SEQ ID NO: 666); MRPS31, In, A_7, 8, essential, high expression, KKGYSKRK (SEQ ID NO: 667); CTNNBL1, In, A_7, 8, essential, high expression, KKKAAADY (SEQ ID NO: 668); NOP14, In, A_7, 8, essential, high expression, KKKHLQSK (SEQ ID NO: 669); RBM39, In, A_7, 8, essential, high expression, KKKQEQKS (SEQ ID NO: 670); EIF5B, Del, A_7, 8, essential, high expression, KKLKSVQE (SEQ ID NO: 671); NIPBL, In, A_7, 8, essential, high expression, KKNCSNEC (SEQ ID NO: 672); TFAM, In, A_7, 8, essential, high expression, KKNISRCL (SEQ ID NO: 673); SENP6, In, A_7, 8, essential, high expression, KKNKSYCE (SEQ ID NO: 674); NMD3, In, A_7, 8, essential, high expression, KKNQSPSE (SEQ ID NO: 675); DNTTIP2, In, A_7, 8, essential, high expression, KKPLCSTI (SEQ ID NO: 676); USP9X, Del, A_7, 8, essential, high expression, KKPPVCSR (SEQ ID NO: 677); MRPSS, In, A_7, 8, essential, high expression, KKRKRQKN (SEQ ID NO: 678); PSMC2, In, A_7, 8, essential, high expression, KKSLPYLL (SEQ ID NO: 679); CYLD, Del, A_7, 8, driver, high expression, KKFFLLWN (SEQ ID NO: 680); PSIP1, In, A_7, 8, driver, high expression, KKNTAIQS (SEQ ID NO: 681); IDH1, Del, A_7, 8, driver, high expression, KKSVAVLW (SEQ ID NO: 682); PPFIBP1, Del, A_7, 8, driver, high expression, KSLNQPNL (SEQ ID NO: 683); SCAPER, Del, A_7, 8, high expression, KIKKKPKR (SEQ ID NO: 684); AKAP11, Del, A_7, 8, high expression, KINLKINP (SEQ ID NO: 685); ARHGAP21, Del, A_7, 8, high expression, KIRWNQET (SEQ ID NO: 686); RSF1, In, A_7, 8, high expression, KKDYQRRK (SEQ ID NO: 687); FEM1C, Del, A_7, 8, high expression, KKEICLGL (SEQ ID NO: 688); ZCRB1, In, A_7, 8, high expression, KKEKKESS (SEQ ID NO: 689); POMP, In, A_7, 8, high expression, KKFPAQPR (SEQ ID NO: 690); ZCCHC6, In, A_7, 8, high expression, KKFTCGSV (SEQ ID NO: 691); TTN, In, A_7, 8, high expression, KKGSSKSH (SEQ ID NO: 692); ITPR2, Del, A_7, 8, high expression, KKGTMTMN (SEQ ID NO: 693); UBR1, In, A_7, 8, high expression, KKHRGQVP (SEQ ID NO: 694); ZNF652, In, A_7, 8, high expression, KKHSVCFL (SEQ ID NO: 695); PHF3, Del, A_7, 8, high expression, KKHYRIKL (SEQ ID NO: 696); DDX50, In, A_7, 8, high expression, KKIHHQI (SEQ ID NO: 697); FAM135A, In, A_7, 8, high expression, KKINKTEL (SEQ ID NO: 698); RSF1, In, A_7, 8, high expression, KKIQSKLA (SEQ ID NO: 699); TTC3, In, A_7, 8, high expression, KKKEEEKH (SEQ ID NO: 700); KRCC1, In, A_7, 8, high expression, KKKPRCSL (SEQ ID NO: 701); RSF1, In, A_7, 8, high expression, KKKRGRRR (SEQ ID NO: 702); EPSTI1, In, A_7, 8, high expression, KKKTSRKP (SEQ ID NO: 703); BAZ1A, Del, A_7, 8, high expression, KKLLKKRD (SEQ ID NO: 704); ZNF564, In, A_7, 8, high expression, KKMGRPEH (SEQ ID NO: 705); MAP4K4, In, A_7, 8, high expression, KKMVEEVF (SEQ ID NO: 706); TAF7, In, A_7, 8, high expression, KKNFLQDS (SEQ ID NO: 707); RUFY3, In, A_7, 8, high expression, KKNFSRTK (SEQ ID NO: 708); LMOD3, In, A_7, 8, high expression, KKNIEIRS (SEQ ID NO: 709); ADAMTS1, In, A_7, 8, high expression, KKNIRISY (SEQ ID NO: 710); SMARCA2, In, A_7, 8, high expression, KKNKGKDS (SEQ ID NO: 711); IFI16, In, A_7, 8, high expression, KKNKPEKQ (SEQ ID NO: 712); PDZD8, In, A_7, 8, high expression, KKNKQVHR (SEQ ID NO: 713); CCDC88A, In, A_7, 8, high expression, KKNPAIRE (SEQ ID NO: 714); VPS13B, In, A_7, 8, high expression, KKNRGPLS (SEQ ID NO: 715); PHF20L1, In, A_7, 8, high expression, KKNSSRHS (SEQ ID NO: 716); ATE1, In, A_7, 8, high expression, KKNVEISS (SEQ ID NO: 717); RB1CC1, In, A_7, 8, high expression, KKPKRTNN (SEQ ID NO: 718); CCDC66, Del, A_7, 8, high expression, KKPSGGKS (SEQ ID NO: 719); MPRIP, In, A_7, 8, high expression, KKQEQLLF (SEQ ID NO: 720); PLEKHA5, In, A_7, 8, high expression, KKQGNEQN (SEQ ID NO: 721); CHD9, Del, A_7, 8, high expression, KKQKHVLS (SEQ ID NO: 722); GOLGB1, In, A_7, 8, high expression, KKRAGGRR (SEQ ID NO: 723); LARP4, In, A_7, 8, high expression, KKSCEASV (SEQ ID NO: 724); HIST1H1D, In, A_7, 8, high expression, KKSCQESS (SEQ ID NO: 725); RPS6KA3, Del, A_7, 8, high expression, KKSPQQPC (SEQ ID NO: 726); SNX25, In, A_7, 8, high expression, KKSPVAFS (SEQ ID NO: 727); FAM98A, In, A_7, 8, high expression, KKSSRRRR (SEQ ID NO: 728); SYNE2, In, A_7, 8, high expression, KKTGSAHF (SEQ ID NO: 729); SLTM, Del, A_7, 8, high expression, KKVKKRSE (SEQ ID NO: 730); ZNF280D, Del, A_7, 8, high expression, KKVKSIQH (SEQ ID NO: 731); REST, Del, A_7, 8, high expression, KKVQRRKL (SEQ ID NO: 732); UHRF1BP1L, Del, A_7, 8, high expression, KMDFCSAT (SEQ ID NO: 733); CTSH, Del, A_7, 8, high expression, KMGSLTGS (SEQ ID NO: 734); SCG2, Del, A_7, 8, high expression, KMNKSTMR (SEQ ID NO: 735); TTC14, Del, A_7, 8, high expression, KNNPHLGL (SEQ ID NO: 736); VCAN, Del, A_7, 8, high expression, KNPLKILS (SEQ ID NO: 737); IFI16, Del, A_7, 8, high expression, KNTRTLFY (SEQ ID NO: 738); BAZ2B, Del, A_7, 8, high expression, KSGQILSK (SEQ ID NO: 739); TAF1D, Del, A_7, 8, high expression, KTHLGEKF (SEQ ID NO: 740); EEA1, Del, A_7, 8, high expression, KTIHYRSK (SEQ ID NO: 741); BRCA2, Del, A_7, 8, driver, essential, KKKKIQLY (SEQ ID NO: 742); PALB2, In, A_7, 8, driver, essential, KKNDCLYR (SEQ ID NO: 743); MYB, Del, A_7, 8, driver, essential, KNGARPAP (SEQ ID NO: 744); POLR3B, Del, A_7, 8, essential, KKAEPIWL (SEQ ID NO: 745); SPRTN, Del, A_7, 8, essential, KKAKERQN (SEQ ID NO: 746); HSPA14, In, A_7, 8, essential, KKCSWRSS (SEQ ID NO: 747); NMT1, In, A_7, 8, essential, KKEERKRQ (SEQ ID NO: 748); CMTR1, Del, A_7, 8, essential, KKELQSLP (SEQ ID NO: 749); ERCC6L, In, A_7, 8, essential, KKEPFPIF (SEQ ID NO: 750); CDC40, Del, A_7, 8, essential, KKFLRAIW (SEQ ID NO: 751); CENPK, Del, A_7, 8, essential, KKHLKQFP (SEQ ID NO: 752); GGPS1, In, A_7, 8, essential, KKILCTLS (SEQ ID NO: 753); NRDE2, In, A_7, 8, essential, KKKEASAS (SEQ ID NO: 754); CENPE, Del, A_7, 8, essential, KKLMKWNS (SEQ ID NO: 755); TRIO, In, A_7, 8, essential, KKMCYCCG (SEQ ID NO: 756); UTP20, In, A_7, 8, essential, KKNEETQK (SEQ ID NO: 757); ASCC3, In, A_7, 8, essential, KKNFRRKC (SEQ ID NO: 758); NCAPG, In, A_7, 8, essential, KKNQNTSL (SEQ ID NO: 759); CDC40, In, A_7, 8, essential, KKSRFNCI (SEQ ID NO: 760); NR2C2AP, In, A_7, 8, essential, KKTSFRPG (SEQ ID NO: 761); CENPC, Del, A_7, 8, essential, KKVFQHCF (SEQ ID NO: 762); GTF3C1, Del, A_7, 8, essential, KKVIIKWA (SEQ ID NO: 763); NDC80, Del, A_7, 8, essential, KKWVWRIL (SEQ ID NO: 764); NUP153, In, A_7, 8, essential, KKYDTRTK (SEQ ID NO: 765); HSPA14, Del, A_7, 8, essential, KMGNYDMK (SEQ ID NO: 766); PNPT1, Del, A_7, 8, essential, KNFRLKQV (SEQ ID NO: 767); CHD8, Del, A_7, 8, essential, KNIQRTWI (SEQ ID NO: 768); PLAG1, In, A_7, 8, driver, KKAPVRTL (SEQ ID NO: 769); CASP8, Del, A_7, 8, driver, KNLSSLLI (SEQ ID NO: 770); SETD2, In, A_7, 9, driver, essential, high expression, KKKEAGNRE (SEQ ID NO: 771); BDP1, Del, A_7, 9, essential, high expression, KIVCRKGVN (SEQ ID NO: 772); SDAD1, In, A_7, 9, essential, high expression, KKEEKTRGV (SEQ ID NO: 773); RPS6, Del, A_7, 9, essential, high expression, KKERRIFLD (SEQ ID NO: 774); SHOC2, In, A_7, 9, essential, high expression, KKIQQCRGD (SEQ ID NO: 775); MEF2C, Del, A_7, 9, essential, high expression, KKLMKNLII (SEQ ID NO: 776); NMD3, Del, A_7, 9, essential, high expression, KKLSTIWNS (SEQ ID NO: 777); TCERG1, In, A_7, 9, essential, high expression, KKNDGRSKI (SEQ ID NO: 778); ZNF131, In, A_7, 9, essential, high expression, KKNSCMSVL (SEQ ID NO: 779); EIF5B, In, A_7, 9, essential, high expression, KKNTTAARK (SEQ ID NO: 780); SMNDC1, In, A_7, 9, essential, high expression, KKRNDCPAA (SEQ ID NO: 781); ABCF1, Del, A_7, 9, essential, high expression, KKRSLKSWQ (SEQ ID NO: 782); ZC3H13, In, A_7, 9, essential, high expression, KKRTEKEKH (SEQ ID NO: 783); GNL3, Del, A_7, 9, essential, high expression, KRFENIIEN (SEQ ID NO: 784); MPHOSPH10, Del, A_7, 9, essential, high expression, KSRGSFRGK (SEQ ID NO: 785); DDX42, Del, A_7, 9, essential, high expression, KTFTMSMKR (SEQ ID NO: 786); PRRX1, In, A_7, 9, driver, high expression, KKEEKAAKE (SEQ ID NO: 787); PBRM1, In, A_7, 9, driver, high expression, KKNILYEKG (SEQ ID NO: 788); IL7R, In, A_7, 9, driver, high expression, KKNRPNHYS (SEQ ID NO: 789); MSH6, In, A_7, 9, driver, high expression, KKTKGHIRF (SEQ ID NO: 790); KIF5B, Del, A_7, 9, driver, high expression, KKWKKMKRS (SEQ ID NO: 791); TRIP11, Del, A_7, 9, driver, high expression, KKWNNLRIK (SEQ ID NO: 792); RNF213, Del, A_7, 9, driver, high expression, KTRKQMSRK (SEQ ID NO: 793); APOB, Del, A_7, 9, high expression, KIHWSLVME (SEQ ID NO: 794); TMEM126B, Del, A_7, 9, high expression, KILTILEKK (SEQ ID NO: 795); POMP, Del, A_7, 9, high expression, KISSSTKIK (SEQ ID NO: 796); ANK3, In, A_7, 9, high expression, KKDAVRVVR (SEQ ID NO: 797); CCDC47, Del, A_7, 9, high expression, KKEQRRSES (SEQ ID NO: 798); LRRK2, In, A_7, 9, high expression, KKFSFGWNR (SEQ ID NO: 799); CHD9, Del, A_7, 9, high expression, KKGEEGGRM (SEQ ID NO: 800); CFI, In, A_7, 9, high expression, KKIYSPLLR (SEQ ID NO: 801); FXR1, Del, A_7, 9, high expression, KKKLVKEMK (SEQ ID NO: 802); FUT8, In, A_7, 9, high expression, KKKSVFGHR (SEQ ID NO: 803); SYNE2, Del, A_7, 9, high expression, KKLKQSWKC (SEQ ID NO: 804); GOLGA4, Del, A_7, 9, high expression, KKLLRKMMI (SEQ ID NO: 805); IFNAR1, Del, A_7, 9, high expression, KKLMLQFLI (SEQ ID NO: 806); MEF2A, Del, A_7, 9, high expression, KKLMRNLII (SEQ ID NO: 807); KLHLS, In, A_7, 9, high expression, KKNGKLFET (SEQ ID NO: 808); NEXN, Del, A_7, 9, high expression, KKNHFLPEN (SEQ ID NO: 809); RPS6KA3, In, A_7, 9, high expression, KKNHLNSPV (SEQ ID NO: 810); EHBP1, In, A_7, 9, high expression, KKNKFISYF (SEQ ID NO: 811); TXLNB, In, A_7, 9, high expression, KKNQRRRNI (SEQ ID NO: 812); TBC1D1, In, A_7, 9, high expression, KKNSIGEKF (SEQ ID NO: 813); PIK3CB, In, A_7, 9, high expression, KKNWPALPH (SEQ ID NO: 814); CTSK, In, A_7, 9, high expression, KKPEVYFHP (SEQ ID NO: 815); TCF25, In, A_7, 9, high expression, KKRPLLLCV (SEQ ID NO: 816); HMGNS, Del, A_7, 9, high expression, KKRSHRVLF (SEQ ID NO: 817); EPB41L5, In, A_7, 9, high expression, KKSQRTRVV (SEQ ID NO: 818); SLC39A_8, Del, A_7, 9, high expression, KKSQVHVPV (SEQ ID NO: 819); GOLGB1, In, A_7, 9, high expression, KKSYVFTPK (SEQ ID NO: 820); ARHGAPS, In, A_7, 9, high expression, KKTCNNSSN (SEQ ID NO: 821); ZNF737, In, A_7, 9, high expression, KKTFDHEET (SEQ ID NO: 822); ZNF506, In, A_7, 9, high expression, KKTFNYEEA (SEQ ID NO: 823); ZNF253, In, A_7, 9, high expression, KKTFNYGKT (SEQ ID NO: 824); TRIP12, In, A_7, 9, high expression, KKTKSTSAY (SEQ ID NO: 825); CPVL, In, A_7, 9, high expression, KKVLPEAVP (SEQ ID NO: 826); ANKRD12, Del, A_7, 9, high expression, KKWIKKVKH (SEQ ID NO: 827); CCDC66, In, A_7, 9, high expression, KKWNDFHH (SEQ ID NO: 828); TMEM131, In, A_7, 9, high expression, KKYNIKNHC (SEQ ID NO: 829); ZNF292, Del, A_7, 9, high expression, KMKKMKWMN (SEQ ID NO: 830); HEBP2, Del, A_7, 9, high expression, KMNPPKKTN (SEQ ID NO: 831); IQGAP2, Del, A_7, 9, high expression, KNQVIFCLY (SEQ ID NO: 832); TM9SF2, Del, A_7, 9, high expression, KRATSARPK (SEQ ID NO: 833); FKBP15, Del, A_7, 9, high expression, KRKQSCRCS (SEQ ID NO: 834); ALMS1, Del, A_7, 9, high expression, KRLTYQQDL (SEQ ID NO: 835); DMD, Del, A_7, 9, high expression, KSLKTLKSS (SEQ ID NO: 836); SYNE2, Del, A_7, 9, high expression, KSPFWMSCQ (SEQ ID NO: 837); IFIT1, Del, A_7, 9, high expression, KSPHLRWLI (SEQ ID NO: 838); RBM34, Del, A_7, 9, high expression, KSWQTGKAL (SEQ ID NO: 839); GCC2, Del, A_7, 9, high expression, KTPKSSNCL (SEQ ID NO: 840); NAIP, Del, A_7, 9, high expression, KTTYPGPAY (SEQ ID NO: 841); NOSTRIN, Del, A_7, 9, high expression, KTWRVILPD (SEQ ID NO: 842); ATR, In, A_7, 9, driver, essential, KKKRYHSFL (SEQ ID NO: 843); SMC2, Del, A_7, 9, essential, KIWLRTQKL (SEQ ID NO: 844); MBTPS2, Del, A_7, 9, essential, KKAQVQVSV (SEQ ID NO: 845); RFC1, In, A_7, 9, essential, KKCPRKKKN (SEQ ID NO: 846); NIFK, Del, A_7, 9, essential, KKDEEAAIS (SEQ ID NO: 847); NAA25, Del, A_7, 9, essential, KKDLRPQRN (SEQ ID NO: 848); MMS22L, Del, A_7, 9, essential, KKFSVQGCS (SEQ ID NO: 849); MCM10, In, A_7, 9, essential, KKHHVFFSS (SEQ ID NO: 850); BORA, In, A_7, 9, essential, KKIQLGKHN (SEQ ID NO: 851); NOL9, In, A_7, 9, essential, KKKERPSVN (SEQ ID NO: 852); TOP2A, In, A_7, 9, essential, KKKGCPKRN (SEQ ID NO: 853); C1orf131, In, A_7, 9, essential, KKKKIDARS (SEQ ID NO: 854); ORC2, In, A_7, 9, essential, KKNNKEARI (SEQ ID NO: 855); CHD8, In, A_7, 9, essential, KKSKVTKHF (SEQ ID NO: 856); U2SURP, In, A_7, 9, essential, KKSSKSVFQ (SEQ ID NO: 857); BOD1L1, In, A_7, 9, essential, KKTSISANH (SEQ ID NO: 858); DNMT1, Del, A_7, 9, essential, KKWLAPKQS (SEQ ID NO: 859); GEMIN5, Del, A_7, 9, essential, KMSHFLCLS (SEQ ID NO: 860); ANLN, Del, A_7, 9, essential, KNAVLTTLK (SEQ ID NO: 861); NIFK, Del, A_7, 9, essential, KNKNNLLLE (SEQ ID NO: 862); MDC1, Del, A_7, 9, essential, KNQGPHLPL (SEQ ID NO: 863); TIMM44, Del, A_7, 9, essential, KNSVTRPEG (SEQ ID NO: 864); RBM48, Del, A_7, 9, essential, KNYKCGRHM (SEQ ID NO: 865); RNASEH2B, Del, A_7, 9, essential, KRLIKLWQH (SEQ ID NO: 866); NF1, In, A_7, 9, driver, KKGKQKISG (SEQ ID NO: 867); NBN, Del, A_7, 9, driver, KKGKWMMWP (SEQ ID NO: 868); STATSB, In, A_7, 9, driver, KKTSQASLE (SEQ ID NO: 869); TRIM24, Del, A_7, 9, driver, KRKLKALLS (SEQ ID NO: 870); CNTRL, Del, A_7, 9, driver, KSWHKPKGF (SEQ ID NO: 871); ATRX, In, A_7, 10, driver, essential, high expression, KKKNSKYKRF (SEQ ID NO: 872); SETD2, In, A_7, 10, driver, essential, high expression, KKRRFPYWEG (SEQ ID NO: 873); ATRX, Del, A_7, 10, driver, essential, high expression, KRNLQTLRKK (SEQ ID NO: 874); CEP350, Del, A_7, 10, essential, high expression, KIRQKKPLYL (SEQ ID NO: 875); SMARCC1, Del, A_7, 10, essential, high expression, KIVKRNRIVK (SEQ ID NO: 876); EIF3M, In, A_7, 10, essential, high expression, KKAHPFKTTL (SEQ ID NO: 877); PSMD1, In, A_7, 10, essential, high expression, KKGGGENGSG (SEQ ID NO: 878); XRN1, In, A_7, 10, essential, high expression, KKKGCKQKEL (SEQ ID NO: 879); SEC62, In, A_7, 10, essential, high expression, KKRKREKKRW (SEQ ID NO: 880); BCAS2, Del, A_7, 10, essential, high expression, KMTLLHGKNV (SEQ ID NO: 881); NAE1, Del, A_7, 10, essential, high expression, KRTTVILHGL (SEQ ID NO: 882); RARS2, Del, A_7, 10, essential, high expression, KSIFSKYSKC (SEQ ID NO: 883); CHD1, Del, A_7, 10, essential, high expression, KTKNQERFSI (SEQ ID NO: 884); MTIF2, Del, A_7, 10, essential, high expression, KTQIMYMKLY (SEQ ID NO: 885); PSIP1, Del, A_7, 10, driver, high expression, KKYGDSKLVR (SEQ ID NO: 886); RUFY1, Del, A_7, 10, high expression, KIMWKNLTGT (SEQ ID NO: 888); IFT74, Del, A_7, 10, high expression, KIQEVSKSKF (SEQ ID NO: 889); MYO9A, Del, A_7, 10, high expression, KIQIICAQTL (SEQ ID NO: 890); PLG, Del, A_7, 10, high expression, KITAVTLMVM (SEQ ID NO: 891); S100A_8, Del, A_7, 10, high expression, KKAMKKATKS (SEQ ID NO: 892); SECISBP2L, Del, A_7, 10, high expression, KKEKRRKLQN (SEQ ID NO: 893); PSD3, In, A_7, 10, high expression, KKFTRNTSFS (SEQ ID NO: 894); NRIP1, In, A_7, 10, high expression, KKHQPSGSTQ (SEQ ID NO: 895); APPL1, In, A_7, 10, high expression, KKIFNFASRE (SEQ ID NO: 896); TMEM131, In, A_7, 10, high expression, KKIKDCKHLF (SEQ ID NO: 897); UACA, In, A_7, 10, high expression, KKISRNGKRT (SEQ ID NO: 898); APBB HP, Del, A_7, 10, high expression, KKKARKLMRK (SEQ ID NO: 899); MORF4L1, In, A_7, 10, high expression, KKKCCEAQAL (SEQ ID NO: 900); SYNE2, In, A_7, 10, high expression, KKKCFARSLF (SEQ ID NO: 901); KDM5B, In, A_7, 10, high expression, KKKHQIRESE (SEQ ID NO: 902); BRWD1, In, A_7, 10, high expression, KKKHQSYELC (SEQ ID NO: 903); ADAMTS1, In, A_7, 10, high expression, KKKKLEVSVP (SEQ ID NO: 904); CMYA5, Del, A_7, 10, high expression, KKKSQAIQRK (SEQ ID NO: 905); ASH1L, Del, A_7, 10, high expression, KKLPRETMDN (SEQ ID NO: 906); GALC, In, A_7, 10, high expression, KKMVYTHVNY (SEQ ID NO: 907); SORL1, In, A_7, 10, high expression, KKNALCFLCI (SEQ ID NO: 908); HERC1, In, A_7, 10, high expression, KKNDGKASQN (SEQ ID NO: 909); TRAF3IP1, In, A_7, 10, high expression, KKNFGDEERL (SEQ ID NO: 910); ZNF844, In, A_7, 10, high expression, KKNFSWSKIM (SEQ ID NO: 911); ZBTB38, In, A_7, 10, high expression, KKNSKWRLEA (SEQ ID NO: 912); OAT, In, A_7, 10, high expression, KKNSPRPSNL (SEQ ID NO: 913); USMG5, In, A_7, 10, high expression, KKNSSCESNI (SEQ ID NO: 914); ZNF484, In, A_7, 10, high expression, KKNSYWRETL (SEQ ID NO: 915); ZNF124, In, A_7, 10, high expression, KKNSYWRKAL (SEQ ID NO: 916); MYO9A, In, A_7, 10, high expression, KKNYPFAINM (SEQ ID NO: 917); MIA3, In, A_7, 10, high expression, KKPWESGQAV (SEQ ID NO: 918); HMGN5, In, A_7, 10, high expression, KKRGATEYCL (SEQ ID NO: 919); TP53BP1, In, A_7, 10, high expression, KKRMLRSYGS (SEQ ID NO: 920); RBL2, Del, A_7, 10, high expression, KKSGPALNSP (SEQ ID NO: 921); PTPRA, In, A_7, 10, high expression, KKSICKHLAL (SEQ ID NO: 922); RTN4, In, A_7, 10, high expression, KKSIRNKGRN (SEQ ID NO: 923); CCDC66, In, A_7, 10, high expression, KKSPVEERAR (SEQ ID NO: 924); MED13, In, A_7, 10, high expression, KKTQGRRWDI (SEQ ID NO: 925); ZNF791, In, A_7, 10, high expression, KKTYENAQSI (SEQ ID NO: 926); ADAM10, In, A_7, 10, high expression, KKYLSALYSD (SEQ ID NO: 927); CYP51A1, Del, A_7, 10, high expression, KMCLKLFLSS (SEQ ID NO: 928); ANKRD12, Del, A_7, 10, high expression, KMQFRLLVLL (SEQ ID NO: 929); RAD23A, Del, A_7, 10, high expression, KMRTWLPTSS (SEQ ID NO: 930); MAP4K4, Del, A_7, 10, high expression, KNGRRSFLVL (SEQ ID NO: 931); EHHADH, Del, A_7, 10, high expression, KNWLLRETLP (SEQ ID NO: 932); SPAG9, Del, A_7, 10, high expression, KRTYRPEWNL (SEQ ID NO: 934); BRD4, In, A_7, 10, driver, essential, KKKQSQGTSS (SEQ ID NO: 935); PIK3CA, In, A_7, 10, driver, essential, KKNSKYVAIL (SEQ ID NO: 936); TOPBP1, Del, A_7, 10, essential, KIKSNDLEYT (SEQ ID NO: 937); USP36, Del, A_7, 10, essential, KKESRRHSGQ (SEQ ID NO: 938); CEP55, Del, A_7, 10, essential, KKILRLNDKP (SEQ ID NO: 939); PPAT, In, A_7, 10, essential, KKIWSIVRQL (SEQ ID NO: 940); NIFK, In, A_7, 10, essential, KKKTKKQQSV (SEQ ID NO: 941); DIMT1, In, A_7, 10, essential, KKLQNSLFSP (SEQ ID NO: 942); MASTL, Del, A_7, 10, essential, KKLYRIKKLV (SEQ ID NO: 943); MIS18BP1, Del, A_7, 10, essential, KKNIKCLQRN (SEQ ID NO: 944); SMC2, Del, A_7, 10, essential, KKPLRKVRRL (SEQ ID NO: 945); TTK, Del, A_7, 10, essential, KKVNNFFKKL (SEQ ID NO: 946); ORC4, Del, A_7, 10, essential, KKVTELAVAQ (SEQ ID NO: 947); GGPS1, Del, A_7, 10, essential, KNTVYIILRM (SEQ ID NO: 949); ATAD5, Del, A_7, 10, essential, KNVLPLFLIV (SEQ ID NO: 950); CDC23, Del, A_7, 10, essential, KRRTMKQLIA (SEQ ID NO: 951); NRG1, In, A_7, 10, driver, KKAREVRTSH (SEQ ID NO: 952); USP6, In, A_7, 10, driver, KKTAEGSLWT (SEQ ID NO: 953); DDX10, Del, A_7, 11, driver, essential, high expression, KKLRQSIGRKD (SEQ ID NO: 954); WDR43, In, A_7, 11, essential, high expression, KKKEIRSCRNE (SEQ ID NO: 955); SOS1, In, A_7, 11, essential, high expression, KKNCKRQWTRS (SEQ ID NO: 956); CCT2, In, A_7, 11, essential, high expression, KKNWSKSTKTN (SEQ ID NO: 957); HECTD1, In, A_7, 11, essential, high expression, KKNYNKNITAD (SEQ ID NO: 958); FNBP4, In, A_7, 11, essential, high expression, KKRKERQGKEE (SEQ ID NO: 959); MRPL15, In, A_7, 11, essential, high expression, KKWWCCYYSLL (SEQ ID NO: 961); HSP90AA1, Del, A_7, 11, driver, high expression, KKQSLRTSAKS (SEQ ID NO: 962); TDO2, Del, A_7, 11, high expression, KISPEAWKRNS (SEQ ID NO: 963); PSD3, Del, A_7, 11, high expression, KIYQKYLFQLK (SEQ ID NO: 964); CPNE3, Del, A_7, 11, high expression, KKATRIQVLSV (SEQ ID NO: 965); LONP2, Del, A_7, 11, high expression, KKCLSQCQNML (SEQ ID NO: 966); FAM13B, In, A_7, 11, high expression, KKETTQNVAGI (SEQ ID NO: 967); WDR60, In, A_7, 11, high expression, KKGNTRNSKSY (SEQ ID NO: 968); TTC3, Del, A_7, 11, high expression, KKGRRKTLKQK (SEQ ID NO: 969); PIK3C2A, Del, A_7, 11, high expression, KKHLYYQLLLF (SEQ ID NO: 970); CD46, In, A_7, 11, high expression, KKILLQSNSYV (SEQ ID NO: 971); ERICH1, In, A_7, 11, high expression, KKKETEKETAN (SEQ ID NO: 972); ROCK2, In, A_7, 11, high expression, KKKKSKLSNIH (SEQ ID NO: 973); SP110, In, A_7, 11, high expression, KKKPPRRDSLI (SEQ ID NO: 974); CCDC47, In, A_7, 11, high expression, KKKSREGANHE (SEQ ID NO: 975); REST, In, A_7, 11, high expression, KKKYKEENSEK (SEQ ID NO: 976); PARP14, Del, A_7, 11, high expression, KKLWMPRMARQ (SEQ ID NO: 977); P4HA1, In, A_7, 11, high expression, KKMGREVRSAN (SEQ ID NO: 978); ARID4B, In, A_7, 11, high expression, KKNAERAFFRL (SEQ ID NO: 979); DENND4A, In, A_7, 11, high expression, KKNAVKEDGST (SEQ ID NO: 980); ATF4, In, A_7, 11, high expression, KKNGAKQDSSH (SEQ ID NO: 981); UBE2K, In, A_7, 11, high expression, KKNRKPMCYGL (SEQ ID NO: 982); ANKRD12, In, A_7, 11, high expression, KKNRRKTQITV (SEQ ID NO: 983); NASP, In, A_7, 11, high expression, KKNRRQVFGKA (SEQ ID NO: 984); VPS13B, In, A_7, 11, high expression, KKNSLRVLNSS (SEQ ID NO: 985); ZHX3, In, A_7, 11, high expression, KKNSYTQGECP (SEQ ID NO: 986); SRPK1, In, A_7, 11, high expression, KKNYSASVTGS (SEQ ID NO: 987); UPF3A, In, A_7, 11, high expression, KKRCQDWKHRR (SEQ ID NO: 988); ZEB1, In, A_7, 11, high expression, KKRKSSRYKQL (SEQ ID NO: 989); PDS5B, Del, A_7, 11, high expression, KKRPSQNRRRN (SEQ ID NO: 990); TOP2B, In, A_7, 11, high expression, KKRRVRNCQQK (SEQ ID NO: 991); TAF1D, In, A_7, 11, high expression, KKRTLEKNFNV (SEQ ID NO: 992); PHF3, In, A_7, 11, high expression, KKSIWEDCIYC (SEQ ID NO: 993); MGA, Del, A_7, 11, high expression, KKTRPQKIPHP (SEQ ID NO: 994); BAZ2B, In, A_7, 11, high expression, KKVDRYFQSEL (SEQ ID NO: 995); PDS5B, Del, A_7, 11, high expression, KKVTRETTLIL (SEQ ID NO: 996); IFT74, In, A_7, 11, high expression, KKYRKFQKASL (SEQ ID NO: 997); UGP2, Del, A_7, 11, high expression, KKYYRSTIIVV (SEQ ID NO: 998); APPL2, Del, A_7, 11, high expression, KNKKAHAPART (SEQ ID NO: 999); ASH1L, Del, A_7, 11, high expression, KRGGILLSMFL (SEQ ID NO: 1001); GALNT1, Del, A_7, 11, high expression, KRREDFLLEMF (SEQ ID NO: 1002); CPVL, Del, A_7, 11, high expression, KSTSRSSAMNA (SEQ ID NO: 1003); CEP170, Del, A_7, 11, high expression, KTMKLEHQGAA (SEQ ID NO: 1004); NRIP1, Del, A_7, 11, high expression, KTPALREYTMM (SEQ ID NO: 1005); BRCA2, Del, A_7, 11, driver, essential, KIKKKLLQFQK (SEQ ID NO: 1006); U2SURP, Del, A_7, 11, essential, KKAIWNSSKKN (SEQ ID NO: 1008); TEX10, Del, A_7, 11, essential, KKENANMIFKK (SEQ ID NO: 1009); CHD8, In, A_7, 11, essential, KKLGTQTGNNY (SEQ ID NO: 1010); ORC6, Del, A_7, 11, essential, KKLYLIDCVNN (SEQ ID NO: 1011); TAF2, In, A_7, 11, essential, KKNPTDEWRRS (SEQ ID NO: 1012); RPAP3, Del, A_7, 11, essential, KKPERKTQKTG (SEQ ID NO: 1013); NAA35, Del, A_7, 11, essential, KKQRKKRKFAH (SEQ ID NO: 1014); BARD1, In, A_7, 11, essential, KKRCKCSARLI (SEQ ID NO: 1015); ZC3H18, In, A_7, 11, essential, KKSNYSKRTGA (SEQ ID NO: 1016); RSPO3, In, A_7, 11, driver, KKRKGEEKKKT (SEQ ID NO: 1017); SFPQ, In, A_7, 12, driver, essential, high expression, KKHERCKRQIGK (SEQ ID NO: 1018); GMPS, In, A_7, 12, driver, essential, high expression, KKKCQRRWSFQH (SEQ ID NO: 1019); MRPS31, In, A_7, 12, essential, high expression, KKKEKYIHREKT (SEQ ID NO: 1020); XRCC5, Del, A_7, 12, essential, high expression, KKKIYKKKQFIA (SEQ ID NO: 1021); TLN1, In, A_7, 12, essential, high expression, KKKQGSLWAGRR (SEQ ID NO: 1022); ESF1, In, A_7, 12, essential, high expression, KKKTEKETEGSC (SEQ ID NO: 1023); EIF3J, In, A_7, 12, essential, high expression, KKNYQFTDCALQ (SEQ ID NO: 1024); CHD1, In, A_7, 12, essential, high expression, KKQRTRRDSVFN (SEQ ID NO: 1025); RIF1, In, A_7, 12, essential, high expression, KKSFNFIKENIN (SEQ ID NO: 1026); CKAP5, Del, A_7, 12, essential, high expression, KNGKREKRPWSL (SEQ ID NO: 1027); CEP350, Del, A_7, 12, essential, high expression, KSQNNNWKKSAY (SEQ ID NO: 1028); AHCTF1, Del, A_7, 12, essential, high expression, KTHPIRTSLKMN (SEQ ID NO: 1029); CLIP1, In, A_7, 12, driver, high expression, KKKRIQVHKRRR (SEQ ID NO: 1030); KRAS, Del, A_7, 12, driver, high expression, KKSAKKKRLLAV (SEQ ID NO: 1031); PPFIBP1, Del, A_7, 12, driver, high expression, KSLNQPKMNWHL (SEQ ID NO: 1032); LRRK2, Del, A_7, 12, high expression, KIFFWLEPLMAS (SEQ ID NO: 1033); GCC2, Del, A_7, 12, high expression, KILIVCRKSFYS (SEQ ID NO: 1034); TFPI, Del, A_7, 12, high expression, KKCVQEIMQTGL (SEQ ID NO: 1035); PHF11, Del, A_7, 12, high expression, KKFMHLNKGGSS (SEQ ID NO: 1036); CCPG1, In, A_7, 12, high expression, KKGNSNLTGKTH (SEQ ID NO: 1037); CHD9, In, A_7, 12, high expression, KKIRRRYRREAI (SEQ ID NO: 1038); TRDN, Del, A_7, 12, high expression, KKLNIEKENLHL (SEQ ID NO: 1039); BRE, In, A_7, 12, high expression, KKLSVQPQMGWK (SEQ ID NO: 1040); MAP4, In, A_7, 12, high expression, KKNRGSCYNPKA (SEQ ID NO: 1042); ZNF117, In, A_7, 12, high expression, KKNSYWRETLQM (SEQ ID NO: 1043); ZNF429, In, A_7, 12, high expression, KKNSYWRGTLQI (SEQ ID NO: 1044); ZNF43, In, A_7, 12, high expression, KKNSYWRKTLQM (SEQ ID NO: 1045); ECHDC1, In, A_7, 12, high expression, KKNTSAVSWWIH (SEQ ID NO: 1046); USP24, In, A_7, 12, high expression, KKQEGWIQVISA (SEQ ID NO: 1047); NEB, In, A_7, 12, high expression, KKQRENDWSPQH (SEQ ID NO: 1048); IFI16, Del, A_7, 12, high expression, KKQTRETMTPRA (SEQ ID NO: 1049); EPB41, Del, A_7, 12, high expression, KKQWSLKHRRKQ (SEQ ID NO: 1050); MPHOSPH8, In, A_7, 12, high expression, KKRKICRIPGGI (SEQ ID NO: 1051); ATP13A3, Del, A_7, 12, high expression, KKSVFSVSVLKE (SEQ ID NO: 1052); RABGAP1, In, A_7, 12, high expression, KKTNGISLQHED (SEQ ID NO: 1053); TAOK3, Del, A_7, 12, high expression, KKYFSKRHGMDP (SEQ ID NO: 1055); XIRP2, Del, A_7, 12, high expression, KMGFFPHCPQRR (SEQ ID NO: 1056); ANK2, Del, A_7, 12, high expression, KMMRQNLQKHLS (SEQ ID NO: 1057); AOX1, Del, A_7, 12, high expression, KMSILKQCCCLI (SEQ ID NO: 1058); DAB2, Del, A_7, 12, high expression, KRKALKRQMNIS (SEQ ID NO: 1059); PPIG, Del, A_7, 12, high expression, KRLIEIKVPSQK (SEQ ID NO: 1060); ACAP2, Del, A_7, 12, high expression, KSLSLKVLKKRG (SEQ ID NO: 1061); BAGS, Del, A_7, 12, high expression, KTPASGKPGEEQ (SEQ ID NO: 1062); PIK3CA, Del, A_7, 12, driver, essential, KKLEVCCYPLNN (SEQ ID NO: 1063); REV3L, Del, A_7, 12, essential, KIYLTFPIIYLR (SEQ ID NO: 1064); POLA1, Del, A_7, 12, essential, KKAYLMYMLPSG (SEQ ID NO: 1065); BUB1, In, A_7, 12, essential, KKCKVIWGLGSQ (SEQ ID NO: 1066); KIF11, In, A_7, 12, essential, KKGTRWLSQYTV (SEQ ID NO: 1067); MCM10, In, A_7, 12, essential, KKKERTTCLSGI (SEQ ID NO: 1068); RNMT, In, A_7, 12, essential, KKNISGILRRKD (SEQ ID NO: 1069); CHEK1, In, A_7, 12, essential, KKNRFCSSSSAA (SEQ ID NO: 1070); DNAJC2, In, A_7, 12, essential, KKRRNEQNKNIS (SEQ ID NO: 1072); NCAPG, Del, A_7, 12, essential, KKSKHFIVKVQK (SEQ ID NO: 1073); XRCC2, In, A_7, 12, essential, KKTFFYYWRKWG (SEQ ID NO: 1074); NPAT, Del, A_7, 12, essential, KKVNFVKILMIQ (SEQ ID NO: 1075); ERCC6L, Del, A_7, 12, essential, KRTLSDILVNKN (SEQ ID NO: 1077); DIMT1, Del, A_7, 12, essential, KTTEFTVQSIIL (SEQ ID NO: 1078); CHD8, Del, A_7, 12, essential, KTWHPNRKQLLK (SEQ ID NO: 1079); XPO1, In, A_7, 13, driver, essential, high expression, KKIRCWPHYQDVI (SEQ ID NO: 1080); ZRANB2, Del, A_7, 13, essential, high expression, KKDEQDHGHPKAR (SEQ ID NO: 1081); DLD, Del, A_7, 13, essential, high expression, KKFQKRWLLLVQE (SEQ ID NO: 1082); ZNF131, In, A_7, 13, essential, high expression, KKKEDCRNFKCYH (SEQ ID NO: 1083); IWS1, In, A_7, 13, essential, high expression, KKKQICTRSENQH (SEQ ID NO: 1084); CKAP5, In, A_7, 13, essential, high expression, KKKTGENKEQSQM (SEQ ID NO: 1085); AKIRIN2, Del, A_7, 13, essential, high expression, KKNSPYLLYGRLG (SEQ ID NO: 1086); SNW1, In, A_7, 13, essential, high expression, KKNVECAGHSGGF (SEQ ID NO: 1087); ESF1, Del, A_7, 13, essential, high expression, KKPRLKKKSIQKI (SEQ ID NO: 1088); CEP350, In, A_7, 13, essential, high expression, KKPTGSPAEVITK (SEQ ID NO: 1089); CDC5L, In, A_7, 13, essential, high expression, KKRQTAFEKEKRI (SEQ ID NO: 1090); USP1, In, A_7, 13, essential, high expression, KKRRSKKCGRITY (SEQ ID NO: 1091); IPO7, In, A_7, 13, essential, high expression, KKRWSPAYDWLFS (SEQ ID NO: 1092); PRPF4B, Del, A_7, 13, essential, high expression, KNMVKMLVFILKL (SEQ ID NO: 1093); EPAS1, Del, A_7, 13, driver, high expression, KKAKTCPQSGTSS (SEQ ID NO: 1095); CYLD, Del, A_7, 13, driver, high expression, KMRKLAFPQFSSC (SEQ ID NO: 1096); ROCK1, In, A_7, 13, high expression, KKAERRKRSSREG (SEQ ID NO: 1097); NFATC3, Del, A_7, 13, high expression, KKASLNVLLIHQF (SEQ ID NO: 1098); MYH10, Del, A_7, 13, high expression, KKCKHIFRTWKNS (SEQ ID NO: 1099); CMYA5, In, A_7, 13, high expression, KKDRKSTSFRSNC (SEQ ID NO: 1100); FUT8, Del, A_7, 13, high expression, KKECIWPQMTLLY (SEQ ID NO: 1101); KIF3A, Del, A_7, 13, high expression, KKEGEKRKSPQTR (SEQ ID NO: 1102); ROCK2, Del, A_7, 13, high expression, KKEIKTLKYPLRK (SEQ ID NO: 1103); KNG1, Del, A_7, 13, high expression, KKFTLLSTVNHWE (SEQ ID NO: 1104); LYVE1, In, A_7, 13, high expression, KKIDLCHRSFYGN (SEQ ID NO: 1105); IFI16, In, A_7, 13, high expression, KKINQRKGWTQRE (SEQ ID NO: 1106); CHD9, In, A_7, 13, high expression, KKKARKEEECRRC (SEQ ID NO: 1107); RGS2, In, A_7, 13, high expression, KKKQTASFHQAFS (SEQ ID NO: 1108); KIF21A, Del, A_7, 13, high expression, KKLGLNMKRNSKP (SEQ ID NO: 1109); STARD7, In, A_7, 13, high expression, KKMGCPGNQAGGD (SEQ ID NO: 1110); ANK3, In, A_7, 13, high expression, KKMGYQRRKKCHN (SEQ ID NO: 1111); RYR1, In, A_7, 13, high expression, KKNAEDITKCPDL (SEQ ID NO: 1113); EIF2A, In, A_7, 13, high expression, KKNAKLVSILVRR (SEQ ID NO: 1114); TP53BP1, Del, A_7, 13, high expression, KKNAQKLWKLKPV (SEQ ID NO: 1115); HNRNPD, In, A_7, 13, high expression, KKNFCWWPFSRYT (SEQ ID NO: 1117); FAM193A, In, A_7, 13, high expression, KKNGPDLRKGKRR (SEQ ID NO: 1118); AP3B1, In, A_7, 13, high expression, KKNIPSTKACTTA (SEQ ID NO: 1119); ROCK2, In, A_7, 13, high expression, KKNQRSTDEGRRL (SEQ ID NO: 1120); POLE3, In, A_7, 13, high expression, KKNRLGRARQEQG (SEQ ID NO: 1121); NEXN, In, A_7, 13, high expression, KKNRRRASKKESN (SEQ ID NO: 1122); PPFIA1, In, A_7, 13, high expression, KKNSNRWSAGHKP (SEQ ID NO: 1123); PARP14, In, A_7, 13, high expression, KKNYGCQEWPDNE (SEQ ID NO: 1124); EIF4G3, In, A_7, 13, high expression, KKNYKNSGSKPGR (SEQ ID NO: 1125); ZNF280D, In, A_7, 13, high expression, KKRSSRKGTRTCV (SEQ ID NO: 1126); EIF4G3, In, A_7, 13, high expression, KKSESCGRKWRRS (SEQ ID NO: 1127); RPS6KA3, Del, A_7, 13, high expression, KKSQALMLGSFMP (SEQ ID NO: 1128); ZNF737, In, A_7, 13, high expression, KKTFQMYRMWQSF (SEQ ID NO: 1129); EPB41L3, In, A_7, 13, high expression, KKTGSKWCLALFI (SEQ ID NO: 1130); RBM34, In, A_7, 13, high expression, KKVGRQGKRSSEC (SEQ ID NO: 1131); RAPGEF2, Del, A_7, 13, high expression, KKVKPTLWEEGTS (SEQ ID NO: 1132); SMCHD1, Del, A_7, 13, high expression, KKWKIFRSWGIIP (SEQ ID NO: 1133); GLS, Del, A_7, 13, high expression, KNLILEEKVVIKG (SEQ ID NO: 1134); BLOC1S2, Del, A_7, 13, high expression, KNWKPSTRSWRSD (SEQ ID NO: 1135); BTN3A1, Del, A_7, 13, high expression, KRLSSERKRESKS (SEQ ID NO: 1136); ZC3H15, Del, A_7, 13, high expression, KRRRRLSKTKLSV (SEQ ID NO: 1137); ARHGAP10, Del, A_7, 13, high expression, KRSLTKRQKRIIV (SEQ ID NO: 1138); STXBP3, Del, A_7, 13, high expression, KSLKTTTRLMKRA (SEQ ID NO: 1139); ITPR2, Del, A_7, 13, high expression, KSQKNSLKFSMIE (SEQ ID NO: 1140); UBR1, Del, A_7, 13, high expression, KTPWSGTLEKEIV (SEQ ID NO: 1141); HIST1H1D, Del, A_7, 13, high expression, KTTAVSSLASRAW (SEQ ID NO: 1142); DIS3, Del, A_7, 13, essential, KKCQQTTSCKLSS (SEQ ID NO: 1143); CHD8, In, A_7, 13, essential, KKIYRGPGYKDHR (SEQ ID NO: 1144); TAF1B, In, A_7, 13, essential, KKKRNGGESTETI (SEQ ID NO: 1145); POP1, Del, A_7, 13, essential, KKLISHLMKWAHP (SEQ ID NO: 1146); REV1, Del, A_7, 13, essential, KKPLVHQKGFRVL (SEQ ID NO: 1147); YEATS4, Del, A_7, 13, essential, KKQALRLQSLRRD (SEQ ID NO: 1148); TRMT6, In, A_7, 13, essential, KKSNFRKTVVLPG (SEQ ID NO: 1149); ATAD5, Del, A_7, 13, essential, KNLTHPLKMYLEK (SEQ ID NO: 1150); KIAA1524, Del, A_7, 13, essential, KNVKGLPRPLKFC (SEQ ID NO: 1151); KMTSB, Del, A_7, 13, essential, KTMQLLTENLQLA (SEQ ID NO: 1152); TSC1, In, A_7, 13, driver, KKQKPCSPADSEA (SEQ ID NO: 1153); BTK, In, A_7, 13, driver, KKSSSRKTDSEKR (SEQ ID NO: 1154); TET1, Del, A_7, 13, driver, KTLKITYRVWLHD (SEQ ID NO: 1155); SMARCB1, Del, A_7, 14, driver, essential, high expression, KKQNLTLRITDTRL (SEQ ID NO: 1156); SPEN, Del, A_7, 14, driver, essential, high expression, KTPNQREEDLETPG (SEQ ID NO: 1157); NAE1, In, A_7, 14, essential, high expression, KKGPQSYSMDCDHS (SEQ ID NO: 1158); GNL3, In, A_7, 14, essential, high expression, KKGSRTSSKIKKGG (SEQ ID NO: 1159); CCT3, In, A_7, 14, essential, high expression, KKICKSGKDTWRHH (SEQ ID NO: 1160); DYNC1I2, Del, A_7, 14, essential, high expression, KKLPQSMCFTASQL (SEQ ID NO: 1162); CCT4, Del, A_7, 14, essential, high expression, KKQDVMSFSYRNLF (SEQ ID NO: 1163); XRCC5, In, A_7, 14, essential, high expression, KKRRYTKRNSLLLK (SEQ ID NO: 1164); DDX47, In, A_7, 14, essential, high expression, KKTTYNNSNSWSTD (SEQ ID NO: 1165); PCF11, Del, A_7, 14, essential, high expression, KNIFRIRPMAKMMM (SEQ ID NO: 1166); IDH1, In, A_7, 14, driver, high expression, KKNQWRFCGRDARR (SEQ ID NO: 1167); MAP4K3, Del, A_7, 14, high expression, KKDLLLKNYYSILL (SEQ ID NO: 1168); DST, In, A_7, 14, high expression, KKEHSQRLYLQTRF (SEQ ID NO: 1169); VPS13C, In, A_7, 14, high expression, KKFNSAKSDCILQR (SEQ ID NO: 1170); DST, Del, A_7, 14, high expression, KKFPLYSLLLVQRI (SEQ ID NO: 1171); PHF3, Del, A_7, 14, high expression, KKHLGRLYLLLKQE (SEQ ID NO: 1172); ALMS1, In, A_7, 14, high expression, KKKQGAFSSSSWEV (SEQ ID NO: 1173); GOLGB1, Del, A_7, 14, high expression, KKLCLHSKMTETDY (SEQ ID NO: 1174); RUFY3, Del, A_7, 14, high expression, KKLFSDKINPSGGL (SEQ ID NO: 1175); FAM98A, Del, A_7, 14, high expression, KKLKKEAVVRSFKS (SEQ ID NO: 1176); LIMCH1, In, A_7, 14, high expression, KKNGEVTGWRRWDK (SEQ ID NO: 1177); ANGPTL1, In, A_7, 14, high expression, KKNKPEKIPSCHRW (SEQ ID NO: 1178); TAOK3, In, A_7, 14, high expression, KKNTFPRDTEWTLE (SEQ ID NO: 1179); HP1BP3, Del, A_7, 14, high expression, KKQFLPGLPFLPAS (SEQ ID NO: 1180); APOB, In, A_7, 14, high expression, KKQKQCIRFCHQIL (SEQ ID NO: 1181); SOS2, In, A_7, 14, high expression, KKQLDGSPYFSSLS (SEQ ID NO: 1182); PARP14, Del, A_7, 14, high expression, KKQLMSKLIIGTTQ (SEQ ID NO: 1183); OSBPL1A, In, A_7, 14, high expression, KKQNLVRSTGDAGH (SEQ ID NO: 1184); ROCK1, In, A_7, 14, high expression, KKQRKFKENTGTTK (SEQ ID NO: 1185); NEXN, In, A_7, 14, high expression, KKRITFSRKIETNF (SEQ ID NO: 1186); OCIAD1, In, A_7, 14, high expression, KKRSQSKQVWRYLG (SEQ ID NO: 1187); BLOC1S2, In, A_7, 14, high expression, KKTGSQVQEAGEAM (SEQ ID NO: 1188); TNKS2, In, A_7, 14, high expression, KKTVYCSECQLQRH (SEQ ID NO: 1189); UHRF1BP1L, In, A_7, 14, high expression, KKWISAVPHRKLQH (SEQ ID NO: 1190); ZNF638, Del, A_7, 14, high expression, KMKLFRKYCHQLVL (SEQ ID NO: 1193); AKAP12, Del, A_7, 14, high expression, KNQSPSWKRKAKTP (SEQ ID NO: 1195); EPB41L3, Del, A_7, 14, high expression, KNRFEVVLGTFHLM (SEQ ID NO: 1196); PNRC1, Del, A_7, 14, high expression, KSLLKIPRTLKIRI (SEQ ID NO: 1197); RBL2, Del, A_7, 14, high expression, KSSLREKKKISLGF (SEQ ID NO: 1198); FNIP2, Del, A_7, 14, high expression, KTSSASAFSRSLHF (SEQ ID NO: 1199); MYB, In, A_7, 14, driver, essential, KKTGPGQPLSHWRL (SEQ ID NO: 1200); RNPC3, Del, A_7, 14, essential, KIMIYLLLKLMHPI (SEQ ID NO: 1201); RIOK1, Del, A_7, 14, essential, KKERRRQPRRKKAN (SEQ ID NO: 1202); TOP2A, Del, A_7, 14, essential, KKGLPQKELKGIQL (SEQ ID NO: 1203); CSTF3, In, A_7, 14, essential, KKIWRHSRVCPGLY (SEQ ID NO: 1204); C12orf65, In, A_7, 14, essential, KKKSKGNPGKKEAT (SEQ ID NO: 1205); RPF1, Del, A_7, 14, essential, KKLFHSASQEISQT (SEQ ID NO: 1206); XPOS, In, A_7, 14, essential, KKNKANAGDGGAGQ (SEQ ID NO: 1208); SMC2, In, A_7, 14, essential, KKNSCTENYRKKGG (SEQ ID NO: 1209); MASTL, In, A_7, 14, essential, KKSRPCKHPFSLSV (SEQ ID NO: 1210); RBM48, In, A_7, 14, essential, KKTTNAEGICSKNY (SEQ ID NO: 1211); TET1, Del, A_7, 14, driver, KKQNLNHPCQSEAF (SEQ ID NO: 1212); SCFD1, Del, A_7, 15, essential, high expression, KKDLLISIQMLPLLF (SEQ ID NO: 1213); MRPSS, Del, A_7, 15, essential, high expression, KKEEAKELKRRKERI (SEQ ID NO: 1214); EIF2S2, Del, A_7, 15, essential, high expression, KKLKRYLILMKLKKV (SEQ ID NO: 1215); CCT6A, In, A_7, 15, essential, high expression, KKNNRTEKESLWRFR (SEQ ID NO: 1216); EIF5B, In, A_7, 15, essential, high expression, KKRTRSLCKNRTYPW (SEQ ID NO: 1217); CCT5, In, A_7, 15, essential, high expression, KKSGRCEDCNSHMSI (SEQ ID NO: 1218); PCNT, Del, A_7, 15, essential, high expression, KKWQQSCSSSLWTSC (SEQ ID NO: 1219); SDAD1, Del, A_7, 15, essential, high expression, KRRKNQRCLTFQPFT (SEQ ID NO: 1220); CYLD, In, A_7, 15, driver, high expression, KKNFSFSGIKYNRFT (SEQ ID NO: 1221); KRAS, In, A_7, 15, driver, high expression, KKNQQRRKDSWLCEN (SEQ ID NO: 1222); PCM1, Del, A_7, 15, driver, high expression, KKSLPEILSRSLWKR (SEQ ID NO: 1223); VPS41, Del, A_7, 15, high expression, KILKDIRFWILAWHI (SEQ ID NO: 1224); FTH1, In, A_7, 15, high expression, KKCESVTTGTAQTGH (SEQ ID NO: 1225); AKAP12, Del, A_7, 15, high expression, KKEKVSLPGHHSKRW (SEQ ID NO: 1226); ZNF292, Del, A_7, 15, high expression, KKGKLKKLNQHQQLS (SEQ ID NO: 1227); CLCN3, Del, A_7, 15, high expression, KKGRCYQLPQLQGFL (SEQ ID NO: 1228); TM9SF2, In, A_7, 15, high expression, KKKHVIELSTSLDCG (SEQ ID NO: 1229); KIF3A, In, A_7, 15, high expression, KKKKGKKESLPRQDD (SEQ ID NO: 1230); CPNE3, In, A_7, 15, high expression, KKKLQEFRCYQCETV (SEQ ID NO: 1231); PARP1, In, A_7, 15, high expression, KKKQGPGQGGRYQQI (SEQ ID NO: 1232); ZNF124, Del, A_7, 15, high expression, KKLILEKSPINVKKC (SEQ ID NO: 1233); PDS5B, In, A_7, 15, high expression, KKNARHRTGGEIRYG (SEQ ID NO: 1234); APOB, In, A_7, 15, high expression, KKNDFQFPCGSLRLS (SEQ ID NO: 1235); KBTBD2, In, A_7, 15, high expression, KKNKCRELCTIVEFC (SEQ ID NO: 1236); G3BP2, In, A_7, 15, high expression, KKNKSCKRARNQRWW (SEQ ID NO: 1237); ANK3, In, A_7, 15, high expression, KKNTGEQATRLPSFC (SEQ ID NO: 1238); MAP2, Del, A_7, 15, high expression, KKQKFRPTLPPGNSF (SEQ ID NO: 1239); OGFRL1, Del, A_7, 15, high expression, KKQKKQLEDSSWLIK (SEQ ID NO: 1240); HIBADH, In, A_7, 15, high expression, KKSEEGLIINRFQHY (SEQ ID NO: 1241); LARP4, In, A_7, 15, high expression, KKSENSLQRSKTTRR (SEQ ID NO: 1242); SNX25, Del, A_7, 15, high expression, KKSSCLLLASCLSNL (SEQ ID NO: 1243); USP33, In, A_7, 15, high expression, KKTAQEIQKCYFRHI (SEQ ID NO: 1244); SYNE2, In, A_7, 15, high expression, KKVLFGCPVNKTGSG (SEQ ID NO: 1245); MRPL1, Del, A_7, 15, high expression, KKVLKKKHQMRKKMK (SEQ ID NO: 1246); DENNDSA, In, A_7, 15, high expression, KKVTFQTPEAAPLRP (SEQ ID NO: 1247); XIRP2, In, A_7, 15, high expression, KKWVSSLTVHREDKG (SEQ ID NO: 1248); ADNP, Del, A_7, 15, high expression, KKYLVTLGWNTVKNI (SEQ ID NO: 1249); ANK3, Del, A_7, 15, high expression, KNGLPETEKMPQLMP (SEQ ID NO: 1250); DST, Del, A_7, 15, high expression, KRAQPKTVPSNQILR (SEQ ID NO: 1251); LMOD2, Del, A_7, 15, high expression, KRKKGKRSRNSQTVF (SEQ ID NO: 1252); PHF20L1, Del, A_7, 15, high expression, KRKTTMKQLQCWSRY (SEQ ID NO: 1253); MAN1A2, Del, A_7, 15, high expression, KRWGIWPALLGECLH (SEQ ID NO: 1254); SAFB2, Del, A_7, 15, high expression, KSLKTLRRKKKTRMS (SEQ ID NO: 1255); ACAP2, Del, A_7, 15, high expression, KTFLKWLRLWLMVQT (SEQ ID NO: 1256); MIA3, Del, A_7, 15, high expression, KTLNYLRKGNLNLNQ (SEQ ID NO: 1257); STIL, In, A_7, 15, driver, essential, KKNTSFKTGGQRLCP (SEQ ID NO: 1258); BRCA2, Del, A_7, 15, driver, essential, KNGLEKEYLMVNQKE (SEQ ID NO: 1259); MDC1, In, A_7, 15, essential, KKIKDHIFLCDSSRE (SEQ ID NO: 1260); RIOK1, In, A_7, 15, essential, KKKKGEDSQDEKRQI (SEQ ID NO: 1261); MCM6, In, A_7, 15, essential, KKKNHRESYSSTHTL (SEQ ID NO: 1262); ATAD5, In, A_7, 15, essential, KKMFCPCFSLFKFSL (SEQ ID NO: 1263); REV1, In, A_7, 15, essential, KKNHWFTKKDSESFE (SEQ ID NO: 1264); RPAP3, In, A_7, 15, essential, KKNQRGKHKKQDKIL (SEQ ID NO: 1265); LIPT1, In, A_7, 15, essential, KKNSKKWAHFTVNFQ (SEQ ID NO: 1266); TAF1, Del, A_7, 15, essential, KKPFLPIQKAASGRG (SEQ ID NO: 1267); XRCC2, Del, A_7, 15, essential, KNIFLLLEKVGLNFV (SEQ ID NO: 1268); DDX10, In, A_7, 16, driver, essential, high expression, KKNIERFARSSVPFGD (SEQ ID NO: 1269); SETD2, Del, A_7, 16, driver, essential, high expression, KMISKIEGLLKKGGRK (SEQ ID NO: 1270); ZC3H8, Del, A_7, 16, essential, high expression, KIKILKLVTRMANRRK (SEQ ID NO: 1271); RARS2, In, A_7, 16, essential, high expression, KKAFSASIPNAEDHGI (SEQ ID NO: 1272); MRPS31, Del, A_7, 16, essential, high expression, KKGKIYSQGKDLIFLT (SEQ ID NO: 1273); PRPF40A, In, A_7, 16, essential, high expression, KKKTAIRVSFCFRTFF (SEQ ID NO: 1274); ZRANB2, In, A_7, 16, essential, high expression, KKKTNKITVTRKPGDW (SEQ ID NO: 1275); RPL12, Del, A_7, 16, essential, high expression, KKLVMTLPRQRVTGRA (SEQ ID NO: 1276); GON4L, In, A_7, 16, essential, high expression, KKNDTQAWASYPRRQM (SEQ ID NO: 1277); CWF19L2, In, A_7, 16, essential, high expression, KKSKERKEKKEQETEI (SEQ ID NO: 1279); MRPL33, In, A_7, 16, essential, high expression, KKYAPFKRWIENDFDL (SEQ ID NO: 1280); KTN1, In, A_7, 16, driver, high expression, KKGKRMYGWNFRVRGG (SEQ ID NO: 1281); MIA3, Del, A_7, 16, high expression, KIRKFWMTQLKIFVLC (SEQ ID NO: 1282); USP34, Del, A_7, 16, high expression, KIWLILMVKNLDVKRS (SEQ ID NO: 1283); ZC3H15, In, A_7, 16, high expression, KKEGEDYRRQNFRFEE (SEQ ID NO: 1284); HMOX2, In, A_7, 16, high expression, KKELWGPRKGEPNENG (SEQ ID NO: 1285); HNRNPD, Del, A_7, 16, high expression, KKFLLVAFLQIHLKRK (SEQ ID NO: 1286); CEP170, In, A_7, 16, high expression, KKFRTFYKQRRQSGTK (SEQ ID NO: 1287); CPNE3, Del, A_7, 16, high expression, KKGKRKKATRIQVLSV (SEQ ID NO: 1288); C1S, In, A_7, 16, high expression, KKGLETSLSWRSNALP (SEQ ID NO: 1289); IFI16, In, A_7, 16, high expression, KKIQEHCSTKRIRGHQ (SEQ ID NO: 1290); LYN, Del, A_7, 16, high expression, KKKASSPATMWPNSTP (SEQ ID NO: 1291); ANK3, In, A_7, 16, high expression, KKKETPQTVPGSEEKV (SEQ ID NO: 1292); RNF19A, In, A_7, 16, high expression, KKKKNFNRLPVSEEKR (SEQ ID NO: 1293); AIDA, In, A_7, 16, high expression, KKKVYQHQVFCFHGDG (SEQ ID NO: 1294); CCDC66, Del, A_7, 16, high expression, KKLKWFHLSQLKINLS (SEQ ID NO: 1295); LRRFIP2, In, A_7, 16, high expression, KKNWGPRETERIHCLP (SEQ ID NO: 1296); ZAK, In, A_7, 16, high expression, KKREINHSKQLPQKFC (SEQ ID NO: 1297); PHF3, In, A_7, 16, high expression, KKSKSCCRSSSQTFRQ (SEQ ID NO: 1298); DNAJC8, In, A_7, 16, high expression, KKTIKEGRKTYNCRGG (SEQ ID NO: 1299); RAI14, In, A_7, 16, high expression, KKTQSSTTSYQSYPVE (SEQ ID NO: 1301); FER, In, A_7, 16, high expression, KKTRRLFGARESWETW (SEQ ID NO: 1302); DDX3Y, In, A_7, 16, high expression, KKYEYYKGFVGSSCRS (SEQ ID NO: 1303); TDO2, In, A_7, 16, high expression, KKYHQRPGRGIHKDSG (SEQ ID NO: 1304); PCOLCE2, Del, A_7, 16, high expression, KNCLQLQNSLSPPHSL (SEQ ID NO: 1305); ZCRB1, Del, A_7, 16, high expression, KRKKRKLLNQKKKLRK (SEQ ID NO: 1306); GOLGA4, Del, A_7, 16, high expression, KRWKKLSRKQRRCKKR (SEQ ID NO: 1307); SOS2, Del, A_7, 16, high expression, KTTGWQPLFLFIIVVL (SEQ ID NO: 1308); PPM1D, In, A_7, 16, driver, essential, KKFEDVNSWPNESPRN (SEQ ID NO: 1309); SUZ12, In, A_7, 16, driver, essential, KKKKGCKLSNKASSHR (SEQ ID NO: 1310); CREBBP, Del, A_7, 16, driver, essential, KKSLNQRSYARPSCQP (SEQ ID NO: 1311); U2SURP, Del, A_7, 16, essential, KILQISLPMKDHHLFL (SEQ ID NO: 1312); ASCC3, In, A_7, 16, essential, KKGWRRFRSFRRTYVL (SEQ ID NO: 1313); NHLRC2, In, A_7, 16, essential, KKKEFTLCCRLLQSQD (SEQ ID NO: 1314); CENPC, In, A_7, 16, essential, KKKFFNIVFRNSKTKK (SEQ ID NO: 1315); CMTR1, In, A_7, 16, essential, KKKSCRACPEPQLHVR (SEQ ID NO: 1316); CENPE, Del, A_7, 16, essential, KKMSMRHRKKCLKWSN (SEQ ID NO: 1318); PPAT, In, A_7, 16, essential, KKNIRNRRMGGVFRIL (SEQ ID NO: 1319); DIS3, In, A_7, 16, essential, KKNVSRQPAASYLHNK (SEQ ID NO: 1320); RPP40, Del, A_7, 16, essential, KMNMVFEKEENIYITL (SEQ ID NO: 1321); C12orf65, Del, A_7, 16, essential, KRSYLKNCGSQVKRST (SEQ ID NO: 1322); TOP3A, Del, A_7, 16, essential, KTTRPRGSPTCCQTVA (SEQ ID NO: 1323); CNTRL, In, A_7, 16, driver, KKVGTNQKGFSSSRRK (SEQ ID NO: 1324); STATSB, Del, A_7, 16, driver, KNISSLIGMMGPFWGL (SEQ ID NO: 1325); DDX10, In, A_7, 17, driver, essential, high expression, KKNDQSCRSKKSNEEKF (SEQ ID NO: 1326); XPO1, Del, A_7, 17, driver, essential, high expression, KNTLLASLSRRHLTQLV (SEQ ID NO: 1327); PCF11, Del, A_7, 17, essential, high expression, KIFRVLNLETLAIYLLQ (SEQ ID NO: 1328); TRPM7, Del, A_7, 17, essential, high expression, KKHLTLARMKHFIYFKH (SEQ ID NO: 1329); PRPF40A, Del, A_7, 17, essential, high expression, KKKRNKQSSCEREIGKP (SEQ ID NO: 1330); ZNF131, In, A_7, 17, essential, high expression, KKRKEEALRMPGLQQCV (SEQ ID NO: 1331); DIDO1, Del, A_7, 17, essential, high expression, KMSRPLRDFTSRARPRR (SEQ ID NO: 1332); TTC1, Del, A_7, 17, essential, high expression, KTCRMKRNRKEEKRALD (SEQ ID NO: 1333); IL6ST, In, A_7, 17, driver, high expression, KKSREKRSCLRVGPTSC (SEQ ID NO: 1334); TRIP11, Del, A_7, 17, driver, high expression, KREFINLKMIKWTLLKS (SEQ ID NO: 1335); SLTM, Del, A_7, 17, high expression, KKEIKTTEGKRSCLLKR (SEQ ID NO: 1336); TM9SF2, In, A_7, 17, high expression, KKERRVQGRNRTICEQT (SEQ ID NO: 1337); ZFAND6, In, A_7, 17, high expression, KKESLFHVQEESGTYWV (SEQ ID NO: 1338); NUFIP2, Del, A_7, 17, high expression, KKGKLGAIVPRVVKTLI (SEQ ID NO: 1339); ALAS1, Del, A_7, 17, high expression, KKLMRKRMTTPIEFLKL (SEQ ID NO: 1340); SETX, Del, A_7, 17, high expression, KKLSLNSKKNVKTRRIL (SEQ ID NO: 1341); ERGIC2, In, A_7, 17, high expression, KKNFKFGKRVGCLSEGS (SEQ ID NO: 1342); FNIP2, In, A_7, 17, high expression, KKNGGTGEGLWPLLGGQ (SEQ ID NO: 1343); USP47, In, A_7, 17, high expression, KKNMEESWHCLFGLSYL (SEQ ID NO: 1344); PHF11, In, A_7, 17, high expression, KKNSCISTKVAAVEGRD (SEQ ID NO: 1345); EPB41L5, Del, A_7, 17, high expression, KKPKDKSCLIRLCTTWT (SEQ ID NO: 1346); SECISBP2L, In, A_7, 17, high expression, KKRKREGNCKTKTTHST (SEQ ID NO: 1347); BAZ1B, In, A_7, 17, high expression, KKRRKEMGSSKISASQI (SEQ ID NO: 1348); EEA1, In, A_7, 17, high expression, KKSDLDRKLAEKRTRLY (SEQ ID NO: 1349); XIRP2, In, A_7, 17, high expression, KKSGTINDWSKRREINN (SEQ ID NO: 1350); ZNF638, In, A_7, 17, high expression, KKSIRRCSTTIWAWDRI (SEQ ID NO: 1351); CYP8B1, In, A_7, 17, high expression, KKTGAEGIWIPFSARGP (SEQ ID NO: 1352); MED13, Del, A_7, 17, high expression, KKVLLENLVYRNFGSGA (SEQ ID NO: 1353); TTC37, In, A_7, 17, high expression, KKYCTKDHSEGSSPFSR (SEQ ID NO: 1354); PALLD, Del, A_7, 17, high expression, KKYGPQPVAMQHFRTRD (SEQ ID NO: 1355); TTN, Del, A_7, 17, high expression, KRFLQKSLRWKNLSQPK (SEQ ID NO: 1356); CWC25, In, A_7, 17, essential, KKGEEEKEGEEKEAQET (SEQ ID NO: 1358); C11orf57, Del, A_7, 17, essential, KKGHTKNRRKAKRKPQI (SEQ ID NO: 1359); ORC3, Del, A_7, 17, essential, KKGLLLANGSLLLLSLS (SEQ ID NO: 1360); ANLN, Del, A_7, 17, essential, KKHPSPRLLLQSDSSHL (SEQ ID NO: 1361); SUGT1, In, A_7, 17, essential, KKLCCCPRNFYRRTKIR (SEQ ID NO: 1362); NIFK, In, A_7, 17, essential, KKTRTTYSWSSLCAPPT (SEQ ID NO: 1363); TOPBP1, In, A_7, 17, essential, KKTVHISVIISESSRTY (SEQ ID NO: 1364); HSPA14, Del, A_7, 17, essential, KMLLEKQLELLDLMFCD (SEQ ID NO: 1365); PPAT, Del, A_7, 17, essential, KNLEYCQTTLKAKELFL (SEQ ID NO: 1366); ORCS, Del, A_7, 17, essential, KNTKRQAIISLGQNHFH (SEQ ID NO: 1367); NMT1, Del, A_7, 17, essential, KRRKKKAVRQIQPRISL (SEQ ID NO: 1368); MSH2, Del, A_7, 17, driver, KKLTFPQKTFIRTSTGC (SEQ ID NO: 1369); NUP98, In, A_7, 18, driver, essential, high expression, KKQPCGGCIQLPHRQKDQ (SEQ ID NO: 1370); ATRX, Del, A_7, 18, driver, essential, high expression, KRQSLKSIKKSKAETEER (SEQ ID NO: 1371); IWS1, In, A_7, 18, essential, high expression, KKASTEKINFTACCSYAP (SEQ ID NO: 1372); FNBP4, Del, A_7, 18, essential, high expression, KKEGKTRQRRVRPKCHLW (SEQ ID NO: 1373); FERMT2, Del, A_7, 18, essential, high expression, KKIGLTMLSGGKRRELGF (SEQ ID NO: 1374); VEZT, In, A_7, 18, essential, high expression, KKIQRSFGRYGHKQPSFY (SEQ ID NO: 1375); EPRS, Del, A_7, 18, essential, high expression, KKKKILLIGILRSSQSQK (SEQ ID NO: 1376); NIPBL, In, A_7, 18, essential, high expression, KKKKTRFLPTGGWGCYRR (SEQ ID NO: 1377); CHD1, In, A_7, 18, essential, high expression, KKRSSFWCGKFKEEKSKS (SEQ ID NO: 1378); CWF19L2, In, A_7, 18, essential, high expression, KKRTSTGYKVYICWQSRA (SEQ ID NO: 1379); USP9X, In, A_7, 18, essential, high expression, KKSHQCAADRNGREQRAR (SEQ ID NO: 1380); TFAM, Del, A_7, 18, essential, high expression, KKYIKMLIGRSGRYIKKR (SEQ ID NO: 1381); RIF1, Del, A_7, 18, essential, high expression, KRNDVRKKKNLFRRVHCI (SEQ ID NO: 1382); RBM39, Del, A_7, 18, essential, high expression, KTELQQWQTIYKREVLDL (SEQ ID NO: 1383); HELZ, Del, A_7, 18, high expression, KIAQLFIIMQRCLKWWNV (SEQ ID NO: 1385); ZC3H14, Del, A_7, 18, high expression, KIKELNRGNYYPDCKSTQ (SEQ ID NO: 1386); PARP1, Del, A_7, 18, high expression, KKARARSRRKVSTNLKRE (SEQ ID NO: 1388); ZFX, Del, A_7, 18, high expression, KKCTSVDIVTLRLQIHLF (SEQ ID NO: 1389); BAZ1B, Del, A_7, 18, high expression, KKEKGNGLLQNFCLTNMM (SEQ ID NO: 1390); SPAG9, In, A_7, 18, high expression, KKGLTDPSGIFRISNKTT (SEQ ID NO: 1391); NUB1, Del, A_7, 18, high expression, KKIGKTCWRPDCTSLAEN (SEQ ID NO: 1392); WASL, In, A_7, 18, high expression, KKISKSSYRPFGPSTKEI (SEQ ID NO: 1393); CLCN3, Del, A_7, 18, high expression, KKISSGIWPRRQTKTPLQ (SEQ ID NO: 1394); UPF3A, Del, A_7, 18, high expression, KKMPRLEASKMIQNIRSF (SEQ ID NO: 1395); PABPC3, In, A_7, 18, high expression, KKNERNAPKWSQSICWTI (SEQ ID NO: 1396); NR3C1, In, A_7, 18, high expression, KKNKRNSAGHYRSLTRNL (SEQ ID NO: 1397); RGS2, In, A_7, 18, high expression, KKNQITPKAVLKSKENIY (SEQ ID NO: 1398); EHBP1, Del, A_7, 18, high expression, KKQVYLLLLNLDTHIVEI (SEQ ID NO: 1400); WASL, Del, A_7, 18, high expression, KKWSRTVGQCPALDEMHC (SEQ ID NO: 1401); ZNF564, Del, A_7, 18, high expression, KNGKTRALKIGTKIRGEF (SEQ ID NO: 1402); PTTG1IP, Del, A_7, 18, high expression, KNMACLKKKTRMLDLKTT (SEQ ID NO: 1403); RSF1, Del, A_7, 18, high expression, KNPKQTCLKGGQQEQGNV (SEQ ID NO: 1404); ODF2, Del, A_7, 18, high expression, KNSRNHQRPAARNLTSEE (SEQ ID NO: 1405); OSBPL1A, Del, A_7, 18, high expression, KTKSCQKHWRRWPLNIMN (SEQ ID NO: 1406); BRCA2, Del, A_7, 18, driver, essential, KKFSMKQTLMNVKNLKTK (SEQ ID NO: 1407); BRCA2, In, A_7, 18, driver, essential, KKSRRNYFNFKNNCQSRL (SEQ ID NO: 1408); SKA3, In, A_7, 18, essential, KKFNGYYENKRVFPEVWI (SEQ ID NO: 1409); SNAPC1, Del, A_7, 18, essential, KKFTELKLQKNLRTQVIV (SEQ ID NO: 1410); MCM10, Del, A_7, 18, essential, KKGENNLPIWNLRNFRKS (SEQ ID NO: 1411); POLR3B, In, A_7, 18, essential, KKKQNQYGCETRTILFEA (SEQ ID NO: 1412); HEATR1, Del, A_7, 18, essential, KKLTSRLRRTGRITWVRL (SEQ ID NO: 1413); RPF1, In, A_7, 18, essential, KKNYSTVHRKRFHRPDCY (SEQ ID NO: 1414); GTF3C4, Del, A_7, 18, essential, KRLKAVESPIFGVLSFSS (SEQ ID NO: 1415); TTK, Del, A_7, 18, essential, KSSCFQRRKRRIYQHLRY (SEQ ID NO: 1416); EML4, In, A_7, 18, driver, KKERKTTRTERKKRGISF (SEQ ID NO: 1417); RANBP17, In, A_7, 18, driver, KKTCEDRCCEIHAKKPHE (SEQ ID NO: 1418); SETD2, In, A_7, 19, driver, essential, high expression, KKKFRVFKACRERNNSRSR (SEQ ID NO: 1419); CTCF, Del, A_7, 19, driver, essential, high expression, KKQRKPKRANCVIQRRAKM (SEQ ID NO: 1420); USP8, Del, A_7, 19, driver, essential, high expression, KTNKKKNFGKGSKRNRKRN (SEQ ID NO: 1421); CFDP1, Del, A_7, 19, essential, high expression, KKERPRAFQPGREDKVASH (SEQ ID NO: 1422); RSRC2, In, A_7, 19, essential, high expression, KKKENALAGQERRGQIPIC (SEQ ID NO: 1423); TBCE, Del, A_7, 19, essential, high expression, KTDSAKNSSQPIPDTSSSA (SEQ ID NO: 1424); PPFIBP1, In, A_7, 19, driver, high expression, KKHRSTKNEKSCGVLDGSK (SEQ ID NO: 1425); AKAP9, Del, A_7, 19, driver, high expression, KTLKLTIKSYKRSMLAFSK (SEQ ID NO: 1426); PPIG, Del, A_7, 19, high expression, KIKNLIMNQALEQMKTKAD (SEQ ID NO: 1427); PDCD10, Del, A_7, 19, high expression, KKAWKLTSRSPFFVWQLMM (SEQ ID NO: 1428); GALNT1, In, A_7, 19, high expression, KKGERTSCWRCSRASTKAS (SEQ ID NO: 1429); CLCN3, In, A_7, 19, high expression, KKKISSGIWPRRQTKTPLQ (SEQ ID NO: 1430); ZBTB38, In, A_7, 19, high expression, KKNEESQLEEGARKQEPEP (SEQ ID NO: 1431); EFCAB2, Del, A_7, 19, high expression, KKSKRHLKSLTMSRIIQWM (SEQ ID NO: 1432); APPL2, In, A_7, 19, high expression, KKTRKLMPQPEPEKFRDGK (SEQ ID NO: 1433); LONP2, Del, A_7, 19, high expression, KKYEHLVCQSRPIKSVSKR (SEQ ID NO: 1434); ZNF415, Del, A_7, 19, high expression, KKYTTLTVSGEMMKEIATK (SEQ ID NO: 1435); PHF14, Del, A_7, 19, high expression, KNGTFDETDHFWILCPWKS (SEQ ID NO: 1436); PHF3, Del, A_7, 19, high expression, KNKLIRSQRFRVAILGLNL (SEQ ID NO: 1437); KMT2A, Del, A_7, 19, driver, essential, KKLKGHLLLRFSKPQRLKN (SEQ ID NO: 1438); BLM, In, A_7, 19, driver, essential, KKRPNHKTSICHSRKDLCK (SEQ ID NO: 1439); U2SURP, In, A_7, 19, essential, KKKQFGTLQRRIKANSRGT (SEQ ID NO: 1440); TAF1B, In, A_7, 19, essential, KKNSRSWNIFRFASFSRHN (SEQ ID NO: 1441); ZC3H18, Del, A_7, 19, essential, KKQLFEKNRSLILKRKGLR (SEQ ID NO: 1442); MCM4, Del, A_7, 19, essential, KKQPLKTSSCLILYYQGLI (SEQ ID NO: 1443); SPRTN, In, A_7, 19, essential, KKRQRKGKTRKGTSIGRRE (SEQ ID NO: 1444); TET1, Del, A_7, 19, driver, KKERVSLSLFPVLERRGLR (SEQ ID NO: 1445); NBN, Del, A_7, 19, driver, KKGKGMKKIKKCLHANQQE (SEQ ID NO: 1446); SETD2, Del, A_7, 20, driver, essential, high expression, KKKIPILGRMKKFQIVLRLV (SEQ ID NO: 1447); ATRX, In, A_7, 20, driver, essential, high expression, KKNKEISRFRMHSCPLYGPW (SEQ ID NO: 1448); XRN1, Del, A_7, 20, essential, high expression, KKHNGYVLQKKLHFTFYTCL (SEQ ID NO: 1449); DHX36, In, A_7, 20, essential, high expression, KKKKNGKSLHKNRWPGCCSS (SEQ ID NO: 1450); METAP2, In, A_7, 20, essential, high expression, KKKTKEEEEQRAFCSRGTGT (SEQ ID NO: 1451); VEZT, Del, A_7, 20, essential, high expression, KNTAFIWKIWPQTAELLLTS (SEQ ID NO: 1452); FAT1, Del, A_7, 20, driver, high expression, KILMWRREQRSGCRCWIQMT (SEQ ID NO: 1453); AKAP9, In, A_7, 20, driver, high expression, KKCFRKRKEAARTTEAIGGQ (SEQ ID NO: 1454); ITPR2, Del, A_7, 20, high expression, KIQFNLTMKEAGRFYGSSFI (SEQ ID NO: 1455); BRWD1, Del, A_7, 20, high expression, KKASELRTMLKATGRNSVRN (SEQ ID NO: 1457); PARP14, In, A_7, 20, high expression, KKESFGFGKENRISNFSGVW (SEQ ID NO: 1458); ZNF195, In, A_7, 20, high expression, KKIPKMWRMWQNLYPVLTLY (SEQ ID NO: 1459); CPNE3, In, A_7, 20, high expression, KKKAKEKKLQEFRCYQCETV (SEQ ID NO: 1460); MED13, In, A_7, 20, high expression, KKKNSKTLGCSGSSHLATIS (SEQ ID NO: 1461); SKIL, In, A_7, 20, high expression, KKNDNGHSCKWKNDKQGANS (SEQ ID NO: 1462); RNF13, In, A_7, 20, high expression, KKNLSSVQAKSCSFSRRFRL (SEQ ID NO: 1463); ASH1L, In, A_7, 20, high expression, KKNYQEKQWTINENNYPQNK (SEQ ID NO: 1464); FNIP2, In, A_7, 20, high expression, KKPALPALSQGVYTSDRTDK (SEQ ID NO: 1465); KIF21A, In, A_7, 20, high expression, KKRFREVEKKRKEEEKKCGW (SEQ ID NO: 1467); CMYA5, In, A_7, 20, high expression, KKRNLRRFRGNEHKLSSYFC (SEQ ID NO: 1468); KLHDC2, Del, A_7, 20, high expression, KKSTLKVMFLLLCQEAVLCV (SEQ ID NO: 1469); ZNF280D, Del, A_7, 20, high expression, KMDVMQIHLKAHQQQKVKKA (SEQ ID NO: 1470); APPL1, Del, A_7, 20, high expression, KNLQFCKQRVKKIMKSGSVQ (SEQ ID NO: 1472); BRCA2, Del, A_7, 20, driver, essential, KKQDLPLSSICQTNVTIYWQ (SEQ ID NO: 1473); RNMT, Del, A_7, 20, essential, KKHFWNSTKKRLRTMKIKCS (SEQ ID NO: 1474); TOP2A, Del, A_7, 20, essential, KKHKWLKFCLLRVVKESFHE (SEQ ID NO: 1475); LSM12, In, A_7, 20, essential, KKHRSHGRSCYYTPISSGKL (SEQ ID NO: 1476); PSME3, In, A_7, 20, essential, KKNKVFAVWGPLSFLLQLQG (SEQ ID NO: 1477); TAF1, In, A_7, 20, essential, KKSLSFPFRKQHPEEAKALR (SEQ ID NO: 1478); MLLT3, Del, A_7, 20, driver, KKGKRVAQRLYLKVFLAHHH (SEQ ID NO: 1480); MYOSA, In, A_7, 20, driver, KKNWRTRSGPDGEHIPRTDH (SEQ ID NO: 1481); NF1, Del, A_7, 20, driver, KRKTKNQWLASVSLRTASVQ (SEQ ID NO: 1482); PTCH1, Del, A_7, 20, driver, KTAASSWLWASSYLGPSRWD (SEQ ID NO: 1483); RBBP6, Del, A_7, 21, essential, high expression, KKLTMTPESIQVPNVEMKRMN (SEQ ID NO: 1484); SENP6, In, A_7, 21, essential, high expression, KKMFNPFRGFAKKLQTSYYFE (SEQ ID NO: 1485); EIF3J, In, A_7, 21, essential, high expression, KKNSREDKRERTATEEKARRN (SEQ ID NO: 1486); RIF1, Del, A_7, 21, essential, high expression, KMKLANMQNIPLQVYLCQNQI (SEQ ID NO: 1487); FGL1, Del, A_7, 21, high expression, KIAVMHNIRISKLEMKRISTS (SEQ ID NO: 1488); ZNF644, In, A_7, 21, high expression, KKDFHERLCSRIIQKISYLHM (SEQ ID NO: 1490); PLOD2, In, A_7, 21, high expression, KKIPKGKPQSGLCSRWNFVAR (SEQ ID NO: 1492); DMXL2, Del, A_7, 21, high expression, KKISLTSFLTGWMMYLHIQKL (SEQ ID NO: 1493); FEM1C, In, A_7, 21, high expression, KKKRSAWGFEILEKGNEHEVQ (SEQ ID NO: 1494); TOP2B, In, A_7, 21, high expression, KKKSGFWKSLLISFIFSEVRR (SEQ ID NO: 1495); MYO9A, Del, A_7, 21, high expression, KKLSFCNQHVEDSEQDKDLKL (SEQ ID NO: 1496); PHF14, In, A_7, 21, high expression, KKMEPSTKPTTSGFCVHGRAE (SEQ ID NO: 1497); UGDH, In, A_7, 21, high expression, KKNAKASLYLRWTACPGWAPQ (SEQ ID NO: 1498); SMCHD1, In, A_7, 21, high expression, KKNGKYSEVGELYLEITSCVE (SEQ ID NO: 1499); PHF3, In, A_7, 21, high expression, KKNITGSNFSTNFQALNSFFE (SEQ ID NO: 1500); KIF21A, In, A_7, 21, high expression, KKNKGSPNETNERRTRESQTD (SEQ ID NO: 1501); ARHGAP12, In, A_7, 21, high expression, KKNQEKLKEVSYTTPHFASCS (SEQ ID NO: 1502); ZC3H11A, In, A_7, 21, high expression, KKNSSFATHCCQQRTIRGACR (SEQ ID NO: 1503); LUC7L2, In, A_7, 21, high expression, KKNTGSIGENGRYNSSTNQIQ (SEQ ID NO: 1504); OSMR, Del, A_7, 21, high expression, KKQDTLRNLLLQTTLTCWWIH (SEQ ID NO: 1505); KIF21A, In, A_7, 21, high expression, KKRFREVEKKRKEEEKKATET (SEQ ID NO: 1506); GOLGB1, Del, A_7, 21, high expression, KKSRWKKIMKFLLALNKIMMR (SEQ ID NO: 1507); EHHADH, In, A_7, 21, high expression, KKTGFSGKPSPERMAKLGRLP (SEQ ID NO: 1508); STAU1, Del, A_7, 21, high expression, KNQCISLLTLTLGCSPPITTT (SEQ ID NO: 1510); KLF3, Del, A_7, 21, high expression, KNTHPLLQACSPSACRCPCHQ (SEQ ID NO: 1511); TTC17, Del, A_7, 21, high expression, KNVTTSVWDGPARTNASNSAG (SEQ ID NO: 1512); RSF1, Del, A_7, 21, high expression, KRLSKKKVIPSRKMSNPLKLR (SEQ ID NO: 1513); NKTR, Del, A_7, 21, high expression, KTEVKKNPSLNGNALIQKKEL (SEQ ID NO: 1514); MIA3, Del, A_7, 21, high expression, KTLGIRAGSLMLICKSLTEQF (SEQ ID NO: 1515); STIL, Del, A_7, 21, driver, essential, KKHIIQDWWTKIVSLMQLLSN (SEQ ID NO: 1516); NAPG, Del, A_7, 21, essential, KIFIRKLRIIQLVIRKQLLKS (SEQ ID NO: 1517); ANLN, Del, A_7, 21, essential, KIMPFLVKLISNRKCRNSITK (SEQ ID NO: 1518); WDR7, Del, A_7, 21, essential, KKFLHLTRKDGSKLPLLFYLE (SEQ ID NO: 1519); SFSWAP, In, A_7, 21, essential, KKKISNKITFSQVPFVIQVQV (SEQ ID NO: 1520); TCOF1, In, A_7, 21, essential, KKRQRKKRKEEESKKGLNQRF (SEQ ID NO: 1521); SMC6, Del, A_7, 21, essential, KKVLTNLWNLNGWKDKKKYLG (SEQ ID NO: 1522); KMT2C, Del, A_7, 21, driver, KMMRIKIMLTLLAHRLLLTQT (SEQ ID NO: 1523); SF3B1, In, A_7, 22, driver, essential, high expression, KKNCAEGGKTVLWDRWCRSKLH (SEQ ID NO: 1524); BPTF, Del, A_7, 22, essential, high expression, KINHIFDMNLLDMIEVGGNTGS (SEQ ID NO: 1525); CEP350, In, A_7, 22, essential, high expression, KKKAKGKTESPRSQSDQAVRVI (SEQ ID NO: 1526); PRPF40A, In, A_7, 22, essential, high expression, KKRKGTSKAVAKEKLGSLKKHT (SEQ ID NO: 1528); CTR9, Del, A_7, 22, essential, high expression, KNDVHQKQRRKRLPSQNVCLHQ (SEQ ID NO: 1529); TCF12, In, A_7, 22, driver, high expression, KKSQKGASWFAFFCICTIPKFR (SEQ ID NO: 1531); PPP3CA, Del, A_7, 22, high expression, KICWILMRQSLFVGTFMDNSLI (SEQ ID NO: 1532); LARP1B, Del, A_7, 22, high expression, KKCMRNLDNLLGKMQKKITGMG (SEQ ID NO: 1533); CLCN3, Del, A_7, 22, high expression, KKDILRHMAQTANQDPASIMFN (SEQ ID NO: 1534); ROCK1, Del, A_7, 22, high expression, KKFCSIMTNKIRSNPIHLWYWT (SEQ ID NO: 1535); ANK3, Del, A_7, 22, high expression, KKGNTANGPGIGRKSLMPMQVT (SEQ ID NO: 1536); AKAP12, In, A_7, 22, high expression, KKKRRCHSLGIIQKDGDAQEAC (SEQ ID NO: 1537); PLK2, In, A_7, 22, high expression, KKNSSLLRRAWPMLSFSPNRCS (SEQ ID NO: 1538); PIK3C2A, In, A_7, 22, high expression, KKNTCITSYSYSEPFLFSTALF (SEQ ID NO: 1539); ZKSCAN1, Del, A_7, 22, high expression, KKPSQEREVQGRRGKDWEEASV (SEQ ID NO: 1540); ATP2C1, Del, A_7, 22, high expression, KKQVNYQSVKLQAFSKLIFRMV (SEQ ID NO: 1541); CHGB, In, A_7, 22, high expression, KKRGVSGQIGNTCCRAFSGEDT (SEQ ID NO: 1542); DST, In, A_7, 22, high expression, KKSSHCTAFFWCRGFRKIFGRH (SEQ ID NO: 1543); ZMYM4, Del, A_7, 22, high expression, KKTFLNNLIRFLSLNQYGKILV (SEQ ID NO: 1544); CHD2, In, A_7, 22, high expression, KKTRELQEKRGRNQTMVRESFS (SEQ ID NO: 1545); ANK3, Del, A_7, 22, high expression, KKYRRASYPSTKFLLEKNSRRP (SEQ ID NO: 1546); BACH1, Del, A_7, 22, high expression, KNAFHHTVRKQTLNFHFWTRGI (SEQ ID NO: 1547); ZNF700, Del, A_7, 22, high expression, KNGVTRTLNMSTKTPEEASGVS (SEQ ID NO: 1548); WDR60, Del, A_7, 22, high expression, KRKYKKFKELLMQKMKGLASYL (SEQ ID NO: 1549); BRD4, Del, A_7, 22, driver, essential, KKAKPRNLLLKRRRKIIAATAM (SEQ ID NO: 1550); NELFCD, Del, A_7, 22, essential, KISLSLPRWCATGSTRTCLPRP (SEQ ID NO: 1551); NUP88, In, A_7, 22, essential, KKETTRRSQLLSRREEKSAGNG (SEQ ID NO: 1552); POLE2, In, A_7, 22, essential, KKISSSVNDQPPCTKFIWNTKR (SEQ ID NO: 1553); CENPC, Del, A_7, 22, essential, KKLFHLKGRRQQLKATKEYRSF (SEQ ID NO: 1554); ATAD5, In, A_7, 22, essential, KKNHGKFWYPNGFKKWQFTVTH (SEQ ID NO: 1555); NR2C2AP, Del, A_7, 22, essential, KNIFSTRMRRHVGTQTRAPPSG (SEQ ID NO: 1556); ARID2, Del, A_7, 22, driver, KKSLRWVGRSWIFTVSTPESLL (SEQ ID NO: 1557); CHD1, Del, A_7, 23, essential, high expression, KKKLFLVREVQRGEKQELRRIKQ (SEQ ID NO: 1558); CWF19L2, Del, A_7, 23, essential, high expression, KKNIYGIQSLHLLAVQSVSPFTS (SEQ ID NO: 1559); CCT3, Del, A_7, 23, essential, high expression, KNMQEWKRYLEASLKTPVSCVES (SEQ ID NO: 1560); PCM1, In, A_7, 23, driver, high expression, KKNPCQRSSAGAYGRDRKFEETT (SEQ ID NO: 1561); FUBP1, Del, A_7, 23, driver, high expression, KKYKMMLVFAFSLSQMMGQHPKG (SEQ ID NO: 1562); ADAM10, Del, A_7, 23, high expression, KILVSFIFRLIICSLNITEHEKL (SEQ ID NO: 1563); AIDA, Del, A_7, 23, high expression, KKGLPAPSVLLSWRWMKLNLGQL (SEQ ID NO: 1565); ASH1L, In, A_7, 23, high expression, KKHKGGRGPSQTESRKKHRSWER (SEQ ID NO: 1566); NFATC3, In, A_7, 23, high expression, KKKPVSTFYLYTSFDEARTQRRD (SEQ ID NO: 1567); LAMB1, Del, A_7, 23, high expression, KKLKSQLMPEGKPKCYKMKQKLF (SEQ ID NO: 1568); KIAA1462, In, A_7, 23, high expression, KKNERDYILLGFYPSEIRITSAR (SEQ ID NO: 1569); MAP2, In, A_7, 23, high expression, KKNRSSGPLSLQEIHFKTCYQIY (SEQ ID NO: 1570); BAGS, In, A_7, 23, high expression, KKPLHPGSQEKSSDRGANSDHIY (SEQ ID NO: 1571); EEA1, In, A_7, 23, high expression, KKPYTTGASNSTNREAEESVRKS (SEQ ID NO: 1572); CLCN3, In, A_7, 23, high expression, KKRYPPAYGPDGKPRPRFNNVQL (SEQ ID NO: 1573); IQGAP1, Del, A_7, 23, high expression, KKSMIENRPDTRRLASTLDTLIM (SEQ ID NO: 1574); CACYBP, Del, A_7, 23, high expression, KKSRLIQFLYCVERKWKTQGGIT (SEQ ID NO: 1575); CBX3, Del, A_7, 23, high expression, KNRMERVKKLKRQSLKNLSWKKY (SEQ ID NO: 1576); PLOD2, Del, A_7, 23, high expression, KNSKRQTTKWSLQQMEFCGQIKD (SEQ ID NO: 1577); TTF2, In, A_7, 23, essential, KKERFGQTSSIRVWRICHQAHLG (SEQ ID NO: 1578); ZFC3H1, In, A_7, 23, essential, KKSSECRGFLSYVQILHLTESRR (SEQ ID NO: 1579); NUP153, In, A_7, 23, essential, KKTSIQLVCLWNTFPFTWEFFNP (SEQ ID NO: 1580); RUNX1T1, Del, A_7, 23, driver, KKVAAVAAATLGSRVPSTQTQLH (SEQ ID NO: 1581); ESF1, Del, A_7, 24, essential, high expression, KIRKRKKTLFNILQTLLSKKNKGH (SEQ ID NO: 1582); RSRC2, Del, A_7, 24, essential, high expression, KKGKCFGRARKKGTNPNLLKYGKN (SEQ ID NO: 1583); PHAX, Del, A_7, 24, essential, high expression, KKWDQRKRKMGKVISKGNDLSKTG (SEQ ID NO: 1584); CDC5L, Del, A_7, 24, essential, high expression, KSLPLVFMILLRKTTKLLTQISGN (SEQ ID NO: 1585); EIF2A, Del, A_7, 24, high expression, KISWRKFRKKQPFSRSWKIWNWVF (SEQ ID NO: 1586); EPSTI1, Del, A_7, 24, high expression, KKDFKKTLEEKHLESISNTKPLSS (SEQ ID NO: 1587); MAN1A2, In, A_7, 24, high expression, KKDGAFGLLCWGNVCTRSRWFQSR (SEQ ID NO: 1588); ZNF484, Del, A_7, 24, high expression, KKFILEGNTLNVLNVEKLSQGNQH (SEQ ID NO: 1589); ATP13A3, In, A_7, 24, high expression, KKNRYFLYQSSKNKYLWTAQSCLL (SEQ ID NO: 1591); KIF13A, Del, A_7, 24, high expression, KKSAIKQRKQRTMWSGKPSLWSSG (SEQ ID NO: 1592); TRDN, In, A_7, 24, high expression, KKTGKERKTCGTSKVTKERTLSSK (SEQ ID NO: 1593); FAM193A, Del, A_7, 24, high expression, KKWTRSQKGKASLTIGGWRICCSL (SEQ ID NO: 1594); MYH1, Del, A_7, 24, high expression, KMTCNSRFKLKLTAWLMQRKGVTS (SEQ ID NO: 1595); MYH2, Del, A_7, 24, high expression, KMTCSSKFRLKPKAWLMQRKGVTS (SEQ ID NO: 1596); KIAA0232, Del, A_7, 24, high expression, KNLGWKRVLRHHWIPRRNQLGFFQ (SEQ ID NO: 1597); THOC1, In, A_7, 24, essential, KKCCYLEIKYILFCWEKLLTTYVQ (SEQ ID NO: 1599); C11orf57, In, A_7, 24, essential, KKKVTQKTEEKQKGSHRYNSRFLE (SEQ ID NO: 1600); POP1, Del, A_7, 24, essential, KKLSVMELEIHVYHTLGSLQPQAL (SEQ ID NO: 1601); PRIM1, Del, A_7, 24, essential, KNTLKNMPWLIKIFSKIKKAGIRF (SEQ ID NO: 1602); SETBP1, Del, A_7, 24, driver, KKDSPLKRWWKRSCQRKPWLLESP (SEQ ID NO: 1603); ATRX, Del, A_7, 25, driver, essential, high expression, KKQRNLQVQDAFLPTVWALVRLYRW (SEQ ID NO: 1605); SOS1, Del, A_7, 25, essential, high expression, KKLQETMDQVIILHFRVHLPQLSGI (SEQ ID NO: 1606); RPL32, Del, A_7, 25, essential, high expression, KKQSTCCPVASGSSWSTTSRSWKCC (SEQ ID NO: 1607); TBCE, In, A_7, 25, essential, high expression, KKQTQRRIPHSPSQIPVPLPEIWCT (SEQ ID NO: 1608); PIK3R1, In, A_7, 25, driver, high expression, KKNLASHTKAPATSASSCCTRFFEN (SEQ ID NO: 1609); VPS13C, Del, A_7, 25, high expression, KIQLCQKRLYPPEIVTLLISGYLPS (SEQ ID NO: 1610); RSF1, Del, A_7, 25, high expression, KKEGKEKMRWKKSQQLCKKLTKRKF (SEQ ID NO: 1611); ECHDC1, Del, A_7, 25, high expression, KKHFSSFLVDPLTFRRKTMALAFLL (SEQ ID NO: 1612); ZBTB1, In, A_7, 25, high expression, KKKFRVQIIYSKRTCVKTLWAEFYL (SEQ ID NO: 1614); UGP2, In, A_7, 25, high expression, KKNTTEVQSLSCENLHFQSKQVPED (SEQ ID NO: 1615); RANBP9, In, A_7, 25, high expression, KKNVEGCIQSTSIFRSLEQPSWKSA (SEQ ID NO: 1616); EIF2A, In, A_7, 25, high expression, KKSVGENSERNSPSPGAGRFGIGYL (SEQ ID NO: 1617); TTN, Del, A_7, 25, high expression, KNRPLKLFLEKSHQLKYRRCLRKLW (SEQ ID NO: 1618); USP33, Del, A_7, 25, high expression, KNSTRNTEVLFQTYLMEQSLVQCSV (SEQ ID NO: 1619); RGL1, Del, A_7, 25, high expression, KSSLSPPHPVLLSIPWTQIPQGCLP (SEQ ID NO: 1620); ZBTB1, Del, A_7, 25, high expression, KSSYLNILPALFDVLIVASVLKLKI (SEQ ID NO: 1621); ASH1L, Del, A_7, 25, high expression, KTQRRKRTFANGIEKETSKLGKMMV (SEQ ID NO: 1622); C12orf65, Del, A_7, 25, essential, KKEQRKPWKKRSYLKNCGSQVKRST (SEQ ID NO: 1623); DNMT1, In, A_7, 25, essential, KKNAPGEEEETEQESHLLGRRSRQD (SEQ ID NO: 1624); WDR7, In, A_7, 25, essential, KKNFYILRGKTEASYRYCFTWSNRS (SEQ ID NO: 1625); XPOS, Del, A_7, 25, essential, KKQSQCWRRRCWTMMGVAWPPSLNP (SEQ ID NO: 1626); NCAPG, In, A_7, 25, essential, KKTAGCFTGDSYFTHNPNIPGFFSC (SEQ ID NO: 1627); GEMIN5, Del, A_7, 25, essential, KKVLNWRKNGSLNLRQSPKRRKSPP (SEQ ID NO: 1628); ALG8, Del, A_7, 25, essential, KKWVKNLQKSQNLFCRYYFCGTSGY (SEQ ID NO: 1629); PWWP2A, Del, A_7, 25, driver, KRKLEKVVVQLTLLNMKIKNGEMKV (SEQ ID NO: 1630); CTNNBL1, Del, A_7, 26, essential, high expression, KKGCCRLLTEMGKRKRKRRSHWMKAQ (SEQ ID NO: 1631); RBBP6, In, A_7, 26, essential, high expression, KKNQTQQRNWEENWKYRKYIKHKRTL (SEQ ID NO: 1632); XRN1, In, A_7, 26, essential, high expression, KKNTTGMCSRRNYISPSTLVFNERVY (SEQ ID NO: 1633); AKAP9, Del, A_7, 26, driver, high expression, KKCMSSSPKWKIFSASWKRKDNKFIS (SEQ ID NO: 1634); ZCCHC6, Del, A_7, 26, high expression, KIHLWISLIFFMNSVNLSSPKASLLR (SEQ ID NO: 1635); SYNE2, Del, A_7, 26, high expression, KIKSCVPGWCRWKTKFYRQRTLVLKK (SEQ ID NO: 1636); CEP170, Del, A_7, 26, high expression, KIQDILQAKETEWHKVRAREEKLRKF (SEQ ID NO: 1637); SECISBP2L, Del, A_7, 26, high expression, KIRHFLEVEGKLNKEIIHRMKMGFKN (SEQ ID NO: 1638); RGS2, Del, A_7, 26, high expression, KKANSKLSSSLLLRKHSCGQKHLTSC (SEQ ID NO: 1639); ALCAM, In, A_7, 26, high expression, KKKHDCFNSYHSSLFGFVLKPKWRSD (SEQ ID NO: 1640); RAB2A, Del, A_7, 26, high expression, KKKKVKLLHENMDSSSWKRLLRLLPM (SEQ ID NO: 1641); PARK7, Del, A_7, 26, high expression, KKRDHMMWWFYQEVIWAHRIYLSLLL (SEQ ID NO: 1642); MGA, In, A_7, 26, high expression, KKRGPETTIFLLLPISIISAANFMSF (SEQ ID NO: 1643); RBM34, Del, A_7, 26, high expression, KKRNGMRRKKVHPRLKDHFRKNLPKK (SEQ ID NO: 1644); PALLD, In, A_7, 26, high expression, KKSTALSQSLCSTFGPGTRHQSSVPT (SEQ ID NO: 1645); GIGYF2, In, A_7, 26, high expression, KKSTERAYSRRAGDGLPACGRRGGVL (SEQ ID NO: 1646); AKAP12, Del, A_7, 26, high expression, KMKTLPLIQGKMLCPQGPTVRQNRHQ (SEQ ID NO: 1647); ITSN1, Del, A_7, 26, high expression, KNLKLPRLLPHTPPPAPSSSLSPLVS (SEQ ID NO: 1648); TRIP12, Del, A_7, 26, high expression, KNQKHFSILNLPQKQISHIVSQRRDI (SEQ ID NO: 1649); NCAPH, Del, A_7, 26, essential, KKILKLTLKMILTLMYILEKQRLLLF (SEQ ID NO: 1650); ORCS, In, A_7, 26, essential, KKTRKDKQSSPWAKTISTRQIISNII (SEQ ID NO: 1651); THOC1, Del, A_7, 26, essential, KMLLLGNQIHSILLGKITYYVCAMIS (SEQ ID NO: 1652); GRIN2A, In, A_7, 26, driver, KKKLPFPPRSGGQQEEQVSLARPHLR (SEQ ID NO: 1653); EP300, Del, A_7, 27, driver, essential, high expression, KKCLTRLYQSVLSMTTRIFLNKLLKID (SEQ ID NO: 1654); CTCF, In, A_7, 27, driver, essential, high expression, KKNKENQKEQTALYRGGQRCRCVCLRF (SEQ ID NO: 1655); MPHOSPH10, In, A_7, 27, essential, high expression, KKAVAASGGSDSTEEAREQPPGGDPTL (SEQ ID NO: 1656); ZNF131, Del, A_7, 27, essential, high expression, KKEGRSSTNARSATVCLTAGTSSKITW (SEQ ID NO: 1657); SRRM1, In, A_7, 27, essential, high expression, KKGGHEQSKFGGYKALDNKKSNGNPWV (SEQ ID NO: 1658); ZNF131, Del, A_7, 27, essential, high expression, KKGRLQKLQMLSLSHCHLQNQNLLKLR (SEQ ID NO: 1659); STRAP, Del, A_7, 27, essential, high expression, KKLCGAVRINRFFLLMTKLFDFGIMLL (SEQ ID NO: 1660); SMARCAS, Del, A_7, 27, essential, high expression, KKMRSRTYYPLAITDTVEQSKRRMKSY (SEQ ID NO: 1661); NMD3, In, A_7, 27, essential, high expression, KKNFLLSGTVNSEIWNASEYTSYQRDS (SEQ ID NO: 1662); CCTS, Del, A_7, 27, essential, high expression, KKWKMRRLQFSHVHLNHPNQKQSISWM (SEQ ID NO: 1663); ANK2, Del, A_7, 27, high expression, KIHRMSRNGSRKGWLILLITLASAGQN (SEQ ID NO: 1665); ZCCHC6, In, A_7, 27, high expression, KKEDNTRELWEYPQKRTMCCFQQSICI (SEQ ID NO: 1666); SCAPER, Del, A_7, 27, high expression, KKFSSSMMKVFEGTWNRLNKEKKKLLS (SEQ ID NO: 1667); SAMHD1, In, A_7, 27, high expression, KKGMERQYFSPKSNSPPRSIQKQSPAF (SEQ ID NO: 1668); AKAP12, In, A_7, 27, high expression, KKIKVQAGREKRRLHSWVWCRTFHSRH (SEQ ID NO: 1669); CLCN3, In, A_7, 27, high expression, KKKGGAISCLSCRGFCSFWCTNWRSSF (SEQ ID NO: 1670); ANKRD12, Del, A_7, 27, high expression, KKLTTERKVEKRWIGNMTKKSLKKRGI (SEQ ID NO: 1671); TRDN, Del, A_7, 27, high expression, KNRKRKKNMWNQQSHQRKNTQFQVTNK (SEQ ID NO: 1672); BRCA2, In, A_7, 27, driver, essential, KKNRTCPFRLFVRRMLQFTGNKVLDRP (SEQ ID NO: 1674); NOL8, In, A_7, 27, essential, KKESPECCTKCFANQLKQFYKQRISSC (SEQ ID NO: 1675); ATAD5, Del, A_7, 27, essential, KKSWKILVSKWFQKMAIYSYTLIKEVF (SEQ ID NO: 1676); CHD4, Del, A_7, 28, driver, essential, high expression, KNCMTCWGRTCCGGSKPMCSRTCPPRQN (SEQ ID NO: 1678); HECTD1, Del, A_7, 28, essential, high expression, KKLQQKYYSRLRNHWHWQVGLCQTGVNN (SEQ ID NO: 1679); ALMS1, Del, A_7, 28, high expression, KKARCFLIIELGGLIKLKLNRLNLINIF (SEQ ID NO: 1681); USP15, Del, A_7, 28, high expression, KKDILMKMLLRTLKNMKVWSINLLKNPL (SEQ ID NO: 1682); CETN3, Del, A_7, 28, high expression, KKEENCLRNRNKKLKMLLNYLIQTKMKQ (SEQ ID NO: 1683); ZHX3, Del, A_7, 28, high expression, KKHHSRRMSLASLWVRPYQSCRLEKWR (SEQ ID NO: 1684); VCPIP1, In, A_7, 28, high expression, KKIAGNGFFYSGFNGQAPSGSKYRAVTI (SEQ ID NO: 1685); NKTR, In, A_7, 28, high expression, KKKEETNSFRRLGFLFQFLLFFRIIFRK (SEQ ID NO: 1686); TRDN, Del, A_7, 28, high expression, KKKYTKIRLKNKRNLKGKYKLKKLDTAL (SEQ ID NO: 1687); PLK2, Del, A_7, 28, high expression, KKQFTITQSLANAQFSQQQMLLSNLLVK (SEQ ID NO: 1688); PAPOLA, Del, A_7, 28, high expression, KKQKTLKTSVLISPMIFSLSQIQFIGKQ (SEQ ID NO: 1689); ADAMTS1, Del, A_7, 28, high expression, KKYQDQLLVQNLDIMISSQFQLEPPTSK (SEQ ID NO: 1690); MYO9A, In, A_7, 28, high expression, KKYRSYAPRHCSSSEKQQECIYLWDDWN (SEQ ID NO: 1691); MASTL, Del, A_7, 28, essential, KIKTLQTSFQPVCLKHHSFLKDSYALCL (SEQ ID NO: 1692); NHLRC2, Del, A_7, 28, essential, KKGIYFMLQTPTITRLKLWIQKQKTVQH (SEQ ID NO: 1693); TICRR, Del, A_7, 28, essential, KNWIRKTKLESASFRYFFVWRCVCNALQ (SEQ ID NO: 1694); IL7R, Del, A_7, 29, driver, high expression, KKGLSLSYGPVSPIIRRLWNIFVRNQEKI (SEQ ID NO: 1695); ZNF43, Del, A_7, 29, high expression, KKFILEENPTNVKNVAKLLTSSQLLLNIR (SEQ ID NO: 1696); ZNF708, Del, A_7, 29, high expression, KKFILERNPISVKNVAKALFCPLILLHIR (SEQ ID NO: 1697); ZNF117, Del, A_7, 29, high expression, KKFILERNPTNVRNVAKLLTNSQTLLDIR (SEQ ID NO: 1698); DDX50, In, A_7, 29, high expression, KKKPLTKGTCFGSNKGTGKPSSQRLQRYN (SEQ ID NO: 1699); ZNF486, Del, A_7, 29, high expression, KKLILERNHTNVKNVAKPSTGPHTLLHIR (SEQ ID NO: 1700); KIDINS220, In, A_7, 29, high expression, KKNMLSPIFCHLPFYHWLHYIWNYSSGYI (SEQ ID NO: 1702); OAT, Del, A_7, 29, high expression, KKQSKALQPLMTFLKGNISMVHTTTILYL (SEQ ID NO: 1703); ZC3H14, In, A_7, 29, high expression, KKSRNSTEAIIIPTANRPSNGRNSADESS (SEQ ID NO: 1704); ZCCHC11, Del, A_7, 29, high expression, KKSSANVIALICLSRLTQINLLEQTAGQI (SEQ ID NO: 1705); GPBP1, In, A_7, 29, high expression, KKWMAYTWKKWYRKHKSSRWIPWWKFPFS (SEQ ID NO: 1706); GLS, Del, A_7, 29, high expression, KNLILEEKVVIKGIPLDHWTMKVSNKNLL (SEQ ID NO: 1707); MAP3K2, Del, A_7, 29, high expression, KNRMMSESNLNIEEKKESFSSPDQLNWKI (SEQ ID NO: 1708); DDX52, In, A_7, 29, essential, KKKEDDDFRNCFPRRRCYYTVDVICRSKD (SEQ ID NO: 1709); MBTPS2, In, A_7, 29, essential, KKKLKFKFLYNTFFGNSHSLNKSKTPTSD (SEQ ID NO: 1710); DNMT1, In, A_7, 29, essential, KKNGSRQNSHELQDPPSQVHSVRAVPGRP (SEQ ID NO: 1711); MMS22L, In, A_7, 29, essential, KKSFQCRAAADLWNDGNSCEIMGTNLCHF (SEQ ID NO: 1712); NF1, In, A_7, 29, driver, KKFISLSVDYIGYTGKMSCWATKGHNEIR (SEQ ID NO: 1714); CACNA1D, In, A_7, 29, driver, KKNAASTAAASGPRERGKLCKRHQTSSFW (SEQ ID NO: 1715); ATRX, Del, A_7, 30, driver, essential, high expression, KKEFQIQKILTLLKMRNTAKKEWIIKGTKI (SEQ ID NO: 1716); METAP2, Del, A_7, 30, essential, high expression, KKDERRRRAKGLLQQGNRNLIKNQEPQWMK (SEQ ID NO: 1717); KRR1, Del, A_7, 30, essential, high expression, KKLLRKNIRHSHHHNQKVRSIKNWLVVNTF (SEQ ID NO: 1718); EXOSC10, Del, A_7, 30, essential, high expression, KKLNSRWETKACPFQLESQTEASGTTGHRD (SEQ ID NO: 1719); BDP1, In, A_7, 30, essential, high expression, KKLSAGRESTKGFKTCTSEGPIAKAKAKCR (SEQ ID NO: 1720); DDX42, Del, A_7, 30, essential, high expression, KKSLILFPPLIIQRLTIHHLKKTFTMSMKR (SEQ ID NO: 1721); PAFAH1B1, Del, A_7, 30, essential, high expression, KKVVNLGHSCCLDPETRLLRCGMSVLACAL (SEQ ID NO: 1722); ERC1, In, A_7, 30, driver, high expression, KKEECTNVRGGATTGGQSQRQLSAATGQSP (SEQ ID NO: 1723); ZKSCAN1, In, A_7, 30, high expression, KKNHHRRERSKGEGERIGKKLQSELQLHHP (SEQ ID NO: 1725); RNF13, Del, A_7, 30, high expression, KKPVQCASKKLFLLKAIQTLTQTVVKKKMK (SEQ ID NO: 1726); NASP, Del, A_7, 30, high expression, KKQKTSLWQSLKLIKNRTVKWRRVEEKIWI (SEQ ID NO: 1727); KIF21A, Del, A_7, 30, high expression, KKVWLVKRIIQTLTKRRKKKRVFRKEKTMN (SEQ ID NO: 1728); UTRN, Del, A_7, 30, high expression, KNLSRNCLLLLPQRRDRSMWILKLRKSLML (SEQ ID NO: 1729); SYNE2, Del, A_7, 30, high expression, KNRISPLLTILEIFYLKKRKQLLSFQQICQ (SEQ ID NO: 1730); CHD3, Del, A_7, 30, high expression, KRRRRQSGGKRGREMGGKSKWNRSHQQLCF (SEQ ID NO: 1731); NAA35, In, A_7, 30, essential, KKNKEKKESSPIEPRDHNEPSISEHVCWNV (SEQ ID NO: 1732); ARID2, In, A_7, 30, driver, KKNPCGGWEGAGSSRSLHQSHYFRRIREGF (SEQ ID NO: 1734); CTCF, Del, A_7, 31, driver, essential, high expression, KKSQMLSPQRERKRRPSQLPQMPPTETSRPR (SEQ ID NO: 1735); RBM39, In, A_7, 31, essential, high expression, KKQSCSNGKQFTKGKCWTYEALCGLITLQHN (SEQ ID NO: 1736); VPS35, In, A_7, 31, essential, high expression, KKSSKNSKSVHGPLSTSAAFYRNSEQIYLFL (SEQ ID NO: 1737); RBBP6, Del, A_7, 31, essential, high expression, KKVILVPQETENLMITKPLMILNGQMKRQNL (SEQ ID NO: 1738); DENR, In, A_7, 31, high expression, KKEDRTTKGYYSQNSQSKEEICDKSMWPCNF (SEQ ID NO: 1739); ZC3H14, In, A_7, 31, high expression, KKSRNSTEAIIIPTANRPSNGRNSADESRLL (SEQ ID NO: 1740); TRIM22, Del, A_7, 31, high expression, KNSRSSVRRMEKSFAGFVNCLRNTKVTKHSA (SEQ ID NO: 1741); NKTR, Del, A_7, 31, high expression, KRLSIKRKGKSRNTAEDTNKQRREGFLYRLT (SEQ ID NO: 1742); MEAF6, Del, A_7, 31, high expression, KTPIAKMIEGTGSLRKLSGSSVNPRLPQQLQ (SEQ ID NO: 1743); EIF2B5, In, A_7, 31, essential, KKCFCDDDDLQGVIPQPPNSLPRRQCGSGCG (SEQ ID NO: 1744); HAUS1, In, A_7, 31, essential, KKFNCNFSIRKMSTRGCQESRVASVYRKGQS (SEQ ID NO: 1745); TTF2, Del, A_7, 31, essential, KRKIWPNKFYQGLENLSPSSPWLTSESFLAS (SEQ ID NO: 1746); RANBP3, Del, A_7, 31, essential, KTCRIRERSLGGRLRPPTMARVTPSQLASMP (SEQ ID NO: 1747); MECOM, Del, A_7, 31, driver, KKEATSNQDLLQMVPCSMQDPLLSLWTLFTE (SEQ ID NO: 1748); ZRANB2, Del, A_7, 32, essential, high expression, KKDEQDHGHPKDATGHHLDHPILVPVQVQKRN (SEQ ID NO: 1749); CFDP1, In, A_7, 32, essential, high expression, KKKKGPEHSSQEEKTRWPLIRRRGRGGCQFRI (SEQ ID NO: 1750); CWF19L2, Del, A_7, 32, essential, high expression, KTMSHLHHQALKMSGLRLFHPRLLTRKKPGK (SEQ ID NO: 1751); MSH6, Del, A_7, 32, driver, high expression, KNEGSYQILRVTLVALMWNLSQTLRRKEAVMK (SEQ ID NO: 1752); BRE, Del, A_7, 32, high expression, KIIRTAPDGMEMKWPKEQKERATEMGRNPALP (SEQ ID NO: 1753); ARID5B, In, A_7, 32, high expression, KKNRRVSGIFSEAPGIQSRPREGQRNRPRFQQ (SEQ ID NO: 1754); HDAC2, Del, A_7, 32, high expression, KKQTLRKKINPRTTVVKKQIPKEPNQNSSATP (SEQ ID NO: 1755); WASL, In, A_7, 32, high expression, KKSGAEQSASVLLWTRCTVRPDTTGYPTKICG (SEQ ID NO: 1756); TRIP12, In, A_7, 32, high expression, KKTYCEPRGAAETGGVCDAGPRQLTGHVRNPV (SEQ ID NO: 1758); PAM, Del, A_7, 32, high expression, KRILVDLMPERVQNMRGVMLFLSETEFTNSTD (SEQ ID NO: 1759); SAMHD1, Del, A_7, 32, high expression, KRNGTTVLQSKIQLASEKHPKAESSFLKMTQC (SEQ ID NO: 1760); UTRN, Del, A_7, 32, high expression, KRPLMKSLRIRNLHYINLQKKQRLWRKMFILM (SEQ ID NO: 1761); OGFRL1, Del, A_7, 32, high expression, KRRVYLLRITKKVEMITKTMKILEIQIAMMLY (SEQ ID NO: 1762); KMT5A, In, A_7, 32, essential, KKSSRKNATESQTYGFLPCPKELQEEQSRAAV (SEQ ID NO: 1763); NCAPG, Del, A_7, 32, essential, KNCWLFYRRFLFYPQSQYPWFLFLLKDYSTSL (SEQ ID NO: 1764); ATAD5, Del, A_7, 32, essential, KNKLSTKYFLPSVIVNKNWRLMSAIKKPKGNS (SEQ ID NO: 1765); ZRANB2, In, A_7, 33, essential, high expression, KKKTNKITVTRKTPQVIIWIIPFWFPFKFKKEI (SEQ ID NO: 1766); PHAX, In, A_7, 33, essential, high expression, KKNGIKGRGKWARSSQKETTCQRQAREQTRNEL (SEQ ID NO: 1767); DLD, In, A_7, 33, essential, high expression, KKSSRKDGCYWCRSNRCRIGFSLAKTWCRCDSS (SEQ ID NO: 1768); DNTTIP2, Del, A_7, 33, essential, high expression, KTTVFHHIVNQSINFRKNAEKNDKKQQGMAGLV (SEQ ID NO: 1769); PIK3R1, Del, A_7, 33, driver, high expression, KKSRLPHQSPGHLGLFLLHQVLRKLKQMLNNKL (SEQ ID NO: 1770); ZCCHC6, Del, A_7, 33, high expression, KIHLWISLIFFMNSVNLSSPKARDWTVSELLKN (SEQ ID NO: 1771); PPFIA1, Del, A_7, 33, high expression, KIVNFKKSMNCWRKPGDKVYLLPNGTGQRLWSG (SEQ ID NO: 1772); VPS13B, Del, A_7, 33, high expression, KKFPPSAQFIMSCIIRFPVIRTARPKTYFQAYF (SEQ ID NO: 1773); ZEB1, Del, A_7, 33, high expression, KKKIQSLQTVVKVKSYQKILLLSLRRTKALKGG (SEQ ID NO: 1775); CDR2, In, A_7, 33, high expression, KKNSDNVAGPAEPGAAEAGDYGGGIWARVKGEQ (SEQ ID NO: 1777); POLE3, Del, A_7, 33, high expression, KKQTRKSKTRAGMRTMMKTKKGWKKKNRMKRKK (SEQ ID NO: 1778); ATP2C1, Del, A_7, 33, high expression, KKQVNYQSVKLQAFSNLKIPLLCCFWLLQSSVF (SEQ ID NO: 1779); TCF20, In, A_7, 33, high expression, KKTEAKEGEKEAWGPAEEAKNQTSSSHCGTPRT (SEQ ID NO: 1780); RFC1, Del, A_7, 33, essential, KMSKEKEKLVHLKRNQNLKRAGRLPKGTVWQRQ (SEQ ID NO: 1781); CHD4, In, A_7, 34, driver, essential, high expression, KKGAYALMPAAGGQLWGGARVCGGGGRGGSALRQ (SEQ ID NO: 1783); KIAA0232, In, A_7, 34, high expression, KKIWDGKECSDITGFPGGINWDSFSRKAKSVFGM (SEQ ID NO: 1785); TRDN, Del, A_7, 34, high expression, KKKYTKIRLKNKRNLKGKYKLKLHTKKKKKEKKK (SEQ ID NO: 1786); CAPN3, In, A_1, 34, high expression, KKNQAHHLRFGQSKQQQGAGCGPGVRGGQRQNKP (SEQ ID NO: 1788); MYOM1, In, A_7, 34, high expression, KKNSFVCYRPENPEHSRGHPTVLFCNLLCGGFES (SEQ ID NO: 1789); LAP3, Del, A_7, 34, high expression, KRRWLCRQSSMEVGIRRPGRKESCLLLGRTWHAN (SEQ ID NO: 1790); USP24, Del, A_7, 34, high expression, KTRRMDSSHLSLIIPSLYGWYQLCVSSMKLLAHS (SEQ ID NO: 1791); RHOU, Del, A_7, 34, high expression, KTSKRSLMQPSSLAFNTRTLSNSQRSLKAGLQIK (SEQ ID NO: 1792); TTK, In, A_7, 34, essential, KKAAAFRGGKEEFISIYGINCPRIIFRFTWAFTE (SEQ ID NO: 1793); REV3L, In, A_7, 34, essential, KKNCDYALQMCPKSTTGSSVASSQRRIRTFQETA (SEQ ID NO: 1794); NDC80, In, A_7, 34, essential, KKNGFGGYFRTIECNDNRKQEKCENSERRSSKAG (SEQ ID NO: 1795); ABL2, Del, A_7, 34, driver, KNQRKVLLQAGRDQKPSYCPEEPQLFLSEHPLGI (SEQ ID NO: 1797); USP8, In, A_7, 35, driver, essential, high expression, KKQTRKRTSGKAARGTERETEEGRTRTKSQKETRS (SEQ ID NO: 1798); BDP1, Del, A_7, 35, essential, high expression, KIESPLICFHLQVLLLLNLRILAAQQLRFLVISPY (SEQ ID NO: 1799); XRN1, Del, A_7, 35, essential, high expression, KKGLQTKRTLRIRKPRVLKPLQFRLASQILPTLSK (SEQ ID NO: 1800); PSMC2, Del, A_7, 35, essential, high expression, KKPALSSLMKLMLLEGLVLMMVLEVTMKCREQCWN (SEQ ID NO: 1801); SORL1, Del, A_7, 35, high expression, KKCPLLLVYIMSTTGWLERAYGRLWRPTAIRQTLY (SEQ ID NO: 1802); MPDZ, In, A_7, 35, high expression, KKEQLPVSDGPTVWLPRTGVHPKYKQIINTSNFCF (SEQ ID NO: 1803); KIDINS220, Del, A_7, 35, high expression, KKHVVSHLLSSSFLSLAALYLELLFWLYLELTQSI (SEQ ID NO: 1804); TRDN, In, A_7, 35, high expression, KKTCRSRTTQGKKTGKERKTCGTSKVTKERTLSSK (SEQ ID NO: 1805); NIPBL, Del, A_7, 36, essential, high expression, KKENKILTHRRLGVLQEVIDQLLRRRVLREMGQGQH (SEQ ID NO: 1811); SRRM1, In, A_7, 36, essential, high expression, KKFQKRKGEGEDPTTISVTLQIKIPDAVPLSFSHST (SEQ ID NO: 1812); AHCTF1, In, A_7, 36, essential, high expression, KKNRGSCTAERISFGFIFSVCHLTSCFKEQTKKHIQ (SEQ ID NO: 1813); CTR9, In, A_7, 36, essential, high expression, KKTTSTKSREEKGSQARTSASINEGKNKIQSHNFIK (SEQ ID NO: 1814); BPTF, Del, A_7, 36, essential, high expression, KNGSVKRVWSRNVASRMPLSCQLCSSSTKSSSEPRS (SEQ ID NO: 1815); GOLGA4, Del, A_7, 36, high expression, KISTIRRLQSWLKNTRQNWKALSISRMPFGLKNSKS (SEQ ID NO: 1816); MTSS1, Del, A_7, 36, high expression, KKGEVISSLSWTVLSKMSMISISYWKKQKSRLSGRL (SEQ ID NO: 1817); NOSTRIN, In, A_7, 36, high expression, KKHGGLFYQTEMGKHTRELLPEHSGAGEGKNSTFMQ (SEQ ID NO: 1818); USP47, Del, A_7, 36, high expression, KKHGRILALSFWIIIFMKKILIFPATGRFSLKFLMG (SEQ ID NO: 1819); PHIP, In, A_7, 36, high expression, KKKDLKTPAKIRKLYLCILYTYTINTAKTQCCSDKR (SEQ ID NO: 1820); OSMR, In, A_7, 36, high expression, KKNRILSGTCSFRQPSRAGGYIDIPLLHSELERLLY (SEQ ID NO: 1821); MAP4, Del, A_7, 36, high expression, KKQRQLLQPESLNLMQSLKQPAQLQVHRNNLRGKSR (SEQ ID NO: 1822); MAP1B, In, A_7, 36, high expression, KKTSCFKTKSTQEGRVCQERFCCCRKAKGEGENKSH (SEQ ID NO: 1823); NDUFS4, Del, A_7, 36, high expression, KMDGAMTLKRGRFQNPSPSLMVQTFLGTKEQEYPQN (SEQ ID NO: 1824); WASL, Del, A_7, 36, high expression, KNFEKQLQTFWAVDKGNLRKDEIPQMVLIYPWLQLI (SEQ ID NO: 1825); TCF20, Del, A_7, 36, high expression, KNRGKGGREGSLGPSRGSEKPNKQFPLWNPKNLRSN (SEQ ID NO: 1826); MBP, Del, A_7, 36, high expression, KRETWVNFHGQPQRTTKCSERQMRTRTMGPPLRTQR (SEQ ID NO: 1827); TRRAP, Del, A_7, 36, driver, essential, KKHKRTRPLLSRPPGSQRTWTASNWCPWFRKPSPPS (SEQ ID NO: 1829); SUGT1, In, A_7, 36, essential, KKLCCCPRNFYRRTKIRYRDGVSSCWPGWSPTLDLK (SEQ ID NO: 1830); USP8, Del, A_7, 37, driver, essential, high expression, KKDLISSNSRIISIQYLDLETSKKLSKKLKDSLKALN (SEQ ID NO: 1831); GMPS, Del, A_7, 37, driver, essential, high expression, KKVSEKMEFSTLVWIIHVHYSGAFRRKKLFCLHMEIV (SEQ ID NO: 1832); CEP350, In, A_7, 37, essential, high expression, KKVKTTTGKNQLTDRQFTKSLCKGHSQSTTTNQKNQG (SEQ ID NO: 1833); TCERG1, Del, A_7, 37, essential, high expression, KNGRETIIKTLTQRKKLPWKLKLKLPEKGPLSLWRLE (SEQ ID NO: 1834); RNF213, In, A_7, 37, driver, high expression, KKPGSRCPGSEGKHAEPGWRSHRVLPRHHLSSFPIQS (SEQ ID NO: 1835); LARP4, Del, A_7, 37, high expression, KIGELSSEVEDDEKMTGSQDLILQQLNQRLQHQSLTY (SEQ ID NO: 1836); SP110, Del, A_7, 37, high expression, KKASQEGQPHLDTESKRSSKGWIRFLKRKMTQLVTPR (SEQ ID NO: 1837); PPP2R5A, Del, A_7, 37, high expression, KKLKSHFSSRYPSVYPVLIFRLQKGHCTSGITNIFLV (SEQ ID NO: 1838); HP1BP3, In, A_7, 37, high expression, KKNNSFLGYPFCQPASQGPETNTDGFFPTSQDGCNLN (SEQ ID NO: 1839); NAIP, In, A_7, 37, high expression, KKPLIPDLPIDCCPYKQGQGHPPIPRDHSRDQSISLL (SEQ ID NO: 1840); ZNF43, Del, A_7, 37, high expression, KKTIKVWMSVRCTEEVIMDLTNVCQLPRAKYFYLINV (SEQ ID NO: 1841); ZBTB1, Del, A_7, 37, high expression, KKVPCPNYLLQKNVCQDALGGVLPVIAVDLALAVKNY (SEQ ID NO: 1842); ATF4, Del, A_7, 37, high expression, KKWSKTRQQPLGTARRRGRSRRLLLVSAKSWKRRTRL (SEQ ID NO: 1843); ZBTB38, Del, A_7, 37, high expression, KMKKGMKNQPSLMGQGSQMHFSPSKQKIVITCFPRWT (SEQ ID NO: 1844); VCPIP1, Del, A_7, 37, high expression, KNCRKWFLLFRLQWTGTFGIKVQSSHHLIFLKGKQKL (SEQ ID NO: 1845); TMEM131, Del, A_7, 37, high expression, KNQRLQTFILILDYSVGIIAMLACLFYPNLNPKCSLV (SEQ ID NO: 1846); CASP4, Del, A_7, 37, high expression, KRNITMLKLKTKFGSWQTLCKRSNVWQDKCFFKPFLT (SEQ ID NO: 1847); GTPBP10, In, A_7, 37, essential, KKHDSREDCRVPTYHPHICSYWRRNRRIKELYKKVTG (SEQ ID NO: 1848); KMTSA, Del, A_7, 37, essential, KKLKEKRNRIANLRISTLSEGAPGRAKPSCSLKKGKE (SEQ ID NO: 1849); SOS1, Del, A_7, 38, essential, high expression, KKVTMAMPSSQTALPPLHHLLLKHLLLTAQEGICHHHH (SEQ ID NO: 1851); PRDM2, Del, A_7, 38, high expression, KKDLRLRIMYTCGRCITQIWDGCALMPLIQRRETGCDM (SEQ ID NO: 1852); FKBP15, In, A_7, 38, high expression, KKGNRAADAADRKPEGDRSSQGPAHQSAGKALRAPRNL (SEQ ID NO: 1853); ZHX1, Del, A_7, 38, high expression, KKHLSSCTCLRVHLSGHSGHHQKSMTSWPKKAGLLEQT (SEQ ID NO: 1854); HIST1H1D, Del, A_7, 38, high expression, KKLPRVQLRPKPLSPRRPSLSRGSRRLQRQRRQLRRKS (SEQ ID NO: 1855); OSBPL8, Del, A_7, 38, high expression, KKNLLRYKRKITEKKRKEPQRSCSVQSQILLLLLWLIG (SEQ ID NO: 1856); G3BP2, Del, A_7, 38, high expression, KKQELQESEKPEVVVMIAGILGAMIEVPVVHVELWVVE (SEQ ID NO: 1857); MYH6, In, A_7, 38, high expression, KKQGSSQRDCCGPVPEVLPQAHGHSLLLLRNCRYWGQW (SEQ ID NO: 1858); TACC3, Del, A_7, 38, essential, KIQKIATSCFRHQKLPEDRLFFVCHRKKMCHPRTWPKL (SEQ ID NO: 1860); RAD17, Del, A_7, 38, essential, KNLIGFLKIKRSKLLVAKMFLCFSSELWGKFYIVKEHL (SEQ ID NO: 1861); WRN, In, A_7, 38, driver, KKKYHGTVTRKSLQFLTACYFGTRAGDSDCVIWQIGRS (SEQ ID NO: 1862); MECOM, In, A_7, 38, driver, KKRKQRRIKTCFRWFLAACKTHSFLYGPYLQSREKKTN (SEQ ID NO: 1863); CHD1, Del, A_7, 39, essential, high expression, KRDRLIHLRRMMMKKIMIMIKEVLVAKQLLMLAIRRMKK (SEQ ID NO: 1864); BRE, Del, A_7, 39, high expression, KIIRTAPDGMEMKWPKEQRLISKPLSLSSRRQHLPMESS (SEQ ID NO: 1865); C1S, Del, A_7, 39, high expression, KRAGNFAIMEIQCPALRKTLPILFGSLRRQNMSLEMWCR (SEQ ID NO: 1866); ANKRD12, Del, A_7, 39, high expression, KTSKMINQPRKSMCQKRGTLKRNETRLKRKARNLLGRKK (SEQ ID NO: 1867); PRPF40A, Del, A_7, 40, essential, high expression, KSKPLMPIKSRQKKKKKKKQDQSTKRLRNPFSVFLKIMRK (SEQ ID NO: 1868); KNG1, In, A_7, 40, high expression, KKNLPYCQLSTTGNDLTDEKASRFFTFPIITNRGNKRRNN (SEQ ID NO: 1869); SLC23A2, Del, A_7, 40, high expression, KNTDASATYPSAQPLWATHGKASGRATTAGVQMKTPRPRD (SEQ ID NO: 1871); NOC3L, In, A_7, 40, essential, KKIYDFQRKEKISIKNAEKVEESRRETRARASRGRSFREY (SEQ ID NO: 1872); TCOF1, Del, A_7, 40, essential, KKTKKKKKRRRKQKRPQPKILSHRPRRKRRKRRRQQSRLY (SEQ ID NO: 1873); TSC1, Del, A_7, 40, driver, KTEAMFSSRLRGLIPPKNGFWNWNLTWPRKTTFFWNRRNI (SEQ ID NO: 1874); SPEN, Del, A_7, 41, driver, essential, high expression, KKSNWTDLILLPAPKTVRSLPVFLLGLAQGPAQTYKQDWEN (SEQ ID NO: 1875); ZC3H13, Del, A_7, 41, essential, high expression, KKDRKRKALKKNVKNPKVILIFLMKKQPSKVRRKEAHGLPL (SEQ ID NO: 1876); PCNT, In, A_7, 41, essential, high expression, KKNGSRAAVPVCGRPAERQCFPHKSAQHGDPGEAGAEQSRV (SEQ ID NO: 1877); DYNC1I2, In, A_7, 41, essential, high expression, KKNYPRVCVSLPVSCDVCHICKISSKSCCWWYIFRPNCALG (SEQ ID NO: 1878); LAP3, In, A_7, 41, high expression, KKEDGCVGKALWKWGSGGLAERSPVCFWAELGTPIDGDASQ (SEQ ID NO: 1879); TRAF3IP1, Del, A_7, 41, high expression, KKFWRRRKIMRNCSSHPNLGRRSDLSLSRHGRRRRTSFPRR (SEQ ID NO: 1880); LYN, In, A_7, 41, high expression, KKRRLHPQQLCGQTQHLRNRRVVFQGYNQEGRRKAAFGTRK (SEQ ID NO: 1881); ODF2, In, A_7, 41, high expression, KKTPETISDQQPEISQARNERGHCECAAECPGENQGQDAKR (SEQ ID NO: 1882); ZNF326, Del, A_7, 41, high expression, KTVRNTEMDTEWHLHVHFVNFEHLKKKILNCIWKVLHIRKH (SEQ ID NO: 1883); BOD1L1, Del, A_7, 41, essential, KKIQGMLKRTPKRNSNMKKIPKKPLKQVSIVKRKKFLLQRS (SEQ ID NO: 1884); BARD1, Del, A_7, 41, essential, KKMQVLSKTHMNLFPQVLLQMFLRGLKRLLQDLEKSKKRKL (SEQ ID NO: 1885); CEP192, Del, A_7, 41, essential, KKQKLVDMKVHWKTFQGLVCLILGIYLCPKNKLLKTFIRWT (SEQ ID NO: 1886); POLR3A, Del, A_7, 41, essential, KNSLRGSLRRSRKPEINMASMITAQQSPVCCTSWTASPPPK (SEQ ID NO: 1887); PRRX1, Del, A_7, 42, driver, high expression, KRRESSEGIGQPSIAASCRLWSVSLSGHTILMLLCEKTLPAG (SEQ ID NO: 1888); NOSTRIN, In, A_7, 42, high expression, KKHGGLFYQTEMGKHTRELLPGNPQHLPICLLGQKSLGKVCE (SEQ ID NO: 1889); UBXN4, Del, A_7, 42, high expression, KNLKKGEKRKEKRKNREKLRRKLRGEKLEKKCWIIKENKKKN (SEQ ID NO: 1891); ZNF880, Del, A_7, 42, high expression, KNMREFILGRNRTDVMNVVRTSLEIQTWQIITESILERNRTD (SEQ ID NO: 1892); NIPBL, Del, A_7, 43, essential, high expression, KMKGDLKHQNTGMTIGGILESHLQRKNLKCLNINKILNLTHLG (SEQ ID NO: 1893); NKTR, Del, A_7, 43, high expression, KIRKVIRALIQNTVIEVQKAHQGQGADLLGVDLIPDHIQDHVV (SEQ ID NO: 1894); CHD3, In, A_7, 43, high expression, KKGEEDKAAEKGGGRWGAKASGTEVISNSASDLGPGGCGACVL (SEQ ID NO: 1895); KIF21A, Del, A_7, 43, high expression, KKGYRNLRKAIEKKEVWLVKRIIQTLTKRRKKKRVFRKEKTMN (SEQ ID NO: 1896); RYR2, In, A_7, 43, high expression, KKNEATQELPADKWIQACPYGPELYQTHPITRSNGGQVGRKCT (SEQ ID NO: 1897); IFI16, In, A_7, 43, high expression, KKNNVSCYSGYTDTVLPCEGFKHQLEGEIQWKENHHHIRLFGI (SEQ ID NO: 1898); UTP20, In, A_7, 43, essential, KKSSSPSTRSTSTTPELPSASPNSSSRWTESCCEQENQHAHIY (SEQ ID NO: 1901); TRIO, Del, A_7, 43, essential, KNVLLLWIRGTEISLCGWRSTGPLWKKPWGFLQIPTNRVKVSS (SEQ ID NO: 1902); PTCH1, In, A_7, 43, driver, KKLRQVLGCGPPHIWGLRGGIKSSEPRDQRGGAVGGSWRTSKS (SEQ ID NO: 1903); PRPF38B, Del, A_7, 44, essential, high expression, KNGNIVPAKKNLESVVEAKNVPTNEITVIVRTSQTNMIVEGAKV (SEQ ID NO: 1904); PSMD1, Del, A_7, 44, essential, high expression, KRRRRKWKWMRQRKRRKKRRKKNLSQTSSYWITQPELCLPSLRS (SEQ ID NO: 1905); TXLNB, Del, A_7, 44, high expression, KKSETQKYLKRMTKVSTTPMKSQSQTSLWIKRLTQRRLIVSKPP (SEQ ID NO: 1906); BMPR2, Del, A_7, 44, high expression, KTEIQLTMNDSKHKLESPALKQVSPASPPTQQPQTPQDSRQVLA (SEQ ID NO: 1907); NSD1, Del, A_7, 44, driver, KKCRVNAVEELHSRRMSVRIVKNWVSCCYVRLSAVGLSTWSALD (SEQ ID NO: 1909); ATRX, Del, A_7, 45, driver, essential, high expression, KKELENKMKKTQEMRKQKIKSILNQIQILKNLRSQDTDIGFCGTN (SEQ ID NO: 1910); NSF, Del, A_7, 45, essential, high expression, KKALTPTLMTPTRWQQNLFSNSTTRPSQWDNSLSLASMKSFLAYW (SEQ ID NO: 1911); SNW1, Del, A_7, 45, essential, high expression, KKCRMRWPFRWILKEKLNMMQLLDKDSQKTRSFIANTLTWFQRRL (SEQ ID NO: 1912); SECISBP2L, Del, A_7, 45, high expression, KIRHFLEVEGKLNKEIIHRLDSDAEDTVLPQKEDRICKRDQIISI (SEQ ID NO: 1913); TOP2B, Del, A_7, 45, high expression, KKKSQKLPTKMILQRSCLLRECIRRRHNLNTFFFVLIHILGQWSH (SEQ ID NO: 1915); BICC1, In, A_7, 45, high expression, KKNLCCFKWTCTVSRYKIWCNIHFITWRKSAECKSRGSVHPDKWV (SEQ ID NO: 1916); HP1BP3, In, A_7, 45, high expression, KKNPAEMRKEWVDGTDLWERVQWHLPALFSLLSQPRSSVSEERAR (SEQ ID NO: 1917); ANK3, Del, A_7, 45, high expression, KRCSPSCPMRKKVPQETRRCPRLPGVASLRLQRSLLEIRKQRQHL (SEQ ID NO: 1918); SMG1, Del, A_7, 45, essential, KILLHQLILHQAPFQELARVLLVVLKRQSETLKLGKRCKRETPMQ (SEQ ID NO: 1919); PLAG1, Del, A_7, 45, driver, KSTSANIVIAGSTPERMSGDTWWCTLEERTSSVSIVHRDLGERIT (SEQ ID NO: 1920); ZNF131, Del, A_7, 46, essential, high expression, KKFMYVSTVRNSLTILDILKNIFENIQVKNLLNVQIVMNDLLEIAL (SEQ ID NO: 1921); ATM, Del, A_7, 46, driver, high expression, KSLLKRNMKSSWMFAKIFNQFSVTSAWKNSWIQLFGLRSDWLIRAV (SEQ ID NO: 1922); MGA, In, A_7, 46, high expression, KKRPGHRKYLTLEHSTHLCQPCDDSARAHHPKRSPILRTSGSCFS (SEQ ID NO: 1923); CTSH, In, A_7, 46, high expression, KKWDPLLDREKLLGSPVGNERVLPHRARKEHVWPGCLRLLPHPSGV (SEQ ID NO: 1924); INO80, Del, A_7, 46, essential, KKDEEMKNFPLKNPLVAITTRPKSLPSFLTMHLPLALRKSTYPLSS (SEQ ID NO: 1925); NUP153, Del, A_7, 46, essential, KNQHSTCLPLEHFPLHLGILQSLKPVSLEILLFILEKQHTVGQQLL (SEQ ID NO: 1926); RPS8, Del, A_7, 47, essential, high expression, KKFRRNMMKGKRMPKSAVSWRSSSSRASFLRASLQGRDSVAEQMAMC (SEQ ID NO: 1927); WAPL, Del, A_7, 47, essential, KKELKQLHHPPCSLPQKAMIIPRTVSLVLTMQKTWILQRTCLVCLKV (SEQ ID NO: 1929); POLA1, In, A_7, 47, essential, KKKPTSCTCCPLDKFSRRQKGESWRYCVICHLSGWIKPHCKSEGLCA (SEQ ID NO: 1930); RBBP6, Del, A_7, 48, essential, high expression, KKSNSTEKLGRKLEVQKIYQTQKNPLKNWSQHLAKLNKKKSKERSDEK (SEQ ID NO: 1931); ASCC3, Del, A_7, 48, essential, KNKSLVKKPNYWYLQSLFLSLCLPNTTSEQCLIDGWVLRQYVLSTFNI (SEQ ID NO: 1934); CWF19L2, Del, A_7, 49, essential, high expression, KKQRKKRKKRARNRNMKKTMSHLHHQALKMSGLRLFHPRLLTRKKPGK (SEQ ID NO: 1935); HMGNS, Del, A_7, 49, high expression, KKVKTEKEMEKMEKRKEKMKKRKKTEKKQEMEKRMKMEKRREIKKRGKM (SEQ ID NO: 1936); LUC7L2, Del, A_7, 49, high expression, KKYREHRREWEIQLVNESNSVMTEYARVTFSTVVLMMSFLELEWILENV (SEQ ID NO: 1937); BTK, Del, A_7, 49, driver, KILLQKDRFREEVKSPVKWSKFQSLKGSLIPSRLYMMKGLSTSSPQLKN (SEQ ID NO: 1938); PSMA1, Del, A_7, 50, essential, high expression, KKFSMLTTILVSQLRGLLLMLDCYVILCVRSVWIPDLYSIDHCLCLVLYL (SEQ ID NO: 1939); ATP9A, In, A_7, 50, high expression, KKRVMLLADGSAGWGDGLEAAASRGLHAEAPHGRRPSSDSIVCVRRRAKY (SEQ ID NO: 1940); IBTK, Del, A_7, 50, high expression, KSLLLAIVQAIMSKKFLLKELKILRHQKLSDALPMVPQDQKATIFQIYHF (SEQ ID NO: 1941); SPEN, In, A_7, 51, driver, essential, high expression, KKLQIKERKISKLQVSSGQICKSEKCGCCCQSQGGCSTGRGEGIWGGGSLP (SEQ ID NO: 1942); FAM13B, Del, A_7, 51, high expression, KRNYAKRCGNLKKHFINKMEGMPRKRIVFQCLRSTESTRKLKPSLGFLKFL (SEQ ID NO: 1943); UPF2, In, A_7, 52, essential, high expression, KKETSRGREKEARRAGQTSARRRSSCSDERKRRIHSASSGSLGTTSFKKGTS (SEQ ID NO: 1944); CBLL1, Del, A_7, 52, essential, high expression, KREIRCVQAVVILCSELSSVHEVLSSCVALFKGAREHICLRETYRLISTIAI (SEQ ID NO: 1945); ZNF141, Del, A_7, 52, high expression, KKFILERNPTNVKNVAKPLDGPQIGVNIRKFIVQINPTNVKNVTKPLNNFRS (SEQ ID NO: 1946); MYO1B, In, A_7, 52, high expression, KKKPNCDCRLVQEICATKEVPADKEFRLSNSVLYPGLEGSKNSAGTEASKAL (SEQ ID NO: 1947); EIF2A, In, A_7, 52, high expression, KKNQEPKEETESNRTTERTSSNWKTARKKSVGENSERNSPSPGAGRFGIGYL (SEQ ID NO: 1948); ZMYM4, Del, A_7, 52, high expression, KKSSRRGKLLIRGKGLLSYSAPHCASLDIQFHLPAHRLLSPRKLVQVAQKTF (SEQ ID NO: 1949); GIGYF2, Del, A_7, 52, high expression, KKYRKSLFQKSRRWTSGLWTKGRSALTLRVAIMKRPKNPIRQIRKKERKQIE (SEQ ID NO: 1950); RSPO3, Del, A_7, 52, driver, KNLIKEKVKKQYLTAKVWNPAKKSQSNEKTNSSRRSEKSKINRNRYQSALYT (SEQ ID NO: 1951); EPAS1, In, A_7, 53, driver, high expression, KKKQRHVHRAGLLHEDEVHGHQQRPYCQPQVSHLEGLALHGPGESLQQLPSSQ (SEQ ID NO: 1952); BTN3A1, In, A_7, 53, high expression, KKDSVQKEKERARVERNGMEHNEARTKHKSEAPGGTQMEKYPVCISGRETFSL (SEQ ID NO: 1954); KNG1, In, A_7, 53, high expression, KKNLPYCQLSTTGNDLTDEKASRFFTFPIITNRGNKRRNNCKSTPHFHGTCTR (SEQ ID NO: 1955); CD46, Del, A_7, 53, high expression, KNFTTKQQLCLNAIRVFTSMAATQLSVTVTVLGIPQFQSVLKCCLHLVQNLQL (SEQ ID NO: 1956); ZFC3H1, Del, A_7, 53, essential, KILRMQRFFIICANSSSYRIEAIIFFHFCGNLLHPSLNRGLRSIITWICFGIS (SEQ ID NO: 1958); CAPZA2, In, A_7, 54, high expression, KKNRWTANHYCMHRKPSVPSKKFLEWSLEVRMEVYNHSFNHSSGWHLENSGSLL (SEQ ID NO: 1959); HIST1H1D, In, A_7, 54, high expression, KKQQPYQAWPQELGEQRYSGADQRYRCFWLLQTQQESGFRGRQTQGQKGWRSQA (SEQ ID NO: 1960); ZFAND6, Del, A_7, 54, high expression, KRIAVSCAGRKWDLLGLNAGVEMFTVVYTVTQMYTIALTITKPMLLRKSEKKIQ (SEQ ID NO: 1961); BOD1L1, Del, A_7, 54, essential, KINQQTKVKRSQTAMRKEKERKKRRKRLKRNLITQKRVKIHRKLKMKNKQRKKK (SEQ ID NO: 1962); EML4, Del, A_7, 55, driver, KRKKNHKDREKKKRNLILMIKVHKFEHHLLPSPLHNLSKYTDKLQKARMLLPPKA (SEQ ID NO: 1964); PLIN2, Del, A_7, 56, high expression, KKLKDLIWFRSQVIMLDWDPCLPSFTPVPTSRLSAGLKKLSKKANRPFLSSILLFT (SEQ ID NO: 1966); ABL2, In, A_7, 56, driver, KKIRGKCCSKQGETKSQVIAQRSHSSSSQNTLWGSSHYREGPSRGGSGWSGSCPQG (SEQ ID NO: 1967); KIAA0100, Del, A_7, 57, essential, high expression, KKEPSPKSWLPSSLAPPSPMSTFPFNLEIHHLLPWDSILSLWITSTSGHKASISGAS (SEQ ID NO: 1968); ZNF138, Del, A_7, 57, high expression, KKFILERISTNVKSVEKPLTGPQTFLNLRKFILEKNPTNVKYVEKPFTNPQSLLNIR (SEQ ID NO: 1970); PNISR, Del, A_7, 57, essential, KRPQKMLKEGMALVYLREVNLIVMRKKKTLKMLRLQVVGKSPEVHPQFLKKSTVTLR (SEQ ID NO: 1971); GRIN2A, Del, A_7, 57, driver, KKAPFSPKVWRTARGASLSCQTTPPITLSSTPTGMTNAWLLGDAPRTLTNTRCHPRR (SEQ ID NO: 1972); TAF7, Del, A_7, 58, high expression, KKLFTRQLISVRCLYPQLMVISILLWRSQLLALILKQARKRIRTKRKSLSGTTELLCL (SEQ ID NO: 1973); CREB3L3, Del, A_7, 58, high expression, KKSAGKSGTSSRRKKAGRRRRNISMAWRLGCQLALLRIRSYRGKSCISRSKTCPSWSN (SEQ ID NO: 1974); TACC3, In, A_7, 58, essential, KKYRKLRLPVFATRSYRKIVCSSCVTERKCATQEPGQSYEGDFSDTSAGSTDAQDSKS (SEQ ID NO: 1976); ZNF7, Del, A_7, 59, high expression, KSYLTACRGNIQITAMERSRTNVQSVGKSSGSARSLISIRESTRERNPLNALSVEKPSA (SEQ ID NO: 1977); KIF5B, Del, A_7, 60, driver, high expression, KIRSCGTLFSGLKMSSTDGVMGRRCLLMNSLTKRKPTWKLSQWIKILLLPMINQQPQLEL (SEQ ID NO: 1978); ATF6, In, A_7, 60, high expression, KKNSGEFKTFNSAQAFIASSSTQDSNKLQCSSKNHHYSDSTNAYAIGKAATNYQFTTCTH (SEQ ID NO: 1979); SLC39A8, In, A_7, 60, high expression, KKRAKFMYLFEGAQTVRNRDDCLDDNALRCPPQFHRWPGDWGFLHLVSPSGTQYFHSNPM (SEQ ID NO: 1980); SFSWAP, Del, A_7, 60, essential, KKDLEQDHVLPSTIRHPSPGLDHTQKQSILFPVPIGQCGGRESQPPQGRCARSPVRAARQ (SEQ ID NO: 1982); RSPO3, Del, A_7, 60, driver, KKEGRGKEKNLIKEKVKKQYLTAKVWNPAKKSQSNEKTNSSRRSEKSKINRNRYQSALYT (SEQ ID NO: 1983); MPHOSPH8, Del, A_7, 61, high expression, KKKNLSNPRWNLNQVYLMILPFQRMTVKGYIPTAEKRNKTLKVQEREQGRTWGWSMALRSP (SEQ ID NO: 1984); C1orf131, In, A_7, 61, essential, KKGSKGRRKETGPRNRYFQEKEEERTGGQEIQKEVRSQYFVKWTDWTGWKIQKWNTDSEPS (SEQ ID NO: 1986); CACNA1D, Del, A_7, 61, driver, KKCSINGSSKRTTRTRQTMQEAPDFLFLVKDQLLSRIAPSKLSCLGKLQSMLLDRPRLPKL (SEQ ID NO: 1987); TCF12, Del, A_7, 63, driver, high expression, KKSERCLLVCLLLYMHHPQIQMISTVNLLVIHLLSHQPVCSLALSLCKMGPTILLTFGVHQMG (SEQ ID NO: 1988); ZNF280D, Del, A_7, 63, high expression, KKKLPQRNKNLCLRKLQDLTWLKEKQKHQILKVNKIKLLLQKKKMDVMQIHLKAHQQQKVKKA (SEQ ID NO: 1989); ZMYM4, In, A_7, 63, high expression, KKNPPEGANCLSEERVYSAILLHTVPHWIYSSTCPPTASSHQENLFKLLKRHFKSKGCDQCPV (SEQ ID NO: 1991); IFI16, Del, A_7, 63, high expression, KNQPKKRLDPKGVRCPRNRLSLPLLQEPACPQPWAVPHLPRPHCQLHPTVLQLRTRKQWPNVR (SEQ ID NO: 1992); MYH10, In, A_7, 64, high expression, KKNASTYSGPGRTARRGGRGSAKAAAGKGDSRGQDQEDGRGDSASRGPKFQVHQRKETHGRSHC (SEQ ID NO: 1995); CAPN3, Del, A_7, 64, high expression, KKPSPSSSFRTEQTATRSWVWTRSQRRAKAKQALISKSSPHSHSLAALIRKVRNSNNSGTFSSR (SEQ ID NO: 1996); SEC62, Del, A_7, 65, essential, high expression, KKKKRKKKMVKRKNPKRRKLQELLKRRKLRKNSNLSHMMIRFFWMEMRCMYGSMTQFTLKHLS WD (SEQ ID NO: 1997); ENDOD1, Del, A_7, 65, high expression, KKSWKWLTKSRMKNEWYNLKRVLVPFLAPGARGLLCCLQRHLREVVAFWENSWASLLPHSSSFF N (SEQ ID NO: 1998); CFI, Del, A_7, 65, high expression, KNILTSPAIKSSASHGRDALRAPVFVNYRISAQRMALQCVQLTGEASQHTVNKRVWNVFIQGQSF (SEQ ID NO: 1999); KLC1, Del, A_7, 65, high expression, KNMMTTFPHPRTKTLILPKSLWMTFSPMMKTTQGKESSSSTAVQPRLPSRAATRSPRGCGRSTTW (SEQ ID NO: 2000); BOD1L1, Del, A_7, 65, essential, KNFHLSQPLILVLTKKELQRTWLIKKNLQLTLEVKDWKQPQSLKSSATSPVQSKKLKITQKSIII (SEQ ID NO: 2002); PHF20L1, Del, A_7, 66, high expression, KRKTTMKQLQCWSRRFHLNLKVRKKMKLTLAVLPTLRNLHCYPQLCLQGRLAARNANMNLEILL GV (SEQ ID NO: 2003); TCF25, Del, A_7, 67, high expression, KKASPSLRLSTVRSTSRLSTSSWWPWSLWSRTTSWFCSRRALTTLTHSCSSAMPAAFKRIRRWLETS (SEQ ID NO: 2004); NKTR, Del, A_7, 67, high expression, KKGRNQLIQKARIPLPIPPLLQNHLQKVNLNMREAEGGNIRGGQKLNVLKRGERKQAVQKSQGIN MQ (SEQ ID NO: 2005); MYO9A, Del, A_7, 67, high expression, KTNMIHLILPGMAELGFARADYQVAPPCLIKMEYLLIQLAANSWREPMEFSRETKISNPSLPFQSTC (SEQ ID NO: 2006); RPL32, In, A_7, 68, essential, high expression, KKNKAHAAQWLPEVPGPQRQGAGSAADVQQILLCRDRSQCFLQEPQSHRGKSCPTGHQSHQPQCQ AAQ (SEQ ID NO: 2007); GOLIM4, Del, A_7, 68, high expression, KKGNWSIMLKRPMVKMMKILMIKIMMEKSKKFEMTTAPKAERNTTRRKKRRKKTGLQLLRNHIE ELKC (SEQ ID NO: 2008); NIPBL, Del, A_7, 69, essential, high expression, KKLTRMLSLLTLMKQLCEQPKTSSPSSLKNVVVSKVKKITDHCLKILFKTFFQQSISLNGQLLNYSL VC (SEQ ID NO: 2009); SFSWAP, Del, A_7, 69, essential, KKDLEQDHVLPSTIRHPSPGLDHTQKQSILFPVPIGQCGGRGPAPGPLGGEPTPLRDGGKRGVCPLPT A (SEQ ID NO: 2010); NRDE2, Del, A_7, 70, essential, KKGSISIIRKQRGSMGRRVAAGLRQTPILKRTNLPEALEAVKRNLRNRIKEIMLQLILDIALFGLRTFR L (SEQ ID NO: 2011); BRE, Del, A_7, 71, high expression, KIIRTAPDGMEMKWPKEQRDAKGAEMPAARGSKSWPLQLQKLAASCSSPRGTGQAPEGLLGGPQR ERELLS (SEQ ID NO: 2013); ATP9A, Del, A_7, 71, high expression, KTGHASCGRISWMGRRTGSCGFPWPARRGSPRPPTFFRFDRMCTQKSQILTFTTSWELLPEKTATPR SARA (SEQ ID NO: 2014); KLF3, In, A_7, 73, high expression, KKILTPFSRRAALRRAAVHATSDGSCPLAAWNTEPGDPARHPAGGGAARPLYVHKSPPAASHGLLI GGDGKFQ (SEQ ID NO: 2016); TNKS2, Del, A_7, 75, high expression, KNCVLFRVSTAETLKGVSLHHFILQLGITECPWWNICYSMELMCMLKIKEALYLCTMHVLMDIMK LQNFLLNMEQ (SEQ ID NO: 2017); CD46, Del, A_7, 75, high expression, KNFTTKQQLCLNAIRVFTSMAATQLSVTVTVLGIPQFQSVLKVLGLLTSLQSQIIQDILNLRKEYLTV WMFGSLL (SEQ ID NO: 2018); DENND4A, Del, A_7, 76, high expression, KKCSQRRWIHLMRYATAFLCNSVDNMISQCLQFECFLKCRKLVLTPMPLLMVIIIRLFWKVPGLQE VVVAIFFGQK (SEQ ID NO: 2019); ODF2, In, A_7, 77, high expression, KKTPETISDQQPEISQARNERGHCECAAECPGENQGTLDAPWKIICPACGMQVGESTSLPGDHATIF RKAGLSDAVK (SEQ ID NO: 2021); WDR33, Del, A_7, 77, essential, KKHKQKLSKKWLHYNILTHNFWSNLKLKDLHRNKLSKFSLLPHLAPLSSDPSLFQDKVQCLRFLKV FNSPIHLSRCQ (SEQ ID NO: 2022); TRRAP, In, A_7, 78, driver, essential, KKNTRGHVLSSLGRRAAREHGQPATGVPGSESRHRHHDPPAQPRPVRRRGKQGEHPGGRGKQPGQ SVPHGPRLAPLAV (SEQ ID NO: 2023); RBM39, Del, A_7, 80, essential, high expression, KKARAEVVVMNEREAKVRNGSEVETEKGKRAKAVKESEVEAKRGDGAAQEVEIEDLEAATEVLT DDVPEAKVHSEKTRAL (SEQ ID NO: 2024); PPAT, Del, A_7, 80, essential, KKHQKQKDGWCLQNLVASYLLVQDITVKSCLEKLWKYPDTMSKLLILYQGLKETQWLFVSLNMFI LQDQTVCSKTKWFIQ (SEQ ID NO: 2026); RBM39, Del, A_7, 84, essential, high expression, KKARAEVVVMNEREAKVRNGSEVETEKGKRAKAVKESEVEAKRGDGAAQEVEIEDLEAATEVLT PDQNLTVPSEERLGCLIASN (SEQ ID NO: 2028); TLN1, Del, A_7, 85, essential, high expression, KKARITLGWKEMRSLLCWRTQCPPKSQQSCSSNTTGWGKWSMALWPCLPSCALEPLVLRISRWAA CPLPSSRLPAARCTEDTCLL (SEQ ID NO: 2029); MY018A, In, A_7, 85, high expression, KKNHQQPVSGPRRQCHGALWLHRGPGGRLAAGTAPGHQHAENLYHRHGGCQKEVTVHPDEATG GRPHRHHQEVKAAFCALLPACS (SEQ ID NO: 2030); CCAR1, Del, A_7, 86, high expression, KKAKKMREKIKKKKEMMKLMNQNPNGENQAMIKIKKKIEMKGRKKIKEKMILKMMMKLKKITIK MNMTLWKQKKLRMKKMIGMRKK (SEQ ID NO: 2031); PNISR, Del, A_7, 86, essential, KKSIKKKKNKEGVGREVLVVVVPVAIAELVVLVVLSLALHTVLAQVVVVLLLGLLLLKGKRDTVG VDLQQSKLDVAGVEAILAELK (SEQ ID NO: 2032); HP1BP3, Del, A_7, 87, high expression, KKPCRNAKRMGGWNRSLGKGSVAPSSSVFPIIPAQEFCFRRKSQMILEMRMKMKMSHQKKTLRM KSRHLREGCRRKPQPSPQGRPHL (SEQ ID NO: 2034); RYR1, Del, A_7, 90, high expression, KKRGRYHKVPRPMILEKATTLSPPTLVLLPCPGSCRPWQNNWQKITTTRGDGRRSRSWKPKAVGPT PCWSPTTRSRPRRRHHERRPRSY (SEQ ID NO: 2036); LIMCH1, Del, A_7, 90, high expression, KKWRSYWLEKMGQVNEGKASKPTEKLFKKKSGERESCMKHIRTLGPRRRQRGSFNSTLRGSPSVR LFSNAWRCQKFWKEAIQQSQIYPPS (SEQ ID NO: 2037); CD46, Del, A_7, 90, high expression, KNFTTKQQLCLNAIRVFTSMAATQLSVTVTVLGIPQFQSVLKCRLLPLQNLQRPVPQVLGLLTSLQS QIIQDILNLRKEYLTVWMFGSLL (SEQ ID NO: 2038); NARFL, In, A_7, 92, essential, KKNRGALRLRHRLLKALQPPGEPARVCAAIPRTGRLQTGAAPAGLCLPRLDLLCREDSRQLHPPPH QHRPVPAAGHGLPGQGLLRPAAALDP (SEQ ID NO: 2039); PRPF40A, Del, A_7, 94, essential, high expression, KKDSDQSLVLLQNILLVQSLREVIKSQKSIRRKVRRGDINLTLQNPMLSERRIKKKKIGKVKKTELD KDQNQNTNRLRKRLERILVIGILLAAN (SEQ ID NO: 2040); CKAP5, In, A_7, 95, essential, high expression, KKMAREKRGPGVCRSTNKKPQTGSWRLCRFSKSIKEGCWKGHQCHVGGFGSKMSYWPGCWAKE EIWTICRTCCANHLGEIQREETSSGTSPAGGN (SEQ ID NO: 2041); BRAF, Del, C_7, 8, driver, essential, high expression, PLPHYLAH (SEQ ID NO: 2042); MBD2, Del, C_7, 8, high expression, PPDGRRRK (SEQ ID NO: 2043); ATP2B4, Del, C_7, 8, high expression, PPKSPRLS (SEQ ID NO: 2044); BFAR, In, C_7, 8, high expression, PPPESLGI (SEQ ID NO: 2045); PPPSC, In, C_7, 8, high expression, PPPGRTPG (SEQ ID NO: 2046); FNDC3B, Del, C_7, 8, high expression, PPPSKHLE (SEQ ID NO: 2047); APOA1, Del, C_7, 8, high expression, PPRAPGIE (SEQ ID NO: 2048); MARCKS, In, C_7, 8, high expression, PPSGGGRV (SEQ ID NO: 2049); ARFGEF1, In, C_7, 8, high expression, PPTSISCK (SEQ ID NO: 2050); PIP4K2A, Del, C_7, 8, high expression, PQIAPGIH (SEQ ID NO: 2051); BRD4, In, C_7, 8, driver, essential, PPPSAAGG (SEQ ID NO: 2052); KMT2A, Del, C_7, 8, driver, essential, PRLLSLPR (SEQ ID NO: 2053); UBR4, In, C_7, 8, essential, PPHAGHPT (SEQ ID NO: 2054); PTPN23, In, C_7, 8, essential, PPPFLPTT (SEQ ID NO: 2055); NAB2, In, C_7, 8, driver, PPCRGRSP (SEQ ID NO: 2056); CBFA2T3, Del, C_7, 8, driver, PPPSRHTH (SEQ ID NO: 2057); ANKRD11, In, C_7, 9, essential, high expression, PPRRVPLCP (SEQ ID NO: 2058); SIPA1L1, Del, C_7, 9, high expression, PPEVKVLTI (SEQ ID NO: 2059); TSPAN7, In, C_7, 9, high expression, PPQLLHERN (SEQ ID NO: 2060); MAP1B, In, C_7, 9, high expression, PPRSEWLQL (SEQ ID NO: 2061); RYR1, In, C_7, 9, high expression, PPTLHSCHL (SEQ ID NO: 2062); TGFBI, Del, C_7, 9, high expression, PQWGLSWMS (SEQ ID NO: 2063); TNPO3, In, C_7, 9, essential, PPLYPTRCG (SEQ ID NO: 2064); POLR3E, In, C_7, 9, essential, PPPDCCFPG (SEQ ID NO: 2065); NCOR2, In, C_7, 9, driver, PPHPGHQSP (SEQ ID NO: 2066); AFF3, In, C_7, 9, driver, PPPLLQPRG (SEQ ID NO: 2067); NFATC2, In, C_7, 9, driver, PPQDVEDQP (SEQ ID NO: 2068); BRAF, In, C_7, 10, driver, essential, high expression, PPCLITWLTN (SEQ ID NO: 2069); NRBP1, In, C_7, 10, essential, high expression, PPHHPWEPDL (SEQ ID NO: 2070); AAMP, In, C_7, 10, essential, high expression, PPTGDPKLPW (SEQ ID NO: 2071); ATP2A1, In, C_7, 10, high expression, PPEARGNGPG (SEQ ID NO: 2072); NES, Del, C_7, 10, high expression, PPHLRHPLLL (SEQ ID NO: 2073); RCSD1, In, C_7, 10, high expression, PPQGRPGPEW (SEQ ID NO: 2074); BFAR, Del, C_7, 10, high expression, PPRISGNIRL (SEQ ID NO: 2075); ADM, Del, C_7, 10, high expression, PRVEVLPTFF (SEQ ID NO: 2076); MCM3AP, Del, C_7, 10, essential, PPGFLLHQRR (SEQ ID NO: 2077); THOC1, Del, C_7, 10, essential, PPMEKDFQRW (SEQ ID NO: 2078); EXOC2, In, C_7, 10, essential, PPPCDRHLSK (SEQ ID NO: 2079); SLC16A1, Del, C_7, 10, essential, PQMEAGAGQW (SEQ ID NO: 2080); MYCN, In, C_7, 10, driver, PPGGGHLEEV (SEQ ID NO: 2081); MKL1, In, C_7, 10, driver, PPSTQPLHYQ (SEQ ID NO: 2082); NF1, Del, C_7, 10, driver, PRFADKPRPN (SEQ ID NO: 2083); USP9X, In, C_7, 11, essential, high expression, PPPPTESDFIA (SEQ ID NO: 2084); TNFAIP3, In, C_7, 11, driver, high expression, PPQAALPGPRL (SEQ ID NO: 2085); SYNPO2L, Del, C_7, 11, high expression, PLQWLPRPRPL (SEQ ID NO: 2086); ADM, In, C_7, 11, high expression, PPEWKCSPLSL (SEQ ID NO: 2087); PPP5C, Del, C_7, 11, high expression, PPGTNPRLMEL (SEQ ID NO: 2088); SORBS2, In, C_7, 11, high expression, PPRSGPARLPV (SEQ ID NO: 2089); TACC2, In, C_7, 11, high expression, PPSEGDATGAR (SEQ ID NO: 2090); UHRF1BP1L, Del, C_7, 11, high expression, PQLEHLNHSHL (SEQ ID NO: 2091); SYVN1, In, C_7, 11, essential, PPPACHFSQLH (SEQ ID NO: 2092); NCAPD3, Del, C_7, 11, essential, PPRSEVLSCPL (SEQ ID NO: 2093); SNAPC2, Del, C_7, 11, essential, PQPPRRPSTAN (SEQ ID NO: 2095); RCC1, Del, C_7, 11, essential, PQQMPSPKARR (SEQ ID NO: 2096); EIF3G, In, C_7, 12, essential, high expression, PPRTQCGHHHCQ (SEQ ID NO: 2097); HLA-A, In, C_7, 12, driver, high expression, PPQDTYDPPPHL (SEQ ID NO: 2098); HSPB8, Del, C_7, 12, high expression, PHPSLGSPGKCV (SEQ ID NO: 2099); TF, In, C_7, 12, high expression, PPQDGCQDVPGL (SEQ ID NO: 2100); SERPINA1, Del, C_7, 12, high expression, PPRSSSTNPLSS (SEQ ID NO: 2101); SSH2, Del, C_7, 12, high expression, PQNIHLSSRNQK (SEQ ID NO: 2102); TIMP3, Del, C_7, 12, high expression, PRIKASSMPQTP (SEQ ID NO: 2103); NCAPD2, In, C_7, 12, essential, PPLPAASGTQVV (SEQ ID NO: 2105); MCM3AP, In, C_7, 12, essential, PPPASCYIRGAE (SEQ ID NO: 2106); BAG6, In, C_7, 12, essential, PPWYSGAWGLCS (SEQ ID NO: 2108); TCF20, In, C_7, 13, high expression, PPPGEQLWQSVWE (SEQ ID NO: 2112); ITM2B, In, C_7, 13, high expression, PPRRRRGGLQGPR (SEQ ID NO: 2113); EPHX1, Del, C_7, 13, high expression, PPSCPQAIPRSPC (SEQ ID NO: 2114); TNS1, Del, C_7, 13, high expression, PRVWPRHPCLLWA (SEQ ID NO: 2115); POLE, In, C_7, 13, driver, essential, PPLVPRGASSGLS (SEQ ID NO: 2116); KMT2A, In, C_7, 13, driver, essential, PPVFCLFLVKHFC (SEQ ID NO: 2117); NCAPD3, In, C_7, 13, essential, PPPGQRFCHALCD (SEQ ID NO: 2118); CIC, Del, C_7, 13, driver, PHPQVPPQLPQAG (SEQ ID NO: 2119); NCOR2, Del, C_7, 13, driver, PPMRRPPPSAPMT (SEQ ID NO: 2120); MAZ, Del, C_7, 14, high expression, PLSRETPPSHSPPA (SEQ ID NO: 2122); EGR1, In, C_7, 14, high expression, PPPRTPLRLPSGVL (SEQ ID NO: 2123); SYNPO2L, In, C_7, 14, high expression, PPSNGSQDPAPYDS (SEQ ID NO: 2124); C3, Del, C_7, 14, high expression, PPSPRCPFHMSSCR (SEQ ID NO: 2125); SSBP4, Del, C_7, 14, essential, PPAPSRPPTTPTPP (SEQ ID NO: 2126); TARBP2, In, C_7, 14, essential, PPHGTAAPCLPSAV (SEQ ID NO: 2127); JAK3, In, C_7, 14, driver, PPPAPIFLLWGPLG (SEQ ID NO: 2128); TCF7L2, Del, C_7, 14, driver, PPQTSLSALKSSGT (SEQ ID NO: 2129); CIC, In, C_7, 14, driver, PPTPRSLHSCHRQV (SEQ ID NO: 2130); WBP11, Del, C_7, 15, essential, high expression, PLGCSHQLPCQTLGF (SEQ ID NO: 2131); PELP1, Del, C_7, 15, essential, high expression, PPCHPHHPQVPHHPL (SEQ ID NO: 2132); EIF4B, In, C_7, 15, essential, high expression, PPPKTQTESKASEYS (SEQ ID NO: 2133); MAZ, In, C_7, 15, high expression, PPCPGRRPPATAHLL (SEQ ID NO: 2134); MAP1A, In, C_7, 15, high expression, PPPCSYPEQRPKPPS (SEQ ID NO: 2135); SERPINA1, In, C_7, 15, high expression, PPRGQVQQTLCLLND (SEQ ID NO: 2136); NFE2L1, In, C_7, 15, high expression, PPSQWRLNQRGHRSD (SEQ ID NO: 2137); EIF4G2, Del, C_7, 16, essential, high expression, PPAMKQIHPLLLPKNS (SEQ ID NO: 2138); EIF3G, Del, C_7, 16, essential, high expression, PPDPMWPPPLSVTMSL (SEQ ID NO: 2139); SETD7, Del, C_7, 16, high expression, PPGRVGLKPLSGTRWS (SEQ ID NO: 2140); TGFBI, In, C_7, 16, high expression, PPNGDCHGCPEGRQSL (SEQ ID NO: 2141); GRINA, In, C_7, 16, high expression, PPQPLWTATGLPRTRP (SEQ ID NO: 2142); MEN1, In, C_7, 16, driver, essential, PPPEASWDCRWHSPRP (SEQ ID NO: 2143); TOP3A, Del, C_7, 16, essential, PPGLASPLAACRLTSP (SEQ ID NO: 2144); SMG5, In, C_7, 16, essential, PPHRGEQRARSKSPPH (SEQ ID NO: 2145); BRF1, In, C_7, 16, essential, PPLVHSWAEEAADEAA (SEQ ID NO: 2146); BRPF1, In, C_7, 16, essential, PPPAPHHEFPASAQAG (SEQ ID NO: 2147); DHX37, Del, C_7, 16, essential, PPSRPPCSWNWRTRTR (SEQ ID NO: 2148); FLT4, In, C_7, 16, driver, PPDLEHHGGVTRHRHR (SEQ ID NO: 2150); TERT, Del, C_7, 16, driver, PPRPSPPACAATCPTR (SEQ ID NO: 2151); CUX1, In, C_7, 17, driver, high expression, PPSFSVAPQPGGAGFQY (SEQ ID NO: 2152); HCFC1R1, In, C_7, 17, high expression, PPHDLLPSPAPTQEPLL (SEQ ID NO: 2153); FNDC3B, In, C_7, 17, high expression, PPHHQSTSSNTVRRKFM (SEQ ID NO: 2154); PDS5B, In, C_7, 17, high expression, PPPSSQICHSLYPCDIF (SEQ ID NO: 2155); CHD3, Del, C_7, 17, high expression, PPVLFKADQSESSLSSG (SEQ ID NO: 2157); UBR4, Del, C_7, 17, essential, PPCWTSHLMQMTRPWLN (SEQ ID NO: 2158); THOC1, In, C_7, 17, essential, PPRWRKIFKDGRAYIKH (SEQ ID NO: 2159); IGFBP5, Del, C_7, 18, high expression, PPAPWAASWSRSRAAAAA (SEQ ID NO: 2160); SEC24C, In, C_7, 18, high expression, PPPVFSAPGSYRQTCGCL (SEQ ID NO: 2161); TACC3, In, C_7, 18, essential, PPHASFSGLLPPRLGQNG (SEQ ID NO: 2162); CLASRP, In, C_7, 18, essential, PPSAHHHLPRTGVGRTEV (SEQ ID NO: 2163); PTCH1, In, C_7, 18, driver, PPQRRLVATPLQTAQRRF (SEQ ID NO: 2164); RYR1, Del, C_7, 19, high expression, PHPAQLSPLTTSTPCWGIS (SEQ ID NO: 2165); ABR, Del, C_7, 19, high expression, PPFSPQNSSGTHCTSPPTC (SEQ ID NO: 2166); LRP1, In, C_7, 19, high expression, PPQPVLLCQWPLHPHLLDV (SEQ ID NO: 2167); MARCKSL1, Del, C_7, 19, high expression, PPRRPPRRRRNSLSRSLSN (SEQ ID NO: 2168); TAF4, Del, C_7, 19, essential, PPHRAAPLSPQGPRRPPRS (SEQ ID NO: 2169); ZMIZ1, Del, C_7, 19, essential, PPTCRAIVTMTSCLYLRTT (SEQ ID NO: 2170); GATC, Del, C_7, 19, essential, PQVISLCQSWMNKSHSHTA (SEQ ID NO: 2171); TCF20, Del, C_7, 20, high expression, PPCRTRPRKAASAQSSRSGG (SEQ ID NO: 2173); ABR, In, C_7, 20, high expression, PPHFLRRTQAEHTVLLHRRV (SEQ ID NO: 2174); UBQLN2, In, C_7, 20, high expression, PPWLHRLWWPHGAYCVQRCT (SEQ ID NO: 2176); CREBBP, In, C_7, 20, driver, essential, PPTPGPAPSCSGGSGSADRA (SEQ ID NO: 2177); DNMT1, In, C_7, 20, essential, PPQTPFQTSHAQEEQVRWRG (SEQ ID NO: 2178); DHX37, In, C_7, 20, essential, PPRAAPRAAGTGGQGHVEGS (SEQ ID NO: 2179); HCFC1, Del, C_7, 20, essential, PPVRPTRRAPLTRPPLSLPT (SEQ ID NO: 2180); PAX5, In, C_7, 20, driver, PPRWTGQLLSTDADRDGAWE (SEQ ID NO: 2181); BCORL1, Del, C_7, 20, driver, PQAPLRLWSWCGTSQTYFGS (SEQ ID NO: 2182); BRD8, In, C_7, 21, essential, high expression, PPPEVTHCDGSLSYRFCLPRR (SEQ ID NO: 2183); TMX2, Del, C_7, 21, essential, high expression, PPYIWALSISSTSMIKPLMRN (SEQ ID NO: 2184); TCF20, In, C_7, 21, high expression, PPLAEQDRERQPQHRAVGAGV (SEQ ID NO: 2185); UBB, In, C_7, 21, high expression, PPRPAEAHLCRQAAGRWPHSF (SEQ ID NO: 2186); ATP2A1, Del, C_7, 21, high expression, PRSERKWSWMTLPGSWSMRRT (SEQ ID NO: 2187); POLR3E, Del, C_7, 21, essential, PPRLLLPRMSRRCLPSGSLET (SEQ ID NO: 2188); GAB2, In, C_7, 22, essential, high expression, PPQAKSGRNTSMGQSSAETANQ (SEQ ID NO: 2189); AFF3, Del, C_7, 22, driver, PPTSPAPRLNRHPLTSGSWING (SEQ ID NO: 2191); JAK1, In, C_7, 23, driver, high expression, PPVDRPQHTEWLSWSNLYRIRHQ (SEQ ID NO: 2192); SSH2, In, C_7, 23, high expression, PPRTFICPQGTRNEQRQREIQWV (SEQ ID NO: 2193); CHERP, In, C_7, 23, essential, PPPWLQRAAPTHAATGPTPHQPR (SEQ ID NO: 2194); GTF3C1, In, C_7, 23, essential, PPRGAGTSFSQPPGHQEARQLGQ (SEQ ID NO: 2195); CSF3R, Del, C_7, 23, driver, PPARAIATRPGGWNRMGEPRGFC (SEQ ID NO: 2196); SHC1, In, C_7, 24, high expression, PPGGAAFPISFIPGAHPASSAWGR (SEQ ID NO: 2197); MRPL38, In, C_7, 24, essential, PPLPCPRLRHPPSCLPALQAGPAD (SEQ ID NO: 2198); TP53RK, In, C_7, 24, essential, PPPGTAEHCAHRLWAEFHFSTSRG (SEQ ID NO: 2199); IL21R, Del, C_7, 24, driver, PPGATSASGWSFLRHFRALDPRPA (SEQ ID NO: 2200); SUFU, In, C_7, 24, driver, PPGLRFALSPGTARHLRRVPPPLP (SEQ ID NO: 2201); MLLT1, In, C_7, 24, driver, PPPEATPAQQQGVRPEEPRVLQQA (SEQ ID NO: 2202); RARA, Del, C_7, 24, driver, PRQAAVAPASAPAPTEAARPPTPR (SEQ ID NO: 2203); NACA, Del, C_7, 25, driver, high expression, PQIFPFLWALILHLYIRVLLVLSNF (SEQ ID NO: 2204); TULP4, Del, C_7, 25, high expression, PPASAPTSPLPSSPPSSPQFQIRTT (SEQ ID NO: 2205); MRPL12, Del, C_7, 25, essential, PPRYSSWSRTSPASLSWKSQTSTSS (SEQ ID NO: 2206); ETV1, In, C_7, 25, driver, PPHTIQHASVPTASCISKLNSYTET (SEQ ID NO: 2207); IL21R, In, C_7, 25, driver, PPPELPPPVGGHSSATFEPWTPGQL (SEQ ID NO: 2208); CBFA2T3, Del, C_7, 25, driver, PPPSRHTHIERTGLRRCPTAVFMAA (SEQ ID NO: 2209); CRTC1, In, C_7, 25, driver, PPREPWPAIDGDRHRLGAGSAAVPH (SEQ ID NO: 2210); TSC22D1, Del, C_7, 26, high expression, PLPPPSHRAPHSPPPSQHRRAQDQPH (SEQ ID NO: 2211); NDUFA10, In, C_7, 26, high expression, PPPPGDLHRCARSRGPEADSEERRST (SEQ ID NO: 2213); HERC1, In, C_7, 26, high expression, PPPVWGAERSQHSHGAGQAPAALPLL (SEQ ID NO: 2214); MYLPF, Del, C_7, 26, high expression, PPTWAATSTTKTSATSSRTATPRTRS (SEQ ID NO: 2215); TNPO3, Del, C_7, 26, essential, PPIPYQMWLKCSGRSCRLTDRLFVDG (SEQ ID NO: 2216); ZFHX3, In, C_7, 26, driver, PPRGSFPRQRPCQRIPQTRRTEKHPP (SEQ ID NO: 2217); CUX1, Del, C_7, 27, driver, high expression, PLLLSCPATRGSRLPILMVHTSSHQRG (SEQ ID NO: 2218); TSC22D1, In, C_7, 27, high expression, PPCHHPATGHHTAPRPASIAGLRTNRI (SEQ ID NO: 2219); EPHX1, In, C_7, 27, high expression, PPPAARRPYPEALADGARLARLFLRVL (SEQ ID NO: 2220); CHD3, In, C_7, 27, high expression, PPRLPPLPAAAAAAATAAPAAAAPAAR (SEQ ID NO: 2221); MYLPF, In, C_7, 27, high expression, PPRRGRQRRLQKHLLRHHARRRQGPGV (SEQ ID NO: 2222); CYC1, In, C_7, 27, essential, PPQLSVVSPWPPLFLGPHQHPEGFPGI (SEQ ID NO: 2223); PABPN1, In, C_7, 28, essential, high expression, PPGSSGPWAWFGSPRQPRGGGGAGTGRG (SEQ ID NO: 2224); COL4A3BP, In, C_7, 28, high expression, PPHTGAGTLRFAIPRPFTPRTGRLLRRS (SEQ ID NO: 2225); BRD4, Del, C_7, 28, driver, essential, PPSHNPHPLQLPSPYRATHPSSRPPHSL (SEQ ID NO: 2226); MED15, In, C_7, 28, essential, PPPAVPAARPAQLTAQLQRQLWPCPISQ (SEQ ID NO: 2227); WBP11, In, C_7, 29, essential, high expression, PPWAVPTSSLAKPWGFKCPTQLDSATQGG (SEQ ID NO: 2228); MBD2, In, C_7, 29, high expression, PPRMEEGGSDPKIWAKCWQERCLLLQSKW (SEQ ID NO: 2229); GATA3, Del, C_7, 29, driver, PPAACWAAPPPASDASPGPRPGPAQAGSV (SEQ ID NO: 2230); EP300, Del, C_7, 30, driver, essential, high expression, PPTPVLPQGCSLSLLHTTFPHRQVPHILDW (SEQ ID NO: 2231); PHACTR2, Del, C_7, 30, high expression, PLSLLRISALLPQTLQLSSSALELTCPSLP (SEQ ID NO: 2232); PIK3CB, In, C_7, 30, high expression, PPPGGPRASGFQLSRPVRSRICCRLPATDE (SEQ ID NO: 2233); ATXN2L, In, C_7, 30, high expression, PPPRRGAPEWGACTLSFHTLTLPLHRTPPR (SEQ ID NO: 2234); MPRIP, Del, C_7, 30, high expression, PQLLSQTPRLPPCLHTEEPSHWTGGPRSPP (SEQ ID NO: 2235); CREBBP, Del, C_7, 30, driver, essential, PHPRPSPLLQRWKRLGRSSVRPSSSSTCTG (SEQ ID NO: 2236); HGS, Del, C_7, 30, essential, PPSSSPPWPSNRRHRGRRHRAARPSSFHST (SEQ ID NO: 2237); GATA3, Del, C_7, 30, driver, PPAACWAAPPPASDASPGPRPGPAQKAGSV (SEQ ID NO: 2238); TCF7L2, In, C_7, 30, driver, PPLRLHCQHSSLQGHEKEPLLTKSWGALVY (SEQ ID NO: 2239); FGFR3, Del, C_7, 30, driver, PPRKAWAPPPCTRSPASRSSDRCPWSPTRP (SEQ ID NO: 2240); MKL1, Del, C_7, 30, driver, PQYAASPLPIAAPARAPLGPVGWHVRTAPH (SEQ ID NO: 2241); APOA1, In, C_7, 31, high expression, PPPEPLGSSEGPGHCVRGCAQRQRQRLCVPV (SEQ ID NO: 2243); ITM2B, Del, C_7, 31, high expression, PPTPSRWTARTQMMWYQLAKEEPGVGACALD (SEQ ID NO: 2245); HGS, In, C_7, 31, essential, PPPAAAPRGPATAGTGAAGTGQRGPAHFIRL (SEQ ID NO: 2246); ETV4, Del, C_7, 31, driver, PPDKSPSSPLPLVPLDSRPYSPFPGQSNGIS (SEQ ID NO: 2247); DNM2, In, C_7, 32, driver, essential, high expression, PPPDSCSRGGSSLLLGAPNPIPAWTPERVCQQ (SEQ ID NO: 2248); DCTN2, Del, C_7, 32, essential, high expression, PQIAALSLMNYILGLSRTSSLKLPKSQNLKSA (SEQ ID NO: 2249); DNMT1, Del, C_7, 32, essential, PPNPFPNLARPGGASPMERLSVQETLLPQPPK (SEQ ID NO: 2250); INTS1, In, C_7, 32, essential, PPPEASPSHPGSSEKPQPVPPPRAPLVPQPKP (SEQ ID NO: 2251); CIITA, Del, C_7, 32, driver, PPGPWQSWPSWPGSWAADIKVPYRRTSSHPQT (SEQ ID NO: 2252); SSH2, Del, C_7, 33, high expression, PPCPLILSHQAPNPVARLKSQISVQIALTFLVP (SEQ ID NO: 2254); ST6GALNAC6, In, C_7, 33, high expression, PPQLLQPAAPPPAHALPLLRAQGAGRMCHLHPE (SEQ ID NO: 2255); TACC2, Del, C_7, 33, high expression, PPRGRTWQQTWGSRHSSWTKISRESHPAQGKAG (SEQ ID NO: 2256); ANKRD17, Del, C_7, 33, high expression, PPRRRRSAAALAAAAELARPRLLVGWCESATCS (SEQ ID NO: 2257); SEC24C, Del, C_7, 33, high expression, PPSIFSTWIIPANVWMLMTALSYPWALMNSWPL (SEQ ID NO: 2258); PDS5B, Del, C_7, 33, high expression, PPVKPNMPFIVSMRYFLVKRPSLHRYLSLCIRA (SEQ ID NO: 2259); PELP1, In, C_7, 34, essential, high expression, PPPATPTTLRCHTTPYSPHWATNSLPSCASEGGA (SEQ ID NO: 2260); TNFAIP3, Del, C_7, 34, driver, high expression, PPSSVAGPPPVIILAMPSATATATNAFSSSRCMA (SEQ ID NO: 2261); MARCKSL1, In, C_7, 34, high expression, PPQGDPQEEEEILFQEAFQIERPVLQEKSEGGWG (SEQ ID NO: 2263); C3, In, C_7, 34, high expression, PPQVLVVRSICHRAAKDRPAGSGSQGCCLPSFHQ (SEQ ID NO: 2264); PABPC4, Del, C_7, 34, high expression, PPRNRSRCWENACSHSSKQCIQIWLGRSRECCWR (SEQ ID NO: 2265); PATL1, Del, C_7, 34, high expression, PPRVQKMIGTFLNEHYQGGQLHLSLAVLLLELSP (SEQ ID NO: 2266); GTF3C1, Del, C_7, 34, essential, PPRGRHLLLTAPGAPRGAPAGPVRMGRPTPRAPR (SEQ ID NO: 2268); YLPM1, Del, C_7, 35, essential, high expression, PLEIRQLSSKSLWRVGPKTRVLNSSKPPLSQIPLR (SEQ ID NO: 2269); RDX, Del, C_7, 35, high expression, PLHLHHHQSFLQQKTNMMNTMRIMLKLVLNYQMKG (SEQ ID NO: 2270); SHC1, Del, C_7, 35, high expression, PRRSCLPHQLHPWGPSCLLCLGTIVPLPCAPSSPG (SEQ ID NO: 2271); ZMIZ1, In, C_7, 35, essential, PPPALPPPGHAQQHGRPRETPQPPHAGNYATRWQL (SEQ ID NO: 2272); MRPL24, In, C_7, 35, essential, PPPLPLWDEPPRLCCRQEEEPPMDQAAPSGCGTHL (SEQ ID NO: 2273); SLC16A1, In, C_7, 35, essential, PPRWRLGLGSGNWSFHFHRLLLCISQINYCLLQRD (SEQ ID NO: 2275); CIC, In, C_7, 35, driver, PPKPGLHCGHQHNPTCSHHSAQGPASPCHCHPSPD (SEQ ID NO: 2276); TF, Del, C_7, 36, high expression, PPGWMPRCTWAMSMSLPSGIYGKAHAQKPQQMNASL (SEQ ID NO: 2277); ABR, In, C_7, 36, high expression, PPQHRRPALGPRAQQAAPKQAGPTNRGDQELAHGRD (SEQ ID NO: 2279); UBB, Del, C_7, 36, high expression, PPTSRGSSLQASSWKMAALFLTTTSRKSRPCTWSCA (SEQ ID NO: 2280); VAMP2, Del, C_7, 37, high expression, PLLPRLGRVVPLHPLQTSPVTGDCSRPRPRWMRWWTS (SEQ ID NO: 2281); CTSA, Del, C_7, 37, high expression, PPAPTQQLLPPTSTTRTCGRPSTSRSSCHNGTCATFW (SEQ ID NO: 2282); TRIB1, Del, C_7, 37, high expression, PPARPAARSPRLPLRGPAEAPGARRGPAASPTTCCCP (SEQ ID NO: 2283); ATXN2L, Del, C_7, 37, high expression, PPKARCPRVGCLHSQLPHPHPTPTSDTPKLPFHPPGN (SEQ ID NO: 2284); NCAPD2, Del, C_7, 37, essential, PPSGCLRNTGCLSDCGRQSQKALSTQTHSGSHSKRWQ (SEQ ID NO: 2286); TARBP2, Del, C_7, 37, essential, PPWNCSPLSPLSSLSATPLVLCRSWWCRKAGGCRSTQ (SEQ ID NO: 2287); KLF3, In, C_7, 38, high expression, PPASIVARESPFGHRAAWEETFTCGIPGYSKEAEDTQM (SEQ ID NO: 2288); SETD5, In, C_7, 38, high expression, PPLSRFFRIIPLFHVLFQPRPPCVHRLVGPIYGDTRVF (SEQ ID NO: 2289); PABPC4, In, C_7, 38, high expression, PPPGTEADAGRTLVPTHPNNAFKSGWEDHGNAAGDRQL (SEQ ID NO: 2290); OPTN, In, C_7, 38, high expression, PPPPGPPKPGHVYPGGAAAADERAPDREPPAERSHEAK (SEQ ID NO: 2291); ITSN1, Del, C_7, 38, high expression, PPWLTGLPLLYNLCLHLLILQPHCQRVLPLVDLVQGHN (SEQ ID NO: 2293); SEC16A, In, C_7, 38, essential, PPRVGSCRLGSHAATSVSLTRSRNKETRTGSQERNEGT (SEQ ID NO: 2294); BAG6, Del, C_7, 38, essential, PPSHFLRSQWKFREQKELPLSLRNGSLLSSRTFRASGR (SEQ ID NO: 2295); RCC1, In, C_7, 38, essential, PPSRCHPQKQEGEGHEGRCLPPRSWRPLLPRCLRAEPS (SEQ ID NO: 2296); CAD, In, C_7, 38, essential, PPTRYHCQNPGADQSHCACCGPGATGHRTLQSAAHCQG (SEQ ID NO: 2297); PCNT, Del, C_7, 39, essential, high expression, PQPGMYPLATPGTLPEAADWQQQPPHTVGEEPLHPQIQD (SEQ ID NO: 2298); CHD3, Del, C_7, 39, high expression, PPAATSSRRRRRRRHRCPRRRRPRRQIRMTFGCCRQHWV (SEQ ID NO: 2299); TNS1, In, C_7, 39, high expression, PPGSGQDTPVCSGPETSQPSGHPVAPHRSYQKTDPARGR (SEQ ID NO: 2300); CHERP, Del, C_7, 39, essential, PPMASTGSPHTCGDRAHPTSTTMTPAWSPMCPTSISLLG (SEQ ID NO: 2301); CARD11, In, C_7, 39, driver, PPPEPQQHHVNHRRAPGKRLHRQTLQGGRAPSQHSRRRQ (SEQ ID NO: 2302); ILF3, In, C_7, 40, essential, high expression, PPPGYECPDAVEPAEARAAVQAGVPDWARPCPHLYHVCGG (SEQ ID NO: 2303); NES, In, C_7, 40, high expression, PPHTSGTLSCCRCRDQSPGCSSLSAPDTGWEETGSRAPAG (SEQ ID NO: 2304); BRD4, Del, C_7, 40, driver, essential, PPKCCWRMKSHLPHPSPPCRCSCTCSSCRRCSPLRRYSLP (SEQ ID NO: 2305); CAD, Del, C_7, 40, essential, PHKISLPKPWSGSKPLCMLWARSYRSQDPSICSSLPRMTS (SEQ ID NO: 2306); RCC1, In, C_7, 40, essential, PPSRCHPQKQEGEGLTQVPQHRTRLGADTRPGRRGPAGAG (SEQ ID NO: 2307); ATP2B4, In, C_7, 41, high expression, PPQKAQDFLRISVGSSSRCHAYHPGDCSHHLPGPVLLSPCW (SEQ ID NO: 2308); CCDC92, Del, C_7, 41, high expression, PPSPPTEAASSTARPAKSRTRPTSGWHIGSTTPPRRRPSPR (SEQ ID NO: 2309); FOXN3, Del, C_7, 42, high expression, PHPLPTLTCPTMPGRTPTANPPTPSAASYLWPSRTLQPSACQ (SEQ ID NO: 2310); LRP1, Del, C_7, 42, high expression, PPTSSPVPVAAASPSPGRVIWMTTVGTALMSLLRVPIPPASP (SEQ ID NO: 2311); MED15, Del, C_7, 42, essential, PPSRPRSPASPAHSPTPTSALALPHLPVASCPAPHRSPPRAQ (SEQ ID NO: 2312); PFAS, Del, C_7, 42, essential, PRHPCQPLWTWSSNGCWARCLGRSSSCRGSPPCCSLWPCPQG (SEQ ID NO: 2313); WDRS, In, C_7, 43, essential, PPRVFCEVLPERQIHPGRHAGQHSEALGLQQGEVPEDVHWPQE (SEQ ID NO: 2314); SNAPC2, In, C_7, 43, essential, PPSHREGRAQRTEIALASSWDLSPEPVPGAPGASGSGSHPCQV (SEQ ID NO: 2315); BCL9, In, C_7, 43, driver, PPCFGVCCCFTCPPQVSITSCPVTWMDLFSKTSPSESWDPSKP (SEQ ID NO: 2316); ZFHX3, In, C_7, 43, driver, PPPAAAAAAATTGAAASPAASSPAATHTTAPTATAAATQGQRQ (SEQ ID NO: 2317); SF1, Del, C_7, 44, essential, high expression, PPGCPRSPGPKEASSRALRLHPGLGRGCWLPGRRRPRPWARWGP (SEQ ID NO: 2318); EIF4G3, Del, C_7, 44, high expression, PPNNIQFNHRGQVLFILDQDLGTSPMLMERLFTQVSRCISQHLS (SEQ ID NO: 2319); CTSD, Del, C_7, 44, high expression, PPSASQSSSTRAPPTCGSPPSTANCWTSLAGSTTSTTATSPAPT (SEQ ID NO: 2320); MPRIP, In, C_7, 44, high expression, PPSSSPRRLGFPPVSTPKSQVTGQEVHGALRDARPAEFQERLAD (SEQ ID NO: 2321); MEN1, Del, C_7, 44, driver, essential, PPGSLLGLSLAQPEALKVAARLRCQHPQHHHRRRVQCSLSRVRR (SEQ ID NO: 2322); ATXN2L, Del, C_7, 45, high expression, PPKARCPRVGCLHSQLPHPHPTPTSDTPKFNLIPPSSSPSTPRGT (SEQ ID NO: 2325); REV3L, Del, C_7, 45, essential, PQVGKKLWQLCWIMTCLRLFTRNHFAVILLMYQKSPGRLVDGSSW (SEQ ID NO: 2327); ZFHX3, Del, C_7, 45, driver, PPSSSSSSSNHRCSSLPRRQQPSRHPHHSSHCNSSSNARTKTVRK (SEQ ID NO: 2328); CIC, Del, C_7, 45, driver, PQAWSTLWPPAQPHLQPPFCPRARQPLPLPPQPRLALSPAPQQVP (SEQ ID NO: 2329); TACC2, In, C_7, 46, high expression, PPPGGELGSRPGAHGTHPGPRSAGNPILPRGRLDKRSCIRVAPTIF (SEQ ID NO: 2330); ATXN2L, In, C_7, 46, high expression, PPPRRGAPEWGACTLSFHTLTLPLHRTPPSSISSLPAAPLPPPGEL (SEQ ID NO: 2331); ATXN2L, In, C_7, 46, high expression, PPPRRGAPEWGACTLSFHTLTLPLHRTPPSSPSTPRGTEDCPGRDL (SEQ ID NO: 2332); CARD11, Del, C_7, 46, driver, PPGAAAASCQSPPSPRETTPSSDATRRTRPIAAQSKKTMTAAGLTP (SEQ ID NO: 2333); PCBP2, In, C_7, 47, essential, high expression, PPEGRDHPVPAQAVQLSGHLCRWSGQVQHRQRQCELSPHHPVHVPQP (SEQ ID NO: 2334); EIF4H, Del, C_7, 47, high expression, PPWAAASEMALPSVDPTWISENPQKRKEHRDHDSSLNLEQSRRPSIK (SEQ ID NO: 2335); BRD4, In, C_7, 47, driver, essential, PPPATTPTRSSSPARTEPPTHHRGHPTACEDKEGSEEESRHHHPHHH (SEQ ID NO: 2336); MLLT1, Del, C_7, 47, driver, PPRSHPRPTARCQAGGAPSPAASLRRSSRRAPTTRPTRMSWWSYTGG (SEQ ID NO: 2337); ANKRD17, In, C_7, 48, high expression, PPRGGGGRRRRWRQQQSSLGLVSSWDGASLRPAPEEEAAAAAAPQGQA (SEQ ID NO: 2339); PAWR, Del, C_7, 48, high expression, PREGAAATPLGSPPRGLWAPRRPPLPTSSTTTSRAARRPHLPSPVPGA (SEQ ID NO: 2340); BRD4, Del, C_7, 48, driver, essential, PPIRPQASSHPRRSLPSLSKSSSTTIHPGTTSRTPTQPVTSAKPPPRL (SEQ ID NO: 2341); ATP2B3, In, C_7, 48, driver, PPAPVPQPEQQRHRQRHLPDHACHQVSYLFSVFLQSREPAPQRGDVPL (SEQ ID NO: 2342); SORBS2, Del, C_7, 50, high expression, PPLGAGEAASLTPWCPFTLKSPAMSSPEHTWSFPTATRTGLCPTTVTTFT (SEQ ID NO: 2343); PPRC1, In, C_7, 50, essential, PPKKDACPSHSRGGLPMECQAPSGHHHQTCLVLGPSCPSAPMHSCLPGAA (SEQ ID NO: 2344); TRIO, In, C_7, 50, essential, PPQQPPAEGGLLLELHPRLPRQPTRLLHLPGGQRLPPAADTPPRGPWQGY (SEQ ID NO: 2346); ETV4, In, C_7, 50, driver, PPQTNRHQVPCPWCPWTVAPTALSPGRATEFPEILWHLPAPPWPWVPRGT (SEQ ID NO: 2347); ATXN2L, Del, C_7, 51, high expression, PPKARCPRVGCLHSQLPHPHPTPTSDTPKVSSLARRLDFQEEPMTGFVSSH (SEQ ID NO: 2348); CTSD, In, C_7, 51, high expression, PPPVLHSRLRHGLLQPVGPLHPLQTAGHRLLDPPQVQQRQVQHLREEWYLV (SEQ ID NO: 2349); USP9X, Del, C_7, 52, essential, high expression, PPSNRIRLHRLILPMKIPRQLPQMSKVKVMPHHSLKMRNLHFHILTWPSWMT (SEQ ID NO: 2350); KLF3, Del, C_7, 52, high expression, PRKHCCKRITLRSSCSLGRDLYLWNPRILKGSGGYTDVIMMDATKCTLKAPT (SEQ ID NO: 2351); TRIO, Del, C_7, 52, essential, PPAAPCRRGAPSGAPSPPPPPADPAPSPSRGTATPSSGRHPATRPLARILTA (SEQ ID NO: 2352); DLGS, In, C_7, 52, essential, PPSQAEQLQDCGRCQQEDPGATRCLHQKVPAGAWGALVWWEPAWGVCGRGGG (SEQ ID NO: 2353); BCL3, In, C_7, 52, driver, PPPRPGPAGGALLPRSLTAFVPHSGHGLPVSSGEPAYTPIPHDVPHGTPPFC (SEQ ID NO: 2354); MDM2, Del, C_7, 53, driver, essential, high expression, PPFHHIATDVGPFVRIGFLKIKGKIKGKSLRKPNWKTQHKLKRALMFLIVKKL (SEQ ID NO: 2355); IGFBP4, Del, C_7, 53, high expression, PPWAARSWCESRAAAVAPLAPWAWGCPAGCTPPVAARACAATRPEGWRSPCTH (SEQ ID NO: 2356); BAG6, Del, C_7, 53, essential, PLVLGGLGPLFMTGMPTAMSWLEPSIFLVTALLWMFTSTWNRPRFRVSPGYGW (SEQ ID NO: 2357); FANCE, Del, C_7, 54, driver, PQLSYSFFTNVVPARWTCCVPSCSSLSSQTSVSCGSAPGCWPFHLISASAMLLC (SEQ ID NO: 2358); ATXN2L, Del, C_7, 55, high expression, PPKARCPRVGCLHSQLPHPHPTPTSDTPKVSSLARRLDFQEEPMTGFLPFHPPGN (SEQ ID NO: 2359); WDR77, In, C_7, 55, essential, PPPSAPGGPGVESSPKCARLHGTAVGGCAVPVRWGASPRGLQPEWALLGRLPLAF (SEQ ID NO: 2360); PABPN1, Del, C_7, 56, essential, high expression, PRELRALGLVREPPAAKRRRRSRDWSRVTRGTAPLRTRSWKLSKLESGRWRKKLRS (SEQ ID NO: 2363); PRKAG2, Del, C_7, 56, high expression, PRTQGRGSARLPSRARPGLHWHHRHTMLPPKPRRWRRPWDPRKPACWRSWSSRTKQ (SEQ ID NO: 2364); GRINA, Del, C_7, 57, high expression, PPTPMDSHRSSQDKTLTHPSMETTRRRVPHPTMTTRTSLPPTGMTRASDRPSSARCS (SEQ ID NO: 2366); BCL9, Del, C_7, 57, driver, PLFWGLLLLHLSTSSLHHFLPRHLDGPLLQNLPFRVLGSLQTIKHPSPWPPQPCWEM (SEQ ID NO: 2367); RXRA, Del, C_7, 58, high expression, PPWASMASSRSPPTPQETWLPSPSTSAPSAGTAPQASTMECTAARGARASSSGRCART (SEQ ID NO: 2368); NAB2, Del, C_7, 58, driver, PLQGEESLRGLGLGGWQQVGLGVVQTDWSQRWYAWWWKVWRGSSGASQGGMLGRSHPC (SEQ ID NO: 2369); NUP214, In, C_7, 61, driver, essential, PPGVREAHGAPCDILCNHHLSSTTSSHQHFLNCRFWQSASHQCRILWGHQFWWDISKCWQD (SEQ ID NO: 2370); LETM1, In, C_7, 62, essential, high expression, PPRRGGGEEVPGAAGAGRAEALLPWLPPAMDRHQDRGTHALAHPQRPQPDPPGAQAVSPDLR (SEQ ID NO: 2371); BCL11A, Del, C_7, 62, driver, PPSLLCNPPLLPPSPRSSPSHASSAARRSNFRATWWCTGAATRARSPTSATCATTRAPRPAS (SEQ ID NO: 2372); ATXN2L, Del, C_7, 63, high expression, PPKARCPRVGCLHSQLPHPHPTPTSDTPKVSSLARRLDFQEEPMTGFFNLIPPSSSPSTPRGT (SEQ ID NO: 2373); TCF20, Del, C_7, 63, high expression, PPRGAALAISMGVRVMWASFKHSTLALAVCHIISRITLGLSLQGVLSTNSRLPASSSSSKSSS (SEQ ID NO: 2374); TRIB1, In, C_7, 63, high expression, PPRLALQPAAPACRSGGRRRLRERAGAQPHRRLPAAAPSRARACVPGAVHPHWTRAALQGVSH (SEQ ID NO: 2375); TRIO, Del, C_7, 63, essential, PPCWSPLQPRARQRQTRCQVRPPPGPPCLPLARPPRPAPARPAGGPPARTPRGPSEKRSRSPR (SEQ ID NO: 2376); PCBP2, In, C_7, 64, essential, high expression, PPEGRDHPVPAQAVQLSGHLCRWSGLYHSRTVCHSTARFDQAAPVGNATVSFSHDAWQHRIQWH (SEQ ID NO: 2377); PTCH1, Del, C_7, 65, driver, PPEKACGHPPTDRAETLLKFLLKGILALAIGPAGALAGPVLTTLGTQRPLPWAAPCPATASPSPL (SEQ ID NO: 2378); TACC3, Del, C_7, 66, essential, PPCQLLGALTTSTGTKWMTQTSSRSEVTPSLVAVRPSPQKALRPGWASQRLNSCMLGLPRRSQVPV (SEQ ID NO: 2379); PSMC4, Del, C_7, 66, essential, PPEASSCMAHLAVGRPCWQRRWHITQQLHSSGSWARSLYRSIWVRAPAWSGMCSAWPRRMHLPS SS (SEQ ID NO: 2381); CCDC92, In, C_7, 67, high expression, PPHRLRPKRRAAQPGPRKAAQGPRRGGTSDPPRHPAAGPARGEDPGGRPGERRQGGEEALRDGQN CV (SEQ ID NO: 2382); CDK9, Del, C_7, 67, essential, PPLTCGVLGASWQRCGPAAPSCRATRSSTNSPSSVSSAAPSPLRCGQTWTTMSCTKSWSWSRARSG R (SEQ ID NO: 2383); MUC1, In, C_7, 67, driver, PPSPWCHLGPGQQARLGLHRPSSPQCHLGLRLCIRLSFYSGAQRHLCQGYHNPSQQEHSILNSQPPL (SEQ ID NO: 2385); MIF, Del, C_7, 68, high expression, PPSTSRCTWSRTSSWPSAAPASRARSAACTASARSAARRTAPTASCCAACWPSACASARTGSTSTIT T (SEQ ID NO: 2386); ST6GALNAC6, Del, C_7, 68, high expression, PPTTAASGPASSACPTTTTSPRGRTNVSPTSRMSTVARATTTASSPRKGSSHRGPSCMASPSPTPPGP (SEQ ID NO: 2387); OSBPL2, Del, C_7, 69, high expression, PPSVRSTRKVSTMTSCSMAPSTPSSSSGAKAWRRSPEAPSPWSCSNIMKPTPGPTPPAASTTSSSGSC G (SEQ ID NO: 2389); PTPN23, In, C_7, 69, essential, PPPTHPALPRSRSRPSASPLRGSAFPQPWAPSASPSPTGIWSCPFYQTHGPPGSPSYHSRALVCWPVH P (SEQ ID NO: 2390); GAB2, Del, C_7, 71, essential, high expression, PPSQVRQKHLDGAVLSRDRQSVKIADLSLPPSPDATPSLQWTTADFTELLPVRPTSTHSVVERVQAG LLNP (SEQ ID NO: 2391); UBQLN2, Del, C_7, 71, high expression, PLAPPALVAPRGLLCPALHLVKPRVLHQNLDPTSSSFSKWCRPWLEQMLHSCRIQKSDFSNNWNSS TQWGS (SEQ ID NO: 2392); EIF4G3, In, C_7, 71, high expression, PPPTISSSTTGARSFLSWTRTWGLPQCLWNAFLPKSAGVSVSTYHSAYAATAASSQEREKNYKNSG SKPGR (SEQ ID NO: 2394); NFATC2, Del, C_7, 71, driver, PPRCGRPALTPRRCLPPHPRPACLATSTRPWSSWGPASRARGETRLQNPSCWFRPLGPSRWCLPFSP AASQ (SEQ ID NO: 2395); FLT4, Del, C_7, 71, driver, PPRYMRRRPPPPASTRVTAARPSPARPTGCPCLSASSGTGGPGHPARCLPSVVSGGGSSKTSCHSAV TGGR (SEQ ID NO: 2396); SUFU, Del, C_7, 72, driver, PRPSLRSFPRDCTPSTESAAAFTLTSRTRSRLPLSSSTGWVAQTPWTMLACTGMWGALLLTSPSTGT TSASA (SEQ ID NO: 2399); ANKRD11, Del, C_7, 73, essential, high expression, PPPRP SVPLTAPPRTLWHKLRPQTVPAPRTTLRPPVPPPQPKALLAASSRKPQNQNPRPKPRRPPEWR RSLSA (SEQ ID NO: 2400); BRD4, In, C_7, 73, driver, essential, PPPSAPRPAATPAAACQASASHPAPPFTPAPQVGPLLNRSPPRSPLPAYDTFPPDVTVPEPDPPVSTPA KRPA (SEQ ID NO: 2401); PCBP2, In, C_7, 74, essential, high expression, PPEGRDHPVPAQAVQLSGHLCRWSGLYHSRTVCHSTARFDQAAPVGNATVSFSHDAWQHRIQCRF GCICSDYFS (SEQ ID NO: 2402); SEC16A, Del, C_7, 76, essential, PQGGIVPTRVPRSHLCLSHPLPKQRDPDRQPRKKRRNLRRVNPGSFVGYLERKRQKLICQMTRTNR LFGMKRKTSG (SEQ ID NO: 2404); IGFBP4, In, C_7, 77, high expression, PPRGLRGAGARAGLRLLRHLRPGLGDALRGVHPPLRLGPALLPAPRGGEAPAHTDARARRVHGAG GDRGHPGKPAAL (SEQ ID NO: 2405); NCOR2, In, C_7, 77, driver, PPPGGRS SHQAS SP SPTATAKPAAGERRP SAAWQQPPGQEQEPGTPRRQGGLRSRGPEAAWGPPLL DFRPALPRAPP (SEQ ID NO: 2406); IGFBP5, In, C_7, 79, high expression, PPQPPGLRAGQGAGLRLLHDLRPGRGAVVRRLHRALRPGAALPPPAGRGEAAARPAARPRGLPQR KELPRASQDRERLP (SEQ ID NO: 2408); FAM193A, Del, C_7, 80, high expression, PPSLMTSTFTSSHFKWILLLTILLRGARPVCHLQARGPAPALPSQFSSTPGSSSQTVARHQLLMMKM LHHCQPNLLIHH (SEQ ID NO: 2410); TERT, In, C_7, 80, driver, PPRGLHHQRAQLPAQHGDRRTAGERGVGAAAAPRGRRRAGSPAGTLRALCAGGSQLRLPGVRAA AVPARRCHSGPAPATR (SEQ ID NO: 2412); CLASRP, Del, C_7, 81, essential, PLCSPPSPQNRSRTNGSVTTSATEAWCRTTLPASQRSSACTRSTLMSCTEASRDPAKMRRRSWQRR RLPSVIPTRTARWPR (SEQ ID NO: 2413); CDT1, Del, C_7, 81, essential, PPASRRPSWPAAPPAPPGPHSAPRPPLPVAAASAPARPPPPDATRPGHRPAGDCGCRWTRFPAPVPP RPQTSQPALLRARR (SEQ ID NO: 2414); FAM193A, Del, C_7, 82, high expression, PPSLMTSTFTSSHFKWILLLTILLRGARPVCHLQARGPAPALPSQFSSTPGSSSQTVARHQLFVVMMK MLHHCQPNLLIHH (SEQ ID NO: 2415); MIF, In, C_7, 83, high expression, PPPVHRGARGPGPAHGLRRLQRAVRALQPAQHRQDRRRAEPLLQQAAVRPAGRAPAHQPGQGLH QLLRHERGQCGLEQLHLRL (SEQ ID NO: 2416); PCNT, In, C_7, 85, essential, high expression, PPNPGCTLWPHQGPCQRPQTGSSSLPTQWGKSHSIPKFKIRKIPDCFSRSRTFLDRVYSPFRSDPAKIG RGTTRFYFKEILPPDD (SEQ ID NO: 2417); PAWR, In, C_7, 85, high expression, PPGRGQQRRRWEAPRGGSGHPGGRRCQRAQQQPPGRRAGRTCRPRSRGRELRGRLRHADAGGPRP AAVGGRAPSRLCLGCTAAPA (SEQ ID NO: 2418); SETD5, Del, C_7, 85, high expression, PPLTVLQNHPSLPRPIPAPPTLCPQTRWPHLRGHQGILAASHILETALAAIFQGGAALLVLLALPCRD PQTRQPQIQFLSPAQEL (SEQ ID NO: 2419); SYVN1, Del, C_7, 85, essential, PPGLPLQSTPLRRLPLQLLLLPPPPASLAQRPRPQPQEPPHQPLKWKGLQLLSQWAQRRCLRMESPM QQSSAGAACRSWSLLLPT (SEQ ID NO: 2420); NCOR2, In, C_7, 85, driver, PPPGGRSSHQASSPSPTATAKPAAGERRPSAAWQQPPGQEQEPGTPRRQGGREACVLPSLRSRGPEA AWGPPLLDFRPALPRAPP (SEQ ID NO: 2421); COL4A3BP, Del, C_7, 86, high expression, PPHRSGHSSLRHPPTLHPEDWAPPPAQLRERGPVSCSVVEPPCRIIRAGTRRARRRIQRRSLGRLWSA AGSSVSGQTTFMGGRIVG (SEQ ID NO: 2423); BAG6, Del, C_7, 86, essential, PPSHFLRSQWKFREQKELPLSLSGRMLPQPLEQQQKRPCPEVHLLLLRGAPGMNRMELQLRQNLGQ LQSPQNGSLLSSRTFRASGR (SEQ ID NO: 2424); NCOR2, Del, C_7, 86, driver, PPGRTQLPPSQLPQPHRHRKTCSRRATPLSSLAAAPGARAGARHPPPTRRQRSLCSSQPSQPRPRSCL GTPLAGLPACPSPCPPVR (SEQ ID NO: 2425); MYCN, Del, C_7, 86, driver, PRGRTSGRSLSCCPRPRCRPAVASRSTAPSPRAGSRRCCLRTSCGAARPRRTRSAWGDWVASPPTRS SSRTACGAASPPARSWSAP (SEQ ID NO: 2426); MUC1, In, C_7, 87, driver, PPSPRCHLGPGHQAGPGLHRPPSPWCHLGPGQQARLGLHRPSSPQCHLGLRLCIRLSFYSGAQRHLC QGYHNPSQQEHSILNSQPPL (SEQ ID NO: 2427); ZMIZ1, Del, C_7, 88, essential, PPSSPTPRTCPTTWPPSRNPSATPCRKLCHTLAALTSPTPPYNKVCTYHTPAASQGLHYITVGLLLLL LPSLPGSRHRPLPAAIHTAT (SEQ ID NO: 2428); NCOR2, Del, C_7, 88, driver, PPSRSPKSMSPPGRTQLPPSQLPQPHRHRKTCSRRATPLSSLAAAPGARAGARHPPPTRRPSQPRPRS CLGTPLAGLPACPSPCPPVR (SEQ ID NO: 2430); KMT2A, In, C_7, 89, driver, essential, PPGCGGRGGGGGKQRGWGSRGSGRRLSSLLVVRLVFVFVIVLSLFRAGPAPGGPGLRRGAAGLGR HRHQPAPVPGRVWGERRGRRQRRG (SEQ ID NO: 2431); TRIO, In, C_7, 91, essential, PPRAGLLCSLEPGRGRQDVRYVHPRALPASPWRGPRGRPQRAQQAAPRRGRRGVRARSGADPQDE GAGEPQERRRERLGVEPRRPRQGRAR (SEQ ID NO: 2432); TULP4, In, C_7, 92, high expression, PPPPLLLPLHCLHPHHQAPNSRSEQHERLCQLPISRQRAAALHHEAHRGRPGGGRPVLHALPGVPGR ACAHPQRAAHQQAAARVRHHGPADR (SEQ ID NO: 2433); USP36, Del, C_7, 92, essential, PPRAPLRRGKRPLWESRRGWAQRRASHSTSGRPLRLPTGRGRGRRRSARRTQLPAPCRRGRHRDSL GAPCTGGRARHSCPLSDGRKMAHSHR (SEQ ID NO: 2435); STATE, In, C_7, 93, driver, high expression, PPPGTEDSDQVPGWSSIPVGLEVPGGPSQASAGQGRHGDREAGAGAECASGSWGWSRKHWRNHQ QHCALGEQHSWELLLCPVQEPASQEDQAV (SEQ ID NO: 2436); AGO2, Del, C_7, 93, essential, PPPGMGRSPPLPPWWAAWTPTPIATAPPCACSSTGRRSYKTWPPWSASSSSSSTSPRASSPPASSSTA TVSLKASSSRFSTTSCWPSVRPVSS (SEQ ID NO: 2437); POLE, Del, C_7, 95, driver, essential, PPCSQRGQLGPFMNCPARNRRNTRREGWRITAGKPTRRSTSPRWKSVSPPSASGKTPSTWTPCVPSG TGVTSSKGSTRCGKRSSRRPWRWATRLR (SEQ ID NO: 2439); CIITA, In, C_7, 95, driver, PPRGPGRAGQAGLGAGPQTSKYPTGGPVPIRRREDLGDGQRLSPTPTAGRRVRAGLPQLPPAMLPG GPVAGSEWRNQGQGAPAVPSIDPKEEEAL (SEQ ID NO: 2441); SETD1A, Del, C_7, 96, essential, PPVGPLGRPSSRFHPRRRQPTACRMLYMHRGRRAEGHTHGRPTTCPCQWQPSPCPPPQSRERRPGC HPGKKQSWQRARPSRQQAPWAVCSPCWSRR (SEQ ID NO: 2443); NCOR2, Del, C_7, 96, driver, PPSRSPKSMSPPGRTQLPPSQLPQPHRHRKTCSRRATPLSSLAAAPGARAGARHPPPTRRQRSLCSSQ PSQPRPRSCLGTPLAGLPACPSPCPPVR (SEQ ID NO: 2444); SAFB, Del, G_7, 8, essential, high expression, GAMALTRG (SEQ ID NO: 2445); RPL27A, Del, G_7, 8, essential, high expression, GGPVSWWL (SEQ ID NO: 2446); AKAP9, Del, G_7, 8, driver, high expression, GGSQLSRI (SEQ ID NO: 2447); SAFB2, Del, G_7, 8, high expression, GATAPTRG (SEQ ID NO: 2448); R3HDM2, In, G_7, 8, high expression, GGGQQWDC (SEQ ID NO: 2449); WDR7, In, G_7, 8, essential, GGGLCLIR (SEQ ID NO: 2450); KAT2A, Del, G_7, 8, essential, GGIRLDLA (SEQ ID NO: 2451); NOC4L, Del, G_7, 8, essential, GGPSASWP (SEQ ID NO: 2452); RPL27A, In, G_7, 9, essential, high expression, GGGLCPGGL (SEQ ID NO: 2453); SORBS2, In, G_7, 9, high expression, GGGTVSGSV (SEQ ID NO: 2454); ANK2, In, G_7, 9, high expression, GGHEPHKRK (SEQ ID NO: 2455); SRP72, In, G_7, 10, essential, high expression, GGGVSTCAVE (SEQ ID NO: 2456); ABR, In, G_7, 10, high expression, GGGWRLPDST (SEQ ID NO: 2457); SAFB2, In, G_7, 10, high expression, GGLRLRQEAE (SEQ ID NO: 2458); SRP72, Del, G_7, 11, essential, high expression, GGCQYLRCGVK (SEQ ID NO: 2459); EPAS1, Del, G_7, 11, driver, high expression, GGTHLVAAPHI (SEQ ID NO: 2461); TSPYL2, In, G_7, 11, high expression, GGDPRILHARC (SEQ ID NO: 2462); XIRP2, In, G_7, 11, high expression, GGGCQDCEIHV (SEQ ID NO: 2463); LEF1, Del, G_7, 11, driver, GGTRNSAPRTR (SEQ ID NO: 2466); SYNPO2L, In, G_7, 12, high expression, GGGRAEETVTGV (SEQ ID NO: 2467); CCDC9, In, G_7, 12, high expression, GGPRLRAGALWP (SEQ ID NO: 2468); ZC3H3, In, G_7, 12, essential, GGPASCPAAACP (SEQ ID NO: 2470); LARP1, Del, G_7, 13, high expression, GGTAQATTPRVPR (SEQ ID NO: 2471); KAT2A, Del, G_7, 13, essential, GGPGAGGIRLDLA (SEQ ID NO: 2472); USP6, In, G_7, 13, driver, GGQPTRAGHSSGP (SEQ ID NO: 2473); CCS, Del, G_7, 14, high expression, GGLAPCRGWCASYS (SEQ ID NO: 2474); FARSA, Del, G_7, 14, essential, GDRLRSWGRRRGAS (SEQ ID NO: 2475); USP6, Del, G_7, 14, driver, GAANQSWSLLRTMR (SEQ ID NO: 2476); KIF1C, In, G_7, 15, high expression, GGELTHSHDCSPEPC (SEQ ID NO: 2477); DDB1, In, G_7, 16, essential, high expression, GGGHHHWTGVNHLSQW (SEQ ID NO: 2478); IDH2, Del, G_7, 16, driver, high expression, GGLSSGSPSSAKTSHA (SEQ ID NO: 2479); C6, In, G_7, 16, high expression, GGELFQCTNFLFLKEK (SEQ ID NO: 2480); U2AF2, In, G_7, 16, essential, GGADPGPWQPSVGCAD (SEQ ID NO: 2481); LZTR1, Del, G_7, 16, driver, GGRSGLRPWQAARGPR (SEQ ID NO: 2482); ABCA1, Del, G_7, 17, high expression, GASPTCRMWWSRQSSGC (SEQ ID NO: 2483); CCS, In, G_7, 17, high expression, GGAWHRAGGGALPTADP (SEQ ID NO: 2484); SNX27, Del, G_7, 17, high expression, GGRPIGPGCARGTASWR (SEQ ID NO: 2485); MLLT6, Del, G_7, 17, driver, GGWQAVGACPSMGSLGG (SEQ ID NO: 2486); ACACB, Del, G_7, 18, high expression, GGIGSSRRCLLPTTGLPP (SEQ ID NO: 2487); EIF3B, In, G_7, 19, essential, high expression, GGREIQANSEIQPPRGSAY (SEQ ID NO: 2488); SVIL, In, G_7, 19, high expression, GGDGGALGEAGRGRRKCAK (SEQ ID NO: 2489); FNTB, Del, G_7, 19, essential, GGFWINLASRVISTTPATA (SEQ ID NO: 2490); FOXO4, Del, G_7, 19, driver, GGFLPPVSWPRASACVPSP (SEQ ID NO: 2491); ANK2, Del, G_7, 20, high expression, GARAPQKKVRSISLTFTEIP (SEQ ID NO: 2492); DDX17, Del, G_7, 20, high expression, GEMFVLNPCLPSIMLTSHNM (SEQ ID NO: 2493); RERE, In, G_7, 20, high expression, GGVVLPTPHRPDQGGGSGRR (SEQ ID NO: 2494); DDX51, In, G_7, 20, essential, GGVREEEGAEGPAFPAKVAG (SEQ ID NO: 2495); COIL, In, G_7, 21, high expression, GGALAPRRERAPCERQRLPQS (SEQ ID NO: 2496); TSPYL2, Del, G_7, 21, high expression, GGAWKSIFRLYSRAVLVERGP (SEQ ID NO: 2497); CTBP2, In, G_7, 21, high expression, GGPAAPGAVQQLQPPKRTAPW (SEQ ID NO: 2498); LARP1, In, G_7, 22, high expression, GGGPHRQPHLACQDERRTGQGH (SEQ ID NO: 2499); GINS2, Del, G_7, 22, essential, GGTWGLLTLVYPWKCPCGWRLT (SEQ ID NO: 2500); SNX27, In, G_7, 23, high expression, GGGGRSGRGAQGGPHPGGEPREC (SEQ ID NO: 2501); FAM98B, In, G_7, 24, essential, high expression, GGWRRRLGRWWGRGRRVGGRRRRW (SEQ ID NO: 2502); ABR, Del, G_7, 24, high expression, GGMASPRLHLRDWLLGWKQGKAWR (SEQ ID NO: 2503); NOC4L, In, G_7, 24, essential, GGGPQPLGLERAVHLDSQTQPGVP (SEQ ID NO: 2504); AXIN2, Del, G_7, 24, driver, GATAGTPAPPPVPTCSPRTLRCLP (SEQ ID NO: 2505); TSPYL2, In, G_7, 25, high expression, GGEPGNRFSGCTVEQFWWRGGPRNL (SEQ ID NO: 2506); LZTR1, In, G_7, 25, driver, GGADRGCGPGRRRAVQGSPERGLRP (SEQ ID NO: 2507); EIF3B, Del, G_7, 28, essential, high expression, GERNSSKFRDSATKGFSLLTSHLVKGTW (SEQ ID NO: 2509); CCDC9, Del, G_7, 28, high expression, GAPTSSGCAVALSTSGRAAELAWAVLET (SEQ ID NO: 2510); ELL2, Del, G_7, 28, high expression, GGQGACGRSSAMGCRADGWGRTTSPYCM (SEQ ID NO: 2511); ACACB, In, G_7, 30, high expression, GGGSGHREGAYCQQRDCRREVHALHPQVGL (SEQ ID NO: 2512); LGI4, In, G_7, 30, high expression, GGLTAVGPAQSRPAPGPNADPGPAAAAAAQ (SEQ ID NO: 2513); MCL1, Del, G_7, 31, essential, high expression, GGPAWGPAAAAPPAREGDFWLRRRRPRPGER (SEQ ID NO: 2514); CDK12, Del, G_7, 31, driver, essential, GAQLSLLLMENSIGGLQESHQEGEEGEEFLT (SEQ ID NO: 2515); KAT2A, In, G_7, 31, essential, GGGSGSTWPEPAAARQSEEGASPGAAARQEA (SEQ ID NO: 2516); CDK12, In, G_7, 32, driver, essential, GGPNSVFCLWKTLSGAYKSPTKRGKRERSSLL (SEQ ID NO: 2517); ARID1A, Del, G_7, 34, driver, essential, high expression, GGTPLSISRPTSRARQSCCLPGLTHPAHQPLGSM (SEQ ID NO: 2518); KAT2A, In, G_7, 36, essential, GGGRERGGSGSTWPEPAAARQSEEGASPGAAARQEA (SEQ ID NO: 2519); MAML2, Del, G_7, 37, driver, GGPLGRGSLEGAQSPRECTVLSWSASGLGSLSAANTT (SEQ ID NO: 2520); ABCA1, In, G_7, 38, high expression, GGLRLLAGCGGAGNHQGADGHREENWCLYATDALSLLR (SEQ ID NO: 2521); EPAS1, In, G_7, 39, driver, high expression, GGGPTWWQHLTFDVETDEEPQGWELPFDAGQATERKCTQ (SEQ ID NO: 2522); LEF1, In, G_7, 40, driver, GGGPGTLRHGRDDPLQGRGRSSEGKDLRRDQSSRRGRRFS (SEQ ID NO: 2524); RNF43, Del, G_7, 41, driver, GVPPSPPLALGPRMQLCTQLARFFPITPPVWHILGPQRHTP (SEQ ID NO: 2525); R3HDM2, Del, G_7, 42, high expression, GGTTVGLLRMAATRTSLPCTPLWLCSPAPWLPKMPPFVSTTP (SEQ ID NO: 2526); PTCH1, Del, G_7, 42, driver, GGCAVLPRRTGTICTGPATATPPSLWSRFPRGRLLAGKRRCG (SEQ ID NO: 2527); AXIN2, In, G_7, 42, driver, GGQQRAPPHHPPCPPVHPGPCDASPDPTQHAGSAGGGLSQAS (SEQ ID NO: 2528); C6, Del, G_7, 44, high expression, GGALSVYQFFIPQREVKISTIILPSNKPFKPLTKRILVLLGSIK (SEQ ID NO: 2529); FYB, In, G_7, 44, high expression, GGAMGPKSGKGKGRQEFSHPETEAIASLVYLGSTSTKTQQTTKC (SEQ ID NO: 2530); TRAF3IP1, In, G_7, 45, high expression, GGEREGETERQGPRARPGQREGQGQTESEKRGALLGPGQGEEQRA (SEQ ID NO: 2531); FYB, Del, G_7, 45, high expression, GGHGAKVRKRKRETRIQPPRNRSHCLPCLPWVHLHQNPTDHQMLT (SEQ ID NO: 2532); SVIL, Del, G_7, 45, high expression, GGWWCTRGGGKRKKKMCKVSGGCTACVERCPWKGICWKWPVTVAA (SEQ ID NO: 2533); WDR7, Del, G_7, 45, essential, GGTLSHQIKSSFGQKMGKVIFTNYLPVAFQLVIHSAVMWGRQLKI (SEQ ID NO: 2534); BLM, Del, G_7, 46, driver, essential, GGLPHVERYLPKRNPPASLDPVQPHILLKRHQEPIANWGLWLHRSL (SEQ ID NO: 2536); RPS2, In, G_7, 49, essential, high expression, GGARGPWWPWDGEPRWLPRRFRQWHPGPGSRPWTGPGPRPRSSRRQGRG (SEQ ID NO: 2538); PAXS, In, G_7, 50, driver, GGGFCEWTATPGCSPPEDSGTCSSRCQALRHLQAASGQPWLCQQNSWQVL (SEQ ID NO: 2539); BAG1, In, G_7, 52, high expression, GGGAETARRPGAAGFPAARPSARPGAAPVGAPGPAWSASLWASTCPEYCQRA (SEQ ID NO: 2541); RBM33, Del, G_7, 52, essential, GAS SSPHICQRGPTHTRLSLQGSKASKEFTRRRRPSCTDEAEEWPVPWAGGA (SEQ ID NO: 2542); PTCH1, In, G_7, 52, driver, GGAAPCCRAGPGLSAPAQLLRRRLRSGADFQGEGYWPESAAVAESEVSETLI (SEQ ID NO: 2543); FAM98B, Del, G_7, 57, essential, high expression, GEEEEVVEEVSKAGEIMVEEGVMVEEGAMVEEAMEIHMEEVVVVVVVVVEEVDIEDT (SEQ ID NO: 2547); TAF15, In, G_7, 57, driver, high expression, GGLWRRPWWWQWLRWRPKWRLWRRQEWWRLWRRPRWGLRRRPRWLWRQNGRKKRLQK (SEQ ID NO: 2548); SORBS2, Del, G_7, 59, high expression, GGNRFRLCITILPGMKMSWSSEKVMSLMSWKSVMTAGLWGPQEEPNSLVLSPETTSRGC (SEQ ID NO: 2549); ZFHX3, Del, G_7, 62, driver, GGSRSSATLPGRRRRRWLRRRRQPISVAPAGPPRRPNQKPNPPGGARCVIMRPTWPGTSAFT (SEQ ID NO: 2550); RPS2, Del, G_7, 64, essential, high expression, GGPGALVALGWGTAVASAEVSAVASGAGVAAVDGAGAEAAELAEARPRIRSGCPSPSWAAWSRT (SEQ ID NO: 2551); TAF15, Del, G_7, 64, driver, high expression, GAMEETVVVAVATVETEVEAMEETGVVAAMEETEVGATEETEVAMEAKWEEETTTEMISATDHT (SEQ ID NO: 2552); DDX51, Del, G_7, 64, essential, GGSGRGRRRRSSLSCQGGWLSLTVSEGMSPKTWFLSRTSLTSILTCRSSCGHTASRPTFQSRQL (SEQ ID NO: 2554); BAG1, Del, G_7, 67, high expression, GGRGDREATGSGWVPGCAPFGQAGSRASRSPRPSVVRLPLGVHLPGVLPAGMTDPPGAPPPALAG RG (SEQ ID NO: 2555); ELL2, In, G_7, 69, high expression, GGDRGPAGGAALWAVVRTAGAGQHHRTACEAHRDGDPGARDLPEPQEFNSFSTFNPVPRTPRAC QNSQK (SEQ ID NO: 2556); HAX1, Del, G_7, 71, high expression, GGSWRVMQEVNPPNQHQTGAPRGHFIGLMMYGLWTPILEPERTMILIPRFPRRVLARFYSPSPNPIS RASL (SEQ ID NO: 2557); TRIM33, In, G_7, 72, driver, high expression, GGGGRGLLGLGPGCFISCGLSGHWSCRGRSIDAGSSSSLGSRSGSLGRAASWTASLAPGHLRRVSAE LAEPA (SEQ ID NO: 2558); FAM98B, Del, G_7, 74, essential, high expression, GVEEEVGEVVGEGEEGGGEEEEVVEEVSKAGEIMVEEGVMVEEGAMVEEAMEIHMEEVVVVVVV VVEEVDIEDT (SEQ ID NO: 2559); DDB1, Del, G_7, 75, essential, high expression, GGPSSLDRSQSPITMVTNTWLLPLLSSSKARLCATIEWTLMAQDTCWETWKAGSSCCFWRRRNRW MAPSLSRISV (SEQ ID NO: 2560); TULP4, In, G_7, 81, high expression, GGGGDTAPSAAPTCPVHLQSVQWHRAQLTARSLPGPYRQRLPVGLPVLLQPPEPTRQRPRPHRLRQ LGLHGGRGPVPALSA (SEQ ID NO: 2561); LRP1, Del, G_7, 81, high expression, GGRSTGLTGEQTHWLRPTSGPATMSPWYRGPTPSPLTCRCTTPPASPWLPIPVRPMGARAPAPTCVS STTTGPCPAPAPTS (SEQ ID NO: 2562); CTBP2, Del, G_7, 86, high expression, GARCPGRGPAASATEANCSMVTWLLWALRLLCRQHPPELETRLAVPPAHHLSTSWRRQPPILPLA WAPMPHTSPPTPGTALPPLAP (SEQ ID NO: 2563); RNF43, In, G_7, 88, driver, GGSLRAHPWLSAPGCNCAPSLPDFSPLHPQCGISLVPRGTPLDLWTSRPGQEAATRNPRPLLLKFTA SVVVPDSSPAPGTTSTWGGAF (SEQ ID NO: 2564); MCL1, In, G_7, 92, essential, high expression, GGGRLGGRQRRRHPPGRATFGYGEGGLGPARDRGRGGRRGDWRKRRRKPPVHPHARLPEGRAAA AHWRRGPRRHRDPREAAFLRAHPPRGAA (SEQ ID NO: 2565); PTPRB, In, U_7, 8, driver, high expression, FFFFTHHY (SEQ ID NO: 2566); WDFY3, In, U_7, 8, high expression, FFAQFGPS (SEQ ID NO: 2567); ARFGAP3, In, U_7, 8, high expression, FFCLSRFS (SEQ ID NO: 2568); FNIP1, In, U_7, 8, high expression, FFFTFSSL (SEQ ID NO: 2569); UBTD2, In, U_7, 8, high expression, FFFWQTSH (SEQ ID NO: 2570); EDNRB, In, U_7, 8, high expression, FFLYTNDL (SEQ ID NO: 2571); LRP2, Del, U_7, 8, high expression, FFQICQNT (SEQ ID NO: 2572); PPP1CC, In, U_7, 8, high expression, FFSSQREP (SEQ ID NO: 2573); DENND4A, Del, U_7, 8, high expression, FLMHSGSF (SEQ ID NO: 2574); ATRN, Del, U_7, 8, high expression, FLVMLLII (SEQ ID NO: 2575); DST, Del, U_7, 8, high expression, FWQNMETK (SEQ ID NO: 2576); NCAPG2, In, U_7, 8, essential, FFLCFITS (SEQ ID NO: 2577); NOL8, Del, U_7, 8, essential, FLIQTLKT (SEQ ID NO: 2578); ELK4, Del, U_7, 8, driver, FFHRHPSY (SEQ ID NO: 2579); KIAA0100, In, U_7, 9, essential, high expression, FFLDPECQS (SEQ ID NO: 2580); ATP8B1, Del, U_7, 9, high expression, FFHPVETVP (SEQ ID NO: 2581); ECM2, In, U_7, 9, high expression, FFSAYHFSN (SEQ ID NO: 2582); TNFSF10, In, U_7, 9, high expression, FFWGLFSWL (SEQ ID NO: 2583); ZMPSTE24, Del, U_7, 9, high expression, FLFMPGCSH (SEQ ID NO: 2584); ARHGAPS, Del, U_7, 9, high expression, FLKMRLSKS (SEQ ID NO: 2585); SLC7A2, Del, U_7, 9, high expression, FLLCALYYF (SEQ ID NO: 2586); ARFGAP3, Del, U_7, 9, high expression, FLPLTFLLR (SEQ ID NO: 2587); TSPAN6, Del, U_7, 9, high expression, FWSNWSLPS (SEQ ID NO: 2588); KNTC1, Del, U_7, 9, essential, FFVLQTFSF (SEQ ID NO: 2589); XRCC2, In, U_7, 9, essential, FFYYWRKWG (SEQ ID NO: 2590); POLE2, Del, U_7, 9, essential, FLEVLLIHL (SEQ ID NO: 2591); SEC16A, Del, U_7, 9, essential, FLSKSILLP (SEQ ID NO: 2592); CUL2, In, U_7, 10, essential, high expression, FFGKSCSAFA (SEQ ID NO: 2593); SMC3, In, U_7, 10, essential, high expression, FFLCNSVCSQ (SEQ ID NO: 2594); MPHOSPH10, Del, U_7, 10, essential, high expression, FLIQLKVMKT (SEQ ID NO: 2595); ABCE1, Del, U_7, 10, essential, high expression, FWMAMFQQKT (SEQ ID NO: 2596); CHD9, In, U_7, 10, high expression, FFCRHGRRTI (SEQ ID NO: 2597); GRSF1, In, U_7, 10, high expression, FFFRLQNPQR (SEQ ID NO: 2598); AKAP11, In, U_7, 10, high expression, FFFVFSCLLI (SEQ ID NO: 2599); DMD, In, U_7, 10, high expression, FFFWSSCKRP (SEQ ID NO: 2600); UBTD2, Del, U_7, 10, high expression, FFLADLSLTK (SEQ ID NO: 2601); FILIP1, Del, U_7, 10, high expression, FFLVPLSFLP (SEQ ID NO: 2602); SETX, In, U_7, 10, high expression, FFPFDGIEYF (SEQ ID NO: 2603); IBTK, In, U_7, 10, high expression, FFSEIVSFRW (SEQ ID NO: 2604); GCC2, In, U_7, 10, high expression, FFVSKRRCYP (SEQ ID NO: 2605); SYNE2, Del, U_7, 10, high expression, FSRSLLLTCC (SEQ ID NO: 2606); WDFY3, Del, U_7, 10, high expression, FYYWECSAAC (SEQ ID NO: 2607); NCAPG2, Del, U_7, 10, essential, FFMFHYFLTF (SEQ ID NO: 2608); SMG1, Del, U_7, 10, essential, FLMMIITGRC (SEQ ID NO: 2609); BDP1, In, U_7, 11, essential, high expression, FFCSFASESSC (SEQ ID NO: 2610); PPP2R2A, Del, U_7, 11, essential, high expression, FFPKSSPLFRM (SEQ ID NO: 2611); CNOT1, Del, U_7, 11, essential, high expression, FFSPILIKSYI (SEQ ID NO: 2612); PRPF8, Del, U_7, 11, essential, high expression, FSGSQQMRLGT (SEQ ID NO: 2613); MSH6, Del, U_7, 11, driver, high expression, FLEMILFLMTF (SEQ ID NO: 2614); DST, In, U_7, 11, high expression, FFGKTWRQNDR (SEQ ID NO: 2616); FNIP1, Del, U_7, 11, high expression, FFHIFLSLKAT (SEQ ID NO: 2617); ARMCX3, In, U_7, 11, high expression, FFLFKRISSVC (SEQ ID NO: 2619); ARMCX1, In, U_7, 11, high expression, FFLIQRVWSLC (SEQ ID NO: 2620); AASS, In, U_7, 11, high expression, FFSRIKFGRLS (SEQ ID NO: 2621); UBE3C, Del, U_7, 11, high expression, FFTNKMKTQNV (SEQ ID NO: 2622); LEPROTL1, Del, U_7, 11, high expression, FFTSFHLFHTA (SEQ ID NO: 2623); ANKRD12, Del, U_7, 11, high expression, FLKVMKLKISF (SEQ ID NO: 2624); SLC5A3, Del, U_7, 11, high expression, FLPCGNLIEAP (SEQ ID NO: 2625); MYSM1, Del, U_7, 11, high expression, FLVKKSRSHFR (SEQ ID NO: 2626); MFN2, Del, U_7, 11, essential, FLAGRAMGRAP (SEQ ID NO: 2628); RBM12, Del, U_7, 12, essential, high expression, FFMGSVLMQCIC (SEQ ID NO: 2629); CCT6A, Del, U_7, 12, essential, high expression, FFTRVQKREKNS (SEQ ID NO: 2630); BAZ1A, In, U_7, 12, high expression, FFLPDCNLPGNS (SEQ ID NO: 2632); USPL1, In, U_7, 12, essential, FFFLLSIKCFSK (SEQ ID NO: 2633); BTAF1, In, U_7, 12, essential, FFGEGSSVAGSN (SEQ ID NO: 2634); NAA25, In, U_7, 12, essential, FFQLGSKRQGRF (SEQ ID NO: 2635); SPRTN, In, U_7, 12, essential, FFYQERANKKQW (SEQ ID NO: 2636); XRCC2, Del, U_7, 12, essential, FLLLEKVGLNFV (SEQ ID NO: 2637); ABCB7, Del, U_7, 12, essential, FLTKFEMQYLAR (SEQ ID NO: 2638); XPOS, Del, U_7, 12, essential, FWKVLSPRCFEH (SEQ ID NO: 2639); EIF3E, Del, U_7, 13, driver, essential, high expression, FLECWFQQQIEML (SEQ ID NO: 2640); RBM12, Del, U_7, 13, essential, high expression, FLKSWILWKIVFI (SEQ ID NO: 2641); HECTD1, Del, U_7, 13, essential, high expression, FLNLCVWVTLKAI (SEQ ID NO: 2642); TMPO, Del, U_7, 13, high expression, FCFWSIKLWKPTK (SEQ ID NO: 2643); SOAT1, In, U_7, 13, high expression, FFCLFALLAQCLC (SEQ ID NO: 2644); SLC35F5, In, U_7, 13, high expression, FFFIFYCNSPMPL (SEQ ID NO: 2645); VPS13B, In, U_7, 13, high expression, FFKEDKKCTQFGT (SEQ ID NO: 2646); ROCK2, In, U_7, 13, high expression, FFLGRKRYYGLCQ (SEQ ID NO: 2647); PDS5B, Del, U_7, 13, high expression, FFSSILIKMFAYW (SEQ ID NO: 2648); CLPX, Del, U_7, 13, high expression, FLLCYLKQTQRKA (SEQ ID NO: 2649); SYNE2, Del, U_7, 13, high expression, FLSFQKKLMKNPP (SEQ ID NO: 2650); RIF1, In, U_7, 14, essential, high expression, FFFQTCKYTYHCCS (SEQ ID NO: 2651); HIPK3, Del, U_7, 14, high expression, FFAKKQICLILVGD (SEQ ID NO: 2652); HNRNPH2, In, U_7, 14, high expression, FFEFYSRGKWWRLW (SEQ ID NO: 2653); SYNE1, Del, U_7, 14, high expression, FFEIYLIGFSSILP (SEQ ID NO: 2654); BRWD1, In, U_7, 14, high expression, FFKGVQILERKCTI (SEQ ID NO: 2655); ZCCHC2, In, U_7, 14, high expression, FFSASDSICKWTGT (SEQ ID NO: 2656); PANK2, In, U_7, 14, high expression, FFWSLLSSYWLYHF (SEQ ID NO: 2657); DHRS7, Del, U_7, 14, high expression, FLMAFEQNLPHTQV (SEQ ID NO: 2658); BTBD1, Del, U_7, 14, high expression, FSFLVPLAIIMALQ (SEQ ID NO: 2659); PCCA, Del, U_7, 14, high expression, FWMRRLEERWENKL (SEQ ID NO: 2660); XPOS, In, U_7, 14, essential, FFGKCYHPDVSNTK (SEQ ID NO: 2661); ALG13, In, U_7, 14, essential, FFSSPSYPHPVQNA (SEQ ID NO: 2662); CNOT1, In, U_7, 15, essential, high expression, FFLCLYMALGVTTYS (SEQ ID NO: 2663); CCT6A, In, U_7, 15, essential, high expression, FFLQECRRERKTRES (SEQ ID NO: 2664); PHF3, Del, U_7, 15, high expression, FFGTQMLNIRTNIEV (SEQ ID NO: 2666); NNT, In, U_7, 15, high expression, FFYWSDHSCWKSSSS (SEQ ID NO: 2667); MGST1, Del, U_7, 15, high expression, FLLDMELLFPWLTGC (SEQ ID NO: 2668); UBR1, Del, U_7, 15, high expression, FLWRWNTKNSLLWNL (SEQ ID NO: 2669); PPP2R2A, In, U_7, 16, essential, high expression, FFFRNHLLYFGCKIQP (SEQ ID NO: 2670); GCC2, Del, U_7, 16, high expression, FCLKKKMLSLKNILLN (SEQ ID NO: 2671); DMXL2, In, U_7, 16, high expression, FFFRYTNTGAPRSKFL (SEQ ID NO: 2672); TSPAN6, In, U_7, 16, high expression, FFGRTGRCHRRICFQT (SEQ ID NO: 2673); SOS2, In, U_7, 16, high expression, FFWNIFNKYSEDRRRE (SEQ ID NO: 2674); CHD9, Del, U_7, 16, high expression, FLQTWKKNHLTQTTLK (SEQ ID NO: 2675); ARMCX1, Del, U_7, 16, high expression, FSYSKSLEFVLRKSKH (SEQ ID NO: 2676); KNTC1, In, U_7, 16, essential, FFLYYKPSAFKNSTSN (SEQ ID NO: 2677); COX11, Del, U_7, 16, essential, FLKQRKGTSCQFQDII (SEQ ID NO: 2678); XRCC6, Del, U_7, 17, essential, high expression, FWLMPPRLCLNLRVKMS (SEQ ID NO: 2679); ATM, Del, U_7, 17, driver, high expression, FFPRLFSVRDKKRALQV (SEQ ID NO: 2680); ATP13A3, In, U_7, 17, high expression, FFIYFYIIHHVVSSCLC (SEQ ID NO: 2681); KIFAP3, In, U_7, 17, high expression, FFLFLQLFSISWTYYSL (SEQ ID NO: 2682); GRSF1, Del, U_7, 17, high expression, FFQTAESATVRMEYIFS (SEQ ID NO: 2683); PHIP, In, U_7, 17, high expression, FFWIRSARENIRIGVSY (SEQ ID NO: 2684); HELZ, Del, U_7, 17, high expression, FLIVQLLIGHSLLLQKL (SEQ ID NO: 2685); NNT, Del, U_7, 17, high expression, FLLVRSQLLEKFLQLRF (SEQ ID NO: 2686); SLC35F5, Del, U_7, 17, high expression, FLQMLKVTLLLAQQIQL (SEQ ID NO: 2687); GATB, Del, U_7, 17, essential, FLMHLYLELCRFSTGGV (SEQ ID NO: 2688); MGST1, In, U_7, 18, high expression, FFCWIWSYSFHGLQVAEK (SEQ ID NO: 2689); TMTC1, In, U_7, 18, high expression, FFHKRKPIKRAEPSRQSF (SEQ ID NO: 2690); C4BPB, In, U_7, 18, high expression, FFLVCVLSYGCVASFCFR (SEQ ID NO: 2692); ATP8B1, In, U_7, 18, high expression, FFSTRWKPCLNHHWFLVE (SEQ ID NO: 2693); TMPO, In, U_7, 18, high expression, FFVFGLSSYGNQPSKSLL (SEQ ID NO: 2694); FAM13B, Del, U_7, 18, high expression, FLKKSRRKKKMVLICPLS (SEQ ID NO: 2695); C11orf54, Del, U_7, 18, high expression, FLVVMEKVDTTIMTLLQI (SEQ ID NO: 2696); MYC, In, U_7, 18, driver, essential, FFSGSGKPAASRDDAPQR (SEQ ID NO: 2697); COPS2, Del, U_7, 19, essential, high expression, FLKPSRIMMNLEVQDEPLA (SEQ ID NO: 2699); PTPRB, Del, U_7, 19, driver, high expression, FFLYPSLLNQSPCLELLKV (SEQ ID NO: 2700); TRIP12, In, U_7, 19, high expression, FFFFSTSWRRAHWKSGTSG (SEQ ID NO: 2701); COBLL1, Del, U_7, 19, high expression, FFNAVRKSETKLQVPLQPL (SEQ ID NO: 2702); UBR1, In, U_7, 19, high expression, FFYGDGIQKTLCYGICEVL (SEQ ID NO: 2703); FANCD2, Del, U_7, 19, driver, FLKTRTVMKSTYLDSLSVN (SEQ ID NO: 2705); ABCE1, In, U_7, 20, essential, high expression, FFGWLCSNRKLEIQRCITCF (SEQ ID NO: 2706); SPARCL1, In, U_7, 20, high expression, FFPMSLGNCSCNPDKCKIII (SEQ ID NO: 2707); MIPEP, Del, U_7, 20, essential, FFSEQTNHIRIAISLSVEAD (SEQ ID NO: 2708); MED23, Del, U_7, 20, essential, FLQALIQFRELGVKTYFRPS (SEQ ID NO: 2710); ERG, In, U_7, 20, driver, FFCCPKPILEFTNWGYIPQH (SEQ ID NO: 2711); CNOT1, Del, U_7, 21, essential, high expression, FSLSIYGTGRYHLFLIITIFV (SEQ ID NO: 2712); HIPK3, In, U_7, 21, high expression, FFLQRNRYVSFWLEIKDIGRA (SEQ ID NO: 2713); VPS13A, Del, U_7, 21, high expression, FFMNLTREPSRVLKSLWKEWH (SEQ ID NO: 2714); ZMPSTE24, In, U_7, 21, high expression, FFYLCLAVHISCVSGSCHNLC (SEQ ID NO: 2715); DMXL2, Del, U_7, 22, high expression, FFQIHKYRSTKIQIPIAGLFYI (SEQ ID NO: 2716); ATP13A3, Del, U_7, 22, high expression, FYIFLYYSSCCIQLPLLTRFFR (SEQ ID NO: 2717); CENPE, Del, U_7, 22, essential, FLIADQSLYHHLIQFAILITQV (SEQ ID NO: 2718); ELK4, In, U_7, 22, driver, FFFTGSLLALYGVTIAFIYLPF (SEQ ID NO: 2719); NAIP, Del, U_7, 23, high expression, FFHVALSLMMMISQKQGLMKMKI (SEQ ID NO: 2720); SYNE1, In, U_7, 23, high expression, FFSKFTSLDSAAYCHERCWQFSN (SEQ ID NO: 2721); KIFAP3, Del, U_7, 23, high expression, FFVSPAFLNFMDLLLTIKLELCV (SEQ ID NO: 2722); TTN, Del, U_7, 23, high expression, FLNFRWWKGLPGLSKVFLTVMHQ (SEQ ID NO: 2723); NUP214, Del, U_7, 23, driver, essential, FLMFAHSQMRLNSKNAHLPIISF (SEQ ID NO: 2724); KNTC1, In, U_7, 23, essential, FFTAKRRKPCLCTVSLASTSGKL (SEQ ID NO: 2725); COQ2, In, U_7, 24, essential, high expression, FFRDCPWEFVERKEDRQNKEGYRE (SEQ ID NO: 2727); ATP13A3, In, U_7, 24, high expression, FFCFFCDFFIYFYIIHHVVSSCLC (SEQ ID NO: 2728); CTDSPL2, Del, U_7, 24, essential, FFHQQIKMERQDQILQDRLWKLKK (SEQ ID NO: 2729); BDP1, Del, U_7, 25, essential, high expression, FLLICFRKFLLKKRKENKNLLKITV (SEQ ID NO: 2730); SLC7A2, In, U_7, 25, high expression, FFCCVPYITSSRSFVFWSKRVCLGE (SEQ ID NO: 2731); TRIP12, Del, U_7, 25, high expression, FFLLHFLEKSPLEEWNQWVMHLCWH (SEQ ID NO: 2732); ALG13, Del, U_7, 25, essential, FFLSLLPPPCTKCIKDGKTTAARSL (SEQ ID NO: 2733); BCAP29, In, U_7, 26, high expression, FFLASFETSGYAYYSTGKRTVKQRCT (SEQ ID NO: 2734); INTS2, Del, U_7, 26, essential, FLCQLCQVWSLFVEHFLHCMRILCLC (SEQ ID NO: 2735); ROS1, In, U_7, 26, driver, FFRYGSKSSCVDVFSRKSYQCRLLHS (SEQ ID NO: 2736); KIAA1429, In, U_7, 27, essential, high expression, FFFWTHTPFRAAASSHHANNSGITSI (SEQ ID NO: 2737); CEPT1, Del, U_7, 27, high expression, FFVVLRGHLCSIVRTGKRMFLEHCDLE (SEQ ID NO: 2738); OCIAD2, Del, U_7, 27, high expression, FLKISSVGLVLVHSITGTASLPVRNAK (SEQ ID NO: 2739); BTAF1, Del, U_7, 27, essential, FWRRFFRGWEQLMTVSNKRVQLKHLPV (SEQ ID NO: 2740); RBPJ, In, U_7, 28, essential, high expression, FFLPTSLCISYGQWMEEKKRTNGTRWLF (SEQ ID NO: 2741); MPHOSPH10, Del, U_7, 28, essential, high expression, FLKILILMKMKGDCLEVKNLSQVKVPEI (SEQ ID NO: 2742); EVI2B, In, U_7, 28, high expression, FFFKDRDNYNREAVTAYLIHIINVTGIG (SEQ ID NO: 2743); TCF4, In, U_7, 28, high expression, FFPVYHSQSEEVLFPLLLAYIASSAYFT (SEQ ID NO: 2744); ANK2, Del, U_7, 28, high expression, FLPSKKIDFLYLSRYAIRLRNLADDYHL (SEQ ID NO: 2745); NUFIP1, Del, U_7, 28, essential, FFVIPVIVVLKIKKSMTNTCLNIQNALN (SEQ ID NO: 2746); GABPA, Del, U_7, 29, essential, high expression, FFSGFLGEKFSGVIWNFSENMYWQVKNNR (SEQ ID NO: 2747); ECM2, Del, U_7, 29, high expression, FFCLSFFKLTLEKMKKFLGSKGGRSTTEG (SEQ ID NO: 2748); CEPT1, In, U_7, 29, high expression, FFLLFCGDIYVLLCALANVCFWNIAIWNN (SEQ ID NO: 2749); ALDH1A1, Del, U_7, 29, high expression, FLHIQDMNLLVYVAKSFLGISRWLCSFGR (SEQ ID NO: 2750); SPARCL1, Del, U_7, 29, high expression, FSYVSWELQLQSRQMQDYYLIIPNQLLKR (SEQ ID NO: 2751); CENPC, In, U_7, 29, essential, FFFYWEIDIRTTRRKGKAACWPGYIGFLC (SEQ ID NO: 2752); CUL4B, Del, U_7, 30, high expression, FCFWIELTFFRIQCYPPFGTWDWSYLGLIL (SEQ ID NO: 2755); C4BPB, In, U_7, 30, high expression, FFLVCVLSYGCVASFCFRCRALSRASSSGQ (SEQ ID NO: 2756); KIAA1429, Del, U_7, 31, essential, high expression, FFLDSYSFQSCCLFHCPCKQLRYHFHITCIL (SEQ ID NO: 2758); GABPA, In, U_7, 31, essential, high expression, FFSAGSSGRNSLESSGTSPKICIGKSRTTDE (SEQ ID NO: 2759); CCT7, In, U_7, 31, essential, high expression, FFYWLPQGQDMHLHSPWRRRAVYGGDRAVPA (SEQ ID NO: 2760); BCAP29, In, U_7, 31, high expression, FFNATHRLYCNGIIKIFDDVSDILQSLYSSS (SEQ ID NO: 2761); AKAP11, Del, U_7, 31, high expression, FFRLLLPTHLNQNVQVQVLLFSWMKRDIKKA (SEQ ID NO: 2762); ROCK2, Del, U_7, 32, high expression, FFGKKEILWPLPIAPGWFSFFMPFKMIGICTW (SEQ ID NO: 2764); PHIP, Del, U_7, 32, high expression, FLDQVSQRKYQNWSFILTKLTVSSFPTLVTGL (SEQ ID NO: 2766); DDX10, Del, U_7, 33, driver, essential, high expression, FFPVAKRSSICTECFAGYVLVFLSLHSMVDSSK (SEQ ID NO: 2767); NUP205, Del, U_7, 34, essential, high expression, FLIGPRLNKLLLTVNTRIYGDRQSAMSSFFIGFL (SEQ ID NO: 2768); CCT7, Del, U_7, 34, essential, high expression, FLLAAPRPRHAPSFSVAAPSSLWRRQSGPCMMPS (SEQ ID NO: 2769); MTA2, Del, U_7, 34, essential, FFTHWCLTPCRRHFSLIRARLELVANTKLRSQIA (SEQ ID NO: 2770); RIF1, Del, U_7, 35, essential, high expression, FFPNMQIHLSLLFMIALLQLEKMPPMWLSVLSGRS (SEQ ID NO: 2771); SNX14, In, U_7, 35, high expression, FFCICLNKKDSQGGYSIYYNQETIKSSNEAYRSDS (SEQ ID NO: 2772); PCCA, In, U_7, 35, high expression, FFGCGDSKSDGRTSCSSCQSSKIFLCWDRGVPCGL (SEQ ID NO: 2773); DMD, Del, U_7, 35, high expression, FFLVELQKAIKCTIPWWNIALRLHQEKMFETLPRY (SEQ ID NO: 2774); ERG, Del, U_7, 35, driver, FLLPQTHTGIHQLGVYTPTLGSPPAICLLIWALTT (SEQ ID NO: 2775); RANBP2, Del, U_7, 36, driver, essential, high expression, FFVESVVILMKTMEMGRTFNQSFKKFRKLKNLRQKK (SEQ ID NO: 2776); ALG11, Del, U_7, 36, essential, high expression, FHHYSSLGSLYVELYVCVWSLSFGESDCCYRERKN (SEQ ID NO: 2777); RNF34, In, U_7, 36, high expression, FFFKLYSPLCYYVFVSGRAYGWRPNIQIWSAGTGTK (SEQ ID NO: 2778); CCNE1, Del, U_7, 36, driver, FCRIQMKKWPKSTGRRGTSVGASLGTIMQSVQTPAP (SEQ ID NO: 2779); FAS, Del, U_7, 36, driver, FFVTLLYVNTVTLAPNVNMESSRNAHSPATPSAKRK (SEQ ID NO: 2780); ALG11, In, U_7, 38, essential, high expression, FFKETLSCGRFTVSSLHTAGPKSRIHFSWLGSSNAVCS (SEQ ID NO: 2781); SON, In, U_7, 38, essential, high expression, FFQSEYCCSKTNSTKKPGNINKRIPCIIWISTSEKRSG (SEQ ID NO: 2782); PYGL, Del, U_7, 38, high expression, FFLPSSLTSSKISSTCYFIMTGLKSLQTTKPMSSVKIK (SEQ ID NO: 2783); ELK4, In, U_7, 38, driver, FFFTDTHHTDSKPLALQYPLLEYSQSCCSPKSSQTARC (SEQ ID NO: 2784); RBPJ, Del, U_7, 39, essential, high expression, FFAHLLVYILWAVDGRKKKNKWNAMVVLNKSLNRVHLLG (SEQ ID NO: 2785); ALG11, In, U_7, 40, essential, high expression, FFLFIILPWAHCMWNFMCVFGHCPLGNQTAATEKEKISVN (SEQ ID NO: 2788); SMCS, Del, U_7, 40, essential, high expression, FLVPCSVLVKLISIQKMRKIMINMEFELESNFEVVLNCMN (SEQ ID NO: 2789); SERINC5, Del, U_7, 40, high expression, FLKICVKALKLVTPVRSWWDILPCIESVLEWLVSSLSSVY (SEQ ID NO: 2790); SLC12A1, Del, U_7, 40, high expression, FSQQLLGFLLVPISQEIWRIPKMPSPEEPCWPFSSPLLPT (SEQ ID NO: 2791); HNRNPH1, Del, U_7, 41, essential, high expression, FFLTAKFKMGLKVFVSSTPEKADQVARLLLNLNQKMKSNWP (SEQ ID NO: 2792); ATM, In, U_7, 41, driver, high expression, FFFQGYSVCETRKELFRSKSYLSSSYYLPQDFGCQLSNSSV (SEQ ID NO: 2793); WDFY3, Del, U_7, 41, high expression, FCPVWPLIMLLQIIPSQQVLLMGLWSVALVLVKDSSLLPPA (SEQ ID NO: 2794); MFN2, In, U_7, 42, essential, FFWPDEQWEEHRDQCHALGQSSALWDWPHHQLLPAGRGHRWP (SEQ ID NO: 2795); KIAA1549, In, U_7, 42, driver, FFKLPVFNATAVRRRHYYTVALLGGGLTASRGGLGHKCRQIH (SEQ ID NO: 2796); ZCCHC2, Del, U_7, 43, high expression, FFCLRLHMQMDWYMTQSWGAKPTMACSRWQDLGDSILYIQHLT (SEQ ID NO: 2797); PPP1CC, Del, U_7, 43, high expression, FFFSEGTMNVPASTEFMDFMMNVKEDTTLNYGKLSQTVLTVYR (SEQ ID NO: 2798); TMEM87A, In, U_7, 43, high expression, FFHGDVYCICPVWCSVAGMVCLLLERSPENSVLDWCCHLPGNA (SEQ ID NO: 2799); DHX9, In, U_7, 44, essential, high expression, FFAWLESDLYYAEAFGNESTFWKPSVSDSTPAFSDSSRGTAQSV (SEQ ID NO: 2800); TSPAN13, In, U_7, 45, high expression, FFLYDYSVTCIYCSVFCILRLFSPEPGATGSASGGWLEQYGKCSK (SEQ ID NO: 2801); TNPO1, Del, U_7, 46, essential, high expression, FFVMPLHHGLTQKMISETCSVRSFMDLKIKLAMKIGGVSLTSFLFP (SEQ ID NO: 2803); CENPP, Del, U_7, 46, essential, FLWSGLNIVSARLNISRKSTQMPCTSRRGPPPAPWGSAAPAGQGLN (SEQ ID NO: 2804); UPF3B, Del, U_7, 48, high expression, FFLMIRVCILICMPEHTSTLKTKRTLFCSGIALMVMYSLTIKVRNIPL (SEQ ID NO: 2805); MIPEP, In, U_7, 51, essential, FFSASRQTTSGLPFHYPWRQTKGRWRLSTPSCSSYAESSPFLKEFSNFANS (SEQ ID NO: 2806); FAS, Del, U_7, 53, driver, FFVTLLYVNTVTLAPNVNMESSRNAHSPATPSAKRKDPDLTWGGFVFFFCQFH (SEQ ID NO: 2811); CUL2, Del, U_7, 54, essential, high expression, FWKIMFGICIREFWSQKNKYLLCIIGTGKNTARVQTIWTAYIGISTPSLLKRIN (SEQ ID NO: 2812); C4BPB, Del, U_7, 54, high expression, FFGVRAVLWLRGEFLLQMQSTVQSFLQWTIAYLSQRRWKDRFWGLTFVSRATTW (SEQ ID NO: 2813); DENND4C, Del, U_7, 55, high expression, FLKLLGNFLCLSTNFLCLDHILFPLKSTFHILCKTSLFLHHKDRESLSSCQSMMH (SEQ ID NO: 2814); SLC12A1, In, U_7, 60, high expression, FFSPSYWDSCWCQYLRRFGGSPRCHPQRNHAGHFHHHCCLLRGCNLCRGLCGPRCHREHE (SEQ ID NO: 2815); TBCC, In, U_7, 60, essential, FFSPRLRPAAGTRGAGAAAGGLGRAAPGAAAQEAFRFQDPGKGCCFVYQSRRGSWHPPGS (SEQ ID NO: 2816); TM9SF3, In, U_7, 61, high expression, FFCYSSSKSCWYNTWPKSVRSAQLSLSCQCCASSYTGEKMVHGACGYCLPGWNFTFWFNLY (SEQ ID NO: 2817); TPR, Del, U_7, 65, driver, essential, high expression, FLMMKTEQFQVLQLLWCHIVLMDLLKQFIRRRLLVSLDSGLGHLKICHKQVLVTLILASLLLKEV (SEQ ID NO: 2818); SOAT1, Del, U_7, 69, high expression, FLPFCTAGSMPLLRCYALVTGCSIRIGGTPRHTPTIIEPGMWWSMTGYITMLTRTFSGFSPRDSNLLP C (SEQ ID NO: 2819); STK38, Del, U_7, 70, high expression, FLKALTGNISERDLLQYLLKSKALMIPQTSMSFQNLIFLSQQWPQVIILRLTTRTKTGSSSITRTSALR A (SEQ ID NO: 2821); ZMYM4, Del, U_7, 71, high expression, FLIKQLIKLKKHYIPIYHKPQKQTLGIPATHLPIKNPLVRNWGIPLHQILELKKNLWMMSMTKQWHH SRDY (SEQ ID NO: 2822); SENP6, Del, U_7, 72, essential, high expression, FLRKFPLKKLMADLLPVQEPMKRASKEVVGKRKTKLKLYHLNLKYNLEANKNFSFLMKKKKLEK TTPSSLAQ (SEQ ID NO: 2823); RNF34, Del, U_7, 72, high expression, FFQTIQPPLLLCLRFRESLWMETKHPDLECRHRYKVKSLQQTQKMMMTTMMRMMMMKKKTQRI GTPGSPRRE (SEQ ID NO: 2824); KIAA0100, Del, U_7, 78, essential, high expression, FFGSRMSVLSFSNTSKQWKLITCGFPANSLAMIFHTMWHCALEKCVSERTYRKFLTCLPHSPRALG WIKRNCPSAHPY (SEQ ID NO: 2825); UNC50, In, U_7, 78, high expression, FFHQPCYPDRHIYWIFSWKYLMVGCSWLLYLCNFPGIQCIAIFEKYSNSSVSICTSDSALRAFPGTGM ELHPYSLFFL (SEQ ID NO: 2826); ASNSD1, Del, U_7, 78, high expression, FLVAVACFGILVIWARVSASLQLAPKHLDWQISGKKFQHLDFSELILSLLSFPDALFYNCILGNIFLG RILLKKMLIA (SEQ ID NO: 2827); ICE1, Del, U_7, 82, essential, FLMKIFKLQLTSSNFPLLFCHQCPRPLRCHHLTRVPYRLHLHLKPTLENTQIPAIMTQSSLEILLSVFQ KMIQLNHRIILAH (SEQ ID NO: 2828); EVI2B, Del, U_7, 93, high expression, FFQRQRQLQQRSSHSLPYSHHQCHRYWLILKTQQGILWVNQHNSATLFLDNQYHLPKSLLDNQHQ LSIPLLKNQKHILLLDNHLPTTPNNQHQ (SEQ ID NO: 2829).

The following peptides are all MS FSs in dogs meeting the criteria of >6 homopolymers, a FS peptide length >7 amino acids long, and at least one of a cancer driver gene, an essential gene, or a highly expressed gene. XM_005615627.2, ECHDC1, A_10, In, 9, high expression, TSTVSWWIR, (SEQ ID NO: 2830); XM_014116982.1, TTF1, A_10, Del, 12, essential, KERISSIFFLPL, (SEQ ID NO: 2831); XM 014110220.1, KIAA1524, A_10, Del, 12, essential, NVKGWSRPLKFC, (SEQ ID NO: 2832); XM 014116982.1, TTF1, A_10, In, 18, essential, KKGFPASSFFPFEKIRNM, (SEQ ID NO: 2833); XM 005623762.2, DDX24, A_10, In, 20, essential, high expression, IGAFPGYCSKGAQKSKDMDA, (SEQ ID NO: 2834); XM_536376.4, HK1, A_10, Del, 21, high expression, KSRTRNYQWDSHFLSLVGSPR, (SEQ ID NO: 2835); XM_532252.6, SEC63, A_10, In, 23, essential, high expression, TYTCALTTVKATETKAGKWNCWE, (SEQ ID NO: 2836); XM_005615627.2, ECHDC1, A_10, Del, 23, high expression, NFNSFLVDPLTFRRKTVALVFLP, (SEQ ID NO: 2837); XM 005623082.2, ANKRD12, A_10, Del, 24, high expression, KTKQQTVVTTLSQKKVKIKKRTGK, (SEQ ID NO: 2838); XM_532252.6, SEC63, A_10, Del, 27, essential, high expression, NLHLCPYHSQSNRNKSRQMELLGVKLR, (SEQ ID NO: 2839); XM_005623762.2, DDX24, A_10, Del, 29, essential, high expression, NWSLPRVLLQRCPKKQRHGCLKCMTIRQM, (SEQ ID NO: 2840); XM 536376.4, HK1, A_10, In, 33, high expression, NQGQEITSGIHIFFPLSAVQDRRGHPDHLDKEI, (SEQ ID NO: 2841); XM_014117603.1, ZNF207, A_10, Del, 56, essential, high expression, KRSNKMILMNTMMTLQLQLHFSHSLFSLNKVISPRWCNQDCHQFQEHQECLQAYLH, (SEQ ID NO: 2842); XM_014119593.1, EIF2B3, A_10, Del, 57, essential, KQWSSVTSLEWTAQERGCSSWLMKRTWMKSWSLRDPSCRSTQEYISTQALWTPISTA, (SEQ ID NO: 2843); XM_536179.5, CHD2, A_11, In, 12, high expression, ILLNLSSESCTF, (SEQ ID NO: 2844); XM 014118224.1, MARCKS, A_11, Del, 12, high expression, KRSASPSRSPSS, (SEQ ID NO: 2845); XM 005640067.2, E2F3, A_11, Del, 18, essential, KHGMTHHLVCSPRSSFSC, (SEQ ID NO: 2846); XM 005636122.2, SBNO1, A_11, In, 23, essential, high expression, KEKYRSRFYSKCLISIRSWIKTT, (SEQ ID NO: 2847); XM_005615796.1, ZCCHC6, A_11, In, 24, high expression, NNTRELWEYPQKRTICCFQQSLCV, (SEQ ID NO: 2848); XM_846664.4, SEC62, A_11, Del, 58, essential, high expression, KMGKRKSLKRRKLQELPKRRKLRKNSNLSHMMIKFFWMEMRYLCGSMTQFTLKHLSWD, (SEQ ID NO: 2849); XM_014118224.1, MARCKS, A_11, In, 88, high expression, KEALLLQEVLQAERLLLQEEQEGGGRGGGRGGGGRARRRPQGRGRRAGRAGAGAAAGAGAGAG AGAGAGGRGRGGRGGGRGGRRAPGG, (SEQ ID NO: 2850); XM_014106746.1, XRN1, A_12, Del, 8, essential, high expression, KGKWITYF, (SEQ ID NO: 2851); XM_014106745.1, XRN1, A_12, In, 17, essential, high expression, KGNGSLTFENPYIATFQ, (SEQ ID NO: 2852); XM_014106746.1, XRN1, A_12, In, 19, essential, high expression, KGNGSLTFENPYIATFQWL, (SEQ ID NO: 2853); XM 014111037.1, HSPE1, A_13, In, 10, essential, high expression, KPVPEGRAGV, (SEQ ID NO: 2854); XM 005638321.2, UBR1, A_13, Del, 10, high expression, KEHSGGGISK, (SEQ ID NO: 2855); XM 014113182.1, JAK1, A_9, In, 8, driver, high expression, SSRCYPSS, (SEQ ID NO: 2857); XM 005622315.2, CD55, A_9, Del, 8, high expression, NHVLILEI, (SEQ ID NO: 2858); XM 005619174.2, UIMC1, A_9, In, 8, high expression, RVWGTSVT, (SEQ ID NO: 2859); XM 014116983.1, TTF1, A_9, In, 8, essential, TSEERGFL, (SEQ ID NO: 2860); XM_005637736.2, TCF7L2, A_9, In, 8, driver, KVRSLHTR, (SEQ ID NO: 2861); XM_014112649.1, RBPJ, A_9, In, 9, essential, high expression, RTNGTGWLF, (SEQ ID NO: 2862); NM_001114605.1, ZC3H15, A_9, Del, 9, high expression, KKRKKMKFH, (SEQ ID NO: 2863); XM_005640445.2, PMS1, A_9, Del, 9, driver, KILKNINLT, (SEQ ID NO: 2864); XM_533327.6, DDX18, A_9, In, 10, essential, high expression, KEEEKEKNGG, (SEQ ID NO: 2865); XM_005623486.2, SYNE2, A_9, In, 10, high expression, KCGTKATKTF, (SEQ ID NO: 2866); XM_005628367.2, MKLN1, A_9, Del, 10, high expression, KPRLPWNIPC, (SEQ ID NO: 2867); XM_003432210.3, TAF1B, A_9, In, 10, essential, KAFKFSVQLD, (SEQ ID NO: 2868); XM_532047.5, CNTRL, A_9, In, 10, driver, NSTCTHKAGN, (SEQ ID NO: 2869); XM_536223.4, NOP14, A_9, Del, 11, essential, high expression, KASTTSMRMKN, (SEQ ID NO: 2870); XM_014113911.1, RERE, A_9, Del, 11, high expression, NLVMKGQTLGR, (SEQ ID NO: 2871); XM_014118133.1, ZNF292, A_9, Del, 11, high expression, KVLKKQKAPCK, (SEQ ID NO: 2872); XM_535048.6, ACADSB, A_9, Del, 11, high expression, KWESEPLPPVH, (SEQ ID NO: 2873); XM_005618622.2, RFC1, A_9, Del, 11, essential, KERVLIKKQSL, (SEQ ID NO: 2874); XM_005615403.2, RNMT, A_9, In, 11, essential, TRYHCFGPGMW, (SEQ ID NO: 2875); XM_014119810.1, KMT2C, A_9, In, 11, driver, KGARGQNYGSS, (SEQ ID NO: 2876); XM_005635475.2, PDS5B, A_9, In, 12, high expression, KCPCRTLQTSSY, (SEQ ID NO: 2877); XM_545992.5, WDR36, A_9, Del, 12, essential, KSSGKDFRMPCQ, (SEQ ID NO: 2878); XM_532047.5, CNTRL, A_9, In, 12, driver, NKCSTNSTIRTT, (SEQ ID NO: 2879); XM_014110821.1, DYNC1I2, A_9, Del, 13, essential, high expression, KEEKQKHCFKAWG, (SEQ ID NO: 2880); XM_005636978.2, KIF21A, A_9, In, 13, high expression, NARAFPYNQEKSI, (SEQ ID NO: 2881); XM_005623486.2, SYNE2, A_9, Del, 13, high expression, KMWNKSYKNFLIS, (SEQ ID NO: 2882); XM_535048.6, ACADSB, A_9, In, 13, high expression, NGNPSLFHLCINI, (SEQ ID NO: 2883); XM_005619174.2, UIMC1, A_9, Del, 13, high expression, KGLGNQCYLDLLL, (SEQ ID NO: 2884); XM_005623289.2, SCFD1, A_9, Del, 14, essential, high expression, KDLLISIQMLPLLF, (SEQ ID NO: 2885); XM_014111547.1, AP3B1, A_9, Del, 14, high expression, KQRKIEILPKMFHF, (SEQ ID NO: 2886); XM_005628367.2, MKLN1, A_9, In, 14, high expression, NQDCPGTSHVNRST, (SEQ ID NO: 2887); XM_014120966.1, ZNF236, A_9, In, 14, essential, NYKEEVTISAWFNP, (SEQ ID NO: 2888); XM_005638661.2, SCAPER, A_9, In, 15, high expression, SQKDKSPDELQSQGI, (SEQ ID NO: 2889); XM_014119579.1, UHRF1BP1L, A_9, Del, 15, high expression, KSQVIQCCLPVKSMI, (SEQ ID NO: 2890); XM_005617981.2, PRDM2, A_9, In, 16, high expression, SVAFIQERWTRVTCRQ, (SEQ ID NO: 2891); XM_003639290.3, SPAG9, A_9, In, 16, high expression, KVKHLAIFQPTFQLLK, (SEQ ID NO: 2892); XM_847826.3, SGOL1, A_9, Del, 16, essential, NWMNLSLPMMLTILIW, (SEQ ID NO: 2893); XM_005619222.2, SPDL1, A_9, In, 16, essential, NGGRCNCRSSMQTQRG, (SEQ ID NO: 2894); XM_003431923.4, HOXA11, A_9, Del, 16, driver, KLTETVYSTTQPTRSS, (SEQ ID NO: 2895); XM_005617468.2, BDP1, A_9, In, 19, essential, high expression, KCSFRSWTIRKCWRKNWKR, (SEQ ID NO: 2896); NM_001003057.1, TCOF1, A_9, In, 19, essential, RQGKKGKEEEGKKGLNQRP, (SEQ ID NO: 2897); XM_014110821.1, DYNC1I2, A_9, In, 20, essential, high expression, KKRSRSIASKHGADSRIPHC, (SEQ ID NO: 2898); XM_005633326.2, CEP57, A_9, In, 20, essential, NARVGSKTPGRTTGKETYAS, (SEQ ID NO: 2899); XM_005633512.2, POLD3, A_9, Del, 20, essential, KVTGGSEWSYLMMKQKKLKI, (SEQ ID NO: 2900); NM_001286956.1, HGS, A_9, Del, 20, essential, KSTTRIPMWPCTPWRSWSLW, (SEQ ID NO: 2901); XM_014112649.1, RBPJ, A_9, Del, 21, essential, high expression, KNKWNGMVVLNKSLNHVHLLE, (SEQ ID NO: 2902); XM_014113312.1, CHEK1, A_9, Del, 21, essential, KHTLTLGKKLIQLLLLCCIKS, (SEQ ID NO: 2903); XM_005640306.2, DYNC1I2, A_9, In, 22, essential, high expression, KKRSRSIASKHGADSRIPHCFF, (SEQ ID NO: 2904); XM_014106376.1, RBM26, A_9, In, 22, high expression, TGGTETSARCKEKETRNFRKAH, (SEQ ID NO: 2905); XM_532882.5, SMC6, A_9, In, 22, essential, NILVKRESKGLAGSRKLNQSRD, (SEQ ID NO: 2906); XM_545992.5, WDR36, A_9, In, 22, essential, SQAERTSECHVSEASTHHKVCS, (SEQ ID NO: 2907); XM_014112182.1, AHI1, A_9, Del, 23, high expression, KRKRRLNQFLIIMKTLMVMVFMK, (SEQ ID NO: 2908); XM_005637736.2, TCF7L2, A_9, Del, 23, driver, KSAFATYKVKAAASVHPLQMEAY, (SEQ ID NO: 2909); XM_005617468.2, BDP1, A_9, Del, 24, essential, high expression, KMLIQKLDHQEVLEKKLEEIILCL, (SEQ ID NO: 2910); XM_014106333.1, ERCC5, A_9, In, 25, driver, high expression, ITQAAAHTWLSQSSCCGCLPQTCGG, (SEQ ID NO: 2911); XM_005627570.2, IBTK, A_9, Del, 25, high expression, KGILKRKRFYQIFIIPHQMCLVSLI, (SEQ ID NO: 2912); XM_863127.3, ZFR, A_9, In, 26, essential, high expression, RSRIESFTKYQQQQQFYSWDSKSATL, (SEQ ID NO: 2914); XM_014108379.1, BCL2L13, A_9, In, 26, high expression, KSLQASDISGISGMYSGDHSSCQRLE, (SEQ ID NO: 2916); XM_535304.5, CHD9, A_9, Del, 26, high expression, KIQLLYLVNSLYNYLWRIQVKKMLQL, (SEQ ID NO: 2917); XM_005634176.2, GINS1, A_9, Del, 26, essential, KIASISYPDGSVSSWLDREFWSTCCP, (SEQ ID NO: 2918); XM_014119810.1, KMT2C, A_9, Del, 26, driver, KRSKRTKLWFLLINILHGKNLWVLVR, (SEQ ID NO: 2919); XM_014110679.1, SP3, A_9, In, 27, high expression, KSPDRSGRVRRRFPHPLPPPPPSLLTM, (SEQ ID NO: 2920); XM_005615403.2, RNMT, A_9, Del, 27, essential, NAISLFWTWDVVKVEICSNGKREELTS, (SEQ ID NO: 2921); XM_003639290.3, SPAG9, A_9, Del, 28, high expression, KGQAFGNFSADFSAPQVIQLRSLNHLLI, (SEQ ID NO: 2922); XM_851972.4, ARV1, A_9, In, 28, high expression, TQLHFAAESIIIIQLWKALAHSSCHLGA, (SEQ ID NO: 2923); XM_014112201.1, CAST, A_9, In, 28, high expression, NPRSKAKGTQRAKKPTQACIRYGKQACS, (SEQ ID NO: 2924); XM_003432210.3, TAF1B, A_9, Del, 28, essential, KGLQVFSSTGLKETLQEPVSTDMASRGF, (SEQ ID NO: 2925); XM_005629758.2, PSD3, A_9, Del, 29, high expression, NLHQKVLMRRLMEHIQRPSVVSGALPIHS, (SEQ ID NO: 2926); XM_005633326.2, CEP57, A_9, Del, 29, essential, KCKSWKQNSGKNNRKGNVCKLRQPSCRLG, (SEQ ID NO: 2927); XM_532252.6, SEC63, A_9, In, 30, essential, high expression, KEAFKKKTYTCALTTVKATETKAGKWNCWE, (SEQ ID NO: 2928); XM_850840.5, VPS13B, A_9, Del, 32, high expression, KWIHLMEAWLKPHLGTVVPRIVELAVTVLKSE, (SEQ ID NO: 2929); XM_005630347.2, CEBPZ, A_9, Del, 34, essential, high expression, KRKEISVTPVYLYLLKSLATYWMKIWDPSLITLA, (SEQ ID NO: 2930); XM_014110819.1, DYNC1I2, A_9, In, 35, essential, high expression, KKRSRSIASKHGADSRIPHCPSSYVSILQICEHTK, (SEQ ID NO: 2931); XM_533539.4, KIAA0020, A_9, Del, 36, high expression, KDFIKTVILVRQRHSQGKLSRKVDLKSHLRTLRNVP, (SEQ ID NO: 2932); XM_005637010.2, FAM60A, A_9, In, 37, high expression, KLESCGRCKGRTQSEDYFETKESENSIWKQDKKQPDQ, (SEQ ID NO: 2933); XM_005635476.2, PDS5B, A_9, Del, 39, high expression, NLDLQHQKKRKKKKDKVETQNRSQRANSTEHQGEHNRAV, (SEQ ID NO: 2934); NM_001003057.1, TCOF1, A_9, Del, 39, essential, KTRKKRKRRRRQKRPQPKTLTHYSRRKRRKRRRQQSKLS, (SEQ ID NO: 2935); NM_001286956.1, HGS, A_9, In, 40, essential, SQRQESPCGLVRPGGHGVCGEELWPDGARRGGQQADHGGA, (SEQ ID NO: 2936); XM_005635475.2, PDS5B, A_9, In, 41, high expression, IWTFSTRRRGRRRKTKWKHRTEVKEQTAPNIKESTTESRIS, (SEQ ID NO: 2937); XM_005635474.2, PDS5B, A_9, In, 42, high expression, IWTFSTRRRGRRRKTKWKHRTEVKEQTAPNIKESTTEQSRIS, (SEQ ID NO: 2938); XM_014110679.1, SP3, A_9, Del, 44, high expression, KIPGPLRPCPPPLPASSPAAAAFAPHHVTLSRHRSPCWPLPAAR, (SEQ ID NO: 2939); XM_005635476.2, PDS5B, A_9, In, 47, high expression, IWTFSTRRRGRRRKTKWKHRTEVKEQTAPNIKESTTEPCRTLQTSSY, (SEQ ID NO: 2940); XM_535332.5, ZCCHC17, A_9, Del, 48, high expression, NIETGSHLTLTAQTLRVTQARGQGTHQKTARQQRRRRRRRSIRRNTRS, (SEQ ID NO: 2941); XM_014116983.1, TTF1, A_9, Del, 48, essential, NIGGKRLPVMLSRRAHLLGSPCPSRVRIRRSWKACFLWAQRAESHNYQ, (SEQ ID NO: 2942); XM_863127.3, ZFR, A_9, In, 49, essential, high expression, SKSRFASRSKAQYSSQKDSRRENEEADAERRVLAKTRRRRTLENGNEAL, (SEQ ID NO: 2943); XM_005634067.2, ERCC5, A_9, Del, 54, driver, high expression, NYASCSSHLAFPIQLLRMLTSNLWWMIPGDRFCGGNQIWTKLETLFWLEQDEDR, (SEQ ID NO: 2944); XM_533327.6, DDX18, A_9, Del, 60, essential, high expression, KRRREREKWWMMLGLIPKNQKLKTKGTLKKGPRLLKRQKTMGRSQMMRKRTVKCPAYPWD, (SEQ ID NO: 2945); XM_014113182.1, JAK1, A_9, Del, 60, driver, high expression, KFQMLPLFLMPVLWSISLLRDSMIWSNAWLPFETPRLSRMGTILRMSVWEWLSWPSPTMP, (SEQ ID NO: 2946); XM_014106333.1, ERCC5, A_9, Del, 64, driver, high expression, NYASCSSHLAFPIQLLRMLTSNLWWMIPGDRFCGGNQIWTKLENFVSVILAGTGRRQMSLCFQY, (SEQ ID NO: 2947); XM_005617981.2, PRDM2, A_9, Del, 73, high expression, KCRLHPRKVDTRHLPAVTKTAAATGAGLQTRRLRCRACRLLWARPERAAPALRQARCRPRPSGPS RMSNLQLR, (SEQ ID NO: 2948); XM_005635475.2, PDS5B, A_9, Del, 75, high expression, NLDLQHQKKRKKKKDKVETQNRSQRANSTEHQGEHNREQNLLKLVQLNPHNPHHRKDEEDHQK RHHHHNQKKMSV, (SEQ ID NO: 2949); XM_005635474.2, PDS5B, A_9, Del, 76, high expression, NLDLQHQKKRKKKKDKVETQNRSQRANSTEHQGEHNRAEQNLLKLVQLNPHNPHHRKDEEDHQ KRHHHHNQKKMSV, (SEQ ID NO: 2950); XM_014121429.1, SPATA5, U_10, In, 13, essential, FKRQLPWKNSLYL, (SEQ ID NO: 2951); XM_014121429.1, SPATA5, U_10, Del, 13, essential, FQKAIALEKFTLL, (SEQ ID NO: 2952); XM_014120710.1, GOLPH3L, U_10, Del, 15, high expression, FPKVRLGTPSNYSTS, (SEQ ID NO: 2953); XM_014121460.1, SLC35F5, U_10, Del, 22, high expression, FALCGFWQICRIKKHFQTHRLL, (SEQ ID NO: 2954); XM_014121460.1, SLC35F5, U_10, In, 23, high expression, LLCVVSGKFVVSRSTFRHTGCYS, (SEQ ID NO: 2955); XM_005637886.1, NSMCE4A, U_13, In, 19, essential, LHRKPGGYTECQRQINRGP, (SEQ ID NO: 2957); XM_005624991.1, MED13, U_16, Del, 13, high expression, FFPCPPFPTLLQP, (SEQ ID NO: 2958); XM_005624991.1, MED13, U_16, In, 31, high expression, SFLVPRFRLFFNRSLVFNLCSLSGRILPFPG, (SEQ ID NO: 2959); XM_005630622.2, CTSK, U_22, In, 8, high expression, LLPSKTVM, (SEQ ID NO: 2960); XM_005630622.2, CTSK, U_22, Del, 13, high expression, FTALKDSHVKLNT, (SEQ ID NO: 2961); XM_005618433.2, MORF4L1, U_25, Del, 21, high expression, LLLQVSEYCAFMGLFFMKQSV, (SEQ ID NO: 2962); XM_014111218.1, ZC3H11A, U_9, In, 8, high expression, FLFYMYQR, (SEQ ID NO: 2963); XM_540560.5, ELP4, U_9, In, 8, essential, SRKIIPNS, (SEQ ID NO: 2964); XM_014109749.1, ITSN1, U_9, Del, 9, high expression, FNLGYLNLF, (SEQ ID NO: 2965); XM_540560.5, ELP4, U_9, Del, 10, essential, FQKNYPQLLI, (SEQ ID NO: 2966); XM_536488.5, C6, U_9, In, 11, high expression, FEKRSGTQVYH, (SEQ ID NO: 2967); XM_014118812.1, CLOCK, U_9, In, 13, high expression, FSNHDRWKHNICV, (SEQ ID NO: 2968); XM_005627395.2, XPOS, U_9, In, 13, essential, FGKCDQPDVSDIR, (SEQ ID NO: 2969); XM_005618779.2, RYR2, U_9, Del, 14, high expression, LLLISLTLLWGSKH, (SEQ ID NO: 2970); XM_532552.5, UTP11L, U_9, Del, 15, essential, high expression, LTPKRKLSSLILPLT, (SEQ ID NO: 2971); XM_014109749.1, ITSN1, U_9, In, 16, high expression, SIWVTSTCFSTDMGAS, (SEQ ID NO: 2972); XM_003435295.1, SLFN5, U_9, Del, 16, high expression, FLEAGLWTLVYQRSRE, (SEQ ID NO: 2973); XM_014106522.1, FOXL2, U_9, In, 17, driver, FQSPLEKTLQYQQNTTT, (SEQ ID NO: 2974); XM_003435295.1, SLFN5, U_9, In, 18, high expression, FSKLGCGHWSIREAGSNL, (SEQ ID NO: 2975); XM_014106522.1, FOXL2, U_9, Del, 19, driver, FSKSTRKNTTISAKYNNMN, (SEQ ID NO: 2976); XM_014111218.1, ZC3H11A, U_9, Del, 20, high expression, FSILHVPKVTAVPSVTVKLH, (SEQ ID NO: 2977); XM_005635440.2, EXOSC8, U_9, Del, 21, essential, FSSRFACLKGLWNLWSITGDF, (SEQ ID NO: 2978); XM_005635440.2, EXOSC8, U_9, In, 27, essential, FLPASRVSKDCGTSGVLQEIFERELPS, (SEQ ID NO: 2979); XM_536488.5, C6, U_9, Del, 32, high expression, FRKKKWNTGVPLTRCPRNTKVPFCREQRNPYP, (SEQ ID NO: 2980); XM_014116728.1, INTS2, U_9, Del, 32, essential, LSHLSYLRVQSIWRKLQMYFVFYKQSSLPCFL, (SEQ ID NO: 2981); XM_014114900.1, SRSF11, U_9, Del, 35, essential, FMLLVYFVVIHLCQSHPVSALLSSMIRTQQLWHSI, (SEQ ID NO: 2982); XM_005623772.2, SERPINA3, U_9, Del, 41, high expression, FPERSAHWSLFVSTDPFCSAYTTKIRRTSSSGARSPTPIKP, (SEQ ID NO: 2983); XM_005623772.2, SERPINA3, U_9, In, 41, high expression, FRKDRPTGHCSFQQTLSVLHTPQRYGEHPLLGQGHQPQSSL, (SEQ ID NO: 2984); XM_005621019.2, ACTB, C_10, In, 10, essential, high expression, HRRVPLVATP, (SEQ ID NO: 2986); XM_005637736.2, TCF7L2, C_10, Del, 17, driver, PQTPSHRLSRPSRCRCP, (SEQ ID NO: 2987); XM_014114858.1, TRIM28, C_10, In, 18, essential, PEPAWCWPECPGAIQWSW, (SEQ ID NO: 2988); XM_005621019.2, ACTB, C_10, Del, 36, essential, high expression, PPQSPPRGHPLKLSPASVSSQAWNPAASMKLPSTPS, (SEQ ID NO: 2989); XM_014108583.1, ARID2, C_11, In, 12, driver, PEPADVAVRNSF, (SEQ ID NO: 2990); XM_014120240.1, MTA3, C_11, In, 14, high expression, RSAPRASCSTGSRS, (SEQ ID NO: 2991); XM_543674.5, SMARCD1, C_11, Del, 26, driver, essential, high expression, PRLKGCTAPRCPERPIRDQACCQAAE, (SEQ ID NO: 2992); XM_014108583.1, ARID2, C_11, Del, 37, driver, PGTRRRRCPEQFLRRISGEKLLKSSTFPEVVLTLPLL, (SEQ ID NO: 2993); XM_014120240.1, MTA3, C_11, Del, 83, high expression, PLRPPSQLQHRLPELKRSPLGLLRANCPALRSKPREGVPGAGIILNYYQQFTRHPTPAAQPAPCPPEA KQIMSTLRIPPVTRT, (SEQ ID NO: 2994); XM_543674.5, SMARCD1, C_11, In, 86, driver, essential, high expression, PASRAVPLPDARSGLSETRHAARQPNDTSGTFHGTPWLWGEPFSPTWPGPVRDGPVPQETCTSADT AGPAAGGPKSKPQCKEKEDG, (SEQ ID NO: 2995); XM_005627791.2, TSPYL1, C_11, Del, 98, high expression, PLAKAWQRRRCRCRWQRTAAWKMGVGAPGRAGRVRGALRGRRTCGRRRRPRSCRPATASLRRR RRRRRRRSPGWRQTRREGKSWPGGRVPGRPRGPDA, (SEQ ID NO: 2996); XM_014106524.1, RBP1, C_13, In, 10, high expression, QELASGAPLP, (SEQ ID NO: 2997); XM_014111383.1, RBM10, C_13, In, 38, driver, essential, PMAPGRRQSRRPGWVSSRAPSFSLTPTSAPFIPSLPQH, (SEQ ID NO: 2998); XM_014106524.1, RBP1, C_13, Del, 44, high expression, PGAGFWGPTALGTVLWIFLPTLCPSVPRHPLQMLMWPCAKLPTC, (SEQ ID NO: 2999); XM_014115020.1, INTS3, C_14, In, 16, essential, high expression, PAMPPWNGHSRCQSVY, (SEQ ID NO: 3001); XM_005637886.1, NSMCE4A, C_14, In, 16, essential, RPAGDPGTRAWPRCCR, (SEQ ID NO: 3002); XM_005637886.1, NSMCE4A, C_14, Del, 48, essential, PPRRGPGDPGLAALLQVITPARTCSRVMSTFPQTLFFVCLVFCFFFFA, (SEQ ID NO: 3003); XM_014115020.1, INTS3, C_14, Del, 56, essential, high expression, PRHAALEWPQSLSECLLSPVWYAKAPHSMRKSAWASSPSSSLNLPKLKSVTGTWLW, (SEQ ID NO: 3004); XM_005640152.1, IRF4, C_15, In, 46, driver, PDTRSRGPSPASPAAPGPRQQRPLAPTRVAPRCPPSSAPGSAPAGF, (SEQ ID NO: 3005); XM_005640152.1, IRF4, C_15, Del, 64, driver, PRHALPRPEPGLPRGAGSPPAAPPRPDPGRAALPAQLRPRLRPSGLLSFISSSFPPRHLLGFTS, (SEQ ID NO: 3006); XM_014116368.1, GOSR2, C_16, Del, 23, essential, PAFILIWFILKTIVMECLVWAGP, (SEQ ID NO: 3007); XM_005640418.2, AAMP, C_9, In, 8, essential, high expression, PGDPELPW, (SEQ ID NO: 3008); XM_014109694.1, DONSON, C_9, In, 8, essential, RPRPRPRF, (SEQ ID NO: 3009); XM_014111168.1, SPEN, C_9, In, 10, driver, essential, high expression, PITSCTRRTL, (SEQ ID NO: 3010); XM_014107007.1, DIDO1, C_9, In, 15, essential, high expression, PWPLRGPKGAASQSV, (SEQ ID NO: 3011); XM_014109694.1, DONSON, C_9, Del, 18, essential, PPPPPPPILMVKPDAKLT, (SEQ ID NO: 3012); XM_536612.5, PELP1, C_9, Del, 35, essential, high expression, PHLLPLCRALWPCRRHSWYPRRLLVEEDPLPWRKT, (SEQ ID NO: 3013); XM_536612.5, PELP1, C_9, In, 38, essential, high expression, PTSSPCAGPCGPAAATVGTRGDSWWRRTPCPGGRPDGY, (SEQ ID NO: 3014); XM_014107007.1, DIDO1, C_9, Del, 42, essential, high expression, PLATSWAQGGRIPVSLKDPEAKHQISCQDPGGCSLSSSRSRG, (SEQ ID NO: 3015); XM_005634093.2, F7, C_9, In, 42, high expression, PSLPHPGGSPGCPAPAKARQLVPGGAAGRLPGERVPGGAVLL, (SEQ ID NO: 3016); XM_547535.6, SMG5, C_9, In, 42, essential, PRGEQRARGEGPPYQAALPGCGGGCASTRPHPLQQNCLSRSV, (SEQ ID NO: 3017); XM_014112276.1, COL4A3BP, C_9, In, 43, high expression, RAGAGTPPFPAPRALTPAPGRFLRRGSGSGGWSPARLSRLHVG, (SEQ ID NO: 3018); XM_547535.6, SMG5, C_9, Del, 49, essential, PQGRAASPRRRSSIPSGFTGLWWRLCIDSTSSFATKLLIKKCLNQRTLA, (SEQ ID NO: 3019); XM_532250.5, QRSL1, G_10, In, 9, essential, EGDRTLSTP, (SEQ ID NO: 3022); XM_014120239.1, SOS1, G_11, Del, 42, essential, high expression, GVRKDLREKGAQERGKELVPRSRDKFIRLLSLVTMLFNMLKN, (SEQ ID NO: 3024); XM_005635998.1, DTYMK, G_11, Del, 81, essential, GLLSGAPAAQTAGAASATRAAAASGPGEAPAEAASSGSRGFGSRPRPRTKGRPSAPRAAPLCSGPA RPGPRLRSALCFLGR, (SEQ ID NO: 3026); XM_005619450.1, PWWP2A, G_14, Del, 9, driver, GTIRQTRTS, (SEQ ID NO: 3028); XM_014110360.1, PLD1, G_15, Del, 36, high expression, GAMHLLCREVAPGHCLPFQALPTSTTAACWEGATAK, (SEQ ID NO: 3030); XM_014110360.1, PLD1, G_15, In, 77, high expression, GRCTCSAERLLQVTVSRSRLCQHQRPQHAGKARQRNECACAGHGDGPFGHGRTGVPRWPLRPRPP AAVLQGCPWLSF, (SEQ ID NO: 3031); XM_014118660.1, RECQL4, G_9, Del, 21, driver, GARTPAVSPRRCRSPAAGGRT, (SEQ ID NO: 3032); XM_005642470.2, MED4, G_9, Del, 30, essential, high expression, GAARARRAGTARGSGCCLRWRTWRSCRENL, (SEQ ID NO: 3033); XM_014117894.1, ABCA1, G_9, In, 32, high expression, GPQDQVPQLVRGQQLQSPLWWQRHGRRCWQLL, (SEQ ID NO: 3035); XM_536395.5, VCL, G_9, In, 36, essential, high expression, GFSPGGVQNQILLSGGNRRITFVCFCLYLAPEQRLC, (SEQ ID NO: 3036); XM_014117894.1, ABCA1, G_9, Del, 43, high expression, GASRSSPSTGTRTTTTKPSLVATARTKMLAASMTILRLLIAMI, (SEQ ID NO: 3037); XM_014114365.1, SETD1A, G_9, Del, 48, essential, GPALREKKLGPPPAQLHLPAPAPRPQRPPTRACPSLNIAAWIPASRCC, (SEQ ID NO: 3039); XM_014118660.1, RECQL4, G_9, In, 49, driver, GLGRPPCLPADAGAQLLGAALESGSDAEASRRRLCARLCGGRGRLRGEA, (SEQ ID NO: 3040); XM_005631747.1, SART1, G_9, In, 94, essential, AGSGGKYHPGGVKKTPWGERGGRDDGGGQHRRRHRAAAAAPGAQKTQAPERRWREQRWRTSE AESGAWGRARGRAARDRSRDTQRRARAGARPG, (SEQ ID NO: 3041); XM_547275.5, DNTTIP2, A_8, In, 8, essential, high expression, TNGAKTPF, (SEQ ID NO: 3042); XM_014112366.1, MPHOSPH10, A_8, Del, 8, essential, high expression, NLGSFKGK, (SEQ ID NO: 3043); XM_003639706.3, RIF1, A_8, In, 8, essential, high expression, KRNGFCKI, (SEQ ID NO: 3044); XM_014121226.1, EIF3M, A_8, In, 8, essential, high expression, THPFKAPL, (SEQ ID NO: 3045); XM_005623158.1, TRIP11, A_8, Del, 8, driver, high expression, KLKNLRIK, (SEQ ID NO: 3046); XM_005625956.2, APPL2, A_8, In, 8, high expression, KGKREGKD, (SEQ ID NO: 3047); NM_001168012.1, CCDC66, A_8, In, 8, high expression, SKKADGTA, (SEQ ID NO: 3048); XM_005618961.2, DDX50, A_8, In, 8, high expression, IKASVILR, (SEQ ID NO: 3049); XM_005618927.2, JMJD1C, A_8, Del, 8, high expression, KVQTQLHV, (SEQ ID NO: 3050); XM_014119919.1, NUB1, A_8, In, 8, high expression, IKLGSEML, (SEQ ID NO: 3051); XM_014120122.1, SORBS2, A_8, In, 8, high expression, KFDPQKTD, (SEQ ID NO: 3052); XM_005628995.2, ZCCHC11, A_8, Del, 8, high expression, KISKMIFV, (SEQ ID NO: 3053); XM_014118133.1, ZNF292, A_8, Del, 8, high expression, KKKMKWMS, (SEQ ID NO: 3054); XM_546411.5, SRPR, A_8, In, 8, high expression, KGGQEGEL, (SEQ ID NO: 3055); XM_014110029.1, MRPL1, A_8, In, 8, high expression, KYERQSIR, (SEQ ID NO: 3056); XM_014116993.1, NUP214, A_8, In, 8, driver, essential, SHSLPTIL, (SEQ ID NO: 3057); XM_014107179.1, AURKA, A_8, In, 8, essential, KAMGFGRF, (SEQ ID NO: 3058); XM_543181.4, CENPJ, A_8, In, 8, essential, NWWFFRSG, (SEQ ID NO: 3059); XM_546202.5, DCP2, A_8, In, 8, essential, TSSTKTSG, (SEQ ID NO: 3060); XM_014118166.1, MMS22L, A_8, Del, 8, essential, KFPVQGCN, (SEQ ID NO: 3061); XM_545510.4, SPC25, A_8, In, 8, essential, RELVKVGC, (SEQ ID NO: 3062); XM_014121533.1, TAMM41, A_8, Del, 8, essential, KIGTITLS, (SEQ ID NO: 3063); XM_005615312.2, ZNF407, A_8, In, 8, essential, ICSGNGFP, (SEQ ID NO: 3064); XM_014117483.1, LIPT1, A_8, Del, 8, essential, KSMIECRI, (SEQ ID NO: 3065); NM_001003306.2, ATP1A1, A_8, In, 9, driver, essential, high expression, KGQERKGYG, (SEQ ID NO: 3066); XM_535848.5, SMC4, A_8, In, 9, essential, high expression, KAEITRRKC, (SEQ ID NO: 3067); XM_546107.5, TFAM, A_8, In, 9, essential, high expression, KRVNNAWKT, (SEQ ID NO: 3068); XM_005636122.2, SBNO1, A_8, Del, 9, essential, high expression, KMEDMIWEF, (SEQ ID NO: 3069); XM_535952.5, SSB, A_8, Del, 9, essential, high expression, KMKKESKIK, (SEQ ID NO: 3070); XM_531814.5, MSH6, A_8, Del, 9, driver, high expression, KRLLIFFLL, (SEQ ID NO: 3071); XM_844219.5, RPL22, A_8, In, 9, driver, high expression, KEASSEVYP, (SEQ ID NO: 3072); XM_005623082.2, ANKRD12, A_8, In, 9, high expression, IKVGKKHKR, (SEQ ID NO: 3073); XM_014115338.1, ASH1L, A_8, In, 9, high expression, KERKKSSVD, (SEQ ID NO: 3074); NM_001003343.1, DMD, A_8, Del, 9, high expression, KWKTAMDLI, (SEQ ID NO: 3075); XM_014119556.1, EEA1, A_8, Del, 9, high expression, NSLNEKLKI, (SEQ ID NO: 3076); XM_005615362.2, LMAN1, A_8, In, 9, high expression, KGGIPEGPS, (SEQ ID NO: 3077); XM_014118865.1, UTRN, A_8, Del, 9, high expression, NPRVNGKKS, (SEQ ID NO: 3078); NM_001012395.1, UTRN, A_8, Del, 9, high expression, NPRVNVKKS, (SEQ ID NO: 3079); XM_005628995.2, ZCCHC11, A_8, In, 9, high expression, KCIIYRCGI, (SEQ ID NO: 3080); XM_005623314.2, ARHGAPS, A_8, In, 9, high expression, KYDRKFLFI, (SEQ ID NO: 3081); XM_014112733.1, ARID4B, A_8, In, 9, high expression, KGKRQCPTL, (SEQ ID NO: 3082); XM_014112733.1, ARID4B, A_8, Del, 9, high expression, KREKAVPHT, (SEQ ID NO: 3083); XM_014117434.1, IL1R1, A_8, In, 9, high expression, KKEYNHHHA, (SEQ ID NO: 3084); XM_005622047.1, NEXN, A_8, Del, 9, high expression, KVRPHLLIK, (SEQ ID NO: 3085); XM_014116764.1, ATAD5, A_8, Del, 9, essential, NVLPLFLTV, (SEQ ID NO: 3086); XM_844090.4, INO80, A_8, In, 9, essential, KEIQGGKET, (SEQ ID NO: 3087); XM_534964.5, KIF11, A_8, Del, 9, essential, KSIHRNFAS, (SEQ ID NO: 3088); XM_005641214.2, POLA1, A_8, Del, 9, essential, NQREGAKKL, (SEQ ID NO: 3089); XM_848505.4, TWISTNB, A_8, In, 9, essential, KEEERPRVL, (SEQ ID NO: 3090); XM_005623419.2, WDHD1, A_8, Del, 9, essential, KKRKISEKS, (SEQ ID NO: 3091); XM_005637332.2, KDM5A, A_8, Del, 9, driver, KGLAGRLKK, (SEQ ID NO: 3092); XM_014115024.1, SETBP1, A_8, Del, 9, driver, KYIRERMSV, (SEQ ID NO: 3093); XM_014115260.1, AHCTF1, A_8, Del, 10, essential, high expression, KRLLKELKNP, (SEQ ID NO: 3094); XM_547275.5, DNTTIP2, A_8, Del, 10, essential, high expression, NEWCKNAVLI, (SEQ ID NO: 3095); XM_005635844.2, PSMD1, A_8, Del, 10, essential, high expression, NQLTRDWKAL, (SEQ ID NO: 3096); XM_005630143.2, NBAS, A_8, Del, 10, essential, high expression, KSKIESPSTH, (SEQ ID NO: 3097); XM_014106985.1, ZMYND8, A_8, In, 10, essential, high expression, KAQTGQPSGD, (SEQ ID NO: 3098); XM_014106985.1, ZMYND8, A_8, In, 10, essential, high expression, KARHQGNRKQ, (SEQ ID NO: 3099); NM_001003343.1, DMD, A_8, In, 10, high expression, NGKQQWILSK, (SEQ ID NO: 3100); XM_543139.5, PDS5B, A_8, Del, 10, high expression, KVTRETTLIL, (SEQ ID NO: 3101); XM_005623023.2, ROCK1, A_8, In, 10, high expression, MGNTFGKEDP, (SEQ ID NO: 3102); XM_535467.5, SLC12A1, A_8, In, 10, high expression, KLEESSLDRD, (SEQ ID NO: 3103); XM_005635161.2, STAU1, A_8, In, 10, high expression, NRGWKKSNLF, (SEQ ID NO: 3104); XM_005626151.2, UGP2, A_8, Del, 10, high expression, KYYRSTIIVV, (SEQ ID NO: 3105); XM_014119900.1, WDR60, A_8, In, 10, high expression, KHQAGLCSVQ, (SEQ ID NO: 3106); XM_546524.5, DLAT, A_8, Del, 10, high expression, KKGKKSMRVN, (SEQ ID NO: 3107); XM_532929.5, FAM98A, A_8, In, 10, high expression, IKGDISKSST, (SEQ ID NO: 3108); XM_540963.4, KIAA1109, A_8, Del, 10, high expression, KVLITQMMKH, (SEQ ID NO: 3109); XM_542274.5, TMEM126B, A_8, Del, 10, high expression, NLNILEEKRL, (SEQ ID NO: 3110); XM_535814.5, TTC14, A_8, In, 10, high expression, KFTSKFTQYI, (SEQ ID NO: 3111); XM_005617578.1, HEATR3, A_8, Del, 10, essential, KIRTEILLRT, (SEQ ID NO: 3112); XM_014118166.1, MMS22L, A_8, In, 10, essential, NFQCRAATDV, (SEQ ID NO: 3113); XM_536500.4, NUP155, A_8, In, 10, essential, RSSGSWASFL, (SEQ ID NO: 3114); XM_534728.4, THOC5, A_8, In, 10, essential, NSKSSQSVSV, (SEQ ID NO: 3115); XM_014116982.1, TTF1, A_8, In, 10, essential, KEKNFTPPGT, (SEQ ID NO: 3116); XM_534660.5, VPS33A, A_8, Del, 10, essential, NVSQEKTESL, (SEQ ID NO: 3117); XM_014110313.1, TRIO, A_8, In, 10, essential, KGLLDARIPV, (SEQ ID NO: 3118); XM_014113891.1, CAMTA1, A_8, Del, 10, driver, NFSRAGVLLC, (SEQ ID NO: 3119); XM_847012.4, PDCD1LG2, A_8, Del, 10, driver, KTQQKTLSPP, (SEQ ID NO: 3120); XM_005641516.2, ATRX, A_8, Del, 11, driver, essential, high expression, KQRNLQVRDAF, (SEQ ID NO: 3121); NM_001003306.2, ATP1A1, A_8, Del, 11, driver, essential, high expression, KRPRKKGIWMN, (SEQ ID NO: 3122); XM_014111157.1, COPA, A_8, Del, 11, essential, high expression, KTCPLVQWSPT, (SEQ ID NO: 3123); XM_005626022.2, EIF5B, A_8, In, 11, essential, high expression, KERTKRQKTKF, (SEQ ID NO: 3124); XM_005619358.2, ZNF131, A_8, In, 11, essential, high expression, KKDCRNFKCYH, (SEQ ID NO: 3125); XM_005630347.2, CEBPZ, A_8, Del, 11, essential, high expression, KVLRKTQKMKI, (SEQ ID NO: 3126); XM_014122254.1, ALAS1, A_8, Del, 11, high expression, KMTIPIEFLKL, (SEQ ID NO: 3127); XM_005625956.2, APPL2, A_8, Del, 11, high expression, KRKTRRQRLRL, (SEQ ID NO: 3128); XM_536025.4, BZW1, A_8, Del, 11, high expression, KTTSQNPLSLE, (SEQ ID NO: 3129); XM_014110832.1, EIF4G3, A_8, In, 11, high expression, NKNCGRKWRRS, (SEQ ID NO: 3130); XM_005618927.2, JMJD1C, A_8, In, 11, high expression, RCKLSYMSKIL, (SEQ ID NO: 3131); XM_014106956.1, ADNP, A_8, Del, 11, high expression, KRLPCKVTESS, (SEQ ID NO: 3132); XM_845089.4, CXCL13, A_8, Del, 11, high expression, KMLLQLNQLQC, (SEQ ID NO: 3133); XM_005616083.2, PEG3, A_8, Del, 11, high expression, KTSSIGTMRPL, (SEQ ID NO: 3134); XM_014114998.1, TOR1AIP1, A_8, Del, 11, high expression, KLRNGLNQPQE, (SEQ ID NO: 3135); XM_545208.5, PIK3CA, A_8, Del, 11, driver, essential, KLEVCCYHLNN, (SEQ ID NO: 3136); XM_535194.5, KIN, A_8, In, 11, essential, KEICPRRNHGD, (SEQ ID NO: 3137); XM_535150.5, MTPAP, A_8, In, 11, essential, ISEIFIPTWTY, (SEQ ID NO: 3138); XM_532882.5, SMC6, A_8, Del, 11, essential, NTKIFKINWKR, (SEQ ID NO: 3139); XM_547651.5, THOC1, A_8, Del, 11, essential, NVPLTKLSEVF, (SEQ ID NO: 3140); XM_014107997.1, KNTC1, A_8, In, 11, essential, TEAQAPEVNRS, (SEQ ID NO: 3141); XM_005626020.2, REV1, A_8, In, 11, essential, NENRQVYTCCN, (SEQ ID NO: 3142); XM_535077.4, TGS1, A_8, In, 11, essential, KQEQKGNWSAS, (SEQ ID NO: 3143); XM_014120072.1, WRN, A_8, In, 11, driver, IGKNSTAAKTS, (SEQ ID NO: 3144); XM_014118114.1, SENP6, A_8, Del, 12, essential, high expression, KSIRTLIQQKHL, (SEQ ID NO: 3145); XM_014110834.1, CWC22, A_8, In, 12, essential, high expression, KERREKKFFFRK, (SEQ ID NO: 3146); XM_014115839.1, BAZ1A, A_8, Del, 12, high expression, KQDRIFRVFQNH, (SEQ ID NO: 3147); XM_847165.4, CCDC88A, A_8, Del, 12, high expression, KHWMVLKTLLFS, (SEQ ID NO: 3148); XM_857314.4, PHF20L1, A_8, In, 12, high expression, SEIGRKKLNSIW, (SEQ ID NO: 3149); XM_005635835.2, SP100, A_8, In, 12, high expression, KQSSKSEKERQT, (SEQ ID NO: 3150); XM_005638476.2, VPS13C, A_8, Del, 12, high expression, KLVCDALSSVTL, (SEQ ID NO: 3151); XM_005619248.2, CLINT1, A_8, Del, 12, high expression, KIGEEFTSHCCF, (SEQ ID NO: 3152); XM_005630332.2, FEZ2, A_8, Del, 12, high expression, KMDHHLLKIFKY, (SEQ ID NO: 3153); XM_014117434.1, IL1R1, A_8, Del, 12, high expression, KERVQSSPCLIF, (SEQ ID NO: 3154); XM_005633985.2, MYCBP2, A_8, In, 12, high expression, ILVRICSGFFII, (SEQ ID NO: 3155); XM_014117917.1, ZNF483, A_8, Del, 12, high expression, KSPLCVKNVGKF, (SEQ ID NO: 3156); XM_014111981.1, HBS1L, A_8, Del, 12, essential, KRNLTDLKVKRN, (SEQ ID NO: 3157); XM_532052.5, MED23, A_8, Del, 12, essential, KMWRRNIGSGSQ, (SEQ ID NO: 3158); XM_542577.3, NUFIP1, A_8, In, 12, essential, KEKKGTSFSLFL, (SEQ ID NO: 3159); NM_001003264.1, SRP72, A_8, Del, 13, essential, high expression, KRRENYLKIMTQR, (SEQ ID NO: 3160); XM_014111218.1, ZC3H11A, A_8, In, 13, high expression, NRYKRREETSRRQ, (SEQ ID NO: 3161); XM_014108520.1, ZCRB1, A_8, Del, 13, high expression, KRKFLSQKKKLKK, (SEQ ID NO: 3162); XM_014118258.1, OGFRL1, A_8, In, 13, high expression, SSTRRTWRGNRQS, (SEQ ID NO: 3163); XM_014117181.1, R3HDM2, A_8, In, 13, high expression, TGGRICKQKQVYF, (SEQ ID NO: 3164); XM_544672.5, SECISBP2L, A_8, In, 13, high expression, TTGSFIKGSWKKE, (SEQ ID NO: 3165); NM_001003154.2, TRDN, A_8, Del, 13, high expression, KSQGKLLKPNKGL, (SEQ ID NO: 3166); XM_534048.5, DENNDSA, A_8, In, 13, high expression, ITFQTPEAASLRP, (SEQ ID NO: 3167); XM_547249.5, STXBP3, A_8, Del, 13, high expression, NLKTTTKLMKRAK, (SEQ ID NO: 3168); XM_535814.5, TTC14, A_8, Del, 13, high expression, KIYLKIYSIYLIR, (SEQ ID NO: 3169); XM_014116764.1, ATAD5, A_8, In, 13, essential, MFCPCFSLFKFSH, (SEQ ID NO: 3170); XM_844090.4, INO80, A_8, Del, 13, essential, KRNSRRKRNLKPS, (SEQ ID NO: 3171); XM_005624533.2, TOP2A, A_8, Del, 13, essential, KGLHQKEPKRIQA, (SEQ ID NO: 3172); XM_005617468.2, BDP1, A_8, Del, 14, essential, high expression, KLEETTLPERRYQK, (SEQ ID NO: 3173); XM_005624952.2, PAFAH1B1, A_8, Del, 14, essential, high expression, NGHLLLDYKRRLWN, (SEQ ID NO: 3174); XM_005631997.2, PRPF40A, A_8, In, 14, essential, high expression, NLYLEYKGGSKASI, (SEQ ID NO: 3175); XM_535848.5, SMC4, A_8, Del, 14, essential, high expression, NLAGFQEYMADWGI, (SEQ ID NO: 3176); XM_005630347.2, CEBPZ, A_8, Del, 14, essential, high expression, KLRQKKLCLTVPVL, (SEQ ID NO: 3177); XM_014106985.1, ZMYND8, A_8, Del, 14, essential, high expression, KSPNWPTQWRLRRS, (SEQ ID NO: 3178); XM_014107402.1, ZMYM2, A_8, In, 14, high expression, KGSQEKSCIRIPVS, (SEQ ID NO: 3179); XM_005631775.2, ZNF330, A_8, In, 14, high expression, KDWCEEEGREPSGT, (SEQ ID NO: 3180); XM_014109213.1, CSPP1, A_8, In, 14, high expression, KCNYGVIRNEKTAS, (SEQ ID NO: 3181); XM_861706.4, FMR1, A_8, In, 14, high expression, TYRYKGKQHSFFST, (SEQ ID NO: 3182); XM_005639749.2, FXR1, A_8, Del, 14, high expression, KIPSLNAQWMFLRI, (SEQ ID NO: 3183); XM_003640176.3, ITGA6, A_8, Del, 14, high expression, NSGSQVGMKMKATL, (SEQ ID NO: 3184); XM_014119005.1, MPP6, A_8, In, 14, high expression, KEKDDVSHNQKCRI, (SEQ ID NO: 3185); XM_531808.5, RHOQ, A_8, In, 14, high expression, KNRIEMYKLLFDYV, (SEQ ID NO: 3186); XM_003435295.1, SLFN5, A_8, In, 14, high expression, KHLPGSDLENLHEK, (SEQ ID NO: 3187); XM_014107325.1, BRCA2, A_8, Del, 14, driver, essential, KTVYRMTQKNQLCL, (SEQ ID NO: 3188); XM_005623009.2, RBBP8, A_8, In, 14, essential, KPPENSTLQQHFYF, (SEQ ID NO: 3189); XM_005623419.2, WDHD1, A_8, In, 14, essential, RRGRFQKRAECWLQ, (SEQ ID NO: 3190); XM_014111624.1, THOC2, A_8, In, 15, essential, high expression, RETGQFRRKGREETS, (SEQ ID NO: 3191); XM_005627403.2, HSP90AB1, A_8, Del, 15, driver, high expression, KRPRRLRKNTLIKKN, (SEQ ID NO: 3192); XM_014108478.1, KIF21A, A_8, Del, 15, high expression, NTLQLKMGTLLQTLK, (SEQ ID NO: 3193); XM_537881.5, OSBPL1A, A_8, Del, 15, high expression, KMIRKILKRRKTANR, (SEQ ID NO: 3194); XM_014122796.1, SWAP70, A_8, In, 15, high expression, KSHQESPAHYRRRCI, (SEQ ID NO: 3195); XM_005626151.2, UGP2, A_8, In, 15, high expression, NTTEVQSLSCENLHF, (SEQ ID NO: 3196); XM_535760.5, FAM162A, A_8, In, 15, high expression, SSPERGSSCEGQDRV, (SEQ ID NO: 3197); XM_014117078.1, RABGAP1, A_8, Del, 15, high expression, KTVLSLVTTSRFVLN, (SEQ ID NO: 3198); XM_005638465.2, RNF111, A_8, In, 15, high expression, KRSVSPKKICIAPQF, (SEQ ID NO: 3199); XM_005624533.2, TOP2A, A_8, In, 15, essential, KGCTKRNQKGSRLKF, (SEQ ID NO: 3201); XM_005637237.2, CHD4, A_8, In, 16, driver, essential, high expression, KYQTTFHVQYCRWWFY, (SEQ ID NO: 3202); XM_005617468.2, BDP1, A_8, In, 16, essential, high expression, NWKRQLSQREDTRNNG, (SEQ ID NO: 3203); NM_001003327.2, HSP90B1, A_8, Del, 16, essential, high expression, NQKPKKLRKLSGIGSL, (SEQ ID NO: 3204); XM_014109172.1, PRKDC, A_8, Del, 16, essential, high expression, KTLKRCMKECMQLWEI, (SEQ ID NO: 3205); XM_005640282.2, LRP2, A_8, Del, 16, high expression, KSSKLARNQIAVSHQQ, (SEQ ID NO: 3207); XM_014122790.1, NUCB2, A_8, In, 16, high expression, IRTRNSSIRAWWKIEV, (SEQ ID NO: 3208); XM_014120114.1, SORBS2, A_8, In, 16, high expression, ITNSGTLSTTQKRELL, (SEQ ID NO: 3209); XM_005618393.2, BTBD1, A_8, In, 16, high expression, TDPGTKRYWIQLRWNC, (SEQ ID NO: 3210); XM_536616.5, GABARAP, A_8, Del, 16, high expression, KSIWCLLISQLVSSTS, (SEQ ID NO: 3211); XM_547684.5, SPIRE1, A_8, Del, 16, high expression, KVLTKSSWTSSDQDPL, (SEQ ID NO: 3212); XM_014112788.1, LSM11, A_8, Del, 16, essential, KSESPKWITSRYSLDT, (SEQ ID NO: 3213); XM_014121374.1, NAA15, A_8, In, 16, essential, KCRKRKAAEKSEKEKG, (SEQ ID NO: 3214); XM_534728.4, THOC5, A_8, Del, 16, essential, KLQIQPISISLIKLAS, (SEQ ID NO: 3215); XM_014110313.1, TRIO, A_8, Del, 16, essential, KRASRCQDSCLRTVSR, (SEQ ID NO: 3216); XM_003433496.3, HOXC11, A_8, In, 16, driver, TEQRPAAVFLGKSSAV, (SEQ ID NO: 3217); XM_014111157.1, COPA, A_8, In, 17, essential, high expression, KLVPWCSGVRRERNNWG, (SEQ ID NO: 3218); XM_003639706.3, RIF1, A_8, Del, 17, essential, high expression, KKDVRKKKRPFRKVHCM, (SEQ ID NO: 3219); XM_535848.5, SMC4, A_8, In, 17, essential, high expression, IWQDSRNIWQIGGFRSN, (SEQ ID NO: 3220); XM_005620224.2, USP1, A_8, In, 17, essential, high expression, KCRSYWTSWRTKEQSRL, (SEQ ID NO: 3221); XM_005627049.2, BRD2, A_8, In, 17, essential, high expression, REKEETEGREASRPSWG, (SEQ ID NO: 3222); XM_014118978.1, AKAP9, A_8, Del, 17, driver, high expression, KRGRLQAVNMMCQHTML, (SEQ ID NO: 3223); XM_005639464.1, BBX, A_8, Del, 17, high expression, NSTAFLNIVLLHLTGNA, (SEQ ID NO: 3224); XM_005618326.2, CHD2, A_8, In, 17, high expression, SESQKTCPQKNSAQTSS, (SEQ ID NO: 3225); XM_014118082.1, DST, A_8, In, 17, high expression, RSCVFLSRTDGCFSPPC, (SEQ ID NO: 3226); XM_532048.4, RAB14, A_8, Del, 17, high expression, NLWLIVLTQLVLNLVQE, (SEQ ID NO: 3227); XM_005638906.2, TTC3, A_8, In, 17, high expression, RSKKISTRKNGRGPKGK, (SEQ ID NO: 3228); XM_845089.4, CXCL13, A_8, In, 17, high expression, KCYFNSTSSSAEEKNQL, (SEQ ID NO: 3229); XM_533539.4, KIAA0020, A_8, In, 17, high expression, KVHRKRCKDITRKKKIS, (SEQ ID NO: 3230); XM_003432196.3, MAP4K3, A_8, Del, 17, high expression, KDLLLRSYYSILLSHNL, (SEQ ID NO: 3231); XM_005635554.2, SKA3, A_8, In, 17, essential, KFSGYHENKRVFPEVWI, (SEQ ID NO: 3232); XM_538859.5, WDR46, A_8, In, 17, essential, NLVKRGGKFLEKGPPVK, (SEQ ID NO: 3233); XM_005641516.2, ATRX, A_8, In, 18, driver, essential, high expression, NKEISRFGMHSSTLHGPW, (SEQ ID NO: 3234); XM_005631997.2, PRPF40A, A_8, Del, 18, essential, high expression, KPILGIQRRKQSKHLKNY, (SEQ ID NO: 3235); XM_014120762.1, NAMPT, A_8, Del, 18, essential, high expression, NGVLKILPLVLVELCYRS, (SEQ ID NO: 3236); XM_014113449.1, CUL5, A_8, Del, 18, high expression, KITVVENYIGIISCQMEL, (SEQ ID NO: 3237); XM_005634532.2, TFDP2, A_8, In, 18, high expression, RNQVDWLAYQFCSGMSKS, (SEQ ID NO: 3238); NM_001003175.2, VEGFA, A_8, In, 18, high expression, INSRKGEGAKKKAQEIPV, (SEQ ID NO: 3239); XM_014120252.1, SLC4A1AP, A_8, In, 18, high expression, SSWSRQTSSNTFLQISRR, (SEQ ID NO: 3240); XM_014110770.1, UBR3, A_8, In, 18, high expression, NHCSREENTGQRRKATKG, (SEQ ID NO: 3241); XM_848505.4, TWISTNB, A_8, Del, 18, essential, KRRRKTQSPMKWRVITES, (SEQ ID NO: 3242); XM_005615312.2, ZNF407, A_8, Del, 18, essential, NLLRKWFPLIRKENLLSP, (SEQ ID NO: 3243); XM_005620587.1, ZCCHC14, A_8, Del, 18, essential, KMKDMLNVPLRSCGLILQ, (SEQ ID NO: 3244); XM_014113891.1, CAMTA1, A_8, In, 18, driver, ISAEPACCCADPEVLPEL, (SEQ ID NO: 3245); XM_005627570.2, IBTK, A_8, Del, 19, high expression, NLLLAIVQEIMSKKFVLKE, (SEQ ID NO: 3246); XM_014121882.1, MAP4, A_8, In, 19, high expression, KEETKAKEISSSPGWGTLG, (SEQ ID NO: 3247); XM_005638144.2, MTDH, A_8, Del, 19, high expression, KRKRRSKVKITLLHRTQKN, (SEQ ID NO: 3248); XM_005635021.2, SAMHD1, A_8, Del, 19, high expression, KSNTVIYTSMWVRLSQREK, (SEQ ID NO: 3249); XM_544672.5, SECISBP2L, A_8, Del, 19, high expression, NYRKLYQRQLEKRIKPLCS, (SEQ ID NO: 3250); XM_014120114.1, SORBS2, A_8, Del, 19, high expression, NHQLRHPFHHPEKRAFVAP, (SEQ ID NO: 3251); XM_014120778.1, CACNA2D1, A_8, Del, 19, high expression, KIERKILHCCGRCLAVPQA, (SEQ ID NO: 3253); XM_005616873.2, CD3EAP, A_8, In, 19, essential, KAGAKRTRGFEASSSRSPP, (SEQ ID NO: 3254); XM_535194.5, KIN, A_8, Del, 19, essential, KRSRTLMMRKKQPNLLKNK, (SEQ ID NO: 3255); XM_005635554.2, SKA3, A_8, Del, 19, essential, KMMPTLQILPWHLHSALLV, (SEQ ID NO: 3256); XM_847012.4, PDCD1LG2, A_8, In, 19, driver, KHNKKLYHRRKEGSGRSYL, (SEQ ID NO: 3257); XM_005616941.2, CUL2, A_8, Del, 20, essential, high expression, KMTWQICMSYFVLCPLVYLI, (SEQ ID NO: 3258); XM_014118082.1, DST, A_8, Del, 20, high expression, KKLCLLVKNRWMLSKSLLSH, (SEQ ID NO: 3259); XM_535698.4, LARP7, A_8, In, 20, high expression, NDIPNEIRVKNGNKWSTYYI, (SEQ ID NO: 3260); XM_005634532.2, TFDP2, A_8, Del, 20, high expression, KKSSGLACLPILLRNVKILR, (SEQ ID NO: 3261); XM_005638394.2, CEP152, A_8, Del, 20, essential, KLSVICFVIFKRVRKELQKW, (SEQ ID NO: 3262); XM_534660.5, VPS33A, A_8, In, 20, essential, TSARRKPSHSDIFPRGCNLC, (SEQ ID NO: 3263); NM_001005760.1, LIFR, A_8, In, 20, driver, IFNHRSHSFKGTRYLERVEF, (SEQ ID NO: 3264); XM_014111626.1, THOC2, A_8, Del, 21, essential, high expression, KRNGTVQEERKRRNSPRTSTD, (SEQ ID NO: 3265); XM_014111626.1, THOC2, A_8, In, 21, essential, high expression, RETGQFRRKGREETVLGQAQI, (SEQ ID NO: 3266); NM_001145981.1, MPHOSPH8, A_8, Del, 21, high expression, KMNPKRKTRKGMIWTRKKKAV, (SEQ ID NO: 3267); XM_005634301.2, NKTR, A_8, Del, 21, high expression, KITLQVRGTAAALKRDFTINM, (SEQ ID NO: 3268); XM_014120122.1, SORBS2, A_8, Del, 21, high expression, KIRPPKNRLRRQNVKHPCLRP, (SEQ ID NO: 3269); XM_544679.5, TMOD3, A_8, In, 21, high expression, RENIYPETETYTDVHRRKSMS, (SEQ ID NO: 3270); XM_005618393.2, BTBD1, A_8, Del, 21, high expression, NRPWDKTILDSVAMELLTHSG, (SEQ ID NO: 3271); XM_014121533.1, TAMM41, A_8, In, 21, essential, KLEPLLFPESFGAQDYHHCPE, (SEQ ID NO: 3273); XM_005634505.2, COPB2, A_8, Del, 22, essential, high expression, NHLNQILELKVSMEASYWESDL, (SEQ ID NO: 3274); XM_014111529.1, SMC1A, A_8, Del, 22, essential, high expression, KTSRLNARKPNRRKKRLTATSA, (SEQ ID NO: 3275); XM_531830.5, MTIF2, A_8, In, 22, essential, high expression, RTKTFKAKRERRKGFTCTSYNY, (SEQ ID NO: 3276); XM_014112562.1, FAM193A, A_8, Del, 22, high expression, KTLQKRNACTISKMLLWKRTKL, (SEQ ID NO: 3277); XM_005615362.2, LMAN1, A_8, Del, 22, high expression, KRRNSRRAILTFSGILTKYLRV, (SEQ ID NO: 3278); XM_014107402.1, ZMYM2, A_8, In, 22, high expression, KSFNNGKQSMSSIPSVFLMWNR, (SEQ ID NO: 3280); XM_005641771.2, THOC2, A_8, In, 23, essential, high expression, RETGQFRRKGREETISFSSYWSF, (SEQ ID NO: 3283); XM_537461.5, ARID4A, A_8, In, 23, high expression, RLRRINGRGFKSRSRNAFSGSEE, (SEQ ID NO: 3284); XM_014115338.1, ASH1L, A_8, Del, 23, high expression, KGKEEKLGGLKWWQEAHAGLQKG, (SEQ ID NO: 3285); XM_014108254.1, LMAN1, A_8, Del, 23, high expression, KRRNSRRAILTFSGILQTKYLRV, (SEQ ID NO: 3286); XM_014112788.1, LSM11, A_8, In, 23, essential, KAKAQSGLPAGIHSTHKSDIHSR, (SEQ ID NO: 3287); XM_014116241.1, RNF213, A_8, Del, 24, driver, high expression, KTSTTFSTRKALWKAPLRNAFSIS, (SEQ ID NO: 3289); XM_005615655.2, AKAP7, A_8, In, 24, high expression, KAKRLSTQLFPIHSDHQQRDYERN, (SEQ ID NO: 3290); XM_005629478.2, LUC7L2, A_8, In, 24, high expression, KRSRGSLSEFYASFQFPTTETSSL, (SEQ ID NO: 3291); XM_534964.5, KIF11, A_8, In, 24, essential, NPFTGTLPVNRSLGRTILCLGGKV, (SEQ ID NO: 3292); XM_547304.5, BCL10, A_8, Del, 24, driver, KYSVEKTLKKFLAEHQAGKGPENC, (SEQ ID NO: 3293); XM_014120072.1, WRN, A_8, Del, 24, driver, NWKELHSRENFLNGCLCRMRHVLQ, (SEQ ID NO: 3294); XM_014112366.1, MPHOSPH10, A_8, In, 25, essential, high expression, TLAASRGSDSSKATREQPSRGDSAL, (SEQ ID NO: 3295); XM_014110834.1, CWC22, A_8, Del, 25, essential, high expression, KREEREEILFPKMSTDSEIRTVKML, (SEQ ID NO: 3296); XM_014121441.1, LARP1B, A_8, Del, 25, high expression, NSDKKFLKISKKKPKKTTNLVSCMD, (SEQ ID NO: 3297); XM_544679.5, TMOD3, A_8, Del, 25, high expression, KGKYLSRNRNLYRRSQKKKYVLIQN, (SEQ ID NO: 3298); XM_014112635.1, LIAS, A_8, In, 25, essential, KGILTKWTRPSRFCIWRSCRQEQVG, (SEQ ID NO: 3299); XM_849421.4, YY1, A_8, Del, 25, essential, KILTMRQWLKSRSLERTPLLIIQST, (SEQ ID NO: 3300); XM_547682.5, AFG3L2, A_8, In, 26, essential, high expression, RIKASCYPEPFWRRNWWQWKTRWQER, (SEQ ID NO: 3301); XM_005619358.2, ZNF131, A_8, Del, 26, essential, high expression, KERLQKLQMLSLSHCHLQNQNLLKLR, (SEQ ID NO: 3302); XM_005623762.2, DDX24, A_8, In, 26, essential, high expression, KERKKKIGAFPGYCSKGAQKSKDMDA, (SEQ ID NO: 3304); XM_005623082.2, ANKRD12, A_8, Del, 26, high expression, KMTIERKVEKRWIENMTKKNLKKTGI, (SEQ ID NO: 3306); XM_848505.4, TWISTNB, A_8, Del, 26, essential, KRKENTVRKLNLPHFWNIHLKRKGES, (SEQ ID NO: 3307); XM_844731.4, UBE4B, A_8, Del, 27, essential, high expression, KHQRCAASQQSASFSATSAHSAYPILR, (SEQ ID NO: 3308); XM_546032.4, CETN3, A_8, Del, 27, high expression, KEENSLKNRNKKLKMLLNCLTQTKMKQ, (SEQ ID NO: 3309); XM_538754.4, MURC, A_8, In, 27, high expression, KQIPCGDLPGGDSVSNIPVYCQGQKPN, (SEQ ID NO: 3310); XM_546202.5, DCP2, A_8, Del, 27, essential, NHHENFRIILKQMLYMTCLAPVKTSC, (SEQ ID NO: 3311); XM_014113954.1, ZCCHC14, A_8, Del, 27, essential, KMKDMLNVPLSYPSCTLKKIWRSSFLA, (SEQ ID NO: 3313); XM_014111218.1, ZC3H11A, A_8, In, 28, high expression, NEGKIQEARRRFFWSFQSFTPTSAHSRS, (SEQ ID NO: 3314); XM_005630261.2, EIF2B4, A_8, Del, 28, essential, NSRRRSGRRKREQNQKKLPLLYLQPSSK, (SEQ ID NO: 3315); XM_014106743.1, XRN1, A_8, Del, 29, essential, high expression, KMRRYLDILRKLEVSGCIHLQQSSFLQST, (SEQ ID NO: 3316); XM_533976.5, TAF1D, A_8, Del, 29, high expression, KRNVKRGNISQKKDQGEDQKEEKPLDAPK, (SEQ ID NO: 3317); XM_014115839.1, BAZ1A, A_8, In, 29, high expression, SKTESSEFSRTTNYSSFILDQPYPSFTLA, (SEQ ID NO: 3318); XM_014120778.1, CACNA2D1, A_8, In, 29, high expression, KSRGRSFIVVAGVWQCHRPSPILSSFSMG, (SEQ ID NO: 3319); XM_536500.4, NUP155, A_8, Del, 29, essential, KILRVLGFISINMENQKKTSWDCRLSKKD, (SEQ ID NO: 3320); XM_005618621.2, RFC1, A_8, Del, 29, essential, KTKEKEKLAQLRRNQNLKEVDLLPRRTAL, (SEQ ID NO: 3321); XM_542572.5, ZC3H13, A_8, Del, 30, essential, high expression, NVKNPKAILIFLMKKQPCRIKRREVHGLPL, (SEQ ID NO: 3322); XM_005635475.2, PDS5B, A_8, Del, 30, high expression, KVTRETTLIFLNWTNLEAGKKHLSQIQKRN, (SEQ ID NO: 3323); XM_533087.4, PNPLA8, A_8, Del, 30, high expression, KRKINISRKNQNLKIKRLKKRKQALQILAS, (SEQ ID NO: 3324); XM_543181.4, CENPJ, A_8, Del, 30, essential, KLVVLQKWLILKKDLLKLQSEKGNRHLKTT, (SEQ ID NO: 3325); XM_005627814.2, POP1, A_8, In, 30, essential, KGRMESRSRGHVSGDSQVYHCFYFCSGPSC, (SEQ ID NO: 3326); XM_005622133.1, ZRANB2, A_8, Del, 31, essential, high expression, KDEQDHGHPKGTTGHLLDHPILVPVQVQKRN, (SEQ ID NO: 3327); XM_005622133.1, ZRANB2, A_8, In, 31, essential, high expression, KTNKITVTRKAPQVIFWIIPFWFPFKFKKEI, (SEQ ID NO: 3328); XM_005636747.2, CBX5, A_8, Del, 31, high expression, KRESRAMISLGALREDWNQKRSLGRQIPVVI, (SEQ ID NO: 3330); XM_005629478.2, LUC7L2, A_8, Del, 31, high expression, KEKQRKFIGILCQLPVSNNRNFESVKSALPI, (SEQ ID NO: 3331); XM_844680.4, TM9SF3, A_8, Del, 31, high expression, KVSVITMKLWEKHFKELNWNLVAWILNSKRM, (SEQ ID NO: 3333); XM_005633326.2, CEP57, A_8, Del, 31, essential, KRSQNHLKRKVLGTILLYNHIIDYAWGICRL, (SEQ ID NO: 3334); XM_014112635.1, LIAS, A_8, Del, 31, essential, KRNSYKMDQTFKILYLEILQTRASGMNIKET, (SEQ ID NO: 3335); XM_533327.6, DDX18, A_8, In, 32, essential, high expression, KEIYHNSQWRSRNTASQFRIKKKEEEKEKNGG, (SEQ ID NO: 3336); XM_005627838.2, UBRS, A_8, Del, 32, driver, high expression, KCWRKLEQKIKSPNLALVFLQCQTSLLVPRYA, (SEQ ID NO: 3337); XM_862709.4, FBXO38, A_8, Del, 32, high expression, KTRMFIPAAAAPPPAQWEPPAHTALLLKAPTL, (SEQ ID NO: 3338); XM_005619248.2, CLINT1, A_8, In, 32, high expression, KLEKSLQVIAASSLPHKEWIRACCYKCQRTHL, (SEQ ID NO: 3339); XM_014109179.1, ATP8B1, A_8, Del, 33, high expression, KMILFQLTSCYCPALSLTASAMSKQLNWMEKPI, (SEQ ID NO: 3340); XM_014107402.1, ZMYM2, A_8, Del, 33, high expression, KREPREKLYQDTSLMMIVLTIQNVPFLSNIRMV, (SEQ ID NO: 3342); XM_005634454.2, CEP63, A_8, Del, 33, high expression, KTMTGSLNQHTTEHLSSRIQSSSQPMARTDMME, (SEQ ID NO: 3343); XM_005634201.2, CLASP2, A_8, Del, 33, high expression, KWVLICLGLYRQKFRKPLMLQENLFQMIFSLIF, (SEQ ID NO: 3344); XM_546411.5, SRPR, A_8, Del, 33, high expression, KRGPRRRALMALWLRAKQFLPKSQVSQRGQRTG, (SEQ ID NO: 3345); XM_542577.3, NUFIP1, A_8, Del, 33, essential, KRKERNQFFIVFVIPVIVVLKIKKSMINTCLNT, (SEQ ID NO: 3346); XM_005626702.1, TOPORS, A_8, Del, 34, high expression, KIITVKGSITTMKGTDQEACPVIDQKLHLQGLTG, (SEQ ID NO: 3347); XM_544656.5, EIF3J, A_8, In, 35, essential, high expression, RGSRSKTRSKSFRKEKNSRENKRERTATEEKARRN, (SEQ ID NO: 3348); XM_005623762.2, DDX24, A_8, Del, 35, essential, high expression, KRKEKKNWSLPRVLLQRCPKKQRHGCLKCMTIRQM, (SEQ ID NO: 3349); XM_005617306.2, DIAPH1, A_8, In, 35, high expression, ICAKEKSKRVKGVGFKDGPESLHLSGFLSHGLSRD, (SEQ ID NO: 3350); XM_547684.5, SPIRE1, A_8, In, 35, high expression, KCSRNHPGLHQIKTPFKAGCSQKTETHSTTTTEPP, (SEQ ID NO: 3351); XM_014120252.1, SLC4A1AP, A_8, Del, 36, high expression, KLLVQANFQQHFPPNIQKMTQTTVCGSHLKVVISAS, (SEQ ID NO: 3352); XM_005627814.2, POP1, A_8, Del, 36, essential, KREDGKQVPRARLRRFSPISLLLLLLRPELLKSVLC, (SEQ ID NO: 3353); NM_001110501.1, VEGFA, A_8, In, 37, high expression, INSRKGEGAKKKAQEIPSLWALLRAEKAFVCTRSADV, (SEQ ID NO: 3354); XM_535265.5, RAD17, A_8, Del, 37, essential, KLIRFLKTKKSKLLVAKMFLSFSSEHWGKYYIVKEHL, (SEQ ID NO: 3355); XM_005619382.2, NIPBL, A_8, Del, 38, essential, high expression, KSLILSFQRVKQNKMKVDWQNLNQMKTDWWRQNQVKVS, (SEQ ID NO: 3356); XM_005615655.2, AKAP7, A_8, Del, 38, high expression, KSKKVINPTISYPFRSPTKRLREELRSCKMQYSSKMSD, (SEQ ID NO: 3357); XM_532916.5, SLC4A1AP, A_8, Del, 39, high expression, KLLVQANFQQHFPPNIQKMTQTTVCGSHLKAKVEMAGPI, (SEQ ID NO: 3359); XM_014120253.1, SLC4A1AP, A_8, Del, 40, high expression, KLLVQANFQQHFPPNIQKMTQTTVCGSHLKVGQVGTPWFF, (SEQ ID NO: 3360); XM_005619838.2, CWF19L2, A_8, Del, 41, essential, high expression, KRVRNRSMKKAMSQQTVHQALKMSGLRLSHRRLLAGKRPGK, (SEQ ID NO: 3361); XM_853836.4, TCERG1, A_8, Del, 41, essential, high expression, KSLRKILGALSSPPATGKNKESLKNTSETNTSQPKLTSGRF, (SEQ ID NO: 3362); XM_014112562.1, FAM193A, A_8, In, 41, high expression, KRCKKEMLVQFPRCFYGSEQSCDGHVISHLLCVLHSHYGAV, (SEQ ID NO: 3363); XM_857314.4, PHF20L1, A_8, Del, 42, high expression, KMKLTLAVLPTLRNLHCYPQLCLQGRLAARNANMNLEILLGV, (SEQ ID NO: 3364); XM_014120446.1, EML4, A_8, Del, 43, driver, KRNLILMIKVHKFEHHLLPSPLHSLSKYTDKLKKARILLPPKA, (SEQ ID NO: 3366); XM_005625788.2, ARFGAP3, A_8, In, 44, high expression, KDRVGETWHGIWKLQKWYFTFRDFRYADHRTGNTHRSKAQKEVQ, (SEQ ID NO: 3368); XM_014112829.1, JMJD1C, A_8, Del, 45, high expression, KKTKNLPLKSKLKKRENKTILILQIAEHHLLHPRIMNRVQLYEIC, (SEQ ID NO: 3369); XM_014117718.1, VDAC1, A_8, Del, 45, high expression, KMLKSRQGISGSTSTWAVMWILTLLVLPSGVLWCWAMRAGWLATR, (SEQ ID NO: 3370); XM_014116993.1, NUP214, A_8, Del, 45, driver, essential, KSFLAHHFMSQIILSEFWMCCGSVLMSSQLCMLLRMGPWKLLQMW, (SEQ ID NO: 3371); XM_014115201.1, KIFAP3, A_8, Del, 46, high expression, KKNQASLKIHLLLKEWRLMKLLTLMTWMNTLSYYMKIFLTKFGVLL, (SEQ ID NO: 3372); XM_005634774.2, ESF1, A_8, Del, 47, essential, high expression, KTLFGIVQTHVPKENSAQETWASLKLQNVPRSSTLRQEDKCNQWFHY, (SEQ ID NO: 3373); XM_532580.5, EPS15, A_8, Del, 48, driver, high expression, KMCLRKYRSKVKMYPQHCHQRLELQQDPVHHHLGKDPSTNWILLILLN, (SEQ ID NO: 3374); XM_014121374.1, NAA15, A_8, Del, 48, essential, KMQKKKSSREIRKRKRMMMMKKSEVQKKNLSLRNWPRLKLHWKKLLNF, (SEQ ID NO: 3375); XM_005626023.2, EIF5B, A_8, Del, 51, essential, high expression, KIKKISQVLMWRVGMKMMIPPSKLRRWPKRRQKRKSVKEKREMKKKQNYGS, (SEQ ID NO: 3378); XM_014120797.1, CBLL1, A_8, Del, 51, essential, high expression, KEIRCVQAVVILCSESSSVHEVLSSCVALFKGAREHTCLSETYRLISTTAI, (SEQ ID NO: 3379); XM_014116982.1, TTF1, A_8, Del, 51, essential, KREKFHTTRNLRHWLGLVVSRMCTPKDGKGSVKLGPQKNGKGSVKLGLLVE, (SEQ ID NO: 3380); XM_005637237.2, CHD4, A_8, Del, 52, driver, essential, high expression, KISNNVSCSILQMVVLLSCTPFGRMRSGQPQSLRRLTRFGIGGTTTGCWLAS, (SEQ ID NO: 3381); XM_543181.4, CENPJ, A_8, Del, 52, essential, KLSLIIEKMYPSVQNLVILVAKSGIRHKVKTNFLFRQDWSAACLLVAPKMRP, (SEQ ID NO: 3382); XM_005630260.2, EIF2B4, A_8, In, 52, essential, TAEEEAEGGKGSRTRKNCPCCTCSPAARPSQRTARTRQSVGHCWGESSNWSK, (SEQ ID NO: 3383); XM_014107179.1, AURKA, A_8, Del, 53, essential, KGNGLWKILKLVAHWVKESLVMFTWQGKNKASLSWLLKYYLKLNWRKQELNIS, (SEQ ID NO: 3385); XM_005630261.2, EIF2B4, A_8, In, 53, essential, TAEEEAEGGKGSRTRKNCPCCTCSPAASRPSQRTARTRQSVGHCWGESSNWSK, (SEQ ID NO: 3386); NM_001110501.1, VEGFA, A_8, Del, 55, high expression, NQFEERGRGKKESARNPVPVGLAQSGESICLYKIRRRVNVPAKTQTRVARRGSLS, (SEQ ID NO: 3387); XM_014116982.1, TTF1, A_8, Del, 56, essential, KSIRKKREKFHTTRNLRHWLGLVVSRMCTPKDGKGSVKLGPQKNGKGSVKLGLLVE, (SEQ ID NO: 3389); XM_537881.5, OSBPL1A, A_8, Del, 57, high expression, NDLRKNKEQPAKTDPSRRRTGRRGGSTKAPTPTMEHRTGSTRAATGTETISICLTFI, (SEQ ID NO: 3390); XM_005640290.2, PPIG, A_8, Del, 57, high expression, KEKRNIGKIPENTRKKRRSERKARKVHLVKVRLKILKHNPSLLSVQKRSLLYLKIDS, (SEQ ID NO: 3391); XM_014120832.1, YAE1D1, A_8, In, 58, essential, KSQLPHILPIRDRSFQRFFSGNKFPLEGEGFPTHTKTHTPTPRSRVARSSSDCARPAP, (SEQ ID NO: 3393); XM_005620105.2, NCOR1, A_8, Del, 61, driver, high expression, KKKKRKMKKKKMKRRTLKRTPRKRTRRKVQQKKRKRENRPRPGDERPPTVRAAERAGSPGP, (SEQ ID NO: 3394); XM_542610.5, DIS3, A_8, Del, 63, essential, KFMKNSQNMPCFENTLLLLHQIMKFLLRQLNPRIWKLKLIQPNPWLTLWTGRIPRLSHISTLS, (SEQ ID NO: 3396); XM_844731.4, UBE4B, A_8, In, 69, essential, high expression, STKDVQPASSQPASQQHPLTVHIPYCVSPTRFPHTTKVHAAAILPGAVYALQESPIWLHAGTGENHS PG, (SEQ ID NO: 3397); XM_544717.5, HERC1, A_8, Del, 70, high expression, KALWSWLMPWQPAASPPGCPHSIGNGLLNSLCALLLRMTATIKLLPKHSPIWEEISENAPLSSWRLI RTG, (SEQ ID NO: 3398); XM_005630260.2, EIF2B4, A_8, Del, 70, essential, NSRRRSGRRKREQNQKKLPLLYLQPSSKAQPKNCQDQAVSWPLLGRKFQLVEVKLNFVLNGGLSR RLSGP, (SEQ ID NO: 3399); XM_014119900.1, WDR60, A_8, Del, 72, high expression, KTPSRPMFSTMRIMLTETSRQKRLRPGGCGRSTPERARLCQGGAGVEMSVRWPQPRRSTRPGWPAS FALLAR, (SEQ ID NO: 3400); XM_005631775.2, ZNF330, A_8, Del, 72, high expression, KRLVRGRRQRAVGNVKNSSEHREAPSIWLNTHAMHQWNVTSVRGGRRIERFATSATLYRSYQFVH SVGKRSA, (SEQ ID NO: 3401); XM_005626022.2, EIF5B, A_8, Del, 84, essential, high expression, KRKDKKAKNKVLMIMIVKNGKIKIQNQKRLQSQKWKCTLGVMMMMMILINFLKKLKGKCRNQIK GGMGQKKMRITVKELKSVPE, (SEQ ID NO: 3403); XM_014118258.1, OGFRL1, A_8, Del, 85, high expression, KQHQEDLERKQTVLAQSPAVKLPSQETPRTVMLKIQILNWKKQSPIPQRGRRLQLLLKKMKKVKII RKTVKILELQVPMMMYNYS, (SEQ ID NO: 3404); XM_547682.5, AFG3L2, A_8, Del, 87, essential, high expression, KNQSQLLPRALLEEELVAVENEVARKMILTGGPDSRRVTFHGMTRNSGCTFSGLLYFGVESCFTSCS RALGEKSRGRTLSITIFPKE, (SEQ ID NO: 3405); XM_005617468.2, BDP1, U_8, In, 8, essential, high expression, FSHQHGRN, (SEQ ID NO: 3407); XM_014110313.1, TRIO, U_8, In, 8, essential, FRRVGEVP, (SEQ ID NO: 3408); XM_546489.5, DPAGT1, U_8, In, 9, essential, FHHLGIALP, (SEQ ID NO: 3409); XM_005638158.1, KIAA1429, U_8, Del, 10, essential, high expression, LCRNMKQQIC, (SEQ ID NO: 3410); XM_005635688.1, PPP2R2A, U_8, Del, 10, essential, high expression, FLKSSPRFPM, (SEQ ID NO: 3411); XM_544113.5, PREX2, U_8, Del, 10, driver, FRMRKWKDQI, (SEQ ID NO: 3412); XM_535242.6, PLK2, U_8, In, 11, high expression, FAGLHSRQTLF, (SEQ ID NO: 3413); NM_001003232.1, CANX, U_8, Del, 11, high expression, LTTLLFVGIEE, (SEQ ID NO: 3414); XM_014109046.1, CSGALNACT2, U_8, Del, 11, high expression, LRRQTLWNIDM, (SEQ ID NO: 3415); XM_005634758.2, DSTN, U_8, In, 11, high expression, FVGTRTSSSEK, (SEQ ID NO: 3416); XM_005621511.2, SMG1, U_8, Del, 11, essential, FILIAKLAKTG, (SEQ ID NO: 3417); XM_003639706.3, RIF1, U_8, In, 12, essential, high expression, FQTCKYTYHCCS, (SEQ ID NO: 3418); XM_014114821.1, ABCD3, U_8, In, 12, high expression, FKTHTDSENHGP, (SEQ ID NO: 3419); XM_534660.5, VPS33A, U_8, In, 12, essential, CQTQARTNGYNC, (SEQ ID NO: 3420); XM_005618433.2, MORF4L1, U_8, In, 13, high expression, FFFFFFFFFYSYR, (SEQ ID NO: 3421); XM_859725.4, WAPL, U_8, Del, 13, essential, FSPPTENMKQVSK, (SEQ ID NO: 3422); XM_005638410.2, DMXL2, U_8, In, 14, high expression, FELCDVQFSMGPSR, (SEQ ID NO: 3423); XM_005638410.2, DMXL2, U_8, In, 14, high expression, FRYTNTGAPRSKFL, (SEQ ID NO: 3424); XM_005638907.2, TTC3, U_8, Del, 15, high expression, FLVMEPLLRSLILAS, (SEQ ID NO: 3425); XM_005633659.2, TMEM41B, U_8, In, 15, essential, YWDFSRCCTSLFCSN, (SEQ ID NO: 3426); XM_005634584.2, MBNL1, U_8, Del, 17, essential, high expression, FLFFLSFSFSFFFPIVW, (SEQ ID NO: 3427); XM_005638410.2, DMXL2, U_8, Del, 21, high expression, FQIHKYRSTKIQILIAGLFYI, (SEQ ID NO: 3428); XM_533302.6, EXOSC9, U_8, Del, 22, essential, LTLNSLRWLLQLLNLAGSQISW, (SEQ ID NO: 3429); XM_005617430.2, IPO11, U_8, Del, 22, essential, FLHFLMRWHINLIRRWTSFWEI, (SEQ ID NO: 3430); XM_003639706.3, RIF1, U_8, Del, 25, essential, high expression, FPNMQIHLSLLFMIALLRLEKMLLM, (SEQ ID NO: 3431); XM_546489.5, DPAGT1, U_8, Del, 28, essential, FSPPWDCSTITGTHHRCLWEIPSVTLLA, (SEQ ID NO: 3432); XM_014106743.1, XRN1, U_8, In, 29, essential, high expression, FEEKWGSSIPAKQSWRKHDVRNFSECRIR, (SEQ ID NO: 3433); XM_005618433.2, MORF4L1, U_8, Del, 30, high expression, FLFFFFFFFLLLQVSEYCAFMGLFFMKQSV, (SEQ ID NO: 3434); XM_852029.5, HNRNPH1, U_8, Del, 40, essential, high expression, FLTAKFKMGLKVFVSSTPEKADRVARLLLNLNQKMKSNWP, (SEQ ID NO: 3435); XM_005629703.2, PAXIP1, U_8, Del, 53, essential, LESLRVSPRCRPQTGAPCGLCSLSTGATASCTSTRNAHIWLFQNQKGRNTNAR, (SEQ ID NO: 3438); XM_005629703.2, PAXIP1, U_8, In, 63, essential, WNHCVSLPGVVHRQERLVGFAHFLRGRLPAAPQQEMHTSGCSRTKRGEIRMRVKESKYQNCYP, (SEQ ID NO: 3439); XM_535242.6, PLK2, U_8, Del, 68, high expression, FCRASLQTDSLLAVVIQFQISTCQARLRISLRKQLLLFLVAKKTKQDILTHIIECLKKMKKFTSLGMI, (SEQ ID NO: 3440); XM_536172.5, TARSL2, U_8, Del, 77, high expression, FSMISVLEAAFFFPEEPSFIIHLWISYERSITDVTSQKCSLPTCSTANSGKPQATGSITARTCLPLILKRIL LLLSP, (SEQ ID NO: 3441); XM_014114200.1, SRRT, C_8, In, 8, essential, high expression, QAGQHRRD, (SEQ ID NO: 3442); XM_845787.4, PARP14, C_8, Del, 8, high expression, PASWWFSI, (SEQ ID NO: 3443); XM_014118099.1, NOL8, C_8, In, 8, essential, PEESQSAE, (SEQ ID NO: 3444); XM_537035.5, RBM15, C_8, In, 8, driver, PASATVAP, (SEQ ID NO: 3445); XM_005631207.2, SSRP1, C_8, In, 9, essential, high expression, QLRRLGIRI, (SEQ ID NO: 3446); XM_005626438.2, SRFBP1, C_8, In, 9, essential, QKSVVTGGR, (SEQ ID NO: 3447); XM_540739.5, FNBP4, C_8, In, 10, essential, high expression, PSSPSSCRRW, (SEQ ID NO: 3448); XM_014119322.1, MACF1, C_8, In, 10, high expression, PGSQTAYFSE, (SEQ ID NO: 3449); XM_005616928.2, COMMD3, C_8, Del, 10, high expression, PLPSRIIRIP, (SEQ ID NO: 3450); XM_014110156.1, PVRL3, C_8, In, 10, high expression, LASKRPSISD, (SEQ ID NO: 3451); XM_846534.4, PRCC, C_8, In, 11, driver, essential, PGNCPRCLLHR, (SEQ ID NO: 3452); XM_005631207.2, SSRP1, C_8, Del, 12, essential, high expression, PAQKTRHQDLMS, (SEQ ID NO: 3453); XM_014107801.1, CCDC92, C_8, Del, 12, high expression, PRTSCLKRPAAA, (SEQ ID NO: 3454); XM_005640282.2, LRP2, C_8, In, 12, high expression, PMQVYVRRKLLF, (SEQ ID NO: 3455); NM_001003339.1, CYP2E1, C_8, Del, 12, high expression, PITNSVSFPARA, (SEQ ID NO: 3456); NM_001003339.1, CYP2E1, C_8, In, 12, high expression, PLQTLCRSPLGL, (SEQ ID NO: 3457); XM_014112554.1, ADD1, C_8, In, 13, high expression, QHPCQAWGRRWMC, (SEQ ID NO: 3458); XM_014122376.1, POLR2E, C_8, In, 13, essential, PRTSAGGKDHPAQ, (SEQ ID NO: 3459); XM_540739.5, FNBP4, C_8, Del, 14, essential, high expression, PQNPLLPPLLPLLL, (SEQ ID NO: 3460); XM_005622233.2, HSD11B1, C_8, In, 14, high expression, PSGVLLGLLLLFCE, (SEQ ID NO: 3461); XM_854467.4, CLIP1, C_8, In, 15, driver, high expression, PLHTPRQSERGAPVL, (SEQ ID NO: 3462); XM_014106952.1, RTEL1, C_8, In, 15, essential, RGGAGTRPPHLVRPT, (SEQ ID NO: 3463); XM_014114200.1, SRRT, C_8, Del, 16, essential, high expression, PGWAASQRLTWVCHHR, (SEQ ID NO: 3464); XM_540739.5, FNBP4, C_8, In, 17, essential, high expression, PRIPSSPPSSPSSCRRW, (SEQ ID NO: 3465); XM_014118099.1, NOL8, C_8, Del, 17, essential, PRRESKCRMRHLLHLQL, (SEQ ID NO: 3466); XM_005626438.2, SRFBP1, C_8, Del, 17, essential, PKKCCYRRKINMKLIRV, (SEQ ID NO: 3467); XM_541488.5, PNKP, C_8, Del, 17, essential, PSSCPRTGKPLSWAGDP, (SEQ ID NO: 3468); XM_005619042.2, ZSWIM8, C_8, Del, 17, essential, PTLMMSNGCWGWQQSWE, (SEQ ID NO: 3469); XM_541589.4, CIC, C_8, In, 17, driver, PAIPPTPRPLHSCHRQV, (SEQ ID NO: 3470); XM_541589.4, CIC, C_8, Del, 17, driver, PSHPPHPPAPPQLPQAG, (SEQ ID NO: 3471); XM_547544.4, GON4L, C_8, Del, 18, essential, high expression, PRSPSRPGTVLSWRSCMQ, (SEQ ID NO: 3472); XM_005619266.1, LARP1, C_8, In, 18, high expression, HPSLATRDQTGACLARPG, (SEQ ID NO: 3473); XM_014115306.1, NES, C_8, In, 18, high expression, LPAPATDWEAAGSRGYTG, (SEQ ID NO: 3474); XM_005625495.2, SMARCC2, C_8, Del, 18, high expression, PLDPMAPHRSPTNKLLPQ, (SEQ ID NO: 3475); XM_005635161.2, STAU1, C_8, In, 18, high expression, PHEELCDQGVGWGVCGGR, (SEQ ID NO: 3476); XM_014111162.1, PADI2, C_8, In, 18, high expression, PGLFWEPLVPPALRSPTP, (SEQ ID NO: 3477); XM_547544.4, GON4L, C_8, In, 19, essential, high expression, PEAQADQGQYFHGEVACSR, (SEQ ID NO: 3479); XM_014119322.1, MACF1, C_8, Del, 19, high expression, PRVPDCLLLRVIYPSAAGL, (SEQ ID NO: 3480); XM_014111220.1, ELK4, C_8, Del, 19, driver, PFPPFPPLCRSPPGPPRRR, (SEQ ID NO: 3481); XM_014112554.1, ADD1, C_8, Del, 20, high expression, PAPLSSLGKEVDVLKSTCYH, (SEQ ID NO: 3482); XM_014112550.1, ADD1, C_8, In, 20, high expression, QHPCQAWGRPATGAHFRRGQ, (SEQ ID NO: 3483); XM_005636081.1, SFSWAP, C_8, In, 20, essential, PTGGRRGHLLQHPACWCGCA, (SEQ ID NO: 3484); XM_546111.5, CCDC6, C_8, Del, 20, driver, PRPARRPPPRRSRRTPPPSL, (SEQ ID NO: 3485); XM_546111.5, CCDC6, C_8, In, 20, driver, RGRRAARPLAAAGAPPLPAL, (SEQ ID NO: 3486); XM_536612.5, PELP1, C_8, In, 21, essential, high expression, PGGGAAGGGEPPSRAAASRAY, (SEQ ID NO: 3487); XM_014118314.1, BAG6, C_8, Del, 21, essential, PQPPVTRGSSEFPTRAWNPWS, (SEQ ID NO: 3489); XM_014111220.1, ELK4, C_8, In, 21, driver, HFLRFPLCAGAPQDPHAAAES, (SEQ ID NO: 3490); XM_014121888.1, MAP4, C_8, In, 22, high expression, PPPWRPQRRHPGSWLPVPGCFL, (SEQ ID NO: 3491); XM_014121888.1, MAP4, C_8, Del, 22, high expression, PTSLAAPAQAPGLLAPCPGLLS, (SEQ ID NO: 3492); XM_545182.4, ICE1, C_8, Del, 22, essential, PCHPCSLLWSLHLQGLRNQFLL, (SEQ ID NO: 3493); XM_005619266.1, LARP1, C_8, Del, 23, high expression, PPQPGNQRPNRSLPGTTRMRHRV, (SEQ ID NO: 3494); XM_014122046.1, SMARCA4, C_8, In, 23, driver, essential, PRTLRRAPWDARSAPRRASQALA, (SEQ ID NO: 3495); XM_014113123.1, FYB, C_8, In, 24, high expression, QAEAPASPVYLGPTATQTQQTSQR, (SEQ ID NO: 3496); XM_005639159.2, AFF1, C_8, In, 25, driver, high expression, PTCVILILILPEASQACTKEAKAGS, (SEQ ID NO: 3497); XM_005636081.1, SFSWAP, C_8, Del, 25, essential, PHRGSTWPLTTAPCLLVWLCLALLG, (SEQ ID NO: 3498); XM_014113123.1, FYB, C_8, Del, 26, high expression, PSRSPCLPCLPWAHRHPNPADLPTLT, (SEQ ID NO: 3499); XM_005622233.2, HSD11B1, C_8, Del, 27, high expression, PFWGSSWPTTTILRMRNSDQRCSKERK, (SEQ ID NO: 3500); XM_005621365.2, ATXN2L, C_8, In, 28, high expression, PRRGAPEWGACTLSFHTLTLSLHRTPPR, (SEQ ID NO: 3501); XM_005621364.2, ATXN2L, C_8, In, 29, high expression, PRRGAPEWGACTLSFHTLTLSLHRTPPSM, (SEQ ID NO: 3502); XM_005640282.2, LRP2, C_8, Del, 33, high expression, PHAGVCTEEIAILMRMTSPNASVPVATWENIVK, (SEQ ID NO: 3503); XM_014117150.1, SMARCC2, C_8, In, 33, high expression, PWTPWPLTVPQPTNSSRNDARGSARQRAPRRGG, (SEQ ID NO: 3504); XM_014113186.1, NCAM1, C_8, In, 33, high expression, RPAGGPRHRRPGPRARPARRRREEPGRGSRGPH, (SEQ ID NO: 3505); XM_014110156.1, PVRL3, C_8, Del, 33, high expression, PCFKKTFYIRLNTCLCRLSSKKERLAVSSTLMD, (SEQ ID NO: 3506); XM_534998.5, PPRC1, C_8, In, 34, essential, PTNSAPGIWYSWHLCCAPHLQCALGTSSSPSPTL, (SEQ ID NO: 3507); XM_846492.4, XIRP1, C_8, In, 36, high expression, PSPGRSAAATAQGVLLQVPPAETSQRAPPPLQAHPP, (SEQ ID NO: 3509); XM_005619566.2, FLI1, C_8, In, 37, driver, PPDLHARHFLQLVWSGVTILDLPHGGDLPQPQRAPPP, (SEQ ID NO: 3510); XM_005621674.2, SRRM2, C_8, In, 38, essential, high expression, PSPRARGHVQRDRAADAPGQRHQRLRPAQPVPGAGPPG, (SEQ ID NO: 3511); XM_014111162.1, PADI2, C_8, Del, 38, high expression, PRTLLGAPCPTCPSVPHPMTRSLCLPTARPQLRPRPSA, (SEQ ID NO: 3512); XM_848403.4, STXS, C_8, In, 38, essential, PSGPGPSPSQHHVLPRSDPGVPVCLQVPAEPSEWNPDK, (SEQ ID NO: 3513); XM_855027.4, ATP2A1, C_8, In, 39, high expression, PEPQGAPHQWLALLPLHGNWGLCGCSHCGSCCLVVHVCR, (SEQ ID NO: 3514); XM_014122046.1, SMARCA4, C_8, Del, 40, driver, essential, PQDPQACPLGCQVSPPEGLPSLGLKDPWRMLLPPRAHLRS, (SEQ ID NO: 3515); XM_005626284.2, FLT4, C_8, Del, 40, driver, PLSVSSGTGGHGHPARPSPSAASAGGSRETTCHSARTGER, (SEQ ID NO: 3516); XM_005629711.2, NOM1, C_8, Del, 41, essential, PRGPGPPQPQPQPLPPLGNGLFWRPTRRRTGRSESWSGASA, (SEQ ID NO: 3517); XM_545182.4, ICE1, C_8, In, 41, essential, PATLARSFGRYTSKDFATSFSSDSFLSLLTYFSYWPDVPLM, (SEQ ID NO: 3518); XM_854451.4, ATXN2L, C_8, Del, 44, high expression, PKARCPRVGCLHSQLPHPHPIPTSDTPKFNLIPPSSSPSTPRGT, (SEQ ID NO: 3519); XM_854451.4, ATXN2L, C_8, In, 44, high expression, PRRGAPEWGACTLSFHTLTLSLHRTPPSSISSLPAAPLPPPGEL, (SEQ ID NO: 3520); XM_014110909.1, RAPH1, C_8, Del, 45, high expression, PLSCFLINRKLVTEVVIYRAMQRCGEDPLPLPPRETRIPSSQETG, (SEQ ID NO: 3521); XM_005619566.2, FLI1, C_8, Del, 45, driver, PTRPPCPSLPPACLERRHNTGPPPRGGSTPTPTCPATLTPTCPHT, (SEQ ID NO: 3522); XM_546606.6, PER1, C_8, Del, 45, driver, PHLPLLPSRLSSSPTLSRCSPLGEAPSLLPLPLQLGPLQPSLPRS, (SEQ ID NO: 3523); XM_846534.4, PRCC, C_8, Del, 47, driver, essential, PRKLPQMPPSSMTKHLSGCRARGTEGGRRSTLWRLKGMTSSVGPSSG, (SEQ ID NO: 3524); XM_005628850.2, YBX1, C_8, Del, 48, essential, high expression, PPPPPSAPPTPSPAPRAAAQGAAARAASHRRRLPAGTRRSSQRRFWEQ, (SEQ ID NO: 3525); XM_541488.5, PNKP, C_8, In, 48, essential, HLPALGRASPCPGPGTLDPGYRPEVLQKPSGAGRKPQDQDRGSETAGN, (SEQ ID NO: 3526); XM_855027.4, ATP2A1, C_8, Del, 49, high expression, PGAPRSPSSVAGSSSATWQLGAMWVQPLWELLPGGSCMQMTGLVSPTAS, (SEQ ID NO: 3527); XM_538528.5, BMP10, C_8, Del, 49, high expression, PDTKPTNAVGFATTPWQSTSHPQSTRSSRPWSTSRIPKRLPRSAVCPPS, (SEQ ID NO: 3528); XM_005621365.2, ATXN2L, C_8, Del, 50, high expression, PKARCPRVGCLHSQLPHPHPIPTSDTPKVSSLARRLDFQEEPMTGFVSSR, (SEQ ID NO: 3529); XM_014111339.1, KDM5C, C_8, Del, 52, driver, PKPCPLLAAPAPRRTRMAWSLRWPLQAPQPLSLLCLLSCMCPAHSSHLSNSC, (SEQ ID NO: 3530); XM_014111339.1, KDM5C, C_8, In, 52, driver, PNPAHYWQPQHPGEPEWLGACAGHFRPLSPFLYSVSSAACALPTAATSATVV, (SEQ ID NO: 3531); XM_005619036.2, SYNPO2L, C_8, In, 53, high expression, PTGAHPSAQPRASLRFPQHSTLLGPRHSHKFPVHPSPQSSCYARRSPRAPRPL, (SEQ ID NO: 3532); XM_847992.3, NCOR2, C_8, In, 53, driver, QQGLRAAAPGTPRLQSLGHRPHPSKEPRTPPRRPGPACAARLDHGPASGKDSK, (SEQ ID NO: 3533); XM_005639159.2, AFF1, C_8, Del, 54, driver, high expression, PHLCHPHPHPPRSQPSLHKRGQGGKLIPPAKTLPKVPVVARATTKTLPLPSTEK, (SEQ ID NO: 3534); XM_546606.6, PER1, C_8, In, 54, driver, PTCHYSLPGCRPALPSPGVLPSGRLPVSCLCPYSWAPCSLPCPARDPNGGLGAS, (SEQ ID NO: 3535); XM_005616609.2, HNRNPL, C_8, In, 56, essential, high expression, PTSLRREKDGPTSGGSPSGPKSLRPPVWAPPTPSPTTRVWPPRRQPCAHGLWLGSI, (SEQ ID NO: 3536); XM_014117151.1, SMARCC2, C_8, Del, 56, high expression, PEAWAPLNRLGRQGQPWGHSSSNQLEPPSLGQSHQGFPPLDPMAPHRSPTNKLLPQ, (SEQ ID NO: 3537); XM_005621674.2, SRRM2, C_8, Del, 57, essential, high expression, PLPPGTGPCTTGSGCRRPGAAAPTATSSATCPWCGAAGVSGLTTRERRNCGAWRLPW, (SEQ ID NO: 3538); XM_005616609.2, HNRNPL, C_8, Del, 58, essential, high expression, PHLTTKGEGWAHQWGVTVGAQVATAPSMGTPHPLPHHPSMAPTPTALCSWSMAWINLR, (SEQ ID NO: 3539); XM_005625495.2, SMARCC2, C_8, In, 58, high expression, PWTPWPLTVPQPTNSSRNDARGSARQRAPRRGGPRYPSASRPHGPEPRHSHPCATSTV, (SEQ ID NO: 3540); XM_005619036.2, SYNPO2L, C_8, Del, 58, high expression, PHRGPPLCPATCQPPVPPALHAPRAPSQPQVPCTSQPPIVLLRQEEPPSPPPPLALLP, (SEQ ID NO: 3541); XM_537509.5, YLPM1, C_8, In, 59, essential, high expression, PFSLLCSTSTCLAPPTIVFSHTSSGITSPWGSTRDTSSVSHSSSSTSLQFSKFTGSRET, (SEQ ID NO: 3542); XM_852361.4, TCF20, C_8, Del, 61, high expression, PRGAALAISMGARGMWASFKHSTLPLVVCLIISRITRGLSLQGVLSTNSRLPASSSSRYSS, (SEQ ID NO: 3543); XM_014118314.1, BAG6, C_8, Del, 61, essential, PQLPMRRQLPLVLGRPRPWLPLLPLSSLQPRGLPRQGQLPPQPPVTRGSSEFPTRAWNPWS, (SEQ ID NO: 3545); XM_005631865.2, WDR33, C_8, In, 62, essential, PGIARASRLTRSAGAAPGLFGASTPGWHARAPWTSGTAEPGKRATSISRANTIPAAENSSAR, (SEQ ID NO: 3546); XM_005628850.2, YBX1, C_8, In, 63, essential, high expression, RRPRPQRRRHQARHHGQRRRERRPGRPHIGGACRRGQEGHRNEGFGNSKMVQCKKRIWFHQQE, (SEQ ID NO: 3547); XM_005626284.2, FLT4, C_8, In, 63, driver, PSQCPVALAAMDTLQDLHQAQPQPAAAERPHATVPGLARGDHPGCREPHREPGHLDRVCGGEE, (SEQ ID NO: 3548); XM_014113873.1, PRDM16, C_8, Del, 64, driver, PQPSTTPAWASTSTSHPGHTPAACPSRRLHLPSPHSPRASRASSLHPCTPGRLCYLPRRCSRAP, (SEQ ID NO: 3549); XM_537035.5, RBM15, C_8, Del, 64, driver, PRLRHRCPVSWRESETIRSMRECARRTAWSPGWELEQVLLLSEKWMRSHPRTISELTGRFSWAT, (SEQ ID NO: 3550); XM_014115306.1, NES, C_8, Del, 65, high expression, PPCSSHRLGGSRFQRLYRLKPRWPSLPASCQDQRSLGASNERPVQASPLRMLPLWPHPSALITLV, (SEQ ID NO: 3551); XM_534998.5, PPRC1, C_8, Del, 65, essential, PHQQCPWYLVLLAPMLCPPLAMCLGYLLQPQSHLIAPAVPMGPWDGVQGCNTLHSGLLCHHLLCL, (SEQ ID NO: 3552); XM_014118314.1, BAG6, C_8, In, 65, essential, PNHQSPEGHPNFPPERGTRGHDAHEYPRFRHTAWWSSECSHWPPRTPWSWPDPGLHPHPAALPAP, (SEQ ID NO: 3553); XM_845621.5, MLLT6, C_8, Del, 67, driver, PLPQPLSPQRQTSLSRRWSSLALGPSCASPPPPPARPGPGPRPLETISLPTFRGLGPQQAPTSGCPH, (SEQ ID NO: 3555); XM_005623432.2, KTN1, C_8, Del, 68, driver, high expression, PLKQLPQRRNQDRRSLKMEAMTRIKRWKLLWHLQKSKNHYPSSKRLNKKVDQERRKFLQRSKRQ KMYW, (SEQ ID NO: 3556); XM_005631954.2, R3HDM1, C_8, Del, 68, high expression, PHPCPPGNQSLLLAILPQAILSASLCCSSRDIFSSPHHRCQPVTVLQATITPVNLSIAQSLLSITIHI, (SEQ ID NO: 3557); XM_848403.4, STXS, C_8, Del, 72, essential, PQWPWSLPLPTPCPAEIGPRSSCLPASPCRAVRMESRQISQLCVLSGSVANLPSWPSALGKTSATHLP SWRS, (SEQ ID NO: 3558); XM_005629711.2, NOM1, C_8, In, 74, essential, RGDQAPRSRSRSHCRRSETGSSGGQRGGGPGDPKAGAVPRLEQAQKEGRRQLRAAELCPRWARLH PRSPGVWKK, (SEQ ID NO: 3559); XM_005621364.2, ATXN2L, C_8, Del, 77, high expression, PKARCPRVGCLHSQLPHPHPIPTSDTPKYVGWAEATVAAARGLLLALSFASLRPRSVVIQQPPSPLPP QLSVTPTVS, (SEQ ID NO: 3560); XM_014117151.1, SMARCC2, C_8, In, 80, high expression, PWTPWPLTVPQPTNSSRNDARGSARQRAPRRGGPEPCHCGSCSGQPPAQCQPTARPRYPSASRPHG PEPRHSHPCATSTV, (SEQ ID NO: 3562); XM_847992.3, NCOR2, C_8, Del, 81, driver, PARAPSRGPWHPPAPVTRPSPAPQQRASHPTTPARTSLRRPPRPRTCIGKRLKVNPFSPRNWSSVLW VTMVAATAPTEWSP, (SEQ ID NO: 3563); XM_014120154.1, FAT1, C_8, In, 85, driver, high expression, PGARPSHLLHSKHSKRLQEQPGPQFLRRICHPRAPGIQHLQPRVWARTPQGRGRLQRGPEPASPAPF QLPFGQRLHPEAQLGLRL, (SEQ ID NO: 3565); XM_005626996.1, KLF4, G_8, In, 8, driver, high expression, GPFSSSPR, (SEQ ID NO: 3568); XM_538859.5, WDR46, G_8, In, 11, essential, GKFLEKGPPVK, (SEQ ID NO: 3569); XM_014114365.1, SETD1A, G_8, Del, 12, essential, GCPCPSRCKPRC, (SEQ ID NO: 3570); XM_005634924.1, ASXL1, G_8, In, 12, driver, GWPGWRWRRGHR, (SEQ ID NO: 3571); XM_005625443.2, FUBP3, G_8, In, 13, high expression, GQQSCPTPSSQGS, (SEQ ID NO: 3572); XM_532250.5, QRSL1, G_8, In, 13, essential, GGGGEGDRTLSTP, (SEQ ID NO: 3573); XM_847261.3, FOXA1, G_8, Del, 13, driver, GGSWILGRELNSS, (SEQ ID NO: 3574); XM_858955.4, GLYR1, G_8, In, 14, high expression, ALSGSPSLRESAAV, (SEQ ID NO: 3575); XM_859724.4, NRAP, G_8, Del, 14, high expression, GQRLWSPQRIRGGT, (SEQ ID NO: 3576); XM_005617051.2, SVIL, G_8, Del, 15, high expression, GRPPRMSGSLPSRRF, (SEQ ID NO: 3577); NM_001284453.1, PSMB7, G_8, In, 15, essential, GLQGWHSSWSRYKGN, (SEQ ID NO: 3578); XM_844466.4, MAZ, G_8, Del, 17, high expression, GPPPLSRETPPSHSPPV, (SEQ ID NO: 3579); XM_844466.4, MAZ, G_8, In, 17, high expression, GRPPCPGRRPPATAHLL, (SEQ ID NO: 3580); XM_005617051.2, SVIL, G_8, In, 17, high expression, VGHPECQEASHLEGSRK, (SEQ ID NO: 3581); XM_542166.4, ZBTB7A, G_8, Del, 19, essential, GAMGAPGPPPRAASRPDLP, (SEQ ID NO: 3582); XM_542166.4, ZBTB7A, G_8, In, 19, essential, GRWGRRGPRRGQLRGRTCL, (SEQ ID NO: 3583); XM_858955.4, GLYR1, G_8, Del, 20, high expression, GAFWKPQSQGISSCLMMGCW, (SEQ ID NO: 3584); XM_005628195.2, PDGFRA, G_8, Del, 22, driver, high expression, GAKACSGTESFPELWGLPFWHS, (SEQ ID NO: 3585); XM_014119028.1, NRF1, G_8, In, 22, essential, GTDRPVWGNRSSCRSTYWSPRC, (SEQ ID NO: 3586); XM_005619419.2, GOLPH3, G_8, Del, 24, high expression, GRGAGGGFHLHEDVARNPTALWSP, (SEQ ID NO: 3587); XM_005638688.1, MYOSA, G_8, Del, 24, driver, GGGTGRGRSPGPLVPGSSLPACNP, (SEQ ID NO: 3588); XM_536395.5, VCL, G_8, Del, 25, essential, high expression, GAKPNFALRRKSQNHIRVLLSLPCP, (SEQ ID NO: 3589); XM_014119359.1, CCDC9, G_8, Del, 27, high expression, GLTLSGCAAAWSRSGRAAGPAWAALAT, (SEQ ID NO: 3590); XM_546016.5, ELL2, G_8, Del, 27, high expression, GRRACGRSSATGCRAGGWDRTTSRCCM, (SEQ ID NO: 3591); XM_005626996.1, KLF4, G_8, Del, 29, driver, high expression, GAVLFLSQVRNPTTVIGTAVGGNSPARTN, (SEQ ID NO: 3593); XM_014119037.1, NRF1, G_8, Del, 29, essential, GDRSSCLGKPQQLSEHLLESKMLMVPTSF, (SEQ ID NO: 3594); XM_536395.5, VCL, G_8, In, 30, essential, high expression, VQNQILLSGGNRRITFVCFCLYLAPEQRLC, (SEQ ID NO: 3595); XM_014119028.1, NRF1, G_8, Del, 30, essential, GDRSSCLGKPQQLSEHLLESKMLMDWSRSL, (SEQ ID NO: 3596); XM_014114200.1, SRRT, G_8, In, 32, essential, high expression, GWGPYLWPPSALGPPRRPHHAAPCSAYPGQTS, (SEQ ID NO: 3597); XM_538859.5, WDR46, G_8, Del, 32, essential, GEISGKRPPGKMTKSGRALSNSHRSKRRKPSP, (SEQ ID NO: 3598); XM_859724.4, NRAP, G_8, In, 33, high expression, ARGCGARRESEGGREPRRHRDSARQKEEGSAVV, (SEQ ID NO: 3599); XM_014111383.1, RBM10, G_8, In, 35, driver, essential, AGAQAQPHWPPRFPPRRGLSGPGLSAPPMGGSGPF, (SEQ ID NO: 3600); XM_847680.4, CHERP, G_8, Del, 35, essential, GTSETSGTSTRVWAWPWTIHTRTTAGTRATPSSPA, (SEQ ID NO: 3601); XM_542234.5, MAML2, G_8, Del, 36, driver, GPPGRGSLEGARSPRGCTAPSWSACGLGLPSAASTT, (SEQ ID NO: 3602); XM_003434769.3, SRRT, G_8, In, 37, essential, high expression, GWGPYLWPPSALGPPRRPHHAAPCSAYPGQAGQHRRD, (SEQ ID NO: 3603); XM_542234.5, MAML2, G_8, In, 42, driver, GPRGGAPWRGLGHPEGAQRHRGAPAGSDCHLPPAPPELRRPL, (SEQ ID NO: 3604); XM_847680.4, CHERP, G_8, In, 43, essential, GRPRQVGPVQGCGRGPGRSIRELPPEQELLLHRPHEGQGRVQV, (SEQ ID NO: 3605); XM_003434769.3, SRRT, G_8, Del, 45, essential, high expression, GVGALLMAPLSPGATQTSTSCSTMFCLSRPGWAASQRLTWVCHHR, (SEQ ID NO: 3606); XM_005628195.2, PDGFRA, G_8, In, 46, driver, high expression, EPKPARGPKVFRNYGDFSPGTPDLRLSPRRAQPNPLPAFITLDPSK, (SEQ ID NO: 3607); XM_003638990.3, SYNPO2L, G_8, Del, 46, high expression, GPSRGNLYRCPRSEDGARLAEQDSERGTSFWPSMGSPAPASPMPVP, (SEQ ID NO: 3608); XM_548255.6, TAF15, G_8, In, 48, driver, high expression, GLWRRPRWWRGLRWRPRWRLRRRQKWGRLWRRPRRLWRQNGRKKRLQK, (SEQ ID NO: 3609); XM_014111383.1, RBM10, G_8, Del, 50, driver, essential, GGGTGTAPLAPQVSPETGTFGTGTFGPTNGRIWPFLRRRRRRRSPVTSSC, (SEQ ID NO: 3611); XM_014114200.1, SRRT, G_8, Del, 53, essential, high expression, GVGALLMAPLSPGATQTSTSCSTMFCLSRPDFLTGPPPGWAASQRLTWVCHHR, (SEQ ID NO: 3613); XM_548255.6, TAF15, G_8, Del, 56, driver, high expression, GAMEETAVVAGATVETEVAATEETEVGAAMEETEEAMEAKWEEETTTEMISATDHT, (SEQ ID NO: 3614); XM_005629758.2, PSD3, G_8, Del, 58, high expression, GEMMISLGMGMQRTPLMCTAPSLKLFWTTLRYTTVLNPWRHYIQNPIAILALTCPSLR, (SEQ ID NO: 3615); XM_005634924.1, ASXL1, G_8, Del, 58, driver, GVARVEVAAGPPMREVAEAAAVVMVVRPVATLSPVEPRTPLESVRQIYSEHNYCRLVL, (SEQ ID NO: 3616); XM_005638688.1, MYOSA, G_8, In, 60, driver, AGARAGVGPLVPWSPAVRYLRATPRALFGSMPPASAGRASCFRIAFEGSVLSLLYRRQYF, (SEQ ID NO: 3617); XM_003638990.3, SYNPO2L, G_8, In, 65, high expression, GRAEETFTGVQDPKTEPGWQSRTPREGPAFGHQWGLLHQPLPCQCHEPHRCLRESAYPHSAAGRG, (SEQ ID NO: 3619); XM_005626238.1, EPCAM, G_8, In, 69, high expression, GGAGAEGPPRGVGGRPPPPHLLPPGAGRWPRMCASRGHRWLRGGRAARTCAGAGPGPQRLARCP LVMYL, (SEQ ID NO: 3620); XM_534311.5, DHX36, A_7, Del, 8, essential, high expression, KLLLVFGK, (SEQ ID NO: 3625); XM_005626023.2, EIF5B, A_7, Del, 8, essential, high expression, KDKKSVSK, (SEQ ID NO: 3626); XM_014121388.1, IWS1, A_7, In, 8, essential, high expression, KHEWQAQA, (SEQ ID NO: 3627); XM_545261.4, NMD3, A_7, Del, 8, essential, high expression, KLSTIWNS, (SEQ ID NO: 3628); XM_005621408.2, RBBP6, A_7, In, 8, essential, high expression, NQAQQRNW, (SEQ ID NO: 3629); XM_005621408.2, RBBP6, A_7, In, 8, essential, high expression, NWKYRKCI, (SEQ ID NO: 3630); XM_534762.5, MRPL40, A_7, In, 8, essential, high expression, KDQTTGES, (SEQ ID NO: 3631); XM_005620768.2, NFAT5, A_7, In, 8, essential, high expression, KHSCQIGF, (SEQ ID NO: 3632); XM_014107297.1, PSMA7, A_7, In, 8, essential, high expression, KETKESIM, (SEQ ID NO: 3633); XM_014107297.1, PSMA7, A_7, Del, 8, essential, high expression, KRNKRKHH, (SEQ ID NO: 3634); NM_001171748.1, RBM12, A_7, In, 8, essential, high expression, RVKDTCAR, (SEQ ID NO: 3635); XM_544630.4, RTF1, A_7, Del, 8, essential, high expression, KDLKSGKN, (SEQ ID NO: 3636); XM_535608.5, SDAD1, A_7, In, 8, essential, high expression, KEEETRGV, (SEQ ID NO: 3637); XM_014116842.1, SMG6, A_7, Del, 8, essential, high expression, KSNMRNWN, (SEQ ID NO: 3638); XM_005618133.2, MEF2C, A_7, Del, 8, essential, high expression, KLMKNLII, (SEQ ID NO: 3639); XM_014109172.1, PRKDC, A_7, Del, 8, essential, high expression, KEDHGFKK, (SEQ ID NO: 3640); XM_014118978.1, AKAP9, A_7, Del, 8, driver, high expression, KCMSSSPK, (SEQ ID NO: 3641); NM_001287021.1, IL6ST, A_7, Del, 8, driver, high expression, KWGKMKLS, (SEQ ID NO: 3642); XM_850315.4, IL7R, A_7, Del, 8, driver, high expression, KMKMTGRV, (SEQ ID NO: 3643); XM_014113182.1, JAK1, A_7, In, 8, driver, high expression, NGTEALIP, (SEQ ID NO: 3644); XM_003638878.3, KIF5B, A_7, Del, 8, driver, high expression, KWKKMKRS, (SEQ ID NO: 3645); XM_005624991.1, MED13, A_7, In, 8, high expression, TQGRRWAI, (SEQ ID NO: 3646); XM_014106380.1, AKAP11, A_7, Del, 8, high expression, KINLKIHP, (SEQ ID NO: 3647); XM_005616994.2, ARHGAP21, A_7, Del, 8, high expression, KIRWNQET, (SEQ ID NO: 3648); XM_536845.5, BAZ1B, A_7, Del, 8, high expression, NLRQTTLL, (SEQ ID NO: 3649); XM_014119556.1, EEA1, A_7, Del, 8, high expression, KTTTYRNK, (SEQ ID NO: 3650); XM_014106344.1, PHF11, A_7, Del, 8, high expression, KKSTEGGS, (SEQ ID NO: 3651); XM_005627518.2, PHF3, A_7, Del, 8, high expression, KLLQQSMN, (SEQ ID NO: 3652); XM_014108414.1, PLEKHA5, A_7, In, 8, high expression, SSQFWKEI, (SEQ ID NO: 3653); XM_005623023.2, ROCK1, A_7, In, 8, high expression, SKSEFTAC, (SEQ ID NO: 3654); XM_005633448.2, RSF1, A_7, Del, 8, high expression, KEEKEKMR, (SEQ ID NO: 3655); XM_005618779.2, RYR2, A_7, Del, 8, high expression, KTAPCRLY, (SEQ ID NO: 3656); XM_005638661.2, SCAPER, A_7, In, 8, high expression, TPASCERE, (SEQ ID NO: 3657); XM_005623486.2, SYNE2, A_7, In, 8, high expression, KCLARSLF, (SEQ ID NO: 3658); XM_531794.5, TMEM131, A_7, In, 8, high expression, KYTIQNHC, (SEQ ID NO: 3659); XM_014119604.1, TMPO, A_7, Del, 8, high expression, KDAPFPCG, (SEQ ID NO: 3660); XM_014107674.1, TRAF3IP1, A_7, In, 8, high expression, NSGDQERL, (SEQ ID NO: 3661); XM_544645.5, UBR1, A_7, In, 8, high expression, KEKTRKQR, (SEQ ID NO: 3662); XM_005618617.2, UGDH, A_7, In, 8, high expression, NAEASLYL, (SEQ ID NO: 3663); XM_014117473.1, VPS13A, A_7, In, 8, high expression, NAFKTIEE, (SEQ ID NO: 3664); XM_005638476.2, VPS13C, A_7, In, 8, high expression, TQAVAQEL, (SEQ ID NO: 3665); XM_014106549.1, ZBTB38, A_7, In, 8, high expression, NSKWRPEA, (SEQ ID NO: 3666); XM_014118132.1, ZNF484, A_7, In, 8, high expression, NPYWRETL, (SEQ ID NO: 3667); XM_005630575.2, ZNF638, A_7, In, 8, high expression, RYRRFQRC, (SEQ ID NO: 3668); XM_014114846.1, ZNF644, A_7, Del, 8, high expression, KRESEKWM, (SEQ ID NO: 3669); XM_003434574.3, ATP5L, A_7, In, 8, high expression, NSQECSNW, (SEQ ID NO: 3670); XM_014110756.1, BAZ2B, A_7, Del, 8, high expression, KSGQIFSK, (SEQ ID NO: 3671); XM_014112204.1, CAST, A_7, Del, 8, high expression, KERWRRTQ, (SEQ ID NO: 3672); XM_005629294.2, CCDC53, A_7, In, 8, high expression, KYRRKFRQ, (SEQ ID NO: 3673); XM_005639670.2, CMBL, A_7, In, 8, high expression, TLQSGISN, (SEQ ID NO: 3674); XM_534301.5, CP, A_7, Del, 8, high expression, KILTKSLW, (SEQ ID NO: 3675); NM_001033996.2, CTSK, A_7, In, 8, high expression, KLEAYFYP, (SEQ ID NO: 3676); XM_005639750.2, FXR1, A_7, Del, 8, high expression, KKLVKEMK, (SEQ ID NO: 3677); XM_014115649.1, GALC, A_7, In, 8, high expression, MVYAHVNY, (SEQ ID NO: 3678); XM_014122251.1, IP6K2, A_7, Del, 8, high expression, NLKSCTTV, (SEQ ID NO: 3679); XM_533539.4, KIAA0020, A_7, Del, 8, high expression, KTKTPAKE, (SEQ ID NO: 3680); XM_533351.4, KIF5C, A_7, Del, 8, high expression, KSSVGSFI, (SEQ ID NO: 3681); XM_014120511.1, KRCC1, A_7, In, 8, high expression, TRGGTAQA, (SEQ ID NO: 3682); XM_005619490.2, LEPR, A_7, In, 8, high expression, KREWCVFD, (SEQ ID NO: 3683); XM_846278.4, MAP4K5, A_7, Del, 8, high expression, KDQLLKDF, (SEQ ID NO: 3684); XM_846278.4, MAP4K5, A_7, In, 8, high expression, KWWLQPAL, (SEQ ID NO: 3685); XM_014112389.1, MEF2A, A_7, Del, 8, high expression, KLMRNLII, (SEQ ID NO: 3686); XM_014106295.1, MYCBP2, A_7, Del, 8, high expression, KHLYNKSK, (SEQ ID NO: 3687); XM_005626304.2, RUFY1, A_7, Del, 8, high expression, KTTKLCPA, (SEQ ID NO: 3688); XM_545669.5, SCG2, A_7, Del, 8, high expression, KMNRSTTK, (SEQ ID NO: 3689); XM_005623792.2, SETD3, A_7, In, 8, high expression, TKRSVCYF, (SEQ ID NO: 3690); XM_005638171.2, UACA, A_7, In, 8, high expression, NWRDRKRV, (SEQ ID NO: 3691); XM_003433617.3, USMG5, A_7, In, 8, high expression, NTSCESNI, (SEQ ID NO: 3692); XM_005638799.2, USP16, A_7, In, 8, high expression, KFEKDNGG, (SEQ ID NO: 3693); XM_005633308.2, ZBED5, A_7, In, 8, high expression, NRCSTTIK, (SEQ ID NO: 3694); XM_014107325.1, BRCA2, A_7, Del, 8, driver, essential, KTNKTPKT, (SEQ ID NO: 3695); XM_005628719.2, ANLN, A_7, Del, 8, essential, NAVLTTLK, (SEQ ID NO: 3696); XM_014112349.1, BOD1L1, A_7, In, 8, essential, KKERFKEC, (SEQ ID NO: 3697); XM_014112349.1, BOD1L1, A_7, Del, 8, essential, KMILKPHI, (SEQ ID NO: 3698); XM_539598.4, CLSPN, A_7, Del, 8, essential, KRWKKSGG, (SEQ ID NO: 3699); XM_542610.5, DIS3, A_7, Del, 8, essential, KEESKKGL, (SEQ ID NO: 3700); XM_005630977.2, DNAJC2, A_7, Del, 8, essential, KVSLNLVI, (SEQ ID NO: 3701); XM_543400.5, NAA25, A_7, Del, 8, essential, KDWRPQRN, (SEQ ID NO: 3702); XM_005630719.2, POLR3C, A_7, In, 8, essential, SPLCAYPT, (SEQ ID NO: 3703); XM_849077.4, PPAT, A_7, In, 8, essential, IWSIVGQL, (SEQ ID NO: 3704); XM_014120363.1, PTCD3, A_7, Del, 8, essential, KTPIPTAQ, (SEQ ID NO: 3705); XM_539419.4, RBM48, A_7, Del, 8, essential, NYRRGRLI, (SEQ ID NO: 3706); XM_535878.4, RIOK1, A_7, Del, 8, essential, KERRRQPG, (SEQ ID NO: 3707); XM_844234.4, TAF1, A_7, In, 8, essential, SLSFSLRK, (SEQ ID NO: 3708); XM_003432210.3, TAF1B, A_7, Del, 8, essential, KRLKLANS, (SEQ ID NO: 3709); XM_540029.5, TRAPPC11, A_7, In, 8, essential, NRKCRAVF, (SEQ ID NO: 3710); XM_005639425.2, TRMT10C, A_7, Del, 8, essential, KLGKQKRN, (SEQ ID NO: 3711); XM_534297.5, U2SURP, A_7, In, 8, essential, KSSKSVIQ, (SEQ ID NO: 3712); XM_014111598.1, MBTPS2, A_7, Del, 8, essential, KVQVQVSV, (SEQ ID NO: 3713); XM_003639701.3, NIFK, A_7, Del, 8, essential, KDKEKAIS, (SEQ ID NO: 3714); XM_003639701.3, NIFK, A_7, In, 8, essential, KTKKKQSV, (SEQ ID NO: 3715); XM_014120448.1, EML4, A_7, In, 8, driver, KQPRRRIC, (SEQ ID NO: 3716); XM_014116492.1, NF1, A_7, In, 8, driver, KGKQKISG, (SEQ ID NO: 3717); XM_005619838.2, CWF19L2, A_7, In, 9, essential, high expression, SKEREEKKE, (SEQ ID NO: 3718); XM_014116402.1, DDX42, A_7, Del, 9, essential, high expression, KIFTMSMKR, (SEQ ID NO: 3719); XM_845208.4, HECTD1, A_7, In, 9, essential, high expression, NHNKNFAAD, (SEQ ID NO: 3720); XM_014120239.1, SOS1, A_7, In, 9, essential, high expression, NCKRQWTRS, (SEQ ID NO: 3721); XM_005625616.2, CCT2, A_7, In, 9, essential, high expression, NWSKSTKAN, (SEQ ID NO: 3722); XM_005630347.2, CEBPZ, A_7, In, 9, essential, high expression, IRVCFLGAL, (SEQ ID NO: 3723); XM_537353.5, CPSF2, A_7, Del, 9, essential, high expression, NLSSQKRQT, (SEQ ID NO: 3724); XM_005632455.2, GNL3, A_7, Del, 9, essential, high expression, KRFESTIEN, (SEQ ID NO: 3725); XM_005623593.2, MED6, A_7, Del, 9, essential, high expression, KRQNLYQKR, (SEQ ID NO: 3726); NM_001286231.1, TAF9, A_7, Del, 9, essential, high expression, KRHQLLREE, (SEQ ID NO: 3727); XM_532552.5, UTP11L, A_7, Del, 9, essential, high expression, KIQMNSTIK, (SEQ ID NO: 3728); XM_532920.5, WDR43, In, 9, essential, high expression, KEIRSYRNE, (SEQ ID NO: 3729); XM_014109172.1, PRKDC, A_7, Del, 9, essential, high expression, KSWAFQGTK, (SEQ ID NO: 3730); XM_014118978.1, AKAP9, A_7, In, 9, driver, high expression, KERRLQNAN, (SEQ ID NO: 3731); XM_538283.5, PTPRB, A_7, Del, 9, driver, high expression, KLAWEQKFQ, (SEQ ID NO: 3732); XM_846524.3, ACSL3, A_7, Del, 9, driver, high expression, KEAKGIQLC, (SEQ ID NO: 3733); XM_005632452.1, PBRM1, A_7, In, 9, driver, high expression, NILYEKGRN, (SEQ ID NO: 3734); XM_014120373.1, ALMS1, A_7, In, 9, high expression, KEWVTIRSF, (SEQ ID NO: 3735); XM_014115338.1, ASH1L, A_7, Del, 9, high expression, KLQRETMDN, (SEQ ID NO: 3736); XM_854584.4, ATF4, A_7, In, 9, high expression, NGAEQDSSH, (SEQ ID NO: 3737); XM_544889.6, BRWD1, A_7, In, 9, high expression, KEHQSNQLC, (SEQ ID NO: 3738); XM_537686.5, COIL, A_7, In, 9, high expression, KQEEKQSHV, (SEQ ID NO: 3739); XM_005618961.2, DDX50, A_7, In, 9, high expression, IHHQIRTG, (SEQ ID NO: 3740); XM_544733.5, DENND4A, A_7, In, 9, high expression, NAVEKDGST, (SEQ ID NO: 3741); XM_005638410.2, DMXL2, A_7, Del, 9, high expression, KTLLIKLIL, (SEQ ID NO: 3742); XM_014113123.1, FYB, A_7, Del, 9, high expression, KGKRRKRRD, (SEQ ID NO: 3743); XM_531770.5, GCC2, A_7, Del, 9, high expression, KTPKSSSCL, (SEQ ID NO: 3744); XM_005641869.2, MAP7D3, A_7, In, 9, high expression, KGKHQTDTL, (SEQ ID NO: 3745); XM_005641869.2, MAP7D3, A_7, Del, 9, high expression, KGKTSDRHL, (SEQ ID NO: 3746); XM_005641869.2, MAP7D3, A_7, Del, 9, high expression, KQKRREARL, (SEQ ID NO: 3747); XM_005629056.2, NASP, A_7, In, 9, high expression, TRRPVSGKA, (SEQ ID NO: 3748); XM_005633448.2, RSF1, A_7, Del, 9, high expression, KQRRIRILK, (SEQ ID NO: 3749); XM_005627265.2, SRPK1, A_7, In, 9, high expression, NYPASVTGS, (SEQ ID NO: 3750); XM_014112808.1, SYNE1, A_7, Del, 9, high expression, KSTVVISSW, (SEQ ID NO: 3751); XM_005623486.2, SYNE2, A_7, Del, 9, high expression, KSPFWISCQ, (SEQ ID NO: 3752); XM_005615794.2, ZCCHC6, A_7, Del, 9, high expression, KMRQIALWV, (SEQ ID NO: 3753); XM_534518.5, POMP, A_7, Del, 9, high expression, KISSSTKIK, (SEQ ID NO: 3754); XM_014112733.1, ARID4B, A_7, In, 9, high expression, NTEGAFFRL, (SEQ ID NO: 3755); XM_014110756.1, BAZ2B, A_7, In, 9, high expression, KKNGRKHFC, (SEQ ID NO: 3756); XM_005619370.2, C9, A_7, In, 9, high expression, NCPREDIKF, (SEQ ID NO: 3757); XM_014112203.1, CAST, A_7, Del, 9, high expression, KERWRSRTQ, (SEQ ID NO: 3758); XM_014112204.1, CAST, A_7, In, 9, high expression, KKGGGGRNE, (SEQ ID NO: 3759); XM_005634496.2, CEP70, A_7, In, 9, high expression, KGRGPRETR, (SEQ ID NO: 3760); XM_547771.4, CFL2, A_7, Del, 9, high expression, KERKQFSSV, (SEQ ID NO: 3761); XM_547299.4, CLCA1, A_7, In, 9, high expression, RSSKMFSRY, (SEQ ID NO: 3762); XM_539002.5, COL12A1, A_7, Del, 9, high expression, KFHTKVATQ, (SEQ ID NO: 3763); XM_848166.4, FAM135A, A_7, Del, 9, high expression, KRLKKPLKK, (SEQ ID NO: 3764); XM_533351.4, KIF5C, A_7, In, 9, high expression, KAQWEALSS, (SEQ ID NO: 3765); XM_014117435.1, MAP4K4, A_7, Del, 9, high expression, NGQRNFLVL, (SEQ ID NO: 3766); XM_535817.5, MCCC1, A_7, In, 9, high expression, NGYTVRSCV, (SEQ ID NO: 3767); XM_005635519.2, MTIF3, A_7, In, 9, high expression, REECRGART, (SEQ ID NO: 3768); XM_014106330.1, PIBF1, A_7, In, 9, high expression, SEHLQFSGI, (SEQ ID NO: 3769); XM_534366.4, PTPRA, A_7, In, 9, high expression, KPICKHLAL, (SEQ ID NO: 3770); XM_535303.4, RBL2, A_7, Del, 9, high expression, KSGPVLNSP, (SEQ ID NO: 3771); XM_014118822.1, REST, A_7, In, 9, high expression, KYKEEKSEK, (SEQ ID NO: 3772); XM_014108806.1, SORBS1, A_7, Del, 9, high expression, KISTSAQQS, (SEQ ID NO: 3773); XM_535814.5, TTC14, A_7, In, 9, high expression, KYRGKKRAP, (SEQ ID NO: 3774); XM_003434394.3, UBE2K, A_7, In, 9, high expression, NRKPMCYGL, (SEQ ID NO: 3775); XM_014112442.1, BLM, A_7, Del, 9, driver, essential, NPRRWHLIA, (SEQ ID NO: 3776); XM_014107325.1, BRCA2, A_7, In, 9, driver, essential, KQTKLQRHR, (SEQ ID NO: 3777); XM_003639986.2, CEP55, A_7, Del, 9, essential, KISRLNDRP, (SEQ ID NO: 3778); XM_003433767.3, DIMT1, A_7, In, 9, essential, KLQNPLFAP, (SEQ ID NO: 3779); XM_546165.5, DNAJC9, A_7, Del, 9, essential, KRLSRKKRN, (SEQ ID NO: 3780); XM_546165.5, DNAJC9, A_7, In, 9, essential, NASQERKEI, (SEQ ID NO: 3781); XM_536459.3, GEMIN5, A_7, Del, 9, essential, KISHFLCLN, (SEQ ID NO: 3782); XM_014115881.1, MIS18BP1, A_7, In, 9, essential, NNRTEMHAV, (SEQ ID NO: 3783); XM_014119696.1, MRPL42, A_7, Del, 9, essential, KMNTLSKDS, (SEQ ID NO: 3784); XM_014108253.1, MRPS35, A_7, Del, 9, essential, KISYKHFSK, (SEQ ID NO: 3785); XM_005631968.2, ORC4, A_7, Del, 9, essential, KVTGLVAAQ, (SEQ ID NO: 3786); XM_535884.5, PAK1IP1, A_7, In, 9, essential, KEEKKEENI, (SEQ ID NO: 3787); XM_535884.5, PAK1IP1, A_7, Del, 9, essential, KRREKRRKH, (SEQ ID NO: 3788); XM_014118218.1, REV3L, A_7, Del, 9, essential, KTICHLSES, (SEQ ID NO: 3789); XM_005636930.1, RPAP3, A_7, Del, 9, essential, KLHMYCSIT, (SEQ ID NO: 3790); XM_014117890.1, SMC2, A_7, Del, 9, essential, KPLRKVRRP, (SEQ ID NO: 3791); XM_532882.5, SMC6, In, 9, essential, IGVNSGQRN, (SEQ ID NO: 3792); XM_014115128.1, SMG7, A_7, In, 9, essential, NEAFSHGAI, (SEQ ID NO: 3793); XM_005624831.2, TEFM, A_7, Del, 9, essential, KESSRTTGS, (SEQ ID NO: 3794); XM_005627564.1, TTK, A_7, Del, 9, essential, KVNNFFIKL, (SEQ ID NO: 3795); XM_535769.5, UMPS, A_7, Del, 9, essential, KLMLRWSRE, (SEQ ID NO: 3796); XM_535769.5, UMPS, A_7, Del, 9, essential, NSMKVESSK, (SEQ ID NO: 3797); XM_014119477.1, UTP20, A_7, Del, 9, essential, KLKRNSQYP, (SEQ ID NO: 3798); XM_005620578.2, ZC3H18, A_7, In, 9, essential, SNHSKRTGA, (SEQ ID NO: 3799); XM_845305.4, CCNB1, A_7, In, 9, essential, NTKASGEGT, (SEQ ID NO: 3800); XM_532318.5, TAF2, A_7, In, 9, essential, NSIDEWRRS, (SEQ ID NO: 3801); XM_005615937.2, CD274, A_7, Del, 9, driver, NMIYNLKRH, (SEQ ID NO: 3802); NM_001002963.1, MET, A_7, In, 9, driver, NSLCCEIPE, (SEQ ID NO: 3803); XM_005629880.1, NRG1, A_7, In, 9, driver, KAREVRTSH, (SEQ ID NO: 3804); XM_847054.5, TRIM24, A_7, Del, 9, driver, KRKLKALLN, (SEQ ID NO: 3805); XM_005641516.2, ATRX, A_7, Del, 10, driver, essential, high expression, KEWIIKGRKI, (SEQ ID NO: 3806); XM_014116355.1, BPTF, A_7, Del, 10, essential, high expression, KTTWKQNHVY, (SEQ ID NO: 3807); XM_532156.6, CDC5L, A_7, In, 10, essential, high expression, KACTWFLRYF, (SEQ ID NO: 3808); XM_534311.5, DHX36, A_7, In, 10, essential, high expression, KKNGQGLHKN, (SEQ ID NO: 3809); XM_005626022.2, EIF5B, A_7, Del, 10, essential, high expression, KGKTLMKMIS, (SEQ ID NO: 3810); XM_005634774.2, ESF1, A_7, In, 10, essential, high expression, KTEEETEGSC, (SEQ ID NO: 3811); XM_003640020.3, MRPL15, A_7, In, 10, essential, high expression, KWWCCYYSFL, (SEQ ID NO: 3812); XM_005630347.2, CEBPZ, A_7, In, 10, essential, high expression, RKCQQKRSNI, (SEQ ID NO: 3813); XM_005630473.2, IMMT, A_7, Del, 10, essential, high expression, KKRFLGPNLI, (SEQ ID NO: 3814); XM_003639075.3, NAE1, A_7, Del, 10, essential, high expression, KRIIAILHGL, (SEQ ID NO: 3815); XM_005628224.2, PAICS, A_7, Del, 10, essential, high expression, KIYENNFMEH, (SEQ ID NO: 3816); XM_014109172.1, PRKDC, A_7, In, 10, essential, high expression, KAGPSRGQSR, (SEQ ID NO: 3817); XM_014113182.1, JAK1, A_7, In, 10, driver, high expression, KQTETEKTGK, (SEQ ID NO: 3818); XM_005623935.2, HSP90AA1, A_7, Del, 10, driver, high expression, KQSLKICARS, (SEQ ID NO: 3819); XM_014108062.1, DENR, A_7, Del, 10, high expression, KRRLYHKKLR, (SEQ ID NO: 3820); XM_014111218.1, ZC3H11A, A_7, In, 10, high expression, NSCGSCSTPF, (SEQ ID NO: 3821); XM_005615442.2, AHI1, A_7, In, 10, high expression, NRQIWRKNCT, (SEQ ID NO: 3822); XM_532457.5, CYP51A1, A_7, Del, 10, high expression, KICLKLFRSS, (SEQ ID NO: 3823); XM_005617306.2, DIAPH1, A_7, In, 10, high expression, KAGLRVNSPT, (SEQ ID NO: 3824); XM_014106605.1, GOLGA4, A_7, Del, 10, high expression, NISKRSLMHL, (SEQ ID NO: 3825); XM_005637042.2, ITPR2, A_7, In, 10, high expression, KVRKILQSSL, (SEQ ID NO: 3826); XM_014109639.1, LONP2, A_7, Del, 10, high expression, KCLSQCQNML, (SEQ ID NO: 3827); XM_005628918.2, MACF1, A_7, In, 10, high expression, NAKFRIKTRV, (SEQ ID NO: 3828); XM_014121880.1, MAP4, A_7, In, 10, high expression, NRGICSGSKA, (SEQ ID NO: 3829); XM_005641869.2, MAP7D3, A_7, In, 10, high expression, NRKEEKLVFD, (SEQ ID NO: 3830); XM_014111164.1, MIA3, A_7, Del, 10, high expression, NMFKKPRNKI, (SEQ ID NO: 3831); XM_544755.5, MYO9A, A_7, Del, 10, high expression, KIQIICALTL, (SEQ ID NO: 3832); XM_014119574.1, OSBPL8, A_7, Del, 10, high expression, KMVSGLEQFF, (SEQ ID NO: 3833); XM_005635475.2, PDS5B, A_7, Del, 10, high expression, KHLSQIQKRN, (SEQ ID NO: 3834); XM_860876.4, SPAG9, A_7, Del, 10, high expression, KRIYRPEWNL, (SEQ ID NO: 3835); XM_855384.3, USP15, A_7, Del, 10, high expression, KDCLHSSSTI, (SEQ ID NO: 3836); XM_014117467.1, VPS13A, A_7, Del, 10, high expression, KMRLNFFIKI, (SEQ ID NO: 3837); XM_855243.4, BCLAF1, A_7, Del, 10, high expression, KIINLKRLAT, (SEQ ID NO: 3838); XM_005621312.2, AGL, A_7, Del, 10, high expression, KEFSLIMKSE, (SEQ ID NO: 3839); XM_014110756.1, BAZ2B, A_7, In, 10, high expression, KVDRYFQSEL, (SEQ ID NO: 3840); XM_003434950.3, C4BPA, A_7, Del, 10, high expression, NSKCLVLERH, (SEQ ID NO: 3841); XM_014120778.1, CACNA2D1, A_7, Del, 10, high expression, KMRVSQAARG, (SEQ ID NO: 3842); XM_014112203.1, CAST, A_7, In, 10, high expression, KKGGGAGRNE, (SEQ ID NO: 3843); XM_535304.5, CHD9, A_7, In, 10, high expression, IRRRCRRETI, (SEQ ID NO: 3844); XM_005615711.2, HDDC2, A_7, In, 10, high expression, KVSQLGHVFP, (SEQ ID NO: 3845); XM_005636851.2, LIMA1, A_7, In, 10, high expression, NGNVCRRTQA, (SEQ ID NO: 3846); XM_005622961.2, PRG4, A_7, Del, 10, high expression, NLTCSPGPLC, (SEQ ID NO: 3847); XM_005622961.2, PRG4, A_7, Del, 10, high expression, NQRLHQLPQR, (SEQ ID NO: 3848); XM_542038.5, RAD23A, A_7, Del, 10, high expression, KTRTWLPTSS, (SEQ ID NO: 3849); XM_845578.4, PALB2, A_7, Del, 10, driver, essential, KLRSLKITQV, (SEQ ID NO: 3850); XM_014116764.1, ATAD5, A_7, Del, 10, essential, KRNKRKRWKF, (SEQ ID NO: 3851); XM_014107179.1, AURKA, A_7, Del, 10, essential, KTKTQLANSL, (SEQ ID NO: 3852); XM_005630977.2, DNAJC2, A_7, In, 10, essential, RRDEQNKNIS, (SEQ ID NO: 3853); XM_536013.5, GTF3C3, A_7, In, 10, essential, KCRRRNFRRE, (SEQ ID NO: 3854); XM_014122593.1, MCM10, A_7, In, 10, essential, KERTTYLPGI, (SEQ ID NO: 3855); XM_014116428.1, NOL11, A_7, Del, 10, essential, KIREWLDHCC, (SEQ ID NO: 3856); XM_014118218.1, REV3L, A_7, In, 10, essential, KLFVIYRKVK, (SEQ ID NO: 3857); XM_005636930.1, RPAP3, A_7, Del, 10, essential, KPKMKTQKTG, (SEQ ID NO: 3858); XM_005633939.2, SUGT1, A_7, Del, 10, essential, KMRSWREMQL, (SEQ ID NO: 3859); XM_005626867.1, TEX10, A_7, Del, 10, essential, KENAKMIFKK, (SEQ ID NO: 3860); XM_005634448.1, TOPBP1, A_7, In, 10, essential, NPAKTRIRHC, (SEQ ID NO: 3861); XM_534297.5, U2SURP, A_7, Del, 10, essential, KAIWNSSKKN, (SEQ ID NO: 3862); XM_014119418.1, CHD8, A_7, In, 10, essential, KPSTQTGNYH, (SEQ ID NO: 3863); XM_014118286.1, DHX16, A_7, In, 10, essential, NRQDTGRARI, (SEQ ID NO: 3864); XM_549075.3, ERCC6L, A_7, In, 10, essential, KVKYPRGDQI, (SEQ ID NO: 3865); XM_005615806.2, NAA35, A_7, Del, 10, essential, KQRKRRKFAH, (SEQ ID NO: 3866); XM_005615620.2, RSPO3, A_7, In, 10, driver, RKGEEKEKTQ, (SEQ ID NO: 3867); XM_005629406.2, SFPQ, A_7, In, 11, driver, essential, high expression, KHERCERQIGK, (SEQ ID NO: 3868); XM_014122498.1, NUMA1, A_7, Del, 11, driver, essential, high expression, KTENIPLLRNA, (SEQ ID NO: 3869); XM_005631276.2, CKAP5, A_7, Del, 11, essential, high expression, NGKRGKKLWRL, (SEQ ID NO: 3870); XM_540739.5, FNBP4, A_7, In, 11, essential, high expression, SRVTSLGGRRW, (SEQ ID NO: 3871); XM_003639706.3, RIF1, A_7, In, 11, essential, high expression, NSFYCLEESNR, (SEQ ID NO: 3872); XM_863207.4, SNW1, A_7, In, 11, essential, high expression, NVKCIGHSGGC, (SEQ ID NO: 3873); XM_005638404.2, TRPM7, A_7, Del, 11, essential, high expression, KIRLQMDQNFF, (SEQ ID NO: 3874); XM_014115688.1, FERMT2, A_7, Del, 11, essential, high expression, KKNLLELHTTD, (SEQ ID NO: 3875); XM_005620224.2, USP1, A_7, In, 11, essential, high expression, RRSKKCGRIIY, (SEQ ID NO: 3876); XM_005623593.2, MED6, A_7, In, 11, essential, high expression, RGRTYTRNGKI, (SEQ ID NO: 3877); XM_014120337.1, BIRC6, A_7, Del, 11, essential, high expression, KRLIFLLLDTW, (SEQ ID NO: 3878); XM_014109172.1, PRKDC, A_7, In, 11, essential, high expression, KLVSPAKNTLR, (SEQ ID NO: 3879); XM_014109172.1, PRKDC, A_7, In, 11, essential, high expression, RRIMDSRNKRN, (SEQ ID NO: 3880); XM_014120145.1, PCM1, A_7, In, 11, driver, high expression, IYPSCYFNPSC, (SEQ ID NO: 3881); XM_014108499.1, KRAS, A_7, Del, 11, driver, high expression, KSTKKKRLLAV, (SEQ ID NO: 3882); XM_014113670.1, PTPRK, A_7, In, 11, driver, high expression, KIYMLEKTHLH, (SEQ ID NO: 3883); XM_005615442.2, AHI1, A_7, In, 11, high expression, NKEKKESSNFI, (SEQ ID NO: 3884); XM_014112822.1, ANK3, A_7, In, 11, high expression, MGYQRRKKCYN, (SEQ ID NO: 3885); XM_535783.4, ATP13A3, A_7, Del, 11, high expression, KLVFSVLVPKG, (SEQ ID NO: 3886); XM_845588.3, BRE, A_7, In, 11, high expression, KLSVQPQMGWK, (SEQ ID NO: 3887); XM_537686.5, COIL, A_7, Del, 11, high expression, KARGKTEPRVV, (SEQ ID NO: 3888); XM_005638410.2, DMXL2, A_7, Del, 11, high expression, KWMIYLQNQRF, (SEQ ID NO: 3889); XM_534306.5, EIF2A, A_7, In, 11, high expression, NAKLVSILVRR, (SEQ ID NO: 3890); XM_014106427.1, EPSTI1, A_7, In, 11, high expression, KISRKPQKRSI, (SEQ ID NO: 3891); XM_005622874.2, GALNT1, A_7, Del, 11, high expression, KRSEDFLLGMF, (SEQ ID NO: 3892); XM_014110420.1, GOLIM4, A_7, In, 11, high expression, KQRWRRTRSSR, (SEQ ID NO: 3893); XM_005617406.2, GPBP1, A_7, In, 11, high expression, KWMADAWKKWY, (SEQ ID NO: 3894); XM_014109639.1, LONP2, A_7, In, 11, high expression, NASVNARICSD, (SEQ ID NO: 3895); XM_014118119.1, PHIP, A_7, Del, 11, high expression, KVVEAENQKGR, (SEQ ID NO: 3896); XM_850281.4, POLE3, A_7, In, 11, high expression, NRCRGARQGQR, (SEQ ID NO: 3897); XM_014113435.1, RDX, A_7, Del, 11, high expression, KMSALRSNSRH, (SEQ ID NO: 3898); XM_014120231.1, ROCK2, A_7, In, 11, high expression, NQRSTDEGRRL, (SEQ ID NO: 3899); XM_005623486.2, SYNE2, A_7, Del, 11, high expression, KVSCKITFLSY, (SEQ ID NO: 3900); NM_001003154.2, TRDN, A_7, Del, 11, high expression, KLNIKKKNLQL, (SEQ ID NO: 3901); XM_005620337.1, USP24, A_7, In, 11, high expression, KQEGWIQVISA, (SEQ ID NO: 3902); XM_014118865.1, UTRN, A_7, Del, 11, high expression, KIGCLKVRKKQ, (SEQ ID NO: 3903); XM_005638476.2, VPS13C, A_7, In, 11, high expression, KFSSAESDCIL, (SEQ ID NO: 3904); XM_005640267.2, XIRP2, A_7, Del, 11, high expression, KMVFLHHCPQR, (SEQ ID NO: 3905); XM_005628995.2, ZCCHC11, A_7, Del, 11, high expression, KFQALLLLWTK, (SEQ ID NO: 3906); XM_014118132.1, ZNF484, A_7, Del, 11, high expression, NSRTMTHLEGV, (SEQ ID NO: 3907); XM_014114846.1, ZNF644, A_7, In, 11, high expression, THGILAFIIMC, (SEQ ID NO: 3908); XM_005628768.2, AASS, A_7, In, 11, high expression, KGLGSWIWLCI, (SEQ ID NO: 3909); XM_005618024.2, AP3B1, A_7, In, 11, high expression, NIPSAKTCTTA, (SEQ ID NO: 3910); XM_547771.4, CFL2, A_7, In, 11, high expression, KKESSSLLFKR, (SEQ ID NO: 3911); XM_535304.5, CHD9, A_7, In, 11, high expression, KAGKEEECRRC, (SEQ ID NO: 3912); XM_014106519.1, EPB41, A_7, Del, 11, high expression, KQSFLKHRRKQ, (SEQ ID NO: 3913); XM_014115573.1, EPB41L3, A_7, In, 11, high expression, TNSKWCLALFI, (SEQ ID NO: 3914); XM_543416.5, FBXO21, A_7, Del, 11, high expression, KFFTTYANRKF, (SEQ ID NO: 3915); XM_014117291.1, GOLM1, A_7, In, 11, high expression, KGPKPAAPSPQ, (SEQ ID NO: 3916); XM_014111650.1, GPRASP1, A_7, In, 11, high expression, RILDLAWRKGS, (SEQ ID NO: 3917); XM_843857.4, HNRNPD, A_7, In, 11, high expression, NFCWWPFSRYT, (SEQ ID NO: 3918); XM_005622209.2, KDM5B, A_7, Del, 11, high expression, KCGPPKRRKSN, (SEQ ID NO: 3919); XM_854556.3, NRD1, A_7, In 11, high expression, MLGSCAIWWKW, (SEQ ID NO: 3920); XM_005639981.2, RIPK1, A_7, Del, 11, high expression, KSIQTQPKIQL, (SEQ ID NO: 3921); XM_857524.4, TAOK3, A_7, Del, 11, high expression, KSFSKRHEMDP, (SEQ ID NO: 3922); XM_014107325.1, BRCA2, A_7, In, 11, driver, essential, NRSCSCGLFVR, (SEQ ID NO: 3923); XM_003434864.3, CREBBP, A_7, Del, 11, driver, essential, KSLSQRSYARL, (SEQ ID NO: 3924); XM_005629416.2, STIL, A_7, Del, 11, driver, essential, KACHAFYLVLS, (SEQ ID NO: 3925); XM_014116764.1, ATAD5, A_7, In, 11, essential, KETKEKDGNSR, (SEQ ID NO: 3926); XM_014116764.1, ATAD5, A_7, Del, 11, essential, KMRNRKICWIA, (SEQ ID NO: 3927); XM_005630416.1, BUB1, A_7, In, 11, essential, KCKFFWGLGSQ, (SEQ ID NO: 3928); XM_005616873.2, CD3EAP, A_7, In, 11, essential, KAEKATAGSCV, (SEQ ID NO: 3929); XM_005616873.2, CD3EAP, A_7, Del, 11, essential, KSGKSYSRILC, (SEQ ID NO: 3930); XM_535180.5, HSPA14, A_7, In, 11, essential, KWKVKIRSRYW, (SEQ ID NO: 3931); XM_543400.5, NAA25, A_7, In, 11, essential, KTGDHKETKNL, (SEQ ID NO: 3932); XM_005631123.2, NAT10, A_7, In, 11, essential, NKEWDVEHKAG, (SEQ ID NO: 3933); XM_536278.5, NCAPG, A_7, Del, 11, essential, KSKPFKVKMQK, (SEQ ID NO: 3934); XM_005641214.2, POLA1, A_7, Del, 11, essential, KVYLMYMLPSG, (SEQ ID NO: 3935); XM_005630719.2, POLR3C, A_7, Del, 11, essential, KPSVCLSNITW, (SEQ ID NO: 3936); XM_005626438.2, SRFBP1, A_7, In, 11, essential, NRCAKSCCQSL, (SEQ ID NO: 3937); XM_545078.5, ZBTB11, A_7, In, 11, essential, RKSKAEPGTGV, (SEQ ID NO: 3938); XM_014119418.1, CHD8, A_7, In, 11, essential, IYRGSGYKDHR, (SEQ ID NO: 3939); XM_014114838.1, RPAP2, A_7, In, 11, essential, KHNEKESWSES, (SEQ ID NO: 3940); XM_860879.4, VPS54, A_7, In, 11, essential, KVRGYRRKETS, (SEQ ID NO: 3941); XM_005619169.2, NSD1, A_7, In, 11, driver, KWRWHSEISTS, (SEQ ID NO: 3942); XM_536224.5, WHSC1, A_7, In, 11, driver, KNPHKEDTGAY, (SEQ ID NO: 3943); XM_534311.5, DHX36, A_7, In, 12, essential, high expression, NSSWCSENSNCY, (SEQ ID NO: 3944); NM_001003294.1, DLD, A_7, Del, 12, essential, high expression, KFQKRWLSLVQE, (SEQ ID NO: 3945); XM_005640003.2, PRPF4B, A_7, Del, 12, essential, high expression, NTVRMLVFILKL, (SEQ ID NO: 3946); XM_005636161.2, RSRC2, A_7, In, 12, essential, high expression, KENALARQKRRG, (SEQ ID NO: 3947); XM_014109056.1, SMC3, A_7, In, 12, essential, high expression, NSRTWITSPGSI, (SEQ ID NO: 3948); XM_843679.5, SON, A_7, Del, 12, essential, high expression, KKQIVFMESGFL, (SEQ ID NO: 3949); XM_014115688.1, FERMT2, A_7, In, 12, essential, high expression, RRTYWNCIQQTD, (SEQ ID NO: 3950); XM_542340.5, AKIRIN2, A_7, Del, 12, essential, high expression, KNSPYLLYDRLG, (SEQ ID NO: 3951); XM_536300.5, CCNH, A_7, Del, 12, essential, high expression, NMSHPDLKKLLL, (SEQ ID NO: 3952); XM_014115709.1, CCNK, A_7, Del, 12, essential, high expression, NVKISSKQLVVY, (SEQ ID NO: 3953); XM_005630347.2, CEBPZ, A_7, Del, 12, essential, high expression, KKMPAKKKQYLK, (SEQ ID NO: 3954); XM_005630347.2, CEBPZ, A_7, Del, 12, essential, high expression, NQSLLLGCTLII, (SEQ ID NO: 3955); XM_534762.5, MRPL40, A_7, Del, 12, essential, high expression, KGSDDWRKLARS, (SEQ ID NO: 3956); XM_005628224.2, PAICS, A_7, In, 12, essential, high expression, KYTKTTLWNTKK, (SEQ ID NO: 3957); XM_538283.5, PTPRB, A_7, In, 12, driver, high expression, SWPGSRSFSKEH, (SEQ ID NO: 3958); XM_014109909.1, ANK2, A_7, Del, 12, high expression, KMMRQSLQKHLS, (SEQ ID NO: 3959); XM_544889.6, BRWD1, A_7, Del, 12, high expression, KKISLKKRTCGG, (SEQ ID NO: 3960); XM_536179.5, CHD2, A_7, In, 12, high expression, KRPNNQNIGGSG, (SEQ ID NO: 3961); XM_545570.5, GLS, A_7, Del, 12, high expression, NLILEEKVVIKG, (SEQ ID NO: 3962); XM_014108478.1, KIF21A, A_7, Del, 12, high expression, KLGLNMKRNSKP, (SEQ ID NO: 3963); XM_535836.5, KNG1, A_7, Del, 12, high expression, KYLLTAQSWRCL, (SEQ ID NO: 3964); XM_005640290.2, PPIG, A_7, Del, 12, high expression, KRLREIKVHSQK, (SEQ ID NO: 3965); XM_014119398.1, RAPGEF2, A_7, Del, 12, high expression, KVKPILLEEGTS, (SEQ ID NO: 3966); XM_005623023.2, ROCK1, A_7, In, 12, high expression, KAEGRERSSREG, (SEQ ID NO: 3967); XM_014120231.1, ROCK2, A_7, Del, 12, high expression, KEIRTLKYPPRK, (SEQ ID NO: 3968); XM_014109413.1, SLTM, A_7, In, 12, high expression, NTSYCKKRRSRF, (SEQ ID NO: 3969); XM_005623031.2, SMCHD1, A_7, Del, 12, high expression, KWKIFKSWGIIH, (SEQ ID NO: 3970); XM_534172.5, TM9SF2, A_7, In, 12, high expression, KHVTELSTSLDC, (SEQ ID NO: 3971); XM_003639982.3, TNKS2, A_7, In, 12, high expression, TVYCSECQLQRH, (SEQ ID NO: 3972); XM_014109460.1, ZNF280D, A_7, Del, 12, high expression, KMDVIQIHLKAH, (SEQ ID NO: 3973); NM_001109813.1, ANGPTL1, A_7, In, 12, high expression, NNPKEIPSCHGC, (SEQ ID NO: 3974); XM_846354.4, BLOC1S2, A_7, Del, 12, high expression, NWKPSTRSWRSD, (SEQ ID NO: 3975); XM_846354.4, BLOC1S2, A_7, In, 12, high expression, TGSQVQEAGEAM, (SEQ ID NO: 3976); XM_543416.5, FBXO21, A_7, In, 12, high expression, NSLLPTPTENFK, (SEQ ID NO: 3977); XM_014119970.1, GIMAP4, A_7, Del, 12, high expression, KSKCYKNFTEQS, (SEQ ID NO: 3978); XM_005634030.1, IPO5, A_7, Del, 12, high expression, KFVILLQSWPGI, (SEQ ID NO: 3979); XM_533539.4, KIAA0020, A_7, In, 12, high expression, KQKHQQRNRNAT, (SEQ ID NO: 3980); XM_005639099.2, MRPL1, A_7, In, 12, high expression, KISKTNSKFYWP, (SEQ ID NO: 3981); XM_844495.4, OCIAD1, A_7, In, 12, high expression, RSQSKQIWRYLG, (SEQ ID NO: 3982); XM_014107327.1, PARP4, A_7, In, 12, high expression, KRLLLDRSTTRL, (SEQ ID NO: 3983); XM_014111645.1, RPS6KA3, A_7, Del, 12, high expression, KSQALMLDSFMP, (SEQ ID NO: 3984); XM_857524.4, TAOK3, A_7, In, 12, high expression, NPFPRDTKWTLK, (SEQ ID NO: 3985); XM_549626.5, TMCO1, A_7, In, 12, high expression, KENREARRETEK, (SEQ ID NO: 3986); XM_005634135.2, TMCO3, A_7, Del, 12, high expression, KTTSGCCFHCFV, (SEQ ID NO: 3987); XM_003433767.3, DIMT1, A_7, Del, 12, essential, KTTESTVRSIIL, (SEQ ID NO: 3988); XM_535180.5, HSPA14, A_7, In, 12, essential, KCSWGSSQSCWV, (SEQ ID NO: 3989); XM_005627636.2, MDN1, A_7, In, 12, essential, KSLAGLGIGPQS, (SEQ ID NO: 3990); XM_014108253.1, MRPS35, A_7, In, 12, essential, KYPTNTSPNESC, (SEQ ID NO: 3991); XM_014115086.1, NSL1, A_7, In, 12, essential, NCFNKPTVSDLF, (SEQ ID NO: 3992); XM_539419.4, RBM48, A_7, In, 12, essential, ITGEEGLYSKNY, (SEQ ID NO: 3993); XM_014118218.1, REV3L, A_7, Del, 12, essential, KIYLIFPTTCLR, (SEQ ID NO: 3994); XM_014117890.1, SMC2, A_7, In, 12, essential, NSCPENYRKKRG, (SEQ ID NO: 3995); XM_532882.5, SMC6, A_7, Del, 12, essential, NRRQFWTKKLID, (SEQ ID NO: 3996); XM_548321.5, TSR1, A_7, In, 12, essential, KPLSRPVQFGEW, (SEQ ID NO: 3997); XM_531673.4, YEATS4, A_7, Del, 12, essential, KQALKLQSLRRD, (SEQ ID NO: 3998); XM_534311.5, DHX36, A_7, Del, 13, essential, high expression, KEKWSRFTQKLMD, (SEQ ID NO: 3999); XM_014110821.1, DYNC1I2, A_7, Del, 13, essential, high expression, KLPQNMCFTASLL, (SEQ ID NO: 4000); XM_005626023.2, EIF5B, A_7, In, 13, essential, high expression, RTRSLCQNRTYPW, (SEQ ID NO: 4001); XM_014122447.1, PCF11, A_7, Del, 13, essential, high expression, NIFRIRLIAKMMM, (SEQ ID NO: 4002); XM_843679.5, SON, A_7, Del, 13, essential, high expression, KSIKDSQKNLSQR, (SEQ ID NO: 4003); XM_014122663.1, UPF2, A_7, Del, 13, essential, high expression, KKTRNGRKKKKKR, (SEQ ID NO: 4004); XM_003639330.3, CCT4, A_7, Del, 13, essential, high expression, KQDVMSSSYRSLS, (SEQ ID NO: 4005); XM_536509.5, TARS, A_7, Del, 13, essential, high expression, KSLRKNRLLKDWK, (SEQ ID NO: 4006); XM_014108264.1, PPFIBP1, A_7, In, 13, driver, high expression, KAEVNQIFNGQTF, (SEQ ID NO: 4007); XM_005627838.2, UBRS, A_7, In, 13, driver, high expression, KGRRRTECGARRS, (SEQ ID NO: 4008); XM_014120026.1, WHSC1L1, A_7, In, 13, driver, high expression, KLKKTSSLQTHQS, (SEQ ID NO: 4009); XM_005617569.2, CYLD, A_7, Del, 13, driver, high expression, KMRKLEFLQFSSY, (SEQ ID NO: 4010); XM_005617569.2, CYLD, A_7, In, 13, driver, high expression, NFSFFGVKYNRFT, (SEQ ID NO: 4011); XM_014108499.1, KRAS, A_7, In, 13, driver, high expression, NQQRRKDSWLCEN, (SEQ ID NO: 4012); XM_850902.4, G3BP2, A_7, In, 13, high expression, NKSCKRARNQRWW, (SEQ ID NO: 4013); XM_014112822.1, ANK3, A_7, In, 13, high expression, NPGEQAACVPGLC, (SEQ ID NO: 4014); XM_005616994.2, ARHGAP21, A_7, In, 13, high expression, KAHCYRNVRCQTG, (SEQ ID NO: 4015); NM_001168012.1, CCDC66, A_7, Del, 13, high expression, KPSGRKSLMNRLL, (SEQ ID NO: 4016); XM_531770.5, GCC2, A_7, In, 13, high expression, KDEVPSRRECSSV, (SEQ ID NO: 4017); XM_005640463.2, GLS, A_7, Del, 13, high expression, KSVFQKAQTWLVY, (SEQ ID NO: 4018); XM_005617406.2, GPBP1, A_7, In, 13, high expression, NLPSSSLRVSSES, (SEQ ID NO: 4019); XM_005617866.2, HP1BP3, A_7, Del, 13, high expression, KQYLPGLPFLPAS, (SEQ ID NO: 4020); XM_005637042.2, ITPR2, A_7, Del, 13, high expression, KSQKNSSKFSMTE, (SEQ ID NO: 4021); XM_005617055.2, KIAA1462, A_7, In, 13, high expression, NERDDILFGFHPS, (SEQ ID NO: 4022); XM_005636978.2, KIF21A, A_7, In, 13, high expression, ISEERIDWPSIYF, (SEQ ID NO: 4023); XM_014111164.1, MIA3, A_7, In, 13, high expression, TCSRNQETKYNSV, (SEQ ID NO: 4024); XM_548436.5, ODF2, A_7, In, 13, high expression, TPETISNQQPEIS, (SEQ ID NO: 4025); XM_014119574.1, OSBPL8, A_7, In, 13, high expression, KWSVGWNSSSECL, (SEQ ID NO: 4026); XM_005635475.2, PDS5B, A_7, In, 13, high expression, NTCHRFRREIRYG, (SEQ ID NO: 4027); XM_014106344.1, PHF11, A_7, In, 13, high expression, RNPQREEVDMCFL, (SEQ ID NO: 4028); XM_005623023.2, ROCK1, A_7, In, 13, high expression, KQRKFKENTRATK, (SEQ ID NO: 4029); XM_014112723.1, RYR2, A_7, In, 13, high expression, NEAAQELPADKWV, (SEQ ID NO: 4030); XM_843509.4, TTC17, A_7, Del, 13, high expression, NPKGIIRKLLGKR, (SEQ ID NO: 4031); XM_005638907.2, TTC3, A_7, In, 13, high expression, ISTRKNGRGPKGK, (SEQ ID NO: 4032); XM_855384.3, USP15, A_7, In, 13, high expression, KIVYIPVQQFRQY, (SEQ ID NO: 4033); XM_005628995.2, ZCCHC11, A_7, In, 13, high expression, NSKLFYFSGQSEY, (SEQ ID NO: 4034); XM_005630596.1, APOB, A_7, Del, 13, high expression, KIHWSLVMVCSSR, (SEQ ID NO: 4035); XM_014119381.1, ARHGAP10, A_7, Del, 13, high expression, KRSLTKRQKRTIV, (SEQ ID NO: 4036); XM_014107158.1, BCAS1, A_7, In, 13, high expression, RSGRHGIQSRKCL, (SEQ ID NO: 4037); XM_003434950.3, C4BPA, A_7, Del, 13, high expression, KSSVLGQRLEMEK, (SEQ ID NO: 4038); XM_005619370.2, C9, A_7, Del, 13, high expression, KLSKRRHQILKQI, (SEQ ID NO: 4039); XM_014115573.1, EPB41L3, A_7, Del, 13, high expression, NKFEVVLGTFHLM, (SEQ ID NO: 4040); XM_532929.5, FAM98A, A_7, Del, 13, high expression, KLKKEAVVRSFKS, (SEQ ID NO: 4041); XM_014107556.1, FDFT1, A_7, In, 13, high expression, KGSTATQLSLLPL, (SEQ ID NO: 4042); XM_536747.5, HIBADH, A_7, In, 13, high expression, SEERLTINRLQYY, (SEQ ID NO: 4043); XM_005628711.2, KBTBD2, A_7, In, 13, high expression, NKCRELCAIVEFC, (SEQ ID NO: 4044); XM_014118575.1, OXR1, A_7, Del, 13, high expression, KGEDINQLIINIL, (SEQ ID NO: 4045); XM_005619413.2, RAI14, A_7, Del, 13, high expression, NAKLHHLLSVLLS, (SEQ ID NO: 4046); XM_536302.5, RASA1, A_7, Del, 13, high expression, KAKENGGKIYILS, (SEQ ID NO: 4047); XM_014121157.1, SUV420H1, A_7, Del, 13, high expression, KTMQLLTENLQLV, (SEQ ID NO: 4048); XM_005629416.2, STIL, A_7, In, 13, driver, essential, KPAMLFTWFSHSR, (SEQ ID NO: 4049); XM_014116764.1, ATAD5, A_7, In, 13, essential, NEKKEAQRYNRSI, (SEQ ID NO: 4050); XM_014113312.1, CHEK1, A_7, Del, 13, essential, KLIQLLLLCCIKS, (SEQ ID NO: 4051); XM_014110473.1, ECT2, A_7, Del, 13, essential, KVLSKSSLIFVKN, (SEQ ID NO: 4052); XM_539084.5, REV3L, A_7, In, 13, essential, KFSEETSGNSQTE, (SEQ ID NO: 4053); XM_535878.4, RIOK1, A_7, In, 13, essential, KKGEDSQDEKGQI, (SEQ ID NO: 4054); XM_537096.5, RPF1, A_7, Del, 13, essential, KLFHSAFQEISQI, (SEQ ID NO: 4055); XM_005621512.2, SMG1, A_7, In, 13, essential, TFLNYTESPGNIA, (SEQ ID NO: 4056); XM_014112420.1, TICRR, A_7, In, 13, essential, KPYRSDPWQTGHF, (SEQ ID NO: 4057); XM_532047.5, CNTRL, A_7, Del, 13, driver, KFQKRRKTFFSSS, (SEQ ID NO: 4058); XM_005620109.2, SPECC1, A_7, Del, 13, driver, KGISCWHLKQPKV, (SEQ ID NO: 4059); XM_536371.4, TET1, A_7, Del, 13, driver, KLNPNHLCQSEAF, (SEQ ID NO: 4060); XM_536371.4, TET1, A_7, Del, 13, driver, KTLKIIYRVWLHD, (SEQ ID NO: 4061); XM_005619814.2, DDX10, A_7, In, 14, driver, essential, high expression, NIERFARSPVPFGN, (SEQ ID NO: 4062); XM_014121388.1, IWS1, A_7, In, 14, essential, high expression, KQICTRSENQHRGQ, (SEQ ID NO: 4063); XM_005631997.2, PRPF40A, A_7, In, 14, essential, high expression, KTAIRVSVCLRTFF, (SEQ ID NO: 4064); XM_846664.4, SEC62, A_7, In, 14, essential, high expression, RKRERKGRKWKRRR, (SEQ ID NO: 4065); XM_014106746.1, XRN1, A_7, In, 14, essential, high expression, SIRKRRNSSYRGQI, (SEQ ID NO: 4066); XM_542340.5, AKIRIN2, A_7, In, 14, essential, high expression, RTALIYSTTGWDDL, (SEQ ID NO: 4067); XM_858323.4, EIF2S2, A_7, Del, 14, essential, high expression, KQKRYLILMKLKKV, (SEQ ID NO: 4068); XM_542300.5, PRKRIR, A_7, Del, 14, essential, high expression, KLMKLLNRNPNIKK, (SEQ ID NO: 4069); XM_014113675.1, PTPRK, A_7, In, 14, driver, high expression, KEELLFLLLLPQAC, (SEQ ID NO: 4070); XM_014108062.1, DENR, A_7, In, 14, high expression, KEDCTTKSYDSQNS, (SEQ ID NO: 4071); XM_005639717.2, ABCC5, A_7, In, 14, high expression, RNQWFTEEITRQGS, (SEQ ID NO: 4072); XM_005615442.2, AHI1, A_7, In, 14, high expression, TEEKNRQIWRKNCT, (SEQ ID NO: 4073); XM_844507.4, AIDA, A_7, In, 14, high expression, KVHQHQVLCFHGDG, (SEQ ID NO: 4074); XM_014112822.1, ANK3, A_7, In, 14, high expression, KETPQTVPGSEEKV, (SEQ ID NO: 4075); XM_014112822.1, ANK3, A_7, Del, 14, high expression, NGLPETEKMLQLMP, (SEQ ID NO: 4076); XM_536179.5, CHD2, A_7, Del, 14, high expression, KEAQQSKYRGFRLT, (SEQ ID NO: 4077); XM_005635616.2, CLCN3, A_7, Del, 14, high expression, KGRCYQLPQLQGFL, (SEQ ID NO: 4078); XM_005617406.2, GPBP1, A_7, Del, 14, high expression, KMDGGCMEEMVLKT, (SEQ ID NO: 4079); XM_005640597.2, MAP2, A_7, Del, 14, high expression, KQKFRPTLPPGNSF, (SEQ ID NO: 4080); XM_535945.5, NOSTRIN, A_7, Del, 14, high expression, KTVSSVPGPGSQRA, (SEQ ID NO: 4081); XM_014118258.1, OGFRL1, A_7, Del, 14, high expression, KQKKQLKDSSWPIR, (SEQ ID NO: 4082); XM_005619375.2, OSMR, A_7, In, 14, high expression, KLHELLRRNPSWRK, (SEQ ID NO: 4083); XM_005639570.2, PARP14, A_7, In, 14, high expression, KRSISGFSPTVSGV, (SEQ ID NO: 4084); XM_532843.5, SNX25, A_7, Del, 14, high expression, KSSCLLLASCLSNL, (SEQ ID NO: 4085); XM_005623486.2, SYNE2, A_7, In, 14, high expression, KALFGYPANKKESG, (SEQ ID NO: 4086); XM_005638476.2, VPS13C, A_7, Del, 14, high expression, KLQRPQMRSSLIFY, (SEQ ID NO: 4087); XM_005640267.2, XIRP2, A_7, In, 14, high expression, KWFSSITVPRDDKG, (SEQ ID NO: 4088); XM_005635068.2, ZHX3, A_7, In, 14, high expression, NSYTQGEPPQSACG, (SEQ ID NO: 4089); XM_014110108.1, ABI3BP, A_7, In, 14, high expression, TSRNPRHAPPTLAT, (SEQ ID NO: 4090); XM_014115509.1, ACAA2, A_7, In, 14, high expression, NRTESQGHGFGRGE, (SEQ ID NO: 4091); XM_014106956.1, ADNP, A_7, Del, 14, high expression, KYLVTLGWNTVKNT, (SEQ ID NO: 4092); XM_003639068.2, ATMIN, A_7, In, 14, high expression, KENGELPAQPKVVQ, (SEQ ID NO: 4093); XM_539002.5, COL12A1, A_7, In, 14, high expression, NSIQRWQHNDGGCH, (SEQ ID NO: 4094); XM_537619.5, GPATCH8, A_7, Del, 14, high expression, KAVLPSLRAVSRWQ, (SEQ ID NO: 4095); XM_003639443.3, OXR1, A_7, Del, 14, high expression, KGEDINQLIINILW, (SEQ ID NO: 4096); XM_003431655.3, PNRC1, A_7, Del, 14, high expression, KSLLKTLRILKICI, (SEQ ID NO: 4097); XM_542038.5, RAD23A, A_7, In, 14, high expression, KRELGCQLPPESEL, (SEQ ID NO: 4098); XM_005619413.2, RAI14, A_7, In, 14, high expression, TQSSTTSCQSYSVE, (SEQ ID NO: 4099); XM_535303.4, RBL2, A_7, Del, 14, high expression, KSSLKEKKKILQGF, (SEQ ID NO: 4100); XM_005627829.2, RNF19A, A_7, In, 14, high expression, KKNFNRLSVSEEKG, (SEQ ID NO: 4101); XM_014107325.1, BRCA2, A_7, Del, 14, driver, essential, NGLEKENGMINQKE, (SEQ ID NO: 4102); XM_005624835.2, SUZ12, A_7, In, 14, driver, essential, KKGCKLSNKASSYR, (SEQ ID NO: 4103); XM_003640144.3, EIF2B5, A_7, In, 14, essential, KCICHDNDLQRVIP, (SEQ ID NO: 4104); XM_005615403.2, RNMT, A_7, In, 14, essential, KLRRRTQFSSGCPL, (SEQ ID NO: 4105); XM_844234.4, TAF1, A_7, Del, 14, essential, KPFLLTQKVASARG, (SEQ ID NO: 4106); XM_005628254.2, CENPC, A_7, In, 14, essential, KFFNIVFRNCKTKK, (SEQ ID NO: 4107); XM_003433299.3, NUP58, A_7, Del, 14, essential, KVIKQEQDQRIVKH, (SEQ ID NO: 4108); XM_005615936.2, CD274, A_7, In, 14, driver, TWENDGCGKMLHPR, (SEQ ID NO: 4109); XM_532056.6, ABCF1, A_7, In, 15, essential, high expression, KETRHPKRPKEEGCG, (SEQ ID NO: 4110); XM_547682.5, AFG3L2, A_7, In, 15, essential, high expression, NPGSAARGEEDRGIP, (SEQ ID NO: 4111); XM_014113043.1, TTC1, A_7, In, 15, essential, high expression, KHARRGETEKKRREH, (SEQ ID NO: 4112); XM_003640020.3, MRPL15, A_7, Del, 15, essential, high expression, KMVVLLLQLSMIQEV, (SEQ ID NO: 4113); XM_005627613.2, RARS2, A_7, In, 15, essential, high expression, KAFSASVPNASDHGI, (SEQ ID NO: 4114); XM_535608.5, SDAD1, A_7, Del, 15, essential, high expression, KRRRNQRCLTFQLFI, (SEQ ID NO: 4115); XM_014109172.1, PRKDC, A_7, Del, 15, essential, high expression, KIGIPGKKYITLRES, (SEQ ID NO: 4116); XM_014109172.1, PRKDC, A_7, In, 15, essential, high expression, KQHYSKVAPGFQSFS, (SEQ ID NO: 4117); XM_536845.5, BAZ1B, A_7, In, 15, high expression, RRKEMGSTKISASQI, (SEQ ID NO: 4118); NM_001168012.1, CCDC66, A_7, Del, 15, high expression, KMRLFHLSWMKINPL, (SEQ ID NO: 4119); XM_534852.5, ERGIC2, A_7, In, 15, high expression, NLKFGERVGCLSKGS, (SEQ ID NO: 4120); XM_005638175.2, FGF7, A_7, Del, 15, high expression, KRRKNKKQPTFFLWQ, (SEQ ID NO: 4121); XM_535836.5, KNG1, A_7, Del, 15, high expression, KFTLLSTVSHWERSY, (SEQ ID NO: 4122); XM_534296.5, PCOLCE2, A_7, Del, 15, high expression, NCLQLQYHLLPPRSL, (SEQ ID NO: 4123); XM_014112808.1, SYNE1, A_7, In, 15, high expression, NEDVGGFAKRHGKRP, (SEQ ID NO: 4124); XM_014112808.1, SYNE1, A_7, Del, 15, high expression, NSFMKTRVNAVDLLT, (SEQ ID NO: 4125); XM_531794.5, TMEM131, A_7, Del, 15, high expression, KIYYPKSLLSSPGLL, (SEQ ID NO: 4126); XM_005633672.2, USP47, A_7, In, 15, high expression, NMEESWHCLFGLSYL, (SEQ ID NO: 4127); XM_014110169.1, ACAP2, A_7, Del, 15, high expression, KIFLKWLRLWLMARM, (SEQ ID NO: 4129); XM_005630596.1, APOB, A_7, In, 15, high expression, KVLQTHFGHSEQENH, (SEQ ID NO: 4130); XM_005629294.2, CCDC53, A_7, Del, 15, high expression, KIQKKVQTVNLLLVT, (SEQ ID NO: 4131); XM_843857.4, HNRNPD, A_7, Del, 15, high expression, KFLLVAFLQIHLKKK, (SEQ ID NO: 4132); XM_014108547.1, LRRK2, A_7, In, 15, high expression, KHYVGRMWYKTFLIF, (SEQ ID NO: 4133); XM_846278.4, MAP4K5, A_7, Del, 15, high expression, KMVATTSSVISGQWE, (SEQ ID NO: 4134); XM_542763.5, NR1D2, A_7, Del, 15, high expression, NIVSMIYTQWEQGIC, (SEQ ID NO: 4135); XM_537467.4, PPM1A, A_7, Del, 15, high expression, KGQRGLRKNRRKGEK, (SEQ ID NO: 4136); XM_531828.5, RTN4, A_7, In, 15, high expression, KDRRKTGPNCNREEF, (SEQ ID NO: 4137); XM_005640326.2, ZAK, A_7, In, 15, high expression, KREINHSKQLPQKFC, (SEQ ID NO: 4138); XM_014107325.1, BRCA2, A_7, In, 15, driver, essential, NRNYKGIFGQSKKSF, (SEQ ID NO: 4139); XM_536875.4, TRRAP, A_7, In, 15, driver, essential, NTGRYLLSPLCRRAA, (SEQ ID NO: 4140); XM_847666.4, PPM1D, A_7, In, 15, driver, essential, KFKNVNSRSNESPRS, (SEQ ID NO: 4141); XM_539598.4, CLSPN, A_7, In, 15, essential, KDGKNQAAKKEGNEK, (SEQ ID NO: 4142); XM_535898.5, NUP153, A_7, Del, 15, essential, KKKCLPPKEGSLSVV, (SEQ ID NO: 4143); NM_001003057.1, TCOF1, A_7, In, 15, essential, KGKEEEGKKGLNQRP, (SEQ ID NO: 4144); XM_532318.5, TAF2, A_7, Del, 15, essential, KLLVLMNTAIGLKRS, (SEQ ID NO: 4145); XM_005625969.2, SEPT5, A_7, In, 15, driver, KGKYSFFNYVWPCWL, (SEQ ID NO: 4146); XM_532047.5, CNTRL, A_7, In, 15, driver, NFRSGERPSFQAVEW, (SEQ ID NO: 4147); XM_005619382.2, NIPBL, A_7, In, 16, essential, high expression, KKTRFLPTGGWGCYRR, (SEQ ID NO: 4148); XM_533359.5, PRPF40A, A_7, Del, 16, essential, high expression, KKRSKQSSYEREIGKP, (SEQ ID NO: 4149); XM_005638404.2, TRPM7, A_7, Del, 16, essential, high expression, KHLTLARVKQFIYFKH, (SEQ ID NO: 4150); XM_014114127.1, CCT6A, A_7, In, 16, essential, high expression, KSRRCIHPHMQCVLRV, (SEQ ID NO: 4151); XM_533327.6, DDX18, A_7, Del, 16, essential, high expression, KQWKLYKLERKKRNLP, (SEQ ID NO: 4152); XM_014120532.1, ZC3H8, A_7, Del, 16, essential, high expression, KTKTLKLFRKMVNRRK, (SEQ ID NO: 4154); XM_014120026.1, WHSC1L1, A_7, Del, 16, driver, high expression, KTQENLLLTNTSKLTK, (SEQ ID NO: 4155); XM_014107827.1, CLIP1, A_7, In, 16, driver, high expression, KGGNEPQPVPGSESQV, (SEQ ID NO: 4156); XM_544163.4, CPNE3, A_7, Del, 16, high expression, KRDKRRKATRIQVLSV, (SEQ ID NO: 4157); XM_014106605.1, GOLGA4, A_7, Del, 16, high expression, KRWKKLSRKQKRCKKR, (SEQ ID NO: 4158); XM_537751.5, NUFIP2, A_7, Del, 16, high expression, KGKLGAIVPRVVKTLI, (SEQ ID NO: 4159); XM_005639570.2, PARP14, A_7, Del, 16, high expression, KEKHLRLLPNSLWSLN, (SEQ ID NO: 4161); XM_005615469.2, PHACTR2, A_7, In, 16, high expression, NTCASKRGCCWGKPQG, (SEQ ID NO: 4162); XM_014111292.1, RGS2, A_7, In, 16, high expression, NQVTPKAVLQSKENIY, (SEQ ID NO: 4163); XM_853581.4, SEC31A, A_7, Del, 16, high expression, KSMLLRLNLKNMCGPF, (SEQ ID NO: 4164); XM_005618098.2, TTC37, A_7, In, 16, high expression, KPCTKDHSESSSSFSR, (SEQ ID NO: 4166); XM_859377.4, UPF3B, A_7, Del, 16, high expression, KSRKVVLAIEKKEERS, (SEQ ID NO: 4167); XM_005638476.2, VPS13C, A_7, Del, 16, high expression, NTGSCSRVIKVPIRSS, (SEQ ID NO: 4168); XM_532445.5, WASL, A_7, In, 16, high expression, IPKSSYRLVGPATKEI, (SEQ ID NO: 4169); XM_014112483.1, ZFAND6, A_7, In, 16, high expression, KESLFHVQEESGTYWV, (SEQ ID NO: 4170); XM_005630575.2, ZNF638, A_7, In, 16, high expression, SIRRCSTTIWACFRIQ, (SEQ ID NO: 4171); XM_536302.5, RASA1, A_7, In, 16, high expression, RQRKTVEKFIFYLRRQ, (SEQ ID NO: 4173); XM_014108798.1, SORBS1, A_7, Del, 16, high expression, KRQRMTAGGWLRALRT, (SEQ ID NO: 4174); XM_014117553.1, USP34, A_7, Del, 16, high expression, KIWLILMVKNLGVKRN, (SEQ ID NO: 4175); XM_537657.5, CWC25, A_7, In, 16, essential, KGEEEKEGKEKEAQET, (SEQ ID NO: 4176); XM_005625951.2, POLR3B, A_7, In, 16, essential, KQNQYGCETGAILFET, (SEQ ID NO: 4177); XM_849077.4, PPAT, A_7, Del, 16, essential, NLEYCRTTLKAKELFL, (SEQ ID NO: 4178); XM_537096.5, RPF1, A_7, In, 16, essential, NYSTVHFKRFHRSDSY, (SEQ ID NO: 4180); XM_005617106.2, RPP38, A_7, Del, 16, essential, NKAQTNVAQKLIRARI, (SEQ ID NO: 4181); XM_005633939.2, SUGT1, A_7, In, 16, essential, KLCCCFRNFYRRTEIR, (SEQ ID NO: 4182); XM_534297.5, U2SURP, A_7, Del, 16, essential, KILQISHPMKDHRLFL, (SEQ ID NO: 4183); XM_005629808.2, KAT6A, A_7, In, 16, driver, TNSSPKKEGPKTQTPQ, (SEQ ID NO: 4184); XM_014113014.1, RANBP17, A_7, In, 16, driver, TSEDRCCEIYAKKPHE, (SEQ ID NO: 4185); XM_535474.5, USP8, A_7, Del, 17, driver, essential, high expression, KMRPQRKGQSQQRSCIQ, (SEQ ID NO: 4186); XM_852177.4, SMARCB1, A_7, Del, 17, driver, essential, high expression, KQNLTLRIMDTQPWPPA, (SEQ ID NO: 4187); XM_844375.4, EPRS, A_7, Del, 17, essential, high expression, KKKILQIGILRSSQSQK, (SEQ ID NO: 4188); XM_014121388.1, IWS1, A_7, In, 17, essential, high expression, KASTEKINIITYCGYAP, (SEQ ID NO: 4189); XM_005624952.2, PAFAH1B1, A_7, In, 17, essential, high expression, KWQAWAILTVWIQRQDY, (SEQ ID NO: 4190); XM_005621408.2, RBBP6, A_7, Del, 17, essential, high expression, KLEVQKMYLIQKNPLKN, (SEQ ID NO: 4192); XM_546107.5, TFAM, A_7, Del, 17, essential, high expression, KSMKMLIGQTGRHTKKR, (SEQ ID NO: 4193); XM_014113043.1, TTC1, A_7, Del, 17, essential, high expression, KTCQKRRNRKEEKRALD, (SEQ ID NO: 4194); XM_846653.4, DDX21, A_7, Del, 17, essential, high expression, KQRSPLKRKLFLLKQKK, (SEQ ID NO: 4195); XM_014115771.1, NGDN, A_7, Del, 17, essential, high expression, KFKLEPILQRRVSASWK, (SEQ ID NO: 4196); XM_536845.5, BAZ1B, A_7, Del, 17, high expression, KEKGNGLHQNFCLTNMM, (SEQ ID NO: 4197); XM_005635615.2, CLCN3, A_7, Del, 17, high expression, KISSVIWPRRQTKTPLQ, (SEQ ID NO: 4198); XM_005638175.2, FGF7, A_7, In, 17, high expression, NEERTKNSPLSSYGNNI, (SEQ ID NO: 4199); XM_014110232.1, GOLGB1, A_7, Del, 17, high expression, KEMWKPSNKLSWRRISK, (SEQ ID NO: 4200); XM_544755.5, MYO9A, A_7, Del, 17, high expression, KTLMDQSIMVVCSHGYG, (SEQ ID NO: 4201); XM_548436.5, ODF2, A_7, Del, 17, high expression, NSRNHQQPTARNLISAE, (SEQ ID NO: 4202); XM_005619375.2, OSMR, A_7, Del, 17, high expression, KQDTPRSWLLRTTLEWS, (SEQ ID NO: 4203); XM_014108413.1, PLEKHA5, A_7, Del, 17, high expression, KLRTFFPKRSSNLNQME, (SEQ ID NO: 4204); XM_859168.4, SLTM, A_7, Del, 17, high expression, KPARLLKRKEAIQIILN, (SEQ ID NO: 4205); XM_860876.4, SPAG9, A_7, In, 17, high expression, KGFTDPSGIFRISNKTT, (SEQ ID NO: 4206); XM_003433632.3, TACC2, A_7, In, 17, high expression, NTRETRQHSCLAYQIPR, (SEQ ID NO: 4207); XM_014111638.1, ZFX, A_7, Del, 17, high expression, KCTSVDIVTLRLQIHLF, (SEQ ID NO: 4209); XM_014106956.1, ADNP, A_7, Del, 17, high expression, KSSPTNASIALVYTPAT, (SEQ ID NO: 4210); XM_014112204.1, CAST, A_7, In, 17, high expression, RRYHSASSRLFETHGAQ, (SEQ ID NO: 4211); XM_005626522.2, KIF3A, A_7, Del, 17, high expression, KEGAVAAAVVQTPHVLS, (SEQ ID NO: 4212); XM_014110285.1, PARP9, A_7, In, 17, high expression, NYLDTSRFSVGTGHKRI, (SEQ ID NO: 4213); XM_535180.5, HSPA14, A_7, Del, 17, essential, KMLLGKQPELLGLMFCG, (SEQ ID NO: 4214); XM_014122593.1, MCM10, A_7, Del, 17, essential, KGENNLPTWNLKNFRKF, (SEQ ID NO: 4215); XM_014115086.1, NSL1, A_7, Del, 17, essential, KLFQQTHSVRFVLATMS, (SEQ ID NO: 4216); XM_539084.5, REV3L, A_7, Del, 17, essential, KIFRRDFRKFSNRMISL, (SEQ ID NO: 4217); XM_005625106.2, SEC16A, A_7, In, 17, essential, KDRSLFARRQEQIDRLG, (SEQ ID NO: 4218); XM_534297.5, U2SURP, A_7, In, 17, essential, KQFGTLQRRIETNSGRA, (SEQ ID NO: 4219); XM_005624373.2, NMT1, A_7, Del, 17, essential, KRRKRKAMRQIQPRISL, (SEQ ID NO: 4220); XM_014120446.1, EML4, A_7, In, 17, driver, KERKTTRTEGKKRGISF, (SEQ ID NO: 4221); XM_005622378.2, CEP350, A_7, In, 18, essential, high expression, RKDRMAGFTHWKCSELTS, (SEQ ID NO: 4222); NM_001287118.1, CFDP1, A_7, Del, 18, essential, high expression, KERLRAFRPGRENKVASH, (SEQ ID NO: 4223); XM_844375.4, EPRS, A_7, In, 18, essential, high expression, RRKSCRLVFSGHHKVRND, (SEQ ID NO: 4224); XM_860109.5, RBM39, A_7, Del, 18, essential, high expression, KTELQQWQTIYKREVLDL, (SEQ ID NO: 4225); XM_014109056.1, SMC3, A_7, Del, 18, essential, high expression, KFENLDHFPRKHLKSTRH, (SEQ ID NO: 4226); XM_843366.4, CHD1, A_7, In, 18, essential, high expression, RSSETFWCRRVKEEKSKS, (SEQ ID NO: 4227); XM_014112852.1, CCAR1, A_7, Del, 18, high expression, KIWPFLIHQMLTIYTVQR, (SEQ ID NO: 4229); XM_005624991.1, MED13, A_7, In, 18, high expression, KNSKALGSSRSSHLATVS, (SEQ ID NO: 4230); XM_537461.5, ARID4A, A_7, In, 18, high expression, KAETENTGTVITREKNKN, (SEQ ID NO: 4231); XM_014115338.1, ASH1L, A_7, In, 18, high expression, NYKEKQWTINENNYPQNK, (SEQ ID NO: 4232); XM_536312.4, CMYA5, A_7, Del, 18, high expression, KEILLNLKMFQQTQKIIL, (SEQ ID NO: 4233); XM_014115252.1, EFCAB2, A_7, Del, 18, high expression, KSKTLLKCLTTRRIIQWM, (SEQ ID NO: 4234); XM_005622874.2, GALNT1, A_7, In, 18, high expression, KGARTSCWGCSRASTKAS, (SEQ ID NO: 4235); XM_014106605.1, GOLGA4, A_7, Del, 18, high expression, KISTIKKLQSWLKNTKQN, (SEQ ID NO: 4236); XM_857278.5, HELZ, A_7, Del, 18, high expression, KIAQLFITMQRYSKWWNE, (SEQ ID NO: 4237); XM_537881.5, OSBPL1A, A_7, Del, 18, high expression, KTKFCQKHWRLWPPSIMN, (SEQ ID NO: 4238); XM_005619375.2, OSMR, A_7, In, 18, high expression, NRILPGAGSFGQPSSGHE, (SEQ ID NO: 4239); XM_005627518.2, PHF3, A_7, Del, 18, high expression, NKLRKIQKFRVAILVLNL, (SEQ ID NO: 4240); XM_535547.4, SCAPER, A_7, In, 18, high expression, KECSRSDCISSCWWINSP, (SEQ ID NO: 4241); XM_535304.5, CHD9, A_7, Del, 18, high expression, NTQKMQKGNNLKKRFKVL, (SEQ ID NO: 4242); XM_005622209.2, KDM5B, A_7, In, 18, high expression, NHQCCGSVCLPLVWQWQR, (SEQ ID NO: 4243); XM_005615397.2, ME2, A_7, In, 18, high expression, NEWPFGKVHLYNGNTRKK, (SEQ ID NO: 4244); NM_001286960.1, PLG, A_7, Del, 18, high expression, KTTAVILMVMSMVLGATQ, (SEQ ID NO: 4245); XM_005622961.2, PRG4, A_7, In, 18, high expression, TSHVPQAPCVNSYIYPRD, (SEQ ID NO: 4246); XM_005638171.2, UACA, A_7, Del, 18, high expression, KLERQKESMKNHLLKSDS, (SEQ ID NO: 4247); XM_005625193.2, GTF3C4, A_7, Del, 18, essential, KRLKAVEPPIFGVSNFSS, (SEQ ID NO: 4248); XM_014106867.1, MYBL2, A_7, Del, 18, essential, KGVLPCPLSQRTAPACPS, (SEQ ID NO: 4249); XM_005620578.2, ZC3H18, A_7, Del, 18, essential, KQPFEKNRSLILKKKGLR, (SEQ ID NO: 4250); XM_535063.5, MCM4, A_7, Del, 18, essential, KPPLKISSCLTRYYQGLI, (SEQ ID NO: 4251); XM_005624435.2, PSME3, A_7, In, 18, essential, NKVFTVWGSLSFSLQLQG, (SEQ ID NO: 4252); XM_847556.4, BRIP1, A_7, In, 18, driver, KCDFQIHKPNLQQTNKAS, (SEQ ID NO: 4253); XM_536371.4, TET1, A_7, Del, 18, driver, KENVLLSPYLVLERRGQQ, (SEQ ID NO: 4254); XM_544656.5, EIF3J, A_7, In, 19, essential, high expression, NSRENKRERTATEEKARRN, (SEQ ID NO: 4255); XM_014106743.1, XRN1, A_7, Del, 19, essential, high expression, KHKGYAHQKKPHFTSYTCL, (SEQ ID NO: 4256); XM_536972.4, RSL1D1, A_7, Del, 19, essential, high expression, KLPTPPNSGPKSAKYPSQP, (SEQ ID NO: 4257); XM_536972.4, RSL1D1, A_7, In, 19, essential, high expression, SSPHPQTVAQKAQSTPVNL, (SEQ ID NO: 4258); XM_535952.5, SSB, A_7, In, 19, essential, high expression, NHRRSTRISKQMEIKRSQI, (SEQ ID NO: 4259); XM_014110816.1, USP48, A_7, Del, 19, essential, high expression, KIQMCGTLSNSSSVENMLT, (SEQ ID NO: 4260); XM_014118978.1, AKAP9, A_7, Del, 19, driver, high expression, KILKLTIRSYRRTMLAFSR, (SEQ ID NO: 4261); XM_014111218.1, ZC3H11A, A_7, In, 19, high expression, NRGFATYSYQQRTIRGAHR, (SEQ ID NO: 4262); XM_005639717.2, ABCC5, A_7, Del, 19, high expression, KKPVVHRRNHKTRVLKQDQ, (SEQ ID NO: 4263); XM_005617034.2, ARHGAP12, A_7, In, 19, high expression, NQEKLKEVSYTAPHFASCS, (SEQ ID NO: 4264); XM_014113549.1, CHD3, A_7, Del, 19, high expression, KRRRRQSGGKKGREMGDKR, (SEQ ID NO: 4265); XM_544163.4, CPNE3, A_7, In, 19, high expression, KETKEEKLQEFRCYQCETV, (SEQ ID NO: 4266); XM_005618961.2, DDX50, A_7, Del, 19, high expression, KAAHQRFLFWLLQGNWQTK, (SEQ ID NO: 4267); XM_005623378.2, KLHDC2, A_7, Del, 19, high expression, KLTQKEMFLLPCQEAVLCV, (SEQ ID NO: 4268); XM_014119574.1, OSBPL8, A_7, Del, 19, high expression, KLIIQRFSGTQHQTLSSGD, (SEQ ID NO: 4270); XM_005615470.1, PHACTR2, A_7, In, 19, high expression, NRQAANLSIVLRCNHLWHV, (SEQ ID NO: 4271); XM_005627518.2, PHF3, A_7, Del, 19, high expression, KLKLLLEYLVNIQIMKQKV, (SEQ ID NO: 4272); XM_005627518.2, PHF3, A_7, In, 19, high expression, NITRSKFSAGFQTLNSFFE, (SEQ ID NO: 4273); XM_853101.4, RBM26, A_7, In, 19, high expression, RGDNKGGRAREEVFQKAKS, (SEQ ID NO: 4274); XM_014111292.1, RGS2, A_7, Del, 19, high expression, KPSHPKSCPPKQGKYILIS, (SEQ ID NO: 4275); XM_005623031.2, SMCHD1, A_7, In, 19, high expression, NGKYSKVGELYIEITSCIE, (SEQ ID NO: 4276); NM_001038648.1, TMEM57, A_7, In, 19, high expression, KEEVGRSYCCPGRCICCCI, (SEQ ID NO: 4277); XM_534241.4, TOP2B, A_7, In, 19, high expression, KSRFWESLLISFILSEVRR, (SEQ ID NO: 4278); XM_005630608.2, CTSS, A_7, In, 19, high expression, KSKICDASQSGTLNGNAFI, (SEQ ID NO: 4279); XM_532364.5, IREB2, A_7, Del, 19, high expression, NTVPGTPETGLPKDRICWA, (SEQ ID NO: 4280); XM_535195.5, ITIH2, A_7, In, 19, high expression, KCEAAHTRQHLLVQLGDRI, (SEQ ID NO: 4281); XM_014120511.1, KRCC1, A_7, Del, 19, high expression, NQRRNSPSIREKKVVRNQI, (SEQ ID NO: 4282); XM_535817.5, MCCC1, A_7, Del, 19, high expression, KWVYGPQLCIVRPTGIPCM, (SEQ ID NO: 4283); XM_014121449.1, RALB, A_7, In, 19, high expression, NVREQRQEWQEKQQEQEKF, (SEQ ID NO: 4284); XM_005639981.2, RIPK1, A_7, In, 19, high expression, RVSRPSPKFSCEENAVSPD, (SEQ ID NO: 4285); XM_005623792.2, SETD3, A_7, Del, 19, high expression, NKKVCLLLLMEKEKITFLT, (SEQ ID NO: 4286); XM_014108806.1, SORBS1, A_7, In, 19, high expression, RSRPLPSRVRGTFRKHGDT, (SEQ ID NO: 4287); XM_014116764.1, ATAD5, A_7, Del, 19, essential, NEVSTKYFLNVIVNKKVHN, (SEQ ID NO: 4288); XM_005638394.2, CEP152, A_7, In, 19, essential, KWNTWKRIGREDSFSSERA, (SEQ ID NO: 4289); XM_014121289.1, ELP4, A_7, In, 19, essential, TEKHFKNRNPESWITFMGR, (SEQ ID NO: 4290); XM_014115954.1, SNAPC1, A_7, In, 19, essential, KKQRNIEIRRKEDAFQKQR, (SEQ ID NO: 4291); XM_547651.5, THOC1, A_7, In, 19, essential, NGRIKNRRRTCIFCKIFNK, (SEQ ID NO: 4292); XM_005624533.2, TOP2A, A_7, Del, 19, essential, KHKWLKFCLLLAEKESSPE, (SEQ ID NO: 4293); XM_537623.5, LSM12, A_7, In, 19, essential, KHRSHGRSCYYTPISSGKL, (SEQ ID NO: 4294); XM_538677.5, MLLT3, A_7, Del, 19, driver, KGKRVAQRLYLKVFLAHHH, (SEQ ID NO: 4295); XM_005621408.2, RBBP6, A_7, Del, 20, essential, high expression, KLTTTTESTQVPNVEMKGMN, (SEQ ID NO: 4297); XM_005620768.2, NFAT5, A_7, Del, 20, essential, high expression, KALVPDWFLELTSQGKMAPL, (SEQ ID NO: 4298); NM_001286231.1, TAF9, A_7, In, 20, essential, high expression, KGINFCGKNNSSTVKCWFCY, (SEQ ID NO: 4299); XM_014120154.1, FAT1, Del, 20, driver, high expression, KILMLKHERRSGCKCWIQMT, (SEQ ID NO: 4300); XM_014120026.1, WHSC1L1, A_7, Del, 20, driver, high expression, KATSMTHVDLKSASHTKSPN, (SEQ ID NO: 4301); XM_014115338.1, ASH1L, A_7, In, 20, high expression, NPFKRLRGCQTPFWLRGHHL, (SEQ ID NO: 4302); XM_014113564.1, CHD3, A_7, Del, 20, high expression, KRRRRQSGGKKGREMGDKSR, (SEQ ID NO: 4304); XM_014118080.1, DST, A_7, In, 20, high expression, KEMSFLLYHCVLCFQGHSWK, (SEQ ID NO: 4305); XM_003639377.3, FAM13B, A_7, In, 20, high expression, NDKRSFDRRENFSPKKSSLL, (SEQ ID NO: 4306); XM_538557.5, FEM1C, A_7, In, 20, high expression, KRSTWGLKILEKGNEHEVQR, (SEQ ID NO: 4307); XM_005626399.1, HSPA4, A_7, In, 20, high expression, KRRAEWTSGRTRRQPRPPGC, (SEQ ID NO: 4308); XM_005629477.2, LUC7L2, A_7, In, 20, high expression, NTGSIGENGRYYSSTNQIQR, (SEQ ID NO: 4310); XM_535242.6, PLK2, A_7, In, 20, high expression, NSSLLCRAWPVLCFSPNRCS, (SEQ ID NO: 4311); XM_014120231.1, ROCK2, A_7, In, 20, high expression, KKSELSNIHRESESAPETTG, (SEQ ID NO: 4312); XM_005635161.2, STAU1, A_7, Del, 20, high expression, NQCISLSTLTLGCSPPITTT, (SEQ ID NO: 4313); XM_005635807.1, TRIP12, A_7, In, 20, high expression, TYCEQRGAAETSGVCNAGSR, (SEQ ID NO: 4314); XM_014114846.1, ZNF644, A_7, In, 20, high expression, KDFHERLCCRIIQKISHLYM, (SEQ ID NO: 4315); XM_014112204.1, CAST, A_7, Del, 20, high expression, KKVSQCLLQTLRNPWGPMMP, (SEQ ID NO: 4317); XM_005634496.2, CEP70, A_7, Del, 20, high expression, KRQRTQRNQMNPTEITSSRP, (SEQ ID NO: 4318); XM_014117434.1, IL1R1, A_7, Del, 20, high expression, KMSFPKYSGIRIANLCFLTI, (SEQ ID NO: 4320); XM_540963.4, KIAA1109, A_7, Del, 20, high expression, NWVLHYRMKRKRKEKTKKNT, (SEQ ID NO: 4321); XM_531828.5, RTN4, A_7, Del, 20, high expression, KPLLIQNKRTDHHLLYFQQS, (SEQ ID NO: 4322); XM_536334.4, HEATR1, A_7, Del, 20, essential, NKSFFISLFLFLQVEGSISF, (SEQ ID NO: 4323); XM_846123.4, POLE2, A_7, In, 20, essential, ISSSINDQPPCTKFIWNSKG, (SEQ ID NO: 4324); XM_014118218.1, REV3L, A_7, Del, 20, essential, KIVEQERRLRNRKLNLLIPL, (SEQ ID NO: 4325); XM_005621851.2, RNPC3, A_7, Del, 20, essential, KRKDLMTLWKMIKKRKNLVV, (SEQ ID NO: 4326); XM_003638871.2, PTCH1, A_7, Del, 20, driver, KTAASSWLWASSYLGPSLWD, (SEQ ID NO: 4327); XM_005632455.2, GNL3, A_7, In, 21, essential, high expression, KGSRAPSKIEEGGQKARSQKA, (SEQ ID NO: 4328); XM_014112182.1, AHI1, A_7, Del, 21, high expression, KMKQNSSQRHLLLPPPSASMG, (SEQ ID NO: 4329); XM_014112822.1, ANK3, A_7, Del, 21, high expression, KGNTANGPGIGRKSLMPMQVT, (SEQ ID NO: 4330); XM_014112822.1, ANK3, A_7, Del, 21, high expression, KSRRASCLCTRSLLEKSSRRP, (SEQ ID NO: 4331); XM_005634418.2, ATP2C1, A_7, Del, 21, high expression, KQANYQSVKLQAFSKLIFRMV, (SEQ ID NO: 4332); XM_005635616.2, CLCN3, A_7, Del, 21, high expression, KDILRHMAQTANQDPASIMFN, (SEQ ID NO: 4333); XM_005635615.2, CLCN3, A_7, In, 21, high expression, RYPPSYGPDGKPRPRFNNVQL, (SEQ ID NO: 4334); XM_532831.5, FGL1, A_7, Del, 21, high expression, KTAAMHNINFSKLEMKRIPMS, (SEQ ID NO: 4335); XM_005631810.2, LARP1B, A_7, Del, 21, high expression, KCMRNLDNLLGKMQKKITGMD, (SEQ ID NO: 4336); XM_005640597.2, MAP2, A_7, In, 21, high expression, NRSSGPLSLQEIHFKTCYQIY, (SEQ ID NO: 4337); XM_005635475.2, PDS5B, A_7, Del, 21, high expression, KVKEADHLNLLVEVHQRKSQQ, (SEQ ID NO: 4338); XM_005615470.1, PHACTR2, A_7, Del, 21, high expression, KPASSQPLDRPQMQPPLARLT, (SEQ ID NO: 4339); XM_005623023.2, ROCK1, A_7, Del, 21, high expression, KFCSIMMNKIRNNPIHPWYWT, (SEQ ID NO: 4340); XM_014119514.1, ZMYM4, A_7, Del, 21, high expression, KTFLNSLIRFLSLNQYGKILV, (SEQ ID NO: 4341); XM_863511.5, CDC42BPB, A_7, In, 21, high expression, NGRKIQSRYWTQTSRFSGFNI, (SEQ ID NO: 4342); XM_005633338.2, CWC15, A_7, Del, 21, high expression, KTETVQQENIQPPRRCQRSLG, (SEQ ID NO: 4343); XM_014118822.1, REST, A_7, Del, 21, high expression, KAASLLRKRLLRRSLLRRGLL, (SEQ ID NO: 4344); XM_014110770.1, UBR3, A_7, Del, 21, high expression, NCLSLKRSRFTLGIHVQQFMM, (SEQ ID NO: 4345); XM_014107325.1, BRCA2, A_7, Del, 21, driver, essential, KILMTVESISLRKITPIKQQL, (SEQ ID NO: 4346); XM_005638394.2, CEP152, A_7, Del, 21, essential, KVEYLEKNWKRRFILFRESLS, (SEQ ID NO: 4347); XM_542610.5, DIS3, A_7, In, 21, essential, KKNRKRGFDSFFSRSSIPHGQ, (SEQ ID NO: 4348); XM_014110474.1, ECT2, A_7, In, 21, essential, RRTSQIGDIGSSHGWSYSKRL, (SEQ ID NO: 4349); XM_536459.3, GEMIN5, A_7, Del, 21, essential, KGSLNLSQSPKRRKSPPQELL, (SEQ ID NO: 4350); XM_005627636.2, MDN1, A_7, In, 21, essential, RAVSCLKKPVYRNMVPTKHKS, (SEQ ID NO: 4351); XM_014115880.1, MIS18BP1, A_7, Del, 21, essential, KQLKLIFQLQHQEKKPCLTKN, (SEQ ID NO: 4352); XM_014106867.1, MYBL2, A_7, In, 21, essential, KACCPVPCHREQRQPVLPRLL, (SEQ ID NO: 4353); XM_845734.4, NAPG, A_7, Del, 21, essential, KIFIRRLKITQLVIRKQLLKS, (SEQ ID NO: 4354); XM_535898.5, NUP153, A_7, In, 21, essential, TSIQLVCLWNAFPFTWEFFSP, (SEQ ID NO: 4355); XM_014112334.1, PRC1, A_7, Del, 21, essential, KHPRLAGTEPTRRTWSSMAAS, (SEQ ID NO: 4356); XM_005618818.2, SPRTN, A_7, In, 21, essential, NSSSVPYSYCQSPKCLKQLLS, (SEQ ID NO: 4357); XM_014112420.1, TICRR, A_7, Del, 21, essential, KTGQLNLPSLHGFQFCHLMPL, (SEQ ID NO: 4358); XM_545078.5, ZBTB11, A_7, Del, 21, essential, KEEQSRTWNGCVNNVEGNSLS, (SEQ ID NO: 4359); XM_005641516.2, ATRX, A_7, In, 22, driver, essential, high expression, KEEEGYSHAAKGHHTCRTSSNT, (SEQ ID NO: 4360); XM_014116355.1, BPTF, A_7, Del, 22, essential, high expression, KTNHIFDMNLLDMTGVGGNTGS, (SEQ ID NO: 4361); XM_846664.4, SEC62, A_7, Del, 22, essential, high expression, KKKRKERPKVEKKKIKRAGKKI, (SEQ ID NO: 4362); XM_014122300.1, GTPBP4, A_7, In, 22, essential, high expression, KEAENSAVQGEEHTGPQDASDG, (SEQ ID NO: 4363); XM_005630473.2, IMMT, A_7, In, 22, essential, high expression, RRGSWGQISYNCCRGETSQHDS, (SEQ ID NO: 4364); XM_014114888.1, FUBP1, A_7, Del, 22, driver, high expression, KYKMMLVFEFSLSQMMEQHLIE, (SEQ ID NO: 4365); XM_844507.4, AIDA, A_7, Del, 22, high expression, KGSPAPSALLSWRWMKLKPGRL, (SEQ ID NO: 4366); XM_005615655.2, AKAP7, A_7, Del, 22, high expression, KSTPNSMKNLSVIHLEKKWYIA, (SEQ ID NO: 4367); XM_014109908.1, ANK2, A_7, Del, 22, high expression, NPKSRTAMQASSVLPEQATWTK, (SEQ ID NO: 4368); XM_542164.5, CREB3L3, A_7, In, 22, high expression, NPPENQKQAVGPRKPEKEEGIY, (SEQ ID NO: 4369); XM_014119556.1, EEA1, A_7, In, 22, high expression, KPQLTGTSNSTNREAEESVRKP, (SEQ ID NO: 4370); XM_005640463.2, GLS, A_7, In, 22, high expression, KVFSRRHRHGWYIRLLFPAMLH, (SEQ ID NO: 4371); XM_014110232.1, GOLGB1, A_7, In, 22, high expression, KRCGNPPTNYPGEGSASDRNQL, (SEQ ID NO: 4372); XM_014109424.1, MGA, A_7, In, 22, high expression, RGTETTVPITVPIISAAGLMSV, (SEQ ID NO: 4373); XM_005639041.1, PPP3CA, A_7, Del, 22, high expression, KICWILMHRSQFVGTSMDNSLI, (SEQ ID NO: 4374); XM_014116985.1, SETX, A_7, Del, 22, high expression, KSSLNLKKNVKTRRILWETVEI, (SEQ ID NO: 4375); NM_001003154.2, TRDN, A_7, In, 22, high expression, TGKERKIRGTSKVVKEGTFSSK, (SEQ ID NO: 4376); XM_005621312.2, AGL, A_7, In, 22, high expression, KAAWSPRHENFGSRRYGLLWNL, (SEQ ID NO: 4377); XM_014115836.1, AKAP6, A_7, In, 22, high expression, KLLLLPKSRHQEWSGCGGLVWL, (SEQ ID NO: 4378); XM_547299.4, CLCA1, A_7, Del, 22, high expression, KTTIKMPQTHKTEDAISEAHGK, (SEQ ID NO: 4379); XM_532655.4, NTN4, A_7, In, 22, high expression, SLKIYQIENFTRKKNIVSRIMD, (SEQ ID NO: 4380); XM_014116024.1, PCNX, A_7, In, 22, high expression, TGYRIRCFNDHHCWSEVATILF, (SEQ ID NO: 4381); XM_005639205.2, PDLIM5, A_7, In, 22, high expression, NKPHPGDFSAVGTISSFSTEHA, (SEQ ID NO: 4382); XM_863229.4, TAOK1, A_7, In, 22, high expression, RKTRVAFKAEGEYTAFSPRGRG, (SEQ ID NO: 4383); XM_005624831.2, TEFM, A_7, In, 22, essential, KKVPGQPALEKAHQTRNRKREA, (SEQ ID NO: 4384); XM_531679.5, ZFC3H1, A_7, In, 22, essential, KSSERRGFLSYVQILHLTKSRR, (SEQ ID NO: 4385); XM_005635238.2, NELFCD, A_7, Del, 22, essential, KISLSLLRWCATGSTRTCLPRP, (SEQ ID NO: 4386); XM_859339.4, CTCF, A_7, In, 23, driver, essential, high expression, NKENQKEQTALHRGRQRCGCVCL, (SEQ ID NO: 4387); XM_005636161.2, RSRC2, A_7, Del, 23, essential, high expression, KGKCFGKAKKKGINPNLLKYGKN, (SEQ ID NO: 4388); XM_014115688.1, FERMT2, A_7, In, 23, essential, high expression, IQEQADNSKNLGGPSECSSDESN, (SEQ ID NO: 4389); XM_846266.3, PHAX, A_7, Del, 23, essential, high expression, KWDQRKRKMGKVISNGNDLSKTD, (SEQ ID NO: 4390); XM_014109171.1, PRKDC, A_7, Del, 23, essential, high expression, NDVGHEDFHLRHHCVYWIWSSGC, (SEQ ID NO: 4391); XM_005623082.2, ANKRD12, A_7, Del, 23, high expression, KLKKSANCCVVLFLKHLSIMMNM, (SEQ ID NO: 4392); XM_014106427.1, EPSTI1, A_7, Del, 23, high expression, KDFKKTSEEKHLESTTNTKLLSS, (SEQ ID NO: 4393); XM_531770.5, GCC2, A_7, In, 23, high expression, KHQNQAAACENQKRTGRFKASRN, (SEQ ID NO: 4394); XM_014122790.1, NUCB2, A_7, Del, 23, high expression, KRNSWSRIAGRQWISNSSSQRKN, (SEQ ID NO: 4395); XM_005627518.2, PHF3, A_7, In, 23, high expression, MAASSSSKDGTTSFTSEILRRKK, (SEQ ID NO: 4396); XM_535893.4, RANBP9, A_7, In, 23, high expression, NVEGCIQFTRLFGSLEQPSWKSA, (SEQ ID NO: 4397); XM_014115665.1, ZBTB1, A_7, In, 23, high expression, KFCVQIIYSKRTCVTALWTEFYL, (SEQ ID NO: 4398); XM_014118132.1, ZNF484, A_7, Del, 23, high expression, KSILERNTLNVLNVEKPSQGNQL, (SEQ ID NO: 4399); XM_005630596.1, APOB, A_7, Del, 23, high expression, KSLTDSLWTFRTRKSLRSLSLAT, (SEQ ID NO: 4400); XM_014112733.1, ARID4B, A_7, Del, 23, high expression, KQKKMKIWRIKMRRQLVWMNPSA, (SEQ ID NO: 4401); XM_005639497.2, KIAA2018, A_7, Del, 23, high expression, KISLFIPMGVSLVETAREQLFRG, (SEQ ID NO: 4402); XM_014112996.1, UIMC1, A_7, Del, 23, high expression, KTTVKADSSVSWSSLNTRLQMQK, (SEQ ID NO: 4403); XM_005632905.2, DNMT1, A_7, In, 23, essential, NAPGEEKETEQESDLLGWRCCQD, (SEQ ID NO: 4404); XM_536334.4, HEATR1, A_7, In, 23, essential, TRAFSSVYFSFYKWREVSVFSRF, (SEQ ID NO: 4405); XM_005627636.2, MDN1, A_7, Del, 23, essential, KVSCRPGDWSSELIFWKMPIQMS, (SEQ ID NO: 4406); XM_005624905.2, NSRP1, A_7, Del, 23, essential, KERKIILNCFWEKTESPSIFTTY, (SEQ ID NO: 4407); XM_547651.5, THOC1, A_7, In, 23, essential, KCCYLEIKYILFCWEKLFTTYVQ, (SEQ ID NO: 4408); XM_003638871.2, PTCH1, A_7, In, 23, driver, KLRQVLGCGPPHIWGLRCGIKGS, (SEQ ID NO: 4409); XM_535474.5, USP8, A_7, In, 24, driver, essential, high expression, KARFQATAGLLSFNTWTRKHQKSY, (SEQ ID NO: 4410); XM_532156.6, CDC5L, A_7, Del, 24, essential, high expression, KSLHLVSTILLRKTTRLLMQISEN, (SEQ ID NO: 4411); XM_533359.5, PRPF40A, A_7, In, 24, essential, high expression, RRGGKPTSKKNLYLEYKGGSKASI, (SEQ ID NO: 4412); XM_014106743.1, XRN1, A_7, In, 24, essential, high expression, NTKGMRTRRNHISPPTLVFNERVY, (SEQ ID NO: 4413); XM_843366.4, CHD1, A_7, Del, 24, essential, high expression, KKLRDFLVQEGQRGEKQELRRIKQ, (SEQ ID NO: 4414); XM_014120239.1, SOS1, A_7, Del, 24, essential, high expression, KLQETMDQVIILHFRVHLPQSSGI, (SEQ ID NO: 4415); XM_005626529.2, AFF4, A_7, Del, 24, driver, high expression, KASMDQNTPNHALPALENPRLFLH, (SEQ ID NO: 4416); XM_014121964.1, ARHGAP12, A_7, In, 24, high expression, KPNDLSEYSNCFWSHSVKARERDW, (SEQ ID NO: 4417); XM_534306.5, EIF2A, A_7, Del, 24, high expression, KISWRKFKKRKPFSRSWKIWNWVF, (SEQ ID NO: 4418); XM_005622049.2, USP33, A_7, Del, 24, high expression, NSIRNTEVLFQTFLMEQLLVQCSV, (SEQ ID NO: 4420); XM_005628768.2, AASS, A_7, Del, 24, high expression, KGSWFLDLVMYLNPFWNTCQEITG, (SEQ ID NO: 4421); XM_532364.5, IREB2, A_7, In, 24, high expression, IRFRELQRLGCQRTVSAGRESCFG, (SEQ ID NO: 4422); XM_542589.4, OLFM4, A_7, In, 24, high expression, KALQPNHPHRDHGKGYHFLHGTGL, (SEQ ID NO: 4423); XM_858916.4, MED1, A_7, Del, 24, essential, KILMNFLSTLRVLLIFITFQGTTN, (SEQ ID NO: 4424); XM_014118170.1, PNISR, A_7, Del, 24, essential, KDIVGVDLQQLKLDGVEVEVTHAE, (SEQ ID NO: 4425); XM_005627395.2, XPOS, A_7, Del, 24, essential, KLSQCWRQRCWTTRRAAWPPSLNP, (SEQ ID NO: 4426); XM_005629880.1, NRG1, A_7, Del, 24, driver, KSQGSQNFALAKHHWLILENICAK, (SEQ ID NO: 4427); XM_545261.4, NMD3, A_7, In, 25, essential, high expression, NFLLSGTVDFEIWNASEYTSYQRDS, (SEQ ID NO: 4428); XM_545545.5, PRKRA, A_7, Del, 25, essential, high expression, NRPRGTLLRNFLPNSVIFLQRTTFL, (SEQ ID NO: 4429); XM_005635616.2, CLCN3, A_7, In, 25, high expression, KGGAISCLSCRGFCSFWCTNWRSSF, (SEQ ID NO: 4430); XM_014109749.1, ITSN1, A_7, Del, 25, high expression, NLKLRRLSPATQLLVPSSSPWPLVS, (SEQ ID NO: 4431); XM_005622426.1, RGL1, A_7, Del, 25, high expression, KSSPSLPRRVPLSIPWTPLPQGCRP, (SEQ ID NO: 4433); XM_005618779.2, RYR2, A_7, In, 25, high expression, RQLPVGCTELYRREVSDVETRSRLH, (SEQ ID NO: 4434); XM_005623485.2, SYNE2, A_7, Del, 25, high expression, KLTMNYTDYKHFSSICSVITEIRIS, (SEQ ID NO: 4435); XM_014115665.1, ZBTB1, A_7, Del, 25, high expression, KS SYLNILPVLFDVLIVASGLKPKI, (SEQ ID NO: 4436); XM_005620894.2, DYNC1LI2, A_7, Del, 25, high expression, KQVLLEVLVLVVCRAQPRSQDKRLC, (SEQ ID NO: 4437); XM_005639497.2, KIAA2018, A_7, In, 25, high expression, KYHCLFQWESAWWKQPGNSCSGDNL, (SEQ ID NO: 4438); XM_005639497.2, KIAA2018, A_7, Del, 25, high expression, NSTERKTERHTMQWRGIERRKSMLE, (SEQ ID NO: 4439); XM_014106295.1, MYCBP2, A_7, In, 25, high expression, NIFTTRASEKTSKDPWQPCSSSCTF, (SEQ ID NO: 4440); XM_857524.4, TAOK3, A_7, Del, 25, high expression, KCPIVGNRHMRNGKIFSRKSSFYNN, (SEQ ID NO: 4441); XM_005620305.1, KMT2A, A_7, In, 25, driver, essential, KQLGPRPNCPIPGEGENPLPFHSFI, (SEQ ID NO: 4442); XM_005632905.2, DNMT1, A_7, In, 25, essential, NGSNQNSSVLQDPSSTVCAVWTVPG, (SEQ ID NO: 4443); XM_005628489.1, GTPBP10, A_7, In, 25, essential, KHDSKEDHGVPTYHPHLCNYRRRNR, (SEQ ID NO: 4444); XM_545951.5, RHOH, A_7, In, 25, driver, TGPGSESKGLSGVLGPQQPGGSAGV, (SEQ ID NO: 4445); XM_536371.4, TET1, A_7, In, 25, driver, KKTFYSARTSFWKEEGSNDDRSSRT, (SEQ ID NO: 4446); XM_005637236.2, CHD4, A_7, In, 26, driver, essential, high expression, KGAGGQLWGGAGVCGGGGRGGSSLRQ, (SEQ ID NO: 4447); XM_005617790.2, SRRM1, A_7, In, 26, essential, high expression, KGGHEQSKFGGYKALDNKTSNGNPWV, (SEQ ID NO: 4448); XM_014122663.1, UPF2, A_7, Del, 26, essential, high expression, KRNMKRKRKRSKKSRQNVSKKKKLPS, (SEQ ID NO: 4449); XM_005626529.2, AFF4, A_7, In, 26, driver, high expression, KPAWIRTLQITLFQPWKTPGCFFIEL, (SEQ ID NO: 4450); XM_005616994.2, ARHGAP21, A_7, Del, 26, high expression, KSPLLQERSVSDWMIVHQHIPIGIFH, (SEQ ID NO: 4451); XM_005637210.2, C1S, A_7, In, 26, high expression, KGLETSLPWRSNPLFQRSHCQFCLGA, (SEQ ID NO: 4452); XM_014106605.1, GOLGA4, A_7, Del, 26, high expression, KLQRRKMIYKEQPKDMKKSLMLVKKK, (SEQ ID NO: 4453); XM_014118119.1, PHIP, A_7, In, 26, high expression, KSWRQKTKKEDEDTKTRFRSHSSYKC, (SEQ ID NO: 4454); XM_005639910.2, SKIL, A_7, In, 26, high expression, NDDRHSRKRENDDQQQDANREEGTLG, (SEQ ID NO: 4455); XM_005623486.2, SYNE2, A_7, Del, 26, high expression, KIKSCAHGWCRWKTKFCRRQILVLKK, (SEQ ID NO: 4456); XM_544109.5, VCPIP1, A_7, In, 26, high expression, IAGNGFFHPSFDGQAFAGSKYRAVTI, (SEQ ID NO: 4458); XM_014115836.1, AKAP6, A_7, Del, 26, high expression, KVTPPPQVTSPGMVRLWRPGMALMNT, (SEQ ID NO: 4459); XM_014111650.1, GPRASP1, A_7, Del, 26, high expression, KNTGFGLEKRLITCQGPNPRKRPGPE, (SEQ ID NO: 4460); XM_005622209.2, KDM5B, A_7, In, 26, high expression, NADPQKEENQTEPLQGHEQFQVREGA, (SEQ ID NO: 4461); XM_014107325.1, BRCA2, A_7, In, 26, driver, essential, TKSFRYKRKSLAYSKSPSCATFRSGR, (SEQ ID NO: 4462); XM_005638256.1, CASC5, A_7, Del, 26, driver, essential, KSGKVRLGVVILHKIRRFLITMLKGI, (SEQ ID NO: 4463); XM_536013.5, GTF3C3, A_7, Del, 26, essential, KMQKKELQKRIKLVRMLPAVYLMVCQ, (SEQ ID NO: 4464); XM_014120363.1, PTCD3, A_7, In, 26, essential, KLLFLLHNDPRNGEAPSSCAGIKLVC, (SEQ ID NO: 4465); XM_005625106.2, SEC16A, A_7, Del, 26, essential, KGQKLICQTTRTNRSSGMKRRTGGWM, (SEQ ID NO: 4466); XM_547651.5, THOC1, A_7, Del, 26, essential, KMLLLGNQIHSILLGKIIYYVCAMIS, (SEQ ID NO: 4467); XM_005641567.1, BTK, A_7, Del, 26, driver, KILPLKDRFQLYMMKGLFMSSPQLKN, (SEQ ID NO: 4468); XM_005641516.2, ATRX, A_7, Del, 27, driver, essential, high expression, KRRRGILPCCQRTPYLQNFFKYIKNTL, (SEQ ID NO: 4469); NM_001287118.1, CFDP1, A_7, In, 27, essential, high expression, KKGSEHSGQEEKTRWLLIRRRGRGGCQ, (SEQ ID NO: 4470); XM_005621408.2, RBBP6, A_7, Del, 27, essential, high expression, KSSSTEKLVKKLEVQKMYLIQKNPLKN, (SEQ ID NO: 4471); XM_860109.5, RBM39, A_7, In, 27, essential, high expression, KQSCSNGKQSTKGKCWTYEALCGLITL, (SEQ ID NO: 4472); XM_845208.4, HECTD1, A_7, Del, 27, essential, high expression, KSQQKFCSRLRNHWPWQVELYQTGVNS, (SEQ ID NO: 4473); XM_847262.4, MRPS23, A_7, Del, 27, essential, high expression, KSSLCKKFHRTRIRRLLKNSRKVSHLP, (SEQ ID NO: 4474); XM_014120373.1, ALMS1, A_7, Del, 27, high expression, KRVGHYQKLLISPHVERSTVFSTNRSF, (SEQ ID NO: 4476); XM_014120373.1, ALMS1, A_7, Del, 27, high expression, KVRCFLLIKLENPIRLKLNRLSLISIS, (SEQ ID NO: 4477); XM_014109907.1, ANK2, A_7, Del, 27, high expression, KINRMSRNGSRRGWHILLITLASAGQN, (SEQ ID NO: 4478); XM_005635616.2, CLCN3, A_7, Del, 27, high expression, NKKVLWAVLVCVLHSTPRLFQQKVHGH, (SEQ ID NO: 4479); XM_005618961.2, DDX50, A_7, In, 27, high expression, KPLTKGSCFGSYKGTGKPSSQRLQRYH, (SEQ ID NO: 4480); XM_014120322.1, KIDINS220, A_7, In, 27, high expression, NVLPTIFCHLPFYLWLHYCGNYPPGYL, (SEQ ID NO: 4481); XM_005623784.2, PAPOLA, A_7, Del, 27, high expression, KQKTLKISVLISPMIFSLSQIQFIGKQ, (SEQ ID NO: 4482); XM_005615470.1, PHACTR2, A_7, In, 27, high expression, SNWVQIISFTIYFLHLISSQSFEGDTL, (SEQ ID NO: 4483); XM_014118118.1, PHIP, A_7, In, 27, high expression, KDVKTPAKIRYLYLSILYTYTNSSTKA, (SEQ ID NO: 4484); XM_535242.6, PLK2, A_7, Del, 27, high expression, KQFTIMQSLASALFSQQQMLLNNSLVK, (SEQ ID NO: 4485); XM_005634561.2, RNF13, A_7, In, 27, high expression, NLSGLQAKSGSFPRRLRLRHRQQSRRK, (SEQ ID NO: 4487); XM_855384.3, USP15, A_7, Del, 27, high expression, KDILMKMPQRILKNMKVWNINLLKNPL, (SEQ ID NO: 4488); XM_535304.5, CHD9, A_7, Del, 27, high expression, KGGEGGRMSKVLTSSFLTEINHLIMLL, (SEQ ID NO: 4489); XM_005620651.1, HSBP1, A_7, In, 27, high expression, KHCRPHDPGWGGRAGGGEQDPCHTEEL, (SEQ ID NO: 4490); XM_014110285.1, PARP9, A_7, In, 27, high expression, KEESDGRENTWETCELQVVSASSLPVL, (SEQ ID NO: 4491); XM_531828.5, RTN4, A_7, Del, 27, high expression, KMRLLMSRMKLDHYLAQSYPMTFLSRL, (SEQ ID NO: 4493); XM_005620921.2, SUMF2, A_7, Del, 27, high expression, KSTGQRLRCSGGVLSLRSLSPMSLETK, (SEQ ID NO: 4494); XM_014112442.1, BLM, A_7, In, 27, driver, essential, TESCFSGKDVSERRSGKEVSWRTDRSV, (SEQ ID NO: 4495); XM_014108433.1, ATF7, A_7, Del, 27, essential, KRLLLGPLTCLCLLHQTSKSKKKSQWR, (SEQ ID NO: 4498); XM_014117059.1, MAPKAP1, A_7, Del, 27, essential, KSPLKRNRQVLESSQYYLYDWSSALCS, (SEQ ID NO: 4499); XM_014111598.1, MBTPS2, A_7, In, 27, essential, KFKFKFLYNTFFGNSHSLNKSETSTSN, (SEQ ID NO: 4500); XM_005629238.2, METAP2, A_7, Del, 28, essential, high expression, KDGRRRRVKGLPQGNKNLIKNQEPQLMR, (SEQ ID NO: 4501); XM_533359.5, PRPF40A, A_7, Del, 28, essential, high expression, KKRRKANQQKKPILGIQRRKQSKHLKNY, (SEQ ID NO: 4502); XM_005630347.2, CEBPZ, A_7, Del, 28, essential, high expression, KEQLMTFNKVNWKHLFRILVWPSMQKLS, (SEQ ID NO: 4503); XM_536323.5, HMGCR, A_7, Del, 28, essential, high expression, KSQMIVVDVNLCWSGIATKSMQQRRRQG, (SEQ ID NO: 4504); XM_014113675.1, PTPRK, A_7, Del, 28, driver, high expression, KGGATILTPTTSSLLKNAKMPWGTPGRR, (SEQ ID NO: 4506); XM_005626595.2, FAM13B, A_7, Del, 28, high expression, KLDNLRNSLKGKEIASPPTVILQPIQRY, (SEQ ID NO: 4507); XM_005640463.2, GLS, A_7, Del, 28, high expression, NLILEEKVVIKGIPLDHWTMKVSNKNLL, (SEQ ID NO: 4508); XM_535945.5, NOSTRIN, A_7, In, 28, high expression, KLSRQCLGLGLRGHEVSGGPTSKTWQSN, (SEQ ID NO: 4509); XM_005631042.2, RTN3, A_7, Del, 28, high expression, KALRQHKFSLIFLKGVQLVRPHVHKYLT, (SEQ ID NO: 4510); XM_005630385.2, NCAPH, A_7, In, 28, essential, NKGCYYSDQVHFGEPELESHHSSYRFPL, (SEQ ID NO: 4512); XM_005615806.2, NAA35, A_7, In, 28, essential, NKEKEESSPIEPRDHNEPGISEHVCWNV, (SEQ ID NO: 4513); XM_014116492.1, NF1, A_7, In, 28, driver, KFISLSVDYIGYTGKMSCWATKGHDEIR, (SEQ ID NO: 4514); XM_005641516.2, ATRX, A_7, Del, 29, driver, essential, high expression, KEFQIKKTMILLKMKNTEKKEWIIKGRKI, (SEQ ID NO: 4515); XM_861061.4, METAP2, A_7, Del, 29, essential, high expression, KDGRRRRVKGLPQQGNKNLIKNQEPQLMR, (SEQ ID NO: 4516); XM_005624952.2, PAFAH1B1, A_7, Del, 29, essential, high expression, KVASLGHSYCLDPETRLLRCGTSVLACAL, (SEQ ID NO: 4517); XM_014116402.1, DDX42, A_7, Del, 29, essential, high expression, KSLILFLQLIIQRLTIHHLKKIFTMSMKR, (SEQ ID NO: 4518); XM_005621532.2, GSPT1, A_7, In, 29, essential, high expression, RTCKCGIHWACRCWQVNHWRTNNVFDRNG, (SEQ ID NO: 4519); XM_014110816.1, USP48, A_7, In, 29, essential, high expression, KSRCAEHCPTAVLWRICLRNRLQSMWQRV, (SEQ ID NO: 4520); XM_005627838.2, UBRS, A_7, Del, 29, driver, high expression, KRKEKNRMWCQKKLKVQNQGHLLMILLHN, (SEQ ID NO: 4521); XM_014110851.1, EIF4G3, A_7, Del, 29, high expression, KNSLLKSYISDSRNSLLRTRRMMNRSLTG, (SEQ ID NO: 4522); XM_014108479.1, KIF21A, A_7, Del, 29, high expression, KVWPVKRIIQTLTKRRKKKRVLQKEKTMN, (SEQ ID NO: 4523); XM_014119322.1, MACF1, A_7, Del, 29, high expression, KLQTDNPGSRTVCKKLRNISGMFKTSCHG, (SEQ ID NO: 4524); XM_014109424.1, MGA, A_7, Del, 29, high expression, KRNRDNRPHHCPHHFSSRAHVSLILRTVV, (SEQ ID NO: 4525); XM_014107673.1, TRAF3IP1, A_7, Del, 29, high expression, KFWRPRKIMRSYSGHPDLERRAATGTNLV, (SEQ ID NO: 4526); XM_533405.4, FAM210A, A_7, Del, 29, essential, KRKNLILYKTNLLIYINDLRKHLDNMGKF, (SEQ ID NO: 4528); XM_014107008.1, DIDO1, A_7, Del, 30, essential, high expression, NKSCRLLRRKSQNPSMRPLPLSQFLAPLMK, (SEQ ID NO: 4529); XM_014122663.1, UPF2, A_7, In, 30, essential, high expression, RRQGTEEKRRRKGEGGGRIKEKRGRRKKET, (SEQ ID NO: 4530); XM_005619310.2, CSNK1A1, A_7, Del, 30, essential, high expression, NKNMKRLVKRRCPLLLKFYVRGFLQNLPCT, (SEQ ID NO: 4531); XM_846524.3, ACSL3, A_7, In, 30, driver, high expression, RKQRGYNYVETNTDGSCSGNHGSDLQKCHE, (SEQ ID NO: 4532); XM_005640279.2, NOSTRIN, A_7, In, 30, high expression, KPSWLCCQTEVGKYPGKLLPEYPGAGEGKN, (SEQ ID NO: 4535); XM_534296.5, PCOLCE2, A_7, In, 30, high expression, IAYNYSTTCYHHVPCNYRFKTHRFPVSTKV, (SEQ ID NO: 4536); XM_005623486.2, SYNE2, A_7, Del, 30, high expression, NRISPILTIMKILSYLKKKNQLLIFQQICP, (SEQ ID NO: 4537); XM_532445.5, WASL, A_7, In, 30, high expression, SGTKQSTGILLWKGCTFRPDTTGYSAEICI, (SEQ ID NO: 4538); XM_014106506.1, EPB41, A_7, Del, 30, high expression, KRQRVVKNMLHQESHLESKMDHFLTLILPW, (SEQ ID NO: 4539); XM_014107556.1, FDFT1, A_7, Del, 30, high expression, KRFHCYTTFTLTFMNQTGGIWRARRRIDKC, (SEQ ID NO: 4540); XM_014117291.1, GOLM1, A_7, Del, 30, high expression, KRTKASSSEPPVSLSPGHRKQTCLRQRYHK, (SEQ ID NO: 4541); XM_854556.3, NRD1, A_7, Del, 30, high expression, NAGLLRYLVEMVRQDLSKILLTQYSAFQLH, (SEQ ID NO: 4542); XM_014116764.1, ATAD5, A_7, Del, 30, essential, KSWKILIFKWFQIHSHILIKEVFLRRKVKS, (SEQ ID NO: 4543); XM_537272.5, HAUS1, A_7, In, 30, essential, KFDCNVSVRKMSTRGSKEGRAASLYRKGQS, (SEQ ID NO: 4544); NM_001003294.1, DLD, A_7, In, 31, essential, high expression, SSRKDGCHWCRSNRCRIGFSLAETWCRCDSS, (SEQ ID NO: 4546); XM_846266.3, PHAX, A_7, In, 31, essential, high expression, NGIKGRGKWARSSQTETTCQRQTRGQTRNEL, (SEQ ID NO: 4547); XM_531814.5, MSH6, A_7, Del, 31, driver, high expression, NEGSYQTLRVTLVALMWNLSQMLRRKEAVMK, (SEQ ID NO: 4549); XM_005637209.2, C1R, A_7, Del, 31, high expression, KLWGGSVGSWVLHWATPQKQGNSCLKGTRCS, (SEQ ID NO: 4551); XM_540029.5, TRAPPC11, A_7, Del, 31, essential, KSEVQSCLLSMLHGCLNNSRPLEIYLMKLLS, (SEQ ID NO: 4553); XM_005629808.2, KAT6A, A_7, Del, 31, driver, NQFFTEEGGSENANTTIAVWSQKLFQRLQRC, (SEQ ID NO: 4554); XM_005619169.2, NSD1, A_7, Del, 31, driver, KMEMALRNQYVLALVGVTLHCLENYLYLYLA, (SEQ ID NO: 4555); XM_003639390.3, TLN1, A_7, In, 32, essential, high expression, KQGSFWAGGRRGVYYAGGLSFPQKVDSPPAAV, (SEQ ID NO: 4557); XM_538778.5, RAD23B, A_7, In, 32, essential, high expression, KLCGGYGDKTQSSDNTSTSYNPAVESCHHYYS, (SEQ ID NO: 4558); XM_005627613.2, RARS2, A_7, Del, 32, essential, high expression, KSIFSKCSKCFRSWDMTGQKGASTCPLEWCRE, (SEQ ID NO: 4559); XM_850902.4, G3BP2, A_7, Del, 32, high expression, KQELQESEKPEVVVMIAGILGAMIEVLVVHVA, (SEQ ID NO: 4560); XM_014112496.1, KIAA0232, A_7, In, 32, high expression, IWHGEECSDVPGFSGGISWDSAGGEAESVFGM, (SEQ ID NO: 4561); XM_014119322.1, MACF1, A_7, In, 32, high expression, NCRQTIQAQGLYAKSSEISVACSRPRAMDRRL, (SEQ ID NO: 4562); XM_850281.4, POLE3, A_7, Del, 32, high expression, KPMQRSKTRAEMRTTMKTKNGWKKKNRTKRRK, (SEQ ID NO: 4563); XM_014118865.1, UTRN, A_7, Del, 32, high expression, KRPLMKSLRIRDLHYIHLQKKQRLWRKLFLQM, (SEQ ID NO: 4565); XM_014114846.1, ZNF644, A_7, Del, 32, high expression, NTWNTCIHHHVLIHLVVLLDLINEKVTFSKNL, (SEQ ID NO: 4566); XM_014119970.1, GIMAP4, A_7, In, 32, high expression, NRSVTRILQSRARETESADKRGVWRENQNAGG, (SEQ ID NO: 4567); XM_863229.4, TAOK1, A_7, Del, 32, high expression, KKNKSGFQSRRRIYSISKQRKRLIFFDDKGNT, (SEQ ID NO: 4568); XM_537313.5, NDC80, A_7, In, 32, essential, NGVRRYFRTVKYNDNRKQERCKNSERRSSKAG, (SEQ ID NO: 4569); XM_005637237.2, CHD4, A_7, In, 33, driver, essential, high expression, KGAYALMPAAGGQLWGGAGVCGGGGRGGSSLRQ, (SEQ ID NO: 4574); XM_014115260.1, AHCTF1, A_7, In, 33, essential, high expression, NRDSSPAARISFRFIFSVCLLTSCFKDQAENHI, (SEQ ID NO: 4576); XM_547275.5, DNTTIP2, A_7, Del, 33, essential, high expression, KTTVSHHIANQSINFRKNAEKNDRKQQVMAGLA, (SEQ ID NO: 4577); XM_014107587.1, NCL, A_7, Del, 33, essential, high expression, KSQLPHQPRKQLSPLAKRQQLHQPKRRLHQPKQ, (SEQ ID NO: 4578); XM_531939.5, PSIP1, A_7, Del, 33, driver, high expression, KYGDSKLVRLSWKSLQCCITSLRICFWLVKEIL, (SEQ ID NO: 4579); XM_005635616.2, CLCN3, A_7, In, 33, high expression, TRRYCGQFSCVFCTAHPVSSSRKSTATEASEHS, (SEQ ID NO: 4580); XM_005615470.1, PHACTR2, A_7, Del, 33, high expression, KQLGPNHQLHHLLPPPHLVPELRRRHSLAKQGQ, (SEQ ID NO: 4582); XM_014108414.1, PLEKHA5, A_7, Del, 33, high expression, KFTILERDLIQLKGILMHLWSDEVGFINRTVLA, (SEQ ID NO: 4583); XM_846233.4, PPFIA1, A_7, Del, 33, high expression, KIVNFKKSMNCSRKPGDKVCLLHSGMGRPLLFG, (SEQ ID NO: 4584); XM_544109.5, VCPIP1, A_7, Del, 33, high expression, NCRKWFLPSKLRWTSICGIKVQSSHHLIFLKEK, (SEQ ID NO: 4585); XM_014109945.1, WDFY3, A_7, Del, 33, high expression, KWCEMICFITITPMCQKRSKRQMWRNPLDTEEP, (SEQ ID NO: 4586); XM_014121014.1, AHNAK, A_7, In, 33, high expression, RYQPRGSQNGHREPKNQCGRCRRKLKRSQTQDA, (SEQ ID NO: 4587); XM_005620894.2, DYNC1LI2, A_7, In, 33, high expression, NRFSWKSWCWWCAEHSQEVRTKDCVDKCSGRTG, (SEQ ID NO: 4589); XM_859741.4, WAPL, A_7, Del, 33, essential, KELKQLHPPPCSLLLKALIIPRTVSLVLTMQKT, (SEQ ID NO: 4590); XM_005632616.2, SETD2, A_7, Del, 34, driver, essential, high expression, KGDRNWRVILKVMVNFRTERKFEWRWSRGKRQCP, (SEQ ID NO: 4591); XM_005622377.2, CEP350, A_7, In, 34, essential, high expression, KVRATARKSLFTDRQFTNSLCEGHSQSTTRNQKI, (SEQ ID NO: 4592); XM_014112813.1, BICC1, A_7, In, 34, high expression, NFCFFKWTCTVSKYKIWCNTHLITWRKSAEWESW, (SEQ ID NO: 4593); XM_014112496.1, KIAA0232, A_7, Del, 34, high expression, NLAWRRVLRRPWILRRNQLGFCRWGSRISVWNVA, (SEQ ID NO: 4594); XM_014120322.1, KIDINS220, A_7, Del, 34, high expression, KRVAYHLLSSSFLSLAALLRELPSWLSLELTQNI, (SEQ ID NO: 4595); XM_014119574.1, OSBPL8, A_7, In, 34, high expression, RISQGAKEKLPRRKEKSHKGVTQYNHRSFCYCYG, (SEQ ID NO: 4596); NM_001038648.1, TMEM57, A_7, Del, 34, high expression, KGRSWKKLLLPGPLHLLLHLGGSAPKPYGIGSEN, (SEQ ID NO: 4597); XM_005620337.1, USP24, A_7, Del, 34, high expression, KTRRMDSSHLSLIIPSLCGWCQLCVSSTRSPARL, (SEQ ID NO: 4598); XM_005639670.2, CMBL, A_7, Del, 34, high expression, NTAKWNIKLKHFLGKPMASCIGREKIVQQKTSPI, (SEQ ID NO: 4599); XM_014108105.1, FGF1, A_7, In, 34, high expression, KLVCWSQEEWKLQTRSSDSLWSKSNFVSPPASVL, (SEQ ID NO: 4600); XM_005632616.2, SETD2, A_7, Del, 35, driver, essential, high expression, KQVLRRNPHNLKAPFLLQNLMKILYGLPQVKDHMI, (SEQ ID NO: 4601); XM_005619382.2, NIPBL, A_7, Del, 35, essential, high expression, KENKILTHRRLGVLQEVIDQLLRRRVLREMGQGQH, (SEQ ID NO: 4602); XM_005617866.2, HP1BP3, A_7, In, 35, high expression, NNTFLGYPFCQPASQGPETNTDGFLPTSQDGCNPN, (SEQ ID NO: 4603); XM_014108478.1, KIF21A, A_7, In, 35, high expression, NKGSTNETNERRTRKSQIDRVQEKQRDCSIEKRST, (SEQ ID NO: 4604); XM_014121888.1, MAP4, A_7, Del, 35, high expression, KQRHLLRLESLNLTQSLKQLVPLRVHRNHLLGKSR, (SEQ ID NO: 4605); XM_014116985.1, SETX, A_7, Del, 35, high expression, NVRENDLRNLWLKILYGLHHLREMRTSLIFLMGEV, (SEQ ID NO: 4606); XM_544645.5, UBR1, A_7, Del, 35, high expression, KGENKKTKMKHYHHHHLLNSALLSAKWLTFSIVIS, (SEQ ID NO: 4607); XM_005633672.2, USP47, A_7, Del, 35, high expression, KHGRILALSFWIIIFMKKILIFPATGRFSLKFLMG, (SEQ ID NO: 4608); XM_005619006.2, P4HA1, A_7, In, 35, high expression, MGREVRSTNQHSNKRSRRICWAPCKCIQINETSEH, (SEQ ID NO: 4609); XM_005620305.1, KMT2A, A_7, Del, 35, driver, essential, KTTWTSAQLPHPWRRRKPSAFPLLHLALLNIPLPP, (SEQ ID NO: 4610); XM_005636081.1, SFSWAP, A_7, Del, 35, essential, KRSRGSALMMMMMKKMEVTCTRLFLLPRSVIAWRS, (SEQ ID NO: 4611); NM_001003057.1, TCOF1, A_7, Del, 35, essential, KRKRRRRQKRPQPKTLTHYSRRKRRKRRRQQSKLS, (SEQ ID NO: 4612); NM_001313780.1, GMPS, A_7, Del, 36, driver, essential, high expression, KVLEKMESSVLVWTTLAHCSGAFRRKKLFCLPMEIV, (SEQ ID NO: 4613); XM_005635410.2, MRPS31, A_7, Del, 36, essential, high expression, KVLGKICSKGRDLIFLTLRQLLKRHLKQRLHLHSGI, (SEQ ID NO: 4614); XM_005637210.2, C1S, A_7, Del, 36, high expression, KRAGNFVTVEIQSLVPKKSLPILFGSLRKQYMYSKM, (SEQ ID NO: 4617); XM_535698.4, LARP7, A_7, Del, 36, high expression, KRNGHELNKCWQILLSKWTSGLEMQIYTKIDFFESK, (SEQ ID NO: 4618); XM_005622349.2, PPP2R5A, A_7, Del, 36, high expression, KLRSLFLSRYPSVYPVLIFRLQKGHCTSGIMSIFLV, (SEQ ID NO: 4620); XM_014119604.1, TMPO, A_7, In, 36, high expression, RTLHSHVDKNFAVCCCGHFFVFGLSSYGNQPRKSVQ, (SEQ ID NO: 4621); XM_014107674.1, TRAF3IP1, A_7, Del, 36, high expression, KFWRPRKIMRSYSGHPDLERRRNCLSLSQHGRRRRT, (SEQ ID NO: 4622); XM_535304.5, CHD9, A_7, Del, 36, high expression, KLINPGTIKMAINSGNINWKDSTGSYSIGTIDETAF, (SEQ ID NO: 4624); XM_014108105.1, FGF1, A_7, Del, 36, high expression, KIGLLVSRRMEAANAVLGLTMVKKQFCFSPCQCPLI, (SEQ ID NO: 4625); XM_005639155.2, PTPN13, A_7, Del, 36, driver, KQAFLMQQITLTVEIQTWMKLLIPAVRIIKHQKREV, (SEQ ID NO: 4626); XM_014113201.1, MLLT4, A_7, In, 37, driver, high expression, TRLLPAQPLERTKLLPGICRDSCWHPRCLSGPERKTV, (SEQ ID NO: 4628); XM_014106549.1, ZBTB38, A_7, Del, 37, high expression, KMKKGMKNLQSPMGQGSQMHFSPSKQKIVITCFLHWT, (SEQ ID NO: 4630); XM_005622209.2, KDM5B, A_7, Del, 37, high expression, KPPMLWICMSASCVAVATMKTGFSCVMGVTTVTTPFA, (SEQ ID NO: 4631); XM_014115880.1, MIS18BP1, A_7, Del, 37, essential, KTIKVKVALSGAISRKKQLKLIFQLQHQEKKPCLTKN, (SEQ ID NO: 4632); XM_014112334.1, PRC1, A_7, In, 37, essential, NTPGWQERSQQGEPGAQWQHPERWVPRLGSPPAQLQH, (SEQ ID NO: 4633); XM_014118218.1, REV3L, A_7, Del, 37, essential, KIFRRDFRKFSNRMISLSEHYQDQWTTAMDLRSSLLS, (SEQ ID NO: 4634); XM_005634924.1, ASXL1, A_7, In, 37, driver, NWGDAAASCPDSSEGKRGPRGIGVRSDEAQQRGRDRF, (SEQ ID NO: 4635); XM_014116355.1, BPTF, A_7, Del, 38, essential, high expression, KHSYRRIMTPLFLLPRALHFHQYLKVLMIEIPHLCQKQ, (SEQ ID NO: 4636); XM_014111626.1, THOC2, A_7, Del, 38, essential, high expression, KISQNLQAKKKVVIRLNLKRWIKSPPVAKRSPGMIKKR, (SEQ ID NO: 4637); XM_535836.5, KNG1, A_7, In, 38, high expression, NLPYCQLSVTGKGHIDEKASRFFTFPILIHGENRKRNN, (SEQ ID NO: 4639); XM_014106660.1, PIK3CB, A_7, Del, 38, high expression, KLASSSTLTLDIFLEISSLNLALKGNGCLLFLPMISFM, (SEQ ID NO: 4640); XM_538880.5, FKBPS, A_7, Del, 38, high expression, KRLRSTMSETAGYMPTCLRSLQNRMQRKRPVKPWARRL, (SEQ ID NO: 4641); XM_005622961.2, PRG4, A_7, In, 38, high expression, TKDYTNSPKDDYIDNAQITPYLFSGSHAPNYHQPQPKT, (SEQ ID NO: 4642); XM_533945.4, SAFB2, A_7, Del, 38, high expression, KSLKTLRRKKKMRMSRNQDLQIGPESPNPEAEEWNGRS, (SEQ ID NO: 4643); XM_014110821.1, DYNC1I2, A_7, In, 39, essential, high expression, NYPRICVSLPVCCDVCYICKISSKSGCWWYIFRPNCAVG, (SEQ ID NO: 4644); XM_843366.4, CHD1, A_7, Del, 39, essential, high expression, KRDRLIHLRRKMMKKIMIMIKEVLVAKQLSMLAIKKMKK, (SEQ ID NO: 4645); NM_001313804.1, ALCAM, A_7, In, 39, high expression, KHDCFNSYHSSLFGFVLKPKWRSDQADWRCLACVMHNIC, (SEQ ID NO: 4647); XM_845588.3, BRE, A_7, Del, 39, high expression, KIIRTAPDGMEMKWPKEQRLISKPLSLSSRRQRLPMESS, (SEQ ID NO: 4648); XM_014107158.1, BCAS1, A_7, Del, 39, high expression, KIWKTRHPKQKVSVMAKRDRRPVRTKLKAPRKSTCTAPG, (SEQ ID NO: 4649); XM_535304.5, CHD9, A_7, In, 39, high expression, RKDRESKFREWGRKLCVKFSFHILYCCIKYSCSCQPISS, (SEQ ID NO: 4650); XM_005624373.2, NMT1, A_7, In, 39, essential, KGHSSSAPAPHQVPEAVPPHASHEPGGGGALVLSPGEYH, (SEQ ID NO: 4652); XM_014109969.1, PTPN13, A_7, Del, 39, driver, KQAFLMQQITLTVEIQTWMKLLIPAVRIIKHQKRNLPPQ, (SEQ ID NO: 4653); XM_005641516.2, ATRX, A_7, Del, 40, driver, essential, high expression, KELENTVKKTQEMKEQKIKSIMNQIQILKNLRSQDTDIGF, (SEQ ID NO: 4654); XM_005631997.2, PRPF40A, A_7, Del, 40, essential, high expression, KSRLLMLTKSRQRKKKKKRQDQNTKRPRNHFNVFLKIMRK, (SEQ ID NO: 4655); XM_005621271.2, RNF40, A_7, Del, 40, essential, high expression, KLQMRMPCGAFGRRRSRSNICSASWVLPSRKRKLCCQRWM, (SEQ ID NO: 4656); XM_014109172.1, PRKDC, A_7, Del, 40, essential, high expression, KTTLLKSCSRISVIFLTPLSLSFHLLSPVFRKSVANTQTC, (SEQ ID NO: 4657); XM_014106956.1, ADNP, A_7, In, 40, high expression, KAHLQMHPLPWCIHQQHDRLDYHSASSSLQRCWKDPEWPG, (SEQ ID NO: 4658); XM_014110285.1, PARP9, A_7, Del, 40, high expression, KLPGYIKVLSWYWSQKDLNCPVNVYTMCCGTQDVSKSLRC, (SEQ ID NO: 4659); XM_014106331.1, BORA, A_7, In, 40, essential, IQLGKYNQPFTSFFTHFLTDRISNRKDSTPGTKEVYFSFS, (SEQ ID NO: 4660); XM_014115508.1, CEP192, A_7, Del, 40, essential, KQKLVDMKVDWKTFQELVRLIFGIYLCPKNRLHKTSIQWT, (SEQ ID NO: 4661); XM_859725.4, WAPL, A_7, Del, 40, essential, KELKQLHPPPCSLLLKALIIPRTVSLVLTMQRTCLVCLKV, (SEQ ID NO: 4662); XM_014120448.1, EML4, A_7, Del, 40, driver, KTTKKENMLKCLCEVGQLRCSFLLMLKTMMTSEQNCLLRN, (SEQ ID NO: 4663); XM_014115338.1, ASH1L, A_7, Del, 41, high expression, KSVQTSTWMSNPFLVTRPPPVTVRSQRMTPGRAVWMTASIE, (SEQ ID NO: 4664); XM_014113549.1, CHD3, A_7, In, 41, high expression, KGEEDKAAEKRGGRWGTKGRTEVVSHSASDLGPGGRGTCVL, (SEQ ID NO: 4665); XM_534241.4, TOP2B, A_7, Del, 41, high expression, KVKILGISSHFLHTLRSQKMIQLNLIAMKKILPLFFHRHLV, (SEQ ID NO: 4666); XM_014115665.1, ZBTB1, A_7, Del, 41, high expression, KVLCPNYLLQKNVCHGALDGVLPVTVVDLALAVKNYWMSMC, (SEQ ID NO: 4667); XM_014114460.1, ZNF326, A_7, Del, 41, high expression, KTVRNTEMDTEWHLRVHFVNSEHLKKKILNYIWKVLHTKKH, (SEQ ID NO: 4668); XM_859339.4, CTCF, A_7, Del, 42, driver, essential, high expression, KQRKPKRANCVTQRKAKMWMCLSMILRKNSRRACCQRLMQRK, (SEQ ID NO: 4670); XM_014122183.1, SMARCC1, A_7, Del, 42, essential, high expression, KIVRRSRTVKLVRILNQKRKLRRTRNSLILVKKEKAISGRRK, (SEQ ID NO: 4671); XM_003638878.3, KIF5B, A_7, Del, 42, driver, high expression, KIRPCGTPFSGLKMNSTGGVMERQCLLMNSLTKRKPTWKLLQ, (SEQ ID NO: 4672); XM_014113670.1, PTPRK, A_7, Del, 42, driver, high expression, KDIYVGEDTSALETLGGATILTPTTSSLLKNAKMPWGTPGRR, (SEQ ID NO: 4673); XM_014113564.1, CHD3, A_7, In, 42, high expression, KGEEDKAAEKRGGRWGTKAGRTEVVSHSASDLGPGGRGTCVL, (SEQ ID NO: 4674); XM_005636978.2, KIF21A, A_7, Del, 42, high expression, KGYRNLRKAIEKKEVWPVKRIIQTLTKRRKKKRVLQKEKTMN, (SEQ ID NO: 4675); XM_014119477.1, UTP20, A_7, In, 42, essential, KPSSPYSGPTPSTPELSSAAPNSSARWAESCCEQENQHAHIY, (SEQ ID NO: 4677); XM_014116492.1, NF1, A_7, Del, 42, driver, KRKTKNQWLASVSLRTASVQWEVPCSSDLSILPLSHRTKQGF, (SEQ ID NO: 4679); XM_536121.3, IARS2, A_7, In, 43, essential, high expression, SEIYSWISTECHGRNDGQTAILVYIKAKSLGCSNPCISSQDKR, (SEQ ID NO: 4681); XM_014122184.1, SMARCC1, A_7, Del, 43, essential, high expression, KIVRRSRTVKLVRILNQKKRKLRRTRNSLILVKKEKAISGRRK, (SEQ ID NO: 4682); XM_533429.5, TXLNB, A_7, Del, 43, high expression, KLETQKCLKGMTKVSTPPTKSPSLLCLGGQGLMPRKSVVFTML, (SEQ ID NO: 4684); XM_014106519.1, EPB41, A_7, Del, 43, high expression, KRQRVVKNMLHQESHLESKMDHFLTLMWVTSSPPSFEVSSLPW, (SEQ ID NO: 4685); XM_014108547.1, LRRK2, A_7, Del, 43, high expression, KTLCGEDVVQNFSHFLMISPFRNSLRQRQISCFLMQHSVIPTS, (SEQ ID NO: 4686); XM_014112349.1, BOD1L1, A_7, Del, 43, essential, KERKIQGMLKRILKRNCNLKKIPKKLSRQVSIVKRKRYLLQRI, (SEQ ID NO: 4687); XM_014112349.1, BOD1L1, A_7, Del, 43, essential, KTNQQTKVKRSQRAMTKEKERKKRRKRLKRNLITQKGVRNYKK, (SEQ ID NO: 4688); XM_005623362.2, PRPF39, A_7, Del, 43, essential, KTFPNQERCFWKQSKETKKIQSYTSIYLKWNIVVTSNKMKKIS, (SEQ ID NO: 4689); XM_014118218.1, REV3L, A_7, Del, 43, essential, KLVSNKGQIVKILKELCLLGENDHMLSCLLPHHLTTLKPKTVT, (SEQ ID NO: 4690); XM_548044.5, NSF, A_7, Del, 44, essential, high expression, KALTPTLTILTRWQQNSFSSSTTRHSQWDSSLCLASMKNFLACW, (SEQ ID NO: 4691); XM_863207.4, SNW1, A_7, Del, 44, essential, high expression, KCQMHWPFRWMLKEKLNMMQLLGKDSQKTRSFIASILTWFPRKS, (SEQ ID NO: 4692); XM_014110108.1, ABI3BP, A_7, Del, 44, high expression, NQPQSQARAAHPCHLDLCPHEENLYHQIMSLGSQEVQESFHQAK, (SEQ ID NO: 4693); XM_005626521.2, KIF3A, A_7, Del, 44, high expression, KEGVKRKCPQIRWLKCKQKLMRREKHLKQSLIWKKKKETRLELN, (SEQ ID NO: 4694); XM_005640898.1, PEA15, A_7, In, 44, high expression, NHPGGPGAAQVGLQGGHPQREERGDHDRQRLVQLPGEPQQAGQR, (SEQ ID NO: 4695); XM_014120145.1, PCM1, A_7, Del, 45, driver, high expression, NISQLLLQPQLLMGMKPVQANLLLSLQILQEWIMSCGQKCEDMRC, (SEQ ID NO: 4696); XM_543181.4, CENPJ, A_7, In, 45, essential, RWVHEFISSITEESDFFRNSGRKIQEKSFADTRQSISKIQAYTSS, (SEQ ID NO: 4698); XM_535898.5, NUP153, A_7, Del, 45, essential, NQHSTCLPLERFPLHLGILQSLKQVSLEILLFILEKQRMVGQQLL, (SEQ ID NO: 4699); XM_005641214.2, POLA1, A_7, In, 45, essential, KSTSCTCCPLDEFSRRQKNESWRYCVICHLSGWVKPHCESEGLCT, (SEQ ID NO: 4700); XM_014110420.1, GOLIM4, A_7, Del, 46, high expression, KTTMAKNKKFEMTTAPKAERNPMRRRRRKKKMGLGLQRNHSEERKC, (SEQ ID NO: 4701); XM_544672.5, SECISBP2L, A_7, Del, 46, high expression, KIRHFLEVEGRLNKEIIHRLDSDAEDIVLPQKEDRICRRDKIIISS, (SEQ ID NO: 4702); XM_539598.4, CLSPN, A_7, Del, 46, essential, NCIVRLNALFESLRLIFHIICLRIKPFMISSNVNPDPLARGMPWHY, (SEQ ID NO: 4703); XM_005620337.1, USP24, A_7, Del, 47, high expression, KLPSLKTMNLSVSLSTVGRHMQVTTIPSLRTGGAVEKESGTSLMTQS, (SEQ ID NO: 4705); XM_005626023.2, EIF5B, A_7, Del, 48, essential, high expression, KISQVLMWRVGMKMMIPPSKLRRWPKRRQKRKSVKEKREMKKKQNYGS, (SEQ ID NO: 4707); XM_005629477.2, LUC7L2, A_7, Del, 48, high expression, KYREHRREWEILLVNESNSAMTEYARVTFSTVAPMMSSLELEWILENV, (SEQ ID NO: 4708); XM_005619358.2, ZNF131, A_7, Del, 49, essential, high expression, KFIYVNTVRNSLTTLDILKSIFGNIQVKNLLNVQIVMNDLLEIAPSNVT, (SEQ ID NO: 4710); XM_005641566.1, BTK, A_7, Del, 49, driver, KILPLKDRFQEGVKSPVKWSRFQSLKGSLTPSRLYMMKGLFMSSPQLKN, (SEQ ID NO: 4711); XM_014115688.1, FERMT2, A_7, Del, 50, essential, high expression, KIGLIMLSGGKRRELGFLKHIGPWISMAFRQMLSFSSPPSTNCFAYSFPT, (SEQ ID NO: 4712); XM_534306.5, EIF2A, A_7, In, 50, high expression, NQEPKEETESYRATERTSSSWKTARKKSVGENSKRESPSPGAGRFGIGYL, (SEQ ID NO: 4713); XM_014111409.1, SMARCA1, A_7, Del, 50, high expression, KRLMELNLLHQKRLKKKKNFSHKDLQTGLSEILTSLLKLMRSMEEMTLII, (SEQ ID NO: 4714); XM_003639638.3, POGZ, A_7, In, 50, high expression, IHPKSLEPEGGFLSPAGRRALHGSLSAVSPSPRPLPCSHPLLPAPGARSL, (SEQ ID NO: 4715); XM_535814.5, TTC14, A_7, Del, 50, high expression, KSTIGGGSQVLRGVPLHQQAQTTLVSQLKNLKNTLILDHVISVDMSKDIS, (SEQ ID NO: 4716); XM_535898.5, NUP153, A_7, In, 50, essential, RRNACRQRRVHFRWYRTCPSAICLIVCFGKDRRETAGASHFYFSGVWEES, (SEQ ID NO: 4717); XM_847556.4, BRIP1, A_7, In, 50, driver, TKSMSVLHSTRTNRRCSHNILSLQLSSRCTNKGKYGYQSERTGCHFRRSS, (SEQ ID NO: 4718); XM_014122498.1, NUMA1, A_7, In, 51, driver, essential, high expression, KPKTSLFSGMPSVRAEGNRGVRAGIGQDDYAALIPLHHELQKSQGLGTIRI, (SEQ ID NO: 4719); XM_014111218.1, ZC3H11A, A_7, Del, 51, high expression, KQLWQLFHSFLRTSQSLCPRWKNLETVMCCLQPSLLQTPLHRKSLALLHPK, (SEQ ID NO: 4720); XM_544755.5, MYO9A, A_7, Del, 51, high expression, KTNKIHLILDGMEELGFVRADFQVAPPCLIKTGYLLIQLVANCWREPMEFL, (SEQ ID NO: 4722); XM_014109944.1, WDFY3, A_7, Del, 51, high expression, KWCEMICFITITPMCQKRSKRQMWRLRSQVNSLRHLMISFLKRNPLDTEEP, (SEQ ID NO: 4723); XM_014119514.1, ZMYM4, A_7, Del, 51, high expression, KSFRRGKLLIRGKGLLSFSAPRCASLDIQFHLPAHRLLPPRKLVQAAQKIF, (SEQ ID NO: 4724); XM_005626304.2, RUFY1, A_7, In, 51, high expression, KQPSYVQHETDGRKVTALRKGTARGRGAEPQGAAGAGRPDVRTAAAALAAA, (SEQ ID NO: 4725); XM_537080.5, CDC7, A_7, Del, 51, essential, NMSRILNYQVLKKISRSFMKLYHSLVICLRLRTKLEKALLALFIWPQHSYE, (SEQ ID NO: 4726); XM_014107587.1, NCL, A_7, In, 52, essential, high expression, SRSCHTSQESSCHPWQKGSSYTSQKGGCTSQSSSNTWQEGNHTRQGIGSNPW, (SEQ ID NO: 4727); NM_001012995.2, CAPZA2, A_7, In, 52, high expression, NRWTANHYCMHREPSVPSKKLLEWSLEVRVEVYNHSFNHSSGWHLENSGSLL, (SEQ ID NO: 4728); XM_014109413.1, SLTM, A_7, Del, 52, high expression, KHVLLQEEKIQVLKGIPKISVTPEEMSLHHQEMNLEKRTGEKSEENETRGEL, (SEQ ID NO: 4729); XM_005630416.1, BUB1, A_7, Del, 52, essential, KMQVLLGLGESIRSSPLCHLLFLCLKMETKKIMDYHSLKINPQEPGPLENAL, (SEQ ID NO: 4730); XM_536098.5, MDM4, A_7, Del, 53, driver, essential, high expression, KTPSFLIPATQWNSWIWPTVLKAKKPYQAWENHQITFLNREQIQKTWRIARIS, (SEQ ID NO: 4731); XM_005621312.2, AGL, A_7, Del, 53, high expression, KYKTTLKSYFMCQKTLQILMKSILTWFTNVAYTKIVMELPALGVTISSGLILP, (SEQ ID NO: 4733); XM_531679.5, ZFC3H1, A_7, Del, 53, essential, KILRTQRFFIICANSSSYKIKEIIFFHFWGNLSHPSLNLGLRSIITWICFGIF, (SEQ ID NO: 4734); XM_005625479.2, CDK2, A_7, Del, 53, essential, KSAWTLRRRVYPVLLYERSPCLRSLTTRTLSSYWMSSTQKTNSTWFLNFCTRI, (SEQ ID NO: 4735); XM_014113201.1, MLLT4, A_7, Del, 54, driver, high expression, NQASTSPATRKDQTLTWDLPGQLLAPTMPIGTREKDCLKATRQIYLGALEPLKT, (SEQ ID NO: 4736); XM_005626663.2, PLIN2, A_7, Del, 55, high expression, KLKGLIQFRNQVITLDWAPCLPSSAHVPTNRLSAGLKKPSKKAKRPFLSSIPLLI, (SEQ ID NO: 4738); XM_533729.5, MRPS25, A_7, Del, 55, essential, KSWGRTRKPWKRRSRRKSSFLTQPTSGPESTACGSVSARWRGRSPARACCHYPRS, (SEQ ID NO: 4740); XM_014120446.1, EML4, A_7, Del, 55, driver, KRKKNHKDRGKKKRNLILMIKVHKFEHHLLPSPLHSLSKYTDKLKKARILLPPKA, (SEQ ID NO: 4741); XM_544308.5, TAF7, A_7, Del, 57, high expression, KLFTRQLIYVRCLSPQLMVISILLWRNQLLLLIPKQARKRIRTKRRSLYGTMELLCL, (SEQ ID NO: 4744); XM_844090.4, INO80, A_7, Del, 57, essential, KGEGMKNSPLKNLLVAITTRPKSLPSSLMMHLLLAPRRSTYPSSSLMLVAEKYGLAL, (SEQ ID NO: 4745); XM_005615937.2, CD274, A_7, In, 58, driver, NYTICEWKGRPESSAQQLQPEGSAIEGPALLGEGCASDHRCEIAGCRGLLLLDRLWRC, (SEQ ID NO: 4747); XM_014121199.1, EXT2, A_7, Del, 58, driver, NS SVLSAQPLMGFHLTRRTWWRGLSASTSLLQSLEQCLLRWWSTEQTLSCTKMTFLRN, (SEQ ID NO: 4748); XM_005634561.2, RNF13, A_7, Del, 59, high expression, KPVRSASKKWFLPKATQTPTQTAVKKKMRCPNTPLFLDLWLLSAPSHLGLCRNLAHIRI, (SEQ ID NO: 4749); XM_005632905.2, DNMT1, A_7, Del, 59, essential, KCTRGRKRNRTRIGSLGLEMLSRLMGRRITIRRFASIQKPWKWETVSLLFQMTLQNRCI, (SEQ ID NO: 4750); XM_005626595.2, FAM13B, A_7, Del, 61, high expression, KRNYVKHYGNLKKHFINRMEGMPRKRIVFQCLKSIGSTRKLKPNLGFLKFLSANKILQNPY, (SEQ ID NO: 4752); XM_014119514.1, ZMYM4, A_7, In, 61, high expression, NPSEGANCLSEERVYSAFLLHAVPHWIYSSTCPPTASSHQENLFKLLKRYFKSKGCDQCPV, (SEQ ID NO: 4753); XM_005637209.2, C1R, A_7, In, 61, high expression, NSGEVLWAAGFSTGQPPRSKGIRVSREQDAADLPHRLLQRGEWHHYVPQGLPGLLPGCGPR, (SEQ ID NO: 4754); XM_003639701.3, NIFK, A_7, Del, 61, essential, KMLQTLVLRSLQIARSCRKRRRRRRRVHVLLPLLRRPWIARAPHQFVHQHFWRNENLKQLK, (SEQ ID NO: 4755); XM_005634135.2, TMCO3, A_7, In, 62, high expression, KLLLDVASTASFDSRCCRTRRGSKTCHQVTPRQRSGHRSEEAVGPGQLQKALWASSPKGCGS, (SEQ ID NO: 4756); XM_005615620.2, RSPO3, A_7, Del, 62, driver, KEGRGKGKNPIRKRVRTQYLTTKVWNPAEKPRNHEKTKTNS SRRSERSKINNRNRLWISFGD, (SEQ ID NO: 4757); XM_005634448.1, TOPBP1, A_7, Del, 64, essential, KSSKDKNQALLRGHLVGGRLSYMLTSPEKQDSNAFFSQEEQRYYLVILYLYLKKLHISFLTSIN, (SEQ ID NO: 4759); XM_005615469.2, PHACTR2, A_7, Del, 66, high expression, KLPRLLVSRPPSLHPSQQAETRPERLLVPLIQKKQLGPNHQLHHLLPPPHLVPELRRRHSLAKQGQ, (SEQ ID NO: 4760); XM_014113193.1, ATM, A_7, Del, 67, driver, high expression, KSLLKRNMKLSWISAKNFSQFSVTSAWKSSWTQLFGLRSDWLILAVWPLPRLLVTYLDLVIDMCRI S, (SEQ ID NO: 4761); XM_536310.6, ZFYVE16, A_7, Del, 68, high expression, KIQFWLNKGYLTVNLRLQMNYQSLILTISLFMLEGPDQSNCLTFHQDQEVQRNQINQMFQICLKVSP A, (SEQ ID NO: 4763); XM_532445.5, WASL, A_7, Del, 69, high expression, NSEKQLQTCWAGDKGNLRRGETPQMVLIYPWQQLTSKIQKSQPIDFMVHKSITSPIPKKRKKEKLK RRD, (SEQ ID NO: 4765); XM_014116784.1, SUPT6H, A_7, Del, 70, essential, high expression, KCQMTRMTTRRNMAKRNMKKRLLQRKSSRMGKGKKGRKLWRPPWLLQRRRRKMMKNQTSMT LLWMMTDSP, (SEQ ID NO: 4766); XM_540739.5, FNBP4, A_7, Del, 71, essential, high expression, KQSYEPWRKEMVVCQGLVHVLTSASQHLRMECVDLCLKGGNGRCLFELQVQNLRAGVLAKLDG RLQKMEKL, (SEQ ID NO: 4768); XM_534457.5, ATP9A, A_7, Del, 71, high expression, KTGLVSCGRISWTGRQIGSCGSPWPARRGSPLLPISFRFDHMSMQKSQISTFTTLWELLPERTATLRS ARV, (SEQ ID NO: 4769); XM_014121880.1, MAP4, A_7, Del, 71, high expression, KQRHLLRLESLNLTQSLKQLVPLRVHRNHLLGKFRFRIRRWTSLRSPPNVGPRLTSSTNLVEETSRL KVRS, (SEQ ID NO: 4770); XM_537657.5, CWC25, A_7, Del, 71, essential, KRRRRKRRKRKRSTRNISTEAQVVMVAVARMSAVGGDLKRSWQILPLCCPKPLDMAYRSGTPVIA REYRVL, (SEQ ID NO: 4771); XM_005636081.1, SFSWAP, A_7, Del, 72, essential, KRRRGAHGHGPSPRPGPSRRPQASKPHVGTRPTQPASLPWRVGAPVRSAPGEFLRKKTARSPQQLS PLCRAK, (SEQ ID NO: 4772); XM_536224.5, WHSC1, A_7, Del, 72, driver, KESTQRGHRSLLKTLTAKMHPGRDSGPTSTVFGRERRSMTRQPEQAPARPSRQPPHSRARQQRNIC QMHANH, (SEQ ID NO: 4774); XM_534457.5, ATP9A, A_7, In, 73, high expression, KRVLFPADGSAGRGDRLEAAAPRGLHAEAPHCCRSPSDSIICLCRRAKYRHSQLCGNFYQRGQRPS DQREFEH, (SEQ ID NO: 4775); XM_005640898.1, PEA15, A_7, Del, 73, high expression, KSPWRTWSSSSRPARRTSPARRARRSRPAAPGSASWRATTSWTKITSRTSSTSLRSPAAPTSSPWWW TTEPAS, (SEQ ID NO: 4776); XM_003639982.3, TNKS2, A_7, Del, 74, high expression, NCVLFRVSTAETLKGVSLRHSILQLAIIGYLWWSICCNMELTCMLKIKEALYLCTMLVLTDIMKLQN FLLSMEQ, (SEQ ID NO: 4777); XM_537556.5, DYNC1H1, A_7, Del, 75, essential, high expression, KRSQNLKWGSFTCSKILKFQRSACQFIPLLQMLQNNVMNVEKSQKLQTLEIRLKTQLFSISYNQELT VGYEKFKK, (SEQ ID NO: 4778); XM_544733.5, DENND4A, A_7, Del, 75, high expression, KCSRKRWIHLMKCATAFSCSSVGSTISPCLQFECFLKCRKLVLIPMPLLMVIIIRLFWKAPGPQEVVV AIFFGQK, (SEQ ID NO: 4779); XM_005637236.2, CHD4, A_7, Del, 76, driver, essential, high expression, KRSWGTALGRGRSLWRRRKRWLFAQTVRAATIPLARRRRRSLDLRKKRRANPSGRKRKKRMTTT MIQRSLNHLLSS, (SEQ ID NO: 4780); XM_860109.5, RBM39, A_7, Del, 79, essential, high expression, KARAEVVVMNEKEAKVRNGNEVEIEKGKRAKVAKGSEVGAKKGDGAAQEVEIADSEAATEVLTD DAPEAKVHSEKTRAP, (SEQ ID NO: 4781); XM_014118080.1, DST, A_7, Del, 80, high expression, KRDVIPSVPLCPLLPRTQLEMRYLISMLLESRRICLLRRDCYCGHSRQQRVTLEYGVKISLPAGEME NYLMPSFINTGRT, (SEQ ID NO: 4782); XM_005631747.1, SART1, A_7, In, 80, essential, TPWGERGGRDDGGGQHRRRHRAAAAAPGAQKTQAPERRWREQRWRTSEAESGAWGRARGRAA RDRSRDTQRRARAGARPG, (SEQ ID NO: 4783); XM_005637237.2, CHD4, A_7, Del, 83, driver, essential, high expression, KRSVCSYAGSWGTALGRGRSLWRRRKRWLFAQTVRAATIPLARRRRRSLDLRKKRRANPSGRKR KKRMTTTMIQRSLNHLLSS, (SEQ ID NO: 4785); XM_005615711.2, HDDC2, A_7, Del, 83, high expression, KSLPTWPCVPLRPNHPEAWHPCPTLESCTKLTTPRPGLYYHCGPSPSISSGTGESPGRAGYTEASRGR RVYRITCTEWQLWLW, (SEQ ID NO: 4787); XM_863061.4, PNISR, A_7, Del, 83, essential, KSIKRKRNKEGVDQEVLVVVVPVAIAELVVLVVLSLALHIVLAQVVVALLLDLLLLKGKKDIVGVD LQQLKLDGVEVEVTHAE, (SEQ ID NO: 4788); XM_014116966.1, RALGDS, A_7, Del, 83, driver, KSSGSPPPSHPRRPPASAQPPAAPPPPQPPPRPCPPHAPTSAPSQGSAATAPHCRSTTSRWATAASSAS AWTWTTATCTRASW, (SEQ ID NO: 4789); XM_003639390.3, TLN1, A_7, Del, 84, essential, high expression, KARIILGWRETRSLLCWRTQFPPKSRQSSSSSITGWGRWSTALWPCLLSCALEPPVLKISRWAACHQ PSSRLPVARCTEDTCPL, (SEQ ID NO: 4791); XM_005615469.2, PHACTR2, A_7, Del, 90, high expression, KHLCLQKGLLLGQAPRLTKCLLLKKLPRLLVSRPPSLHPSQQAETRPERLLVPLIQKKQLGPNHQLH HLLPPPHLVPELRRRHSLAKQGQ, (SEQ ID NO: 4797); XM_005622426.1, RGL1, A_7, In, 91, high expression, KALRVFLVVFLYPFHGHHFLRDVVLNQPPLLPSVLQQQPQNPQALRLCDIHYLDSAASRLQPTERR HLHNPHQCRGQQRQHVQEHRADEPR, (SEQ ID NO: 4798); XM_005618005.1, EXOSC10, A_7, Del, 96, essential, high expression, KQNLKLFGCFMQKTSSGLSLNSEKRSTTPTLRFFPRSSSSPMLRNPSLRHSRKKGESARRIVRRTWM SPLPWLISSINREPSRWSRTCLHILTSMN, (SEQ ID NO: 4802); XM_005636081.1, SFSWAP, A_7, In, 96, essential, KEEEALTVTDRVQGRVPVGVPKQASHTSAHGPLSQHLSRGESGLQSGALQGSFSGKRRPDLLSNCL LCAEQNNSGPHGQSEGDARCFQEHADQCLL, (SEQ ID NO: 4803); XM_536972.4, RSL1D1, A_7, Del, 97, essential, high expression, KRVLVPTDPVEKRERPVQVWRPRRLQRPRLQVTAQRRSQESKKRQRKKETLHWGKKTQDRRPKS QEPGSLLLLINLQKKLPTPPNSGPKSAKYPSQP, (SEQ ID NO: 4804); XM_005633841.2, ALG11, U_7, Del, 8, essential, high expression, HHYSSLG, (SEQ ID NO: 4805); NM_001287163.1, HIF1A, U_7, In, 8, driver, high expression, SQNEVYPN, (SEQ ID NO: 4806); XM_005625788.2, ARFGAP3, U_7, Del, 8, high expression, LPLMFLLR, (SEQ ID NO: 4807); XM_005639633.1, ATP13A3, U_7, In, 8, high expression, LGQAATLV, (SEQ ID NO: 4808); XM_014111351.1, DMD, U_7, In, 8, high expression, FWSSCKRP, (SEQ ID NO: 4809); XM_014109438.1, MYO9A, U_7, Del, 8, high expression, FIFPNKTH, (SEQ ID NO: 4810); XM_539577.5, ZMPSTE24, U_7, Del, 8, high expression, LYMPGCSH, (SEQ ID NO: 4811); XM_005623314.2, ARHGAPS, U_7, Del, 8, high expression, LKMRLSRN, (SEQ ID NO: 4812); XM_005618393.2, BTBD1, U_7, In, 8, high expression, FLFQLPWQ, (SEQ ID NO: 4813); XM_005631715.2, RCN1, U_7, Del, 8, high expression, FCFLFFFF, (SEQ ID NO: 4814); XM_005619936.2, RNASEK, U_7, Del, 8, high expression, FSTSIPLC, (SEQ ID NO: 4815); XM_003640168.3, ABT1, U_7, Del, 8, essential, FSSPRTGS, (SEQ ID NO: 4816); XM_014107997.1, KNTC1, U_7, Del, 8, essential, FTLQIFNF, (SEQ ID NO: 4817); XM_014114799.1, ECT2L, U_7, Del, 8, driver, FYDAPNHN, (SEQ ID NO: 4818); XM_538482.4, MSH2, U_7, Del, 8, driver, LQKIFIRT, (SEQ ID NO: 4819); XM_005625977.2, RANBP2, U_7, In, 9, driver, essential, high expression, LQSCKIVSF, (SEQ ID NO: 4820); XM_005617468.2, BDP1, U_7, In, 9, essential, high expression, CSFASESSC, (SEQ ID NO: 4821); XM_005630347.2, CEBPZ, U_7, In, 9, essential, high expression, SDLYQEKRY, (SEQ ID NO: 4822); XM_005639064.2, USO1, U_7, Del, 9, essential, high expression, LKKAHIFNV, (SEQ ID NO: 4823); NM_001002952.1, EPB41L5, U_7, In, 9, high expression, LAQDNQIGF, (SEQ ID NO: 4824); XM_005640282.2, LRP2, U_7, In, 9, high expression, FRYIKRHDL, (SEQ ID NO: 4825); XM_005640225.2, MARCH7, U_7, Del, 9, high expression, FLEDLARIP, (SEQ ID NO: 4826); XM_005619828.2, ACAT1, U_7, In, 9, high expression, FLSALRWYF, (SEQ ID NO: 4827); XM_005639074.2, ART3, U_7, Del, 9, high expression, LIKKVKKLF, (SEQ ID NO: 4828); XM_547836.5, DAAM1, U_7, Del, 9, high expression, LKCSEMKMS, (SEQ ID NO: 4829); XM_005627538.2, FILIP1, U_7, Del, 9, high expression, FLVPLSFLP, (SEQ ID NO: 4830); NM_001289066.1, MME, U_7, Del, 9, high expression, LLARMIRTL, (SEQ ID NO: 4831); XM_014120908.1, SERPING1, U_7, In, 9, high expression, FLWLCSRIL, (SEQ ID NO: 4832); XM_014111948.1, TSPAN6, U_7, Del, 9, high expression, FWLNWSLPS, (SEQ ID NO: 4833); XM_014114990.1, DIEXF, U_7, In, 9, essential, CEQDFASVS, (SEQ ID NO: 4834); XM_536500.4, NUP155, U_7, In, 9, essential, FGWKRWLFI, (SEQ ID NO: 4835); XM_005621512.2, SMG1, U_7, Del, 9, essential, LMMIITGRC, (SEQ ID NO: 4836); XM_014119539.1, ABCE1, U_7, Del, 10, essential, high expression, FWMATFQQKT, (SEQ ID NO: 4837); XM_005638404.2, TRPM7, U_7, In, 10, essential, high expression, FNRRRSKETT, (SEQ ID NO: 4838); XM_531814.5, MSH6, U_7, Del, 10, driver, high expression, LEMTLYPMTF, (SEQ ID NO: 4839); XM_005623082.2, ANKRD12, U_7, Del, 10, high expression, LKVMRLKICF, (SEQ ID NO: 4840); XM_003431626.3, FNIP1, U_7, Del, 10, high expression, FHIFLSLKAT, (SEQ ID NO: 4841); XM_014119426.1, FNIP2, U_7, Del, 10, high expression, FLIFPCLNLT, (SEQ ID NO: 4842); XM_005629907.2, LEPROTL1, U_7, Del, 10, high expression, FTSFHLFHTA, (SEQ ID NO: 4843); XM_005626107.2, PSME4, U_7, Del, 10, high expression, LLRISTMMQL, (SEQ ID NO: 4844); XM_005623486.2, SYNE2, U_7, Del, 10, high expression, FSRNLLLTCC, (SEQ ID NO: 4845); XM_005638476.2, VPS13C, U_7, Del, 10, high expression, FIIDKIMALY, (SEQ ID NO: 4846); XM_005641570.2, ARMCX1, U_7, In, 10, high expression, FLIQRIWSVC, (SEQ ID NO: 4847); XM_005623379.1, SOS2, U_7, In, 10, high expression, WNIFNKYSEN, (SEQ ID NO: 4848); XM_014111151.1, MFN2, U_7, Del, 10, essential, LAGRATGRAL, (SEQ ID NO: 4849); XM_005635483.2, USPL1, U_7, In, 10, essential, FHFSIKFFSK, (SEQ ID NO: 4850); XM_005625977.2, RANBP2, U_7, Del, 11, driver, essential, high expression, FAIVQNCFVLM, (SEQ ID NO: 4851); XM_014114127.1, CCT6A, U_7, Del, 11, essential, high expression, FTRVQKRERNS, (SEQ ID NO: 4852); XM_849291.3, PRPF8, U_7, Del, 11, essential, high expression, FSGSQQMRLGT, (SEQ ID NO: 4853); XM_014111447.1, VPS13D, U_7, Del, 11, essential, high expression, LELVPLPTTMQ, (SEQ ID NO: 4854); XM_014115842.1, BAZ1A, U_7, In, 11, high expression, FLPDCHLPGDS, (SEQ ID NO: 4855); XM_532457.5, CYP51A1, U_7, In, 11, high expression, FGAEENAKKWP, (SEQ ID NO: 4856); XM_533330.4, SLC35F5, U_7, In, 11, high expression, FIFYCNSLMPL, (SEQ ID NO: 4857); XM_846785.4, BTBD10, U_7, In, 11, high expression, FLFLCVYLGIY, (SEQ ID NO: 4858); NM_001003359.1, CAMP, U_7, Del, 11, high expression, LRIFSPGRRSP, (SEQ ID NO: 4859); XM_862279.4, G3BP1, U_7, In, 11, high expression, SKLWECGGATH, (SEQ ID NO: 4860); XM_005635381.2, PARP4, U_7, Del, 11, high expression, LQQKIMKHQNF, (SEQ ID NO: 4861); XM_005636714.2, ASUN, U_7, Del, 11, essential, FCMSLAVLDPF, (SEQ ID NO: 4862); XM_003639984.3, BTAF1, U_7, In, 11, essential, FGEGSSMAGSN, (SEQ ID NO: 4863); XM_536500.4, NUP155, U_7, Del, 11, essential, FWLEKMAVYMK, (SEQ ID NO: 4864); XM_005618818.2, SPRTN, U_7, In, 11, essential, YQERASTKWWK, (SEQ ID NO: 4865); XM_014120337.1, BIRC6, U_7, Del, 12, essential, high expression, FYIMLIEYLSFH, (SEQ ID NO: 4866); XM_005634821.2, PANK2, U_7, In, 12, high expression, WSLLSSYWLYHF, (SEQ ID NO: 4867); XM_005635476.2, PDS5B, U_7, Del, 12, high expression, FSSILTKMFAYW, (SEQ ID NO: 4868); XM_014109945.1, WDFY3, U_7, In, 12, high expression, LLTRIPVQFQQL, (SEQ ID NO: 4869); XM_014113785.1, ZCCHC2, U_7, In, 12, high expression, SASDSLCKRTGA, (SEQ ID NO: 4870); XM_536110.5, CFH, U_7, Del, 12, high expression, FLNMNLHMSWLQ, (SEQ ID NO: 4871); XM_847415.3, CLPX, U_7, Del, 12, high expression, LLCYLKQTQRKA, (SEQ ID NO: 4872); XM_014110499.1, GMDS, U_7, In, 12, high expression, LRSPRHSPQAMV, (SEQ ID NO: 4873); XM_005637041.2, TM7SF3, U_7, Del, 12, high expression, LDTDSGKQNYFS, (SEQ ID NO: 4874); XM_014117483.1, LIPT1, U_7, Del, 12, essential, LQPKKSMIECRI, (SEQ ID NO: 4875); XM_014114127.1, CCT6A, U_7, In, 13, essential, high expression, LQECRRERETRKS, (SEQ ID NO: 4876); XM_014118082.1, DST, U_7, Del, 13, high expression, LVVNFISQKTWQI, (SEQ ID NO: 4877); XM_005631134.2, HIPK3, U_7, Del, 13, high expression, FAEKQRFLILVGD, (SEQ ID NO: 4878); XM_014109991.1, SPARCL1, U_7, In, 13, high expression, FPIHFGNCSCNPG, (SEQ ID NO: 4879); XM_005635807.1, TRIP12, U_7, Del, 13, high expression, FLHHFLEKSPLEE, (SEQ ID NO: 4880); XM_846785.4, BTBD10, U_7, Del, 13, high expression, FLVSVCLPWHILK, (SEQ ID NO: 4881); XM_536110.5, CFH, U_7, Del, 13, high expression, FQQSMEIRQHVLM, (SEQ ID NO: 4882); XM_537465.5, DHRS7, U_7, Del, 13, high expression, LIASGLNLLSTQV, (SEQ ID NO: 4883); XM_848657.4, TRIM38, U_7, Del, 13, high expression, FWTVILELYPFTT, (SEQ ID NO: 4884); XM_014113993.1, CENPN, U_7, Del, 13, essential, LRCLLSSSRSNRA, (SEQ ID NO: 4885); XM_005631620.2, MTA2, U_7, Del, 13, essential, FTHWCLTLCRRHF, (SEQ ID NO: 4886); XM_005630848.2, SLC16A1, U_7, Del, 13, essential, LQLLLLQMECVIC, (SEQ ID NO: 4887); XM_535270.5, TNPO1, U_7, Del, 14, essential, high expression, FVMLLHRGLTQKMI, (SEQ ID NO: 4888); XM_014109908.1, ANK2, U_7, In, 14, high expression, CLQRKPTSSLCQGT, (SEQ ID NO: 4889); XM_536312.4, CMYA5, U_7, Del, 14, high expression, LTPSRRTVVKMRKG, (SEQ ID NO: 4890); XM_005627518.2, PHF3, U_7, Del, 14, high expression, FGTPMLNIRTNTEV, (SEQ ID NO: 4891); XM_005637011.2, TMTC1, U_7, In, 14, high expression, FHKRKPTKRAESSG, (SEQ ID NO: 4892); XM_005638321.2, UBR1, U_7, Del, 14, high expression, LWRWNTKNSLLWNL, (SEQ ID NO: 4893); XM_014109945.1, WDFY3, U_7, Del, 14, high expression, FTYQNSCSIPTTLI, (SEQ ID NO: 4894); XM_005618393.2, BTBD1, U_7, Del, 14, high expression, FSFSAPLAIIMALQ, (SEQ ID NO: 4895); XM_856309.4, KDELR2, U_7, In, 14, high expression, FITMYKDFFTQRNL, (SEQ ID NO: 4896); XM_856309.4, KDELR2, U_7, Del, 14, high expression, FYYYVQRFFYTKKL, (SEQ ID NO: 4897); XM_014119142.1, LSM8, U_7, In, 14, high expression, FPRLKKPRGNTERV, (SEQ ID NO: 4898); XM_005631715.2, RCN1, U_7, In, 14, high expression, FVFCFFFSKLQEDR, (SEQ ID NO: 4899); XM_014107997.1, KNTC1, U_7, In, 14, essential, LHYKSSTFKNSTSD, (SEQ ID NO: 4900); XM_005624950.2, MNT, U_7, In, 14, essential, FNLQILFCKYFGKH, (SEQ ID NO: 4901); XM_005640646.2, RQCD1, U_7, In, 14, essential, FVPLFAHCQQNTTL, (SEQ ID NO: 4902); XM_005625561.2, LRIG3, U_7, Del, 14, driver, FPAMYSLICKHLAS, (SEQ ID NO: 4903); XM_005616941.2, CUL2, U_7, In, 15, essential, high expression, FGKSRTAFAQESSGV, (SEQ ID NO: 4904); XM_014111447.1, VPS13D, U_7, In, 15, essential, high expression, WNWFHCRQPCNEGAA, (SEQ ID NO: 4905); XM_014115839.1, BAZ1A, U_7, Del, 15, high expression, LNEPFCATVLCGVVL, (SEQ ID NO: 4906); XM_005630949.2, BCAP29, U_7, In, 15, high expression, LASVETSGYPYYSIG, (SEQ ID NO: 4907); XM_014118119.1, PHIP, U_7, In, 15, high expression, WIRSARENIRIGVPY, (SEQ ID NO: 4908); XM_535696.4, ENPEP, U_7, In, 15, high expression, SQNKPRSYWVLSCKL, (SEQ ID NO: 4909); XM_014106285.1, MYCBP2, U_7, Del, 15, high expression, FPRNLKLILMTFSKI, (SEQ ID NO: 4910); XM_843573.4, DARS, U_7, In, 16, essential, high expression, FGIAQCSSDVNVPPGS, (SEQ ID NO: 4911); XM_005629907.2, LEPROTL1, U_7, In, 16, high expression, LHPFTYSILHSEKISG, (SEQ ID NO: 4912); XM_014121460.1, SLC35F5, U_7, Del, 16, high expression, LQMLKVTLLLAQQIRL, (SEQ ID NO: 4913); XM_005641570.2, ARMCX1, U_7, Del, 16, high expression, FSYSKNLECVLRKSRH, (SEQ ID NO: 4914); NM_001003198.1, PLA2G7, U_7, Del, 16, high expression, LVLVNILEHPGLWAKY, (SEQ ID NO: 4915); XM_532688.5, GATB, U_7, Del, 16, essential, LMHLCLELSRFSTGGA, (SEQ ID NO: 4916); XM_005625825.2, XRCC6, U_7, Del, 17, essential, high expression, FWLMVLGPCLNLRVKLN, (SEQ ID NO: 4917); XM_533330.4, SLC35F5, U_7, Del, 17, high expression, FHFLLQLSYAIIIIGIL, (SEQ ID NO: 4919); XM_014119604.1, TMPO, U_7, In, 17, high expression, FVFGLSSYGNQPRKSVQ, (SEQ ID NO: 4920); XM_005635807.1, TRIP12, U_7, In, 17, high expression, FFTTSWRRAHWKSRTSG, (SEQ ID NO: 4921); XM_005638321.2, UBR1, U_7, In, 17, high expression, YGDGIQKILCYGICEVL, (SEQ ID NO: 4922); XM_535047.6, PSTK, U_7, Del, 17, essential, FIMRKITLSGNIFQSHT, (SEQ ID NO: 4923); XM_005640494.1, SF3B1, U_7, Del, 18, driver, essential, high expression, LNTSGNTEWLWTEEITDS, (SEQ ID NO: 4924); XM_535470.5, COPS2, U_7, Del, 18, essential, high expression, LKPSRIMMNREVQDEPLA, (SEQ ID NO: 4925); XM_005630347.2, CEBPZ, U_7, Del, 18, essential, high expression, FGLVSRKKILNQKCLVPF, (SEQ ID NO: 4926); XM_005618617.2, UGDH, U_7, Del, 18, high expression, FLPILTMPLKKLILYLFL, (SEQ ID NO: 4927); XM_014112771.1, DNA2, U_7, Del, 18, essential, FLRDLCWWGIINSFLPWC, (SEQ ID NO: 4929); XM_014119539.1, ABCE1, U_7, In, 19, essential, high expression, FGWLRSNRKLEIQRCLTCF, (SEQ ID NO: 4930); XM_544475.5, NUDC, U_7, In, 19, essential, high expression, CWRRRGDGREAHHTDLQPP, (SEQ ID NO: 4931); XM_005631134.2, HIPK3, U_7, In, 19, high expression, LQRNRDFSFWLEIKDTRRT, (SEQ ID NO: 4932); XM_014109990.1, SPARCL1, U_7, In, 19, high expression, FPIHFGNCSCNPDKCKVPV, (SEQ ID NO: 4933); XM_014109991.1, SPARCL1, U_7, Del, 19, high expression, FSYTFWELQLQSRLKNLLY, (SEQ ID NO: 4934); XM_539577.5, ZMPSTE24, U_7, In, 19, high expression, YICLAVHISCFSGACHDLC, (SEQ ID NO: 4935); XM_005619936.2, RNASEK, U_7, In, 19, high expression, FQRPFRCADRGRSFHRERF, (SEQ ID NO: 4937); XM_014118274.1, MED23, U_7, Del, 19, essential, LQALIQFRELGVKTYFRPS, (SEQ ID NO: 4938); XM_005624950.2, MNT, U_7, In, 19, essential, LHQNFFNLQILFCKYFGKH, (SEQ ID NO: 4939); XM_005640646.2, RQCD1, U_7, Del, 19, essential, FCTPFCTLSAKHDPLNIFD, (SEQ ID NO: 4940); XM_005641414.2, AMER1, U_7, Del, 19, driver, LAVSAVTGKARSLGLKKVS, (SEQ ID NO: 4941); XM_535048.6, ACADSB, U_7, In, 20, high expression, FLYPRDRGISQSRCICRVGL, (SEQ ID NO: 4943); XM_014116675.1, AKAP1, U_7, Del, 20, high expression, FLVKSMLAVRTDRWRPALWS, (SEQ ID NO: 4944); XM_005624276.2, GNA13, U_7, In, 20, high expression, FAVSPYHKSTVGRQWHTECL, (SEQ ID NO: 4945); XM_845208.4, HECTD1, U_7, In, 21, essential, high expression, FGSASKTWCNQQSINFGRSFL, (SEQ ID NO: 4947); XM_005640284.2, LRP2, U_7, Del, 21, high expression, FQLTLVVSTSKKSFLLMIQRI, (SEQ ID NO: 4948); XM_005640538.2, CFLAR, U_7, Del, 21, high expression, FCAGTLLQMLLHLLSGTFWIF, (SEQ ID NO: 4949); XM_538482.4, MSH2, U_7, In, 21, driver, YKRYLSGLEPVIERQKGRTGE, (SEQ ID NO: 4951); XM_005638158.1, KIAA1429, U_7, In, 22, essential, high expression, FWTHTTFRAAASTHHANNSGY, (SEQ ID NO: 4952); XM_843573.4, DARS, U_7, Del, 22, essential, high expression, FWDCTMFVRRQCSPGILNDSLL, (SEQ ID NO: 4953); XM_005639633.1, ATP13A3, U_7, In, 22, high expression, CCVCDYFVCFHITHHVASSCLY, (SEQ ID NO: 4954); XM_014115201.1, KIFAP3, U_7, Del, 22, high expression, FAFLAFLSFMDLSLTIKLELYV, (SEQ ID NO: 4955); XM_005639457.2, CD47, U_7, In, 22, high expression, FICWWTSAHYSCHCRSHSFRPR, (SEQ ID NO: 4956); XM_005640538.2, CFLAR, U_7, In, 22, high expression, FVPGRCCRCCSTYCQGPSGYFK, (SEQ ID NO: 4957); XM_005624315.2, BRCA1, U_7, Del, 22, driver, essential, LLTIQVLVSLKSASTLAFEEKK, (SEQ ID NO: 4959); XM_014116993.1, NUP214, U_7, Del, 22, driver, essential, LMFAHFQMRLNSKNAHLPIISF, (SEQ ID NO: 4960); XM_005626903.2, IKBKAP, U_7, Del, 22, essential, LSMTLRLLQISHHLQYMMNFYC, (SEQ ID NO: 4961); XM_014106286.1, MYCBP2, U_7, Del, 23, high expression, FPRKQQCKNKICRSCEEGQAYTR, (SEQ ID NO: 4962); XM_005638361.2, CTDSPL2, U_7, Del, 23, essential, FHQQIKMEHQDPIPQDRLWKLKK, (SEQ ID NO: 4963); XM_005617468.2, BDP1, U_7, Del, 24, essential, high expression, LLICFRKFLLKKRKENKNLLKIRA, (SEQ ID NO: 4964); XM_846518.4, C4BPB, U_7, Del, 25, high expression, FAMPLWSGTPPFPHVSWATVLSLCW, (SEQ ID NO: 4965); XM_536312.4, CMYA5, U_7, Del, 25, high expression, FTRTYMNKQLERKTRKRRQLLKVTV, (SEQ ID NO: 4966); XM_005641779.2, STAG2, U_7, Del, 26, driver, essential, high expression, FSFWKVSCMSMQHTLWIACGTVLPSY, (SEQ ID NO: 4967); XM_014112649.1, RBPJ, U_7, In, 26, essential, high expression, LPSSLCVSYGQWMEEKKRTNGTGWLF, (SEQ ID NO: 4968); NM_001171748.1, RBM12, U_7, Del, 26, essential, high expression, LKSWILWKIVFILLMDPMGKQLVKAL, (SEQ ID NO: 4969); XM_014120154.1, FAT1, U_7, Del, 26, driver, high expression, FQYILKQVLSQRRQGNWTESSKMNIY, (SEQ ID NO: 4970); XM_847331.4, CEPT1, U_7, Del, 26, high expression, FVVLLGHLCSIVHTGRRMFLEHCDLE, (SEQ ID NO: 4971); XM_014109438.1, MYO9A, U_7, In, 26, high expression, LYSPTRPTENKFPTRYKYPEKQAIRR, (SEQ ID NO: 4972); XM_005628406.2, SMO, U_7, Del, 26, driver, LWGALAGWPSLWTALAGRLSAVQMAP, (SEQ ID NO: 4973); NM_001031630.1, TERT, U_7, Del, 26, driver, FMSRKPRFKRTDSSSTGRASGASYRA, (SEQ ID NO: 4974); XM_014109908.1, ANK2, U_7, Del, 27, high expression, LPSKKTDFLSLSRYVIRLRNLAGDYHL, (SEQ ID NO: 4975); XM_846518.4, C4BPB, U_7, In, 27, high expression, LQCLSGVERPRSHMSVGPLSCPCAGEW, (SEQ ID NO: 4976); XM_847331.4, CEPT1, U_7, In, 27, high expression, LLFCWDIYVLLCTLADVCFWNIAIWNN, (SEQ ID NO: 4977); XM_003639984.3, BTAF1, U_7, Del, 27, essential, FWRRFFHGWEQLMTISNRRVQLKHLHV, (SEQ ID NO: 4978); XM_005620968.2, WBSCR22, U_7, Del, 27, essential, FLRFTLCWFMEPGPSCSYTRRTPSSWS, (SEQ ID NO: 4979); XM_014116024.1, PCNX, U_7, In, 28, high expression, FYSTKSLSGKCNIHHFICSTCEILGERL, (SEQ ID NO: 4980); XM_014111846.1, PRDX4, U_7, Del, 28, high expression, FSSTPLISPLCVQLKSSPLVIELKNSNR, (SEQ ID NO: 4981); XM_014118218.1, REV3L, U_7, Del, 28, essential, FQIQVLRVVIHLKIACLLNIIIILILIQ, (SEQ ID NO: 4982); XM_005639026.2, MCM3AP, U_7, Del, 28, essential, LITRLQPWPGRRGKACIKTWLSFGTRRK, (SEQ ID NO: 4983); XM_535002.5, NT5C2, U_7, Del, 29, driver, high expression, LLIVLDIPAVKQDLKMGTSSCLTEVCSRM, (SEQ ID NO: 4984); XM_014109990.1, SPARCL1, U_7, Del, 29, high expression, FSYTFWELQLQSRQMQGSCLIIPNQLLIL, (SEQ ID NO: 4985); XM_014119604.1, TMPO, U_7, Del, 29, high expression, FCFWSIKLWKPTKEIRSVVFFLMTLVNPS, (SEQ ID NO: 4986); XM_005630079.2, EIF2AK2, U_7, Del, 29, high expression, LLKNLIYSVRSTERYLSIMNCLRKDLHIT, (SEQ ID NO: 4987); XM_005634821.2, PANK2, U_7, Del, 30, high expression, LVSAVFLLAVPLLKKLLKWHLVEIAPKWIN, (SEQ ID NO: 4989); XM_014109072.1, OBFC1, U_7, In, 30, essential, LQWASNKAGRDLGNCNWKERKRCFLQLWSG, (SEQ ID NO: 4990); XM_014118473.1, MRPL14, U_7, Del, 31, essential, high expression, LLGSGAPSLMQAEHSASAVSAPLGASVQFRR, (SEQ ID NO: 4991); XM_014118119.1, PHIP, U_7, Del, 31, high expression, LDQVSQRKYQNWSSILTKLTVSSFPTLATDL, (SEQ ID NO: 4992); XM_005637041.2, TM7SF3, U_7, In, 31, high expression, WTQILENRIIFHRFYLHGILLLHTDYKTDTY, (SEQ ID NO: 4993); XM_014107328.1, PARP4, U_7, Del, 32, high expression, FCLFLKEFLLLVHPEGLTFITILILLFIFKIL, (SEQ ID NO: 4994); XM_014119949.1, TBXAS1, U_7, In, 32, high expression, LPGFLGKPNGAQKAIWTSVWVLSWSPDVYCYL, (SEQ ID NO: 4995); NM_001031630.1, TERT, U_7, In, 33, driver, LCHGNHVSKEPTLLLPEERLEPVTEHRNQTTLQ, (SEQ ID NO: 4998); XM_534667.5, RNF34, U_7, In, 34, high expression, FKPYSPFCYCVFISGRLYGWRPDLGIWSSGTGTK, (SEQ ID NO: 5000); XM_014118865.1, UTRN, U_7, Del, 34, high expression, FRVERQKVTNYITQWWNIVYLQHLGKMYETSQRC, (SEQ ID NO: 5001); NM_001012395.1, UTRN, U_7, Del, 34, high expression, FRVERQKVTNYITQWWNIVYLQHLGKMYETSQRY, (SEQ ID NO: 5002); XM_005624915.2, GEMIN4, U_7, In, 34, essential, LYPGILSRSAFIEIGHAPPRGVRAAVCLGLGNPHLL, (SEQ ID NO: 5004); XM_005628406.2, SMO, U_7, In, 34, driver, CGEHWLVGPVYGRRSPGDCLPCRWHHETWGAHLQ, (SEQ ID NO: 5005); XM_014117404.1, RANBP2, U_7, Del, 35, driver, essential, high expression, FVESAVILMRTMEMGKIFSQNFKKFRTLRNPRMRK, (SEQ ID NO: 5006); XM_005621786.2, CEPT1, U_7, Del, 35, high expression, FVVLLGHLCSIVHTGRRMFLEHCDLEYSMLQSPRS, (SEQ ID NO: 5007); XM_534336.5, CSRP2BP, U_7, Del, 35, essential, FGLALTCLSAYSIQTSVLLSFIKKSSLPLASWYLM, (SEQ ID NO: 5009); XM_014118865.1, UTRN, U_7, In, 36, high expression, FGSNGKRSQITLPNGGILYTYNIWGRCTRLHKGAEE, (SEQ ID NO: 5011); NM_001012395.1, UTRN, U_7, In, 36, high expression, FGSNGKRSQITLPNGGILYTYNTWGRCTRLHKGTEE, (SEQ ID NO: 5012); XM_014119949.1, TBXAS1, U_7, Del, 36, high expression, FARVSGKAKWSSESYMDLCVGTILVAGCLLLSLSQT, (SEQ ID NO: 5013); XM_843679.5, SON, U_7, In, 37, essential, high expression, FQSEYCCSKANSTKKPGNINKRISCVIWISTSKKRSR, (SEQ ID NO: 5014); XM_014106286.1, MYCBP2, U_7, In, 37, high expression, SPGSNNARTRYAVLARRARHIQGSEDGTFRSQHQKLP, (SEQ ID NO: 5015); XM_005624915.2, GEMIN4, U_7, Del, 37, essential, FIPRYSVTFCTSWPCSPTRCASRCMSWPWKSSPAMKP, (SEQ ID NO: 5016); XM_014112649.1, RBPJ, U_7, Del, 38, essential, high expression, FALLLVCILWAVDGRKKKNKWNGMVVLNKSLNHVHLLE, (SEQ ID NO: 5017); XM_005622466.2, SOAT1, U_7, Del, 40, high expression, FMCTTSLKGSAPPCFGISNRSPSALAFWSYVYLTPSCQVC, (SEQ ID NO: 5019); XM_014118396.1, STK38, U_7, Del, 41, high expression, LKALTGNISERDLLQYLLKSKALMIPQTSMNFQNLIFLSQQ, (SEQ ID NO: 5021); XM_014110987.1, FN1, U_7, In, 41, high expression, SSILYSWLLRQWETLSDKPTMGAHLPGKCLGLYLLRREPRL, (SEQ ID NO: 5022); XM_014112366.1, MPHOSPH10, U_7, Del, 42, essential, high expression, LKTLILMKMKENCLEVKNLSQVKVPETSNTKTSLIQLKVMKT, (SEQ ID NO: 5023); XM_005636001.2, PPP1CC, U_7, Del, 42, high expression, FFSEETMNVPASIEFMDFTMNVKEDITLNYGKLSQTALTVYR, (SEQ ID NO: 5024); NM_001003033.1, PPP1CC, U_7, Del, 42, high expression, FFSEETMNVSASIEFMDFTMNVKEDITLNYGKLSQTALTVYR, (SEQ ID NO: 5025); XM_014109484.1, TMEM87A, U_7, In, 42, high expression, FHGDVYCICPVWCSVAGMVCLLLERSPENSVLDWCCHLLGNA, (SEQ ID NO: 5026); XM_014113785.1, ZCCHC2, U_7, Del, 42, high expression, FCLRLLMQTDWCMTQSWGAKPTTACSRWRDLGGSILCTQHRT, (SEQ ID NO: 5027); XM_005630848.2, SLC16A1, U_7, In, 42, essential, CSFYCCKWSVSSASTFILQLHWVLCLCGLLWICVWMAQLSVV, (SEQ ID NO: 5028); XM_859377.4, UPF3B, U_7, Del, 47, high expression, FLMILVCILICMPEHTSTLKTKRTLFCSGIALMVMYSLTIKVRNILL, (SEQ ID NO: 5030); XM_005620968.2, WBSCR22, U_7, In, 47, essential, FFALLCAGSWSPGRPAAIPGELRAVGANYDPGHQGWLHWWRGCGLPQ, (SEQ ID NO: 5031); XM_862279.4, G3BP1, U_7, Del, 48, high expression, FKVMGMWWSYALTVAGNYPILVLLCLMILSLFKRSLATGPSCSEVRSA, (SEQ ID NO: 5032); XM_014110041.1, PAPSS1, U_7, Del, 48, high expression, LKYLLMLLCMFVNRGMSKDSTKKHGLEKLKASLVSILSMKNQKLLNWC, (SEQ ID NO: 5033); XM_014118099.1, NOL8, U_7, Del, 48, essential, FLRMMKDLVVLIYSGEEWEVILAGILGRLEQTTCEWTVGRNIKMPKGE, (SEQ ID NO: 5034); XM_846061.4, CPB2, U_7, In, 49, high expression, CERIRCKQCESSFTRQHHPIQSLGGRSERSYPTANLQQHPQPPDLLLLL, (SEQ ID NO: 5035); XM_005616941.2, CUL2, U_7, Del, 54, essential, high expression, FWKITYGICTREFWSLKSKYLLCIIGIGRNIAKVQTIWTACIGISTLSLLKRIN, (SEQ ID NO: 5037); XM_003639382.3, DENND4C, U_7, Del, 54, high expression, LKLSENFSCLSTNFLCLDHILFPLKSIFHTSCKTSLFLHHKDQESLYSFQSMMH, (SEQ ID NO: 5038); XM_014118473.1, MRPL14, U_7, In, 57, essential, high expression, CWALGPLHSCKQSIQPAQFQHHWELQCNSEDDAGPCGGQQCPGEYSVPSPSSLHPCL, (SEQ ID NO: 5039); XM_005628322.1, SLC30A9, U_7, In, 57, essential, high expression, FPPRMAEFSDVEKLFTRDLLWASVPSRAESSEVVLHKRAERRTGVADSQSGKSTIVR, (SEQ ID NO: 5040); XM_535467.5, SLC12A1, U_7, In, 59, high expression, FSPSYWDSCWCQYLRRFGGSPRCHPQRNHAGHFHHHCRLYRSCYLCRGLCGPRCYRKHE, (SEQ ID NO: 5042); XM_014112420.1, TICRR, U_7, Del, 59, essential, LMLWILYYIRFTIHLKIQLPALLLFQSGPNRSLAALIPGMQLLWRSGFLPPTLVVPVQV, (SEQ ID NO: 5044); XM_014114799.1, ECT2L, U_7, In, 60, driver, FTMLQITIKVCPRLFRKDASGQTGFLYSVTTLFIFSIYLFLPESKRSVCSCTSQLALEVYN, (SEQ ID NO: 5045); XM_005639633.1, ATP13A3, U_7, Del, 61, high expression, FGSSSNLGMKYGIHNQMLVIQREAYIGILLHICTMKLNLIRVIYKIMKIPQCFLSPVFSTS, (SEQ ID NO: 5046); XM_014115004.1, TPR, U_7, Del, 64, driver, essential, high expression, LMMKTELFQVLQLLWFHIVLMDLLKQFIPHKLLVFLDSGLGHLKICHKQVQVTLILANLLLKEA, (SEQ ID NO: 5047); XM_544475.5, NUDC, U_7, Del, 64, essential, high expression, LLEEKRGWQRSSSHRPSTTITSWHRRPDGRRELGRRLSGGRRPRGQPGWPKRPNQRPLGPRSRS, (SEQ ID NO: 5048); XM_005627278.2, STK38, U_7, Del, 68, high expression, LKALTGNISERDLLQYLLKSKALMIPQTSMNFQNLIFLSQQLQVITLRLTTRTKTGSSSITHTSALRA, (SEQ ID NO: 5049); XM_005618818.2, SPRTN, U_7, Del, 68, essential, LSRKSKYKVVEMIQSGVHILQLQLRIPAVHPARTGWSIVLFVRMKCWSLRLMSTWTGALKVVASK SKV, (SEQ ID NO: 5050); XM_014118114.1, SENP6, U_7, Del, 71, essential, high expression, LQKFHLKKPIADLLPVQEPMKRASKEVVCKKKTKLKMCPLNLKYNLKTNKNSSFLMMMKKLERH PSSWAL, (SEQ ID NO: 5053); XM_005633841.2, ALG11, U_7, In, 76, essential, high expression, LFIIPPWANSLWNFMCMSVRYPLGNQTAATEKERVSINQQKWEEANGGCIFSSILQCRWRRRKSFV VCLKGPAEKV, (SEQ ID NO: 5054); XM_005624856.2, KIAA0100, U_7, Del, 77, essential, high expression, FGSRMSALSFSNTSKLWKLIACGFPANSLAMICHAMWHCALEKYVSGRTYRRFLAFLPHFPKALGY IKRNCLSVHPY, (SEQ ID NO: 5055); XM_003433052.3, ALG11, U_7, In, 77, essential, high expression, LFIIPPWANSLWNFMCMSVRYPLGNQTAATEKERVSINQQKWEEANGGCIFSSILQCRWRRRKSFV VCLKGPAEKVP, (SEQ ID NO: 5056); XM_542156.5, CHAF1A, U_7, Del, 88, essential, FSKGRCLWWSCRISWLRNLLKPSLQRHLHTRACPLRVRCWSLALKKTLFLATHPGVLPLLPAHLRG SLLPKSSTAVPGPSPPPHLSAE, (SEQ ID NO: 5057); XM_003640168.3, ABT1, U_7, In, 89, essential, FPARGRVREAQEEGSVSLSHGREKAVQVQQGLIARGLGGVPGQASSQACGGQSSQHAHGSPKAQPL PLRPMESQVPAPFHVVPPQRTSCL, (SEQ ID NO: 5058); XM_547724.5, SLC7A7, U_7, In, 91, high expression, FPHGLLALDHLPGGRGTLQGYHQIPHRDWDWPFRVGPLLPQQQGGKKQKNSLGPKNCSIHYMVPP GPMYVSCCRDGFGRWRRDAQATRSQV, (SEQ ID NO: 5059); XM_005630266.2, NRBP1, C_7, In, 8, essential, high expression, HHPWEPDL, (SEQ ID NO: 5060); XM_548413.5, ABL1, C_7, In, 8, driver, high expression, PTPAGEKD, (SEQ ID NO: 5061); XM_848227.4, C1S, C_7, Del, 8, high expression, PNTSSMKI, (SEQ ID NO: 5062); XM_014107801.1, CCDC92, C_7, In, 8, high expression, HRLRPQLK, (SEQ ID NO: 5063); XM_854764.4, MAP1A, C_7, In, 8, high expression, QHGGCPFG, (SEQ ID NO: 5064); XM_005640267.2, XIRP2, C_7, In, 8, high expression, RGPAAPCP, (SEQ ID NO: 5065); XM_543631.5, CALCOCO1, C_7, In, 8, high expression, PVEGVPYL, (SEQ ID NO: 5066); XM_539966.5, PLEKHA2, C_7, Del, 8, high expression, PSLRVATV, (SEQ ID NO: 5067); XM_005622562.1, RCSD1, C_7, In, 8, high expression, QGRAGPEW, (SEQ ID NO: 5068); XM_537502.5, SIPA1L1, C_7, Del, 8, high expression, PEVKVLTI, (SEQ ID NO: 5069); NM_001013847.1, TPP1, C_7, In, 8, high expression, HIIPEATP, (SEQ ID NO: 5070); XM_014110912.1, BARD1, C_7, In, 8, essential, PSSGGSAP, (SEQ ID NO: 5071); XM_005616873.2, CD3EAP, C_7, Del, 8, essential, PLRCRNPW, (SEQ ID NO: 5072); XM_545182.4, ICE1, C_7, Del, 8, essential, PLWYPYHP, (SEQ ID NO: 5073); XM_005619042.2, ZSWIM8, C_7, Del, 8, essential, PKGPTTWL, (SEQ ID NO: 5074); XM_014119889.1, BRAF, C_7, In, 9, driver, essential, high expression, PCLIAWLTH, (SEQ ID NO: 5075); XM_005642083.2, USP9X, C_7, In, 9, essential, high expression, PPTESDFIA, (SEQ ID NO: 5076); XM_848382.3, EBNA1BP2, C_7, In, 9, essential, high expression, PAVGFGLGI, (SEQ ID NO: 5077); XM_014106445.1, COL4A2, C_7, Del, 9, high expression, PDPQALQDK, (SEQ ID NO: 5078); XM_014106445.1, COL4A2, C_7, In, 9, high expression, PFQHVWVTW, (SEQ ID NO: 5079); XM_014122069.1, KIAA1462, C_7, Del, 9, high expression, PLGEQTPTA, (SEQ ID NO: 5080); XM_014116817.1, MYO18A, C_7, In, 9, high expression, PEDLPGAQG, (SEQ ID NO: 5081); XM_846233.4, PPFIA1, C_7, Del, 9, high expression, PLPPQDRSA, (SEQ ID NO: 5082); XM_003639958.3, ATN1, C_7, Del, 9, high expression, PHLLPIAGS, (SEQ ID NO: 5083); XM_843925.4, DAP, C_7, Del, 9, high expression, PSRPCSSQE, (SEQ ID NO: 5084); XM_843647.4, PDLIM3, C_7, In, 9, high expression, PVRCVPDAP, (SEQ ID NO: 5085); XM_544610.5, THBS1, C_7, Del, 9, high expression, PCLMTSSKT, (SEQ ID NO: 5086); XM_014112559.1, TNIP2, C_7, In, 9, high expression, PRAREDLLP, (SEQ ID NO: 5087); XM_014108635.1, ZNF641, C_7, In, 9, high expression, PSAKVPCVH, (SEQ ID NO: 5088); XM_005620305.1, KMT2A, C_7, In, 9, driver, essential, PEPTWLSQG, (SEQ ID NO: 5089); XM_533642.4, CLASRP, C_7, In, 9, essential, PARRPRTGT, (SEQ ID NO: 5090); XM_014122593.1, MCM10, C_7, In, 9, essential, RSCSQSGEN, (SEQ ID NO: 5091); XM_547651.5, THOC1, C_7, Del, 9, essential, PMEKDFQRW, (SEQ ID NO: 5092); XM_014109365.1, CDAN1, C_7, In, 9, essential, RPCRDEAFT, (SEQ ID NO: 5093); XM_014117336.1, MKL1, C_7, In, 9, driver, PDTQPLGSQ, (SEQ ID NO: 5094); XM_845621.5, MLLT6, C_7, Del, 9, driver, PHCCLPGPW, (SEQ ID NO: 5095); XM_014109357.1, NUTM1, C_7, Del, 9, driver, PLRVLPVNS, (SEQ ID NO: 5096); XM_014106883.1, PTK6, C_7, Del, 9, driver, PSAHPPHTS, (SEQ ID NO: 5097); XM_014111168.1, SPEN, C_7, Del, 10, driver, essential, high expression, PLRWSLRSLF, (SEQ ID NO: 5098); XM_005621271.2, RNF40, C_7, Del, 10, essential, high expression, PSVSQLWLLW, (SEQ ID NO: 5099); XM_014118847.1, ANKRD17, C_7, Del, 10, high expression, PQCPLREIPL, (SEQ ID NO: 5100); XM_005637802.2, TACC2, C_7, Del, 10, high expression, PRGRKLLSGP, (SEQ ID NO: 5101); XM_003639958.3, ATN1, C_7, In, 10, high expression, PNEVSIFVLW, (SEQ ID NO: 5102); XM_014115482.1, DTNA, C_7, In, 10, high expression, PVSGLAAPSA, (SEQ ID NO: 5103); XM_005618546.2, LDB2, C_7, In, 10, high expression, RPGEQQPLEQ, (SEQ ID NO: 5104); XM_854174.4, BAG6, C_7, Del, 10, essential, PRHQLWPRSR, (SEQ ID NO: 5105); XM_014119446.1, KTI12, C_7, In, 10, essential, RAYGSPEGAL, (SEQ ID NO: 5106); XM_859725.4, WAPL, C_7, Del, 10, essential, PTENMKQVSK, (SEQ ID NO: 5107); XM_005619042.2, ZSWIM8, C_7, In, 10, essential, QRGLQRGCDV, (SEQ ID NO: 5108); XM_014119889.1, BRAF, C_7, Del, 11, driver, essential, high expression, PLPHCLAHSLM, (SEQ ID NO: 5109); XM_014120337.1, BIRC6, C_7, Del, 11, essential, high expression, PLQCSVTTDCP, (SEQ ID NO: 5110); XM_005631964.2, ZEB2, C_7, Del, 11, essential, high expression, PRKTLYYPGRP, (SEQ ID NO: 5111); XM_014106445.1, COL4A2, C_7, In, 11, high expression, RNHRVSWVHRK, (SEQ ID NO: 5112); XM_014106445.1, COL4A2, C_7, In, 11, high expression, RTPRHSRTNRL, (SEQ ID NO: 5113); XM_853562.3, R3HDM2, C_7, In, 11, high expression, PEPIHGPVESL, (SEQ ID NO: 5114); XM_005637802.2, TACC2, C_7, In, 11, high expression, PGEGSFYLGPR, (SEQ ID NO: 5115); XM_537756.4, CPD, C_7, In, 11, high expression, RGQWSRQQPRP, (SEQ ID NO: 5116); XM_536288.4, PJA2, C_7, Del, 11, high expression, PLPVTARRMPP, (SEQ ID NO: 5117); XM_536262.5, TBC1D1, C_7, In, 11, high expression, PGARGRPLLGR, (SEQ ID NO: 5118); XM_014112559.1, TNIP2, C_7, Del, 11, high expression, PQSPRRPPALS, (SEQ ID NO: 5119); XM_005616171.2, TNNT1, C_7, In, 11, high expression, QDPRRGTCGLR, (SEQ ID NO: 5120); XM_849540.4, XBP1, C_7, Del, 11, high expression, PRRASDSASRT, (SEQ ID NO: 5121); XM_847443.4, GPN2, C_7, Del, 11, essential, PARARPRTAWA, (SEQ ID NO: 5122); XM_014107603.1, HJURP, C_7, In, 11, essential, PRTLGCGHVQG, (SEQ ID NO: 5123); XM_005630474.2, POLR1A, C_7, In, 11, essential, PDGVWQITALL, (SEQ ID NO: 5124); XM_005621293.2, SRCAP, C_7, In, 11, essential, PSHKPPLGLGA, (SEQ ID NO: 5125); XM_533370.5, ZNF407, C_7, In, 11, essential, PWRGEASLPGL, (SEQ ID NO: 5126); XM_005616464.2, CBLC, C_7, In, 11, driver, PTTPSAECVRS, (SEQ ID NO: 5127); XM_014108123.1, LZTR1, C_7, In, 11, driver, PAALWAHHGGL, (SEQ ID NO: 5128); XM_005635182.1, NFATC2, C_7, Del, 11, driver, PRPALPSSWTP, (SEQ ID NO: 5129); XM_014117863.1, PAXS, C_7, Del, 11, driver, PPDRAATQHRR, (SEQ ID NO: 5130); XM_014109523.1, PML, C_7, Del, 11, driver, PRASELSACRP, (SEQ ID NO: 5131); XM_860093.4, EWSR1, C_7, In, 12, driver, essential, high expression, PNWILQPGSKSI, (SEQ ID NO: 5132); XM_014120337.1, BIRC6, C_7, In, 12, essential, high expression, PSNAVSPQTVHD, (SEQ ID NO: 5133); XM_014120153.1, FAT1, C_7, In, 12, driver, high expression, QIHHCKSNRENP, (SEQ ID NO: 5134); XM_544279.5, KLF6, C_7, In, 12, driver, high expression, REGARRDCGEAR, (SEQ ID NO: 5135); XM_858879.4, EPHX1, C_7, Del, 12, high expression, PSCRPAAPQSPC, (SEQ ID NO: 5136); XM_014121888.1, MAP4, C_7, In, 12, high expression, HTCAQEAHCCQD, (SEQ ID NO: 5137); XM_005630633.2, HIPK1, C_7, In, 12, high expression, PTPAATRTGRWS, (SEQ ID NO: 5138); XM_014119781.1, HIPK2, C_7, In, 12, high expression, PENPGQRSAGGM, (SEQ ID NO: 5139); XM_005636665.1, PAPSS2, C_7, Del, 12, high expression, PTASWRPRPGRC, (SEQ ID NO: 5140); XM_847680.4, CHERP, C_7, In, 12, essential, PHAATGPSPHQP, (SEQ ID NO: 5141); XM_847443.4, GPN2, C_7, In, 12, essential, RLGQDHVLPGHE, (SEQ ID NO: 5142); XM_005629703.2, PAXIP1, C_7, Del, 12, essential, PEESHAPSTLFL, (SEQ ID NO: 5143); XM_003433467.3, TARBP2, C_7, In, 12, essential, HGGAAPRLPSAV, (SEQ ID NO: 5144); XM_546181.5, ZMIZ1, C_7, Del, 12, essential, PGNSPCSPPSTL, (SEQ ID NO: 5145); XM_014111713.1, ATP2B3, C_7, Del, 12, driver, PRPRPPTRTTTP, (SEQ ID NO: 5146); NM_001287125.1, CDH1, C_7, In, 12, driver, HLQPNHVPGTGA, (SEQ ID NO: 5147); XM_005626284.2, FLT4, C_7, In, 12, driver, PNSKHHGGDTHH, (SEQ ID NO: 5148); XM_844902.3, IL21R, C_7, In, 12, driver, PFQRDCDLLGTL, (SEQ ID NO: 5149); XM_014117336.1, MKL1, C_7, In, 12, driver, LTHGHLGIALCP, (SEQ ID NO: 5150); XM_005625433.1, NOTCH1, C_7, Del, 12, driver, PRSAPPMATWAT, (SEQ ID NO: 5151); XM_005638000.2, PLAG1, C_7, Del, 12, driver, PRHRIFRILQTP, (SEQ ID NO: 5152); NM_001110767.1, MYH9, C_7, Del, 13, driver, essential, high expression, PTSMPSQTPPTGV, (SEQ ID NO: 5153); XM_005637906.2, TUBGCP2, C_7, Del, 13, essential, high expression, PQHQRPGSPSLPS, (SEQ ID NO: 5154); XM_005637906.2, TUBGCP2, C_7, In, 13, essential, high expression, PSISAQGRRPCPV, (SEQ ID NO: 5155); XM_005636789.2, EIF4B, C_7, In, 13, essential, high expression, PKTQTESKASEYS, (SEQ ID NO: 5156); XM_005630688.2, NOTCH2, C_7, In, 13, driver, high expression, PCPSPCSACTVLL, (SEQ ID NO: 5157); NM_001122602.1, IGF2R, C_7, In, 13, high expression, RHCCLPGERGAGV, (SEQ ID NO: 5158); XM_545954.4, KLF3, C_7, Del, 13, high expression, PRKHCCKRIIHQS, (SEQ ID NO: 5159); XM_014106444.1, COL4A1, C_7, In, 13, high expression, RSSRYPWVQWNKG, (SEQ ID NO: 5160); XM_005631208.2, TNKS1BP1, C_7, Del, 13, high expression, PGVPWLSCLLPGR, (SEQ ID NO: 5161); XM_846534.4, PRCC, C_7, Del, 13, driver, essential, PWAFSPQRRGRSP, (SEQ ID NO: 5162); XM_537028.5, WDR77, C_7, In, 13, essential, PGAPRGPRVEPPP, (SEQ ID NO: 5163); XM_543052.4, MOCS3, C_7, Del, 13, essential, PKLRGHALVYSSR, (SEQ ID NO: 5164); XM_541589.4, CIC, C_7, Del, 13, driver, PHPPAPPQLPQAG, (SEQ ID NO: 5165); XM_541589.4, CIC, C_7, In, 13, driver, PTPRPLHSCHRQV, (SEQ ID NO: 5166); XM_005637736.2, TCF7L2, C_7, Del, 13, driver, PQTSLSALKSSGT, (SEQ ID NO: 5167); XM_014113364.1, ARHGEF12, C_7, In, 14, driver, high expression, LLCRLTKPDHGVHS, (SEQ ID NO: 5168); XM_005621365.2, ATXN2L, C_7, Del, 14, high expression, PSARTNTSQPQPLR, (SEQ ID NO: 5169); XM_844466.4, MAZ, C_7, In, 14, high expression, PCPGRRPPATAHLL, (SEQ ID NO: 5170); XM_844466.4, MAZ, C_7, Del, 14, high expression, PLSRETPPSHSPPV, (SEQ ID NO: 5171); XM_014113911.1, RERE, C_7, Del, 14, high expression, PLRAPTLLLLSTRA, (SEQ ID NO: 5172); XM_003431676.3, CFB, C_7, Del, 14, high expression, PQQTGTVPATLSSL, (SEQ ID NO: 5173); XM_547871.5, DCAFS, C_7, In, 14, high expression, PKQARQYLHWRRQL, (SEQ ID NO: 5174); XM_005630632.1, HIPK1, C_7, Del, 14, high expression, PHQCRRVPSAVRRN, (SEQ ID NO: 5175); XM_014112392.1, MEF2A, C_7, Del, 14, high expression, PSPDRKWGAPLWTV, (SEQ ID NO: 5176); XM_548250.5, ACACA, C_7, In, 14, essential, PAFRHADIYGARVG, (SEQ ID NO: 5177); XM_856636.4, BRPF1, C_7, In, 14, essential, PAPHHGLPTSAQAG, (SEQ ID NO: 5178); XM_014114273.1, CPSF4, C_7, Del, 14, essential, PRWEPVWALLGSMR, (SEQ ID NO: 5179); XM_005618689.2, GAK, C_7, In, 14, essential, PGQAAPQGLSAAKA, (SEQ ID NO: 5180); XM_546181.5, ZMIZ1, C_7, In, 14, essential, PQLYELHETHSVAQ, (SEQ ID NO: 5181); XM_014114088.1, AKT2, C_7, In, 14, driver, PEQLLCRRMPADED, (SEQ ID NO: 5182); NM_001003016.2, MCL1, C_7, In, 15, essential, high expression, PEAAAARAPLPRVAA, (SEQ ID NO: 5183); XM_847340.4, UBAP1, C_7, In, 15, essential, high expression, HSCSKQYQISVLPQT, (SEQ ID NO: 5184); XM_014113410.1, ZBTB16, C_7, In, 15, driver, high expression, RLEDREDLPLPVLRV, (SEQ ID NO: 5185); XM_014118847.1, ANKRD17, C_7, In, 15, high expression, PSALFERYRFNHSSR, (SEQ ID NO: 5186); XM_014116831.1, ABR, C_7, In, 15, high expression, HFLRRTEAEHTVLLH, (SEQ ID NO: 5187); XM_014114672.1, HCFC1R1, C_7, In, 15, high expression, HGIPPISAPTQEPVL, (SEQ ID NO: 5188); XM_005640837.2, MIA3, C_7, In, 15, high expression, PGRLGWHVGRDAATP, (SEQ ID NO: 5189); XM_543139.5, PDS5B, C_7, In, 15, high expression, PPSQIRHSLYPCDIF, (SEQ ID NO: 5190); XM_003431676.3, CFB, C_7, In, 15, high expression, PSRLEPYPPRYHPYD, (SEQ ID NO: 5191); NM_001313776.1, CKM, C_7, Del, 15, high expression, PTAPGASAGQWRNSP, (SEQ ID NO: 5192); XM_541508.4, PLEKHA4, C_7, Del, 15, high expression, PWTQLFTKAWRQILC, (SEQ ID NO: 5193); XM_536262.5, TBC1D1, C_7, In, 15, high expression, PGGPPGARGRPLLGR, (SEQ ID NO: 5194); XM_547530.4, ISG20L2, C_7, Del, 15, essential, PSTGRLTAQRMPLCL, (SEQ ID NO: 5195); XM_861323.3, SRSF10, C_7, In, 15, essential, QHLSVRKERGRRHQV, (SEQ ID NO: 5196); XM_547651.5, THOC1, C_7, In, 15, essential, RWRKIFKDGRAYIKH, (SEQ ID NO: 5197); XM_014117542.1, BCL11A, C_7, Del, 15, driver, PCLAHHRDITWTPTA, (SEQ ID NO: 5198); XM_005617744.2, ARID1A, C_7, In, 16, driver, essential, high expression, LARHEEQFGWDPGCLS, (SEQ ID NO: 5199); XM_854764.4, MAP1A, C_7, In, 16, high expression, PCSYPPEQRPKPPSEW, (SEQ ID NO: 5200); XM_854764.4, MAP1A, C_7, Del, 16, high expression, PSFILHCWEMGSTLLV, (SEQ ID NO: 5201); XM_014112907.1, SEC24C, C_7, In, 16, high expression, PVFSTSGSYWQTCRCL, (SEQ ID NO: 5203); XM_014107674.1, TRAF3IP1, C_7, Del, 16, high expression, PRSLRCQNLKWCPRWT, (SEQ ID NO: 5204); XM_014109945.1, WDFY3, C_7, Del, 16, high expression, PRGPQVTRSFSITWTT, (SEQ ID NO: 5205); XM_014113507.1, ARRB2, C_7, Del, 16, high expression, PGPTHPSPPLACRTGC, (SEQ ID NO: 5206); XM_005629454.1, TMEM192, C_7, In, 16, high expression, PRLPENPSCRAPPPRS, (SEQ ID NO: 5207); XM_014111079.1, UBR4, C_7, Del, 16, essential, PCWTSHLMQMTRPWLN, (SEQ ID NO: 5209); XM_014109365.1, CDAN1, C_7, Del, 16, essential, PALQGRSLHVGSTQLR, (SEQ ID NO: 5210); XM_005618685.2, GAK, C_7, Del, 16, essential, PMTPGTLCSTTSSAPR, (SEQ ID NO: 5211); XM_005616531.1, CIC, C_7, In, 16, driver, PHFGGGGTWKGSPSRH, (SEQ ID NO: 5212); XM_014119335.1, MYCL, C_7, In, 16, driver, RRKRGSPVLPPQTWQF, (SEQ ID NO: 5213); XM_005617746.2, ARID1A, C_7, In, 17, driver, essential, high expression, LARHEQEQFGWDPGCLS, (SEQ ID NO: 5214); XM_855027.4, ATP2A1, C_7, In, 17, high expression, PEARGDDSGRLCQVHGI, (SEQ ID NO: 5215); XM_847792.4, IGFBP5, C_7, Del, 17, high expression, PAPWAASWSKSPAAVAA, (SEQ ID NO: 5216); XM_544699.6, CGNL1, C_7, Del, 17, high expression, PQPRIKRRTKSRQPRRR, (SEQ ID NO: 5217); XM_844754.4, PLEKHO1, C_7, Del, 17, high expression, PPSFRPTRPSCPGSRTW, (SEQ ID NO: 5218); XM_005623705.2, TMED8, C_7, In, 17, high expression, PSDGASMHLDLCQDERI, (SEQ ID NO: 5219); XM_014122593.1, MCM10, C_7, Del, 17, essential, PLLLPVRGKLIKSCKMN, (SEQ ID NO: 5220); XM_005620578.2, ZC3H18, C_7, In, 17, essential, QEADRQWQRQRQQLQRL, (SEQ ID NO: 5221); XM_014118655.1, ZC3H3, C_7, In, 17, essential, PRGQTSSRIQQFEAPLQ, (SEQ ID NO: 5222); XM_862165.4, ATP2B3, C_7, Del, 17, driver, PSSPRPSCSWCGRPCRT, (SEQ ID NO: 5223); XM_014109357.1, NUTM1, C_7, In, 17, driver, PASSILCREPSGALCSP, (SEQ ID NO: 5224); XM_536182.5, AKAP13, C_7, Del, 18, high expression, PLPSAVKVHRLMQPPLAL, (SEQ ID NO: 5225); XM_014113005.1, FAF2, C_7, Del, 18, high expression, PHFRRPDSATQKFSLFRT, (SEQ ID NO: 5226); XM_014115267.1, ENAH, C_7, Del, 18, high expression, PSLHLDFLRHPRPKTIAL, (SEQ ID NO: 5228); XM_014109253.1, PCMTD1, C_7, In, 18, high expression, LCCQESTGLGSYLYSTYT, (SEQ ID NO: 5229); XM_849952.3, INTS5, C_7, Del, 18, essential, PLTSVHPHPPMSLLGDLV, (SEQ ID NO: 5230); XM_005629703.2, PAXIP1, C_7, In, 18, essential, RRKAMLPAHYFCDWICRQ, (SEQ ID NO: 5231); XM_014117863.1, PAXS, C_7, In, 18, driver, RRTGQLLSTDADRDGAWE, (SEQ ID NO: 5232); XM_852361.4, TCF20, C_7, In, 19, high expression, LAEQDSERQPQHRAVGAGV, (SEQ ID NO: 5234); XM_852361.4, TCF20, C_7, Del, 19, high expression, PCRTRQRKAASAQSSRSGG, (SEQ ID NO: 5235); XM_005620011.2, VAMP2, C_7, In, 19, high expression, PCRPGWGGRPPGAPSKPHQ, (SEQ ID NO: 5236); XM_014116993.1, NUP214, C_7, In, 19, driver, essential, PISNNCHPPSYRIPHIVLW, (SEQ ID NO: 5237); XM_533117.5, FDXR, C_7, Del, 19, essential, PRSVWWAVDPLASIPPNTC, (SEQ ID NO: 5238); XM_005632488.2, VPRBP, C_7, In, 19, essential, PEFTSFAWTILCRQFPLDW, (SEQ ID NO: 5239); XM_535546.6, SIN3A, C_7, Del, 20, essential, high expression, PLCMKQCLRPCSQLREFSTL, (SEQ ID NO: 5240); XM_014113364.1, ARHGEF12, C_7, Del, 20, driver, high expression, PLVQTHKARSWSTFVRSRLT, (SEQ ID NO: 5241); XM_005615653.2, AKAP7, C_7, Del, 20, high expression, PAPQALSAATQQATTAPPGA, (SEQ ID NO: 5242); XM_005635260.2, OSBPL2, C_7, In, 20, high expression, PREWDSETQDVIARPDVYQK, (SEQ ID NO: 5243); XM_535042.5, TACC2, C_7, In, 20, high expression, PSERDTTRARGRVSRPQRGD, (SEQ ID NO: 5245); XM_531794.5, TMEM131, C_7, Del, 20, high expression, PHWKVNSAEISLPRFLFQLH, (SEQ ID NO: 5246); XM_014113005.1, FAF2, C_7, In, 20, high expression, HTSGGRTQPHRSSLCSGPDR, (SEQ ID NO: 5247); XM_005629518.1, EZH2, C_7, Del, 20, driver, essential, PQGRRRGNTGCGRHTAERYS, (SEQ ID NO: 5248); XM_014110912.1, BARD1, C_7, Del, 20, essential, PLLGWERSLGITLRAKFFGL, (SEQ ID NO: 5249); XM_005632733.2, HAUS8, C_7, Del, 20, essential, PLTWTSRPSMTKVCLERLRN, (SEQ ID NO: 5250); XM_005627027.2, MAPK14, C_7, Del, 20, essential, PLLISLTGCQAMRQETTFSL, (SEQ ID NO: 5251); XM_014114423.1, POLR3E, C_7, Del, 20, essential, PRLLHPQMSRRCSPSGSLET, (SEQ ID NO: 5252); XM_854174.4, BAG6, C_7, In, 20, essential, PDTSCGLGAGSLELSNIRSS, (SEQ ID NO: 5253); XM_005618689.2, GAK, C_7, Del, 20, essential, PRPSRPPRPVRSQGLTMLPT, (SEQ ID NO: 5254); XM_005639752.2, SOX2, C_7, Del, 20, driver, PWLPPPPTPGRPARPGTSGT, (SEQ ID NO: 5255); XM_014122282.1, TCF3, C_7, In, 20, driver, PDPVQPGRGRGRPSPPRAEG, (SEQ ID NO: 5256); XM_014113682.1, PTPRK, C_7, Del, 21, driver, high expression, PVLALSWCIAVLVLDGLAVTS, (SEQ ID NO: 5257); XM_537251.5, ASH1L, C_7, In, 21, high expression, PTSSSFLYACWPFTSQSNQIP, (SEQ ID NO: 5258); XM_014112562.1, FAM193A, C_7, In, 21, high expression, PTPHHRWLHQRSTQRLQRPRL, (SEQ ID NO: 5259); XM_854764.4, MAP1A, C_7, In, 21, high expression, LLSFYIVGRWEALSWCDHKPW, (SEQ ID NO: 5260); XM_014111003.1, MAP2, C_7, Del, 21, high expression, PRSGRISPRPRWRLRVSSLAP, (SEQ ID NO: 5261); XM_844054.5, BNIP3, C_7, Del, 21, high expression, PRSSCSSTRSARPRSACGTQA, (SEQ ID NO: 5262); XM_005622656.2, CAPN2, C_7, Del, 21, high expression, PLTCSGSSRRLCRKAPSSAAP, (SEQ ID NO: 5263); XM_014112392.1, MEF2A, C_7, In, 21, high expression, PAPTGNGALPCGQSEQLKQLL, (SEQ ID NO: 5264); XM_014116993.1, NUP214, C_7, In, 21, driver, essential, PSEIHCSSQPVPVSLPVTEIL, (SEQ ID NO: 5265); XM_014112349.1, BOD1L1, C_7, Del, 21, essential, PEPGLEASRGWKKPLRRRPSG, (SEQ ID NO: 5266); XM_014112560.1, FAM193A, C_7, In, 22, high expression, RHWRHPQSPAAPPSGFCSRLSL, (SEQ ID NO: 5268); XM_005640629.2, TNS1, C_7, Del, 22, high expression, PRVLALAAGPSTPAWLPPGVPA, (SEQ ID NO: 5269); XM_005642194.1, AEBP1, C_7, In, 22, high expression, PYSGLLLRASQTQETPHGPGDR, (SEQ ID NO: 5270); XM_845916.4, COL3A1, C_7, In, 22, high expression, RSGRAPRRSRACGSPRSPGCQR, (SEQ ID NO: 5271); XM_536455.5, CYFIP2, C_7, Del, 22, high expression, PTSRPSADSWVTRASQWSWRNC, (SEQ ID NO: 5272); XM_535696.4, ENPEP, C_7, In, 22, high expression, LAGPFARDPAARGPGGLSRQRR, (SEQ ID NO: 5273); XM_533751.4, LMCD1, C_7, Del, 22, high expression, PLESPRNWVCSTWSSSQKRSSQ, (SEQ ID NO: 5274); XM_014109253.1, PCMTD1, C_7, Del, 22, high expression, PVLSGIYRTWLVFIFDVHLEIS, (SEQ ID NO: 5275); XM_541508.4, PLEKHA4, C_7, In, 22, high expression, PGPDPGGGGVASASHAAAVQLL, (SEQ ID NO: 5276); XM_846492.4, XIRP1, C_7, In, 22, high expression, RTTGGSPSPKREAHSWFPARGP, (SEQ ID NO: 5277); XM_014108635.1, ZNF641, C_7, Del, 22, high expression, PQCQSAMCALSVGRALADGTTW, (SEQ ID NO: 5278); XM_014116993.1, NUP214, C_7, Del, 22, driver, essential, PHQQQLPPPFLPHSPHCPLVAS, (SEQ ID NO: 5279); XM_014121534.1, VHL, C_7, In, 22, driver, essential, LRRRGPPSSRGAASACWSPPSL, (SEQ ID NO: 5280); XM_014107603.1, HJURP, C_7, Del, 22, essential, PQDPRLWPCTGVTQRALAAPRP, (SEQ ID NO: 5281); XM_014118288.1, LENG8, C_7, Del, 22, essential, PLSSCQQPSPLSPPTPRMGLML, (SEQ ID NO: 5282); XM_005638369.2, SPATA5L1, C_7, Del, 22, essential, PEWARPSWCGRWPGKPARSCWP, (SEQ ID NO: 5283); XM_005618689.2, GAK, C_7, In, 22, essential, PCAGQWGSPQHRRPAQPPPGGP, (SEQ ID NO: 5284); XM_541488.5, PNKP, C_7, Del, 22, essential, PGEPPPSSCPRTGKPLSWAGDP, (SEQ ID NO: 5285); XM_546181.5, ZMIZ1, C_7, In, 22, essential, RETPHAAPPQLYELHETHSVAQ, (SEQ ID NO: 5286); XM_014109357.1, NUTM1, C_7, Del, 22, driver, PCLQHSLQGTLWCSLLSLAHCW, (SEQ ID NO: 5287); XM_005639159.2, AFF1, C_7, In, 23, driver, high expression, PVICTFQPASSSPDTRKQQDSQQ, (SEQ ID NO: 5288); XM_005635260.2, OSBPL2, C_7, Del, 23, high expression, PSVHFIQKASIRTSCFTAPSTRS, (SEQ ID NO: 5289); XM_003434001.4, COL6A1, C_7, In, 23, high expression, RDKRHQRLPRPQGRRGRSRRPRR, (SEQ ID NO: 5290); XM_532844.5, SLC25A4, C_7, In, 23, high expression, PPTPTGPACQQTDQCREAVQRDH, (SEQ ID NO: 5291); XM_537807.5, GTF3C5, C_7, In, 23, essential, RLGREPDWPEQGPAPPQCHLCQL, (SEQ ID NO: 5292); XM_014112912.1, ARID1B, C_7, Del, 23, driver, PSHPPSEGRSPFLLAQSRRRSQS, (SEQ ID NO: 5293); XM_014114743.1, AXIN1, C_7, In, 23, driver, PLRRRGLRGTAGRTRGEPREHPG, (SEQ ID NO: 5294); XM_852751.4, POLR2A, C_7, Del, 24, essential, high expression, PPGTAHARCAPSSGCSSESSVRMN, (SEQ ID NO: 5295); XM_005622278.1, PTPRC, C_7, In, 24, driver, high expression, PARTQQLRGASARLQHPGQCQLLR, (SEQ ID NO: 5296); XM_014121877.1, MAP4, C_7, In, 24, high expression, RWGGASHPRGSARSWRPHFSQWPQ, (SEQ ID NO: 5297); XM_014112966.1, TULP4, C_7, Del, 24, high expression, PAFALTSPPPSSLPSSPQFQIPTT, (SEQ ID NO: 5298); XM_005636665.1, PAPSS2, C_7, In, 24, high expression, RRLHGAQGLEGADRLLRLPGEARL, (SEQ ID NO: 5299); XM_005620578.2, ZC3H18, C_7, Del, 24, essential, PGGGPSVAAAAAAATAALAPDPGP, (SEQ ID NO: 5300); XM_005632682.2, CRTC1, C_7, In, 24, driver, PREPGPAANGDRHYLGAGSAAVPC, (SEQ ID NO: 5301); XM_005617639.1, LCK, C_7, In, 24, driver, PRHQADYQQTLGHGSPNCRGHGIH, (SEQ ID NO: 5302); XM_005624339.1, RARA, C_7, Del, 24, driver, PHPAAVALASAPAQTEAARPPTPR, (SEQ ID NO: 5303); XM_014107007.1, DIDO1, C_7, Del, 25, essential, high expression, PRGLPHRHPRCRSHPVRLRHRHRQC, (SEQ ID NO: 5304); XM_844246.4, RBM17, C_7, In, 25, essential, high expression, PACGGRPEGSCSQWIFCRRSFDSFS, (SEQ ID NO: 5305); XM_005624459.2, STAT3, C_7, In, 25, driver, high expression, QHLPRSPRKLDNLISRVTTSDPPTN, (SEQ ID NO: 5306); XM_858879.4, EPHX1, C_7, In, 25, high expression, PAAVQQHPKALADGTRLAWLLLRVL, (SEQ ID NO: 5307); XM_005617055.2, KIAA1462, C_7, In, 25, high expression, PSESRPRPPDEDEGDSYDPSRVERV, (SEQ ID NO: 5308); XM_536899.5, MYLPF, C_7, Del, 25, high expression, PTWEATWTTRTFATSSRMATPRTRS, (SEQ ID NO: 5309); XM_536899.5, MYLPF, C_7, In, 25, high expression, RRGRQRGLQEHLLRHHAWRRQGPGV, (SEQ ID NO: 5310); XM_014115267.1, ENAH, C_7, In, 25, high expression, PPRASTTPSAPCFRTSTTTPSTPSS, (SEQ ID NO: 5311); XM_014108204.1, CABIN1, C_7, In, 25, essential, RQGQVAPPAQHAKAGHPLGRHQVPP, (SEQ ID NO: 5312); XM_532351.5, CYC1, C_7, In, 25, essential, QLPVVPPRPPVFPGPHQHPEGLPGI, (SEQ ID NO: 5313); XM_014112439.1, FURIN, C_7, Del, 25, essential, PRGNASLTSSLNPRTSGSGWRCGRL, (SEQ ID NO: 5314); XM_003639850.3, VPS16, C_7, Del, 25, essential, PRSRWSGAAVLAAKRGLSWWHGRGV, (SEQ ID NO: 5316); NM_001287125.1, CDH1, C_7, Del, 25, driver, PSSTQPRTRDGCLRTRLTSKSLYSK, (SEQ ID NO: 5317); XM_014116470.1, RARA, C_7, In, 25, driver, PPPHLQALLCLSRQIFRLPLWGQRL, (SEQ ID NO: 5318); NM_001110767.1, MYH9, C_7, In, 26, driver, essential, high expression, PHLCHHRHRLQEYDARPRGPVHFVHG, (SEQ ID NO: 5319); XM_014114672.1, HCFC1R1, C_7, Del, 26, high expression, PWYSPHLCPHSGARALSCFSGATLGT, (SEQ ID NO: 5320); XM_005639078.2, SCARB2, C_7, Del, 26, high expression, PCLCMPSSISSMSPIQRRSSEGRSLG, (SEQ ID NO: 5321); XM_005629518.1, EZH2, C_7, In, 26, driver, essential, PKEEEEETPVVGGTLQKDTAEKGRLL, (SEQ ID NO: 5322); XM_014110313.1, TRIO, C_7, Del, 26, essential, PRRTAARGAARARCPWQSPGLGPPRL, (SEQ ID NO: 5323); XM_014111220.1, ELK4, C_7, Del, 26, driver, PSPAPGPPAATTIYTPACILPSLSTL, (SEQ ID NO: 5324); XM_005638000.2, PLAG1, C_7, In, 26, driver, PDTGSSGSCKHPRSQLPALSVSSIHQ, (SEQ ID NO: 5325); NM_001130437.2, PABPN1, C_7, In, 27, essential, high expression, PGSSGPWAWLGSPRQPGGGGGAGTGRG, (SEQ ID NO: 5326); NM_001194984.1, RAN, C_7, In, 27, essential, high expression, PRGGHGSSSGSTVRARSRGCSDDRAPG, (SEQ ID NO: 5327); XM_005617981.2, PRDM2, C_7, In, 27, high expression, PSLSSRVDCCHATASTPSHRPSSGPLF, (SEQ ID NO: 5328); XM_547482.5, DUSP27, C_7, Del, 27, high expression, PRLEVPRDGRPLARVRRSQKTKRTWRG, (SEQ ID NO: 5329); XM_005618371.1, MAN2A2, C_7, In, 27, high expression, RAACHRRPFLLGGGGILRAYSPGGPAI, (SEQ ID NO: 5330); XM_535500.5, MYO1E, C_7, Del, 27, high expression, PRLQDTIRMESSKTSLCHILMLLETRG, (SEQ ID NO: 5331); XM_843145.5, PDIA4, C_7, Del, 27, high expression, PRFPWLRWMPLRKQTWPRGSRSLAIPR, (SEQ ID NO: 5332); XM_532844.5, SLC25A4, C_7, Del, 27, high expression, PSNPHRSSMPANRSVPRSSTKGSLIVW, (SEQ ID NO: 5333); XM_005630260.2, EIF2B4, C_7, In, 27, essential, PGTGGSGDHGAGDDSLQFCTGCSTSQE, (SEQ ID NO: 5334); XM_014116196.1, HGS, C_7, In, 27, essential, PTATPCGPAAACAGPSCTGQRGPAHLV, (SEQ ID NO: 5335); XM_848382.3, EBNA1BP2, C_7, Del, 28, essential, high expression, PRCRVRTRNLRILWSRTESCRMRFPEAS, (SEQ ID NO: 5336); XM_005635260.2, OSBPL2, C_7, Del, 28, high expression, PERMGFRNTGRHCPPRCLPEVISVCGVY, (SEQ ID NO: 5337); XM_534280.5, PIK3CB, C_7, In, 28, high expression, PGGLRASRFQLSRSVRARICCGLPATDE, (SEQ ID NO: 5338); XM_014113910.1, RERE, C_7, Del, 28, high expression, PLRSLWTLPHSCSNLSKRKTMGSVGSIA, (SEQ ID NO: 5339); XM_005642194.1, AEBP1, C_7, Del, 28, high expression, PLQWTITSGLPDPRNPTRTWRQMKRRRS, (SEQ ID NO: 5340); XM_014115267.1, ENAH, C_7, Del, 28, high expression, PHLPLRLPLPPSLHLDFLRHPRPKTIAL, (SEQ ID NO: 5341); XM_014110201.1, PHLDB2, C_7, Del, 28, high expression, PGPRWAAPGGRGRGTGSRPPAARSTTAT, (SEQ ID NO: 5342); XM_844754.4, PLEKHO1, C_7, Del, 28, high expression, PAPTRCGPRTPPPSFRPTRPSCPGSRTW, (SEQ ID NO: 5343); XM_014108793.1, SORBS1, C_7, Del, 28, high expression, PLGLLSLKRTALHGSAPPPPVPPRTPST, (SEQ ID NO: 5344); XM_005620646.2, TAF1C, C_7, Del, 28, essential, PSLSLPVATPSPHSPCWSPGVSGGCRSV, (SEQ ID NO: 5345); XM_545182.4, ICE1, C_7, In, 28, essential, PSGILTTPEHFTFFARTRNLLWRVYRFQ, (SEQ ID NO: 5346); XM_533193.5, CKAP5, C_7, In, 29, essential, high expression, RRLFHGHAAQQTLSAPGVPGAAPALRPGF, (SEQ ID NO: 5347); XM_014113682.1, PTPRK, C_7, In, 29, driver, high expression, QCWPYRGALQCWCWTDWLLHRDRHHAGHG, (SEQ ID NO: 5348); XM_005639643.2, CCDC80, C_7, In, 29, high expression, PKEEGCAGAQGGAPRAPRAAAHQRGPREA, (SEQ ID NO: 5350); XM_014106771.1, XIRP1, C_7, Del, 29, high expression, PWTPVWGWGISEARGAPPTSHRPWKSQCL, (SEQ ID NO: 5351); XM_005619965.2, ELPS, C_7, In, 29, essential, RGACPCSRPATHGASWPRPHGSIEQPGPD, (SEQ ID NO: 5352); XM_014116196.1, HGS, C_7, Del, 29, essential, PNSHPLWPSRRLRRALLHRAARPSSSRLT, (SEQ ID NO: 5353); XM_003639850.3, VPS16, C_7, In, 29, essential, PEADGLVQPSSQQREGCRGGMGEASNGGG, (SEQ ID NO: 5354); XM_005632684.2, CRTC1, C_7, In, 29, driver, PREPGPAANGDRHYLALLHLGQRVWGLVL, (SEQ ID NO: 5355); XM_543674.5, SMARCD1, C_7, Del, 30, driver, essential, high expression, PGGAGRSGSGSFAACGAAPGCRRPGRASLL, (SEQ ID NO: 5356); XM_014116626.1, HSF2, C_7, In, 30, high expression, PRRAVGGAGTSKRSCFHWCSLKWPEFSGFG, (SEQ ID NO: 5357); XM_014115490.1, KCTD1, C_7, Del, 30, high expression, PPIPPTTTAPSRPRWGTPISSPKTARSGPT, (SEQ ID NO: 5358); XM_546571.4, KIF1C, C_7, In, 30, high expression, PSPSVPFTSPRTQWGAGAIVLPQCRAPDWA, (SEQ ID NO: 5359); XM_005618546.2, LDB2, C_7, Del, 30, high expression, PPWGTAALGAVNPPPLRRPNQKAPRPRLPS, (SEQ ID NO: 5360); XM_014109759.1, NRIP1, C_7, In, 30, high expression, LPRARGLRGLQAGAGASERLCGRRRRQGPH, (SEQ ID NO: 5361); XM_843647.4, PDLIM3, C_7, Del, 30, high expression, PSQMCTGCSMTIGMNLLSLASRALSECSRN, (SEQ ID NO: 5362); XM_846534.4, PRCC, C_7, In, 30, driver, essential, PGPSQAKEEDGAREDRGAGAASGGFRFRGR, (SEQ ID NO: 5363); XM_014121534.1, VHL, C_7, Del, 30, driver, essential, PPPPGPPFLPGRSERLLEPPEFVTDAVGRY, (SEQ ID NO: 5364); XM_844090.4, INO80, C_7, Del, 30, essential, PTRAAVICWSLWMTQPPQPPSLGPPTLLLP, (SEQ ID NO: 5366); XM_849952.3, INTS5, C_7, In, 30, essential, PSPPSTPTLPCPCWGTWSRRCGAGSTAGAV, (SEQ ID NO: 5367); XM_014117262.1, TUBGCP6, C_7, Del, 30, essential, PGHCGGRGTTSCTASTSAGTCPTAATTAQT, (SEQ ID NO: 5368); XM_014117254.1, NCAPH2, C_7, In, 30, essential, PCGGGSRTETQEEGCHQAAGLSPVVPGCLR, (SEQ ID NO: 5369); XM_014114743.1, AXIN1, C_7, Del, 30, driver, PATQTWAAWDCGTHTRRTPRASWMSTCSGS, (SEQ ID NO: 5370); XM_014117336.1, MKL1, C_7, Del, 30, driver, PRYAASRLPVAAPARVLLGPVGWHVRTAPH, (SEQ ID NO: 5371); XM_014113911.1, RERE, C_7, In, 31, high expression, HCGPPPFCFFPPGPEPPGEGETGPGGPPAAA, (SEQ ID NO: 5372); XM_538150.5, IL13RA1, C_7, Del, 31, high expression, PILSVSSSKMVTCMCNGRIHRIFIADAYLTK, (SEQ ID NO: 5373); XM_005637886.1, NSMCE4A, C_7, In, 31, essential, PWKVDPARAVHPPPPRPAGDPGTRAWPRCCR, (SEQ ID NO: 5374); XM_542234.5, MAML2, C_7, In, 31, driver, PDGLPSSRRHSPTAPARLSPSPPAAPAQQWQ, (SEQ ID NO: 5375); XM_846083.4, SUFU, C_7, In, 31, driver, PNMASRANAGLGQIRVPVREHLLQRGPRVLA, (SEQ ID NO: 5376); XM_005631964.2, ZEB2, C_7, In, 32, essential, high expression, LERLFITQVARKAYGLHNFTIHSRTPQQCNEL, (SEQ ID NO: 5377); XM_543139.5, PDS5B, C_7, Del, 32, high expression, PAKPNTPFIVSMRYFLVKRPSLHRYLSLCIRA, (SEQ ID NO: 5378); XM_014112907.1, SEC24C, C_7, Del, 32, high expression, PSIFNIWIILANVSMLMTVLSYPWALMNSWPL, (SEQ ID NO: 5379); XM_014115482.1, DTNA, C_7, Del, 32, high expression, PSVWSGCPFCIDWQMWKMSSIRLNVLTATVRV, (SEQ ID NO: 5380); NM_001003016.2, MCL1, C_7, Del, 33, essential, high expression, PRGCCCSRPPAARRRLKRWKARPPTPSCRPKRS, (SEQ ID NO: 5382); XM_005639723.2, BCL6, C_7, In, 33, driver, high expression, QVHILRLAVPTARGDVPPHRWAHIPRGDGGDPV, (SEQ ID NO: 5383); XM_005640837.2, MIA3, C_7, Del, 33, high expression, PRKAGVARWKGCSHTLKIISHKVTPKIISHKTT, (SEQ ID NO: 5384); XM_843925.4, DAP, C_7, In, 33, high expression, QADRVHLRSDRPGRQRLPTSSRPGGSPEAPRLH, (SEQ ID NO: 5385); XM_005617069.1, PARD3, C_7, Del, 33, high expression, PAKASRVPRRTPGTPATKDPGTATWAGTASMPG, (SEQ ID NO: 5386); XM_005616406.2, ZC3H4, C_7, Del, 33, essential, PRCLCQCMSHCPRSSCSSSRTCTPVRSPPCLRS, (SEQ ID NO: 5387); XM_014114365.1, SETD1A, C_7, Del, 33, essential, PRRQPMAYPMLCTHKGKKAGGHTPGKPTTCLCL, (SEQ ID NO: 5388); XM_005619086.2, ZMIZ1, C_7, In, 33, essential, PALPPSGHAQQHGRPRETPQSPHAGNYATRWQF, (SEQ ID NO: 5389); XM_014121720.1, ARHGAP21, C_7, Del, 34, high expression, PARAPGQALPQETQQPRSRRRRLRSRPRVVRRVA, (SEQ ID NO: 5390); XM_014121720.1, ARHGAP21, C_7, In, 34, high expression, PHAPLARPCPRKRSSPEAGGAASDRARASSAASL, (SEQ ID NO: 5391); XM_014121712.1, ARHGAP21, C_7, Del, 34, high expression, PRTRPWPGLAPGNAAAPKPAAPPQIAPARRPPRR, (SEQ ID NO: 5392); XM_537251.5, ASH1L, C_7, Del, 34, high expression, PHFLLLPICMLAIYFSIQPNTIRKSISYFARRPF, (SEQ ID NO: 5393); XM_003638834.3, RYR1, C_7, Del, 34, high expression, PHPAQLSPPTTSTPCWVTSCESSSTTWALTRPHG, (SEQ ID NO: 5394); XM_014112788.1, LSM11, C_7, In, 34, essential, RVGGGRVSLRGSGSSAGRLTPPAPWRSPPDLCRT, (SEQ ID NO: 5395); XM_005637152.2, ETV6, C_7, Del, 34, driver, PPLNCCTVLDHLSQPITGLLPTPSSGPSGPPWTT, (SEQ ID NO: 5396); XM_546606.6, PER1, C_7, Del, 34, driver, PVLRGVPRRGALGVVLGPRLPVRRLLSQRPDWWR, (SEQ ID NO: 5397); XM_014112966.1, TULP4, C_7, In, 35, high expression, PPLLLPLHRLHPYHQAPNSRSQQHERLCQLPLRGQ, (SEQ ID NO: 5399); XM_005634029.2, MBNL2, C_7, Del, 35, high expression, PRAARWRTEESLPASIPSRAVVQERTASIFILQHI, (SEQ ID NO: 5401); XM_014107051.1, ZNF335, C_7, Del, 35, essential, PHCSVLTPLVGPPSSTSKVLRSRQQWPRRRPWICC, (SEQ ID NO: 5402); XM_014114365.1, SETD1A, C_7, In, 35, essential, PGGSLWPTLCFVHTRARRPGGILPGSLPPAFACSS, (SEQ ID NO: 5403); XM_005619085.2, ZMIZ1, C_7, In, 35, essential, PALPPSGHAQQHGRPRETPQSPHAGNSPSRTHKSR, (SEQ ID NO: 5404); XM_005636883.1, KMT2D, C_7, Del, 35, driver, PSQGHWAVRPLLLPPPFSLAAPQPPPACLPLRTGS, (SEQ ID NO: 5405); XM_845833.4, LDB1, C_7, In, 36, essential, high expression, LPSRHHARSGCGPNSHVPAYIPGAWDWEAHTIWQPN, (SEQ ID NO: 5406); XM_005628902.2, PABPC4, C_7, In, 36, high expression, PGAEADAGRTFVPTHPNHAFKPGWKNHRHAAGDRQL, (SEQ ID NO: 5407); XM_003433467.3, TARBP2, C_7, Del, 36, essential, PWRRSPPSPLSSLSAILLAPCRSWWCRRAGGCLSTR, (SEQ ID NO: 5408); XM_003638871.2, PTCH1, C_7, In, 36, driver, PGLRAPSSWATRPAAPEGTPQRRLAAAPLQTAQRRF, (SEQ ID NO: 5409); XM_005625532.2, STATE, C_7, In, 37, driver, high expression, PGSEDSDQVPGWGSILTGPAVPGGPRQASAGQGRHGD, (SEQ ID NO: 5410); XM_544279.5, KLF6, C_7, In, 37, driver, high expression, RGCSEQPRLCLQPGDQQPELRREQRVLRQLRGTFSHH, (SEQ ID NO: 5411); XM_545954.4, KLF3, C_7, In, 37, high expression, PASTVARESSISHSTTREETFACGVPGHPAETKDTQM, (SEQ ID NO: 5412); XM_846233.4, PPFIA1, C_7, Del, 37, high expression, PHTPHLCSRAPPLRTVGAPPHEGARTAQPGRWTDWGS, (SEQ ID NO: 5413); XM_005620011.2, VAMP2, C_7, Del, 37, high expression, PLPPRLGREAPRRPLQTSPVTGDCSRPRPRWMRWWTS, (SEQ ID NO: 5414); XM_005639078.2, SCARB2, C_7, In, 37, high expression, PACVCPVLFLQCHQSRGDPPRGDPSVRRSGAIYLQGT, (SEQ ID NO: 5415); XM_014106771.1, XIRP1, C_7, In, 37, high expression, PGPQYGAGASQRPGGHLPPPTDHGKASASSWGEKARR, (SEQ ID NO: 5416); XM_014114365.1, SETD1A, C_7, In, 37, essential, PAASTPSSSTPSSSSIPGFPAPCLSSPPAGIPLPTAP, (SEQ ID NO: 5418); XM_014112912.1, ARID1B, C_7, In, 37, driver, PATPHQKGDHLSSWLSRGVAASLETKAEDYLKRYRYS, (SEQ ID NO: 5419); XM_533193.5, CKAP5, C_7, Del, 38, essential, high expression, PSPLPRTCCTANSLSSGSPGSSTSTQTWILTRLTLQEA, (SEQ ID NO: 5420); XM_014106445.1, COL4A2, C_7, Del, 38, high expression, PDWACLAPKASVVSPEMPDYLDPQAFPAPPDPQALQDK, (SEQ ID NO: 5421); XM_014108804.1, SORBS1, C_7, Del, 38, high expression, PRQARSQTSPASPVRTRPPLGGSPRFPNACLPDTPWRG, (SEQ ID NO: 5422); XM_846459.4, SMG1, C_7, Del, 38, essential, PQISLLKSSGHLVPHLMNGGESHSLMPGLLQLCLWSDT, (SEQ ID NO: 5423); XM_014108204.1, CABIN1, C_7, In, 38, essential, PATQAGGHPQHGLSGPRGRRAGRRGGGHGLPAPGAPAQ, (SEQ ID NO: 5424); XM_014116841.1, DPH1, C_7, In, 38, essential, PDPQAAPAGEGGVHVPLSSRGLRRLQLRRRKGSAAGSV, (SEQ ID NO: 5425); XM_014116841.1, DPH1, C_7, Del, 38, essential, PRPPGGPSWRRRSPRATLQSRLATVATAETKRERRGLS, (SEQ ID NO: 5426); XM_005629808.2, KAT6A, C_7, Del, 38, driver, PSSHSSHRRPPHRHHSPSPSSHSLRPHRSSSPRCHSAV, (SEQ ID NO: 5427); XM_542234.5, MAML2, C_7, In, 38, driver, PTGPCCPPDGLPSSRRHSPTAPARLSPSPPAAPAQQWQ, (SEQ ID NO: 5428); XM_861806.4, SF1, C_7, Del, 39, essential, high expression, PFPGPKGASSRGHRLLPGLGRGCWCPGSRRPRPWARWEP, (SEQ ID NO: 5429); XM_005639159.2, AFF1, C_7, Del, 39, driver, high expression, PCHLYIPTSKLFPGHKEAARFTAVTVIVKAIAQPNLPKT, (SEQ ID NO: 5430); XM_014111003.1, MAP2, C_7, In, 39, high expression, RGRAGSRPVHVGASGCRVWPREGRRPGQRDGDQGEGGQA, (SEQ ID NO: 5431); XM_853562.3, R3HDM2, C_7, Del, 39, high expression, PRTHPWSSGVTVNITAWTSVGRNLETYTILTVAPRPTLK, (SEQ ID NO: 5432); XM_846492.4, XIRP1, C_7, Del, 39, high expression, PHHRRVPIPQERGPQLVPSKGPLRTRRFCREASKSSRAC, (SEQ ID NO: 5433); XM_014121783.1, PTPN23, C_7, Del, 39, essential, PRPQAPLLWSWPLVLLSTLPLPTLQSWDLCPGRPHSTVW, (SEQ ID NO: 5434); XM_014111220.1, ELK4, C_7, In, 39, driver, PARRQGLQPQRLYTLRLVFFLHSQLFELLQQEAFQTHKD, (SEQ ID NO: 5435); XM_014106445.1, COL4A2, C_7, Del, 40, high expression, PESSGFLGSQEVGVTRELQGEQAFTARWAPQGTLVTVETS, (SEQ ID NO: 5436); XM_014106445.1, COL4A2, C_7, In, 40, high expression, RTGPAWPQRRAWFPRRCRITWTPRLSRPPRTPRHSRTNRL, (SEQ ID NO: 5437); XM_005631375.2, PPFIA1, C_7, In, 40, high expression, PTPHICAHGPLPSAQWALHPTKGPAQPSQGGGQTGGHDST, (SEQ ID NO: 5438); XM_845916.4, COL3A1, C_7, In, 40, high expression, RPRGPGGEERRPRRDGPRWSLRRSWSCRLPRPPWSPRSTR, (SEQ ID NO: 5439); XM_014112944.1, LDB3, C_7, Del, 40, high expression, PGPKCPPWPGASCSGRSASRPAAGPRSAATATTSSGAPFW, (SEQ ID NO: 5440); XM_014122040.1, BRD4, C_7, Del, 40, driver, essential, PKCYCRRTKSHLPRPSPLCRCSSTCSSCRRCSPPHHYSLP, (SEQ ID NO: 5442); XM_543631.5, CALC0001, C_7, Del, 41, high expression, PRGGSALFVRSASQPRVIRTPWRTTWMDTSFSAPRTPLPLS, (SEQ ID NO: 5443); XM_005634029.2, MBNL2, C_7, In, 41, high expression, QELPGGERKSHCLLRFPQGPLFKRELQVSSSSNTFKNSTRN, (SEQ ID NO: 5444); XM_014108204.1, CABIN1, C_7, Del, 41, essential, PGHTGRRPPPAWSLWAQREKSWQEGRRAWASRPRSPCTVSR, (SEQ ID NO: 5445); XM_014107093.1, PTPRT, C_7, In, 41, driver, PRAAGRRGHIPVDQAKCQLNHRGWPHHPEGGGISHHYRHLG, (SEQ ID NO: 5446); XM_545531.5, WIPF1, C_7, In, 42, high expression, PALPWKPRCSFCRRLPPPDILKLLIILLQQASPAPHAQQGLG, (SEQ ID NO: 5447); XM_014113658.1, TOP3A, C_7, Del, 42, essential, PELPSQLASPLTTCKLASPRTERTAPPGCCPCPPPPMGKVIL, (SEQ ID NO: 5448); XM_005630709.2, BCL9, C_7, In, 42, driver, PCLGVCCCFACSPQVSITSCPVTWMDLLSKTSPSESRDPSKP, (SEQ ID NO: 5449); XM_861806.4, SF1, C_7, Del, 43, essential, high expression, PASPPFPGPKGASSRGHRLLPGLGRGCWCPGSRRPRPWARWEP, (SEQ ID NO: 5451); NM_001287169.1, SLC7A8, C_7, Del, 43, high expression, PSLAFDSWLPSAYCSSHGSTVPVCGGPPGFKTFSQLGSSWPWP, (SEQ ID NO: 5453); XM_014117262.1, TUBGCP6, C_7, Del, 43, essential, PATERSPTSRRPAGTPSTGSAGSAEGSCRRSAGACCRSRSPCW, (SEQ ID NO: 5454); XM_005618689.2, GAK, C_7, Del, 43, essential, PLPRAAAPGSRRVGPRPRAPRGPPRPSRPPRPVRSQGLTMLPT, (SEQ ID NO: 5456); XM_014114048.1, ZFHX3, C_7, Del, 43, driver, PNPSSSSSSHRCSRRRPQLSHHPHHSSHRNSSNSNARTKTARR, (SEQ ID NO: 5457); XM_546571.4, KIF1C, C_7, Del, 44, high expression, PQPQCPLHIPPHTMGSWSHRSPPVPSPRLGLRRPWRGCRRQRRS, (SEQ ID NO: 5458); XM_544279.5, KLF6, C_7, Del, 45, driver, high expression, PGRCAEGLRGSPVTRAAGTPPRMAGGGCIAATLTAAGKFTPKAPT, (SEQ ID NO: 5459); XM_846233.4, PPFIA1, C_7, In, 45, high expression, PFLPKIAPLRQAAQRSTAHSQPRRHQGCQKLHRLTGWPREQPQQQ, (SEQ ID NO: 5461); XM_535696.4, ENPEP, C_7, Del, 45, high expression, PRRALRPRPRRPGTRGSVPPAKMTAGTGRTSGCRTSSARCTTTWR, (SEQ ID NO: 5462); XM_005639813.1, ST6GAL1, C_7, Del, 45, high expression, PGPARPSAVPGAQSRPSPRPPSRCGTRTALPKTSSPGYRRSGEII, (SEQ ID NO: 5463); XM_005620586.1, ZCCHC14, C_7, In, 45, essential, PDDGRSLSPSAHPDAEFGAQVGKRGCGHEAPPAFRALAAVSRRKE, (SEQ ID NO: 5464); XM_014108440.1, PCBP2, C_7, In, 46, essential, high expression, PEGRDHPVPAQAVQFSSHLCRWSGQVQHRQRQCELSPHHPVHVPQP, (SEQ ID NO: 5465); XM_005635260.2, OSBPL2, C_7, In, 46, high expression, HQCILFRRPQSGLPVSRLHLPEAEVLGQERGGRAPGDHHAGAAQTQ, (SEQ ID NO: 5466); XM_005616171.2, TNNT1, C_7, Del, 46, high expression, PRSQKGNVWTSMTSTASAWRKICWSCRRSSTCILNSGRKRKRSSLR, (SEQ ID NO: 5467); XM_533117.5, FDXR, C_7, In, 46, essential, PDLCGGQWTRWLLYRPTLAKAPPPGPCGHLREAACAFRPGALWRGA, (SEQ ID NO: 5468); XM_005616269.2, POLD1, C_7, In, 46, essential, PGSPEGAPHLPAVGDRSLCGPSTAPARSTHAIPRLRAYPASLWSHR, (SEQ ID NO: 5469); XM_846145.3, EGR1, C_7, In, 47, high expression, PRTPVRLPGRVLRPSLLPLRRAHAPHPHPHRPEALPVSHLHAQLQPQ, (SEQ ID NO: 5470); XM_014111713.1, ATP2B3, C_7, In, 47, driver, PAPVPQPEQQRHRQRHLPEHARHQVSYLVSAFFQTGEPPPQRGDVPL, (SEQ ID NO: 5472); XM_005639813.1, ST6GAL1, C_7, In, 48, high expression, QGQPGPRQSPGPSQGQVRGLLPGVEQGQLFQKPHPQATEDLEKLSEHE, (SEQ ID NO: 5474); XM_014109523.1, PML, C_7, In, 48, driver, PEPASCQPAGPEHLLQRPVGGGRLRSGTRPLHPSHWPQLCKEGMPAEL, (SEQ ID NO: 5475); XM_014108447.1, PCBP2, C_7, In, 49, essential, high expression, PEGRDHPVPAQAVQFSSHLCRWSVDQAAPVGNATVSFSHDAWQHRIQWH, (SEQ ID NO: 5476); XM_014107801.1, CCDC92, C_7, Del, 49, high expression, PSPPTAAQVSGTAQPASSSTRLMWGWPIASTMLPHHTPSLSWRRWQSTR, (SEQ ID NO: 5477); XM_846233.4, PPFIA1, C_7, In, 49, high expression, PTPHICAHGPLPSAQWALHPTKGPAQPSQGGGQTGGHDSIAGFPGRGTR, (SEQ ID NO: 5478); NM_001003295.2, HSPB1, C_7, Del, 49, high expression, PRSRAPPRPPRPPTAARSAGSSAAACRRSGRRPTAGACPWTSTTSPPRS, (SEQ ID NO: 5479); XM_541508.4, PLEKHA4, C_7, In, 49, high expression, PGLNFSPKPGDRYSVDQAVWAGPAPEEAAGGDRPEAGGKGAARSSLGVN, (SEQ ID NO: 5481); XM_844754.4, PLEKHO1, C_7, In, 49, high expression, HPPSAPPGPAVPDPGPGSQETGKDSGAAGRGSGPGRREAEGQGPPLVSP, (SEQ ID NO: 5482); XM_014118655.1, ZC3H3, C_7, Del, 49, essential, PGATIGLRRGRASPLVGSGNPQSQEESRGAAVQRTPFWSARRSPASPGW, (SEQ ID NO: 5483); XM_005636883.1, KMT2D, C_7, In, 49, driver, PARGTGQSAPCCCPHHFHWQPHNPRRLVYLCGRVPEAASGHSARPRLAR, (SEQ ID NO: 5484); XM_014118179.1, PRDM1, C_7, Del, 49, driver, PGGRTATAPTFHPSPPRGTPTTSEKTGAPRCPSTPGPCTRVGSLCPRTF, (SEQ ID NO: 5485); XM_014121877.1, MAP4, C_7, Del, 50, high expression, PLGRSQPSPRQRPKLAPPLQPVASVATPPWQGVVTKGRPRPWTARSRRQI, (SEQ ID NO: 5488); XM_014120931.1, MUC2, C_7, Del, 50, high expression, PSTTTTPACSTPASCPAQAWSVPACRPTRPCAPTRASVSTGGTTPTGFAR, (SEQ ID NO: 5489); XM_856636.4, BRPF1, C_7, Del, 50, essential, PSSPSWAPYVSASGVGAPGPVRAQTATVINPQKTPQWTYQPMASAAEISQ, (SEQ ID NO: 5491); XM_005631240.2, SPI1, C_7, In, 50, essential, PAAAALPPHGAGADARARHPHGSEPRGHRPPGLLSTPDVPPIPLSVPGPA, (SEQ ID NO: 5492); XM_005619043.2, ZSWIM8, C_7, In, 50, essential, LLCCMPGHNLVLFLSVSPSGFCSLPACPSLRVPVSAAEGPAAEVCPVPHQ, (SEQ ID NO: 5493); XM_005632684.2, CRTC1, C_7, Del, 50, driver, PQRTRASRQWGSTLPRTPPPWAACLGTRTMSSRWRPGRPMLCPTSWSSST, (SEQ ID NO: 5494); XM_014119360.1, CCDC9, C_7, In, 51, high expression, PTWRPGRHGPGSPQLGGQLRGAASRRSWGPWPERPGPRVPSSFWSWRYLSG, (SEQ ID NO: 5496); XM_014121887.1, MAP4, C_7, Del, 51, high expression, PLGRSQPSPRQRPKLAPPLQPVASVATPPWQGVVTKGRPRPWTARSRRQAS, (SEQ ID NO: 5497); XM_005626251.1, ANTXR1, C_7, Del, 51, high expression, PHPLHPIALPHPPVLPPLRFHPHRPPFPLPLTPHLPAGRHPPPDLLPGLLS, (SEQ ID NO: 5498); XM_005626251.1, ANTXR1, C_7, In, 51, high expression, PTPCTPLPSPTPQCSHPSDSIPTVHPSPSPSRPTSQQGATPLPTSSQAFCL, (SEQ ID NO: 5499); XM_014115267.1, ENAH, C_7, In, 51, high expression, PPCIWIFCGIRVRRQSPFDWTCGCHRRSETEESVPDGGCLFPRRRCEPSLI, (SEQ ID NO: 5500); XM_005629454.1, TMEM192, C_7, Del, 51, high expression, PTAILTGLLTGVPWISPRVLKMTPFWIPTISHIIHYMLILDPDSILFLRSS, (SEQ ID NO: 5501); XM_861377.4, COX11, C_7, In, 51, essential, PTPGRYGRALASPVETRCLLWLALEPPWLRGQGRGEGRAPSQAGNEWGRRC, (SEQ ID NO: 5502); XM_014108204.1, CABIN1, C_7, Del, 51, essential, PARPSRASCPACQSWSSPRPPPSSPLRSPSRRPPQLCSHPKAASRRRPSRS, (SEQ ID NO: 5503); XM_860093.4, EWSR1, C_7, Del, 52, driver, essential, high expression, PKLDPTARLQVNIANRAAATGSRVHSDRTTPVAWVFMGRSLEDFPDQERTGA, (SEQ ID NO: 5504); XM_845091.3, IGFBP4, C_7, Del, 52, high expression, PWAARSWCGSRAAAVAPLAPWARGCPAGCIPLAAARACAATRPGGWRSPCTR, (SEQ ID NO: 5505); XM_547871.5, DCAFS, C_7, Del, 52, high expression, PQASPPIPTLEKTTMITPRSKWMTSPLPLPPPLSAALPRWRFSPTGHLQLPT, (SEQ ID NO: 5506); XM_014116626.1, HSF2, C_7, Del, 52, high expression, PQESSRGRRYQQTQLLPLVLTQMARVFWFWMNKDLQKKFFLNISSTITWQAL, (SEQ ID NO: 5507); XM_534998.5, PPRC1, C_7, In, 52, essential, PTQEVAKIQLQFLWTFPKMLFLFFLLIFFLFFLIFVIQFSKPVPLPIPPTEK, (SEQ ID NO: 5508); XM_005629808.2, KAT6A, C_7, In, 52, driver, PAATAATAARPTATTAPAPAATASAPTAAAAPAVTVQYEQQFHSSAHDHGDP, (SEQ ID NO: 5509); XM_536455.5, CYFIP2, C_7, In, 53, high expression, PLQDHLQTPGLPGHRSGHGGTAEDCQELAPRNHSPVRENTDRGDAQDMPLTPT, (SEQ ID NO: 5510); XM_014118288.1, LENG8, C_7, In, 53, essential, PSAAASSPAPSALQPPAWGSCSEQWPPAWNSPSHTAQPGRACLRPGLWATCLL, (SEQ ID NO: 5511); XM_541488.5, PNKP, C_7, In, 53, essential, RGSPPHLPALGRASPCPGPGTLDPGYRPEVLQKPSGAGRKPQDQDRGSETAGN, (SEQ ID NO: 5513); XM_005637736.2, TCF7L2, C_7, In, 53, driver, LRLHCQHSSLQGHEKEPLLTQGWGALVFGVEQSARRAAPSPCSPSHAAHHVQQ, (SEQ ID NO: 5514); XM_014116626.1, HSF2, C_7, Del, 54, high expression, PPPQESSRGRRYQQTQLLPLVLTQMARVFWFWMNKDLQKKFFLNISSTITWQAL, (SEQ ID NO: 5515); XM_852921.4, BRD3, C_7, Del, 54, driver, PLSPRRLSLLPPLCQPLLQTSHRSLFPRPPPHLLPRRPSSLWSLPHRLSSRKRV, (SEQ ID NO: 5516); XM_014113507.1, ARRB2, C_7, In, 55, high expression, RAQPTPAPHSPAGPAAEEAGPACPPIFLHNTPEPALLCHPAAGPRGHGEGLWSRL, (SEQ ID NO: 5517); XM_533751.4, LMCD1, C_7, In, 55, high expression, PWSHPETGSAVHGAHPKREAASDRHRGCLLPPTPAHAPAPHLRPGPFSLPWTSGE, (SEQ ID NO: 5518); XM_005631208.2, TNKS1BP1, C_7, In, 55, high expression, PVPGRATGTERPEQLAWWRGWPGGWRAAGEAAGGGPEPRRCWPGRQGGGPPRLGG, (SEQ ID NO: 5519); XM_014116470.1, RARA, C_7, Del, 55, driver, PSPASTSLALSVKTNLQATTMGSAPVRAARASSAAASRRTWCTRVTGTRTVSSIR, (SEQ ID NO: 5521); XM_005639752.2, SOX2, C_7, In, 55, driver, RGYLLLPLQGALPGRGPPGHDQHVPPRRRGAGARRPQQTAHVPALPERPGARHGH, (SEQ ID NO: 5522); NM_001130437.2, PABPN1, C_7, Del, 56, essential, high expression, PRELRALGLAREPPAARRRRRSRDWSRVTRGTAPLRTRSWKRSKLESGRWRKKLRS, (SEQ ID NO: 5523); XM_014108796.1, SORBS1, C_7, In, 56, high expression, QHPQPAETLTTAACPCLPGSQIHSQRCAAPQAVASRVHVLPCFLPTLAPLCGHQDS, (SEQ ID NO: 5524); XM_014117254.1, NCAPH2, C_7, Del, 56, essential, PVWRRFQDRNARGRVPPSCRTFTSGTWLPTLTMPTAGGPGGRARLLQTWRSCTGSM, (SEQ ID NO: 5525); XM_005632683.2, CRTC1, C_7, Del, 56, driver, PQRTRASRQWGSTLPRRRLCSSTVLVPAPRLTSLPPHLSPIKASPLGAPRNWSSST, (SEQ ID NO: 5526); XM_005630809.2, POGZ, C_7, In, 57, high expression, PAPLGKKIHPKSLEPEGGFLSPAGRRALHGSLSAVSPSPRPLPCSHPLLPAPGARSL, (SEQ ID NO: 5528); XM_005630709.2, BCL9, C_7, Del, 57, driver, PLSWGLLLLRLFTSSLHHFLPRHLDGPPLQNLPFRVPGSLQTIKHPSPWPPQPCWAV, (SEQ ID NO: 5529); XM_542234.5, MAML2, C_7, Del, 57, driver, PRRPPKQPPPQPHRPRQTITITTSSTCSAVAIMVAVVGSTGSSRHKLQPLGTRGTQP, (SEQ ID NO: 5530); XM_014108448.1, PCBP2, C_7, In, 59, essential, high expression, PEGRDHPVPAQAVQFSSHLCRWSVDQAAPVGNATVSFSHDAWQHRIQCRFGCICSDYFS, (SEQ ID NO: 5531); XM_537756.4, CPD, C_7, Del, 59, high expression, PGPVVATTAAAVTSTAASPTSSAPASPRPWTRCPRCAPSWTGSAGTSSCFLEICMVAQW, (SEQ ID NO: 5532); XM_014114365.1, SETD1A, C_7, Del, 59, essential, PSSLHPLLLHPLLILHTWLPCPLLILPTSGHTSSHRALMGRLPPSTPHLLRHPPTFMTL, (SEQ ID NO: 5534); XM_005640267.2, XIRP2, C_7, Del, 60, high expression, PGTSCPVSLRLTGRPSPPRQVQPAPRPRPRPRPHPRTRPRPRSPRVARSSWPSSETPPPG, (SEQ ID NO: 5535); XM_014108804.1, SORBS1, C_7, In, 60, high expression, PGRPEARPPQPAPSGRDPLWGEALGSQTPVFPTHHGEAEPDLGAPSGETRSPRGLPEGLS, (SEQ ID NO: 5537); XM_005627265.2, SRPK1, C_7, Del, 61, high expression, PLRWRRASPPASGLPVRAHQPRRPSAPAALAPCSPPGRPASWGRCRARRSATRADPGPPAP, (SEQ ID NO: 5539); NM_001313808.1, TRIB1, C_7, In, 61, high expression, RLALQPAAAARRSGSRRRLRERAGAQPHRRLPAAAAGGARACVPGAVHPHRPRAALQGVSY, (SEQ ID NO: 5540); XM_014115267.1, ENAH, C_7, In, 61, high expression, PTSPSASRSPPPCIWIFCGIRVRRQSPFDWTCGCHRRSETEESVPDGGCLFPRRRCEPSLI, (SEQ ID NO: 5541); XM_014117542.1, BCL11A, C_7, Del, 61, driver, PSLLCNPPLLPPSPRSSPSRASSAARRSNFRATSWCTGAATRARSPTSATCATTRARRPAS, (SEQ ID NO: 5543); XM_845621.5, MLLT6, C_7, In, 61, driver, PTAACRGPGSSLARQQHESHGRGGSSRSSSSSRRTSSPHGPDQPLPQPGGGGWQWPQRRDR, (SEQ ID NO: 5544); XM_014115086.1, NSL1, C_7, In, 62, essential, LPPFRSHSSRPRFKLERWRGLPCRGSLPVRGAWRERRGRTTGRRSPPAPGRTAGCAARRSGP, (SEQ ID NO: 5547); XM_005619085.2, ZMIZ1, C_7, Del, 62, essential, PSSPTLRTCPTTWPPSRNPSVTPCRKLSFQNSQIPMSSSRTWIPLTCRAIVTMTSCLSLRTT, (SEQ ID NO: 5548); XM_014108443.1, PCBP2, C_7, In, 63, essential, high expression, PEGRDHPVPAQAVQFSSHLCRWSGLYHSRTVCHSTARFDQAAPVGNATVSFSFIDAWQHRIQWH, (SEQ ID NO: 5549); XM_005629454.1, TMEM192, C_7, Del, 63, high expression, PQAPREPLLQSPPTAILTGLLTGVPWISPRVLKMTPFWIPTISHIIHYMLILDPDSILFLRSS, (SEQ ID NO: 5550); XM_005637886.1, NSMCE4A, C_7, Del, 63, essential, PLESGPRPGRSPPPPPPRRGPGDPGLAALLQVITPARTCSRVMSTFPQTLFFVCLVFCFFFFA, (SEQ ID NO: 5551); XM_005630809.2, POGZ, C_7, In, 64, high expression, HPLPRGPPAPLGKKIHPKSLEPEGGFLSPAGRRALHGSLSAVSPSPRPLPCSHPLLPAPGARSL, (SEQ ID NO: 5552); XM_005621171.2, INTS1, C_7, In, 64, essential, PHSGPSEKSKPVPASRAPAVPEPKPRLSPQMPEAGLLAVHALAGRPGAVQRGFAGRAACAVPV, (SEQ ID NO: 5553); XM_542234.5, MAML2, C_7, Del, 64, driver, PHRPLLPPRRPPKQPPPQPHRPRQTITITTSSTCSAVAIMVAVVGSTGSSRHKLQPLGTRGTQP, (SEQ ID NO: 5554); XM_005630688.2, NOTCH2, C_7, Del, 65, driver, high expression, PLPQPMLSMHCPSLTCMKCSRWLMVPALCFPQLASCCPTTTLLPQATAVQGAWVGSILSLFRQIG, (SEQ ID NO: 5555); XM_014113186.1, NCAM1, C_7, In, 66, high expression, PGGCAQPERPRRRGGGLQGPSSQQAGPRRRGPPRPAGGPRHRRPGPRARPARRRREEPGRGSRGPH, (SEQ ID NO: 5557); NM_001287169.1, SLC7A8, C_7, In, 66, high expression, RVWPSTPGCHLLIAPHMGQLCQCAVGHPGSRHFHSWEAPGPGPDHHHGSCTDLQRRVFLAGAKEC I, (SEQ ID NO: 5559); NM_001003090.1, COL1A1, C_7, Del, 66, driver, PVLVVALVMPVLLVPLALLDPLVPPVLPAAVSTSASCPSHLRRRLTMAAATTGPMMPTWSVTVTSR, (SEQ ID NO: 5560); XM_005621491.2, GPRC5B, C_7, In, 67, high expression, RPPPPAPRLPTRSPFAKGAAPWSADKGPWSVAGSGVPARGPGPRATLAAQLRQDGGLVFAAVYQP QK, (SEQ ID NO: 5561); XM_014113910.1, RERE, C_7, In, 68, high expression, PALICSAPLCPWGDTHHPSNLLFHLHATCGTRPLSPAAVLCCCFFGRQHTWGDSLPAPHCPDQGRG SG, (SEQ ID NO: 5563); XM_014119360.1, CCDC9, C_7, Del, 69, high expression, PNMEAGQAWAGQPTAGRTVAGSSLEEELGAVAGEAGAEGPLIFLELEIPQWLTANLRSGRSGGGR TSRR, (SEQ ID NO: 5564); XM_544046.5, BAG3, C_7, In, 69, high expression, PRRPRRVPPPPAGLHCHSCPPRRRREPTTASSLCLSPAGDTALPNRGGHRGSSEVPVPSAGCGRGNP AR, (SEQ ID NO: 5565); XM_005626284.2, FLT4, C_7, Del, 70, driver, PTSTRRRLPPPAPTPATVARPSPALPTGCPPLSVSSGTGGHGHPARPSPSAASAGGSRETTCHSARTG ER, (SEQ ID NO: 5567); XM_005636883.1, KMT2D, C_7, Del, 70, driver, PHLRYPACPPCLRCHACPHHLRNLPCPHHPKSPPHPLHLRLPACSHHLRTPRLPRRLKTHLLLRHRR TYS, (SEQ ID NO: 5568); XM_005635182.1, NFATC2, C_7, Del, 70, driver, PRCGRRAPTRRPCLPPRPRPACPATSTRPWSSWGPASRRRAGARPPSPSCSCRPPGPSSWCQLFSPAA SQ, (SEQ ID NO: 5569); XM_005620526.2, UBE4B, C_7, In, 71, essential, high expression, PCPCGWPDGCALHFPQPAWWHGLWNCHRKPALLSTVSPLHCHPPVGVFDFSYPAIPRHLHSVQLA QPPCPC, (SEQ ID NO: 5571); XM_014116817.1, MY018A, C_7, Del, 71, high expression, PGRPARGPGMSWMNAAPPSPRPPAAALAGGWRRGGAQTLTGPPPSLPPPAGPLRPPGVAPGALVR TGPAPP, (SEQ ID NO: 5572); XM_014108446.1, PCBP2, C_7, In, 73, essential, high expression, PEGRDHPVPAQAVQFSSHLCRWSGLYHSRTVCHSTARFDQAAPVGNATVSFSFIDAWQHRIQCRFG CICSDYFS, (SEQ ID NO: 5575); XM_541491.5, SCAF1, C_7, In, 73, essential, HPQEVQAGAQAEWWRGQGGCLIVFGCTARPASPGLPLGLQEAPLPGPQARLACLVVRPPPFAVPLS PPPLAQH, (SEQ ID NO: 5576); XM_014113201.1, MLLT4, C_7, In, 76, driver, high expression, PPAAPGLRRHLSLHLIPAPLGPSQQYCSQSRPAPTPSKKTRLLPAQPLERTKLLPGICRDSCWHPRCL SGPERKTV, (SEQ ID NO: 5580); NM_001313776.1, CKM, C_7, In, 76, high expression, PLLPGRAPGSGETLHRSPQQPDRRVQGEILPSEEHDRAGAAAAHRRSLPVRQTRVPTAAGLRHGPR LARRPRHLAQ, (SEQ ID NO: 5581); XM_014110313.1, TRIO, C_7, In, 76, essential, PGGQPLAEQPGLAAPGKAQAWGHLASELSRGRRITSSNRQGALSPQQPPAEGGLLLELHPRLPRQP ARLLHLPGGQ, (SEQ ID NO: 5582); XM_546778.4, ANKRD11, C_7, Del, 77, essential, high expression, PLRTEMNCWPHPWREPFPLIWAFPWMPQRTSRPLPPSSPQSPATWSHWMRAPSARSSRRNPSSGHI RRPQSRASPAV, (SEQ ID NO: 5584); XM_847792.4, IGFBP5, C_7, In, 77, high expression, QPPGLRAGQRARLRLLHDLRPGRGAVVRRLHGALRPGAALPPPAGRGEAAARPAARPRGLPQREE LPRASQDRERLP, (SEQ ID NO: 5585); XM_005628902.2, PABPC4, C_7, Del, 77, high expression, PRSRSRCWENVCSHSSKPCIQTWLEKSQACCWRSTTLSCCTCWSPLSLSAPRWMKLWRFYRLIMPR KKLPRRIQKPN, (SEQ ID NO: 5586); XM_847992.3, NCOR2, C_7, In, 77, driver, PGGCSSPCSATTTALAARERHPPAAQQQPSRQKQEPCASCRQGGEACVLPGLCTRGPEAAHGGPML DVRPALPCAPS, (SEQ ID NO: 5587); XM_014106920.1, ZHX3, C_7, In, 78, high expression, PFRQTPGLAPDPRLHANQVQGASPRAAQSPGEQFCAEPSSSRRRAGPPEDGNQDDPERDRQLVLRE AEESGCRGHQEG, (SEQ ID NO: 5589); XM_014112944.1, LDB3, C_7, In, 78, high expression, RAPSVPPGQGHRAAGGALPGQQQDPALRPLQQRHQGPLSGSHGPFLAPGGVQLRLLQDLPGGRVL RGGAEQRLLRALL, (SEQ ID NO: 5590); XM_014113564.1, CHD3, C_7, Del, 79, high expression, PSQPAFRCPSAAFSAGWPARARSLTPHRPFPRGPTLHLRDTGRPSAPHPSGPWPPQAPTTARCLPGPS SQPPPTALQCW, (SEQ ID NO: 5591); XM_005630632.1, HIPK1, C_7, In, 79, high expression, PISVVECLLQCEETENRALWLGCFWTEQQRQILYPQQNPPSHTRASQLLSPGSKFQHPCLRPGPPPPS SCSGAYCCNSC, (SEQ ID NO: 5592); XM_014107013.1, PEF1, C_7, In, 79, high expression, PWWRTVWQWGTPRWWLWRGSCPRRALWTTSWWGTLWGAPLWNSRRTIWWCGPGGPLWSATS KFLRCPASWALWTGTSSR, (SEQ ID NO: 5593); XM_534998.5, PPRC1, C_7, Del, 79, essential, PHTRGGEDPVAVPLDVPEDALPLLPPHLLPLLPHLRHPVLEAGPAPHPPDGEVTGGGGTVLIVHMTI TKGRGCCRRSVQ, (SEQ ID NO: 5594); XM_014121783.1, PTPN23, C_7, In, 80, essential, PGPRPPCCGPGPWSCSLPCPCLHSRVGTCAPVVPTARCGKQSLWGGRTTPASCRSALCPSSPVLRTR DGPGGSASHDHCG, (SEQ ID NO: 5596); XM_851907.5, PABPC4, C_7, Del, 81, high expression, PRSRSRCWENVCSHSSKPCIQTWLEKSQACCWRSTTLSCCTCWSPLSLSAPRWMKLWRFYRLIMPR KKLPRRWALLLLLPL, (SEQ ID NO: 5598); XM_005618371.1, MAN2A2, C_7, Del, 82, high expression, PCCVSPKALSSRRWWHTTSIFARWSGYIICQGWRGCLWTCRPWWTSGITSTRSWPCASTQTLTARV PSSRTSMAFRCSPGAI, (SEQ ID NO: 5599); XM_005616269.2, POLD1, C_7, Del, 82, essential, PWIPRRSPSSSSSWRSIIMWAQHSPCPEHPRHPATPCLSCEPLESQMRACPSAATSMALHPTSIPRRPL VLGPSTWVTCSGS, (SEQ ID NO: 5600); XM_534128.5, TSC22D1, C_7, In, 85, high expression, PERCDQWERSSSPPPSPPSHSSRAPPPPRAPPSIPCRRGQSTHSRRATLQPGIQKTLCPWKRRQRCTSC TDFCCIIGRLTCIRND, (SEQ ID NO: 5603); XM_014117262.1, TUBGCP6, C_7, In, 85, essential, PGTAAAAGLLPAQPARRQERALRRLRLLRPERVRDGCALVDLRGGVLVPGRGPRGAAGDGGGPG RRPARRGVGALLLRRPLRGQV, (SEQ ID NO: 5604); XM_005619086.2, ZMIZ1, C_7, Del, 86, essential, PSSPTLRTCPTTWPPSRNPSVTPCRKLCHTLAVLTSPIPPYNKVCTSHTPAASQGLHYITVGLLLLLPS LPGNRHRPLPATIHTVT, (SEQ ID NO: 5608); XM_014122040.1, BRD4, C_7, Del, 87, driver, essential, PSHSRHPSSSTSPRHGLCTCSPCSSLPTSSSPRRPRASSPRTRPRASSRRLHSPPSLSRSSSITLHPGTTSR TPILPVTSEKPPPRL, (SEQ ID NO: 5609); XM_846534.4, PRCC, C_7, Del, 88, driver, essential, PRCWPPPSPRHCCCPRPPGTPGSSPRRPCRSAWEASLPLRASARRRRRELGRGWGCPRPGAPASACP PPWAVPGPPWAFSPQRRGRSP, (SEQ ID NO: 5610); XM_014112562.1, FAM193A, C_7, Del, 91, high expression, PHPPPPMAPSALHPAFAATPTVRGTAVRTACMTHSRTMGMRVPTRTAALSIALAPQPPPTRRRAST VTAATVSSSGMVGLQLHQQVEIMQR, (SEQ ID NO: 5613); XM_005621491.2, GPRC5B, C_7, Del, 91, high expression, PPPAPRPPPPNSFPVREGGGSLERGQRPLECGGQRRPCPGPRPTRHTGCPAAPGWGLGLCGRVPATE VKLGHYSVENGQAPRSPGRGQRER, (SEQ ID NO: 5614); XM_005632690.2, SSBP4, C_7, In, 91, essential, PQPQRPHDGASCSASCGPPWLPAPPPWHHGTLPESSGASKHGPDAESDTSTGHGQRWTPELWRWH AAPTQLPRRPRPAHHEHGPWSARPVG, (SEQ ID NO: 5615); XM_014113201.1, MLLT4, C_7, Del, 93, driver, high expression, PARRARPPTASFSAPHSGPPWPEPTVLLTKQASPHPLQKNQASTSPATRKDQTLTWDLPGQLLAPT MPIGTREKDCLKATRQIYLGALEPLKT, (SEQ ID NO: 5616); XM_005616531.1, CIC, C_7, Del, 93, driver, PTLWGWWNLERVPLPPLRRMPLALQESPGWTVRQRVTTMMPSSPSCLLRSSCPCHPGNAGPSLSVP CQRNETPLQRRMDAAPTSGRRTISGGP, (SEQ ID NO: 5617); XM_005626284.2, FLT4, C_7, In, 93, driver, PHPREGGFLPQHLLPPQ SPGPHLHCLRGAPPSQCPVALAAMDTLQDLHQAQPQPAAAERPHATVPG LARGDHPGCREPHREPGHLDRVCGGEE, (SEQ ID NO: 5618); XM_014107013.1, PEF1, C_7, Del, 95, high expression, PMVEDSMAVGYPQVVAMEGVLPPEGLMDHQLVGDPMGGSPLELQEDYMVVRPRGAPMVSHLQI PTVPSILGLMDRDLLQVSQWTPITVATSPSRS, (SEQ ID NO: 5620); XM_544915.6, PWP2, C_7, In, 97, essential, PGGPAAESPHGLEGRPAAGGGGGEGGRRGGGGGDHRPGQSHAGCRGAAGESQILSAGQVLFQQR GRFQQADRCGLPQEDPSLGHRLCFRNLPPSRAA, (SEQ ID NO: 5622); NM_001204439.1, PPAN, C_7, Del, 98, essential, PRRQRGLASRRKRRTKLSISARQWERSPMRTCSRKQPNADSSPGPQARSHEEKSGGQMEALTTGLQ RPRASPSGTRPSWDGEELSGTAGEAKLDHLRK, (SEQ ID NO: 5623); XM_014115709.1, CCNK, C_7, Del, 99, essential, high expression, PTHPLPQCPLHQPPSPLLPFLPPLLAIPLLPPPTTPTSHPHPHACPPPTQCPLTLLQAWACHRPATPLRL SPLEDSHPCPHPSPHPVCLQLGGWGGQPG, (SEQ ID NO: 5624); NM_001003264.1, SRP72, G_7, In, 8, essential, high expression, GVGACALE, (SEQ ID NO: 5625); XM_005640836.2, MIA3, G_7, In, 8, high expression, GVLPTSHG, (SEQ ID NO: 5626); XM_545627.5, CPS1, G_7, In, 8, high expression, VRLRNLSH, (SEQ ID NO: 5627); XM_533945.4, SAFB2, G_7, Del, 8, high expression, GATPPTKG, (SEQ ID NO: 5628); XM_014116168.1, XRCC3, G_7, Del, 8, essential, GSPRGPSA, (SEQ ID NO: 5629); XM_014112419.1, POLG, G_7, In, 8, essential, GHRIRNVQ, (SEQ ID NO: 5630); XM_005623182.2, TSHR, G_7, In, 8, driver, EGVSFSPL, (SEQ ID NO: 5631); XM_014107007.1, DIDO1, G_7, In, 9, essential, high expression, AQRSSTTPV, (SEQ ID NO: 5632); XM_005637906.2, TUBGCP2, G_7, Del, 9, essential, high expression, GKGPRSTSR, (SEQ ID NO: 5633); NM_001012395.1, UTRN, G_7, In, 9, high expression, GCPSLTAPV, (SEQ ID NO: 5634); XM_545627.5, CPS1, G_7, In, 9, high expression, AVHWSGRRI, (SEQ ID NO: 5635); XM_535802.5, NDUFS6, G_7, Del, 9, high expression, GLLATRKCT, (SEQ ID NO: 5636); XM_005616765.2, PEPD, G_7, Del, 9, high expression, GSASRRTSW, (SEQ ID NO: 5637); XM_537234.5, PYCR2, G_7, Del, 9, high expression, GPPSMPCTS, (SEQ ID NO: 5638); XM_533945.4, SAFB2, G_7, In, 9, high expression, GLRLRQKDE, (SEQ ID NO: 5639); XM_541491.5, SCAF1, G_7, In, 9, essential, GSGPRQGQR, (SEQ ID NO: 5640); XM_014116933.1, SNAPC4, G_7, Del, 9, essential, GPLTPRDCQ, (SEQ ID NO: 5641); XM_539936.4, MNX1, G_7, Del, 9, driver, GPGISPHPS, (SEQ ID NO: 5642); XM_539936.4, MNX1, G_7, In, 9, driver, GRASAPTPV, (SEQ ID NO: 5643); NM_001003264.1, SRP72, G_7, Del, 10, essential, high expression, GCRCLRSGVK, (SEQ ID NO: 5644); XM_014113182.1, JAK1, G_7, In, 10, driver, high expression, ASRSLHAPEK, (SEQ ID NO: 5645); XM_003433995.4, ADAMTS1, G_7, Del, 10, high expression, GCCLTRAVIL, (SEQ ID NO: 5646); XM_005625506.1, TIMELESS, G_7, Del, 10, essential, GKGLLVRKNS, (SEQ ID NO: 5647); XM_005618879.1, PRF1, G_7, Del, 10, driver, GVPSSACPWH, (SEQ ID NO: 5648); XM_005632217.2, SRGAP3, G_7, In, 10, driver, GDGPDIEKTM, (SEQ ID NO: 5649); XM_847967.4, UBC, G_7, In, 11, essential, high expression, GHADLREDLDG, (SEQ ID NO: 5650); XM_854764.4, MAP1A, G_7, In, 11, high expression, GQWNSPSQAWP, (SEQ ID NO: 5651); XM_535893.4, RANBP9, G_7, In, 11, high expression, GLWEQTKRQNS, (SEQ ID NO: 5652); XM_005630590.1, ADI1, G_7, Del, 11, high expression, GAAGRLRGARW, (SEQ ID NO: 5653); XM_536148.4, ALDH9A1, G_7, In, 11, high expression, GCHRPAPVSAP, (SEQ ID NO: 5654); XM_005624044.2, CYTH1, G_7, In, 11, high expression, GPARGAAPESV, (SEQ ID NO: 5655); XM_005620597.2, NOL9, G_7, In, 11, essential, ACSPSSRAGGK, (SEQ ID NO: 5656); XM_005619964.2, PHF23, G_7, In, 11, essential, GSPSPRAANTP, (SEQ ID NO: 5657); XM_005619566.2, FLI1, G_7, In, 11, driver, DLPQPQRAPPP, (SEQ ID NO: 5658); XM_537686.5, COIL, G_7, Del, 12, high expression, GSCPRPRARAWY, (SEQ ID NO: 5659); XM_005619266.1, LARP1, G_7, Del, 12, high expression, GTAQATTPHVPR, (SEQ ID NO: 5660); XM_538245.6, LRP1, G_7, Del, 12, high expression, GRCIGRTGGQTR, (SEQ ID NO: 5661); XM_005619370.2, C9, G_7, Del, 12, high expression, GAVISGAARADA, (SEQ ID NO: 5662); XM_003435632.3, MORF4L2, G_7, Del, 12, high expression, GVALQRTPLQDQ, (SEQ ID NO: 5663); NM_001287152.1, PIGR, G_7, In, 12, high expression, GYGAVRGQARTA, (SEQ ID NO: 5664); XM_014109402.1, TP53BP1, G_7, In, 12, high expression, GSLCEAAPFKCP, (SEQ ID NO: 5665); XM_005628665.2, JAZF1, G_7, In, 12, driver, GREALRLPGSWM, (SEQ ID NO: 5666); XM_003639638.3, POGZ, G_7, Del, 13, high expression, GPPQLSLKSFRLL, (SEQ ID NO: 5667); XM_014118297.1, MDC1, G_7, Del, 13, essential, GVWPAQWQRLPTW, (SEQ ID NO: 5668); XM_014106951.1, RTEL1, G_7, In, 13, essential, AWGLSVLQGSHPP, (SEQ ID NO: 5669); XM_545865.4, CRTC3, G_7, Del, 13, driver, GLRTPPLDWGRSL, (SEQ ID NO: 5670); XM_845505.2, TLX1, G_7, In, 13, driver, GPERCGGDPGARA, (SEQ ID NO: 5671); XM_014110368.1, LPP, G_7, In, 14, driver, high expression, GHGLCLYATARTSA, (SEQ ID NO: 5672); XM_005631198.2, CTNND1, G_7, Del, 14, high expression, GLHPLTGDSQSCLR, (SEQ ID NO: 5673); XM_005624044.2, CYTH1, G_7, Del, 14, high expression, GTCPRSCSGICMRA, (SEQ ID NO: 5674); XM_005616765.2, PEPD, G_7, In, 14, high expression, GHAPQLLHLHLWQW, (SEQ ID NO: 5675); XM_014118297.1, MDC1, G_7, In, 14, essential, ESGQLSGRGFPLGD, (SEQ ID NO: 5676); XM_005627636.2, MDN1, G_7, In, 14, essential, ATASETDVKGQGRN, (SEQ ID NO: 5677); XM_005639316.1, METTL14, G_7, Del, 14, essential, GAVEEHTEVAFHLD, (SEQ ID NO: 5678); XM_005639316.1, METTL14, G_7, In, 14, essential, GPWRSTQRWLSTSI, (SEQ ID NO: 5679); XM_847066.4, PAXBP1, G_7, Del, 14, essential, GSRCRARGCRRLPR, (SEQ ID NO: 5680); XM_543480.5, PES1, G_7, In, 14, essential, GCILGQVSVHWGYI, (SEQ ID NO: 5681); XM_014112419.1, POLG, G_7, In, 14, essential, ASLREGASRTVRQG, (SEQ ID NO: 5682); XM_005619042.2, ZSWIM8, G_7, Del, 14, essential, GAQGLSRLQWQRQQ, (SEQ ID NO: 5683); XM_005618879.1, PRF1, G_7, In, 14, driver, APEGSGLGPRLWLG, (SEQ ID NO: 5684); XM_014116698.1, RNF43, G_7, Del, 14, driver, GASQSPHQPLSPRT, (SEQ ID NO: 5685); XM_005626080.2, EPAS1, G_7, Del, 15, driver, high expression, GIHQAAALHIRYGKG, (SEQ ID NO: 5686); XM_005636328.2, ACACB, G_7, Del, 15, high expression, GAPGWSWTPPSTPCA, (SEQ ID NO: 5687); XM_533429.5, TXLNB, G_7, Del, 15, high expression, GQGLMPRKSVVFTML, (SEQ ID NO: 5688); NM_001005760.1, LIFR, G_7, Del, 15, driver, GPATSHRCTSPSLLL, (SEQ ID NO: 5690); XM_540482.5, NPLOC4, G_7, In, 16, essential, high expression, GRQGEICCPGEHQLQD, (SEQ ID NO: 5691); XM_014121211.1, TRMT112, G_7, In, 16, essential, high expression, GVVSPPAQSEGRDARR, (SEQ ID NO: 5692); XM_014121803.1, PALM3, G_7, Del, 16, high expression, GQRAATQRPQERRAGA, (SEQ ID NO: 5693); XM_005630813.2, SNX27, G_7, Del, 16, high expression, GRPTGPGCARGTASWR, (SEQ ID NO: 5694); XM_845621.5, MLLT6, G_7, Del, 16, driver, GWRAAGPCPSMGSWGG, (SEQ ID NO: 5695); XM_014117894.1, ABCA1, G_7, Del, 17, high expression, GALPTCKMWWSRRSSGC, (SEQ ID NO: 5696); XM_005636328.2, ACACB, G_7, Del, 17, high expression, GTGSLRRCSSPTMASLP, (SEQ ID NO: 5697); XM_545627.5, CPS1, G_7, Del, 17, high expression, GCPLVRQENLITQDHKL, (SEQ ID NO: 5699); XM_003639638.3, POGZ, G_7, Del, 17, high expression, GLKSHMSGLCVSCSGTT, (SEQ ID NO: 5700); XM_014114365.1, SETD1A, G_7, In, 17, essential, ATPAPYTPFTPFLRTPK, (SEQ ID NO: 5701); XM_005636883.1, KMT2D, G_7, Del, 17, driver, GLHFLVRAPLRDPVFTQ, (SEQ ID NO: 5702); XM_536371.4, TET1, G_7, In, 17, driver, GTEIGIKPTSPRKCNQK, (SEQ ID NO: 5703); XM_536894.5, EIF3B, G_7, In, 18, essential, high expression, GREIQTNPEIQPPRGSAH, (SEQ ID NO: 5704); XM_853836.4, TCERG1, G_7, Del, 18, essential, high expression, GTVSDSTRGSSGWLNSRP, (SEQ ID NO: 5705); XM_014121211.1, TRMT112, G_7, Del, 18, essential, high expression, GSGLTASPERGAGRPEMT, (SEQ ID NO: 5706); XM_532946.5, COX7A2L, G_7, In, 18, high expression, DHLLPDCSLHGFAAQKQM, (SEQ ID NO: 5707); XM_005636883.1, KMT2D, G_7, In, 18, driver, GSISWSGPPSETPFLPSK, (SEQ ID NO: 5708); XM_537686.5, COIL, G_7, In, 19, high expression, ALAPGRERAPGTRQRLPQS, (SEQ ID NO: 5709); XM_005640836.2, MIA3, G_7, Del, 19, high expression, GSAPHLPRLTCLRDRSLLR, (SEQ ID NO: 5710); XM_014106366.1, TBC1D4, G_7, In, 19, high expression, GLPRAQKKASRAHPGRFWP, (SEQ ID NO: 5711); XM_005619566.2, FLI1, G_7, Del, 19, driver, GSTPTPTCPATLTPTCPHT, (SEQ ID NO: 5712); XM_005628665.2, JAZF1, G_7, Del, 19, driver, GKRSPSPARFLDVKRDTRM, (SEQ ID NO: 5713); XM_005619266.1, LARP1, G_7, In, 20, high expression, GPHRQPHLTCQDECRAGQGH, (SEQ ID NO: 5714); XM_005641605.2, TCEAL4, G_7, Del, 20, high expression, GLCGCKKVYRTPSTRGAQGN, (SEQ ID NO: 5715); XM_536612.5, PELP1, G_7, Del, 21, essential, high expression, GPQVRRCCPTCSVISPRRLTL, (SEQ ID NO: 5716); XM_005634379.2, PLCL2, G_7, Del, 21, high expression, GAPPAGPCPPPRARPSAPRAP, (SEQ ID NO: 5717); XM_005630813.2, SNX27, G_7, In, 21, high expression, GGRPGRGAQGGPHPGGERREC, (SEQ ID NO: 5718); XM_544061.5, ZRANB1, G_7, Del, 21, high expression, GCWWPCRRVPGGGTTPWSRRW, (SEQ ID NO: 5719); XM_846693.4, GINS2, G_7, Del, 21, essential, GTWGLLTLVYQWKCLCGWRLT, (SEQ ID NO: 5720); XM_014118328.1, DDAH2, G_7, Del, 22, high expression, GPALWWPVAATQPKRLSGRWQC, (SEQ ID NO: 5721); XM_014114365.1, SETD1A, G_7, Del, 22, essential, GNPRPLHPLHPLPSNPEVSLNR, (SEQ ID NO: 5722); XM_014114199.1, SRRT, G_7, Del, 23, essential, high expression, GLPLRNPLRKGTQQRSMWSEMKN, (SEQ ID NO: 5723); XM_533169.4, MPEG1, G_7, Del, 23, high expression, GFLSIRASPSRPGSRASPTTWWP, (SEQ ID NO: 5724); XM_014119335.1, MYCL, G_7, In, 23, driver, GSRERSPRRGSGERRPRGKGRSG, (SEQ ID NO: 5725); XM_014121803.1, PALM3, G_7, In, 24, high expression, AREQPLRGHRRGGQGPKGCTDQLT, (SEQ ID NO: 5726); XM_532155.4, AARS2, G_7, Del, 24, essential, GAPAAGRSHPSPLRPRPPPCETPS, (SEQ ID NO: 5727); XM_538049.4, SMC1A, G_7, In, 25, essential, high expression, GENSGGSGLALCHPQLQASPLLRPG, (SEQ ID NO: 5728); XM_005626608.1, SILL G_7, In, 25, high expression, AAGPQEPGTGQGHRGAGRACGHSAL, (SEQ ID NO: 5729); NM_001005760.1, LIFR, G_7, In, 25, driver, GRLQATDAPPRHFYCRRPRCRGRPR, (SEQ ID NO: 5731); XM_005630590.1, ADI1, G_7, In, 26, high expression, AQAGGLGDPPRPPCGAQASSAAAVGC, (SEQ ID NO: 5732); XM_536148.4, ALDH9A1, G_7, Del, 26, high expression, GLPPASSCVSTLTWPKSPSPEACPPA, (SEQ ID NO: 5733); XM_541795.5, BHLHE40, G_7, In, 26, high expression, DLQETIRPGSQSHGLQGETQLPGQRL, (SEQ ID NO: 5734); XM_003435632.3, MORF4L2, G_7, In, 26, high expression, GSLCREPPFRISEEDQKEQTEDTWKR, (SEQ ID NO: 5735); XM_548355.4, NDOR1, G_7, Del, 26, essential, GSLALMPLTTLPDSSGHCASRVRHGP, (SEQ ID NO: 5736); XM_014120131.1, SORBS2, G_7, Del, 27, high expression, GNRFRLCITILLGMKMSWSSEKVMSLM, (SEQ ID NO: 5737); XM_535876.5, F13A1, G_7, In, 27, high expression, AGAPRSQPARLSSCYKYSPVQGEMGHQ, (SEQ ID NO: 5738); XM_005616765.2, PEPD, G_7, In, 27, high expression, GPHRGGRRGDRQWHGTADLCAPHGGGD, (SEQ ID NO: 5739); XM_534578.5, SFTPC, G_7, Del, 27, high expression, GQKPSSAWPCRSVWAPLPPSPLAPLAS, (SEQ ID NO: 5740); XM_845621.5, MLLT6, G_7, In, 27, driver, VEWGCRPQPCGLEPGWRGPHAAAARLS, (SEQ ID NO: 5741); XM_014119335.1, MYCL, G_7, Del, 27, driver, GLQREVPKKRLWRETPQRKRKKWMKRL, (SEQ ID NO: 5742); XM_536894.5, EIF3B, G_7, Del, 28, essential, high expression, GERNSNKSRDSATKGFSSLTSHLVKDTW, (SEQ ID NO: 5743); XM_005635420.2, UFM1, G_7, In, 28, high expression, AGVARPRASRRRRRGCRSAGLHDVEGFL, (SEQ ID NO: 5744); XM_005617934.2, PAX7, G_7, Del, 28, driver, GWRRPPQPRTPAQPTEPATASPATRTAS, (SEQ ID NO: 5745); XM_005637906.2, TUBGCP2, G_7, In, 29, essential, high expression, GRGRGLHRDDAEEPDPERDDHRVRAQRQG, (SEQ ID NO: 5746); XM_541696.5, LGI4, G_7, In, 29, high expression, GLPAVGPARPRPAPGAHPSPGPAAAAAAQ, (SEQ ID NO: 5747); XM_534578.5, SFTPC, G_7, In, 29, high expression, ARSPAAPGPAGACGHHCHLLHWLHWHRSV, (SEQ ID NO: 5748); XM_541287.5, TMEM2, G_7, Del, 29, high expression, GTAPRRSWMAFLFITASHGASPCMGQMAC, (SEQ ID NO: 5749); XM_014111168.1, SPEN, G_7, Del, 30, driver, essential, high expression, GEPEEPPPLPTPPPQAAAAAASASAPASRR, (SEQ ID NO: 5750); XM_014118775.1, LIMCH1, G_7, Del, 30, high expression, GPRRHQLPSPKTEMPSKSRSADGWSKLGSE, (SEQ ID NO: 5751); XM_541287.5, TMEM2, G_7, In, 30, high expression, VQPRDVRGWPFYSSQLLTVHHRAWDKWPAD, (SEQ ID NO: 5752); XM_005619964.2, PHF23, G_7, Del, 30, essential, GLALGCLGGLGQLLGTGKRGLELRRARNGS, (SEQ ID NO: 5753); XM_005628314.1, CPSF1, G_7, In, 30, essential, GQEWGSPGARGRGRGAGAAGTPPSGPCTLR, (SEQ ID NO: 5754); XM_005641605.2, TCEAL4, G_7, In, 31, high expression, VYVDAKKFTGPLPPEGPKGTEGWVQGPTEGL, (SEQ ID NO: 5755); XM_003435252.3, CDK12, G_7, In, 31, driver, essential, GPSSIFCLWKALSGAYKSPAKRGKRERSSLL, (SEQ ID NO: 5757); XM_014115404.1, HAX1, G_7, Del, 32, high expression, GSWRVIQEVNPPNQHQTGAPRDLLVCLMICGL, (SEQ ID NO: 5759); XM_005635420.2, UFM1, G_7, Del, 32, high expression, GGGGAAPGEQAASAGVSVRGAPRCRRFPLRSR, (SEQ ID NO: 5760); XM_014109906.1, ANK2, G_7, In, 33, high expression, GCTNLKITMQHNRWNHPSTMGRHYRNYAFNICQ, (SEQ ID NO: 5761); XM_535802.5, NDUFS6, G_7, In, 33, high expression, GSWPPESVHKPGQRNKNGDVRLLWTAVQTSPSL, (SEQ ID NO: 5762); XM_014120941.1, DRAP1, G_7, In, 34, essential, high expression, GGRTVVFGVRTPRPLPCPPGVRVPSPLSRENLSR, (SEQ ID NO: 5763); XM_014120762.1, NAMPT, G_7, In, 34, essential, high expression, GPVLARPERPEVSPRQSRSVPGPGRELERAGYSL, (SEQ ID NO: 5764); XM_014112350.1, UCHL1, G_7, Del, 34, high expression, GFVPVWRRRPRSPSLGVAPAPPAQAPEVRQAGAL, (SEQ ID NO: 5767); XM_005630590.1, ADI1, G_7, Del, 35, high expression, GAGGRPWGPPATPVRSAGLLRSGSWMLTNMRMIQN, (SEQ ID NO: 5768); XM_005618689.2, GAK, G_7, In, 35, essential, GHGAPLHKPQGHLLQGHPVCCQLRKRRFGHLLHHI, (SEQ ID NO: 5769); XM_014121783.1, PTPN23, G_7, In, 35, essential, GRTTPASCRSALCPSSPVLRTRDGPGGSASHDHCG, (SEQ ID NO: 5770); XM_005636328.2, ACACB, G_7, In, 36, high expression, GLLGGRGLLHQPLVHRNVCRQGEQGKYSGARGHSGD, (SEQ ID NO: 5771); XM_005639643.2, CCDC80, G_7, Del, 36, high expression, GATAALPRPRPGRPKSTRSSRSSRSSRSPRRRRGRS, (SEQ ID NO: 5772); XM_537234.5, PYCR2, G_7, In, 36, high expression, GHHPCPALPREWGLPLPAHQCCGGLLHPHTRAAVHG, (SEQ ID NO: 5773); XM_005623182.2, TSHR, G_7, Del, 36, driver, GRGVLLPPVSATRRMTSESPARISTASPPYHPARRL, (SEQ ID NO: 5774); XM_005620597.2, NOL9, G_7, Del, 37, essential, GVLSFFQGGRKVRTRSARNLAGRGPWYRVLLLAGSAV, (SEQ ID NO: 5775); XM_014106952.1, RTEL1, G_7, Del, 37, essential, GLGPVSPTRFTSTLMLVTTGRVLGRMPGAARLPESQS, (SEQ ID NO: 5776); NM_001003016.2, MCL1, G_7, Del, 38, essential, high expression, GPGWGPAAAAPPLREGGFWLRGRRPRPDGREGEGKPVR, (SEQ ID NO: 5777); XM_005626080.2, EPAS1, G_7, In, 38, driver, high expression, GSTRQQHFTSGMEKDEEPQGQRELPFDSRQTAECKHPQ, (SEQ ID NO: 5778); XM_014117894.1, ABCA1, G_7, In, 38, high expression, GLCLLARCGGAGDHQGADGHREENWRLCATDALPLLRR, (SEQ ID NO: 5779); NM_001287152.1, PIGR, G_7, Del, 38, high expression, GLRSSTRAGSHCMKSQAMAPTRSSSTSSPPTMLASTGV, (SEQ ID NO: 5780); XM_005618685.2, GAK, G_7, In, 38, essential, VGGLGRGAGPRSCCRRRPACRPQCPRELDQVSEPGPVC, (SEQ ID NO: 5781); XM_535078.5, LYN, G_7, In, 39, high expression, ESAASKANRLFCPDCRGNGIHREKELHPPRSTSSKRSGF, (SEQ ID NO: 5782); XM_014106951.1, RTEL1, G_7, Del, 39, essential, GLGPVSPTRFTSTLMLVTTGRVLGRMPGAARLPESQGRC, (SEQ ID NO: 5783); XM_543740.5, YARS2, G_7, In, 39, essential, GHRAPRRPQRPPQGARGAGAGAGAGERARPPPRPRGRGG, (SEQ ID NO: 5784); XM_005618689.2, GAK, G_7, Del, 39, essential, GARSASSQTSRTPPPRSSSLLPITQKEIWTSLTSHLELL, (SEQ ID NO: 5785); XM_541491.5, SCAF1, G_7, In, 39, essential, GRGGGGGRGGRRGGGGGGGAAACHHHSHQHSCSGPKHCP, (SEQ ID NO: 5786); XM_535893.4, RANBP9, G_7, Del, 40, high expression, GTLGANKTPEFIARSLHASRSTFARLREGWSRQSQVAKTN, (SEQ ID NO: 5787); XM_005626608.1, SIL1, G_7, Del, 40, high expression, GCRSSGAWYRPRAQRCWPCVWSLCFMTWSLKRCLPRRRLN, (SEQ ID NO: 5788); XM_540482.5, NPLOC4, G_7, Del, 41, essential, high expression, GPTRGNLLPWRTSVARLNQGARDTFRGLTASVLSASQVPSR, (SEQ ID NO: 5789); XM_005626996.1, KLF4, G_7, Del, 41, driver, high expression, GTRAWRRAAAVAAAAAAAASSTAGSPCPLPRPPSTWRTSTT, (SEQ ID NO: 5790); XM_853562.3, R3HDM2, G_7, Del, 41, high expression, GTTVGLLRTAATRTSLPCTPSWLCFPAPWLPRMPPFASTTL, (SEQ ID NO: 5791); NM_001103215.1, COL6A3, G_7, In, 41, high expression, VRPEHWLGPELCPCQPLHRSWRQQDPRTRAAALAPAHSRAV, (SEQ ID NO: 5792); XM_535876.5, F13A1, G_7, Del, 41, high expression, GRCPEESTCKTIFMLQIFTCSRRDGTPIKWTTTLTSMKTTS, (SEQ ID NO: 5793); XM_014112419.1, POLG, G_7, Del, 42, essential, GAQNQKCSISWRALPCLTPHVPRCWAAASAAPWSPRLSRGSL, (SEQ ID NO: 5794); XM_845621.5, MLLT6, G_7, In, 42, driver, AGGQRGPAPQWAPGGVEWGCRPQPCGLEPGWRGPHAAAARLS, (SEQ ID NO: 5795); XM_005630590.1, ADI1, G_7, In, 43, high expression, GPRGACAGRAGSGVHGGAQAGGLGDPPRPPCGAQASSAAAVGC, (SEQ ID NO: 5796); XM_014118775.1, LIMCH1, G_7, In, 44, high expression, AHGGTSSPVRRQRCRRSPEAPTAGASWDQSEAGGAALCQSQAVQ, (SEQ ID NO: 5798); XM_005618689.2, GAK, G_7, In, 44, essential, VGGLGRGAGPRSCCRRSLLLCRRPACRPQCPRELDQVSEPGPVC, (SEQ ID NO: 5799); XM_537649.5, CASC3, G_7, Del, 45, essential, high expression, GPRALRSRNVRVKMASRAMLFCRIMKVQKTLKVKKGSIVKRKTPK, (SEQ ID NO: 5800); XM_854764.4, MAP1A, G_7, Del, 45, high expression, GSVELTIPGLAMTHPLSLNQTLAHPLLALMCAWLTLRGSVQNLEG, (SEQ ID NO: 5801); XM_014106771.1, XIRP1, G_7, Del, 46, high expression, GPASGRRSWCLASFPGSSAKCCAGQMWTSRGCWFRRTQRASSALSH, (SEQ ID NO: 5804); XM_005616873.2, CD3EAP, G_7, Del, 47, essential, GRPPGPERAGSEGHPRAPTPHGRSRTRPRPLVPRGLRRGSSGSGYWG, (SEQ ID NO: 5805); XM_850395.3, USF2, G_7, In, 48, high expression, AAGCDPGGCGWGRPAPRPHCRLCAPRARSALPAGRNPKSLQQWRQPGS, (SEQ ID NO: 5807); XM_005626996.1, KLF4, G_7, In, 49, driver, high expression, GPGRGAGRRRWRRRRRRRPPLQPGARAPSHGPLQPGGHQRREPLGRLRG, (SEQ ID NO: 5809); XM_535634.4, COQ2, G_7, Del, 50, essential, high expression, GCGSWRGRGCGGSAPWPRRARLARSALGTRGLRSGRNRAGVGSAYRPLRW, (SEQ ID NO: 5810); XM_005631208.2, TNKS1BP1, G_7, Del, 50, high expression, GVTSALASSPSPRPPSARASEIGAVTSARRLPREATSLASSVTTGQVVLP, (SEQ ID NO: 5811); XM_543480.5, PES1, G_7, Del, 50, essential, GMYPGTSLCALGLHMMSQTPASPTRLSTGLGSRPLFLAGTMCSLSGCLTV, (SEQ ID NO: 5812); XM_005639643.2, CCDC80, G_7, In, 52, high expression, GRRPRSQGQGQGGQKAREAREAREAREAREGEEEEAEKREGRQVAQGEEGKG, (SEQ ID NO: 5813); XM_014118740.1, EEF1D, G_7, Del, 53, high expression, GAPSWFRWAMASASCRSSAWWRTTRWGQTCWRRRSPSSRSTCRVWTLLLSTRS, (SEQ ID NO: 5816); XM_014118740.1, EEF1D, G_7, In, 53, high expression, GLQAGSGGLWHPQAADPVRGGGRQGGDRPAGGGDHQVRGARAECGHCCFQQDL, (SEQ ID NO: 5817); XM_541491.5, SCAF1, G_7, Del, 55, essential, GIGTETGTEIGTGTGHPRRLGLPRSQRPPRGPRQSHRSAAAQALRRPHAPLPPGR, (SEQ ID NO: 5819); XM_014122111.1, RBM6, G_7, Del, 56, high expression, GEIFFILLISRAEIHHSWTSGVGTYILGIFGIEKDHQWTTGVEMVLLWIIEVERHLT, (SEQ ID NO: 5820); XM_003432584.4, SAMD4B, G_7, Del, 57, essential, GALQSWALGRLGQAGRTSHPGKMDMCPSTHPAQCRQPSTVLGATQMQVSLAKFTPAR, (SEQ ID NO: 5821); XM_536612.5, PELP1, G_7, Del, 60, essential, high expression, GTKCQSPRRRLRQKTWRPRWRQRRQPCRRRSRMTRLPCWLTSSIVPLMTTSHHRPQSLIP, (SEQ ID NO: 5822); XM_014120762.1, NAMPT, G_7, Del, 60, essential, high expression, GPGPGATRAARGQPPPEPQRPRARPGARARRLLTINSTHPIQVKFIPTLNAVKRRQKTPK, (SEQ ID NO: 5823); XM_003432747.3, SRGAP3, G_7, In, 60, driver, GDGP SAVTIRWS GHPQAPKRGRHPQPTPGPGS QHRHATPGRRLP Q QPP QNP SHPGEDREP, (SEQ ID NO: 5824); XM_847066.4, PAXBP1, G_7, In, 63, essential, GVAAGPGAAAAFRADPGLGGRGRGLLPGRRGARQRAEAAQEAAGEQRGAAGQPAELPGRGGRK, (SEQ ID NO: 5826); XM_014112349.1, BOD1L1, G_7, Del, 66, essential, GRGAGVRPAEPSGGERMEDRWPQRWKWKDAARSAWCTAAGKGRVRRALPRVGRGAPRGRTLC QMSE, (SEQ ID NO: 5828); XM_544915.6, PWP2, G_7, In, 68, essential, GRAAGQPGLQVRAPPVPLQGLRAQRLLLSRWQEVRCHKRQPRSDVPRPWEEAGVQRVCPGQNLL WAVR, (SEQ ID NO: 5829); XM_014112350.1, UCHL1, G_7, In, 71, high expression, GS CPFGGGGPVPRRLVLPPLPLRRHPKCVRPALY S RGLGGSARCF S RCLGS GS SRAPPRCS SSRWRS TPRC, (SEQ ID NO: 5830); XM_543461.5, MN1, G_7, In, 71, driver, AAGAAGARILWRPAAAPRPPGRPPPPPTSPPLWGQLRRPGPGGLVPARGSPTWVRRRRRRPGQPAA LRGGL, (SEQ ID NO: 5831); XM_014109910.1, KHDRBS1, G_7, Del, 72, high expression, GAPGPRPPRSHHRCCRPRPRAPTRRWAVRRRPRCCPPQPLPPSRWSRRTSTCPNSWPRRTRSTRPSL TPCSY, (SEQ ID NO: 5832); XM_543740.5, YARS2, G_7, Del, 72, essential, GPPRA S ETPAAAPRSARRWRRS RCGGTRAP SAEA SRPWRLIT SGS SPTGGPGEASRSWTTRPGTRGS TWWTS, (SEQ ID NO: 5834); XM_014109910.1, KHDRBS1, G_7, In, 76, high expression, GRPGLARHAATTAAAALGHGPRRDGGRSGADPAAAPLSHCLRQDGAGEQVPARTHGREGLARPV LHSRHAATDGRN, (SEQ ID NO: 5837); XM_545865.4, CRTC3, G_7, Del, 76, driver, GVWGAGPRAGAGRARRAAVGERRAAARGRPEGEGEASRAPGGNHKQLAGGRRGFSFSAHPPPAA GARRGRGEPGAP, (SEQ ID NO: 5838); XM_533169.4, MPEG1, G_7, In, 78, high expression, GLSGMHPALGEGGRPAVPNLGAEEPPHWCFLLPLWLLPHPPAIPGPRGGLQPPGVPAQVHPPRLLQ DGVRRRVPGGEG, (SEQ ID NO: 5839); XM_014112349.1, BOD1L1, G_7, In, 79, essential, DGGLGCGPPSPPVGRGWRTGGRRDGSGRMRPGVRGARRRGRGESAGRFLAWEEGLRGEGLCAKC QSESGACLPEPEATC, (SEQ ID NO: 5840); XM_538049.4, SMC1A, G_7, Del, 80, essential, high expression, GRKQWRLWPCSLPSTATSQPPS S SWMRSMLPWITPTLARWQITSRSSRLATSRPS SSLSRRS STPRLR ASLESTPSKGTV, (SEQ ID NO: 5841); XM_005616765.2, PEPD, G_7, Del, 83, high expression, GACATAPTPASVAVVRIQLCCTTDTPGPPTTGPSRMETCACSTWVASITASLLTSRARSRPTAS SPRT RRPSMRRCCGAAVLS, (SEQ ID NO: 5842); XM_005624539.1, MSL1, G_7, Del, 86, essential, high expression, GS QRPRAPAAV PDP S IRRCCPFRRAL SWRRPK SLRPGLGTRVGRLP QLPPPRTRRDPHHYLCRGRHP SRPPPPPGPWRPARADGRV, (SEQ ID NO: 5844); XM_533169.4, MPEG1, G_7, Del, 86, high expression, GS FRNAP S S RGRRPP S CAKPWS RGTP SLVL S PAPLATPP STCYPRS TRRATTTW SAGA SAP S S SSARR CAKTCSGWRRLNLGLFGV, (SEQ ID NO: 5845); XM_541491.5, SCAF1, G_7, Del, 86, essential, GQRRRRRKRRKKRRRRRRRSSSLPPPQPPAQLQRPQALPLARGPQPATQGQKMDRPPVSPSCPRCP HPCPGTCLLVWTAPPAVSWP, (SEQ ID NO: 5846); XM_014120941.1, DRAP1, G_7, Del, 87, essential, high expression, GRAHRGVRGTNAPPPSVPSRRPGPQPSLPGEPLSLAPSTPFGDGGDRVRVFCPLPRRGSRRSCKLTK RLGRWRRQCLSSSPGRSSCS, (SEQ ID NO: 5848); XM_014116698.1, RNF43, G_7, In, 87, driver, GPLRAPTSLSAPGLDCTPRLPDFSPLQYQPGIPLVPRGPPTDLWTSRPRQEAATRFFRPLLLKFTASV VVSDSTPVPGSTPTRRGAF, (SEQ ID NO: 5849); XM_005618879.1, PRF1, G_7, In, 89, driver, GCPPAPAPGTDRLARPGLGLPAPRGQGQGQLHRGCGQGSSQQHPQRLARGAGCVSQAQHQCACE HGRLALQGGRLRCPEDPPGQVQLQH, (SEQ ID NO: 5851); XM_014114657.1, DNAJA3, G_7, In, 93, essential, GRGGGFAEPQAERQYLRAFPGRSGLRGTADIDTRCQLQRSKKLPFCLYCLLPHELPFGQRGLLPDTR SAPKCQPEGDQESLLPAGQEIPPGHK, (SEQ ID NO: 5852).

The following are peptides selected for a human cancer vaccine. NM_138270.3, ATRX, A_7, Del, driver, essential, high expression, N/A, KKELENKMKKTQEMRKQKIKSILNQIQILKNLRSQDTDIGFCGTN, (SEQ ID NO: 5856); NM_015001.2, SPEN, A_7, In, driver, essential, high expression, N/A, KKLQIKERKISKLQVSSGQICKSEKCGCCCQSQGGCSTGRGEGIWGGGSLP, (SEQ ID NO: 5857); NM_003913.4, PRPF4B, A_8, Del, essential, high expression, positive array, KKRSINTEVNIRNINIPQKKTRIKNINISINIRNTKEKRLLMLLIKRVCLQQKELNLMI, (SEQ ID NO: 5858); NM_015904.3, EIF5B, A_8, Del, essential, high expression, positive array, KKRKDRRAKNRVLMIMIAKNWKIKIQNQKRLQNRKWKCTLGVMMMMILTNFLKKLKGKLKNQI RSGMGQRRMRITVKKLKSVQE, (SEQ ID NO: 5859); NM_016107.4, ZFR, A_9, In, essential, high expression, positive array, KKKRSCIESLTKYQQQQQFYSWDSKSATL, (SEQ ID NO: 5860); NM_007126.3, VCP, A_9, Del, essential, high expression, positive array, KKMCSSLALPTGLTSLILPSSDLAVLISSSTSHFLMRSPVLPSSRLTCASPQLPRMWTWSSWLK, (SEQ ID NO: 5861); NM_000123.3, ERCC5, A_9, Del, driver, high expression, positive array, KKNYGHCNSPLAFLTQLLPRPTSNPWWMTRRDPFCGGNLISTKLENFVSGISAGTERRQMNLCFLY, (SEQ ID NO: 5862); NM_002714.3, PPP1R10, A_9, Del, essential, high expression, positive array, KKKYCHLRLPSQAPLKGKRAQNQAQPNLLPQNQHHLLRQWTQTVQAPRFPLLKSRSSWIQPLWSQ ELWMPSQWRVLEILTN, (SEQ ID NO: 5863); NM_001090.2, ABCF1, A_10, Del, essential, high expression, positive array, KKSEIPEKAGGRRMWMMMEKRKSSWSVLRSSQCQPVMRRMKYPPQNPAEGRKPRVVMFLQP, (SEQ ID NO: 5864); NM_007214.4, SEC63, A_10, In, essential, high expression, positive array, KKKTYTCAITTVKATETKAGKWSRWE, (SEQ ID NO: 5865); NM_001024847.2, TGFBR2, A_10, Del, driver, high expression, positive array, KKSLVRLSSCVPVALMSAMTTSSSQKNITPAILTCC, (SEQ ID NO: 5866); NM_001271.3, CHD2, A_11, In, positive array, KKILLNLSCQSCTF, (SEQ ID NO: 5867); NM_001199380.1, RNF145, A_11, In, high expression, positive array, KKKKEKKKQHGCKGETGGSVKCGPEGAKHHAVGCPVQMGCQLLFPADPKK, (SEQ ID NO: 5868); NM_001199380.1, RNF145, A_11, Del, high expression, positive array, KKKRKKKTTWLQRRNWRQC, (SEQ ID NO: 5869); NM_001243076.2, E2F3, A_11, Del, essential, positive array, KRGMIRLLVCSPRSSFSS, (SEQ ID NO: 5870); NM_017619.3, RNPC3, A_12, Del, essential, positive array, KKKGLMTLSKMIKKKKNLVI, (SEQ ID NO: 5871); NM_004559.4, YBX1, C_8, In, essential, high expression, positive array, PPRRPRPQRRRHQARHYGQRRRERWPGRPHIGGACRRGQEGHRNEGFGNSKMVQCKERIWFHQQ E, (SEQ ID NO: 5872); NM_001005335.1, HNRNPL, C_8, In, essential, high expression, positive array, PPPTSLRREKDGSTSGGSPSGPKSLRPPVWAPPTPSPTTRVWPSRRQPCAHGLWLGSI, (SEQ ID NO: 5873); NM_001330314.1, SLC35F5, U_10, In, positive array, FFLLCVVFGKFVISRSTFRHTSCYS, (SEQ ID NO: 5874); and NM_018356.2, C4H5orf22, U_8, Del, positive array, FFQSRIPSKKCSLRKSTKSYKSCTNLRNLAPT, (SEQ ID NO: 5875).

The following are selected peptides for a dog cancer vaccine. XM_005637237.2, CHD4, A_7, Ins, essential, driver, high expression, human homologhuman homology, N/A, KGAYALMPAAGGQLWGGAGVCGGGGRGGSSLRQ, (SEQ ID NO: 5876); XM_005641516.2, AURX, A_8, Ins, essential, driver, high expression, human homology, N/A, NKEISRFGMHSSULHGPW, (SEQ ID NO: 5877); XM_005635476.2, PDS5B, A_9, Del, high expression, human homology, positive array, NLDLQHQKKRKKKKDKVEUQNRSQRANSUEHQGEHNRAV, (SEQ ID NO: 5878); XM_005636122.2, SBNO1, A_11, Ins, essential, high expression, human homology, N/A, KEKYRSRFYSKCLISIRSWIKUU, (SEQ ID NO: 5879); XM_846664.4, SEC62, A_11, Del, essential, high expression, human homology, positive array, KMGKRKSLKRRKLQELPKRRKLRKNSNLSHMMIKFFWMEMRYLCGSMUQFULKHLSWD, (SEQ ID NO: 5880); XM_014106746.1, XRN1, A_12, Ins, essential, high expression, N/A, KGNGSLUFENPYIAUFQWL, (SEQ ID NO: 5881); NM_001110767.1, MYH9, C_7, Ins, essential, driver, high expression, N/A, PHLCHHRHRLQEYDARPRGPVHFVHG, (SEQ ID NO: 5882); XM_005639643.2, CCDC80, C_9, Ins, high expression, positive array, HGARAGQGAGAGAGARAGARAGAGAGAGRARPGQPHRPAGARPPGPRGRARAPQARQGQGRPE ERPGQNREQRVRE, (SEQ ID NO: 5883); XM_005621019.2, ACUB, C_10, Del, essential, high expression, N/A, PPQSPPRGHPLKLSPASVSSQAWNPAASMKLPSUPS, (SEQ ID NO: 5884); XM_005637736.2, UCF7L2, C_10, Del, driver, N/A, PQUPSHRLSRPSRCRCP, (SEQ ID NO: 5885); XM_005640152.1, IRF4, C_15, Ins, driver, N/A, PDURSRGPSPASPAAPGPRQQRPLAPURVAPRCPPSSAPGSAPAGF, (SEQ ID NO: 5886); XM_014110360.1, PLD1, G_15, Ins, high expression, N/A, GRCUCSAERLLQVUVSRSRLCQHQRPQHAGKARQRNECACAGHGDGPFGHGRUGVPRWPLRPRP PAAVLQGCPWLSF, (SEQ ID NO: 5888); XM_005640494.1, SF3B1, U_7, Del, essential, driver, high expression, N/A, LNUSGNUEWLWUEEIUDS, (SEQ ID NO: 5889); XM_005618433.2, MORF4L1, U_25, Del, high expression, N/A, LLLQVSEYCAFMGLFFMKQSV, (SEQ ID NO: 5890); XM_850372.4, C4H5orf22, U_8, Del, human homology, positive array, QSRIPSKKCSLRKSUKSYKSCUSLKSLVPU, (SEQ ID NO: 5891); XM_014121460.1, SLC35F5, U_10, Ins, human homology, positive array, LLCVVSGKFVVSRSUFRHUGCYS, (SEQ ID NO: 5892); and XM_536179.5, CHD2, A-11, Ins, high expression, human homology, positive array, ILLNLSSESCUF, (SEQ ID NO: 5893).

The following are selected peptides for another dog cancer vaccine. SMC1A, TAIIGPNGSGCCRIYCHEEPQREDSSI, (SEQ ID NO: 6524); SLAIN2, IPRMQPQASANPCQLLKPMVA, (SEQ ID NO: 6525); ZDHHC17, AVLLMCQMYQPWMCEEYYRLL, (SEQ ID NO: 6526); RBM, GRVIECDVVKGSCQDGEAVHRKPSPGGYRAGDSLTLRAVWEGAGM, (SEQ ID NO: 6527); C4H5orf22, QSRIPSKKCSLRKSTKSYKSCTSLKSLVPT, (SEQ ID NO: 6528); SLC35F5, LLCVVSGKFVVSRSTFRHTGCYS, (SEQ ID NO: 6529); CHD2, ILLNLSSESCTF, (SEQ ID NO: 6530); EIF2B3, QWSSVTSLEWTAQERGCSSWLMKRTWMKSWSLRDPSCRSTQEYISTQALWTPISTA, (SEQ ID NO: 6531); SLC44A4, SPVASRGWVDTLKTPPSTTTGCPSWSPSWGPMSSPAASSVFLACVWTHSSSVSWKTWRGTTARRT GPTTCPKPFSRS, (SEQ ID NO: 6532); PLEKHM2, SSAVAVGGPTPRIGPTPSRSSSPTGRAWS, (SEQ ID NO: 6533); PDS5B, NLDLQHQKKRKKKKDKVETQNRSQRANSTEHQGEHNRAV, (SEQ ID NO: 6534); SEC62, KMGKRKSLKRRKLQELPKRRKLRKNSNLSHMMIKFFWMEMRYLCGSMTQFTLKHLSWD, (SEQ ID NO: 6535); CEP290, NSPNGSRKRKKSCNFRINHGGLEPN, (SEQ ID NO: 6536); ARV1, TQLHFAAESIIIIQLWKALAHSSCHLGA, (SEQ ID NO: 6537); SBNO1, KEKYRSRFYSKCLISIRSWIKTT, (SEQ ID NO: 6538); OPHN1, NLRRMVRGFIPCWIGTYIYLPKRKNLSYKRQTSRWTRKGTISLSPLLIMFIKFRKFRSPRSSTLWSQC WPFFIASSFPTV, (SEQ ID NO: 6539); SWAP70, ISPRVPCSLQKTMHLKYGLFSTFCLRTSIH, (SEQ ID NO: 6540); GAP43, MMRTKRLNKMASNQKIKLIRPRPKFRLASVDT, (SEQ ID NO: 6541); SLITRK3, LLSLLLQASLPMCCDGAGRSCPLEASGKKVWTLLASRCNATGFLRMVEVVEVEVEVGADQLFPPR RRLLRWATYMSTSPTPLPRCATTPSTSLVRRKRWLFPQSRRQGAQNEGVLGHNHQE, (SEQ ID NO: 6542); HNRNPH1, LTAKFKMGLKVFVSSTPEKADRVARLLLNLNQKMKSNWP, (SEQ ID NO: 6543); UNC5D, GWELGSVERMVRLQSRVRAFADSGVHSATPKERGQIL, (SEQ ID NO: 6544); SLC12A3, YLAYREEGEVPELAVHGLAGDAVPRPQTAGHPDPRKPGKRAHLLLP, (SEQ ID NO: 6545); PTPRB, SSRESWEPGGKQQWFHKVPGSELVAPCWRLGAVSDPALQ, (SEQ ID NO: 6546); CLEC16A, RDRDGDHGARQAPRVGSQHLCAGTEHYGRREECRCHGLGECAVEQTLPRYGVPRPGQPR, (SEQ ID NO: 6547); TMEM219, GLGLFLEIFWPADPVPREWDSHREVAWPSRRGLTDHFELWRWSRREQDTDIPSHGPG, (SEQ ID NO: 6548); AGPAT4, QAALQKDQLPTILLCLQPAGDVAGVVVGHGVRHPHRPPGLPQVWKGKCHCGSEPQV, (SEQ ID NO: 6549); PICALM, DHQTPLVLYQEHSCRQHPAPPVTVLTELQERTLLQSSLWRISYKSL, (SEQ ID NO: 6550); ECHDC1, GLSHTAGEAAARTPRPGCKGCPPGGRGGLPPSPLLRVLYETWEDASSSPGN, (SEQ ID NO: 6551); ST6GALNAC3, EGNWERQSPVWLLSQHGMVYLHSGHGCLLWYSRLRDDK, (SEQ ID NO: 6552); NTAN1, APSNPSPTTLRVEGWRCTSWEASVTTGSCHKNLLTSFLVNLTDKRRTFT, (SEQ ID NO: 6553); PLAGL2, WPPTQPRNPTSVCTVIRCFIARTICGITCRPMTPTKRPFTVLSAVRITIRS, (SEQ ID NO: 6554).

The following peptides are the total human MS antigens on the array and their positive rate in 89 stage I breast cancer patients compared to 103 age-matched healthy people. The cutoff for the positive of each peptide is the average signal plus 2 fold standard deviations of the healthy people signal. EIF5B, Del, A8, RKDRRAKNRVLMIMIAKNWKIKIQNQKRLQNRKWKCTLGVMMMMILTNFLKKLKGKLKNQIRSG MGQRRMRITVKKLKSVQE, 82, essential, high expression, 12.4, % positive cancer, 9.7, % positive normal, (SEQ ID NO: 5893); HNRNPL, Del, C_8, HLTTKGEGWVHQWGVTVGAQVATAPSMGTPHPLPHHPSMALTPTALCSWSMAWINLR, 57, essential, high expression, 11.2, % positive cancer, 8.7, % positive normal, (SEQ ID NO: 5894); PRPF4B, Del, A_8, RSINTEVNIRNINIPQKKTRIKNINISINIRNTKEKRLLMLLIKRVCLQQKELNLMI, 57, essential, high expression, 10.1, % positive cancer, 6.8, % positive normal, (SEQ ID NO: 5895); HNRNPL, Ins, C_8, PTSLRREKDGSTSGGSPSGPKSLRPPVWAPPTPSPTTRVWPSRRQPCAHGLWLGSI, 56, essential, high expression, 7.9, % positive cancer, 9.7, % positive normal, (SEQ ID NO: 5896); OGDH, Ins, U_8, SQHECRSPTGHCLPESPSPEPRLPGCCGPCTVPGRSTAQRGQARGGPPGSAVAHQGISDTRAPCSTA GPPGDFGC, 75, essential, high expression, 2.2, % positive cancer, 8.7, % positive normal, (SEQ ID NO: 5897); YBX1, Ins, C_8, RRPRPQRRRHQARHYGQRRRERWPGRPHIGGACRRGQEGHRNEGFGNSKMVQCKERIWFHQQE, 63, essential, high expression, 9, % positive cancer, 11.7, % positive normal, (SEQ ID NO: 5898); AGT, Del, U_8, NSIFLSLKRMRESPQSLPNSLTSLRSWR, 28, high expression, 7.9, % positive cancer, 9.7, % positive normal, (SEQ ID NO: 5899); DIAPH1, Del, C_8, HLLPCLEKQECHLLLPLFLVVLESLHLLHFPEALAFLHLHPEWVCLHLPHLDLEFLQPQFCHLD, 64, high expression, 14.6, % positive cancer, 6.8, % positive normal, (SEQ ID NO: 5900); GPATCH4, Del, A8, RKRKGGRKRRKKLQHLKGMMQMRSTQNMLSRTSEKARRRKGDIKKERSQMKERVQLKGMRRRT LQEQVGLGN, 72, high expression, 13.5, % positive cancer, 12.6, % positive normal, (SEQ ID NO: 5901); PHACTR2, Del, A_8, IPRLLVSRPPSLHPSQQAETRPERLLAPLIQKKQLALKHQLRHPLHPPHLVPKLQRRQFLAKQGQWG PPRAREKLTSSQ, 79, high expression, 11.2, % positive cancer, 13.6, % positive normal, (SEQ ID NO: 5902); PJA1, Del, U_8, ILMMMTICHTVLPGGGIPPMTMRATRMAWQEEGEARVQVAIPSQSTLKTNGKRGVTK, 57, high expression, 5.6, % positive cancer, 7.8, % positive normal, (SEQ ID NO: 5903); PLK2, Del, U_8, CRASLRTDCLLAVVIQFQISTYQAQLRISLRKQLLLFLVAKKTKQDILTHIIECLKKMKTSTSLGMI, 67, high expression, 10.1, % positive cancer, 12.6, % positive normal, (SEQ ID NO: 5904); PSD3, Del, A_8, SLPQKVLRRKLTEHIQRPSVVLEVLLTHFWTFLMIQMLLCTKVDSWLGKFMQIWMERRLQEENED GKPFMLY, 72, high expression, 10.1, % positive cancer, 9.7, % positive normal, (SEQ ID NO: 5905); RAPGEF2, Del, A_8, FFLCLKKEVWNVTRNRLKIQYQMHLRSFLULLLHRVLQGKAILWLPVVLWIIFQILVTVKFLHDPV LLAIRLLTQCQSHCTMRGARGILSASWKQT, 97, high expression, 7.9, % positive cancer, 5.8, % positive normal, (SEQ ID NO: 5906); SORBS2, Del, A_8, IRLLRNRLKRQNVKHLCLRLWTLPLKTSVTKLKLKRKGGACRTTASCTASSVSCCQMFPRGTHP, 64, high expression, 12.4, % positive cancer, 10.7, % positive normal, (SEQ ID NO: 5907); VEGFA, Del, A_8, QFEERERGKNESARNPGISPGAFPVGLAQSGESICLYKIRRRVNVPAKTQTRVARRGSLS, 60, high expression, 12.4, % positive cancer, 15.5, % positive normal, (SEQ ID NO: 5908); DIAPH1, Ins, C_8, PTSSLAWRSRNATSSSPSSWWSWNPSTSSISRRPWHSSTSTRNGYASTSPIWIWSSCSPSSAIWINPQK AL, 71, high expression, 15.7, % positive cancer, 9.7, % positive normal, (SEQ ID NO: 5909); HDGF, Ins, U_8, FRDPRDGIPGPQRPLPLRGIQGEVWQAQQEERVQRGAVGDREQPYCQGFRLSVLPEKELCGRA, 63, high expression, 6.7, % positive cancer, 10.7, % positive normal, (SEQ ID NO: 5910); RHOQ, Ins, A_8, TVKKNRIKMYKLLFNYV, 17, high expression, 7.9, % positive cancer, 5.8, % positive normal, (SEQ ID NO: 5911); SHC1, Ins, G_8, GGRHEASGRSRSRGCSTHCTQCPDPQPLGSYIACRTACWGRSRSPQTDATSTTLSSRQRAF, 61, high expression, 7.9, % positive cancer, 7.8, % positive normal, (SEQ ID NO: 5912); SMARCC2, Ins, C_8, PWTPWPLTVPQPTNSSLNDARGSARQRAPRRGGPKPCHCGSCSGQPPAQCQPTARPRHPPASRPHS PEPRHGHPCATSTV, 80, high expression, 11.2, % positive cancer, 13.6, % positive normal, (SEQ ID NO: 5913); BAG6, Del, C_8, QLPMQRHLPLVLGRPHPWLRLLPMSSPQLRGLPRQVQLPRQPPATRGSSGFPTRVWNPWS, 60, essential, 7.9, % positive cancer, 5.8, % positive normal, (SEQ ID NO: 5914); DIS3, Del, A_8, FMRNFLNMLCFENILLHLHQIMKFLLRQPGQGIWKLRLIQPSLWLSLWIRPNLLLFHI, 58, essential, 5.6, % positive cancer, 7.8, % positive normal, (SEQ ID NO: 5915); MDN1, Del, A_8, RSLLYSRMCLVQIPTHTWEPDYFVSLPMMFSWATQSFPVGAVFLTRPAIPCCSCTSHSNPWSQS, 64, essential, 10.1, % positive cancer, 7.8, % positive normal, (SEQ ID NO: 5916); SRCAP, Del, C_8, HHLVPLFIWTPWRKSGSGSGLNAWNGFSNLVRLMGPWHLCMGLKSWISVPCPNLLPAPSALVLLA PATPPFGLIPRLPTGLYCFSPSD, 88, essential, 10.1, % positive cancer, 12.6, % positive normal, (SEQ ID NO: 5917); SRFBP1, Del, A_8, TKDLHHSLVTVMAEKNFVKRRRNILMIAQKKGFTSSLPCLKIVIAVTTSSLGKSDGHERRKVVVIL QLRNKNH, 74, essential, 10.1, % positive cancer, 9.7, % positive normal, (SEQ ID NO: 5918); TOPBP1, Del, A_8, SSKDKNLALLREHLVGGRLFYMWISLEKQASNAFFSQEEQRCYLVILYLYLKRPHIFFLT, 60, essential, 10.1, % positive cancer, 10.7, % positive normal, (SEQ ID NO: 5919); TTF1, Del, A_8, SLRKKRRKSPITRNLRHWPCLKDFIKWAVRLGLICRNPGLLWACMVKLQEYQHLLIKTSLRKKRKS PITRNLRQWPCLRASRVHTLKDHRWAVRLGLWKAVQLLKGSRNPTVQRRSLRKGSLRLSKGHECL VMIFQCPVRTLRAHSLIQ, 148, essential, 15.7, % positive cancer, 9.7, % positive normal, (SEQ ID NO: 5920); PPRC1, Ins, C_8, PTNGAPSVWYSWCLCRASHLQCALGTPSCPSLTLQFHMYLWALGMGPRASTCSILVYCSPTSFASS LHWESCSPT, 75, essential, 14.6, % positive cancer, 11.7, % positive normal, (SEQ ID NO: 5921); ASXL1, Del, G_8, VARVEVAAGPPMREVAEAAAVVMVVRPVATLSPGEARAPLESVRQIYSEHNYCRLIL, 57, driver, 7.9, % positive cancer, 8.7, % positive normal, (SEQ ID NO: 5922); SPECC1, Del, A_8, TYMEMHYGHQAPQVAMLPKLLCRQMLPTLSTLQQRHPQGPCPPPVTPLRVQSVLLLGVPQTA, 62, driver, 4.5, % positive cancer, 6.8, % positive normal, (SEQ ID NO: 5923); TFE3, Ins, G_8, GTCPECSPSAAPCTALRCPSGPALSQRPPGGPGRPLPPGAGGHSDGGGGGGGGRTVGGCPVPTAGC LRSPALFSVPCCLQGQQPPQQLQHGRGVL, 95, driver, 2.2, % positive cancer, 12.6, % positive normal, (SEQ ID NO: 5924); BPTF, Del, A_9, KRRNRKKKKRCSKRHG, 16, essential, high expression, 3.4, % positive cancer, 2.9, % positive normal, (SEQ ID NO: 5925); DYNC1I2, Del, A_9, KKQTRRRKLLLLCKKNQILKKKGEKLKHCFKAWG, 34, essential, high expression, 3.4, % positive cancer, 6.8, % positive normal, (SEQ ID NO: 5927); PPP1R10, Del, A_9, KYCHLRLPSQAPLKGKRAQNQAQPNLLPQNQHHLLRQWTQTVQAPRFPLLKSRSSWIQPLWSQEL WMPSQWRVLEILTN, 79, essential, high expression, 15.7, % positive cancer, 13.6, % positive normal, (SEQ ID NO: 5928); SEC62, Del, A_9, MVKRKNPKRRKLQELLKRRKLRKNSNLSHMMIRFFWMEMRCMYGSMTQFTLKHLSWD, 57, essential, high expression, 14.6, % positive cancer, 10.7, % positive normal, (SEQ ID NO: 5930); VCP, Del, A_9, MCSSLALPTGLTSLILPSSDLAVLISSSTSHFLMRSPVLPSSRLTCASPQLPRMWTWSSWLK, 62, essential, high expression, 16.9, % positive cancer, 13.6, % positive normal, (SEQ ID NO: 5931); VEZT, Del, U_9, LSAIRKMTYFTSWILDSDSTHYIPSCNRKSCYKRMWS, 37, essential, high expression, 5.6, % positive cancer, 5.8, % positive normal, (SEQ ID NO: 5932); BPTF, Ins, A_9, KREETGRRRNDAASDMGKIHISS, 23, essential, high expression, 3.4, % positive cancer, 5.8, % positive normal, (SEQ ID NO: 5933); DYNC1I2, Ins, A_9, KRNRPEEGSCCSCARRIRS, 19, essential, high expression, 7.9, % positive cancer, 5.8, % positive normal, (SEQ ID NO: 5934); ZFR, Ins, A_9, KRSCIESLTKYQQQQQFYSWDSKSATL, 27, essential, high expression, 7.9, % positive cancer, 3.9, % positive normal, (SEQ ID NO: 5935); ACBD3, Del, A_9, GRRKRSEGGVKRKKENVCKRRKRNVGEKKRKGFDGRKRKGDG, 42, high expression, 4.5, % positive cancer, 4.9, % positive normal, (SEQ ID NO: 5937); ASH1L, Del, A_9, QRKVVKNPSQPVPLRNDGITNGNDSTRSCSISLKKSLEKMPLM, 43, high expression, 1.1, % positive cancer, 5.8, % positive normal, (SEQ ID NO: 5938); CIR1, Del, A_9, YKGRKERKTSVQGITTVILKRRTSLRRESFMKNFLAVTITGKKPRKSPGS, 50, high expression, 7.9, % positive cancer, 9.7, % positive normal, (SEQ ID NO: 5939); ERCC5, Del, A_9, NYGHCNSPLAFLTQLLPRPTSNPWWMTRRDPFCGGNLISTKLENFVSGISAGTERRQMNLCFLY, 64, driver, high expression, 7.9, % positive cancer, 6.8, % positive normal, (SEQ ID NO: 5940); HDAC2, Del, A_9, KSATTTTVILEIIIMDRVIP, 20, high expression, 7.9, % positive cancer, 3.9, % positive normal, (SEQ ID NO: 5941); LMAN1, Del, A_9, KRNSRRATPTSKGSLRRKYLRV, 22, high expression, 6.7, % positive cancer, 1.9, % positive normal, (SEQ ID NO: 5942); PDS5B, Del, A_9, NLDLQHQRRRKKKKDKVEIRNRSPKANSTECQGEHSREQNLLNLVQLNPHSPHHRKDEEDHQKRH HHHNQKKMSV, 75, high expression, 11.2, % positive cancer, 16.5, % positive normal, (SEQ ID NO: 5943); PRDM2, Del, A_9, KFLIHLRKVDTHHLQVVTKTVTATTADGQRMRRLKCKACRLRWARPEPAAQAPPKSHFPPHPSGP SRTSSLQLR, 74, high expression, 6.7, % positive cancer, 7.8, % positive normal, (SEQ ID NO: 5944); SPAG9, Del, A_9, GQAFGSFSADFSAPQVTRLRSLNHLLI, 27, high expression, 2.2, % positive cancer, 4.9, % positive normal, (SEQ ID NO: 5945); TCF25, Del, A_9, NRKTRKAAREKHRKTD, 16, high expression, 1.1, % positive cancer, 3.9, % positive normal, (SEQ ID NO: 5946); TNKS2, Del, A_9, TGMEILLWILLKMEIQIFKICLGEMQLC, 28, high expression, 6.7, % positive cancer, 3.9, % positive normal, (SEQ ID NO: 5947); UGDH, Del, U_9, LPILMMPSKKLILYLFLCCPTLSFWQREQPSRT, 33, high expression, 6.7, % positive cancer, 7.8, % positive normal, (SEQ ID NO: 5948); ZC3H11A, Del, U_9, SIPHVPKVTAAHSVTVKLQ, 19, high expression, 9, % positive cancer, 2.9, % positive normal, (SEQ ID NO: 5949); ABCC5, Ins, U_9, FVYVCDAVAGCAAGPHQHRPHHHHGADDRSYARADSPSLCGSRHLLCCPVNGAVPVYGQTGI, 62, high expression, 9, % positive cancer, 7.8, % positive normal, (SEQ ID NO: 5950); ANO10, Ins, A_9, KRWSEKHHVWHRDSGVSTA, 20, high expression, 7.9, % positive cancer, 3.9, % positive normal, (SEQ ID NO: 5951); ARV1, Ins, A_9, TAKKAQLHFAAESIIII, 17, high expression, 2.2, % positive cancer, 2.9, % positive normal, (SEQ ID NO: 5952); CHD2, Ins, A_9, KTEEEREQGEQGETNEF, 17, high expression, 6.7, % positive cancer, 2.9, % positive normal, (SEQ ID NO: 5953); COBLL1, Ins, U_9, FADAEESIGSLCDICSCQECPCCP, 24, high expression, 2.2, % positive cancer, 1.9, % positive normal, (SEQ ID NO: 5954); FAM60A, Ins, A_9, KLESCGRCKGWTQSKDYIETKESENSIWEQDKKQPDQ, 37, high expression, 10.1, % positive cancer, 7.8, % positive normal, (SEQ ID NO: 5955); ITSN1, Ins, U_9, FSIWVTSTCFSTDMGTS, 17, high expression, 2.2, % positive cancer, 4.9, % positive normal, (SEQ ID NO: 5956); LMAN1, Ins, A_9, KRGIPEGPPRPPRAACGGNI, 20, high expression, 1.1, % positive cancer, 5.8, % positive normal, (SEQ ID NO: 5957); MS4A6A, Ins, U_9, FYHLWLSINRHREKVNQAFGA, 21, high expression, 4.5, % positive cancer, 3.9, % positive normal, (SEQ ID NO: 5958); PDS5B, Ins, A_9, KIWTSSTRGGGRRRKTKWKYGTEVQKQTAPSVKESTAESRIS, 42, high expression, 3.4, % positive cancer, 4.9, % positive normal, (SEQ ID NO: 5959); RBM26, Ins, A_9, KTGGIETSAGCKEKETRNFRKAH, 23, high expression, 2.2, % positive cancer, 3.9, % positive normal, (SEQ ID NO: 5960); RYR2, Ins, U_9, FCRSPSRHCYGIQDIKNHLVLSNSQWQTARINRWLISCCCIPIHCGGIQFFPKILQ, 56, high expression, 12.4, % positive cancer, 10.7, % positive normal, (SEQ ID NO: 5961); SLC23A2, Ins, C_9, PHPRNKQGNFRGRPLLCS, 18, high expression, 1.1, % positive cancer, 4.9, % positive normal, (SEQ ID NO: 5962); SPAG9, Ins, A_9, KKVKHLAVFQPTFQLLK, 17, high expression, 2.2, % positive cancer, 4.9, % positive normal, (SEQ ID NO: 5963); TCF25, Ins, A_9, KTEKQEKQHGRSIGKRTRRYRSHPRED, 27, high expression, 1.1, % positive cancer, 4.9, % positive normal, (SEQ ID NO: 5964); ZMPSTE24, Ins, U_9, FICCINWSKGAFCCIWFL, 18, high expression, 4.5, % positive cancer, 4.9, % positive normal, (SEQ ID NO: 5965); ZNF644, Ins, A_9, KKAKNGCKQDNSLYRGLL, 18, high expression, 7.9, % positive cancer, 3.9, % positive normal, (SEQ ID NO: 5966); CDC7, Del, A_9, TSRILNLQVLKKILRSFMKLYHSLVMCLRLRTKLEKALSALFIWPQHSYK, 50, essential, 13.5, % positive cancer, 9.7, % positive normal, (SEQ ID NO: 5967); CEP57, Del, A_9, CKSWKQNSMKKNRKGNACKLRQLSCRLV, 28, essential, 2.2, % positive cancer, 5.8, % positive normal, (SEQ ID NO: 5968); EIF2B3, Del, A_9, KQWSSVTSLEWTAQERGCSSWLMKQTWMKSWSLRDPSYRSILEYVSTRVLWMPTSTV, 57, essential, 7.9, % positive cancer, 13.6, % positive normal, (SEQ ID NO: 5969); GTF3C3, Del, A_9, LQKKAPQKRIKLLRMLPALYLMACQ, 25, essential, 7.9, % positive cancer, 5.8, % positive normal, (SEQ ID NO: 5970); MARS, Del, U_9, CYLAGSKMTSLTSGWNGKRQSCSQLCLLPCTI, 32, essential, 15.7, % positive cancer, 10.7, % positive normal, (SEQ ID NO: 5971); SFSWAP, Del, A_9, NQESALTMKMMMMKKMGITFIPLSLPPRSVTALKS, 35, essential, 16.9, % positive cancer, 7.8, % positive normal, (SEQ ID NO: 5972); CHEK1, Ins, A_9, NIPQPLEKNRFCSSSSAA, 18, essential, 5.6, % positive cancer, 3.9, % positive normal, (SEQ ID NO: 5973); SMC6, Ins, A_9, NILVKRESKGLSKSRKFSQSRDRTVSASHRKGQRRTWQN, 39, essential, 10.1, % positive cancer, 10.7, % positive normal, (SEQ ID NO: 5974); KMT2C, Del, A_9, RNKKTKLWFSLINIHHRKNPLLPMR, 25, driver, 3.4, % positive cancer, 2.9, % positive normal, (SEQ ID NO: 5975); MLLT10, Del, A_9, NIKRRTNTNRNTRSSQNHHLHWFHP, 25, driver, 13.5, % positive cancer, 2.9, % positive normal, (SEQ ID NO: 5976); TCF7L2, Del, A_9, SAFATYKVKAAASAHPLQMEAY, 22, driver, 2.2, % positive cancer, 4.9, % positive normal, (SEQ ID NO: 5977); TCF7L2, Ins, A_9, KCVRYIQGEGSCLSPPSSDGSLL, 23, driver, 2.2, % positive cancer, 1.9, % positive normal, (SEQ ID NO: 5978); ABCF1, Del, A_10, SEIPEKAGGRRMWMMMEKRKSSWSVLRSSQCQPVMRRMKYPPQNPAEGRKPRVVMFLQP, 59, essential, high expression, 14.6, % positive cancer, 10.7, % positive normal, (SEQ ID NO: 5979); SEC63, Del, A_10, NLHLCYYHSQSNRNKSRQMESLGMKLQ, 27, essential, high expression, 4.5, % positive cancer, 1.9, % positive normal, (SEQ ID NO: 5980); SEC63, Ins, A_10, KTYTCAITTVKATETKAGKWSRWE, 24, essential, high expression, 9, % positive cancer, 1.9, % positive normal, (SEQ ID NO: 5981); WNK1, Ins, A_10, KAGREQSQTAGRTIQCFPDRNQAAPFC, 27, essential, high expression, 2.2, % positive cancer, 4.9, % positive normal, (SEQ ID NO: 5982); PARP14, Del, A_10, ITHPFASQPLGQEMPNNTQIRLLKP, 25, high expression, 2.2, % positive cancer, 6.8, % positive normal, (SEQ ID NO: 5983); SHC1, Del, U_10, SCQLNLGRKEHAKIFTFFFQLDTMDGNPGELTLELQTLQIKQSQNALLPAGPLTQTPV, 58, high expression, 14.6, % positive cancer, 11.7, % positive normal, (SEQ ID NO: 5984); SLC35F5, Del, U_10, FALCGFWQICHIKKHFQTHKLL, 22, high expression, 6.7, % positive cancer, 2.9, % positive normal, (SEQ ID NO: 5985); TGFBR2, Del, A_10, KEKKSLVRLSSCVPVALMSAMTTSSSQKNITPAILTCC, 38, driver, high expression, 29.2, % positive cancer, 16.5, % positive normal, (SEQ ID NO: 5986); PARP14, Ins, A_10, KLLIHLPPSHWDRKCQTTPR, 20, high expression, 4.5, % positive cancer, 4.9, % positive normal, (SEQ ID NO: 5987); SLC35F5, Ins, U_10, FLLCVVFGKFVISRSTFRHTSCYS, 24, high expression, 3.4, % positive cancer, 1.9, % positive normal, (SEQ ID NO: 5988); RFC3, Del, A_10, KLKLAPLQVTTTLKLILVMLEIVTE, 25, essential, 10.1, % positive cancer, 4.9, % positive normal, (SEQ ID NO: 5989); GART, Ins, A_10, KGQSGCLNIWNRIEPASTYRQYSGTK, 26, essential, 5.6, % positive cancer, 2.9, % positive normal, (SEQ ID NO: 5990); AASDH, Del, U_10, WDEKTVRSNVMANVLTLNLCNRLLKSFSKWSLVQLHGIIRKN, 42, 6.7, % positive cancer, 6.8, % positive normal, (SEQ ID NO: 5991); KIAA2018, Del, A_11, AKKKTLRNHLFLIRWIILTFLQKILK, 26, high expression, 5.6, % positive cancer, 7.8, % positive normal, (SEQ ID NO: 5992); RNF145, Del, A_11, KRKKKTTWLQRRNWRQC, 17, high expression, 5.6, % positive cancer, 4.9, % positive normal, (SEQ ID NO: 5993); MARCKS, Ins, A_11, PKKKEALFLQEVFQAERLLLQEEQEGGWRRR, 31, high expression, 3.4, % positive cancer, 6.8, % positive normal, (SEQ ID NO: 5994); RNF145, Ins, A_11, KKEKKKQHGCKGETGGSVKCGPEGAKHHAVGCPVQMGCQLLFPADPKK, 48, high expression, 16.9, % positive cancer, 16.5, % positive normal, (SEQ ID NO: 5995); E2F3, Del, A_11, PKKKRGMIRLLVCSPRSSFSS, 21, essential, 9, % positive cancer, 4.9, % positive normal, (SEQ ID NO: 5996); MIS18BP1, Del, A_11, VEKKNCSIPTYVKKRKTTNHSSQMTVH, 27, essential, 9, % positive cancer, 4.9, % positive normal, (SEQ ID NO: 5997); TAF1B, Del, A_11, LKKKTILKKAGIGMCVKVSSIFFINKQKP, 29, essential, 11.2, % positive cancer, 6.8, % positive normal, (SEQ ID NO: 5998); RNPC3, Del, A_12, EKKKGLMTLSKMIKKKKNLVI, 21, essential, 2.2, % positive cancer, 4.9, % positive normal, (SEQ ID NO: 5999); AXIN, Del, G_7, HLWGATAGTPAPPPVPTCSPRTLRCLP, 27, 9, % positive cancer, 8.7, % positive normal, (SEQ ID NO: 6000); AXIN, Ins, G_7, HLWGGQQRAPPHHPPCPPVHPGPCDASPDPTQHAGSAGGGLSQAS, 45, 21.3, % positive cancer, 18.4, % positive normal, (SEQ ID NO: 6001); ADAM30, Del, A_8, IASIAQSCSLTVCLRNAIPGVFATTEKTATACMGGHLHSVRKWGMEEALTVGLQDCSEGRFPRQFG LCPS, 70, 12.4, % positive cancer, 14.6, % positive normal, (SEQ ID NO: 6002); ADAMTSL4, Del, G_8, VAPRCPRSAVDISPGPTSPSLASCASVAIGKLALLGASAPCGAAGAREAGRFAVLGTMVMK, 61, 6.7, % positive cancer, 9.7, % positive normal, (SEQ ID NO: 6003); ASXL3, Del, A_8, CYLHHWNYLSFLKGQIIREMSFHLLNYRTSNISHQWIRLHFQKALEIKHISKGVHRVG, 58, 7.9, % positive cancer, 8.7, % positive normal, (SEQ ID NO: 6004); BAX, Del, G_8, RMGGRHPSWPWTRCLRMRPPRS, 22, 3.4, % positive cancer, 6.8, % positive normal, (SEQ ID NO: 6005); BEGAN, Del, G_8, TGTGSGCSCVGPPAALSPSRVPGTPWSRAPWRPPRKCILPPASAPSRPFRGLVARG, 56, 1.1, % positive cancer, 6.8, % positive normal, (SEQ ID NO: 6006); CASKN2, Del, C_8, RRGTSQRAQSGPLSFVLHFLAKDPHPMFLCTPRAHPLARPQGHLLAHPGPSPTWPGPLPLPQTRLDL SAGPTA, 73, 10.1, % positive cancer, 13.6, % positive normal, (SEQ ID NO: 6007); CEL, Del, C_8, CRPRVTPGPPPCRPRVTPGPPPCRPRVTPGPPPCRPRVTPGPPPCRPRVTPGPPPCRPRVTPGPPPCRPR VTPAPPPCRPRVTPGPPPCRPRVTPAPPPCRPRVTPGPPP, 110, 4.5, % positive cancer, 7.8, % positive normal, (SEQ ID NO: 6008); CEP76, Del, A_8, LILIQHGGIFTFRFWVEKLSWNICKNLSLYLDKFVQRLLYVYIIETNVFVLNLFHVPVNQIFMMAFYL KYTEKAWVMELEWLIQQQCYQ, 89, 10.1, % positive cancer, 9.7, % positive normal, (SEQ ID NO: 6009); CLCA4, Del, A_8, SKQQGVPQVSLVEIEFISVKEAAVLVEHAELILQQNCMEKIVNSFLIKYKQKKHP, 55, 12.4, % positive cancer, 11.7, % positive normal, (SEQ ID NO: 6010); CNTN6, Del, A_8, SLLFKLVGILLSDANQMLFPGQLSLGKEERRPLDKAKEYFSWRMAASRYIILPGQMLDHIHA, 62, 14.6, % positive cancer, 4.9, % positive normal, (SEQ ID NO: 6011); CXXC4, Del, G_8, VAAAAAAAEAGAGVVGAAGNPPPPPPPPPPPPPRRSSPPAVAAAAAAAAAGPACTTETTPRGWGK LAARQSRRCKWQILISSPPYPLNTADLWRGNA, 97, 14.6, % positive cancer, 13.6, % positive normal, (SEQ ID NO: 6012); FOXN2, Del, A_8, QRLQSPHTPLVFSFIWPLSTLQINVCLSKKFIAGFWTIFHILLLHQQAGRILFDIICP, 58, 11.2, % positive cancer, 8.7, % positive normal, (SEQ ID NO: 6013); FRA1OAC1, Del, A_8, EKIKPKKTVKSHHIKNPDYLLQKRPPRKKIKDIHLQRNLKILYLETLMRKKVLQNLNFGRVHYQRQ MKNHRKKNLMSIFRICFY, 84, 15.7, % positive cancer, 9.7, % positive normal, (SEQ ID NO: 6014); HOXB7, Del, G_8, AWRARARPASTRPAMGSSRVPSTCTARPLSRTSPGCVPATPPRRRAPRSRGTRTWRPRVTSGSTPGC EAQELTANEAARPTPATRPWSWRKNFTTIAT, 98, 11.2, % positive cancer, 10.7, % positive normal, (SEQ ID NO: 6015); IRX5, Del, C_8, APAGPPRLGQRRRGWRRTRPLTTPPERRRPARIQPRARCLRVPAGPRLSIRRLRRRLLRCSPSPNCGL WQRSPHRRTRSRTGAAGTRALHAHRVPGP, 97, 11.2, % positive cancer, 10.7, % positive normal, (SEQ ID NO: 6016); LMTK3, Del, C_8, RGTRTRQRLQRPRHLPTPPPPEMGFPATTAALEAVSSGRRISPSSPLQAPRCASPASPSRLRWRPRGH PPGPPTPGPQAPWRI, 83, 12.4, % positive cancer, 8.7, % positive normal, (SEQ ID NO: 6017); MEX3A, Del, G_8, AAPPRSRPPPRSRPRRRRPRRPRPPRRRPPQRRRPSPPPPPKGRATPSSALSTKRPSCA, 59, 6.7, % positive cancer, 10.7, % positive normal, (SEQ ID NO: 6018); MSH3, Del, A_8, DKKKRATFLLALWECSLPQARLCLIVSRTLLLVQS, 35, 14.6, % positive cancer, 9.7, % positive normal, (SEQ ID NO: 6019); NRXN2, Del, G_8, PGEEEPAMCTSQQKARRSLWRPSKAMSSSATTCHTTPSRAALMRSHWPSAPCNATA, 56, 18, % positive cancer, 12.6, % positive normal, (SEQ ID NO: 6020); ODF3L2, Del, C_8, PAPRRILLRYQARASTTARTQTPTASACLPSPCWGGPGPRDPWRRPPALAPTAQSRSP, 58, 13.5, positive cancer, 10.7, % positive normal, (SEQ ID NO: 6021); PIK3C2G, Del, A_8, SEKDIMQLMLISILGRSGALLQHFRISSFLRPSLIYIFLLITQHNLFILCHVLIILSKI, 59, 10.1, % positive cancer, 7.8, % positive normal, (SEQ ID NO: 6022); PRR12, Del, C_8, HPVRPPPPNVRAWVGQQPPMPLGRPLGLEGQGARLIPPVSLKGFWDPRPTGKGLEGGRHRT, 61, 9, % positive cancer, 7.8, % positive normal, (SEQ ID NO: 6023); PRRC2C, Del, A_8, KRKQMMTTMDLDPVYVHQMLLVGEMVVRLLAHLRHLIKMKSSLARMKAQLEHQSKMISSKWW KRG, 65, 2.2, % positive cancer, 7.8, % positive normal, (SEQ ID NO: 6024); RIOK3, Del, A_8, SMEIAKFPFPLKIIEKCILMKTAIALKMRLTGRILVMIPTDQQNRFPLLKRALLEKEKISPPNMMK, 66, 12.4, % positive cancer, 16.5, % positive normal, (SEQ ID NO: 6025); ROB02, Del, C_8, QSSPFLARSWNTMQWNNKKMAMTVIAGAHHCQYKLTYTKVWKMNWKKMMIGSQHLLFEAWLL LLLSPLDSSPLQLLLHPHGKRCNPCCRLTWMS, 94, 18, % positive cancer, 10.7, % positive normal, (SEQ ID NO: 6026); STAU2, Del, A_8, MLQKQCCYNLVIKHPLIFRINLRRQGKTKDGVVQSLGFLNQQIILQKEFFICLLMFIKRWKPAATK, 66, 16.9, % positive cancer, 7.8, % positive normal, (SEQ ID NO: 6027); STK11IP, Del, C_8, LSAVASALWTTDSGSSWMLRCSAMPRRSSSAASRCQWHWQATLGSSCALWLCLTAGCTC, 59, 4.5, % positive cancer, 11.7, % positive normal, (SEQ ID NO: 6028); STPG1, Del, A_8, DSIVKPRDFLIRRMISQDLGSTMLFTSFIRCPTVSHCPRKELACFPQCAPDWTPSFLNTLQRMHTLSH RILFPRETLVIRVPACSSCQAL, 89, 6.7, % positive cancer, 8.7, % positive normal, (SEQ ID NO: 6029); SYCP1, Del, A_8, TRKHKHFYWKHLKFIGNWILKQFLHKLYLEISHQLIMAYPKIKETICGHLPKILYLHHCQRHIQ, 64, 15.7, % positive cancer, 11.7, % positive normal, (SEQ ID NO: 6030); TNK2, Del, C_8, RPPMTTWPRMRMTLRSAPSTAPSWARGSLPGPARARPTTPLCLSRRGRPLPWRTTCSSRPRVGASR PAPHRPQRSSRRYSRSA, 83, 19.1, % positive cancer, 10.7, % positive normal, (SEQ ID NO: 6031); TRIM23, Del, A_8, ILLYWSFWNDCRMGLLVSMELQKNPLGYLERASFVVMKMKLTLPLYIALCVQLICALSVLKLLILQ RH, 68, 6.7, % positive cancer, 9.7, % positive normal, (SEQ ID NO: 6032); UROC1, Del, A_8, RRCSALVTMATWWLFGSAWSTNWTRRGSAWWTWGQIRHPATTRSMAATTLCS SASRRPRASWP PTLLCSRTWSRKA, 76, 7.9, % positive cancer, 12.6, % positive normal, (SEQ ID NO: 6033); USP42, Del, A8, RRRKRRDIQENQRTLLKIQNCTYPGSPAWRLSPSSGEPRVAFLSLVARLWKASDLSVRKRNTYGWK AGMTGVVSLSMARVI, 81, 15.7, % positive cancer, 11.7, % positive normal, (SEQ ID NO: 6034); VASP, Del, C_8, HPQVCPLRGSQLQRTEQGEDHPLHPLSRQHRALVVGELGPQAWPQLLLEPNSGKSASRRRPQGGPQ PPKLRVVEAEVGDSWKR, 83, 18, % positive cancer, 7.8, % positive normal, (SEQ ID NO: 6035); WDR87, Del, A_8, RKSCLCGRHLWHCTMFCGCCSSDIQKTALLGWNSSTSSWTCTNLSPPESRSCFRSC, 56, 6.7, % positive cancer, 4.9, % positive normal, (SEQ ID NO: 6036); ZNF563, Del, U_8, GPVYYVCMKELTLERNRMNVSSVLKPFLFTVPIEDMRECTLGRNRMNVSSVLKPCLIPVPI, 61, 10.1, % positive cancer, 9.7, % positive normal, (SEQ ID NO: 6037); ABCA10, Ins, A_8, KCSIPVMDFRPLAFSILVWTGSGGHSIILLDSLFNTFNLLLHISGIPAFMGTHVCFGGMHNWLCSFSYI PHICAHHLSQVEKK, 84, 6.7, % positive cancer, 6.8, % positive normal, (SEQ ID NO: 6038); ADAMTSL4, Ins, G_8, GWLLGAPGALWTSPPAQHHPVLPAAPLWPLGSWLSLEPVLRAVRPGPEKPAGSLCWEQW, 59, 7.9, % positive cancer, 11.7, % positive normal, (SEQ ID NO: 6039); ASAH2, Ins, U_8, FNHPKPSSHPGLHSCPTLHSHPAFHSHPELHSHSNFSSAFNPRVSSISELQWLPYWCWTS, 60, 11.2, % positive cancer, 6.8, % positive normal, (SEQ ID NO: 6040); C16orf96, Ins, C_8, PVPPLQCLLSCLGPSPDDASSTTFPATSAAAATADSIPKGPPAGPRVHQALKSSTH, 56, 12.4, % positive cancer, 7.8, % positive normal, (SEQ ID NO: 6041); CXXC4, Ins, G_8, GWRRRRRRRRRGRGWWGRQEILLRRRLLLRLLLLGDPPRRRWRRRRRRRQQDQHAPPKRLPEAG ESWLPARAVVANGKY, 79, 13.5, % positive cancer, 15.5, % positive normal, (SEQ ID NO: 6042); GLIS2, Ins, G_8, GHGPWAARPRPASQSGQEPAAALALWGWRTGLACGLPPCWRSWWQGRGGEGAWVGAHQGPGH GGPQDAP, 69, 13.5, % positive cancer, 8.7, % positive normal, (SEQ ID NO: 6043); GRM1, Ins, U_8, FSPDLFGGVPSPQKPRQESVAGRSVVSALGGQNGRRCHHWSPLLSPSPASGRESAREEVWGDQGA VWHPEGGGHVPHVG, 79, 5.6, % positive cancer, 9.7, % positive normal, (SEQ ID NO: 6044); HOXB3, Ins, C_8, PTPISGRHAPGGRLPALSLLAAVPGQRCPTCQEQGAQRQLHEAGSGPRAPVGPAWLTPAQCRTYQC H, 67, 10.1, % positive cancer, 9.7, % positive normal, (SEQ ID NO: 6045); LIG3, Ins, A_8, TKNRGPHRAGRLGRAGR, 17, 1.1, % positive cancer, 2.9, % positive normal, (SEQ ID NO: 6046); LMTK3, Ins, C_8, RGGHGPVNASSAPDTSPPRHPRRWVSQQRQRLWRQFRVGGGFPPPPPSRPPAVLLPLLRLACAGDP GATRPGPRRPARRPRGEL, 84, 13.5, % positive cancer, 8.7, % positive normal, (SEQ ID NO: 6047); LOXL3, Ins, G_8, GPCSGELCARPCLRGIQWPEEATTVEASGGGPCPSKGRRPPWRGPGRSPEGQHMGHSL, 58, 5.6, % positive cancer, 7.8, % positive normal, (SEQ ID NO: 6048); MEX3A, Ins, G_8, GRRPRAAGRPPAAGPAAAARGAPGRPDGGPRSADAPAPHRPQRGERRQALRSLQRGRAAPEGQQQ HHGVCSRAHLRARGRDRGQARLQD, 89, 9, % positive cancer, 6.8, % positive normal, (SEQ ID NO: 6049); MSH3, Ins, A_8, DKKKGQHFYWHCGSAACHRRGCV, 23, 4.5, % positive cancer, 5.8, % positive normal, (SEQ ID NO: 6050); MYRF, Ins, C_8, QHPQCPDAEWNDQTGAWDRDSPASAPHSSPIATLASPGSALPGPWFLASQHCPCPDTALAPARCPL PRPPAGQ, 73, 6.7, % positive cancer, 8.7, % positive normal, (SEQ ID NO: 6051); NAV1, Ins, A_8, KELASKFLQQSVQYKKGAQVSFLILGYRGDCYTRLFSPLIPQTTAWFYRDCFTLHQVLHLVLRGH, 65, 15.7, % positive cancer, 7.8, % positive normal, (SEQ ID NO: 6052); OR6P1, Ins, U_8, CNLPSDIVGECTYCLHNMACSKPSSSHVLFPWPSLFPGAMVHQCHHSSALGSLSYPGW, 58, 10.1, positive cancer, 9.7, % positive normal, (SEQ ID NO: 6053); PLEKHM2, Ins, G_8, GAVRWLPESQHHGSAPRLPGHSLRPALPGAKCRERGRDGRVDAASLPGCVQRGHPPGRSSQPLHT LLPGPHG, 72, 5.6, % positive cancer, 7.8, % positive normal, (SEQ ID NO: 6054); PLXNB3, Ins, G_8, GVPLAPGESHRGARRGQGAVQQPAGARASKGQGHSGNLPHPSAVHEGHAAEVCGRHLPGHSQRE PAHPHRRQVPV, 75, 11.2, % positive cancer, 10.7, % positive normal, (SEQ ID NO: 6055); POU3F2, Ins, G8, GRGRRRGRRRRLPVVHQPPGPAGHQALGGGAAGRPRRRAARARRPAAAASAAATATAAATAATA AAAAATAAAASGAPRR, 80, 21.3, % positive cancer, 12.6, % positive normal, (SEQ ID NO: 6056); SLC25A24, Ins, A_8, IRTMVEAAFGRRHCWCCLSNKHCPFGPSENHDAGSRFKIRQNEHIWWLSTDGKRRRYPLALEGKW YKRHQNCS, 73, 5.6, % positive cancer, 8.7, % positive normal, (SEQ ID NO: 6057); UBE2Q1, Ins, G_8, GPGAGALPEARAEAARVHLPPRPRALPHCQRLPGRAELRVPAGWGRRGRGGGRARTASPPTGVGA WGSCPHPLQHHGVIPCCAPHLVGGV, 90, 9, % positive cancer, 8.7, % positive normal, (SEQ ID NO: 6058); USP35, Ins, C_8, HQPVHRRPGLQGSWWAEQSGGKDREGGRREGGENGEGRSGGGGGKHQRGRREGERGGGGRGRR EGGEGDRKGG, 73, 5.6, % positive cancer, 9.7, % positive normal, (SEQ ID NO: 6059); VASP, Ins, C_8, PTPRFAPFGGPSCSARSRGRTTPCTPSPGSTGPWWWGSWGPRPGRSYCWSQTQESQQAGGGLRGA HSPQS, 70, 10.1, % positive cancer, 15.5, % positive normal, (SEQ ID NO: 6060); ZFHX4, Ins, C_8, PTSSTFCSSTGPTAGFSGPAALSFHYDATCATPCASSPACPAAATDGRTLCRPHPTLPAAARIRSQLL KTFSVQTPTDKNYR, 82, 12.4, % positive cancer, 8.7, % positive normal, (SEQ ID NO: 6061); ZNF469, Ins, G_8, AAALRVPRRAEAGSAGTEGPGPRGHTAQQTAQGGHARQCPWARRGQASSPGKQPHPE, 57, 14.6, % positive cancer, 9.7, % positive normal, (SEQ ID NO: 6062); ABCA9, Del, A_9, KLIPSYGFQASTLLHTGLAKHWWMFPCTF, 29, 2.2, % positive cancer, 3.9, % positive normal, (SEQ ID NO: 6063); ANKRD49, Del, A_9, WKKTQADCFFGLLKKIGLPQCGDSFLKRPLT, 31, 6.7, % positive cancer, 3.9, % positive normal, (SEQ ID NO: 6064); C15orf48, Del, A_9, IQNLGKLWTLLYLKSL, 16, 1.1, % positive cancer, 2.9, % positive normal, (SEQ ID NO: 6065); CAMSAP1, Del, A_9, PGLRPSQHLLLYITLRVVKWIPALATASAWPAPSAKTVWHPTLLI, 45, 3.4, % positive cancer, 5.8, % positive normal, (SEQ ID NO: 6066); CCDC63, Del, A_9, SQTKNRFSQKCRRPITPGNCRNRFTFWKPV, 30, 7.9, % positive cancer, 5.8, % positive normal, (SEQ ID NO: 6067); CCDC73, Del, A_9, ILFHVRNIVYGIQVMLC, 17, 6.7, % positive cancer, 1, % positive normal, (SEQ ID NO: 6068); CD3G, Del, A_9, NGIWEVMPRTLEGCISVKDHRTSQNHSKCITECVRTALN, 39, 12.4, % positive cancer, 9.7, % positive normal, (SEQ ID NO: 6069); CEL, Del, C_9, PCRPRVTPAPPPCRPRVTPGPPP, 23, 6.7, % positive cancer, 3.9, % positive normal, (SEQ ID NO: 6070); DENND1C, Del, C_9, LPGGIASRFPASWPRTPAACHPSLRTGT, 28, 3.4, % positive cancer, 5.8, % positive normal, (SEQ ID NO: 6071); DIAPH3, Del, A_9, LTDFFVLWKASGTIQFNCK, 19, 3.4, % positive cancer, 4.9, % positive normal, (SEQ ID NO: 6072); DYX1C1, Del, A_9, FREKRNYVKKKSKLKKKEKK, 20, 5.6, % positive cancer, 6.8, % positive normal, (SEQ ID NO: 6073); ELAVL3, Del, G_9, ARPARPCPTGHSLVQMEPLTTARPTSSSTTCPRT, 34, 6.7, % positive cancer, 4.9, % positive normal, (SEQ ID NO: 6074); FBXL3, Del, U_9, YMKKNLTPSFAMKYLPPICTLGDQ, 24, 3.4, % positive cancer, 2.9, % positive normal, (SEQ ID NO: 6075); FLT3LG, Del, C_9, PAVFASSRPTSPASCRRPPSSWWR, 24, 2.2, % positive cancer, 4.9, % positive normal, (SEQ ID NO: 6076); GRB14, Del, A_9, NMEHRLTMDSALSLTKREGPET, 22, 3.4, % positive cancer, 2.9, % positive normal, (SEQ ID NO: 6077); KCNMA1, Del, A_9, SNGMEACGTHPTPRLS, 16, 3.4, % positive cancer, 5.8, % positive normal, (SEQ ID NO: 6078); KCTD16, Del, A_9, KLLKKSSQLRRSWRNVSRIS, 20, 11.2, % positive cancer, 5.8, % positive normal, (SEQ ID NO: 6079); LRRIQ1, Del, A_9, FQKNGALRIFLLLSKC, 16, 6.7, % positive cancer, 5.8, % positive normal, (SEQ ID NO: 6080); LRRIQ3, Del, A_9, NSRKKLLEDMKQNGFT, 16, 9, % positive cancer, 3.9, % positive normal, (SEQ ID NO: 6081); MCPH1, Del, A_9, NVNVCSPKILILKHQKMIRDFRRNLRKWLKSYKGKKQI, 38, 12.4, % positive cancer, 7.8, % positive normal, (SEQ ID NO: 6082); MLH3, Del, A_9, KNLAIVEEEMFLVMGELNYVPLAL, 24, 7.9, % positive cancer, 4.9, % positive normal, (SEQ ID NO: 6083); PEX1, Del, A_9, LGQIIMKKMRRPVCYK, 16, 2.2, % positive cancer, 3.9, % positive normal, (SEQ ID NO: 6085); RAPGEF6, Del, A_9, VIAILSSMCQEILNRHHRRKEVRKLKQILFQVEETKSGRFWIKHDLVSCLQSYLVMEA, 58, 14.6, % positive cancer, 8.7, % positive normal, (SEQ ID NO: 6086); RGS12, Del, A_9, NIRKLIWTKQRSFLSLFPKLRATEQMTNVGC, 31, 3.4, % positive cancer, 4.9, % positive normal, (SEQ ID NO: 6087); RGS3, Del, U_9, FRSPSWSLPFPGSSCLSPSPGLCLGVQSSGQDSRERVWLQPAPLLPAAQDAGWGTGARVVPPTDQS AAPRWGALQRCSEACTSLATGTCRGDTR, 94, 14.6, % positive cancer, 11.7, % positive normal, (SEQ ID NO: 6088); RMDN3, Del, A_9, LLQPCLKALSVPLWKMPSRAS, 21, 1.1, % positive cancer, 5.8, % positive normal, (SEQ ID NO: 6089); RTKN2, Del, A_9, LKRQMGSSLLVSMKNPYHLLGPHSLMVTIKWSSRKRYCILQVSHYMMKKGKRDKLPFLLLINFHS A, 66, 13.5, % positive cancer, 11.7, % positive normal, (SEQ ID NO: 6090); SFRPS, Del, G_9, ACGRPRWRCCWGRCTGRRRAARSTTTMAGRPSRCTAAPTPSRRSALTSLPTCRSATRWATSACGC PTCWSTRAWPK, 76, 14.6, % positive cancer, 9.7, % positive normal, (SEQ ID NO: 6091); SGO1, Del, A_9, CTNLSVPMMLTILIWKRVFILLLSDKK, 27, 2.2, % positive cancer, 2.9, % positive normal, (SEQ ID NO: 6092); SLAMF1, Del, A_9, ALRSMPKSRNQVLFRRNLTPSQLRTLAPPYMLLPQSLSQSLSRKQIPSQSMLV, 53, 11.2, % positive cancer, 10.7, % positive normal, (SEQ ID NO: 6093); SLC35G2, Del, A_9, KGELSLEPWIPYLHQQKTQ, 19, 5.6, % positive cancer, 4.9, % positive normal, (SEQ ID NO: 6094); SYCP2, Del, A_9, TIKISQIQNQSVNKNFHIHLKRTYQ, 25, 5.6, % positive cancer, 6.8, % positive normal, (SEQ ID NO: 6095); TBC1D23, Del, A_9, NILNSLPSSMEIAVLQE, 17, 1.1, % positive cancer, 2.9, % positive normal, (SEQ ID NO: 6096); THAPS, Del, A_9, NPRRKTWKMRKKYAQKPSQKNHLY, 24, 4.5, % positive cancer, 4.9, % positive normal, (SEQ ID NO: 6097); WISP3, Del, A_9, NVLCKQQNGLPAPEHVGWEYLTG, 23, 4.5, % positive cancer, 3.9, % positive normal, (SEQ ID NO: 6098); ABCA9, Ins, A_9, KSSFPATDFRPLPFCILVWPSTGGCFPVLFDPPANANNGLYF, 42, 10.1, % positive cancer, 4.9, % positive normal, (SEQ ID NO: 6099); ABCC4, Ins, A_9, NPGEICPLHRANHCTQIEHHY, 21, 5.6, % positive cancer, 6.8, % positive normal, (SEQ ID NO: 6100); ADAMTS17, Ins, C_9, PWAWRHTLPGCQCRTCGLREPALPQGSAQLPGPAVPGTRPAEPQEERPADSRGG, 54, 11.2, % positive cancer, 11.7, % positive normal, (SEQ ID NO: 6101); ADAMTS18, Ins, U_9, FCNVRQSERWNSLLPKQK, 18, 4.5, % positive cancer, 4.9, % positive normal, (SEQ ID NO: 6102); AK9, Ins, A_9, KRRRRSKKSHRRGIETRRRKSKATGTYESEGKRS, 34, 13.5, % positive cancer, 9.7, % positive normal, (SEQ ID NO: 6103); ATAD2, Ins, A_9, KRRAQRRQSDSSYSVIEG, 18, 5.6, % positive cancer, 3.9, % positive normal, (SEQ ID NO: 6104); ATG2B, Ins, A_9, KIRLSEQELSEFSLSSSEY, 19, 1.1, % positive cancer, 3.9, % positive normal, (SEQ ID NO: 6105); C15orf48, Ins, A_9, KSRTLGNCGPYCTSKAYNNQPTMETH, 26, 1.1, % positive cancer, 2.9, % positive normal, (SEQ ID NO: 6106); CYHR1, Ins, G_9, GDAGCGRPPEPAGGGQAAAATEGGCLSLGLGCRQLGLLPGPAYTAPPVGV, 50, 6.7, % positive cancer, 7.8, % positive normal, (SEQ ID NO: 6109); ELAVL3, Ins, G_9, GPGRPGPAQRATPWYKWSH, 19, 2.2, % positive cancer, 4.9, % positive normal, (SEQ ID NO: 6110); ESCO2, Ins, A_9, NFCDKASAKVLGPRREIENWTTECKQ, 26, 5.6, % positive cancer, 4.9, % positive normal, (SEQ ID NO: 6111); F8, Ins, A_9, NSGRNRKEGNINPRECSFASDTYSDWH, 27, 4.5, % positive cancer, 2.9, % positive normal, (SEQ ID NO: 6112); FGFBP1, Ins, A_9, KSEEWTSQQSGLRTKGHSGQHPD, 23, 5.6, % positive cancer, 3.9, % positive normal, (SEQ ID NO: 6113); HERCS, Ins, A_9, NNSDHMWRLPFSCTLKRW, 18, 2.2, % positive cancer, 4.9, % positive normal, (SEQ ID NO: 6114); IL1R2, Ins, A_9, KRRDHSCDHFPPQDHISFSGVKTDNPV, 27, 2.2, % positive cancer, 4.9, % positive normal, (SEQ ID NO: 6115); INO80E, Ins, A_9, KIPESEAEAQVPHLRARVLPGGAEESAKEITEGVPGQEFPPRPTSAVRERG, 51, 9, % positive cancer, 5.8, % positive normal, (SEQ ID NO: 6116); MCHR2, Ins, U_9, FSPTLDFGVLYFNFMLYLGDVSTE, 24, 2.2, % positive cancer, 6.8, % positive normal, (SEQ ID NO: 6117); MUC15, Ins, A_9, KTNTKEAYNVGLSQNSVDFNVVLFTSIGEPWKRKSRHKHNTEHCRSF, 47, 5.6, % positive cancer, 10.7, % positive normal, (SEQ ID NO: 6118); MYO6, Ins, A_9, KTAGRGSRKAEAYSRRNGKGKKKT, 24, 6.7, % positive cancer, 3.9, % positive normal, (SEQ ID NO: 6119); PADI4, Ins, A_9, KTAENKEHSVKQDIERT, 17, 10.1, % positive cancer, 4.9, % positive normal, (SEQ ID NO: 6120); PRR11, Ins, A_9, KRSLSLSVQANYTSSSTTLTRKSRYFFNRYISKQKLANIILELQFS, 46, 7.9, % positive cancer, 5.8, % positive normal, (SEQ ID NO: 6121); RBL1, Ins, A_9, KVICTFYPTDRTEIFTRKRSSHYSCCISHPKCEPVTEYCGWSEKCTK, 47, 12.4, % positive cancer, 6.8, % positive normal, (SEQ ID NO: 6122); RBM27, Ins, A_9, KTGSNEVTTRYEEKKTGSVRKANRMPKDVNIQVRKKQKHETRRKSKYNEDFERAWREDLTIKR, 63, 15.7, % positive cancer, 7.8, % positive normal, (SEQ ID NO: 6123); RGS22, Ins, A_9, KIGSPRRRKIWKGWNQTICKYFSACYQNCFTQ, 32, 3.4, % positive cancer, 2.9, % positive normal, (SEQ ID NO: 6124); ROM1, Ins, G_9, GAPGRRPRASPGFAWESG, 18, 2.2, % positive cancer, 2.9, % positive normal, (SEQ ID NO: 6125); SACS, Ins, A_9, KILLFQSIEHTCLKCFKRGC, 20, 5.6, % positive cancer, 2.9, % positive normal, (SEQ ID NO: 6126); SFRPS, Ins, G_9, GRADGRAGAAAGGAALGAGALRGVRLLWLAGRAAARPLLLQAAAVP, 46, 6.7, % positive cancer, 4.9, % positive normal, (SEQ ID NO: 6127); SLAMF1, Ins, A_9, KPYDLCPSPETRSSSEET, 18, 2.2, % positive cancer, 4.9, % positive normal, (SEQ ID NO: 6128); SLC35G2, Ins, A_9, KRESFLWNHGYPTSTNRRPNDQ, 22, 9, % positive cancer, 3.9, % positive normal, (SEQ ID NO: 6129); SPOUT1, Ins, A_9, KMEGSQADEKTGAAAGTGGTGKAPGRGGGSGREGGPRAALHTERSPAGLHPGQCSVAGASHLLG RSDCQSLCHLLCG, 77, 16.9, % positive cancer, 7.8, % positive normal, (SEQ ID NO: 6130); SYCP2, Ins, A_9, NERKVKRERIYQCSRILDKPNQ, 22, 2.2, % positive cancer, 3.9, % positive normal, (SEQ ID NO: 6131); SYCP3, Ins, A_9, NYDGNSAARDSKCSEVSSIHVIL, 23, 5.6, % positive cancer, 3.9, % positive normal, (SEQ ID NO: 6132); TAS2R50, Ins, U_9, FNSNNGFICSRKLCQWLHSTGKFH, 24, 5.6, % positive cancer, 2.9, % positive normal, (SEQ ID NO: 6133); TMEM196, Ins, A_9, KIRTCHDPLFSLLYLWTYWGHPEFSVPPGSHKENFLPIPTAPCLHVSRVHWDRGLHSLFLAHLSTSQ L, 68, 14.6, % positive cancer, 11.7, % positive normal, (SEQ ID NO: 6134); USP40, Ins, A_9, KTRLFARGTVLLERRRYYWC, 20, 6.7, % positive cancer, 5.8, % positive normal, (SEQ ID NO: 6135); VEPH1, Ins, A_9, KPQQSESICHGNWKEDSSP, 19, 5.6, % positive cancer, 4.9, % positive normal, (SEQ ID NO: 6136); VPS37B, Ins, C_9, PTTPGACGTLSHPVYCGHEFGTGRAVPRITVPAPAPPRGPPHSARILFAVRVPISATSPSETPAPAPST PAGFHPPV, 77, 19.1, % positive cancer, 9.7, % positive normal, (SEQ ID NO: 6137); WISP3, Ins, A_9, KMSCASNKMDSLLQNMWDGNI, 21, 5.6, % positive cancer, 4.9, % positive normal, (SEQ ID NO: 6138); CASPS_lstms, Del, A_10, HKKKQLRCWNTWAKMFFMVFLIIWQNTMF, 29, 12.4, % positive cancer, 11.7, % positive normal, (SEQ ID NO: 6139); CASP5_2 ndms, Del, A_10, KGVRILKLCSKVSFRVDWITS, 21, 6.7, % positive cancer, 3.9, % positive normal, (SEQ ID NO: 6140); CEP290, Del, A_10, KFVKWLKKEEKEVQLQD, 17, 9, % positive cancer, 5.8, % positive normal, (SEQ ID NO: 6141); EFHC2, Del, A_10, MYYPPKTLKGCANPPDYL, 18, 7.9, % positive cancer, 4.9, % positive normal, (SEQ ID NO: 6142); FAM111B, Del, A_10, KHYNRKISIKKLNRMKVPLMKLITRV, 26, 2.2, % positive cancer, 3.9, % positive normal, (SEQ ID NO: 6143); G2E3, Del, A_10, KLKDCISTKPISGIVP, 16, 3.4, % positive cancer, 3.9, % positive normal, (SEQ ID NO: 6144); HMGXB4, Del, A_10, RKKKRRTKREREEKSQKRRTCRPTRCSVKSIA, 32, 5.6, % positive cancer, 7.8, % positive normal, (SEQ ID NO: 6145); KNOP1, Del, A_10, KSTRREMPSQATPSPPGPWRAALGKEVKRSQSKLRLRNTSP, 41, 13.5, % positive cancer, 7.8, % positive normal, (SEQ ID NO: 6146); LRRIQ1, Del, A_10, IRHTDTQQDLQVSCGFLQN, 19, 1.1, % positive cancer, 1.9, % positive normal, (SEQ ID NO: 6147); OR52N5, Del, U_10, LAMLSPSLTSLPAPPLYPMHSASSGSVSKKLTSMLAWPRCSLFMGSQVWSLGCSCSWL, 58, 4.5, % positive cancer, 7.8, % positive normal, (SEQ ID NO: 6148); SMAP1, Del, A_10, RKRKREKRSQKSRQNHLQLKSCRRKISNWSLKKVPALKKLRSPLWIF, 47, 6.7, % positive cancer, 4.9, % positive normal, (SEQ ID NO: 6149); SPINK5, Del, A_10, RKRMKTGAIQEKGAIQEKGAMTKRICVVNFEACREMESLSAPEKITLFEAHMARCTSINVLCVRAS LIEKLMKEKRKMKRNQVASPQIMQRMSAVNFETI, 100, 9, % positive cancer, 8.7, % positive normal, (SEQ ID NO: 6150); SREK1IP1, Del, A_10, KGKGLTHPVPLKRTLQNKRNKNIRRKKRKKKKRVNQKKGNITKRKKRREKRKSILLHLIVLNSPES, 66, 12.4, % positive cancer, 12.6, % positive normal, (SEQ ID NO: 6151); ULK4, Del, A_10, RTLESAGLFPWLHTQC, 16, 3.4, % positive cancer, 4.9, % positive normal, (SEQ ID NO: 6152); ZNF512B, Del, C_10, LRSPQTSPLRLRTRWVWSGPQAGVSAARRPRWRCSTCRR, 39, 4.5, % positive cancer, 4.9, % positive normal, (SEQ ID NO: 6153); AKNAD1, Ins, A_10, KTSGFHDLQLRPCNTLTPFLLLQDFWKQILM, 31, 3.4, % positive cancer, 4.9, % positive normal, (SEQ ID NO: 6155); CCDC148, Ins, A_10, KNKKILGQEKTEVARNGNERSSASRRTEEINR, 32, 2.2, % positive cancer, 1.9, % positive normal, (SEQ ID NO: 6156); CEP290, Ins, A_10, KNSSNGSRKRKKKCNFRINH, 20, 9, % positive cancer, 4.9, % positive normal, (SEQ ID NO: 6157); HMGXB4, Ins, A_10, KEKRREGQRERERRKAKKEEHVGLPGVL, 28, 3.4, % positive cancer, 4.9, % positive normal, (SEQ ID NO: 6158); ILS, Ins, A_10, KVWRRKTESKPIPRLPARVSWCNEHRVDNRKL, 32, 3.4, % positive cancer, 4.9, % positive normal, (SEQ ID NO: 6159); ISL1, Ins, A_10, KTSDFPMCWLRQSDSRSVYSEGFSGFGMACGMFEMCGV, 38, 10.1, % positive cancer, 7.8, % positive normal, (SEQ ID NO: 6160); KNOP1, Ins, A_10, KNPPGGRCPPRPLQALQVHGEQP, 23, 2.2, % positive cancer, 4.9, % positive normal, (SEQ ID NO: 6161); OR7C1, Ins, U_10, FHFIWMPGQFTLDRDGL, 17, 4.5, % positive cancer, 1.9, % positive normal, (SEQ ID NO: 6162); RBM43, Ins, A_10, KNPEFKLQSFETQWKNLRGRIISGCQEAQRIFASKSMFSLRKRQKFYQ, 48, 6.7, % positive cancer, 5.8, % positive normal, (SEQ ID NO: 6163); RXFP2, Ins, A_10, KKFIYIHCVDRGLLFPETWGFEQNNTWRQYNETSFL, 36, 13.5, % positive cancer, 5.8, % positive normal, (SEQ ID NO: 6164); SMAP1, Ins, A_10, KGREKERKGARKAGKTTYS, 19, 1.1, % positive cancer, 4.9, % positive normal, (SEQ ID NO: 6165); ASTE1, Del, A_11, SKKKGRRNRIPAVLRTEGEPLHTPSVGMRETTGLGC, 36, 12.4, % positive cancer, 11.7, % positive normal, (SEQ ID NO: 6166); CCDC150, Del, A_11, QKKKKSWRLLLHSSNLI, 17, 3.4, % positive cancer, 1.9, % positive normal, (SEQ ID NO: 6167); CEP164, Del, A_11, KKKKRKRKTRRTETPPKVRWPWVPH, 25, 12.4, % positive cancer, 9.7, % positive normal, (SEQ ID NO: 6168); LTN1, Del, A_11, SKKKMVRLDLLMRYLKAIKRMKNVYLQKERRLKAGN, 36, 13.5, % positive cancer, 5.8, % positive normal, (SEQ ID NO: 6170); SLC22A9, Del, A_11, AQKKNLLCVKCSTCPTYVKGSPSCPLRDLQTLWPILALISMSSIWGTMFSCCRLSLVQSSSWPTVLH LGH, 70, 9, % positive cancer, 10.7, % positive normal, (SEQ ID NO: 6171); KIAA0146, Ins, U_11, CHFFLFFFSCPSSRTTTFDS, 20, 1.1, % positive cancer, 4.9, % positive normal, (SEQ ID NO: 6172); LOC100287534, Ins, A_11, SKKKCCCNEPRACGTQNSEQGGL, 23, 4.5, % positive cancer, 8.7, % positive normal, (SEQ ID NO: 6173); OR7E24, Ins, U_11, LFFFPQKVSELHRATESHRCLRIPPPGTLRGSRTAAGPRWAVPVHVPGHGAGEPAHHPGCQL, 62, 10.1, % positive cancer, 9.7, % positive normal, (SEQ ID NO: 6174); CHST15, Del, C_12, TPPPVPPELAPGRRSWFVVIMQAALWVCVSA, 31, 6.7, % positive cancer, 6.8, % positive normal, (SEQ ID NO: 6175); USP4, Del, U_13, FLRWSLTLSPSWSAVVQSRLTPGFK, 25, 2.2, % positive cancer, 1.9, % positive normal, (SEQ ID NO: 6176); LOC100130156, Del, U_14, VFFFLRESHSVAQAGVQWHHLSSLQPPPPGFKRFSCLSSRVA, 42, 16.9, % positive cancer, 8.7, % positive normal, (SEQ ID NO: 6177); C15orf40, Ins, U_14, FFFFETKSCSVAQAGVQWRSLGSLQPPPPGFKLFSCLSFL, 40, 12.4, % positive cancer, 10.7, % positive normal, (SEQ ID NO: 6178); LOC100652894, Del, U_15, FFFFLTRSLALSPRLERSGAISAHYNLRLLS, 31, 6.7, % positive cancer, 4.9, % positive normal, (SEQ ID NO: 6179); and LOC100652963, Ins, U_18, FFFFETGVLLCRPGWSAVGQFGLLQPPSPGFKQFSCLSHPSSWDYRHPPPYLANL, 55, 9, % positive cancer, 13.6, % positive normal, (SEQ ID NO: 6180).

The following are the total dog MS antigens on the array and their positive rate in a total 349 samples from 9 different cancers compared to 242 healthy dogs. The cutoff for the positive of each peptide is the average signal plus 2 fold standard deviations of the healthy dogs. USP8, Del, A_7, KMRPQRKGQSQQRSCIQ, 17, essential, driver, high expression, 2.29, % positive cancer, 2.07, % positive normal, SEQ ID NO: 6181); C1S, Del, A_7, KRAGNFVTVEIQSLVPKKSLPILFGSLRKQYMYSKM, 36, high expression, 8.31, % positive cancer, 4.96, % positive normal, SEQ ID NO: 6182); PLIN2, Del, A_7, KKLKGLIQFRNQVITLDWAPCLPSSAHVPTNRLSAGLKKPSKKAKRPFLSSIPLLI, 56, high expression, 6.59, % positive cancer, 7.02, % positive normal, SEQ ID NO: 6183); C1S, Ins, A_7, KKGLETSLPWRSNPLFQRSHCQFCLGA, 27, high expression, 2.58, % positive cancer, 4.13, % positive normal, SEQ ID NO: 6184); CAPZA2, Ins, A_7, KKNRWTANHYCMHREPSVPSKKLLEWSLEVRVEVYNHSFNHSSGWHLENSGSLL, 54, high expression, 6.88, % positive cancer, 6.61, % positive normal, SEQ ID NO: 6185); TMPO, Ins, A_7, KKRTLHSHVDKNFAVCCCGHFFVFGLSSYGNQPRKSVQ, 38, high expression, 11.17, % positive cancer, 7.02, % positive normal, SEQ ID NO: 6186); ZAK, Ins, A_7, KKREINHSKQLPQKFC, 16, high expression, 7.16, % positive cancer, 5.79, % positive normal, SEQ ID NO: 6187); NSRP1, Del, A_7, KKERKIILNCFWEKTESPSIFTTY, 24, essential, 6.88, % positive cancer, 2.07, % positive normal, SEQ ID NO: 6188); EIF2B5, Ins, A_7, KKCICHDNDLQRVIP, 15, essential, 3.15, % positive cancer, 3.31, % positive normal, SEQ ID NO: 6189); PCBP2, Ins, C_7, PPEGRDHPVPAQAVQFSSHLCRWSGLYHSRTVCHSTARFDQAAPVGNATVSFSHDAWQHRIQCRF GCICSDYFS, 74, essential, high expression, 7.45, % positive cancer, 8.68, % positive normal, SEQ ID NO: 6190); CCDC80, Del, G_7, GATAALPRPRPGRPKSTRSSRSSRSSRSPRRRRGRS, 36, high expression, 5.44, % positive cancer, 3.31, % positive normal, SEQ ID NO: 6192); NUTM1, Del, G_7, GGAASAGTSPPRGPHSPASGGGTLLSAAEGRSAAVVS, 37, driver, 10.32, % positive cancer, 6.61, % positive normal, SEQ ID NO: 6193); NUTM1, Ins, G_7, GGARRLRGQAPRAGPTAPQAAAGHFCQRPKEEAPP, 35, driver, 5.44, % positive cancer, 3.72, % positive normal, SEQ ID NO: 6194); TNPO1, Del, U_7, FFVMLLHRGLTQKMI, 15, essential, high expression, 4.58, % positive cancer, 4.13, % positive normal, SEQ ID NO: 6195); XRCC6, Del, U_7, FWLMVLGPCLNLRVKLN, 17, essential, high expression, 1.72, % positive cancer, 1.24, % positive normal, SEQ ID NO: 6196); PPP1CC, Del, U_7, FFFSEETMNVSASIEFMDFTMNVKEDITLNYGKLSQTALTVYR, 43, high expression, 4.87, % positive cancer, 6.2, % positive normal, SEQ ID NO: 6197); MFN2, Ins, U_7, FFWPDEQREEHCNQCHALGQSSALRDRPHHQLLPAGRRHRWS, 42, essential, 6.02, % positive cancer, 5.79, % positive normal, SEQ ID NO: 6198); PRPF4B, Del, A_8, KKRNINTEVNIRNINIPQKKTRIKNINISINIRNTKEKKLLMLLTKKVCLQLKELNLMI, 59, essential, high expression, 8.6, % positive cancer, 6.2, % positive normal, SEQ ID NO: 6199); DDX24, Ins, A_8, KKKERKKKIGAFPGYCSKGAQKSKDMDA, 28, essential, high expression, 1.15, % positive cancer, 1.24, % positive normal, SEQ ID NO: 6200); SWAP70, Del, A_8, KKISPRVPCSLQKTMHLKYGLFSTFCLRTSIH, 32, high expression, 9.17, % positive cancer, 4.55, % positive normal, SEQ ID NO: 6201); RHOQ, In, A_8, TVKKNRIEMYKLLFDYVKNIFSGQRDCPFLSKSI, 34, high expression, 7.45, % positive cancer, 4.96, % positive normal, SEQ ID NO: 6202); RHOQ, Ins, A_8, KKKNRIEMYKLLFDYVKNIFSGQRDCPFLSKSI, 33, high expression, 6.88, % positive cancer, 5.79, % positive normal, SEQ ID NO: 6203); SWAP70, Ins, A_8, KKKSHQESPAHYRRRCI, 17, high expression, 6.02, % positive cancer, 3.72, % positive normal, SEQ ID NO: 6204); RFC1, Del, A_8, KKTKEKEKLAQLRRNQNLKEVDLLPRRTAL, 30, essential, 8.31, % positive cancer, 3.31, % positive normal, SEQ ID NO: 6205); IRF4, Del, C_8, PPGAQSPRPPRAPGGPAPPPPRHALPRPEPGLPRGAGSPPAAPPRPDPGRAALPAQLRPRLRPSGLLSF ISSSFPPRHLLGFTS, 84, driver, 7.16, % positive cancer, 4.55, % positive normal, SEQ ID NO: 6206); HNRNPH1, Del, U_8, FFLTAKFKMGLKVFVSSTPEKADRVARLLLNLNQKMKSNWP, 41, essential, high expression, 11.46, % positive cancer, 8.68, % positive normal, SEQ ID NO: 6207); DPAGT1, Del, U_8, FFSPPWDCSTITGTHHRCLWEIPSVTLLA, 29, essential, 2.29, % positive cancer, 3.72, % positive normal, SEQ ID NO: 6208); VPS33A, Ins, U_8, FFCQTQARTNGYNC, 14, essential, 2.01, % positive cancer, 0.83, % positive normal, SEQ ID NO: 6209); SEC63, Ins, A_9, KKKEAFKKKTYTCALTTVKATETKAGKWNCWE, 32, essential, high expression, 3.15, % positive cancer, 1.65, % positive normal, SEQ ID NO: 6210); PDS5B, Del, A_9, KKNLDLQHQKKRKKKKDKVETQNRSQRANSTEHQGEHNREQNLLKLVQLNPHNPHHRKDEEDH QKRHHHHNQKKMSV, 77, high expression, 14.9, % positive cancer, 8.68, % positive normal, SEQ ID NO: 6211); PRDM2, Del, A_9, KKKCRLHPRKVDTRHLPAVTKTAAATGAGLQTRRLRCRACRLLWARPERAAPALRQARCRPRPSG PSRMSNLQLR, 75, high expression, 4.87, % positive cancer, 4.96, % positive normal, SEQ ID NO: 6212); SP3, Del, A_9, KKIPGPLRPCPPPLPASSPAAAAFAPHHVTLSRHRSPCWPLPAAR, 45, high expression, 10.32, % positive cancer, 6.61, % positive normal, SEQ ID NO: 6213); SPAG9, Del, A_9, KKGQAFGNFSADFSAPQVIQLRSLNHLLI, 29, high expression, 6.02, % positive cancer, 7.85, % positive normal, SEQ ID NO: 6214); ZCCHC17, Del, A_9, KKNIETGSHLTLTAQTLRVTQARGQGTHQKTARQQRRRRRRRSIRRNTRS, 50, high expression, 4.01, % positive cancer, 2.48, % positive normal, SEQ ID NO: 6215); ARV1, Ins, A_9, KKKTQLHFAAESIIIIQLWKALAHSSCHLGA, 31, high expression, 3, % positive cancer, 4.21, % positive normal, SEQ ID NO: 6216); PDS5B, Ins, A_9, KKKIWTFSTRRRGRRRKTKWKHRTEVKEQTAPNIKESTTESRIS, 44, high expression, 6.88, % positive cancer, 3.31, % positive normal, SEQ ID NO: 6217); SP3, Ins, A_9, KKKSPDRSGRVRRRFPHPLPPPPPSLLTM, 29, high expression, 10.03, % positive cancer, 5.37, % positive normal, SEQ ID NO: 6218); SPAG9, Ins, A_9, KKKVKHLAIFQPTFQLLK, 18, high expression, 6.3, % positive cancer, 3.72, % positive normal, SEQ ID NO: 6219); MLLT10, Del, A_9, KKNIKKRTNTNKNTRSNQNHHLHWFLL, 27, driver, 2.29, % positive cancer, 3.31, % positive normal, SEQ ID NO: 6220); CDC7, Del, A_9, NMSRILNYQVLKKISRSFMKLYHSLVICLRLRTKLEKALLALFIWPQHSYE, 51, essential, 15.76, % positive cancer, 8.26, % positive normal, SEQ ID NO: 6221); GINS1, Del, A_9, KKIASISYPDGSVSSWLDREFWSTCCP, 27, essential, 2.87, % positive cancer, 1.65, % positive normal, SEQ ID NO: 6222); HGS, Del, A_9, KKKSTTRIPMWPCTPWRSWSLW, 22, essential, 6.88, % positive cancer, 1.65, % positive normal, SEQ ID NO: 6223); RFC1, Del, A_9, KKKERVLIKKQSL, 13, essential, 2.01, % positive cancer, 1.24, % positive normal, SEQ ID NO: 6224); TCOF1, Del, A_9, KKKTRKKRKRRRRQKRPQPKTLTHYSRRKRRKRRRQQSKLS, 41, essential, 8.31, % positive cancer, 4.96, % positive normal, SEQ ID NO: 6225); HGS, Ins, A_9, KKKSQRQESPCGLVRPGGHGVCGEELWPDGARRGGQQADHGGA, 43, essential, 5.44, % positive cancer, 2.89, % positive normal, SEQ ID NO: 6226); TCOF1, Ins, A_9, KKKRQGKKGKEEEGKKGLNQRP, 22, essential, 2.58, % positive cancer, 1.24, % positive normal, SEQ ID NO: 6227); SPEN, Del, C_9, PPHHFMHEKDVMSGDLMGLQITGSVLMNIVPMDTMNGGREDLIGQDITIRITIEILASGLYNMGSIIL LGVEVQTVLMLMTPDTSLGLGSSLHCPVWYTGISTGMILPGRYEAEGQSGITSTAGVGHHIHPNLEI SLLRGWLAKRLDPQGPLAAAALGVDPPVVIQSAAAVVPAVTALIPAVVLATTLPPDLFSQQPSQHL LPSCFHPWKKMSHVKVLGSRFRIFQYALQIQALKMAFSMNLRNLER, 247, essential, driver, high expression, 11.46, % positive cancer, 10.33, % positive normal, SEQ ID NO: 6228); PELP1, Ins, C_9, PPPTSSPCAGPCGPAAATVGTRGDSWWRRTPCPGGRPDGY, 40, essential, high expression, 1.72, % positive cancer, 2.89, % positive normal, SEQ ID NO: 6229); CCDC80, Del, C_9, PPPRGPGRAGGRGRGRSTGRGTGRGRGRGRPRAPGTTAPTGGSTAPGAPRAGPRTPSPPRARPPRR KARTEPGATSTRVSMTPAGPAPLSGTPSCGGATAALPRPRPGRPKSTRSSRSSRSSRSPRRRRGRS, 132, high expression, 6.3, % positive cancer, 5.37, % positive normal, SEQ ID NO: 6230); MAP4K4, Del, C_9, PPRARCAPPAGRPLFRILLGFLSWWKWLEMAPTDKSIRVDMLKQVSWRPSKLWMSLRMKRKKSN WR, 66, high expression, 6.59, % positive cancer, 6.61, % positive normal, SEQ ID NO: 6231); RHOBTB3, Del, C_9, PPPARGAPAAPRRAPAAHHPPTPGGSRPSTSWRWGTRGTRFTRTAGRRGSSALTWGEARERGG, 63, high expression, 7.16, % positive cancer, 11.16, % positive normal, SEQ ID NO: 6232); CCDC80, Ins, C_9, PPPHGARAGQGAGAGAGARAGARAGAGAGAGRARPGQPHRPAGARPPGPRGRARAPQARQGQG RPEERPGQNREQRVRE, 79, high expression, 9.17, % positive cancer, 7.02, % positive normal, SEQ ID NO: 6233); MAP4K4, Ins, C_9, PPPARGARRPPAGHCSGSCWDF, 22, high expression, 4.58, % positive cancer, 2.89, % positive normal, SEQ ID NO: 6234); RHOBTB3, Ins, C_9, PPPRPEGPPLLPAARLLPTTPPRPAGPVHPHRGAGERGGRVSPGQPAVGAHPHLPGEKPANGGDESS F, 68, high expression, 8.6, % positive cancer, 4.96, % positive normal, SEQ ID NO: 6235); VCL, Del, G_9, GGVFPGGGAKPNFALRRKSQNHIRVLLSLPCP, 32, essential, high expression, 3.15, % positive cancer, 1.65, % positive normal, SEQ ID NO: 6236); ABCA1, Del, G_9, GGASRSSPSTGTRTTTTKPSLVATARTKMLAASMTILRLLIAMI, 44, high expression, 3.15, % positive cancer, 2.48, % positive normal, SEQ ID NO: 6237); NUTM1, Del, G_9, GGGRRAREARPGLRLRGGAASAGTSPPRGPHSPASGGGTLLSAAEGRSAAVVS, 53, driver, 4.58, % positive cancer, 2.48, % positive normal, SEQ ID NO: 6238); RECQL4, Del, G_9, GGARTPAVSPRRCRSPAAGGRT, 22, driver, 2.58, % positive cancer, 3.31, % positive normal, SEQ ID NO: 6239); NUTM1, Ins, G_9, GGGDGGRGRPGPGCGCGGARRLRGQAPRAGPTAPQAAAGHFCQRPKEEAPP, 51, driver, 4.87, % positive cancer, 2.48, % positive normal, SEQ ID NO: 6240); RECQL4, Ins, G_9, GGGLGRPPCLPADAGAQLLGAALESGSDAEASRRRLCARLCGGRGRLRGEA, 51, driver, 0.57, % positive cancer, 1.65, % positive normal, SEQ ID NO: 6241); SETD1A, Del, G_9, GGGPALREKKLGPPPAQLHLPAPAPRPQRPPTRACPSLNIAAWIPASRCC, 50, essential, 2.01, % positive cancer, 2.89, % positive normal, SEQ ID NO: 6242); C6, Del, U_9, FFRKKKWNTGVPLTRCPRNTKVPFCREQRNPYP, 33, high expression, 2.29, % positive cancer, 2.48, % positive normal, SEQ ID NO: 6243); SERPINA3, Del, U_9, FFPERSAHWSLFVSTDPFCSAYTTKIRRTSSSGARSPTPIKP, 42, high expression, 7.16, % positive cancer, 5.37, % positive normal, SEQ ID NO: 6244); SERPINA3, Ins, U_9, FFFRKDRPTGHCSFQQTLSVLHTPQRYGEHPLLGQGHQPQSSL, 43, high expression, 7.45, % positive cancer, 6.61, % positive normal, SEQ ID NO: 6245); TGFBR2, Del, A_10, KEKRRCWGRLSLCVPVAPTSATTTSSSLKNMPPTTLTCC, 39, driver, high expression, 9.17, % positive cancer, 8.26, % positive normal, SEQ ID NO: 6246); SEC63, Del, A_10, KKNLHLCPYHSQSNRNKSRQMELLGVKLR, 29, essential, high expression, 1.43, % positive cancer, 1.24, % positive normal, SEQ ID NO: 6247); ANKRD12, Del, A_10, KKTKQQTVVTTLSQKKVKIKKRTGK, 25, high expression, 4.01, % positive cancer, 2.89, % positive normal, SEQ ID NO: 6248); HK1, Del, A_10, KKKSRTRNYQWDSHFLSLVGSPR, 23, high expression, 1.43, % positive cancer, 2.48, % positive normal, SEQ ID NO: 6249); CHD2, Ins, A_10, ILLNLSSESCTF, 12, high expression, 0.43, % positive cancer, 2.63, % positive normal, SEQ ID NO: 6250); HK1, Ins, A_10, KKKNQGQEITSGIHIFFPLSAVQDRRGHPDHLDKEI, 36, high expression, 4.01, % positive cancer, 2.89, % positive normal, SEQ ID NO: 6251); EIF2B3, Del, A_10, KKKQWSSVTSLEWTAQERGCSSWLMKRTWMKSWSLRDPSCRSTQEYISTQALWTPISTA, 59, essential, 8.88, % positive cancer, 10.74, % positive normal, SEQ ID NO: 6252); POP4, Del, C_10, PPPASTEPRGAAWPECAGPAGSPHGPSCVWICADGVPPTVRFPRKRRRSWRFSFRERSRPRPS, 63, essential, 5.16, % positive cancer, 7.02, % positive normal, SEQ ID NO: 6253); POP4, Ins, C_10, PPPQQARSPGAQPGQSAPGLQEAPTDPRVCGSAQMGCHLPCVFPERGEGAGGSASGSAAGRGLRES LPEAQHAPHEPAGLRGPAATQGCGPGVLHPPQTEGEEKKIQRPVRPAEEGAAPL, 120, essential, 9.17, % positive cancer, 7.02, % positive normal, SEQ ID NO: 6254); SLC35F5, Ins, U_10, LLCVVSGKFVVSRSTFRHTGCYS, 23, high expression, 3.43, % positive cancer, 2.11, % positive normal, SEQ ID NO: 6255); SEC62, Del, A_11, KKKMGKRKSLKRRKLQELPKRRKLRKNSNLSHMMIKFFWMEMRYLCGSMTQFTLKHLSWD, 60, essential, high expression, 12.89, % positive cancer, 6.2, % positive normal, SEQ ID NO: 6256); RUFY1, Del, A_11, KKNPCPPGRRPPPPRAPARAQSSARRGHVTRPRWPKGKTAPWRGGNRGRNPDKISRSWTAASCLA PANCGARGSLRRFRRAGRRPS, 86, high expression, 13.47, % positive cancer, 10.74, % positive normal, SEQ ID NO: 6257); SYTL4, Del, A_11, KKKPPPPEGKEKPASSGTPSIHCMMRPSDMRSQNLFWPRGLCSSQFGIMVVLAETLSLERQRSRWIP GSLMRNWIIASLYMERSVLSPRLA, 91, high expression, 12.03, % positive cancer, 7.02, % positive normal, SEQ ID NO: 6258); RUFY1, Ins, A_11, KKKIPARPDGDRRPPARPPAPSPPPAGAT, 29, high expression, 1.15, % positive cancer, 2.48, % positive normal, SEQ ID NO: 6259); SYTL4, Ins, A_11, KKKNPPPPREKKNQHQAGHHQSTV, 24, high expression, 5.73, % positive cancer, 2.07, % positive normal, SEQ ID NO: 6260); SMARCD1, Del, C_11, PPPRLKGCTAPRCPERPIRDQACCQAAE, 28, essential, driver, high expression, 0.57, % positive cancer, 2.07, % positive normal, SEQ ID NO: 6261); SMARCD1, Ins, C_11, PPPPASRAVPLPDARSGLSETRHAARQPNDTSGTFHGTPWLWGEPFSPTWPGPVRDGPVPQETCTSA DTAGPAAGGPKSKPQCKEKEDG, 89, essential, driver, high expression, 0.86, % positive cancer, 2.48, % positive normal, SEQ ID NO: 6262); RAB12, Del, C_11, PPPRTPCRAALHSGRTGLSCCLRCRDGEPESGVLTRWVAHSAHRRTRALPGRGAWQRPGSIVTLGP KEQAQVVPVLTLKSRL, 82, high expression, 6.88, % positive cancer, 6.61, % positive normal, SEQ ID NO: 6263); RAB12, Ins, C_11, PPPPAPPAGRPCIRGGRASPAASDAEMENQRAGSLPAGWRTALTDGLGPCLAEVPGRGPAVL, 62, high expression, 9.74, % positive cancer, 4.13, % positive normal, SEQ ID NO: 6264); LAMB1, Del, G_11, GGGRSSSRRTKNASSAIHKILITRPSILTVISLKMWSLHLLQTALRFGGNLKMAWKM, 57, high expression, 6.59, % positive cancer, 4.96, % positive normal, SEQ ID NO: 6265); LAMB1, Ins, G_11, GGGGDPVPGGQKMLHLQFTRSLSRDPQS, 28, high expression, 5.44, % positive cancer, 4.13, % positive normal, SEQ ID NO: 6266); DTYMK, Del, G_11, GGGLLSGAPAAQTAGAASATRAAAASGPGEAPAEAASSGSRGFGSRPRPRTKGR, 54, essential, 6.59, % positive cancer, 7.44, % positive normal, SEQ ID NO: 6267); DTYMK, Ins, G_11, GGGGCCPGLPRPKRRGRQVQRGRRPPRDPGRLQPKLRPRGAAGSGPAPAPGRRAVN, 56, essential, 12.61, % positive cancer, 10.33, % positive normal, SEQ ID NO: 6268); ZFAND6, Del, A_12, KKKQKNQTKQNKTRIAVSCAGRKWNLLGLNAGVEVFTVLYTVTPMYTIVLTITKLMLLRKIRKKKI Q, 67, high expression, 6.02, % positive cancer, 3.31, % positive normal, SEQ ID NO: 6269); ZFAND6, Ins, A_12, KKKNKKTKPNKTKQESLFHVQEESGTYWV, 29, high expression, 4.87, % positive cancer, 1.24, % positive normal, SEQ ID NO: 6270); FAM96B, Del, G_13, GGGDARRLRSAVNGAGSTCPGPQPARRRTSRRRRCPGTRSLRPREPAAASAAPPPQGLGRPRTARG ASL, 69, essential, 15.19, % positive cancer, 7.85, % positive normal, SEQ ID NO: 6271); IRF4, Ins, C_15, PPPPPDTRSRGPSPASPAAPGPRQQRPLAPTRVAPRCPPSSAPGSAPAGF, 50, driver, 4.3, % positive cancer, 5.37, % positive normal, SEQ ID NO: 6272); AKR1C3, Del, A_16, KKKKKETQAKWPCASNCSKRWWSWSRASMRRESKRTSRFLTLN, 43, high expression, 5.44, % positive cancer, 6.61, % positive normal, SEQ ID NO: 6273); AKR1C3, Ins, A_16, KKKKKKKPRPSGLVLPTVARGGGPGQELQ, 29, high expression, 4.58, % positive cancer, 3.31, % positive normal, SEQ ID NO: 6274); CDC7, Del, A_7, KNMSRILNYQVLKKISRSFMKLYHSLVICLRLRTKLEKALLALFIWPQHSYE, 52, 16.62, % positive cancer, 9.09, % positive normal, SEQ ID NO: 6275); RECQL, Del, A_7, KIQVTSGSLQIRFSNLRIQGLRKEKLMMH, 29, 4.01, % positive cancer, 2.48, % positive normal, SEQ ID NO: 6276); ANKRD49, Del, A_7, KRWKKIQANCFFGLLKKIGLLQCGGYCLKRLLM, 33, 7.45, % positive cancer, 3.72, % positive normal, SEQ ID NO: 6277); GAP43, Del, A_7, KMMRTKRLNKMASNQKIKLIRPRPKFRLASVDT, 33, 8.31, % positive cancer, 5.37, % positive normal, SEQ ID NO: 6278); TM9SF2, Ins, A_7, KKKHVTELSTSLDC, 14, 4.01, % positive cancer, 5.37, % positive normal, SEQ ID NO: 6279); ANKRD49, Ins, A_7, KKDGKRSKQIASLGC, 15, 6.02, % positive cancer, 4.55, % positive normal, SEQ ID NO: 6280); DGKZ, Del, C_7, PPSGDLGGTRCPPKVKS, 17, 1.72, % positive cancer, 2.07, % positive normal, SEQ ID NO: 6281); EVL, Del, C_7, PHPHPHCQPEEHRVPAMTRAPCPAWLPPWLGPS, 33, 3.72, % positive cancer, 2.48, % positive normal, SEQ ID NO: 6282); GNA15, Del, C_7, PLPPPPARRGCTPHLQNRARSCKMGEGTSLLIPLLPPTTPVWS, 43, 5.73, % positive cancer, 4.96, % positive normal, SEQ ID NO: 6283); HDAC6, Del, C_7, PPTTPASPRSEVLRRVPYATPALV, 24, 2.58, % positive cancer, 2.07, % positive normal, SEQ ID NO: 6284); HSF4, Del, C_7, PLKVWSLQGPWMCWAPAIKGENGP, 24, 6.59, % positive cancer, 4.55, % positive normal, SEQ ID NO: 6285); INPPL1, Del, C_7, PHPRTASITLPTTSLKGSPTNCCPRSHPRLPGPLSHRPPRTKWPSQCLLHSSGATGPPVWERGAHQM RSLGAHCPLQTFHPHPCQTRPSSCPPAWILCQGRWSGAAVGVRPVAHHLTRPIQDLHCPQAPHLPA LSWGR, 138, 11.46, % positive cancer, 9.09, % positive normal, SEQ ID NO: 6286); PGF, Del, C_7, PPSNWPCLLEMAHQRWKWCPSSKCGDAATAGHWRSWWTSCRSTRMRWSTCSTHPASPCCAVVA AAETKTCTVCRWRRPMSPCSS, 84, 8.88, % positive cancer, 5.37, % positive normal, SEQ ID NO: 6287); SAMD10, Del, C_7, PPAPSAATGPGSRSRPARSRPSRAPRGRPSPRPQPGGPAVPQFTPPPEGAPAGAPPLPPRGRRNTARV RPLPPHS, 75, 6.88, % positive cancer, 6.2, % positive normal, SEQ ID NO: 6288); SORCS3, Del, C_7, PHPSARHLPPPQAGPGRPSPGRGSCSCRHGSWPAPRSLGERRAVLGARQPRLRGRRHRFLSSRGRR, 66, 4.58, % positive cancer, 4.96, % positive normal, SEQ ID NO: 6289); TEAD2, Del, C_7, PHLLTSQGTSLPKPCHPLPCPHLPHHPQPGRLGPWALPGCSWWSSQPLWNRPMQLILTRGTCLYTS ASTAPALAPPPSRVWTSGRSMTSSQRKRVAFGSCTIAGPLTPSSWSSSGRTSTGARVARR, 126, 5.44, % positive cancer, 7.02, % positive normal, SEQ ID NO: 6290); CHST2, Ins, C_7, PPPAAPGRACSRAARPALAVAPAPGPSLARVPARNEGVPQEGAGAVRGLRAAAGAHHAQPPGLQV AQGAAAAVRPRRAAGRRGGGCWGRLGAPGPPPGGAAPRTHPPGPPYSVPPSCRRRGGPRRCSRRR RRRRSRRQWHSGPRGWRRQEAVGVRVHHVALGLIVLR, 167, 9.17, % positive cancer, 6.2, % positive normal, SEQ ID NO: 6291); EVL, Ins, C_7, PPTPTPTASRRSTGCQP, 17, 9.17, % positive cancer, 4.55, % positive normal, SEQ ID NO: 6292); MIER2, Ins, C_7, PPAAATSRAQCACAGGGRGRGGPRGGRGARGLGAPPPPPAGPGLGPAPAALPPAPCPRGPPTSRSA LPGRAVGTPGGTRTPARPERTRRGPSAGPDQPYL, 100, 7.74, % positive cancer, 3.72, % positive normal, SEQ ID NO: 6293); PGF, Ins, C_7, PPPAIGLVCWKWLIRGGSGALPASVGTQLLPGTGEAGGRLVGVPG, 45, 6.3, % positive cancer, 6.61, % positive normal, SEQ ID NO: 6294); SAMD10, Ins, C_7, PPPPPARPPARGPGPAPRAAARAGLPEAGRLRARSPAALRCLSLPRRPREPPREPPRSRRAAAGIPRE FAPSPPTV, 76, 6.88, % positive cancer, 4.96, % positive normal, SEQ ID NO: 6295); AXIN, Del, G_7, LGGVATGTPVPPPEPTRSPRTPPCLP, 26, 7.16, % positive cancer, 3.31, % positive normal, SEQ ID NO: 6296); SETD8, Del, G_7, GGGGGLRNLPRPTPTSLQESHALASPF, 27, 3.72, % positive cancer, 2.89, % positive normal, SEQ ID NO: 6297); STOML1, Del, G_7, GARGPGPMRPRAGPPTSATASSASWGSCCCCSPSPFPAGSP, 41, 12.89, % positive cancer, 3.31, % positive normal, SEQ ID NO: 6298); SETD8, Ins, G_7, GGAGGGCGTFPDPPPRVSRNPMPWLPLSKVRAPAGLPLQGRRIHMSKLFGN, 51, 5.16, % positive cancer, 2.07, % positive normal, SEQ ID NO: 6299); TSPANS, Del, U_7, FFLCSWELFSSWSSLLGFWHLFSKTGSKTSCISL, 34, 6.02, % positive cancer, 4.96, % positive normal, SEQ ID NO: 6300); ABCE1, Ins, U_7, FFGWLRSNRKLEIQRCLTCF, 20, 5.73, % positive cancer, 5.79, % positive normal, SEQ ID NO: 6301); SLC17A5, Ins, U_7, FFWFLRSLCITCESECCISGYGRFKYNFSR, 30, 2.87, % positive cancer, 1.65, % positive normal, SEQ ID NO: 6302); TSPANS, Ins, U_7, FFFCVPGNYFLPGAHCWGSGICFQRLDQRPAVFLYKQQHQSVQR, 44, 4.3, % positive cancer, 6.61, % positive normal, SEQ ID NO: 6303); HELLS, Del, A_8, KKIKRRMRKKAPLLISV, 17, 7.45, % positive cancer, 4.13, % positive normal, SEQ ID NO: 6304); LIG3, Del, A_8, KKLRTSQSWKAGKSWKIMRRNR, 22, 1.43, % positive cancer, 1.24, % positive normal, SEQ ID NO: 6305); MSH3, Del, A_8, KKGEHFHWNRGSAACHR, 17, 4.87, % positive cancer, 4.13, % positive normal, SEQ ID NO: 6306); MSH3, In, A_8, DKKKRGTFSLESWECSLSQVRLCLIVSKTLLPVRS, 35, 14.04, % positive cancer, 5.79, % positive normal, SEQ ID NO: 6307); NUCB2, Ins, A_8, KKIRTRNSSIRAWWKIEV, 18, 1.72, % positive cancer, 2.89, % positive normal, SEQ ID NO: 6308); GPT, Del, C_8, PPHPPPPPTPRGRRRGRRCWRSWRQRRSSRSRCSTSPRASAATRCRAPCTPSLACSCPPAPCSALRN, 67, 10.32, % positive cancer, 7.02, % positive normal, SEQ ID NO: 6309); LOC106558478, Del, C_8, PPAPPPRCPPGRVRAELPAAGSAHPRGGPGRGVSSEATGAQVPALRAPVRIAGRAGR, 57, 4.87, % positive cancer, 6.2, % positive normal, SEQ ID NO: 6310); SIX5, Del, C_8, PPHPNTGPPNPDSPEPPPPPHPPPLLPPGPSIPAPLLPCRTPTPRGCSWGLRQGARRMRGWRPSRRF, 67, 11.75, % positive cancer, 5.37, % positive normal, SEQ ID NO: 6311); SLC25A29, Del, C_8, PPLSGPARPRAPSHAWAPRALPCRSASCCGRPPPGTGRAPHAAWVWQACLWDTHLTRSRCGCRCR ARRSLSTGGPCTASSPSSSRRACWACTRAWARRSWGSRSSTRWCSACRATPCGRWAATRR, 125, 6.59, % positive cancer, 7.44, % positive normal, SEQ ID NO: 6312); VASP, Del, C_8, PPHPLVCPSQGALRQGTEQGEAHPLRPLSPQHKAPVVGEQGPPVLQQPLPEPNSGKSASKRRPQGGP QSLKQRALEARAGG, 81, 10.6, % positive cancer, 6.2, % positive normal, SEQ ID NO: 6313); VASP, Ins, C_8, PPPTPWCVPLRGLCGRARSRGRPTPCAPSPHSTRHQWWGNRGPRSCSSHCRSQTQESQQARGGLRG APSP, 70, 17.19, % positive cancer, 7.85, % positive normal, SEQ ID NO: 6314); BAX, Del, G_8, RMGERHLSCPWSRCPRMHPPRS, 22, 11.17, % positive cancer, 5.37, % positive normal, SEQ ID NO: 6315); CLDN6, Del, G_8, GGAGAQVIIWPDTQHLPHMPPLRVPPSTPLRIMS, 34, 3.44, % positive cancer, 4.96, % positive normal, SEQ ID NO: 6316); DOC2B, Del, G_8, GGPPPTPRRKTRTPRVKIPTPPKIVLPWARWISACSTTRKTTPSTVPSARPRA, 53, 4.01, % positive cancer, 6.2, % positive normal, SEQ ID NO: 6317); PLEKHM2, Del, G_8, GGSSAVAVGGPTPRIGPTPSRSSSPTGRAWS, 31, 13.18, % positive cancer, 5.37, % positive normal, SEQ ID NO: 6318); CLDN6, Ins, G_8, GGGPELKSLYGPILSICRTCHLSVCPRVPH, 30, 6.02, % positive cancer, 5.37, % positive normal, SEQ ID NO: 6319); DOC2B, Ins, G_8, GGAPPQPPGGKPGPRGLKFRRLQKLYCPGHAGFQPALRPGKQRPPLYHQQGQGPEADGPQRASRPL CQAASAARSQ, 76, 5.16, % positive cancer, 3.31, % positive normal, SEQ ID NO: 6320); PLEKHM2, Ins, G_8, GGGAVRWLSEGQHHGSAPRLPGHPRRPAVPGAERRERG, 38, 5.16, % positive cancer, 4.13, % positive normal, SEQ ID NO: 6321); C4H5orf22, Del, U_8, QSRIPSKKCSLRKSTKSYKSCTSLKSLVPT, 30, 2.15, % positive cancer, 3.68, % positive normal, SEQ ID NO: 6322); INHBB, Del, U_8, FFMPTSISTRAHIRRLCGFFSPLGAKTRRMASPPPGSACTSSSPTKATRTCLWCRPACGFT, 61, 10.03, % positive cancer, 5.79, % positive normal, SEQ ID NO: 6323); FGF14, Ins, U_8, FFFQSGLFGPQEAQVEISGSPAQGYSDQVILQARLLLANAPRWSSRWNQG, 50, 3.15, % positive cancer, 2.07, % positive normal, SEQ ID NO: 6324); INHBB, Ins, U_8, FFLCQRLSQRARTYGDCAAFFPPWEQKRGEWPRLLPGPPVLLRLQRRQPEPVCGAGQPVALPEAPA LRPGEGQPEEGAGQGILPGAGPGRPVECGGEEGGPQAQRLAHLPTHGGHPGLV, 119, 3.72, % positive cancer, 6.61, % positive normal, SEQ ID NO: 6325); CCDC40, Del, A_9, KKPAKSSRRKSSLWGHNTTFD, 21, 1.43, % positive cancer, 1.24, % positive normal, SEQ ID NO: 6326); CEP57, Del, A_9, KKCKSWKQNSGKNNRKGNVCKLRQPSCRLG, 30, 1.15, % positive cancer, 2.89, % positive normal, SEQ ID NO: 6327); GRB14, Del, A_9, KKNTEHRLTMDSALSLTKREGPET, 24, 4.87, % positive cancer, 3.72, % positive normal, SEQ ID NO: 6328); KCTD16, Del, A_9, KKKPLRKSSQLRRSWRNASRIS, 22, 2.58, % positive cancer, 2.89, % positive normal, SEQ ID NO: 6329); POLD3, Del, A_9, KKGGSEWSYLMMKQKKLKI, 19, 4.87, % positive cancer, 2.89, % positive normal, SEQ ID NO: 6330); RGS12, Del, A_9, KKNIRKLIWTKQRSFLSSFPKLRATEQTTSAGC, 33, 4.87, % positive cancer, 4.55, % positive normal, SEQ ID NO: 6331); TAF1B, Del, A_9, KKGLQVFSSTGLKETLQEPVSTDMASRGF, 29, 8.02, % positive cancer, 4.13, % positive normal, SEQ ID NO: 6332); DNAH7, Del, A_9, KKNNCQPVTAVPNWNLFSTVRQHL, 24, 5.16, % positive cancer, 2.89, % positive normal, SEQ ID NO: 6333); GLRA4, Del, A_9, KKPCGNSMWTKPSRLTPSPGLSSLSLSSSSTPSTGLSIKCYGQKIFTRHC, 50, 7.45, % positive cancer, 3.72, % positive normal, SEQ ID NO: 6334); LOC100682643, Del, A_9, KKLSPPVPPISLWSLSIMAPLSLPMFTLQRSTTSPLVK, 38, 6.59, % positive cancer, 4.13, % positive normal, SEQ ID NO: 6335); NDUFAF2, Del, A_9, KKEKILKLKVNIFMKKKNSLAKGARKNSWLNQFKLRSKGMPRLHTLERKNLPWLPPALVKSFNRD PGCHKAARTQIN, 77, 5.16, % positive cancer, 4.55, % positive normal, SEQ ID NO: 6336); OPHN1, Del, A9, KKNLRRMVRGFIPCWIGTYIYLPKRKNLSYKRQTSRWTRKGTISLSPLLIMFIKFRKFRSPRSSTLWS QCWPFFIASSFPTV, 82, 17.77, % positive cancer, 15.7, % positive normal, SEQ ID NO: 6337); RAP2C, Del, A_9, KKRGGFAQSGPPPPTGKPPALSPLASSGAGAAVRPRSRPLAMALA, 45, 3.72, % positive cancer, 5.37, % positive normal, SEQ ID NO: 6338); ARV1, Ins, A_9, KKKTQLHFAAESIIIIQLWKALAHSSCHLGA, 31, 9.46, % positive cancer, 4.55, % positive normal, SEQ ID NO: 6339); CCDC40, Ins, A_9, KKNQRRAPAGSLPSGGTTPPSTEPGEQQYLLRL, 33, 3.72, % positive cancer, 2.89, % positive normal, SEQ ID NO: 6340); CEP57, Ins, A_9, KKNARVGSKTPGRTTGKETYAS, 22, 1.72, % positive cancer, 2.07, % positive normal, SEQ ID NO: 6341); ABCA9, Ins, A_9, KKKSSLTATDFRPLPFCLLVWPGTGGYSTLLLDPPSNANNGLCF, 44, 1.43, % positive cancer, 1.24, % positive normal, SEQ ID NO: 6342); CRYBG3, Ins, A_9, KKNPRAWWRGCWRNGERSP, 19, 4.58, % positive cancer, 2.48, % positive normal, SEQ ID NO: 6343); PAPOLG, Ins, A_9, KKKTASSLPSSRDSSKEEKAKSF, 23, 2.58, % positive cancer, 1.24, % positive normal, SEQ ID NO: 6344); RAP2C, Ins, A_9, KKKEVASPRVGRRRPPESPRPFPLSRAAALGRR, 33, 4.3, % positive cancer, 4.96, % positive normal, SEQ ID NO: 6345); CASKIN1, Del, C_9, PPPRRPRRRAPAASWTTSAACSTTWPTSWTPCWS, 34, 2.58, % positive cancer, 2.89, % positive normal, SEQ ID NO: 6346); PDZD4, Del, C_9, PPPSAMSIMTRRSSWRAARRRQTVWTSWSMRRWSCTRPATGTS, 43, 4.3, % positive cancer, 2.48, % positive normal, SEQ ID NO: 6347); SMG5, Del, C_9, PPQGRAASPRRRSSIPSGFTGLWWRLCIDSTSSFATKLLIKKCLNQRTLA, 50, 3.15, % positive cancer, 1.24, % positive normal, SEQ ID NO: 6348); ADAMTS17, Del, C_9, PPLGQEALTAWEPVWSMLSARTCPALRACPASETSSARHMTA, 42, 2.58, % positive cancer, 2.89, % positive normal, SEQ ID NO: 6349); B3GAT3, Del, C_9, PPQPAPGESCTLLMMTTPTAGNSLRRCAGPVVSQCGPWGWWAACDLRALRYRMAGLWASTRHG SPTGPSQWIWQDLLLPCPCCWLSPMPNLMLLLPGATWRAVS, 104, 10.6, % positive cancer, 5.79, % positive normal, SEQ ID NO: 6350); C7H1orf105, Del, C_9, PPRGESAFPRPTELRGSLLPPAGDPRNSRLLSRGRAARSLGKSKRRNERLDYLGSERRGTQAGLALN WGLATHPCIRWTGIRRAPASTVLGGLLTPQFLFRNLARFLGLARPAS, 114, 10.6, % positive cancer, 8.68, % positive normal, SEQ ID NO: 6351); CAMSAP3, Del, C_9, PPPAPLRCWPFWRGGAPCPRCPSALCRRRT, 30, 3.44, % positive cancer, 1.24, % positive normal, SEQ ID NO: 6352); CHST2, Del, C_9, PPRRRRPAHTPAWTPVLRTALLPPSGRPPPLQPPPPPPPQPPAMALGAPGLAETRGSWCTCSPRGAR AHRSSVSSSTRTPRCSSSTSRCGTCGKNCTRGTRFPCKGPRGTC, 111, 20.63, % positive cancer, 10.74, % positive normal, SEQ ID NO: 6353); COLCA2, Del, C_9, PPAARPGLASRPRTCRGQSLRRSLAWPGGEGRDSGLPRAVGQVTCYGSEKARCAGRGLSQAIAPLP RQQAGPRAEIPLI, 79, 11.75, % positive cancer, 7.44, % positive normal, SEQ ID NO: 6354); EPS8L2, Del, C_9, PPPRTRPPSPSSATAVTPQPARTGWARRCRSVSQASAVGSRRMRSPEPCWPRR, 53, 8.88, % positive cancer, 5.37, % positive normal, SEQ ID NO: 6355); FAM83H, Del, C_9, PPQRGRARHLTLSPRGAPPLPTRSGKGAPPRPSRSAGPARCPPCPSAGPAQCPPCPSAGPASPLPSPRS PPRPAPRRSRRAAPWRCCARAPCASASC, 97, 12.03, % positive cancer, 8.26, % positive normal, SEQ ID NO: 6356); FRS3, Del, C_9, PPTTTTTTRALLSARPSPSAPMRTSVRGCGQGRAGD, 36, 4.87, % positive cancer, 6.2, % positive normal, SEQ ID NO: 6357); LOC485980, Del, C_9, PPPLGGFGLLATGCPPGVSHGALKIWPGDAARRRPCRPGEACWARVALTVRARQSGAGCGLKTTA SSYKFGFVSAKVPEPRCCRDGSCGQ, 90, 6.3, % positive cancer, 6.2, % positive normal, SEQ ID NO: 6358); MADD, Del, C_9, PPPQCLPALASRTWTGVRQKLERGLKRCCGPTA, 33, 2.01, % positive cancer, 4.13, % positive normal, SEQ ID NO: 6359); MED25, Del, C_9, PPLLDPSFGLRTLGPTPNCGVSSSTHLRLRLGCPHPKLPSTTSSHQGLQHCCHHTRAWGNPSWGPPS CTHHLPSPGPHSFPRELRCQVRCC, 91, 13.47, % positive cancer, 8.68, % positive normal, SEQ ID NO: 6360); PEAK1, Del, C_9, PPRSSSRAASCTGARGRGPGRPARRGPRTPRRPCSPTPAAGPRTPGPSAGYRSRASSAAGRQRRRRP GTRAGSRAGCWAWTARSSTCCRRPPCSATTGSPRPRAGPGTGRPWSSRTGATLPTASPARTSRPEPS PPGPRAGAAPPHPGRRGRLPATLSRSSSPASAAPAAGSHGTGGPRPWSCSRSGRQARDPSRAVSAAL P, 202, 6.88, % positive cancer, 4.13, % positive normal, SEQ ID NO: 6361); PRAM1, Del, C_9, PPQPSPHCPLAPGMSRASEEPQPQPQL, 27, 3.72, % positive cancer, 2.07, % positive normal, SEQ ID NO: 6362); PRR12, Del, C_9, PPLSRGSLVPAAMAHPSPRCWRRSHHPRRPLPRRHSRYHRPRHPLRQPCPRRLRWWPRSPAHRQPH RPCPRLRPHRHPRRLPPPPPPPSCPRSPQPRPPRSRSRPTPGPCTWPRSRKRRRCAGRPTRRLARAAG RASSGSETSSSSAPKTSLRSSWHCRRGGSPHPSGECRKPCCRNSLQRSRTGSGSFVLPVTIWGTLGTR KTGISAFM, 210, 7.45, % positive cancer, 7.44, % positive normal, SEQ ID NO: 6363); PRRC2A, Del, C_9, PPHLAERAALRPAGALAQAAVVVGSLQRNQGPLPAGLGL, 39, 6.88, % positive cancer, 5.79, % positive normal, SEQ ID NO: 6364); RHOBTB2, Del, C_9, PPPSLWCPIPRPAARSAPPTSWRTRCVRMSYWCCRSGCASLPTRSTSPPRPPSSTTCSSWT, 61, 1.43, % positive cancer, 2.48, % positive normal, SEQ ID NO: 6365); RIPK2, Del, C_9, PPVTQTSPSPPPPPRPGPAVTSRGQLGEGGARGGRGAASTRRRRRGAGGPGKRSPLALRDSGGGWR APRGHGLPGHLVPRRGKGPAAPAPAPAPAPAPAPAAIGLPLPLRLPFSPGSRSAPAPALAPAPSPAPA PPPQLPSSSSSRSHPRLPLLSPAPAPAPGPSSRCSLRLPLPSPAPAPAPAPGSRPQLPLQAPAPAPGPGPP PSAEPPSLSGNLRRGGHTRAAAPRP, 230, 18.91, % positive cancer, 13.22, % positive normal, SEQ ID NO: 6366); SAMD10, Del, C_9, PPHPGGPPAPSAATGPGSRSRPARSRPSRAPRGRPSPRPQPGGPAVPQFTPPPEGAPAGAPPLPPRGRR NTARVRPLPPHS, 81, 1.43, % positive cancer, 1.24, % positive normal, SEQ ID NO: 6367); TMEM253, Del, C_9, PPLFCPPLAIPPTASPGKSSPWVL, 24, 2.58, % positive cancer, 1.65, % positive normal, SEQ ID NO: 6368); TRIOBP, Del, C_9, PPLPCSQPPLLHSGRSIGLC, 20, 1.72, % positive cancer, 2.48, % positive normal, SEQ ID NO: 6369); ZCCHC24, Del, C_9, PPPTTCATCASTKDTTSRTVPRHAPKARV, 29, 1.15, % positive cancer, 2.07, % positive normal, SEQ ID NO: 6370); ZNF219, Del, C_9, PPRSRRPLPRGARHSRQGPSPPRSRPPGQRAPRTPGLPRARRRPGPVGSPPVPGGPCATGEAVRPNP WTCPCGRGRGARPGRGAPSTGASSAPSPPEPRSSWPCTCKCTTAAGLGAAGRPRRMRPRPTPPHSP QRPPPVLRRRGRRAPGCRGPRSG, 156, 12.03, % positive cancer, 7.85, % positive normal, SEQ ID NO: 6371); ZNF628, Del, C_9, PPNLAKPLPQWASPQLPPPRWYKWYPLALHLEV, 33, 0.86, % positive cancer, 2.07, % positive normal, SEQ ID NO: 6372); ZNF865, Del, C_9, PPHRPSLMLPSPPRNGASSTSLDTSTFLGI, 30, 2.87, % positive cancer, 2.07, % positive normal, SEQ ID NO: 6373); CASKIN1, Ins, C_9, PPPLVGRGEEHRQHPGRHRQHVRRPGRPAGRHAGV, 35, 1.43, % positive cancer, 1.65, % positive normal, SEQ ID NO: 6374); SMG5, Ins, C_9, PPPRGEQRARGEGPPYQAALPGCGGGCASTRPHPLQQNCLSRSV, 44, 3.44, % positive cancer, 4.96, % positive normal, SEQ ID NO: 6375); C7H1orf105, Ins, C_9, PPPAARAPFPGRRNCGGRCCPLPATRGTPGSSPEDAPLGA, 40, 2.29, % positive cancer, 3.31, % positive normal, SEQ ID NO: 6376); CAMSAP3, Ins, C_9, PPPQPLCAAGPSGEEGHRARAARAPCAGGGPEAPAYPHDHPAAAGED, 47, 2.01, % positive cancer, 4.13, % positive normal, SEQ ID NO: 6377); COLCA2, Ins, C_9, PPPPPVRGWLPDQGHAEVRASAGASLGLVAKVGTRGFHARWAKSPATDQRRPGAPAAASLRPSHL SPASRLGPGPRFP, 78, 7.16, % positive cancer, 6.2, % positive normal, SEQ ID NO: 6378); GNA15, Ins, C_9, PPPRPPAGAARPTCRTGQEAAKWGKGLRF, 29, 1.72, % positive cancer, 1.24, % positive normal, SEQ ID NO: 6379); GPT, Ins, C_9, PPPPPVAAGEAGGAGGAGGKGEAHGAGVQRVPGHPLQPGAGRHVLLPSRAAAPPRRAARSGIEPG PRHVLLHAPPGGDRHLRGARQRLRAEGRHLPLPDDHSAPHGEAAASAGEAEPVPRQVHPRVLL, 128, 11.17, % positive cancer, 10.33, % positive normal, SEQ ID NO: 6380); MADD, Ins, C_9, PPPHSASQHWQVERGQASDRNWRGGSKDAAAQQLETGKRLRRRVRLSSELSHLHRLQHQHRGLR GHHVFCQQPVSKPQYKLQSFKPHTADQRCPREDDPLPQSERKQEGLSGPEVICY, 118, 1.15, positive cancer, 1.24, % positive normal, SEQ ID NO: 6381); MED25, Ins, C_9, PPPSWTHPSASEPWGQPPTAESPPQPTSASDWGAPTPSFPPPPPATRGSSTVATTPGPGATPAGAPPP APTTCPVLAHTASPESSAARSDAAERGSPGPGSPAGPAAQRHGGRHPHGSHL, 120, 14.61, % positive cancer, 14.46, % positive normal, SEQ ID NO: 6382); PEAK1, Ins, C_9, PPPAAAAGQPPAPAPGAAGRAGQPDEGRGRRGGPAPRRRPPGPGRQAQALDIVQELLPPPEGRGGG GQGQGPGAGPAAGPGRHGHPHAAAAPRAAPPLVRRGPGRVPGQAGPGLHVQVRPCPRPARRAPA GPSRAPRAREPGPLLPTPAAEAGSQPLCPEAAVRPQPHPRRARTAQAGRGPGAAAAVAGKPGTPQG Q, 197, 10.6, % positive cancer, 10.33, % positive normal, SEQ ID NO: 6383); PRAM1, Ins, C_9, PPPSQAPTAPWPQGCPELPKSRSRSHSSEEDPFFCWDPPPSPTDSRHPAGPGRGL, 55, 2.29, % positive cancer, 2.48, % positive normal, SEQ ID NO: 6384); RHOBTB2, Ins, C_9, PPPHHCGARSPVQQRGVPRPPPGGPAVCGCHTGAAGAGAHLCPQDLPLHLVLQVLRPVPHGPK, 63, 5.73, % positive cancer, 5.37, % positive normal, SEQ ID NO: 6385); SAMD10, Ins, C_9, PPPTRGAPPPPARPPARGPGPAPRAAARAGLPEAGRLRARSPAALRCLSLPRRPREPPREPPRSRRAA AGIPREFAPSPPTV, 82, 3.44, % positive cancer, 1.65, % positive normal, SEQ ID NO: 6386); SORCS3, Ins, C_9, PPPRRLARGAPRPDGAPAPVDMDPGRRRDHLASDGRSWAPGSPGFAAAGTASSPPAGGGKPVAGG AGDRARRLRAGAPARARAAAPAGRRRRRRRRRRRRRQRWKEAGRSRGAVARRRAVGTRHPGSG QGWRREEESPGAAPQRPGTRGRPGPRPSRWCPRKPRSGQGSPGGGEGAAGRGGGGRRAPAAQHL VRADRGLGPQSGHGALVGTQQQRYTYPDEAV, 223, 7.45, % positive cancer, 7.02, % positive normal, SEQ ID NO: 6387); TMEM253, Ins, C_9, PPPSSVRLSLSHPPQARGNRVPGCFSLPPSIRFLAPLAKTTSPRMPLAAPGPAGK, 55, 2.87, % positive cancer, 2.07, % positive normal, SEQ ID NO: 6388); ZCCHC24, Ins, C_9, PPPQLPVPPVLQQRTLHQGLSPGTPQRRGSDPVPGQKALFRRVQVSQVQEKMDERELLGQHGAGV H, 66, 3.15, % positive cancer, 2.89, % positive normal, SEQ ID NO: 6389); ZNF219, Ins, C_9, PPPGAAAPFPAGLGTAVRGQARPAAGRLGRGRRAPPASPERGGARVPSEARQSREDPAQRARR, 63, 9.17, % positive cancer, 9.92, % positive normal, SEQ ID NO: 6390); ZNF628, Ins, C_9, PPPTWPSLCPSGHLHSSHLPDGTSGTHWRCTWRYDPTGPTLHPDCPDSARSPAGAYIL, 58, 2.01, positive cancer, 2.48, % positive normal, SEQ ID NO: 6391); MAPK11, Del, G_9, GGPGHPQTCPTATSHQARPTTHGCARRWR, 29, 5.73, % positive cancer, 0.83, % positive normal, SEQ ID NO: 6392); ABCA13, Del, G_9, GGHLGMTWSILRRTAVGTASCGPPGAPVPPAAGPSGAEGGPWASVAPPQGPPWPRPRPSW, 60, 8.31, % positive cancer, 7.85, % positive normal, SEQ ID NO: 6393); ADAMTS3, Del, G_9, GGKAGNTGPGTGGSSGNSWATGAGFSPSWKGPNPSWTTRTGPGSGRTTAGPATGCPAPTSGKRAL E, 66, 10.03, % positive cancer, 8.26, % positive normal, SEQ ID NO: 6394); ADAMTSL3, Del, G_9, GGRGLRRGPHGPGRRVLPEGRRGAPGPLARPRPPLPSAPAGGGGRGGVGNPGSGHHAWRPAQGEE WLPGSGRRSVPAASRRRRGKKRSERPGPGPSRATLAPRRPRALGSGALGGGQPGARSVARSGRWP VGRERGSRGRGRAGGASAGRRAAGGARGPGPGPGTGRLPSAPSRRRRWREATDPPPDTGSAKAPA RRSERRAQERFPRRLDPMASWKSSWWLLMWMVFMHSALLQAD, 237, 7.74, % positive cancer, 9.92, % positive normal, SEQ ID NO: 6395); CCDC85C, Del, G_9, GGGRPPEPSPLGHGPGPHRARTPSGARSRSPSAGSGGGRAWSRGHGRGQRAALPQGRATPESRSAR RWRGAGAGAARAAAAARAPAAPSTARPA, 94, 11.75, % positive cancer, 7.02, % positive normal, SEQ ID NO: 6396); FGF14, Del, G_9, GGGAAPARPGGFATAPWGIFFPKWAFWPSRSAGRDLRIPSSRV, 43, 9.46, % positive cancer, 7.02, % positive normal, SEQ ID NO: 6397); LOC106557553, Del, G_9, GGEVKGPQESKEDRHRLPTYSLPGPARPQGRPLPPAEWNPSTPGASPVTTVRIRVEMTAGVLTCFV, 66, 10.6, % positive cancer, 5.79, % positive normal, SEQ ID NO: 6398); LOC106559695, Del, G_9, GGGVCGGADFALCGRAGRTGRPGPVPSALSPVLREARAPGGRARAAQDIS, 50, 1.15, % positive cancer, 4.13, % positive normal, SEQ ID NO: 6399); LOC477678, Del, G_9, GGDSGVSLVLRRKRGPQISPASPWLFSFHQKISS, 34, 6.59, % positive cancer, 4.96, % positive normal, SEQ ID NO: 6400); NCKAP5L, Del, G_9, GGRAPRAGGTTGAPPQPSSYSKC, 23, 6.02, % positive cancer, 2.07, % positive normal, SEQ ID NO: 6401); PRSS16, Del, G_9, GGVFARAVSRSRSAGRRRPQPSQRWNDGCTRAGRLARRCGRSWAPAHLWTEPRTRRSCWGRCRP WWGAPCSTTRRPGCR, 79, 12.89, % positive cancer, 6.61, % positive normal, SEQ ID NO: 6402); RAMP1, Del, G_9, GGMWGGSSRRRRSAQEDQEQQPPGRRSRCWCSCTTSSWPRPARTPTTALSSRTCASPASR, 60, 9.17, % positive cancer, 7.44, % positive normal, SEQ ID NO: 6403); SALL3, Del, G_9, GGCWGPRRPPGAPARGRPGPAGRRGRVGARERRARAGEAGAGAALFGAGPRGRCPPSGLPLSRAG GRRSAVGGRPAFLVAAWGPGARRGGP, 91, 16.62, % positive cancer, 8.68, % positive normal, SEQ ID NO: 6404); SALL4, Del, G_9, GGGQTTLPRGLVASKGVGPPSRGQRP, 26, 5.16, % positive cancer, 2.07, % positive normal, SEQ ID NO: 6405); TMEM55B, Del, G_9, GGPAAGAPQPPFPVQRSPRSPRDTRPCSRARTRPPTRP, 38, 4.87, % positive cancer, 5.79, % positive normal, SEQ ID NO: 6406); ULK3, Del, G_9, GGVGGHRLCPSPRTCRPRRTPARS, 24, 2.58, % positive cancer, 2.89, % positive normal, SEQ ID NO: 6407); MAPK11, Ins, G_9, GGGRATHRRAPRPRPTKLGLRHTAAPEGGGEETVAPLPVADPRTENLPRAAPAQASEA, 58, 6.59, % positive cancer, 3.31, % positive normal, SEQ ID NO: 6408); ADAMTS3, Ins, G_9, GGGRLGTLDPELGEVPVTAGQRGLALAPPGRGPTLPGQQGLARGAAGPPPALQPGALPLPVENGP WSEVGHLFRNLWGGHGGEAGPLQSGRPLRRGKARSRQTLSAARLQ, 110, 12.89, % positive cancer, 8.68, % positive normal, SEQ ID NO: 6409); ADAMTSL3, Ins, G_9, GGGADSAEVRTGRGAGCCRRGGGGRPGRWRGRGRLSPARLPAAAAVAASAIPALGTTPGGRPRA RSGFQEAGGGAFQPRLAVAEAKSAPSGPGPAPPALPSRPGAPGPWGAGPSGAGSQERGASQGPAAG PWGASAGAGAGDAREEPARGGGRPAGRAVPVPGPAPAASLRPRVAAADGGKPPTRPRTPAPPRRP RGEARGELKRDSPGDWTPWLPGRAPGGC, 223, 12.61, % positive cancer, 10.74, % positive normal, SEQ ID NO: 6410); CCDC85C, Ins, G_9, GGGAGPPSPPPWGTGRVHTVLGHPPARGAAAPPQAPAGDARGVGGTEGDKEPPCLKGGRPPSPGA RGAGGGRGRGRRGRRRRRGLPQLHRQPGQPERAPGRRRGGRGGPRRGRRQQHVERGQRRQPGPP PPRPAPAAAPRAAQGPRRQGGRHAQVPGRPVGATAPPEHPQRPARSLVHLHQAAGDQGEAAGG, 192, 12.61, % positive cancer, 9.09, % positive normal, SEQ ID NO: 6411); FGF14, Ins, G_9, GGGEQPQQDPGVLQRHPGGFFFQSGLFGPQEAQVEISGSPAQGYSDQVILQARLLLANAPRWSSRW NQG, 69, 3.15, % positive cancer, 2.89, % positive normal, SEQ ID NO: 6412); LOC106557553, Ins, G_9, GGGRSKVRKRAKKTGTGCQPTASRVPPGLRVGPCLLPSGIHPPRGPLLSRLCESVWR, 57, 7.45, % positive cancer, 5.37, % positive normal, SEQ ID NO: 6413); LOC106559695, Ins, G_9, GGGVFAEGPISPCVAALGGQEGRDLFQAL, 29, 2.87, % positive cancer, 1.65, % positive normal, SEQ ID NO: 6414); NCKAP5L, Ins, G_9, GGGGPRGQEGRLGLPLNPHPIQNV, 24, 4.3, % positive cancer, 2.48, % positive normal, SEQ ID NO: 6415); PRSS16, Ins, G_9, GGGSSPARCLAPAVPGGGVRSLRRGGTTAARGRGDSLGAAGGAGRLLTSGQSRGPGGAAGGAAG PGGGRRAVRRAGRGAAERARALRTPARGPEQPQPLRALPRAPAGSAGCHAQSRPEVFKLLPSRNSG TTEGHRFSPVGCG, 143, 15.19, % positive cancer, 11.57, % positive normal, SEQ ID NO: 6416); RAMP1, Ins, G_9, GGGCGEAAPDGGARLRRTRSNSPRDAGAGAGARAPPPRGHGLPGRRLRRSPPGPVPRPLQGEHGG HREDAVVRLGQDHRELRGAHGLHQARGGPAGLLLAERRRAQVLCRRAPALLQKLPRVRQGRA, 127, 12.61, % positive cancer, 9.5, % positive normal, SEQ ID NO: 6417); SALL3, Ins, G_9, GGGAGGRAAPRGLLRADDPGRRGDGDVSGLASAGRGPGRPAPGQLCSEPGPGAAALPPACPSPGP AVGGRRSAVARPSSWPPGDPARGGAVLERGGPGTLAAAPSPLGRVRTPRRRPAGVHPRRLGACPR RERGAGSGERGAGAGSGSRCRRSQGRAGAAGAPSSQRGSRPPAQNPLPNFCDGPFR, 186, 16.91, positive cancer, 9.5, % positive normal, SEQ ID NO: 6418); SALL4, Ins, G_9, GGGAKPHFPEGWWLPREWDPRAGVRDPEITAAGGEHRQGHHRPQRMSHLPPGLELPKLPQDALPH PHRGEAVPV, 74, 2.29, % positive cancer, 2.89, % positive normal, SEQ ID NO: 6419); TMEM55B, Ins, G9, GGGLQLVPPNPPFLCSVPPVPRGTPGRAPGRGPAPLLAPDQPGQRECPDDHLPCLPVSHQRGRQDAS ACSQMWRLQ, 76, 4.3, % positive cancer, 5.37, % positive normal, SEQ ID NO: 6420); ULK3, Ins, G_9, GGGSAGTGCARALGPAAPEGHPRGRSHKVRGQEEPEQGLGGEPPDGDRDPQGHSTPAHRAAPRLP VGQRPHLPHHGVLRRGRPVPLHPHPQASAREGGSRLHAAVG, 106, 2.29, % positive cancer, 2.89, % positive normal, SEQ ID NO: 6421); INTS2, Del, U_9, FFLSHLSYLRVQSIWRKLQMYFVFYKQSSLPCFL, 34, 3.44, % positive cancer, 5.79, % positive normal, SEQ ID NO: 6422); LOC100856768, Del, U_9, FFWKKTQFLIIYVPHRELST, 20, 2.87, % positive cancer, 2.07, % positive normal, SEQ ID NO: 6423); ABCC11, Del, U_9, FFRCLRHPSTCSRGWRTTAAPLCSPTFSPLFRA, 33, 3.72, % positive cancer, 2.07, % positive normal, SEQ ID NO: 6424); GABRR3, Del, U_9, FFISDITGKMRGSPFLAQQTKA, 22, 1.43, % positive cancer, 0.83, % positive normal, SEQ ID NO: 6425); SLITRK3, Del, U_9, FFLLSLLLQASLPMCCDGAGRSCPLEASGKKVWTLLASRCNATGFLRMVEVVEVEVEVGADQLFP PRRRLLRWATYMSTSPTPLPRCATTPSTSLVRRKRWLFPQSRRQGAQNEGVLGHNHQE, 123, 14.9, % positive cancer, 15.29, % positive normal, SEQ ID NO: 6426); TSPAN18, Del, U_9, FFFCSSSSSSWRSSRRPSWPSSSGKISLASPSPRSSPSTTRATMTQTSSPPPGTPS, 56, 8.31, % positive cancer, 7.44, % positive normal, SEQ ID NO: 6427); ZC3HAV1, Del, U_9, FFCLRYAKIIRERVEELFVTSIHSVKGSTSVSTLPEGIVGMPTASGPIT, 49, 12.32, % positive cancer, 7.85, % positive normal, SEQ ID NO: 6428); LRRIQ1, Del, A_10, KKIRHTDTQRDLQVSCGFLQS, 21, 3.44, % positive cancer, 2.89, % positive normal, SEQ ID NO: 6429); ENO4, Del, A_10, KKKDKSKEGMILSQRNLFCLQNLLNPYSVAAWP, 33, 6.02, % positive cancer, 2.89, % positive normal, SEQ ID NO: 6430); NME8, Del, A_10, KKKLSRQGTSWKLRTRECSQNNKQGSSIVAKRRSLTFKILSFL, 43, 4.87, % positive cancer, 5.79, % positive normal, SEQ ID NO: 6431); SREK1IP1, Del, A_10, KKKGKGLIHPVPLKRTLPSKRNKNTRRKKRRKKKRISQKEGNIIKRKKRREKRRSIHPLLILLRSPKS, 68, 12.32, % positive cancer, 7.44, % positive normal, SEQ ID NO: 6432); CEP290, Ins, A_10, KKKNSPNGSRKRKKSCNFRINHGGLEPN, 28, 9.46, % positive cancer, 4.55, % positive normal, SEQ ID NO: 6433); PDIA2, Del, C_10, PPPSPSGSICRLWPGEGRRGEPRARSGAPGPPRGSLASVGLSRASVIDPEGVASTLAPGPRGRVI, 65, 9.46, % positive cancer, 8.68, % positive normal, SEQ ID NO: 6434); BAIAP3, Del, C_10, PPPASSATRAAGAESSAGPRPPCSASTARRTTCLRCSWRCCTGR, 44, 6.3, % positive cancer, 3.72, % positive normal, SEQ ID NO: 6435); C5H11orf70, Del, C_10, PPPPRPRCVRSRPSLPSAEAPPRGSLSTARPPQTCGLLPSRSSEPGLHHTSTAS, 54, 12.32, % positive cancer, 6.61, % positive normal, SEQ ID NO: 6436); CBARP, Del, C_10, PPPTPSRPPRPTSPYSSPRGRPSPAAPWAPAPPCQVTPTAQPWALPTSRSAPWHPATAGKAPGPTQV RGAPWPWGRGPRAGPGRPAQAPGQDLSCSSSLACGATPAWTGRAPTSRSRNGSWTPASGHPAWIP EAPPSGTTSSGSGQPVRARSRRRATPPTWTSSNTSPAQATLWPSRPPTPFWPAPPAHPPLSAGIFQ, 198, 10.32, % positive cancer, 10.74, % positive normal, SEQ ID NO: 6437); DBX1, Del, C_10, PPHSPSLHASPLRRAPTPAGVGVGAPPRFPPPPPTRTRT, 39, 5.44, % positive cancer, 4.13, % positive normal, SEQ ID NO: 6438); DPH7, Del, C_10, PPPAALTGDFSPHPDRDPRSHRGPQLPHRPYSHRRPQPPPRPPLSTWPLHPAFAALSPLRLRLTPG, 66, 11.17, % positive cancer, 9.92, % positive normal, SEQ ID NO: 6439); EBF3, Del, C_10, PPPDPGPPDPLAARRTPRACSGTRTPPRRRPRAGSPALPCPALPCARQAADGAGRPPH, 58, 10.6, % positive cancer, 5.37, % positive normal, SEQ ID NO: 6440); HOXD8, Del, C_10, PPLLGILGLGRRLPPAAGLPVTGSPRSFTDTITYRDSRFLRPSKRPSWYNILTVNRPVVILARTQTT, 67, 5.73, % positive cancer, 7.44, % positive normal, SEQ ID NO: 6441); HSF4, Del, C_10, PPHCPWLWCRPSWKGRGTSAPRGPGMPNSLNQGVPGRYLTGGLWAWTGGHEAQRICCLPCCFGP PLKVWSLQGPWMCWAPAIKGENGP, 88, 5.73, % positive cancer, 4.55, % positive normal, SEQ ID NO: 6442); PIGM, Del, C_10, PPPSGVSGSGAAAGRAEQSRAEQSSGAPRPDAEVPPPPQEANPEGENGGVAPPPEAGRGVGGARAS GAGLPAGSGRGAGARGFGAAGLRGAGGGWAGP, 98, 12.61, % positive cancer, 9.5, % positive normal, SEQ ID NO: 6443); PXDNL, Del, C_10, PPPSARGRSRAAAAAAAAS, 19, 3.72, % positive cancer, 2.89, % positive normal, SEQ ID NO: 6444); PDIA2, Ins, C_10, PPPPPQAARSVGCGRERVGGESREPVPAPRVLRGGP, 36, 3.44, % positive cancer, 5.37, % positive normal, SEQ ID NO: 6445); BAIAP3, Ins, C_10, PPPPPPAQLEQLARRAQRARGHRALPPRRAEQPVCAAAGGAALGGEQPSPSDLHAGLRLPAGAAG GHAGTLGGGVLPAPRAGEEPG, 86, 5.44, % positive cancer, 5.37, % positive normal, SEQ ID NO: 6446); CBARP, Ins, C_10, PPPPRRRGPQGQPRHIPAPGEGPHRPLRGPQLRPAR, 36, 4.3, % positive cancer, 5.79, % positive normal, SEQ ID NO: 6447); DBX1, Ins, C_10, PPPTAPVSTLHPSAAHLHLRGWGWGPRPGSHPPPQPAPGPERRGPEGRGRGRGGRAPPQPPPPRAPR APRGAPAAPARPAAGPAAARLARALGQPRQTLGLLGLRGRGGGRGRGDHRVL, 119, 8.31, % positive cancer, 7.02, % positive normal, SEQ ID NO: 6448); DPH7, Ins, C_10, PPPPPLSPAISAPTRTATPALTADPSSHTDPTLTADLSPHPDPRFQPGLSTPPSPPCLRCVCV, 63, 6.3, positive cancer, 6.61, % positive normal, SEQ ID NO: 6449); EBF3, Ins, C_10, PPPRTPDPRTHSRRGGRRGLAAGHGHHRAAAPGPGALPCPALPCPARARPRTAPDARRTKCNYLSL LSGAQQRENQQRHPLQAPVAVQQRSQNGAGSLCTPHRFHDQTGHRVRGPGQEPGDVSRAADPRD NVQPVL, 136, 7.74, % positive cancer, 4.13, % positive normal, SEQ ID NO: 6450); HOXD8, Ins, C_10, PPPSSASSASAAASPLRRDCLSRGAREVLRIR, 32, 1.15, % positive cancer, 1.65, % positive normal, SEQ ID NO: 6451); HSF4, Ins, C_10, PPPTVRGCGAGHPGREGELQPRGAQECPTA, 30, 1.43, % positive cancer, 3.31, % positive normal, SEQ ID NO: 6452); PIGM, Ins, C_10, PPPPPASQARGPRREGRSRAEQSRAAGRRGQTRKSRPRRRKPIRRERTAAWLLPRKRGGAWAGRA RRGRGSLPEAAAARGPGASGLRGCGGRAGDGPGPEPAGAARRGVRRGLRGAGGPGAVRRLPGPG AARALHGRGLPGVHGRRALAVAGPLPVPARHLPLHAAAGLAADAQRAPVRAVREAAVHQLRPAG RLAALPPAAPARAGPARGLRLLRPVAAEPPPHGGVQPRQRGRAGGGAGAGGAVPAGARAAGVRR RGLRARRAPEAVPGDLHPAHRAAPAAGRAPARAPARARAPPRPCVRAAPGAVQPRGAAVRRGGR AHLLRPEPRLLPPLRLGVPGAHLPVPPDEAGHPAQLLALLLHAVPDGREPVEPCAGRRRLPAAAAA ALGRVPGLLQGPRLLLFPAHGDLRDFQQGLHLPVLPLVPLPAAPCDAAGEDALETRRRPPGAVVRR AGLVAGARLSPGVPGKEHLSVYLVGRFVLLPHQLLHPNSDHFPLQGGTPGRESQM, 508, 9.74, % positive cancer, 9.09, % positive normal, SEQ ID NO: 6453); PXDNL, Ins, C_10, PPPRVHEAGAGQPQPQPLLPSGGPQGQRAAGPHSHAHVVVSRAQQGGRGAVCPEPPLGRRHPLPRS QEDRGCPAAAHHLQPLAAQDPGGAGHEDAAGLPGLRPECERGDHQLFCYCSLQIWSHLDQSYSLP TERHFRGNPRRPSSPP, 147, 4.87, % positive cancer, 4.55, % positive normal, SEQ ID NO: 6454); SIX5, Ins, C_10, PPPPCCPPAPPSPPPSSPAGPRPRGAAPGVCGRGRGG, 37, 0.86, % positive cancer, 1.24, % positive normal, SEQ ID NO: 6455); SLC25A29, Ins, C_10, PPPPGPGGPHTRRGCGRRACGTPI, 24, 6.02, % positive cancer, 2.89, % positive normal, SEQ ID NO: 6456); AMER2, Del, G_10, GGGTAAAVGGRTRGPPGRRGPARAPWAAARWPSPTASSPCCARTGGRRAAGASRPTRARPAANK SEGSRGCSAACAGTGRTSAPRRRPRASARGPRAASSCPARSPPAWSASRRRRPRPRPRPRPRPPAPA RRSSPAGTRRRGQQAAEILSQTPRTRQAPAAPSMAPGPGSRAPLEGAPEWWPTKEEGRRWRARSPP PCIFRNKVLFLLKRPLQKERQGPGTRPRAGTRGSTAAPPSPRPRPRPRTSPRSRAASPRRPSPTATRA AGLPRPGPGPRRTARGPAPPGRAPAGTRPATPTPTPPPAPPETRRPARPA, 315, 14.04, % positive cancer, 9.09, % positive normal, SEQ ID NO: 6457); C29H8orf37, Del, G_10, GGGALGVASRCPWKPPGVAATRRGKTSRSKMAEDQQKHLRKKMILTVLLMKYLKNPTWTKNPL N, 64, 12.61, % positive cancer, 6.2, % positive normal, SEQ ID NO: 6458); CPM, Del, G_10, GGGIQGRSPRVPQPTPPGRPTPAA, 24, 2.87, % positive cancer, 3.72, % positive normal, SEQ ID NO: 6459); SLAIN2, Del, G_10, GGGSGRAPGGRTTTNFGKPPPCWRRARAACLTRWSRCGPTSWSGCRAGRRRRRAGCIHHQRKN, 63, 5.16, % positive cancer, 3.31, % positive normal, SEQ ID NO: 6460); SLC44A3, Del, G_10, GGGTRPPPGGRSGPGRPRGSDGGGSARRRCAPSRPCSPRGARVRASPRPAQRRMQRPGAPAPVSHR RGLLQREPPGQGSGDLSSIGSARIRPGCSCSSSSGLV, 103, 16.62, % positive cancer, 7.02, % positive normal, SEQ ID NO: 6461); ZNF282, Del, G_10, GGGVARAPKWPPPVPAGGQELGGHWLASWLRRGGGAAEGDCLLLRAVDGAVRPRRGGRRLENV LLLPGRPERGTGLPGCSLCQHGRSLSSWASRAWGWTAGAGAGPRLCPRRRSATRSRRCAGKWPRE CRPCMLKSGTWMPGDRCLFSSRPFRIGHLSSLTA, 162, 19.77, % positive cancer, 11.16, % positive normal, SEQ ID NO: 6462); AMER2, Ins, G_10, GGGGPRRQWAAAPGAPQAGGARRGLPGRQLGGQVPQLLLPAAQERAAGERQGRAGRRGQGRRQ TKARAQGAVQQHALAQEGQARQGGGQGRARGGPGRPHPARLAHRQPGVRPGGGARARARARAR ARPRQPAGAAQRGRAAAASRLRRYYRRPRGRGRPRPRPAWPRARAAGPP, 175, 6.59, % positive cancer, 9.92, % positive normal, SEQ ID NO: 6463); CPM, Ins, G_10, GGGASRAGAPGCPSPRHRDVPLPQPDPRRRRLPTPRPRLRLEKPHHNPPPPPPPPRLRLVSAQRWNA RLQLHLGAVL, 77, 4.58, % positive cancer, 2.07, % positive normal, SEQ ID NO: 6464); SLAIN2, Ins, G_10, GGGGRVGPPGDEQRPTSGSPLPAGGGRGRLA, 31, 4.01, % positive cancer, 2.07, % positive normal, SEQ ID NO: 6465); SLC44A3, Ins, G_10, GGGEPGLPRAGGRGRAGRGAATVAAPRGGAAPLPGPAPPAGPASAPVPARPSAGCSAPEPRPR, 63, 8.88, % positive cancer, 7.02, % positive normal, SEQ ID NO: 6466); ZNF282, Ins, G_10, GGGAWPGPRSGPRRSRRAGRSSEAIGWRAGCAGAGAPPKGTVCSYGL, 47, 5.44, % positive cancer, 6.2, % positive normal, SEQ ID NO: 6467); ASTE1, Del, A_11, KKKGKRDTVPAGQKTEWEPPQTLDAGMREAIGLGY, 35, 4.3, % positive cancer, 2.89, % positive normal, SEQ ID NO: 6468); EFHC2, Del, A_11, KKKYYPAKTSEGCASPSGYLCLMIFWDPYCPGLKTAKNK, 39, 12.89, % positive cancer, 7.02, % positive normal, SEQ ID NO: 6469); ASTE1, Ins, A_11, KKKKAKETRYQLVKKQSGNHLRH, 23, 9.74, % positive cancer, 4.55, % positive normal, SEQ ID NO: 6470); EFHC2, Ins, A_11, KKKSITQQRHQKAVQVLQVTFV, 22, 10.89, % positive cancer, 5.37, % positive normal, SEQ ID NO: 6471); SH2D2A, Del, C_11, PPPSPPIPSTRSPTSPSPSTPWGGAAPGKPRATSMRRWRWRSRLGPGGPPASSGTGPCGNAGPGLSRE ARIQEPGDHSLRTL, 82, 6.88, % positive cancer, 7.85, % positive normal, SEQ ID NO: 6472); ST6GALNAC5, Del, C_11, PPPGPRPRPPPRRGRWTDTSA, 21, 5.73, % positive cancer, 0.83, % positive normal, SEQ ID NO: 6473); ZBTB7C, Del, C_11, PPPHHPPSPATSSRTCSPTSLGGRWAPSRPRVTTVPISTS, 40, 8.88, % positive cancer, 7.02, % positive normal, SEQ ID NO: 6474); SH2D2A, Ins, C_11, PPPQALPSHLPGARRAHRLLRHGAGQPRGSPVQRLCGGGGGGPAWGRGAPLRPRAPGPAEMPVQA CPGKPESRSLATTV, 79, 10.03, % positive cancer, 5.79, % positive normal, SEQ ID NO: 6475); ST6GALNAC5, Ins, C_11, PPPPGPGPGPRRAAAAGRIPRREGPQAPENALQGLCPGDQLGAPAAQPARPPDRPDRVRHPHE, 63, 5.44, % positive cancer, 2.89, % positive normal, SEQ ID NO: 6476); ZBTB7C, Ins, C_11, PPPPTTPLPQRLLQGHVPRPPWGAAGPHQGRE, 32, 0.29, % positive cancer, 0.83, % positive normal, SEQ ID NO: 6477); LOC100685098, Del, G_11, GGGSPRGPQRPQPRPDGAGETLPALPPPSSPAPAAPAAAQTPPAGPRREPGAEPAAPRSGGGRARGR AAPASSPPTPDYRTPAAASRHRVGAPRGRVAVSSVAVGALPRGLGARRPGAPSGHSLRTRCPRRRP GGGWGPHRDGPSPSSCSHFSPCCWKSTPSWPELPLARAPEPLQPPAQAPSEPGALGAS, 191, 11.75, positive cancer, 9.09, % positive normal, SEQ ID NO: 6478); LOC480571, Del, G_11, GGGQRERERSKPGSWITRTSRPGCFCV, 27, 1.43, % positive cancer, 2.48, % positive normal, SEQ ID NO: 6479); MARK4, Del, G_11, GGGAGVCRMGPPPLPRWPTRPPPCPLGGPAPPPTSSPS, 38, 8.31, % positive cancer, 4.13, % positive normal, SEQ ID NO: 6480); OTUB2, Del, G_11, GGGSPSSPGTPCKARGAPRGAPWTPGAGTQPGLRAPRPWMRSPPVAPTAEPAGGSRGALPHRIAAR CGGTWPDGCGHYALQSPASGPGSLSLGWNGSRLQLGCNMDFGRVKHLST, 115, 13.47, % positive cancer, 9.5, % positive normal, SEQ ID NO: 6481); LOC100685098, Ins, G_11, GGGGLLGARSARSRVQTEPGKLCPRCPRPRPRLPRLPRRRRLLPPGRGGSRGPSPQPHGAGEDAHEE GRPPRARPPPPTTGRQPPRRATGSGPRGGEWR, 99, 12.61, % positive cancer, 12.4, % positive normal, SEQ ID NO: 6482); LOC480571, Ins, G_11, GGGVKEKGKGASREAGLQEHRDQAVSVCEELCLGDKIRPRACRQGVRSVGHGPFTGMARQFCP, 63, 1.15, % positive cancer, 2.48, % positive normal, SEQ ID NO: 6483); MARK4, Ins, G_11, GGGGRGCAEWAPRLSHAGPRGHPPAHWAAPPHHQPLHQADLQTDQKGHRRT, 51, 9.17, % positive cancer, 6.61, % positive normal, SEQ ID NO: 6484); OTUB2, Ins, G_11, GGGGPRPPRGRPARPEARPAVPHGRPARGPSLGSGPRVRGCARRPWPQRRSQPVAPAAPSRTGSLP AAAGPGPTAAATMPSRALPLGQEAYLWVGMDPGSSWAAIWTLAE, 110, 13.18, % positive cancer, 9.92, % positive normal, SEQ ID NO: 6485); SLC44A4, Del, U_11, FFFSPVASRGWVDTLKTPPSTTTGCPSWSPSWGPMSSPAASSVFLACVWTHSSSVSWKTWRGTTAR RTGPTTCPKPFSRS, 80, 8.31, % positive cancer, 9.5, % positive normal, SEQ ID NO: 6486); DCLK2, Del, C_12, PPPPAPHGRWSVVLSPGEAAGSLAGPRGRAPRGRAPPAPLPQPA, 44, 9.17, % positive cancer, 4.96, % positive normal, SEQ ID NO: 6487); WNT2B, Del, C_12, PPPPDGDRAAPAAAGLWVVGGAGLHLRRLPTAAGQLPRPPPLLPHPRSSRAPAPPAPPLLPHPPVLL PHPPAPPAPPSSRTPLLPHSPRSSRTPLLPHPPCSSRTPPAPPAPPSSRTPAPPAPRLLLPHPPSPPRPAPQ ARSAPPHPSPTRAARPAALTGCSWRPGSLIQ, 170, 16.05, % positive cancer, 10.33, % positive normal, SEQ ID NO: 6488); DCLK2, Ins, C_12, PPPPQLRTAAGPSFFPLGRLPVPWQAPEAGPPEAGLPPLPSPSQPELAAL, 50, 9.74, % positive cancer, 4.55, % positive normal, SEQ ID NO: 6489); LOC106558478, Ins, C_12, PPPPAVPRGVSAPSCPPRAPLTLEAAPGAGSAQRPPALKSPRSGRRCASPGGRADRLHAQRGARRG ARTPNPEAEAGLRGAAWTLPATW, 89, 9.46, % positive cancer, 7.02, % positive normal, SEQ ID NO: 6490); WNT2B, Ins, C_12, PPPPQMGTGLLPRPPGSGSSEGRASTCGVCPLRRDSSRAPPRSSRTPAPPAPPLLPHPRSSRTPPCSSRT PLLLPHPPPPAPPSSRTPPAPPAPPSSRTPPAPPAPPLLLPHPPPPAPPLLPHPACSSRTPPPRPAPPRRPA APRRIPPLREPPGPQR, 158, 8.31, % positive cancer, 7.02, % positive normal, SEQ ID NO: 6491); C20H19orf24, Del, G_12, GGGPGRGPSLRVGGPASPRLAARRLGSAGPGTPRRPPGPRGPAARGGAGAAPGSRARRARRARAG WDGPRAGRTPTPALSSPRPACRLKKPQRRRYGLLANTDDPTDMASLDSDEEIVFETRNLR, 125, 9.46, % positive cancer, 8.68, % positive normal, SEQ ID NO: 6492); LOC106559221, Del, G_12, GGGSPARAVGMPGAPRVPRRARSFRGHCCCWGWWPPSAWA, 40, 4.87, % positive cancer, 4.13, % positive normal, SEQ ID NO: 6493); C20H19orf24, Ins, G_12, GGGGPGAAPPSGSAAPRHPAWPRAASVLRGPGLPAGRQGPGVPPLGAGPERRRGRGRGGRGGRGR AGTARGLAGPRRRP, 79, 6.88, % positive cancer, 9.92, % positive normal, SEQ ID NO: 6494); LOC106559221, Ins, G_12, GGGGPRRELWGCQGRLGSPDGRALSGVTAAAGAGGRHLPGPEPHFPRGLRGLRVLLQTGRGRAD QAAQPVLCHLDGGGGRAHLLCCGGCRFLWK, 94, 6.02, % positive cancer, 3.72, % positive normal, SEQ ID NO: 6495); SYCP2, Del, A_13, KKKNLCYLVFCTISVEIKCPPNGHAGHL, 28, 3.72, % positive cancer, 1.65, % positive normal, SEQ ID NO: 6496); BMPER, Del, C_13, PPPPPLPGARLPPARTVSRLAG, 22, 4.87, % positive cancer, 1.24, % positive normal, SEQ ID NO: 6497); LOC478701, Del, C_13, PPPPPPRGPCSKDREGTQGPPSSLASRVPAPSLTCVSFSPFGRSSVTSTGSTPPAVTMETATCSWREST CTTMKLQVTNTCLGPSWWIWSRAPWTRSGPGPLARSSGPTTSCSARAVPGTTGPRATTPRAPSW, 133, 8.88, % positive cancer, 7.85, % positive normal, SEQ ID NO: 6498); BMPER, Ins, C_13, PPPPRRCRAPASLLRGLSPDSPAESPFRLRAAVRSRGGRTGPAGARDSLQGKNSVLRRQGSG, 62, 1.43, % positive cancer, 3.72, % positive normal, SEQ ID NO: 6499); LOC478701, Ins, C_13, PPPPRPHGAPAPRTGRGPRGRPPHWHPGCQPPP, 33, 2.58, % positive cancer, 3.31, % positive normal, SEQ ID NO: 6500); RASA1, Del, G_13, GGGGRGFPPVSCLPPRRAGAGPGSHTFS, 28, 1.43, % positive cancer, 1.24, % positive normal, SEQ ID NO: 6501); POU3F2, Del, G_13, GGGEPLRIRTQPFPAAAAAGFFHLGISEFSEQRGGGGAGAGQRGAAGPGGTRLARSPCAPEAPGEPR ARRGELRAAVARRRSGPPPSPSAPGRGASGASRRVCALRG, 107, 16.33, % positive cancer, 8.26, % positive normal, SEQ ID NO: 6502); TNFRSF8, Del, G_13, GGGGHGHLLHPDAGRGLLLEASFVVIRSRGRPRSSGAGEKHAARPAAPCTGPCPWLSGGGDCHSA TSRQGGRCRAAGRAPATATQSSRPARQTRGGSAGAARCRAWKGRGRWVLAHVLVRVSAARHVH AGKLQWART, 137, 6.88, % positive cancer, 8.68, % positive normal, SEQ ID NO: 6503); RASA1, Ins, G_13, GGGGGGGSRRFPASPRAAPGRGREATPSPSSEP, 33, 0.29, % positive cancer, 1.24, % positive normal, SEQ ID NO: 6504); TNFRSF8, Ins, G_13, GGGGATGTCCTQTLAEGCSSRHRSW, 25, 3.72, % positive cancer, 2.48, % positive normal, SEQ ID NO: 6505); ZNF354B, Del, C_14, PPPPSSGSGSSPLPLLPDLRVARPPPRFSQGSAGATPAPATPRAPARHPLPPPRATQQKGSASARSSFS KWPLRLLTSYPVSWERPRSAPHGDRPTVSRLQLLAGLAFPGITTGLKRTRIRREQNRSRTCRSALGG, 136, 9.46, % positive cancer, 8.26, % positive normal, SEQ ID NO: 6506); ZNF354B, Ins, C_14, PPPPPPRAPALPRSLSSPTSGSLDLRPASPRALRAPPRPLPHPVPLPGTLSPRPGPHSKKGLRPRGPPSA SGLCGFSPLTPSPGSAPDPLPTATVRLYPAFNF, 103, 18.34, % positive cancer, 9.5, % positive normal, SEQ ID NO: 6507); AKNAD1, Del, G_14, GGGGDSAGSGRGANRCGKSPPPRFPASCRCALRGCERRRCGRDQSLAHFPWRGQGSGGRAAPPQS SRTEWALVGAGRKTWRRKGTEGPTAESVPLPFKKRRCVQILLSVLIPAPAAILLPGLDCRVINVKTM ALRITIPEESVAKSHSKNFIIGTTAQDRIT, 162, 13.47, % positive cancer, 9.92, % positive normal, SEQ ID NO: 6508); RCBTB2, Del, G_14, GGGGTKGGALPSSLEVTPGPR, 21, 4.3, % positive cancer, 4.55, % positive normal, SEQ ID NO: 6509); AKNAD1, Ins, G_14, GGGGGTPRGRGGGRTGAGSRRLPDSRHPAAVP, 32, 3.72, % positive cancer, 3.31, % positive normal, SEQ ID NO: 6510); CCBE1, Del, C_15, PPPPRPGTRARRPRRPRARPSAPPRPPRQVAAAPVVLGSSARRTRSPRPGTRV, 53, 6.59, % positive cancer, 4.13, % positive normal, SEQ ID NO: 6511); CYP2U1, Del, C_15, PPPPRPPRRVRLPHLQTLRFASPSPSCLWAGEGVPSSSASASGRGDARRGRAGRVTQGTPGARGTRQ PIGVASAAARRPSPAPRPPRTAAAGLRAPPGPSRAQLIGGAGRGRGRSQSGAPRPPPPPPSPFGLWGA W, 136, 12.32, % positive cancer, 9.09, % positive normal, SEQ ID NO: 6512); MIER2, Del, C_15, PPPPRRPGPRPRARSPAPCPLPPGPANFAECAPRARGRDPGRDSNSGPP, 49, 6.59, % positive cancer, 5.37, % positive normal, SEQ ID NO: 6513); SYT2, Del, C_15, PPPPAVSTKVLGPPAKSFSRGGGRSPRIWGDASAAGAGRVRGWRGCRPVPGLPSWAELG, 59, 11.46, % positive cancer, 9.5, % positive normal, SEQ ID NO: 6514); CCBE1, Ins, C_15, PPPPPARGPAPADPAALAPGPPLPRGRPAKWPPRLSS, 37, 4.3, % positive cancer, 3.31, % positive normal, SEQ ID NO: 6515); CYP2U1, Ins, C_15, PPPPPVLPAASASRTCKLCALHPRRPPASGREKGSLPPPHPPLAAGTLGAGVQGESLRGHPGHEEPGS QSVSPAPRPDARAPRPARRGPQLRGCERRPGPLGRS, 104, 11.46, % positive cancer, 11.16, % positive normal, SEQ ID NO: 6516); SYT2, Ins, C_15, PPPPPPCPPRSWVPLRRASVGAEDEAPEFGGTRVRQGRGE, 40, 0.86, % positive cancer, 2.89, % positive normal, SEQ ID NO: 6517); WNT2B, Del, G_15, GGGGGRRAERRRETRLRGPHPRSGTPTLACGPGEPPSCF, 39, 5.73, % positive cancer, 5.37, % positive normal, SEQ ID NO: 6518); WNT2B, Ins, G_15, GGGGGDGGRKGAGRHGSGAPTLAAGPPPWPAVRESRPAVSDSPVLLARRG, 50, 9.17, % positive cancer, 4.13, % positive normal, SEQ ID NO: 6519); SIX5, Ins, C_16, PPPPPTPPPCCPPAPPSPPPSSPAGPRPRGAAPGVCGRGRGG, 42, 2.01, % positive cancer, 0.83, % positive normal, SEQ ID NO: 6520); KRT73, Del, A_18, KKKKKRKRSPFSPCPPRPAFPPRLLI, 26, 5.73, % positive cancer, 2.07, % positive normal, SEQ ID NO: 6521); STOML1, Del, G_18, GGGGGGRGTFPEPFSARRGTRGYPRGNLTVFRNPGFAFWGPKKGGCPPNGGARGPGPMRPRAGPP TSATASSASWGSCCCCSPSPFPAGSP, 91, 7.45, % positive cancer, 4.96, % positive normal, SEQ ID NO: and 6522); STOML1, Ins, G_18, GGGGGGAGAPSRSHSPQGGGPGATPGGI, 28, 1.72, % positive cancer, 2.07, % positive normal, SEQ ID NO: 6523).

In some embodiments, the array is a wafer-based, photolithographic, in situ peptide array produced using reusable masks and automation to obtain arrays of scalable numbers of combinatorial sequence peptides. In some embodiments, the peptide array comprises about 100, about 500, about 1000, about 2000, about 3000, about 4000, about 5,000, about 6000, about 7000, about 8000, about 9000, about 10,000, about 15,000, about 20,000, about 30,000, about 40,000, about 50,000, about 100,000, about 200,000, about 300,000, about 400,000, about 500,000, or more peptides having different sequences. Multiple copies of each of the different sequence peptides can be situated on the wafer at addressable locations known as features.

In some embodiments, the array is a glass slide or nitrocellulose membrane having in vitro synthesized peptides spotted in a predetermined pattern and screened for binding of antibodies in a biological sample from a patient.

In some embodiments, detection of antibody binding on a peptide array poses some challenges that can be addressed by the technologies disclosed herein. Accordingly, in some embodiments, the arrays and methods disclosed herein utilize specific coatings and functional group densities on the surface of the array that can tune the desired properties necessary for performing assays. For example, non-specific antibody binding on a peptide array may be minimized by coating the silicon surface with a moderately hydrophilic monolayer polyethylene glycol (PEG), polyvinyl alcohol, carboxymethyl dextran, and combinations thereof. In some embodiments, the hydrophilic monolayer is homogeneous. Second, synthesized peptides are linked to the silicon surface using a spacer that moves the peptide away from the surface so that the peptide is presented to the antibody in an unhindered orientation.

Platforms herein are also contemplated to include peptides in microtiter plates for determining T cell activity in response to FS peptides herein. In some embodiments, microtiter plates include but are not limited to 96 well, 384 well, 1536 well, 3456 well, and 9600 well plates. In some embodiments, more than one peptide is present in each well of a microtiter plate, i.e., the peptides are pooled and individual peptides eliciting T cell activity are determined by deconvolution of the positive and negative wells in the T cell assay.

### Screening for Immunogenic Peptides

Optionally, it is useful to determine immunogenicity of a candidate frameshift (FS) peptide for use in a universal cancer vaccine. Immunogenicity, as used herein, refers to the ability of a substance, such as a peptide, to elicit an immune response, such as an antibody response or a T cell response, when administered to an individual, for example, in a vaccine formulation. In some embodiments, a peptide that reacts with an antibody or elicits T cell activity in a biological sample from an individual is not immunogenic when administered in a vaccine formulation. In some embodiments, a peptide that reacts with an antibody or elicits T cell activity in a biological sample from an individual is immunogenic when administered in a vaccine formulation. Immunogenicity is determined by methods of those of skill in the art including in animal model testing and using in silico prediction of immunogenicity. In silico immunogenicity prediction tools are available for free to the public, for example at the Immune Epitope Database and Analysis Resource (www.iedb.org).

Alternatively, mice, such as humanized mice, and mice transgenic for human HLA genes are used to determine the immunogenicity of a candidate FS peptide. The candidate FS peptide is administered to the transgenic mouse in a vaccine formulation. Response to the vaccine is determined using antibody assays and/or T cell assays described elsewhere herein. If the mouse is injected with or engineered to develop a tumor, the protectiveness of the antigen can be determined.

### Vaccine Compositions and Formulations

Universal cancer vaccines herein comprise one or more peptides determined from the list in SEQ ID NO: 1-6554. For example, universal cancer vaccines, in some embodiments, comprise 1, 2, 3, 4, 5, 6, 7, 8, 9, 10, 11, 12, 13, 14, 15, 16, 17, 18, 19, 20, or more FS peptides. In some cases, the FS antigens are determined to have immunoreactivity in samples from people or dogs with cancer or when administered to people or dogs as in a clinical trial.

In one embodiment the vaccine can consist of plasmids encoding the MS FS variants. DNA or Gene Vaccines consist of a plasmid with a promoter and appropriate transcription and translation control elements. The plasmids may also contain sequences that encode peptide or protein fusions to the FS peptide to enhance, for example, expression levels, intracellular targeting or proteasomal processing. For example, the LAMP sequence when fused to a FS peptide sequence will enhance MHCII responses. These plasmids can be introduced by a needle, a gene gun, an aerosol injector, with patches, via microneedles, by abrasion, orally, nasally among many forms. The plasmid can be introduced into the blood, the thymus, the pancreas, the skin, the muscle, mucus membrane, a tumor or other sites.

Alternatively or in combination, the MS FS peptides can be encoded in RNA that is directly introduced into the person or dog. The RNA can be chemically synthesized or more commonly in vitro transcribed. The RNA will encode one or more FS peptides and will include signals to enhance stability and translation. The RNA may also include unnatural nucleotides to increase the half-life. These RNAs can be introduced by a needle, a gene gun, an aerosol injector, with patches, via microneedles, by abrasion, orally, nasally among many forms. The RNA can be introduced into the blood, the thymus, the pancreas, the skin, the muscle, mucus membrane, a tumor or other sites.

In additional embodiments, the FS peptides coding sequences can be introduced into a virus as a vector. The FS sequences can be fused to other sequences that enhance transcription, translation or presentation to the immune system. These viral vectors include pox viruses, adenovirus, lentiviruses, retroviruses, alpha viruses and others using a needle, a gene gun, an aerosol injector, with patches, via microneedles, by abrasion among many forms. They may also be administered by inhalation or ingestion.

Alternatively, or in combination, the FS peptides can be administered via a bacterial vector. The FS coding sequences are introduced into the bacteria, usually in the form of plasmid or lysogenic phage, and the bacteria administered to the subject. Listeria is commonly used in this way, but other bacteria could be used or developed. The bacteria can be administered by needle to the blood or intraperitoneal injection. Bacteria can also be administered orally.

The FS peptides can also be delivered as peptides. Usually the peptides are 10 aa long or longer, preferably 25 aa or longer. 25-40 aa long may be ideal. Usually the peptides are fused to a carrier such as albumin, keyhole limpet protein etc. In some forms the peptide is incorporated into liposomes or other forms of nanobodies.

The vaccines may be incorporated into liposomes or various forms of nanoparticles. DNA, RNA, virus, bacteria or peptides can be incorporated into or onto the liposomes or nanoparticles.

Alternatively, vaccines herein are contemplated to be delivered to an individual pre-loaded into antigen presenting cells. In some instances, the MS FS neo-antigens as peptides or encoded as nucleic acids can be loaded into antigen presenting cells, such as dendritic cells or macrophages, and the loaded cells administered to the individual. In other cases, genes encoding the neo-antigens can be used to transform antigen presenting cells through techniques known by those of skill in the art such as CRISPER, liposome transfection, or viral transformation.

Universal vaccine formulations herein comprise a universal vaccine composition in a biologically acceptable formulation, gaseous, liquid or solid, or mixture thereof, which are suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

To increase an immune response and efficacy in treating cancer, FS peptides herein are optionally coupled to another protein such as ovalbumin or keyhole limpet hemocyanin (KLH), thyroglobulin or a toxin such as tetanus or cholera toxin. FS peptides herein are also mixed with adjuvants or checkpoint inhibitors.

Adjuvants include, for example: ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan.

Checkpoint inhibitors are inhibitors of proteins known to potentiate or inhibit an immune response and accordingly a checkpoint inhibitor increases the immune response. In some instances, tumors are known to upregulate checkpoint inhibitors to evade the immune response in a patient and checkpoint inhibitors allow the immune system of the patient to recognize and eliminate the tumor. Checkpoint inhibitors herein include but are not limited to a PD-1 inhibitor, a PD-L1 inhibitor, a CTLA-4 inhibitor, Pembrolizumab, Nivolumab, or Atezolizumab. Checkpoint inhibitors may also be administered in healthy people as adjuvants to the preventative vaccine.

Cosolvents may be added to a FS peptide composition or formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters.

Supplementary compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

Pharmaceutical compositions can be formulated to be compatible with a particular route of administration. Thus, pharmaceutical compositions include carriers, diluents, or excipients suitable for administration by various routes. Exemplary routes of administration for contact or in vivo delivery which a composition can optionally be formulated include inhalation, respiration, intranasal, intubation, intrapulmonary instillation, oral, buccal, intrapulmonary, intradermal, topical, dermal, parenteral, sublingual, subcutaneous, intravascular, intrathecal, intraarticular, intracavity, transdermal, iontophoretic, intraocular, opthalmic, optical, intravenous (i.v.), intramuscular, intraglandular, intraorgan, or intralymphatic.

Formulations suitable for parenteral administration include aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

### Methods of Eliciting an Immune Response

Also provided herein, are methods of eliciting an immune response in an individual, the methods comprising, a) preparing a vaccine composition comprising a population of peptides that are predicted and or demonstrated to have immunoreactivity; and b) administering the vaccine composition to an individual, wherein administering the vaccine composition elicits an immune response in the individual against a cancer. In some embodiments, the immune response protects the individual from developing cancer. In some embodiments, the immune response eliminates a pre-existing cancer from the individual.

### Methods of Treatment

Provided herein, in some aspects, are methods of treating an individual in need of treatment for a cancer using a universal cancer vaccine. Some such methods comprise, a) preparing a vaccine composition; and b) administering the vaccine composition to the individual, wherein administering the vaccine composition elicits an immune response in the individual against the cancer and wherein the cancer in the individual is reduced.

A universal therapeutic vaccine could also be administered to prevent reoccurrence in a person who has had cancer.

Methods herein comprise administration of a vaccine composition to an individual. Vaccine compositions herein, in some embodiments, comprise a pharmaceutically acceptable adjuvant or excipient. In some embodiments, the adjuvant is selected from the group consisting of ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan. Vaccine compositions herein, in some embodiments, are administered via a route selected from the group consisting of subcutaneous, intradermal, intramuscular, intranasal, intravenous, and sublingual. Individuals in need of administration of a universal vaccine, in some embodiments are mammals. In some embodiments, the individual is a human, a dog, a cat, a mouse, a rat, a rabbit, a horse, a cow, or a pig.

Methods herein comprise administration of a universal vaccine in an individual. In some embodiments, the individual is diagnosed with cancer. In some embodiments, the individual has increased risk of developing cancer. In some embodiments, the cancer comprises Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, or Wilms' tumor.

### Definitions

“Neo-antigens”, used herein, refers to polypeptides encoded by variants in tumor cells, such as MS frameshift variants.

“Frameshift variants”, used herein, refers to variants caused by insertion/deletion variants in the MS in RNA which cause a shift in the reading frame of an mRNA creating a peptide with variant amino acid residues at the C-terminus and an early termination.

“In/del” or “insertion/deletion”, used herein, refers to variants caused by insertion or deletion of one or two nucleotides in a MS in the RNA. In most cases, in/del variants result in a frameshift variant.

“Universal cancer vaccine”, used herein, refers to a cancer vaccine that is effective for multiple cancer types and multiple individuals.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: A Model as the Basis for Developing a Universal Cancer Vaccine

A model summarizing the basis for a general therapeutic or prophylactic cancer vaccine herein is presented in FIG. 3. As information flows from DNA to RNA to protein there is a general increase in error rate. These errors include the insertions and deletions (Indel) of one or more nucleotides during transcription in microsatellites (MS) in exons. These errors will produce a background level of frameshift (FS) transcripts, which encode truncated proteins with a FS peptide at the C-terminus. The level of the FS peptides in normal cells is managed by the protein quality control mechanisms, such as nonsense media decay (NMD) and ER-associated degradation. However, in an aberrant and potentially cancerous cell there is a global increase in these errors due to chromosomal instability or key, broadly effective variants. Under these conditions the number of FS peptides produced exceeds the protein quality control mechanisms allowing FS peptides to be presented in MHC I and II complexes or externally to dendritic cells. In most instances the aberrant cells are killed due to inherent dysfunction or by the immune system. However, some cells are able to evade these mechanisms by so by decreasing MHC I, II expression and/or establishing an immunosuppressive environment. An important aspect of the model is that because of the global increase in the error rate of transcription the FSs will be continually produced. Thus, bystander neo-antigens would be good immunological targets.

### Example 2: Demonstration of Indels in the RNA but not DNA

To determine whether common FS variants in different cancers are produced more by errors at RNA transcription rather than DNA variants, the MS region of Sec62 was examined in both human and dog tumors. There were no detectable insertions or deletions in the DNA for either MS. However, there was a detectable level of insertion of one A in the RNA of the same tumor for both MSs (FIG. 13).

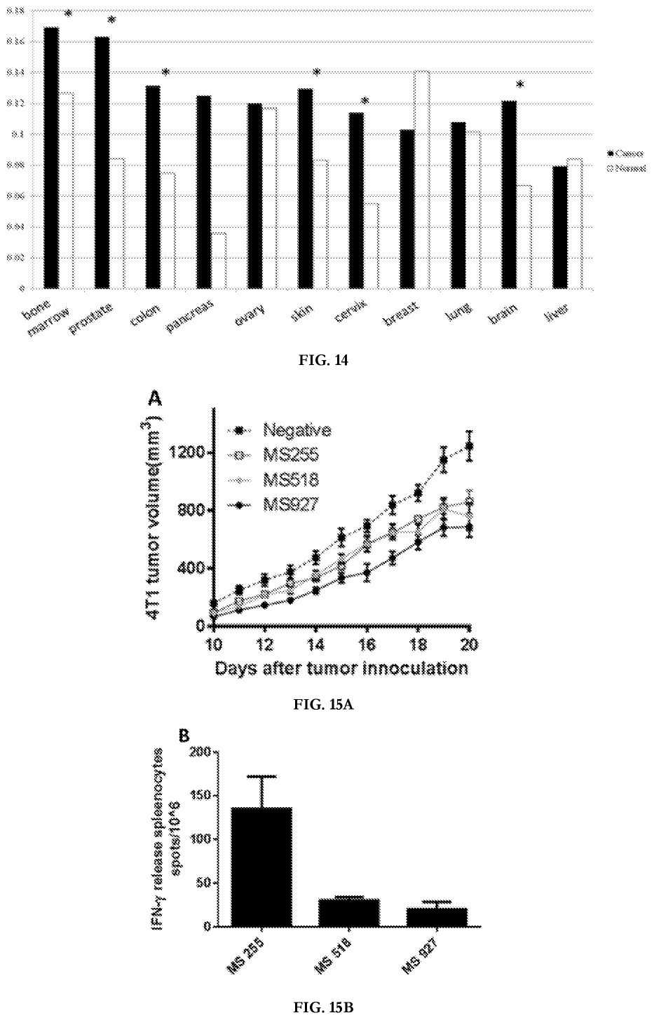

To determine whether tumor cells have more FS transcripts than normal cells, an MS Indel rate analysis with the current human EST database was performed and showed that Indel rate of homopolymer in coding region is significantly higher in different cancers than it in normal cell transcripts (FIG. 14).

### Example 3: Immunoreactivity of Frameshift Peptides

It was determined that the increase of aberrant proteins, including those with FSs, overwhelms the protein quality control system thereby eliciting immune responses to them. We developed an array displaying all possible MS FS encoded in the human proteome meeting length and expression criteria described earlier. There are ˜3,000 such peptides (and ˜220K from exon mis-splicing). In FIG. 4, FIG. 5, FIG. 6, and FIG. 7 we show that these arrays can be used to compose universal vaccines of different types. A universal vaccine for 6 late stage human tumors and 8 dog late stage tumors is shown. A different set of peptides is chosen for a universal protective vaccine.

### Example 4: Frameshift Peptide Vaccines Protect Against Cancer

To determine whether any MS Indel event could produce a FS in any constitutive gene and could therefore be a protective antigen against most or all tumor types, three predicted MS FSs in mouse genes were selected using the criteria of FS peptide length and predicted H2-D epitopes (Table 1). Each FS peptide was tested individually in the 4T1-BALB/c mouse breast tumor model with the prophylactic vaccination of gene vaccine/peptide boost. Each FS peptide vaccination significantly slowed the tumor growth compared to the mock immunized mice (FIG. 15A). Each FS antigen can elicit specific IFNγ releasing splenocytes (FIG. 15B). These results indicate that any FSs produced by MS can confer protection. Peptides tested are listed in Table 1.

TABLE 1
The three MS FSPs with predicted
H2-D epitopes for the vaccine test.

| Gene | MS | FS | FS | FS peptide |
|---|---|---|---|---|
| ID | type | type | size | (Kd/Ld score >20) |
| MS0518 | 10A | Del | 59 aa | TLCMEVMLRWNTR |
| | | | | E [symbol] NTHFLHTSQE |
| | | | | EK [symbol] TWTSRCGSF |
| | | | | VIRPL |
| MS7927 | 9A | Del | 33 aa | ICM [symbol] QAPETTSA |
| | | | | ACKASYRPEGLYL |
| MS2255 | 9A | Ins | 25 aa | VFSCDKRCIKHTA |
| | | | | GNKS [symbol] TFSGY |

[symbol]

indicates data missing or illegible when filed

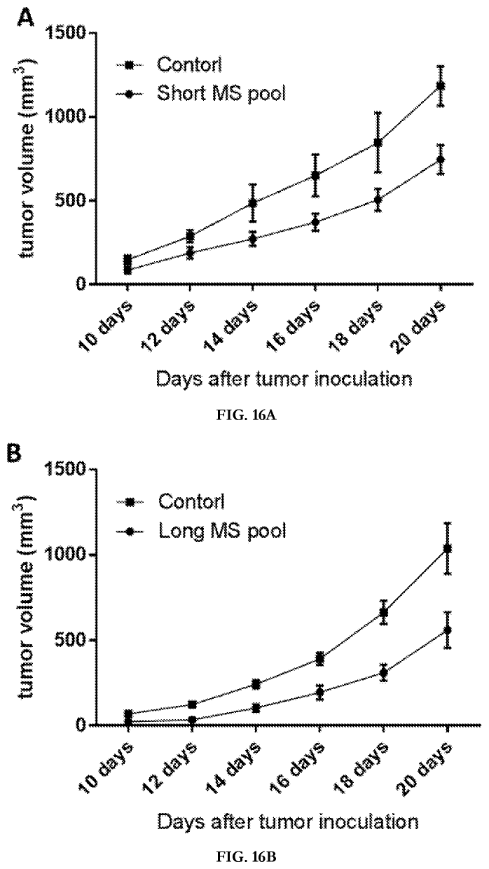

In another demonstration, two different pools of mouse FS antigen of the different MS length were selected (Table 2) and tested in the same mouse tumor model. Both FS pooled vaccine significantly retarded tumor growth comparing to the control group (FIG. 16A and FIG. 16B).

TABLE 2
Two pools of the FS antigens from the different MS length.

| | | | | |
|---|---|---|---|---|
| | | MS | | peptide size |
| Ag ID | Ref Gene | type | INDEL | (aa) |

Short MS pool

| | | | | |
|---|---|---|---|---|
| Mus385 | NM_146792.2 | 8T | Deletion | 32 |
| Mus392 | NM_029998.3 | 8T | Deletion | 51 |
| Mus023 | NM_147003.1 | 8T | Insertion | 19 |
| Mus263 | NM_001114663.1 | 8A | Insertion | 20 |
| Mus942 | NM_001081355.3 | 8A | Insertion | 37 |
| Mus671 | NM_001163189.1 | 8A | Insertion | 49 |
| Mus334 | NM_130448.3 | 8A | Deletion | 29 |
| Mus051 | NM_033618.3 | 8A | Insertion | 26 |
| Mus587 | NM_001081302.1 | 8A | Insertion | 32 |
| MS255 | NM_010086.4 | 9A | Insertion | 24 |
| MS927 | NM_053009.3 | 9A | Deletion | 33 |
| MS518 | NM_153511.3 | 10A | Deletion | 59 |

Long MS pool

| | | | | |
|---|---|---|---|---|
| Mus487 | NM_001109759.1 | 10A | Deletion | 44 |
| Mus413 | NM_027009.2 | 10A | Deletion | 27 |
| Mus274 | NM_001081345.2 | 10A | Insertion | 14 |
| Mus694 | NM_028787.4 | 10T | Insertion | 45 |
| Mus281 | NM_001160399.1 | 11A | Deletion | 39 |
| Mus951 | NM_025441.3 | 11A | Deletion | 36 |
| Mus461 | NM_001033226.4 | 13T | Deletion | 22 |
| Mus528 | NM_001034881.3 | 14T | Deletion | 54 |
| Mus414 | NM_028664.1 | 18T | Deletion | 28 |

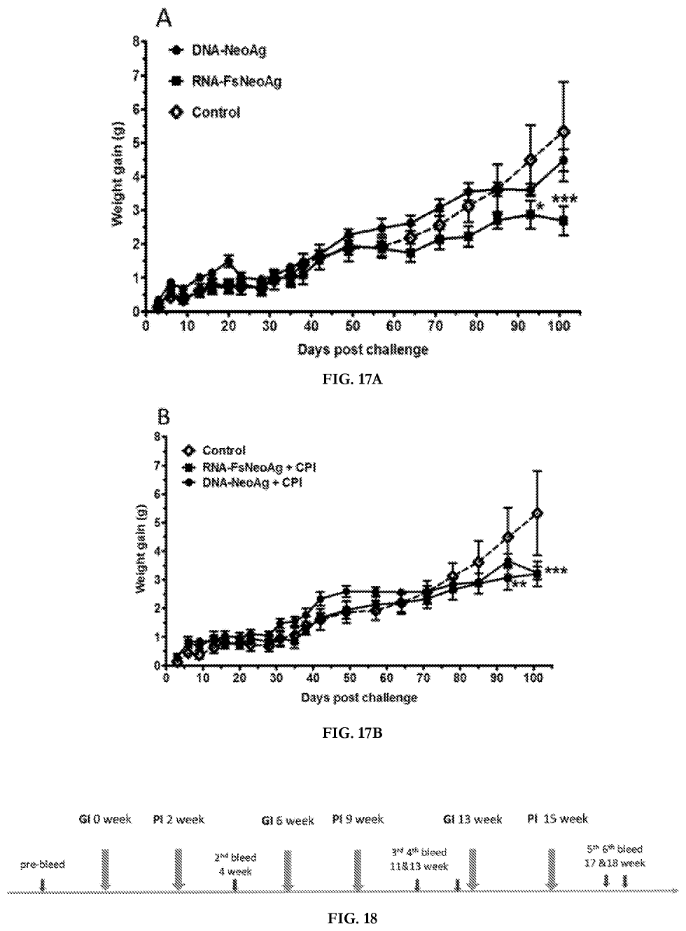

We also tested the FS vaccine in a mouse ovarian cancer model (ID8-057BL6). A pool of FSP from 10 MS FS and 3 mis-splicing FS was immunized, as well as a pool of peptides from the point DNA mutation if the ID8 cells were immunized the mice after the tumor cell injection. Both vaccine showed the significant protection comparing to the control mice which were immunized with PBS (FIG. 17). The same sets of the vaccines combined with immune checkpoint treatment also showed the significant protection. This experiment shows our FS variants have the same protection as the neo-antigens from the DNA mutations.

### Example 5: Reactivity on the Arrays Corresponds to Protection

As shown in FIG. 9 there are peptides that are not observed to be positive in any cancer samples in the assay but are sporadically positive in non-cancer individuals. We identified mouse homologs of these “negative” peptides and tested whether they protected in 2 different mouse models. The negative FS did not offer protection and the positive peptides did. In another example, the blood from individual mice was screened on the arrays. Positive and negative peptides were chosen for each mouse and tested as vaccines. As can be seen in FIG. 10, the positive peptides were much more protective. From these examples we conclude that the arrays can be used to predict effectiveness as vaccines both positive and negative.

### Example 6: Clinical Trial for a Universal Cancer Vaccine

Ten individuals presenting with histologically confirmed stage 1 breast cancer are in need of treatment. Each individual is given a vaccine comprising 20 MS FS antigens after receiving lumpectomy surgery. The vaccine is designed by screening 100 women with stage 1 breast cancer on the FS peptide arrays for FS that are recurrently positive on the arrays. Vaccinated individuals are monitored for breast cancer recurrence and nine of the ten individuals remain cancer free for five years. Ten individuals receive a lumpectomy surgery only and six of the ten individuals remain cancer free for five years. This Example demonstrates that a universal therapeutic cancer vaccine provides increased efficacy in breast cancer treatment compared to lumpectomy surgery only.

### Example 6: Clinical Trial for a Universal, Broadly Preventative Cancer Vaccine

One hundred thousand individuals not having cancer but over 60 years old are enrolled in a vaccine trial. Half are given a vaccine comprising 30 MS FS antigens. These antigens are chosen by screening the blood of 100 people who have stage 1 cancer for each of the 8 most prevalent human cancers on the FS peptide arrays. The most common reactive peptides across all cancers are chosen for the vaccine. Half are given a mock vaccine. Vaccinated individuals are monitored for any cancer. The expected incidence rate of cancer in this group is 5% per year. This is the incidence observed on average in the control group over 5 years of the trial. The incidence of cancer in the vaccinated group is only 0.5% on average over 5 years. This is deemed significant protection. This Example demonstrates that a universal cancer vaccine provides protection against major cancer broadly in the population.

### Example 7: Clinical Trial of a Vaccine to Prevent Reoccurrence of Cancer

Dogs that have been treated with standard of care for the particular cancer are enrolled in a trial. Dogs with any cancer are eligible. 100 dogs are enrolled. After standard treatment, one half of the dogs receive a MS, FS vaccine of 20 FS antigens. The other half receives a mock vaccination. It is a blinded trial. Data is obtained on length of survival and other treatments of the dogs. At the end of one year the trial is unblended. There is a significant survival advantage in the dogs receiving the vaccine.

### Example 8: Diagnosis of Cancer Using MS FS Arrays

A large cohort of people is enrolled in a diagnostic trial. Individuals provide blood samples every 6 months. These samples are assayed on arrays of 5000 MS FS peptides. A significant increase in the number of FS peptides reactive with the sera in successive samples is an indication of cancer. Over 3 years the diagnostic was able to predict incidence of cancer before standard diagnosis in 90% of the events.

### Example 9: Methods for a Universal Cancer Vaccine

The Microsatellite (MS) FS Peptide Database and Analysis

The human whole mRNA coding sequence was acquired from the NCBI CCDS database. The dog whole mRNA coding sequence was acquired from the NCBI RefSeq database. The coding sequence with the MS region (>6 nucleotides homopolymer) were analyzed. The FS peptide caused by the possible insertion or deletion of the each MS during the transcription were summarized in the database.

The essential gene database was summarized from the essential gene database (found at essentialgene.org). Contain 2294 genes form 17 database of 7 studies. It covers 7 cancer sand one normal. The high expression gene database was summarized from the top 20% genes of the average expression level in 11 normal tissues. The data of the gene expression was form the RNA Seq Atlas (found at medicalgenomics.org/rna_seq_atlas). Cancer driver gene list was summarized from the database of COSMIC (found at cancer.sanger.ac.uk/census).

TABLE 3
The criteria for vaccine candidate selection from database
of the MS INDEL. The minimum homopolymer we have tested
is 7. The T cell epitope is from 8 a.a. And the average
protein functional domain is 100 a.a.

| | |
|---|---|
| Homopolymer size (nt) | >6 |
| FS peptide size (a.a) | 8 to 100 |
| Essential Gene | any one |
| Top20% Expression in normal | |
| Cancer Driver Gene | |

FSP Array Analysis

The peptide sequence of the FSP array was from our human or dog FS database. It includes the most of the MS FS antigens (homopolymer size >6 nt, peptide size >9 a.a) and potential FSP caused by mis-splicing of each exon. All FS antigens were segmented into 10 to 15 a.a long peptides. The array was synthesized by Nimblegen with photolithography. There were about 400K peptides of each FSP array, including ˜14K MS FSPs. We also developed small FSP arrays for human and dog with about 800 FSPs on each array. The 17 a.a FSPs were synthesized by Sigma, China Peptides Co. and Watsonbio Sciences. The array was printed on the NSB9 Amine Slides (AMIN25, NSB POSTECH, Inc.) by the Applied Microaarays.

Serum was diluted 1:100 or 1:200 in the dilution buffer and incubated with array over night at room temperature or 4° C. After washing, the IgG was detected by the labeled secondary antibody. The RFU of each peptide was detected by the scan. The raw RFU was processed and normalized for the analysis. The positive cut-off value of each peptide is calculated by average plus two fold of the standard deviation of the control samples of that peptide.

### Mouse Protection Analysis

The mouse vaccine antigens were selected from the mouse MS FS database for the protection analysis. The full length of the mouse MS FS peptide sequences were cloned into the pCMVi genetic vaccine vectors for the genetic immunization. The overlapping peptides were synthesized and conjugated to the KLH protein for the peptide immunization. The control group was immunized with the empty pCMVi vectors and KLH protein. The CpG 2216 (synthesized by Invitrogen) and the pCMVi-LTA and pCMVi-LTB were used as the adjuvant for the genetic immunization and poly:IC (Cat # P1530, Sigma). Alternatively, CpG plus Alum was used as the adjuvant for the peptide immunization.

In general, the 8 weeks old BALB/c mice were immunized with 1 μg pooled antigen plasmids and 0.5 μg CpG 2216 and 0.5 μg LTA, LTB-pCMVi plasmids and boosted in two to three weeks with the same dosage. The mice were also immunized with 50 μg pooled of FS peptides conjugated KLH with 50 μg CpG2216 and Alum or 50 μg Poly:IC two to three weeks after the genetic immunization.

Two weeks after the final immunization, mice were challenged of 5×10³ 4T1 cells by subcutaneous injection. Tumor size was measured and immune response was analyzed by the end of the experiment.

EST Database Analysis

The EST database was downloaded from the NCBI (December, 2010 version). EST sequences were aligned with human mRNA Reference Sequences by BLASTN program. EST sequences that aligned with a single reference sequence or single loci were selected based on similarity and length of alignments and their origin. The similarity and length of alignments selected was ≥50 bp with 97% similarity, ≥85 bp with 95% similarity or ≥100 bp with 90% similarity. In addition, the selected loci were located unambiguously in single reference sequence or loci. The origin of the EST was unequivocal when there was only one alignment with a reference sequences or the BLAST score of the best alignment was higher than that of second best alignment by at least 50. Non-coding RNA was excluded from second best alignment with the only exception when they had the exact same BLAST score with the best one. After all applied criteria, EST sequences were selected that had the best alignments only from a single gene and used this database for the MS INDEL analysis. The INDEL EST sequences were then detected by the sequence analysis. The INDEL rate was calculated by the percentage of the accounted INDEL sequences of the all MS sequences in each study. The chi-square test was used to assess statistically significance.

Sequence Analysis of the SEC62 Gene

Genomic DNA and total RNA were extracted from the four human breast cancer cell lines (HTB26, CRL2315, CRL1504, CRL2326), five dog mast cell tumors and three dog carcinomas from the collaborator in Colorado State University. The primers were designed and synthesized to amplify the MS region of the SEC62 gene in cDNA and genomic DNA of both human and dog. The PCR products were sequenced and the sequence traces were analyzed to detect the INDEL of the MS region. The human genomic SEC62 primers: forward: 5′-TGCCATACCTGTTTTTCCC-3′; reverse: 5′-AGTTATCTCAGGTAGGTGTTGC-3′. The human cDNA SEC62 primers: 5′-AAAGGAAAAGCTGAAAGTGGAA-3′; reverse: 5′-GCAACAGCAAGGAGAAGAATAC-3′. Dog genomic primers: forward: 5′-AAGGGAGTCTGTGGTTGA-3′; reverse: 5′-CAAAGAGGGAAGAGAGTGG-3′. The human cDNA SEC62 primers: 5′-AAGGGAGTCTGTGGTTGA-3′; reverse: 5′-CAAAGAGGGAAGAGAGTGG-3′.

Safety Trial of the Dog Prophylactic Cancer Vaccine

Total 21 FS antigens were selected and synthesized in both versions of the plasmid and KLH conjugated overlapping peptides. Five of them were caused by the mis-splicing and 16 were caused by the INDEL of the MS transcription. Both genetic immunization (GI) and the peptide immunization (PI) were delivered intradermally. For the GI, DNA fragments encoding each antigen were cloned into the plasmids (PCMVi-UB and PCMVi-LS-rCOM-TT) and the two plasmids were mixed equally as an antigen composition for the GI. Each antigen composition was also equally mixed. One GI is composed by 300m mixed antigens and 300m Poly:IC in 500 ul PBS. For the PI, overlapping peptides were synthesized for each antigen. Synthesized peptides for the same antigen were equally mixed as a pool and conjugated with KLH 1:1 by weight as an antigen composition for the PI. Each peptide conjugated KLH were equally mixed for each vaccine. One PI is composed by 300m mixed peptide-KLH and 300m Poly:IC in 500 ul PBS.

Each dog was immunized with two vaccines with four weeks apart (FIG. 18). Two phases of each vaccine are applied two weeks apart.

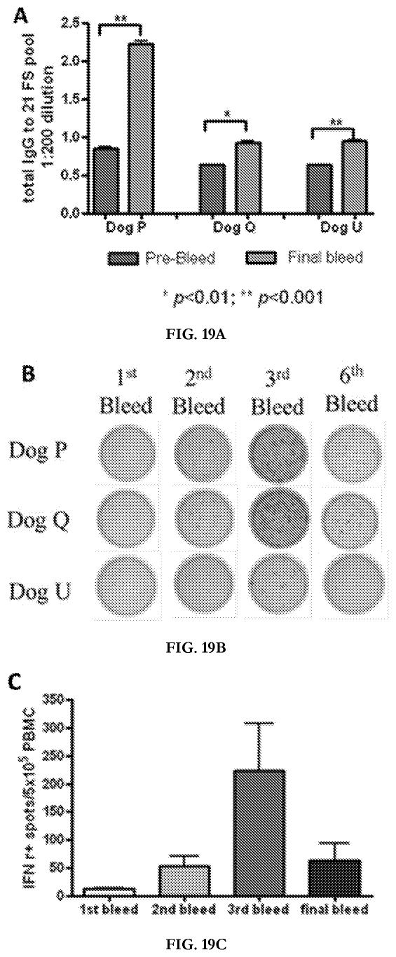

The antibody response to the vaccine antigens were analyzed by the ELISA (FIG. 19A) and the T cell response were analyzed by the IFN-r released PBMC with the kit from the R&D system (FIG. 19B and FIG. 19C) (Cat # SEL781 and SEL002). The dog pathological analysis showed the vaccine did not induce any related auto immune disease.

While preferred embodiments have been shown and described herein, such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a cancer in an individual, the method comprising: administering one or more peptide(s), wherein at least one peptide comprises a variant in a microsatellite (MS) coding region of an expressed gene, wherein the one or more peptide(s) are at least 8 amino acids in length and wherein administration of the peptide(s) or nucleic acid(s) encoding the one or more peptide(s) results in an immune response to the cancer.

2. The method of claim 1, wherein the one or more peptide(s) is selected from the group consisting of peptides in SEQ ID NO: 1-6554.

3. The method of claim 1, wherein the immune response to the cancer is an antibody response.

4. The method of claim 1, wherein the immune response to the cancer is a T cell response.

5. The method of any one of claims 1 to 4, wherein treating the cancer comprises reducing tumor size, inhibiting tumor growth, reducing tumor burden, increasing survival, or increasing cancer-free survival.

6. The method of any one of claims 1 to 5, wherein the peptide(s) are encoded by an mRNA comprising a frameshift variant expressed by a cancer cell.

7. The method of claim 6, wherein the frameshift variant is created by a transcription insertion or deletion error in a microsatellite.

8. The method of claim 6 or claim 7, wherein the frameshift variant is downstream of a microsatellite.

9. The method of any one of claims 1 to 8, wherein the peptides comprise at least one T cell epitope.

10. The method of any one of claims 1 to 9, wherein the peptides comprise at least one B cell epitope.

11. The method of any one of claims 1 to 10, wherein the peptides are administered as a nucleic acid encoding the peptides.

12. The method of any one of claims 1 to 11, wherein the peptides are administered in a vaccine composition.

13. The method of claim 12, wherein the vaccine composition comprises an adjuvant.

14. The method of claim 13, wherein the adjuvant is selected from the group consisting of ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan.

15. The method of any one of claims 1 to 14, wherein the method further comprises administration of a checkpoint inhibitor.

16. The method of claim 15, wherein the immune checkpoint inhibitor is selected from the group consisting of a PD-1 inhibitor, a PD-L1 inhibitor, and a CTLA-4 inhibitor.

17. The method of claim 15 or claim 16, wherein the immune checkpoint inhibitor is selected from one or more of the group consisting of Pembrolizumab, Nivolumab, and Atezolizumab.

18. The method of any one of claims 1 to 17, wherein peptides are administered via a route selected from the group consisting of subcutaneous, oral, mucosal, intradermal, intramuscular, intranasal, intravenous, and sublingual.

19. The method of any one of claims 1 to 18, wherein the individual is a mammal.

20. The method of any one of claims 1 to 19, wherein the individual is a human, a dog, a cat, a mouse, a rat, a rabbit, a horse, a cow, or a pig.

21. The method of any one of claims 1 to 20, wherein the cancer is selected from the group consisting of Acute lymphoblastic leukemia, Acute monocytic leukemia, Acute myeloid leukemia, Acute promyelocytic leukemia, Adenocarcinoma, Adult T-cell leukemia, Astrocytoma, Bladder cancer, Bone Cancer, Brain Tumor, Breast Cancer, Burkitt's lymphoma, Carcinoma, Cervical Cancer, Chronic Lymphocytic Leukemia, Chronic myelogenous leukemia, Colon Cancer, Colorectal cancer, Endometrial cancer, Glioblastoma multiforme, Glioma, Hepatocellular carcinoma, Hodgkin's lymphoma, Inflammatory breast cancer, Kidney Cancer, Leukemia, Lung cancer, Lymphoma, Malignant Mesothelioma, Medulloblastoma, Melanoma, Multiple myeloma, Neuroblastoma, Non-Hodgkin Lymphoma, Non-Small Cell Lung Cancer, Ovarian Cancer, Pancreatic Cancer, Pituitary tumor, Prostate cancer, Retinoblastoma, Skin Cancer, Small Cell Lung Cancer, Squamous cell carcinoma, Stomach cancer, T-cell leukemia, T-cell lymphoma, Thyroid cancer, and Wilms' tumor.

22. A method of preventing cancer in an individual, the method comprising: administering one or more peptides selected from the group consisting of SEQ ID NO: 1-6554 or a nucleic acid encoding the peptide(s), wherein administration of the peptide(s) or the nucleic acid(s) results in an immune response to the cancer.

23. The method of claim 22, wherein the immune response to the cancer is an antibody response.

24. The method of claim 22, wherein the immune response to the cancer is a T cell response.

25. The method of any one of claims 22 to 24, wherein preventing the cancer comprises reducing cancer incidence.

26. The method of any one of claims 22 to 25, wherein the peptide(s) are encoded by an mRNA comprising a frameshift variant expressed by a cancer cell.

27. The method of claim 26, wherein the frameshifted variant is created in a transcription insertion or deletion error in a microsatellite.

28. The method of claim 26 or claim 27, wherein the frameshift variant is downstream of a microsatellite.

29. The method of any one of claims 22 to 28, wherein the peptides comprise at least one T cell epitope.

30. The method of any one of claims 22 to 29, wherein the peptides comprise at least one B cell epitope.

31. The method of any one of claims 22 to 30, wherein the peptides are administered as a nucleic acid encoding the peptides.

32. The method of any one of claims 22 to 31, wherein the peptides are administered in a vaccine composition.

33. The method of claim 32, wherein the vaccine composition comprises an adjuvant.

34. The method of claim 33, wherein the adjuvant is selected from the group consisting of ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan.

35. The method of any one of claims 22 to 34, wherein the method further comprises administration of a checkpoint inhibitor.

36. The method of claim 35, wherein the immune checkpoint inhibitor is selected from the group consisting of a PD-1 inhibitor, a PD-L1 inhibitor, and a CTLA-4 inhibitor.

37. The method of claim 35 or claim 36, wherein the immune checkpoint inhibitor is selected from one or more of the group consisting of Pembrolizumab, Nivolumab, and Atezolizumab.

38. The method of any one of claims 22 to 37, wherein peptides are administered via a route selected from the group consisting of subcutaneous, intradermal, intramuscular, intranasal, intravenous, and sublingual.

39. The method of any one of claims 22 to 38, wherein the individual is a mammal.

40. The method of any one of claims 22 to 39, wherein the individual is a human, a dog, a cat, a mouse, a rat, a rabbit, a horse, a cow, or a pig.

41. The method of any one of claims 22 to 40, wherein the cancer is selected from the group consisting of Acute lymphoblastic leukemia, Acute monocytic leukemia, Acute myeloid leukemia, Acute promyelocytic leukemia, Adenocarcinoma, Adult T-cell leukemia, Astrocytoma, Bladder cancer, Bone Cancer, Brain Tumor, Breast Cancer, Burkitt's lymphoma, Carcinoma, Cervical Cancer, Chronic Lymphocytic Leukemia, Chronic myelogenous leukemia, Colon Cancer, Colorectal cancer, Endometrial cancer, Glioblastoma multiforme, Glioma, Hepatocellular carcinoma, Hodgkin's lymphoma, Inflammatory breast cancer, Kidney Cancer, Leukemia, Lung cancer, Lymphoma, Malignant Mesothelioma, Medulloblastoma, Melanoma, Multiple myeloma, Neuroblastoma, Non-Hodgkin Lymphoma, Non-Small Cell Lung Cancer, Ovarian Cancer, Pancreatic Cancer, Pituitary tumor, Prostate cancer, Retinoblastoma, Skin Cancer, Small Cell Lung Cancer, Squamous cell carcinoma, Stomach cancer, T-cell leukemia, T-cell lymphoma, Thyroid cancer, and Wilms' tumor.

42. A method of eliciting an immune response in an individual comprising administering one or more peptides selected from the group consisting of SEQ ID NO: 1-6554 wherein administration of the peptide(s) results in an immune response to the peptides.

43. The method of claim 42, wherein the immune response to the cancer is an antibody response.

44. The method of claim 42, wherein the immune response to the cancer is a T cell response.

45. The method of any one of claims 42 to 44, wherein preventing the cancer comprises reducing cancer incidence.

46. The method of any one of claims 42 to 45, wherein the peptide(s) are encoded by an mRNA comprising a frameshift variant expressed by a cancer cell.

47. The method of claim 46, wherein the frameshift variant is created in a transcription insertion or deletion error in a microsatellite.

48. The method of claim 46 or claim 47, wherein the frameshift variant is downstream of a microsatellite.

49. The method of any one of claims 42 to 48, wherein the peptides comprise at least one T cell epitope.

50. The method of any one of claims 42 to 49, wherein the peptides comprise at least one B cell epitope.

51. The method of any one of claims 42 to 50, wherein the peptides are administered as a nucleic acid encoding the peptides.

52. The method of any one of claims 42 to 51, wherein the peptides are administered in a vaccine composition.

53. The method of claim 52, wherein the vaccine composition comprises an adjuvant.

54. The method of claim 53, wherein the adjuvant is selected from the group consisting of ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan.

55. The method of any one of claims 42 to 54, wherein the method further comprises administration of a checkpoint inhibitor.

56. The method of claim 55, wherein the immune checkpoint inhibitor is selected from the group consisting of a PD-1 inhibitor, a PD-L1 inhibitor, and a CTLA-4 inhibitor.

57. The method of claim 55 or claim 56, wherein the immune checkpoint inhibitor is selected from one or more of the group consisting of Pembrolizumab, Nivolumab, and Atezolizumab.

58. The method of any one of claims 42 to 57, wherein peptides are administered via a route selected from the group consisting of subcutaneous, oral, intradermal, intramuscular, intranasal, intravenous, and sublingual.

59. The method of any one of claims 42 to 58, wherein the individual is a mammal.

60. The method of any one of claims 42 to 59, wherein the individual is a human, a dog, a cat, a mouse, a rat, a rabbit, a horse, a cow, or a pig.

61. The method of any one of claims 42 to 60, wherein the immune response is directed to a cancer.

62. The method of claim 61, wherein the cancer is selected from the group consisting of Acute lymphoblastic leukemia, Acute monocytic leukemia, Acute myeloid leukemia, Acute promyelocytic leukemia, Adenocarcinoma, Adult T-cell leukemia, Astrocytoma, Bladder cancer, Bone Cancer, Brain Tumor, Breast Cancer, Burkitt's lymphoma, Carcinoma, Cervical Cancer, Chronic Lymphocytic Leukemia, Chronic myelogenous leukemia, Colon Cancer, Colorectal cancer, Endometrial cancer, Glioblastoma multiforme, Glioma, Hepatocellular carcinoma, Hodgkin's lymphoma, Inflammatory breast cancer, Kidney Cancer, Leukemia, Lung cancer, Lymphoma, Malignant Mesothelioma, Medulloblastoma, Melanoma, Multiple myeloma, Neuroblastoma, Non-Hodgkin Lymphoma, Non-Small Cell Lung Cancer, Ovarian Cancer, Pancreatic Cancer, Pituitary tumor, Prostate cancer, Retinoblastoma, Skin Cancer, Small Cell Lung Cancer, Squamous cell carcinoma, Stomach cancer, T-cell leukemia, T-cell lymphoma, Thyroid cancer, and Wilms' tumor.

63. A cancer vaccine composition comprising one or more peptides selected from the group consisting of SEQ ID NO: 1-6554 and an adjuvant.

64. The composition of claim 63, wherein the adjuvant is selected from the group consisting of ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan.

65. The composition of claim 63 or claim 64, wherein the composition is formulated for intramuscular or subcutaneous administration.

66. The composition of any one of claims 63 to 65, wherein the composition further comprises a checkpoint inhibitor.

67. A cancer vaccine composition comprising a nucleic acid comprising at least one constitutive promoter, and encoding one or more peptides selected from the group consisting of SEQ ID NO: 1-6554.

68. The composition of claim 67, wherein the composition comprises an adjuvant.

69. The composition of claim 67 or claim 68, wherein the adjuvant is selected from the group consisting of ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan.

70. The method of any one of claims 67 to 69, wherein the composition is formulated for intramuscular or subcutaneous administration.

71. The method of any one of claims 67 to 70, wherein the composition comprises a checkpoint inhibitor.
